(19) United States
(12) Patent Application Publication
(10) Pub. No.: US 2024/0368159 A1
(43) Pub. Date: Nov. 7, 2024

(54) HPK1 DEGRADERS, COMPOSITIONS COMPRISING THE HPK1 DEGRADER, AND METHODS OF USING THE SAME

(71) Applicant: BIOFRONT LTD, Grand Cayman (KY)

(72) Inventors: Tianwei MA, Beijing (CN); Lichao FANG, Beijing (CN); Feng SHI, Beijing (CN); Yayi WANG, Beijing (CN); Wei XUE, Beijing (CN); Miao LIU, Beijing (CN); Zheng HUANG, Beijing (CN)

(21) Appl. No.: 18/684,860

(22) PCT Filed: Aug. 22, 2022
(86) PCT No.: PCT/CN2022/113919

§ 371 Date: Feb. 20, 2024

(30) Foreign Application Priority Data
Aug. 24, 2021 (WO) PCT/CN2021/114321

(51) Int. Cl. C07D 471/04; A61K 31/496; A61K 31/506; C07D 519/00

(52) U.S. Cl. CPC C07D 471/04; A61K 31/496; A61K 31/506; C07D 519/00

(57) ABSTRACT

Provided compounds of Formula I and I′, pharmaceutical compositions comprising the compounds, and methods of using the same, in treating, for example, the diseases, disorders, or conditions mediated by the degradation of a protein kinase, such as Hematopoietic progenitor kinase 1 (HPK1).

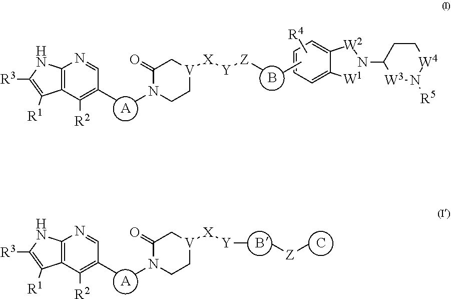

### FIELD OF THE INVENTION

This disclosure relates to novel compounds that are useful for treatment of certain diseases. Specifically, this disclosure relates to novel compounds that bind hematopoietic progenitor kinases (HPKs), such as HPK1, and induce HPK1 degradation, and treat HPK1 dependent disorders.

### BACKGROUND OF THE INVENTION

Hematopoietic progenitor kinase 1 (HPK1), also known as MAP4K1, is a serine/threonine kinase and is predominantly expressed in hematopoietic cells, such as T cells, B cells, and dendritic cells (DC). HPK1 kinase activity can be induced by a variety of receptor stimulations, including, for example, TCR, BCR, EP2/4, and CD95 (Sawasdikosol & Burakoff, 2020). Upon the TCR engagement, HPK1 is phosphorylated at tyrosine 379 by ZAP70, allowing binding with SH2 domain of SLP76. HPK1 subsequently phosphorylates serine 376 of SLP76 and threonine 262 of Gads (Di Bartolo et al., 2007; Lasserre et al., 2011), creating binding sites for 14-3-3 disruption of SLP76 and LAT complex (di Bartolo et al., 2007; Lasserre et al., 2011). This acts as negative feedback signaling to TCR activation. The functions of HPK1 have been validated by various genetic evidence. HPK1−/− T cells have lower activation threshold with increased pro-inflammatory cytokine and hyper-proliferative response (Liu et al., 2019). HPK1−/− T cells also exhibit resistance to PGE2-mediated suppression (Alzabin et al., 2009). HPK1−/− dendritic cells have demonstrated superior antigen presentation ability in vitro, leading to anti-tumor responses in vivo. In addition, the HPK1−/− mice showed better anti-tumor activity than the wild type mice in several tumor models (Liu et al., 2019). These highlight the importance of HPK1's activity in enhancing immune cells' functions and preventing the tumor progression. In addition to autoimmune diseases, it was also reported that MAP4K1 expression is a novel resistance mechanism and independent prognostic marker in AML (Knight et al; 2021; Ling et al, 2021). Thus, HPK1 can be a novel target for cancer and other disorders.

Structurally, HPK1 comprises N-terminal kinase domain, proline-rich domain, and C-terminal citron homology domain. Traditionally, HPK1 activity can be modulated by kinase domain. HPK1 binds many adaptor proteins, including, for example, Grb2, Nck, Crk, and SLP-76, and actin-binding adaptors HIP-55. The proline-rich domain can bind proteins that contain SH3 domains. HPK1 can interact with IKK-α/β to prevent complex formation of ADAP and SLP76. A bivalent heterobifunctional molecule, also known as proteolysis-targeted chimeras (PROTAC), in addition to inhibiting enzymatic activity, also eliminates scaffolding functions of the protein. Thus, a molecule that binds HPK1 and induces its degradation can not only have better efficacy than inhibition of the kinase activity, but also overcome the inhibition induced expression or acquired resistance.

### SUMMARY OF THE INVENTION

One aspect of the present disclosure provides a compound selected from compounds of Formula I, a tautomer thereof, a deuterated derivative of the compound or the tautomer, or a pharmaceutically acceptable salt of the foregoing, which can be employed in the treatment of diseases mediated by the degradation of hematopoietic progenitor kinase 1 (HPK1). For example, disclosed herein is a compound of the following structural Formula I:

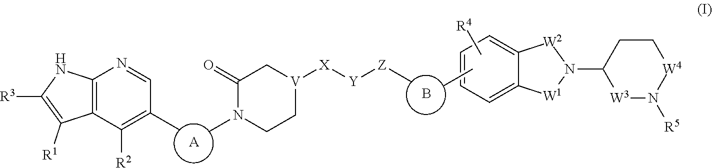

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) each R², R³ and R⁴ is independently chosen from hydrogen, halogen groups, OR^{x}, SR^{x}, NHR^{x}, N(R^{x})₂, CHR^{x}, and C(R^{x})₂;
(iii) R⁵ is chosen from hydrogen, R^{x}, —CH₂OC(O)R^{x}—, and —CH₂OC(O)C(R^{x}R^{y})NH₂;
(iv) each W¹, W², W³, and W⁴ is independently chosen from C(R^{w})₂ and C(O);
(v) V is chosen from N and CR^{x};
(vi) when V is N, X is absent or is chosen from —C(O)—, —C(O)R^{x}—, —C(S)—, —C(S) R^{x}—, —S(O)₂—, and —S(O)₂R^{x}—; or when V is CR^{x}, X is absent or is chosen from —O—, —S—, —NR^{x}—, —C(O)—, —C(S)—, and —C(R^{x}R^{y}),
(vii) Y is absent or is chosen from linear, branched, and cyclic alkylene groups and PEG groups;
(viii) Z is absent or is chosen from —O—, —NR^{z}—, —NR^{y}C(O)—, —C(O)—, —C(S)—, and —C(O)O—;
(ix) each R^{w}, R^{x}, R^{y}, and R^{z} is independently chosen from hydrogen, linear, branched, and cyclic alkyl groups, carbocyclic groups, heterocyclic groups, aryl groups, and heteroaryl groups;
(x) ring A is chosen from aryl groups and heteroaryl groups, and
(xi) ring B is absent or is chosen from aryl groups, heteroaryl groups, cycloalkyl groups, and heterocycloalkyl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups.

One aspect of the present disclosure provides a compound selected from compounds of Formula I′, a tautomer thereof, a deuterated derivative of the compound or the tautomer, or a pharmaceutically acceptable salt of the foregoing, which can be employed in the treatment of diseases mediated by the degradation of hematopoietic progenitor kinase 1 (HPK1). For example, disclosed herein is a compound of the following structural Formula I′:

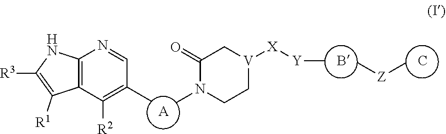

a tautomer thereof, a deuterated derivative of the compound or the tautomer, or a pharmaceutically acceptable salt the foregoing, wherein:
(xii) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R™, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(xiii) each R² and R³ is independently chosen from hydrogen, halogen groups, OR^{x}, SR^{x}, NHR^{x}, N(R^{x})₂, CHR^{x}, and C(R^{x})₂;
(xiv) V is chosen from N and CR^{x};
(xv) when V is N, X is absent or is chosen from —C(O)—, —C(O)R^{x}—, —C(S)—, —C(S) R^{x}—, —S(O)₂—, and —S(O)₂R^{x}—; or when V is CR^{x}, X is absent or is chosen from —O—, —S—, —NR^{x}—, —C(O)—, —C(S)—, and C(R^{x}R^{y}),
(xvi) Y is absent or is chosen from linear, branched, and cyclic alkylene groups and PEG groups;
(xvii) Z is absent or is chosen from —O—, —NR^{z}—, —NR^{y}C(O)—, —C(O)—, C(S), and C(O)O—;
(xviii) each R^{w}, R^{x}, R^{y}, and R^{z} is independently chosen from hydrogen, linear, branched, and cyclic alkyl groups, carbocyclic groups, heterocyclic groups, aryl groups, and heteroaryl groups;
(xix) ring A is chosen from aryl groups and heteroaryl groups,
(xx) ring B′ is absent or is chosen from aryl groups, heteroaryl groups, cycloalkyl groups, and heterocycloalkyl groups;
(xxi) ring C is chosen from

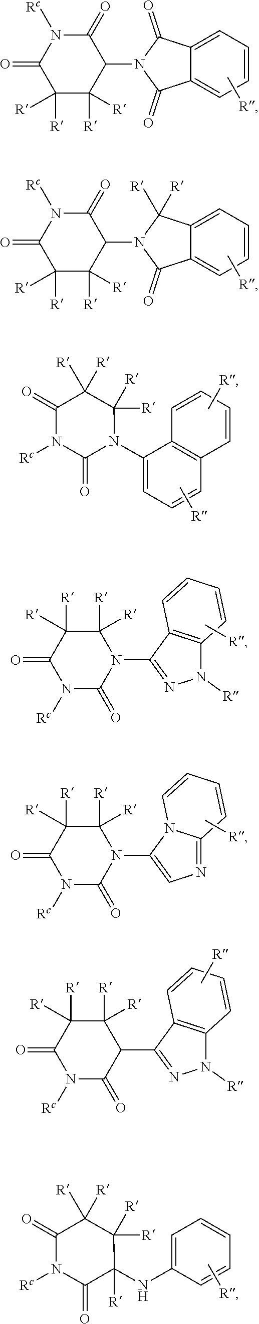

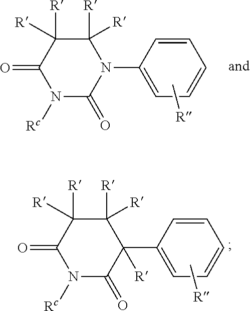

wherein R^{c} is chosen from hydrogen, linear, branched, and cyclic alkyl groups;
each R′ and R″ is independently chosen from hydrogen, halogen groups, OR^{x}, linear, branched, and cyclic alkyl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups.

In one aspect of the present disclosure, the compounds of Formulae I and I′ are selected from Compounds 1 to 106 shown below, a tautomer thereof, a deuterated derivative of the compound or the tautomer, or a pharmaceutically acceptable salt of the foregoing.

In some embodiments, the present disclosure provides pharmaceutical compositions comprising a compound of Formulae I and I′, a tautomer thereof, a deuterated derivative of the compound or the tautomer, or a pharmaceutically acceptable salt of the foregoing, and a pharmaceutically acceptable carrier. In some embodiments, the pharmaceutical compositions may comprise a compound selected from Compounds 1 to 106 shown below, a tautomer thereof, a deuterated derivative of the compound or the tautomer, or a pharmaceutically acceptable salt of the foregoing. These compositions may further comprise an additional active pharmaceutical agent.

Another aspect of the present disclosure provides methods of treating a disease, a disorder, or a condition mediated by the degradation of hematopoietic progenitor kinase 1 (HPK1) in a subject, comprising administering a therapeutically effective amount of a compound of Formulae I and I′, a tautomer thereof, a deuterated derivative of the compound or the tautomer, or a pharmaceutically acceptable salt of the foregoing, or a pharmaceutical composition comprising any of the foregoing. In some embodiments, the methods of treatment comprise administering to a subject, a compound selected from Compounds 1 to 106 shown below, a tautomer thereof, a deuterated derivative of the compound or the tautomer, or a pharmaceutically acceptable salt of the foregoing, or a pharmaceutical composition comprising any of the foregoing.

In some embodiments disclosed herein, the methods of treatment comprise administration of an additional active pharmaceutical agent to the subject in need thereof, either in the same pharmaceutical composition as a compound of Formulae I and I′, a tautomer thereof, a deuterated derivative of the compound or the tautomer, or a pharmaceutically acceptable salt of the foregoing, or in a separate composition. In some embodiments disclosed herein, the methods of treatment comprise administering a compound selected from Compounds 1 to 106 shown below, a tautomer thereof, a deuterated derivative of the compound or the tautomer, or a pharmaceutically acceptable salt of the foregoing with an additional active pharmaceutical agent either in the same composition or in a separate composition.

Also disclosed herein are methods of decreasing HPK1 activities, comprising administering to a subject a therapeutically effective amount of a compound of Formulae I and I′, a tautomer thereof, a deuterated derivative of the compound or the tautomer, or a pharmaceutically acceptable salt of the foregoing, or a pharmaceutical composition comprising any of the foregoing. In some embodiments disclosed herein, the methods of degrading HPK1 comprise administering to a subject, a compound selected from Compounds 1 to 106 shown below, a tautomer thereof, a deuterated derivative of the compound or the tautomer, or a pharmaceutically acceptable salt of the foregoing, or a pharmaceutical composition comprising any of the foregoing. In some embodiments, the methods of decreasing HPK1 activity comprise contacting said HPK1 with a compound of Formulae I and I′, a tautomer thereof, a deuterated derivative of the compound or the tautomer, or a pharmaceutically acceptable salt of the foregoing, or a pharmaceutical composition comprising any of the foregoing. In some embodiments disclosed herein, the methods of degrading HPK1 comprise contacting the HPK1 with a compound selected from Compounds 1 to 106 shown below, a tautomer thereof, a deuterated derivative of the compound or the tautomer, or a pharmaceutically acceptable salt of the foregoing, or a pharmaceutical composition comprising any of the foregoing.

### BRIEF DESCRIPTION OF THE FIGURES

The foregoing summary, as well as the following detailed description of the disclosure, will be better understood when read in conjunction with the appended drawings. For the purpose of illustrating the present disclosure, the appended drawings illustrate some, but not all, alternative embodiments. It should be understood, however, that the disclosure is not limited to the precise arrangements and instrumentalities shown. These drawings, which are incorporated into and constitute part of the specification, assist in explaining the principles of the disclosure.

FIG. 1 illustrates the IL-2 production in primary mouse CD3+ T cells.

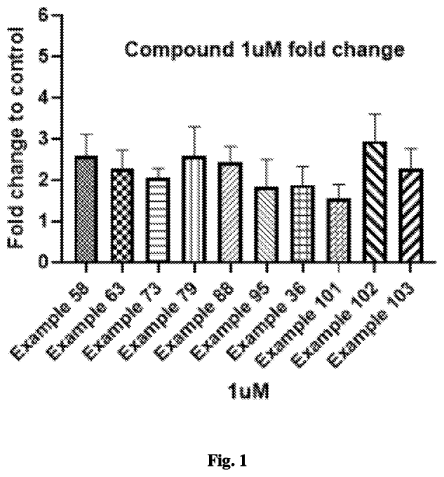

FIG. 2 illustrates the HPK1 degradation by Example 1 of the present disclosure in primary mouse CD3+ T cells.

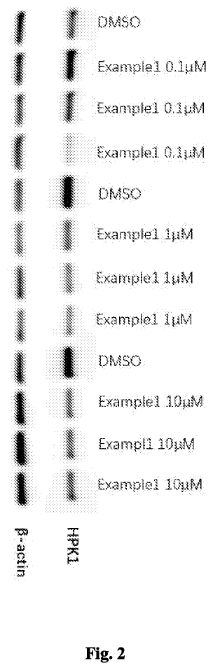

FIG. 3 illustrates the HPK1 degradation by Example 16 of the present disclosure in primary mouse CD3+ T cells.

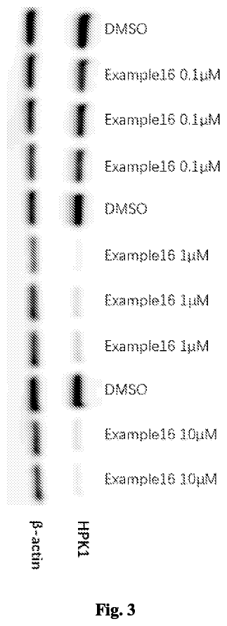

FIG. 4 illustrates the HPK1 degradation by Example 21 of the present disclosure in primary mouse CD3+ T cells.

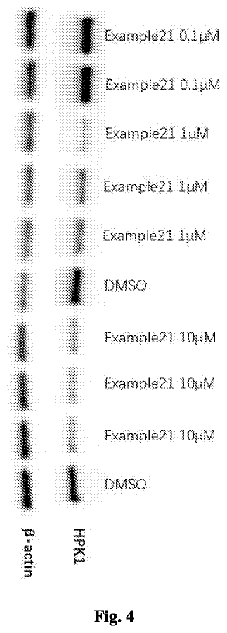

FIG. 5 illustrates the HPK1 degradation by Example 31 of the present disclosure in primary mouse CD3+ T cells.

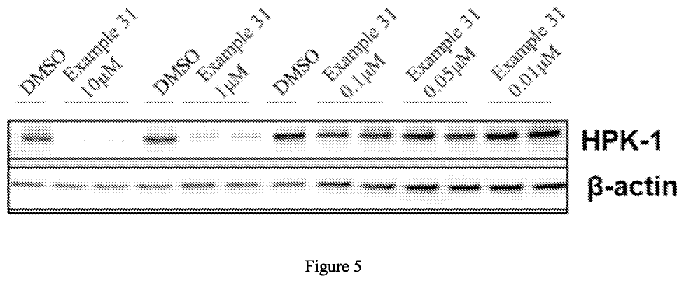

FIG. 6 illustrates the HPK1 degradation by Example 35 of the present disclosure in primary mouse CD3+ T cells.

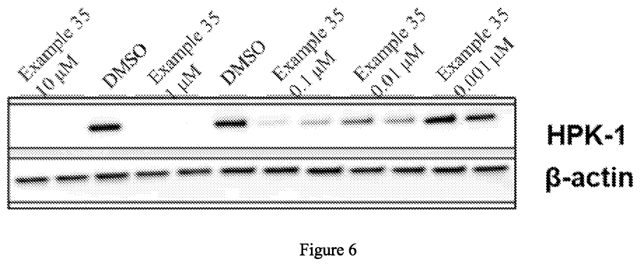

FIG. 7 illustrates the HPK1 degradation by Example 51 of the present disclosure in primary mouse CD3+ T cells.

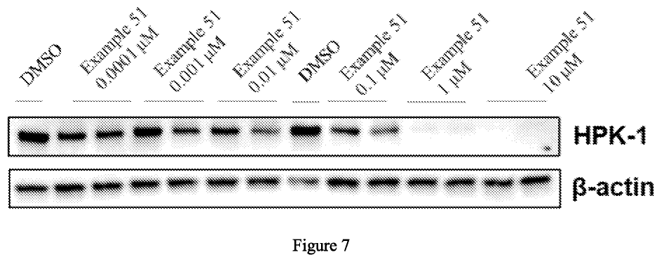

FIG. 8 illustrates the HPK1 degradation by Example 56 of the present disclosure in primary mouse CD3+ T cells.

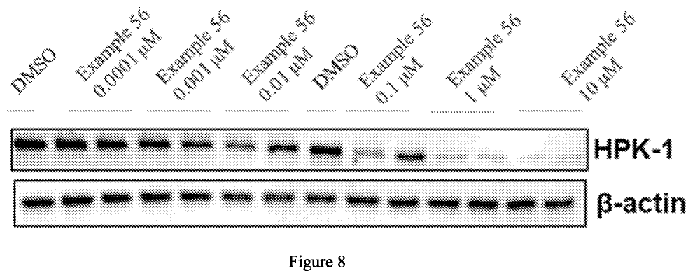

FIG. 9 illustrates the HPK1 degradation by Example 79 of the present disclosure in primary mouse CD3+ T cells.

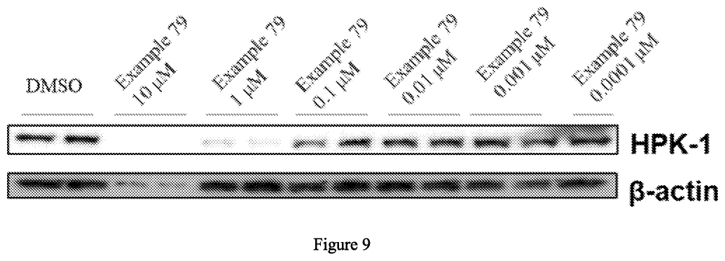

FIG. 10 illustrates the HPK1 degradation by Example 31 of the present disclosure in human PBMC.

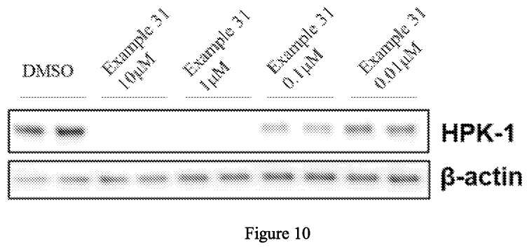

FIG. 11 illustrates the HPK1 degradation by Example 58 of the present disclosure in human PBMC.

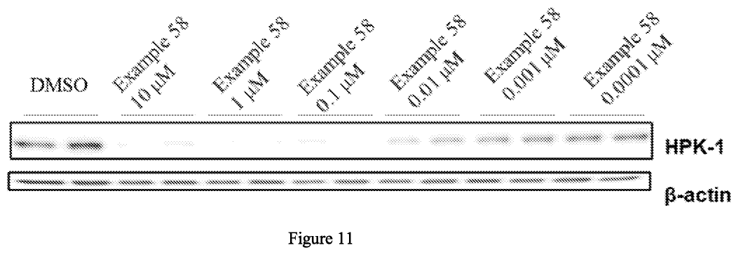

FIG. 12 illustrates the HPK1 degradation by Example 63 of the present disclosure in human PBMC.

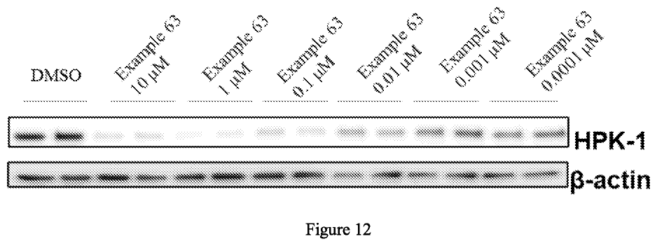

FIG. 13 illustrates the HPK1 degradation by Example 73 of the present disclosure in human PBMC.

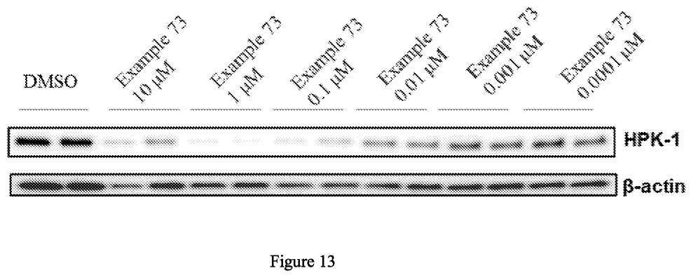

FIG. 14 illustrates the HPK1 degradation by Example 78 of the present disclosure in human PBMC.

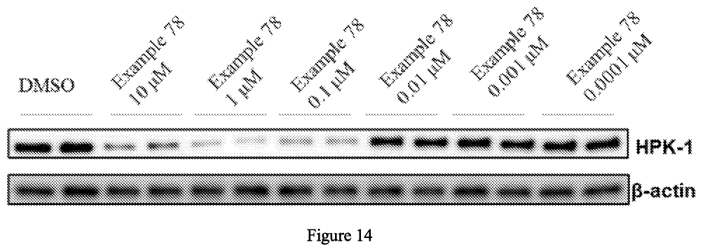

### DETAILED DESCRIPTION

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “protein kinase” is an enzyme that catalyzes the phosphorylation of hydroxyl groups on tyrosine, serine, and threonine residues of proteins. Serine/threonine kinases, specific for phosphorylation of serine and threonine residues, constitute an important family of protein kinases.

The term “HPK1” or “hematopoietic progenitor kinase 1” as used herein, also known as MAP4K1, is a serine/threonine kinase and is predominantly expressed in hematopoietic cells, such as T cells, B cells and dendritic cells (DC). HPK1 is involved in the modulation of various downstream signaling pathways, such as extracellular signal-regulated kinase (ERK), c-Jun N-terminal kinase (JNK), and nuclear factor-κB (NF-κB), which are all associated with the regulation of cellular proliferation and immune cell activation.

Compounds disclosed herein can degrade the protein kinase HPK1. Thus, compounds disclosed herein are generally useful in the treatment of diseases or conditions associated with such a kinase. In one embodiment, the compounds disclosed herein are HPK1 degraders, and are useful for treating diseases, such as cancer, associated with such a kinase.

The term “degrader” as used herein, refers to a molecule agent that binds to hematopoietic progenitor kinase 1 and subsequently lowers the steady state protein levels of the kinase. In some embodiments, a degrader as disclosed herein lowers steady state HPK1 protein levels by at least 10%, at least 20%, at least 30%, at least 40%, at least 50%, at least 60%, at least 65%, at least 70%, at least 75%, at least 80%, at least 85%, at least 90%, at least 95%, at least 96%, at least 97%, at least 98%, or at least 99%. In some embodiments, a degrader as disclosed herein lowers steady state HPK1 protein levels by at least 65%. In some embodiments, a degrader as disclosed herein lowers steady state HPK1 protein levels by at least 85%.

The term “compound,” when referring to a compound of the present disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of the present disclosure will depend upon a number of factors, including, for example, the isotopic purity of reagents used to make the compound and the efficiency of incorporation of isotopes in the various synthesis steps used to prepare the compound. However, as set forth above the relative amount of such isotopologues in to will be less than 49.9% of the compound. In other embodiments, the relative amount of such isotopologues in to will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by the present disclosure are those that result in the formation of stable or chemically feasible compounds.

The term “isotopologue” refers to a species in which the chemical structure differs from only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C are within the scope of the present disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the present disclosure. Unless otherwise stated, all tautomeric forms of the compounds of the present disclosure are within the scope of the present disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer” as used herein refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives disclosed herein. Thus, unless otherwise stated, when a reference is made to a “deuterated derivative” of a compound of the present disclosure, at least one hydrogen is replaced with deuterium at a level that is well above its natural isotopic abundance, which is typically about 0.015%. In some embodiments, the deuterated derivatives disclosed herein have an isotopic enrichment factor for each deuterium atom, of at least 3500 (52.5% deuterium incorporation at each designated deuterium), at least 4500 (67.5% deuterium incorporation at each designated deuterium), at least 5000 (75% deuterium incorporation at each designated deuterium), at least 5500 (82.5% deuterium incorporation at each designated deuterium), at least 6000 (90% deuterium incorporation at each designated deuterium), at least 6333.3 (95% deuterium incorporation at each designated deuterium), at least 6466.7 (97% deuterium incorporation at each designated deuterium), or at least 6600 (99% deuterium incorporation at each designated deuterium).

The term “isotopic enrichment factor” as used herein means the ratio between the isotopic abundance and the natural abundance of a specified isotope.

The term “alkyl” as used herein, means a linear or branched, substituted or unsubstituted hydrocarbon chain that is completely saturated. Unless otherwise specified, an alkyl group contains 1 to 30 alkyl carbon atoms. In some embodiments, an alkyl group contains 1 to 20 alkyl carbon atoms. In some embodiments, an alkyl group contains 1 to 10 aliphatic carbon atoms. In some embodiments, an alkyl group contains 1 to 8 aliphatic carbon atoms. In some embodiments, an alkyl group contains 1 to 6 alkyl carbon atoms. In some embodiments, an alkyl group contains 1 to 4 alkyl carbon atoms. In other embodiments, an alkyl group contains 1 to 3 alkyl carbon atoms. And in yet other embodiments, an alkyl group contains 1 to 2 alkyl carbon atoms. In some embodiments, alkyl groups are substituted. In some embodiments, alkyl groups are unsubstituted. In some embodiments, alkyl groups are linear or straight-chain or unbranched. In some embodiments, alkyl groups are branched.

The term “cycloalkyl” refers to a monocyclic C₃₋₈ hydrocarbon or a spirocyclic, fused, or bridged bicyclic or tricyclic C₈₋₁₄ hydrocarbon that is completely saturated, wherein any individual ring in said bicyclic ring system has 3 to 7 members. In some embodiments, cycloalkyl groups are substituted. In some embodiments, cycloalkyl groups are unsubstituted. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentyl, and cyclohexyl.

The term “carbocyclyl” encompasses the term “cycloalkyl” and refers to a monocyclic C₃₋₈ hydrocarbon or a spirocyclic, fused, or bridged bicyclic or tricyclic C₈₋₁₄ hydrocarbon that is completely saturated, or is partially saturated as it contains one or more units of unsaturation but is not aromatic, wherein any individual ring in said bicyclic ring system has 3 to 7 members. Bicyclic carbocyclyls include combinations of a monocyclic carbocyclic ring fused to, for example, a phenyl. In some embodiments, carbocyclyl groups are substituted. In some embodiments, carbocyclyl groups are unsubstituted. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl. Non-limiting examples of monocyclic carbocyclyls include cyclopropyl, cyclobutyl, cyclopentanyl, cyclohexyl, cyclopentenyl, cyclohexenyl, etc.

The term “alkylene” as used herein, refers to a divalent alkyl radical. Representative examples of C₁₋₁₀ alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene.

The term “alkenyl” as used herein, means a linear or branched, substituted or unsubstituted hydrocarbon chain that contains one or more double bonds. In some embodiments, alkenyl groups are substituted. In some embodiments, alkenyl groups are unsubstituted. In some embodiments, alkenyl groups are linear, straight-chain, or unbranched. In some embodiments, alkenyl groups are branched.

The term “alkynyl” as used herein, refers to an unsaturated straight or branched hydrocarbon having at least one carbon-carbon triple bond, such as a straight or branched group of 2 to 8 carbon atoms, referred to herein as C₂₋₈alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “heterocyclyl” as used herein means non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include, for example, the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl. In some embodiments, the “heterocyclyl” group contains 3 to 14 ring members in which one or more ring members is a heteroatom independently chosen, for example, from oxygen, sulfur, nitrogen, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, and phosphorus. In some embodiments, the heterocycle has one heteroatom that is a nitrogen atom. In some embodiments, the heterocycle has one heteroatom that is an oxygen atom. In some embodiments, the heterocycle has two heteroatoms that are each independently selected from nitrogen and oxygen. In some embodiments, the heterocycle has three heteroatoms that are each independently selected from nitrogen and oxygen. In some embodiments, heterocycles are substituted. In some embodiments, heterocycles are unsubstituted. In some embodiments, the heterocyclyl is a 3- to 12-membered heterocyclyl. In some embodiments, the heterocyclyl is a 4- to 10-membered heterocyclyl. In some embodiments, the heterocyclyl is a 3- to 8-membered heterocyclyl. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. In some embodiments, the heterocyclyl is a 6-membered heterocyclyl. Non-limiting examples of monocyclic heterocyclyls include piperidinyl, piperazinyl, morpholinyl, tetrahydropyranyl, azetidinyl, oxetanyl, tetrahydrothiophenyl, dihyropyranyl, tetrahydropyridinyl, etc.

The term “heteroatom” means one or more of oxygen, sulfur, and nitrogen, including, any oxidized form of nitrogen or sulfur, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains double or triple bonds.

The term “alkoxy” as used herein, refers to an alkyl group, as defined above, wherein one carbon of the alkyl group is replaced by an oxygen (“alkoxy”) atom, provided that the oxygen atom is linked between two carbon atoms.

The term “halogen” includes F, Cl, Br, and I, i.e., fluoro, chloro, bromo, and iodo, respectively.

As used herein, a “cyano” or “nitrile” group refer to —C≡N.

As used herein, an “aromatic ring” refers to a carbocyclic or heterocyclic ring that contains conjugated, planar ring systems with delocalized pi electron orbitals comprised of [4n+2] p orbital electrons, wherein n is an integer of 0 to 6. A “non-aromatic” ring refers to a carbocyclic or heterocyclic that does not meet the requirements set forth above for an aromatic ring, and can be either completely or partially saturated. Nonlimiting examples of aromatic rings include aryl and heteroaryl rings that are further defined as follows.

The term “aryl” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Nonlimiting examples of aryl groups include phenyl (C₆) and naphthyl (C₁₀) rings. In some embodiments, aryl groups are substituted. In some embodiments, aryl groups are unsubstituted.

The term “heteroaryl” refers to monocyclic or spirocyclic, fused, or bridged bicyclic or tricyclic ring systems having a total of five to fourteen ring members, wherein at least one ring in the system is aromatic, at least one ring in the system contains one or more heteroatoms, and wherein each ring in a bicyclic or tricyclic ring system contains 3 to 7 ring members. Bicyclic heteroaryl groups include, for example, the following combinations of monocyclic rings: a monocyclic heteroaryl fused to another monocyclic heteroaryl; and a monocyclic heteroaryl fused to a phenyl. In some embodiments, heteroaryl groups are substituted. In some embodiments, heteroaryl groups have one or more heteroatoms chosen, for example, from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, heteroaryl groups are unsubstituted. In some embodiments, the heteroaryl is a 3- to 12-membered heteroaryl. In some embodiments, the heteroaryl is a 3- to 10-membered heteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. Non-limiting examples of monocyclic heteroaryl groups are pyridinyl, pyrimidinyl, thiophenyl, thiazolyl, isoxazolyl, etc.

A “spirocyclic ring system” refers to a ring system having two or more cyclic rings, where every two rings share only one common atom.

Non-limiting examples of suitable solvents that may be used in the present disclosure include water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptane, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in the present disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

Disclosed herein are pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of the present disclosure. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. The present disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The term “subject” refers to an animal, including but not limited to, a human.

The term “therapeutically effective amount” refers to that amount of a compound that produces the desired effect for which it is administered (e.g., improvement in symptoms of diseases, disorders, and conditions mediated by the degradation of HPK1, lessening the severity of diseases, disorders, and conditions mediated by the degradation of HPK1 or a symptom thereof, and/or reducing progression of diseases, disorders, and conditions mediated by the degradation of HPK1 or a symptom thereof). The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, lower risk of diseases, disorders, and conditions mediated by the degradation of HPK1, and disease-related complications. Improvements in or lessening the severity of any of these symptoms can be readily assessed according to methods and techniques known in the art or subsequently developed.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

### II. Compounds and Compositions

In a first embodiment, a compound of the present disclosure is a compound of the following structural formula I:

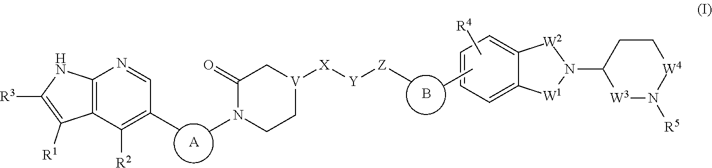

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) each R², R³ and R⁴ is independently chosen from hydrogen, halogen groups, OR^{x}, SR^{x}, NHR^{x}, N(RN)₂, CHR^{x}, and C(R^{x})₂;
(iii) R⁵ is chosen from hydrogen, R^{x}, —CH₂OC(O)R^{x}—, and —CH₂OC(O)C(R^{x}R^{y})NH₂;
(iv) each W¹, W², W³, and W⁴ is independently chosen from C(R^{w})₂ and C(O);
(v) V is chosen from N and CR^{x};
(vi) when V is N, X is absent or is chosen from —C(O)—, —C(O)R^{x}—, —C(S)—, —C(S) R^{x}, —S(O)₂—, and —S(O)₂R^{x}—; or when V is CR^{x}, X is absent or is chosen from —O—, —S—, —NR^{x}—, —C(O)—, —C(S)—, and —C(R^{x}R^{y})—,
(vii) Y is absent or is chosen from linear, branched, and cyclic alkylene groups and PEG groups;
(viii) Z is absent or is chosen from —O—, —NR^{z}—, —NR^{y}C(O)—, —C(O)—, C(S)—, and —C(O)O—;
(ix) each R^{w}, R^{x}, R^{y}, and R^{z} is independently chosen from hydrogen, linear, branched, and cyclic alkyl groups, carbocyclic groups, heterocyclic groups, aryl groups, and heteroaryl groups;
(x) ring A is chosen from aryl groups and heteroaryl groups, and
(xi) ring B is absent or is chosen from aryl groups, heteroaryl groups, cycloalkyl groups, and heterocycloalkyl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups.

In a second embodiment, a compound of the present disclosure is a compound of the following structural formula I′:

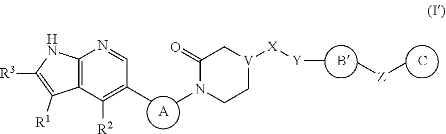

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{y}R^{y}, S(O)R^{y}, and SO₂R^{y};
(ii) each R² and R³ is independently chosen from hydrogen, halogen groups, OR^{x}, SR^{x}, NHR^{x}, N(R^{x})₂, CHR^{x}, and C(R^{x})₂;
(iii) V is chosen from N and CR^{x};
(iv) when V is N, X is absent or is chosen from —C(O)—, —C(O)R^{x}—, —C(S)—, —C(S) R^{x}—, —S(O)₂—, and —S(O)₂R^{x}—; or when V is CR^{x}, X is absent or is chosen from —O—, —S—, —NR^{x}, —C(O)—, —C(S)—, and —C(R^{x}R^{y})—,
(v) Y is absent or is chosen from linear, branched, and cyclic alkylene groups and PEG groups;
(vi) Z is absent or is chosen from —O—, —NR^{z}—, —NR^{y}C(O)—, —C(O)—, —C(S)—, and —C(O)O—;
(vii) each R^{w}, R^{x}, R^{y}, and R^{z} is independently chosen from hydrogen, linear, branched, and cyclic alkyl groups, carbocyclic groups, heterocyclic groups, aryl groups, and heteroaryl groups;
(viii) ring A is chosen from aryl groups and heteroaryl groups,
(ix) ring B′ is absent or is chosen from aryl groups, heteroaryl groups, cycloalkyl groups, and heterocycloalkyl groups;
(x) ring C is chosen from

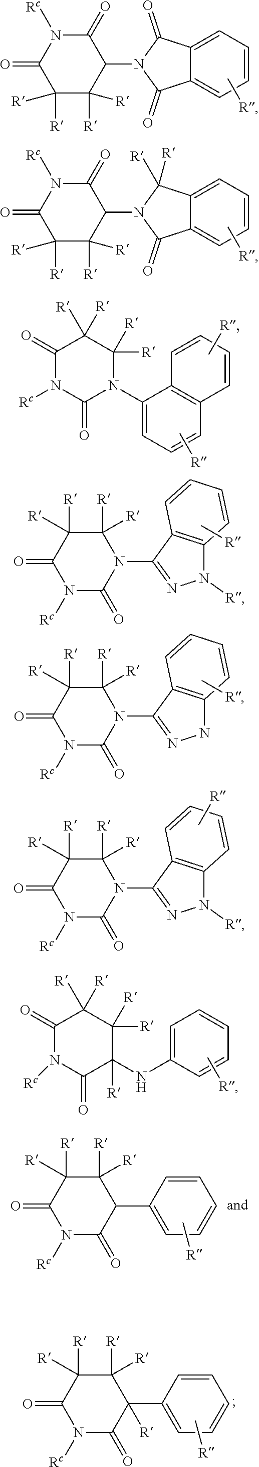

wherein R_{c} is chosen from hydrogen, linear, branched, and cyclic alkyl groups;
each R′ and R″ is chosen from hydrogen, halogen groups, OR^{x}, linear, branched, and cyclic alkyl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups.

In a third embodiment, in a compound, tautomer, deuterated derivative, or pharmaceutically acceptable salt of the present disclosure, R¹ is chosen from linear, branched, and cyclic alkyl groups; R² is a halogen group; and R³ is chosen from hydrogen, linear, branched, and cyclic alkyl groups; and all other variables not specifically defined herein are as defined in the first and second embodiment.

In a fourth embodiment, in a compound, tautomer, deuterated derivative, or pharmaceutically acceptable salt of the present disclosure, R¹ is chosen from C₁-C₆ linear, branched, and cyclic alkyl groups; and all other variables not specifically defined herein are as defined in the all of the preceding embodiments.

In a fifth embodiment, in a compound, tautomer, deuterated derivative, or pharmaceutically acceptable salt of the present disclosure, R¹ is chosen from methyl, ethyl, cyclopropyl, and cyclobutyl; and all other variables not specifically defined herein are as defined in the fourth embodiment.

In a sixth embodiment, in a compound, tautomer, deuterated derivative, or pharmaceutically acceptable salt of the present disclosure, R² is a halogen group; and all other variables not specifically defined herein are as defined in the proceeding embodiments.

In a seventh embodiment, in a compound, tautomer, deuterated derivative, or pharmaceutically acceptable salt of the present disclosure, R² is chloro; and all other variables not specifically defined herein are as defined in the sixth embodiment.

In an eighth embodiment, in a compound, tautomer, deuterated derivative, or pharmaceutically acceptable salt of the present disclosure, R² is hydrogen; and all other variables not specifically defined herein are as defined in any one of the first to the fifth embodiment.

In a ninth embodiment, in a compound, tautomer, deuterated derivative, or pharmaceutically acceptable salt of the present disclosure, R³ is a halogen group; and all other variables not specifically defined herein are as defined in the proceeding embodiments.

In a tenth embodiment, in a compound, tautomer, deuterated derivative, or pharmaceutically acceptable salt of the present disclosure, R³ is chloro; and all other variables not specifically defined herein are as defined in the ninth embodiment.

In a eleventh embodiment, in a compound, tautomer, deuterated derivative, or pharmaceutically acceptable salt of the present disclosure, R³ is hydrogen; and all other variables not specifically defined herein are as defined in any one of the first to the eighth embodiments.

In a twelfth embodiment, in a compound, tautomer, deuterated derivative, or pharmaceutically acceptable salt of the present disclosure, R⁴ is a halogen group; and all other variables not specifically defined herein are as defined in the proceeding embodiments.

In a thirteenth embodiment, in a compound, tautomer, deuterated derivative, or pharmaceutically acceptable salt of the present disclosure, R⁴ is fluoro; and all other variables not specifically defined herein are as defined in the proceeding embodiments.

In a fourteenth embodiment, in a compound, tautomer, deuterated derivative, or pharmaceutically acceptable salt of the present disclosure, R⁵ is hydrogen; and all other variables not specifically defined herein are as defined in any one of the first to the thirteenth embodiments.

In a fifteenth embodiment, in a compound, tautomer, deuterated derivative, or pharmaceutically acceptable salt of the present disclosure, R⁵ is chosen from C₁-C₆ linear, branched, and cyclic alkyl groups; and all other variables not specifically defined herein are as defined in any one of the first to the thirteenth embodiments.

In a sixteenth embodiment, in a compound, tautomer, deuterated derivative, or pharmaceutically acceptable salt of the present disclosure, R⁵ is chosen from methyl, ethyl, n-propyl, i-propyl, n-butyl, i-butyl, and t-butyl; and all other variables not specifically defined herein are as defined in the fifteenth embodiment.

In a seventeenth embodiment, in a compound, tautomer, deuterated derivative, or pharmaceutically acceptable salt of the present disclosure, R⁵ is —CH₂OC(O)R^{x}—; and all other variables not specifically defined herein are as defined in any one of the first to the thirteenth embodiments.

In a eighteenth embodiment, in a compound, tautomer, deuterated derivative, or pharmaceutically acceptable salt of the present disclosure, R^{x} is chosen from methyl, ethyl, n-propyl, i-propyl, n-butyl, i-butyl, and t-butyl; and all other variables not specifically defined herein are as defined in the seventeenth embodiment.

In an nineteenth embodiment, in a compound, tautomer, deuterated derivative, or pharmaceutically acceptable salt of the present disclosure, R⁵ is —CH₂OC(O)C(R^{x}R^{y})NH₂; and all other variables not specifically defined herein are as defined in any one of the first to the thirteenth embodiments.

In a twentieth embodiment, in a compound, tautomer, deuterated derivative, or pharmaceutically acceptable salt of the present disclosure, R^{x} is hydrogen; and all other variables not specifically defined herein are as defined in the nineteenth embodiment.

In a twenty-first embodiment, in a compound, tautomer, deuterated derivative, or pharmaceutically acceptable salt of the present disclosure, R^{x} is chosen from hydrogen, methyl, iso-propyl, and benzyl; and all other variables not specifically defined herein are as defined in the nineteenth and twentieth embodiments.

In a twenty-second embodiment, X is absent; and all other variables not specifically defined herein are as defined in the proceeding embodiments.

In a twenty-third embodiment, in a compound, tautomer, deuterated derivative, or pharmaceutically acceptable salt of the present disclosure, X is —C(O)—; and all other variables not specifically defined herein are as defined in any one of the first to the twenty-first embodiments.

In a twenty-fourth embodiment, in a compound, tautomer, deuterated derivative, or pharmaceutically acceptable salt of the present disclosure, Y is chosen from linear, branched, and cyclic alkylene groups; and all other variables not specifically defined herein are as defined in the proceeding embodiments.

In a twenty-fifth embodiment, in a compound, tautomer, deuterated derivative, or pharmaceutically acceptable salt of the present disclosure, Y is chosen from C₁-C₁₀ linear alkylene groups; and all other variables not specifically defined herein are as defined in the twenty-third embodiment.

In a twenty-sixth embodiment, in a compound, tautomer, deuterated derivative, or pharmaceutically acceptable salt of the present disclosure, Y is chosen from PEG groups; and all other variables not specifically defined herein are as defined in any one of the first to the fifth embodiments.

In a twenty-seventh embodiment, in a compound, tautomer, deuterated derivative, or pharmaceutically acceptable salt of the present disclosure, Y is chosen from

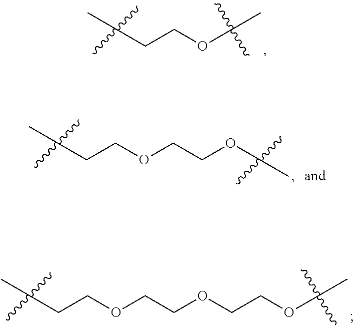 [SMILES: *CCO* ; *CCOCCO* ; *CCOCCOCCO*]

and all other variables not specifically defined herein are as defined in the twenty-fifth embodiment.

In a twenty-eighth embodiment, in a compound, tautomer, deuterated derivative, or pharmaceutically acceptable salt of the present disclosure, Z is absent; and all other variables not specifically defined herein are as defined in the proceeding embodiments.

In a twenty-ninth embodiment, in a compound, tautomer, deuterated derivative, or pharmaceutically acceptable salt of the present disclosure, Z is —C(O)—; and all other variables not specifically defined herein are as defined in any one of the first to the twenty-sixth embodiments.

In a thirtieth embodiment, in a compound, tautomer, deuterated derivative, or pharmaceutically acceptable salt of the present disclosure, Z is O; and all other variables not specifically defined herein are as defined in any one of the first to the twenty-seventh embodiments.

In a thirty-first embodiment, in a compound, tautomer, deuterated derivative, or pharmaceutically acceptable salt of the present disclosure, Z is NR^{z}; and all other variables not specifically defined herein are as defined in any one of the first to the twenty-seventh embodiments.

In a thirty-second embodiment, in a compound, tautomer, deuterated derivative, or pharmaceutically acceptable salt of the present disclosure, R^{z} is chosen from hydrogen, linear, branched, and cyclic alkyl groups; and all other variables not specifically defined herein are as defined in the thirty-first embodiment.

In a thirty-third embodiment, in a compound, tautomer, deuterated derivative, or pharmaceutically acceptable salt of the present disclosure, R^{z} is hydrogen; and all other variables not specifically defined herein are as defined in the thirty-first embodiment.

In a thirty-fourth embodiment, in a compound, tautomer, deuterated derivative, or pharmaceutically acceptable salt of the present disclosure, R^{z} is methyl; and all other variables not specifically defined herein are as defined in the thirtieth embodiment.

In a thirty-fifth embodiment, in a compound, tautomer, deuterated derivative, or pharmaceutically acceptable salt of the present disclosure, ring B is absent; and all other variables not specifically defined herein are as defined in the proceeding embodiments.

In a thirty-sixth embodiment, in a compound, tautomer, deuterated derivative, or pharmaceutically acceptable salt of the present disclosure, ring B is chosen from optionally substituted heterocycloalkyls; and all other variables not specifically defined herein are as defined in the thirty-fifth embodiment.

In a thirty-seventh embodiment, ring B is chosen from

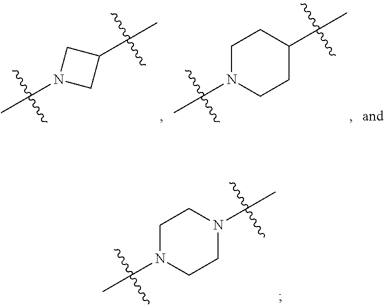 [SMILES: *C1CN(*)C1 ; *C1CCN(*)CC1 ; *N1CCN(*)CC1]

and all other variables not specifically defined herein are as defined in the thirty-sixth embodiment.

In a thirty-eighth embodiment, in a compound, tautomer, deuterated derivative, or pharmaceutically acceptable salt of the present disclosure, W¹ is C(R^{w})₂; and all other variables not specifically defined herein are as defined in the proceeding embodiments, except embodiment 2.

In a thirty-ninth embodiment, in a compound, tautomer, deuterated derivative, or pharmaceutically acceptable salt of the present disclosure, R^{w} is hydrogen; and all other variables not specifically defined herein are as defined in the thirty-eighth embodiment.

In a forty embodiment, in a compound, tautomer, deuterated derivative, or pharmaceutically acceptable salt of the present disclosure, W¹ is C(O); and all other variables not specifically defined herein are as defined in any one of the first to the thirty-sixth embodiments.

In a forty-first embodiment, in a compound, tautomer, deuterated derivative, or pharmaceutically acceptable salt of the present disclosure, W² is C(R^{w})₂; and all other variables not specifically defined herein are as defined in the proceeding embodiments, except embodiment 2.

In a forty-second embodiment, in a compound, tautomer, deuterated derivative, or pharmaceutically acceptable salt of the present disclosure, R^{w} is hydrogen; and all other variables not specifically defined herein are as defined in the forty-first embodiment.

In a forty-third embodiment, in a compound, tautomer, deuterated derivative, or pharmaceutically acceptable salt of the present disclosure, W² is C(O); and all other variables not specifically defined herein are as defined in any one of first to fortieth embodiment.

In a forty-forth embodiment, in a compound, tautomer, deuterated derivative, or pharmaceutically acceptable salt of the present disclosure, W³ is C(R^{w})₂; and all other variables not specifically defined herein are as defined in the proceeding embodiments, except embodiment 2.

In a forty-fifth embodiment, in a compound, tautomer, deuterated derivative, or pharmaceutically acceptable salt of the present disclosure, R^{w} is hydrogen; and all other variables not specifically defined herein are as defined in the forty-forth embodiment.

In a forty-sixth embodiment, in a compound, tautomer, deuterated derivative, or pharmaceutically acceptable salt of the present disclosure, W³ is C(O); and all other variables not specifically defined herein are as defined in the proceeding embodiments, except embodiment 2.

In a forty-seventh embodiment, in a compound, tautomer, deuterated derivative, or pharmaceutically acceptable salt of the present disclosure, W⁴ is C(R^{w})₂; and all other variables not specifically defined herein are as defined in the proceeding embodiments, except embodiment 2.

In a forty-eighth embodiment, in a compound, tautomer, deuterated derivative, or pharmaceutically acceptable salt of the present disclosure, R^{w} is hydrogen; and all other variables not specifically defined herein are as defined in the forty-seventh embodiment.

In a forty-ninth embodiment, in a compound, tautomer, deuterated derivative, or pharmaceutically acceptable salt of the present disclosure, W⁴ is C(O); and all other variables not specifically defined herein are as defined in any one of first to forty-sixth embodiment.

In a fiftieth embodiment, in a compound, tautomer, deuterated derivative, or pharmaceutically acceptable salt of the present disclosure, ring A is a 5 or 6-membered heteroaryl groups; and all other variables not specifically defined herein are as defined in any one of first to forty-sixth embodiment.

In a fifty-first embodiment, in a compound, tautomer, deuterated derivative, or pharmaceutically acceptable salt of the present disclosure, ring A is

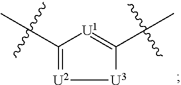

each U¹ and U² is independently chosen from CR^{u} or N; U³ is chosen from O, S, and NR^{u}; and R^{u} is independently chosen from hydrogen, linear, branched, and cyclic alkyl groups, carbocyclic groups, heterocyclic groups, aryl groups, and heteroaryl groups; and all other variables not specifically defined herein are as defined in the fiftieth embodiment.

In a fifty-second embodiment, in a compound, tautomer, deuterated derivative, or pharmaceutically acceptable salt of the present disclosure, U¹ is CR^{u}, U² is CR^{u}, and U³ is O; and all other variables not specifically defined herein are as defined in the fifty-first embodiment.

In a fifty-third embodiment, in a compound, tautomer, deuterated derivative, or pharmaceutically acceptable salt of the present disclosure, U¹ is CR^{u}, U² is CR^{u}, and U³ is S; and all other variables not specifically defined herein are as defined in the fifty-first embodiment.

In a fifty-fourth embodiment, in a compound, tautomer, deuterated derivative, or pharmaceutically acceptable salt of the present disclosure, U¹ is N, U² is CR^{u}, and U³ is O; and all other variables not specifically defined herein are as defined in the fifty-first embodiment.

In a fifty-fifth embodiment, in a compound, tautomer, deuterated derivative, or pharmaceutically acceptable salt of the present disclosure, U¹ is N, U² is CR^{u}, and U³ is S; and all other variables not specifically defined herein are as defined in the fifty-first embodiment.

In a fifty-sixth embodiment, in a compound, tautomer, deuterated derivative, or pharmaceutically acceptable salt of the present disclosure, U¹ is CR^{u}, U² is N, and U³ is O; and all other variables not specifically defined herein are as defined in the fifty-first embodiment.

In a fifty-seventh embodiment, in a compound, tautomer, deuterated derivative, or pharmaceutically acceptable salt of the present disclosure, U¹ is CR^{u}, U² is N, and U³ is S; and all other variables not specifically defined herein are as defined in the fifty-first embodiment.

In a fifty-eighth embodiment, in a compound, tautomer, deuterated derivative, or pharmaceutically acceptable salt of the present disclosure, ring A is

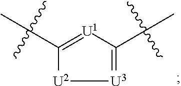

each U¹ and U³ is independently chosen from CR^{u} or N; and U² is chosen from O, S, and NR^{u}; and R^{u} is independently chosen from hydrogen, linear, branched, and cyclic alkyl groups, carbocyclic groups, heterocyclic groups, aryl groups, and heteroaryl groups; and all other variables not specifically defined herein are as defined in the fortieth embodiment.

In a fifty-ninth embodiment, in a compound, tautomer, deuterated derivative, or pharmaceutically acceptable salt of the present disclosure, U¹ is CR^{u}, U² is O, and U³ is CR^{u}, and all other variables not specifically defined herein are as defined in the fifty-eighth embodiment.

In a sixtieth embodiment, in a compound, tautomer, deuterated derivative, or pharmaceutically acceptable salt of the present disclosure, U¹ is CR^{u}, U² is S, and U³ is CR^{u}; and all other variables not specifically defined herein are as defined in the fifty-seventh embodiment.

In a sixty-first embodiment, in a compound, tautomer, deuterated derivative, or pharmaceutically acceptable salt of the present disclosure, U¹ is N, U² is O, and U³ is CR^{u}; and all other variables not specifically defined herein are as defined in the fifty-eighth embodiment.

In a sixty-second embodiment, in a compound, tautomer, deuterated derivative, or pharmaceutically acceptable salt of the present disclosure, U¹ is N, U² is S, and U³ is CR^{u}; and all other variables not specifically defined herein are as defined in the fifty-eighth embodiment.

In a sixty-third embodiment, in a compound, tautomer, deuterated derivative, or pharmaceutically acceptable salt of the present disclosure, U¹ is CR^{z}, U² is O, and U³ is N; and all other variables not specifically defined herein are as defined in the fifty-eighth embodiment.

In a sixty-fourth embodiment, in a compound, tautomer, deuterated derivative, or pharmaceutically acceptable salt of the present disclosure, U¹ is CR^{z}, U² is S, and U³ is N; and all other variables not specifically defined herein are as defined in the fifty-eighth embodiment.

In a sixty-fifth embodiment, in a compound, tautomer, deuterated derivative, or pharmaceutically acceptable salt of the present disclosure, ring A is

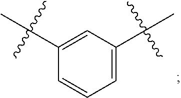 [SMILES: *c1cccc(*)c1]

and all other variables not specifically defined herein are as defined in the fifty-first embodiment.

In a sixty-sixth embodiment, in a compound, tautomer, deuterated derivative, or pharmaceutically acceptable salt of the present disclosure, ring A is

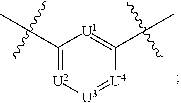

each U¹, U², U³, and U⁴ is independently chosen from CR^{z} or N; and R^{u} is independently chosen from hydrogen, linear, branched, and cyclic alkyl groups, carbocyclic groups, heterocyclic groups, aryl groups, and heteroaryl groups; and all other variables not specifically defined herein are as defined in the fiftieth embodiment.

In a sixty-seventh embodiment, in a compound, tautomer, deuterated derivative, or pharmaceutically acceptable salt of the present disclosure, U¹ is N, U² is CR^{u}, U³ is CR^{u}, and U⁴ is CR^{u}; and all other variables not specifically defined herein are as defined in the sixty-sixth embodiment.

In a sixty-eighth embodiment, in a compound, tautomer, deuterated derivative, or pharmaceutically acceptable salt of the present disclosure, U¹ is CR^{u}, U² is N, U³ is CR^{u}, and U⁴ is CR^{u}; and all other variables not specifically defined herein are as defined in the sixty-sixth embodiment.

In a sixty-ninth embodiment, in a compound, tautomer, deuterated derivative, or pharmaceutically acceptable salt of the present disclosure, U¹ is CR^{u}, U² is CR^{u}, U³ is N, and U⁴ is CR^{u}; and all other variables not specifically defined herein are as defined in the sixty-sixth embodiment.

In a seventieth embodiment, in a compound, tautomer, deuterated derivative, or pharmaceutically acceptable salt of the present disclosure, U¹ is CR^{u}, U² is CR^{u}, U³ is CR^{u}, and U⁴ is N; and all other variables not specifically defined herein are as defined in the sixty-sixth embodiment.

In a seventy-first embodiment, in a compound, tautomer, deuterated derivative, or pharmaceutically acceptable salt of the present disclosure, U¹ is N, U² is N, U³ is CR^{u}, and U⁴ is CR^{u}, and all other variables not specifically defined herein are as defined in the sixty-sixth embodiment.

In a seventy-second embodiment, in a compound, tautomer, deuterated derivative, or pharmaceutically acceptable salt of the present disclosure, U¹ is N, U² is CR^{u}, U³ is N, and U⁴ is CR^{u}; and all other variables not specifically defined herein are as defined in the sixty-sixth embodiment.

In a seventy-third embodiment, in a compound, tautomer, deuterated derivative, or pharmaceutically acceptable salt of the present disclosure, U¹ is CR^{u}, U² is CR^{u}, U³ is CR^{u}, and U is N; and all other variables not specifically defined herein are as defined in the sixty-sixth embodiment.

In a seventy-fourth embodiment, in a compound, tautomer, deuterated derivative, or pharmaceutically acceptable salt of the present disclosure, U¹ is N, U² is CR^{u}, U³ is CR^{u}, and U⁴ is N; and all other variables not specifically defined herein are as defined in the sixty-sixth embodiment.

In certain embodiments, the at least one compound of the present disclosure is selected from Compounds 1 to 106 shown in Table 1 below, a tautomer thereof, a deuterated derivative of the compound or the tautomer, or a pharmaceutically acceptable salt of the foregoing.

TABLE 1

| | |
|---|---|
| 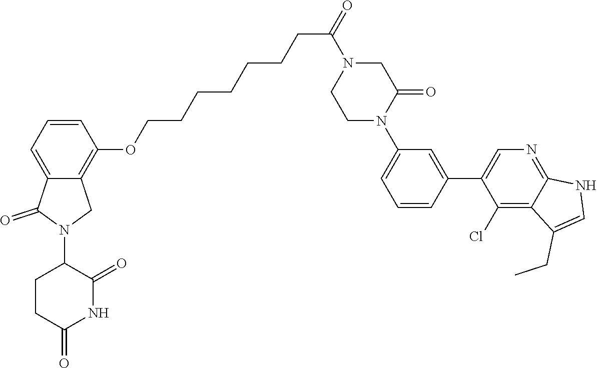 [SMILES: CCc1c[nH]c2ncc(-c3cccc(N4CCN(C(=O)CCCCCCCOc5cccc6c5CN(C5CCC(=O)NC5=O)C6=O)CC4=O)c3)c(Cl)c12] | 1 |
| 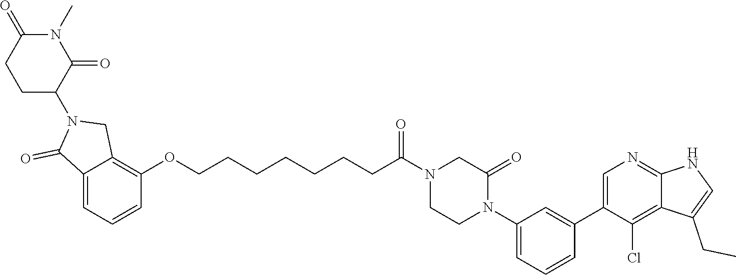 [SMILES: CCc1c[nH]c2ncc(-c3cccc(N4CCN(C(=O)CCCCCCCOc5cccc6c5CN(C5CCC(=O)N(C)C5=O)C6=O)CC4=O)c3)c(Cl)c12] | 2 |
| 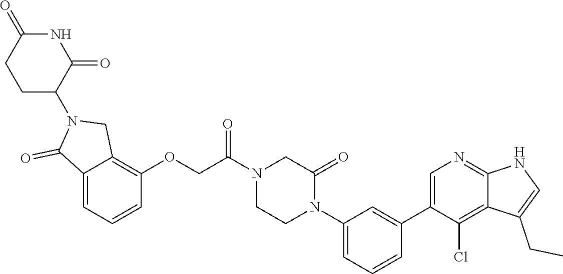 [SMILES: CCc1c[nH]c2ncc(-c3cccc(N4CCN(C(=O)COc5cccc6c5CN(C5CCC(=O)NC5=O)C6=O)CC4=O)c3)c(Cl)c12] | 3 |
| 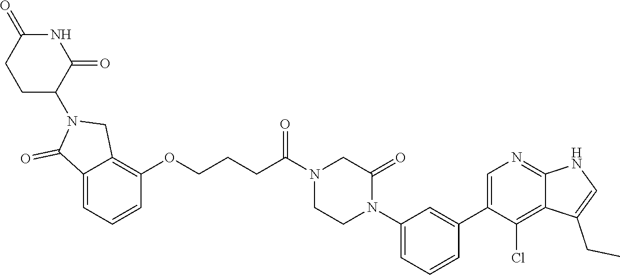 [SMILES: CCc1c[nH]c2ncc(-c3cccc(N4CCN(C(=O)CCCOc5cccc6c5CN(C5CCC(=O)NC5=O)C6=O)CC4=O)c3)c(Cl)c12] | 4 |
| 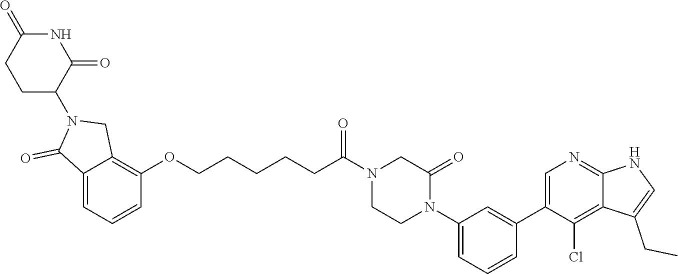 [SMILES: CCc1c[nH]c2ncc(-c3cccc(N4CCN(C(=O)CCCCCOc5cccc6c5CN(C5CCC(=O)NC5=O)C6=O)CC4=O)c3)c(Cl)c12] | 5 |
| 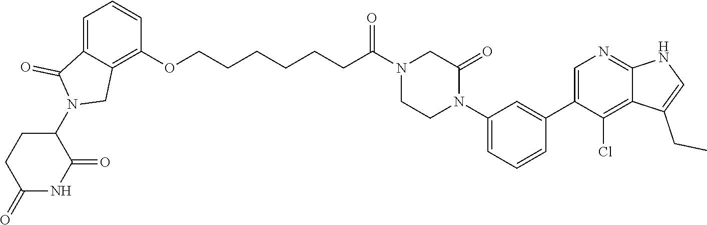 [SMILES: CCc1c[nH]c2ncc(-c3cccc(N4CCN(C(=O)CCCCCCOc5cccc6c5CN(C5CCC(=O)NC5=O)C6=O)CC4=O)c3)c(Cl)c12] | 6 |
| 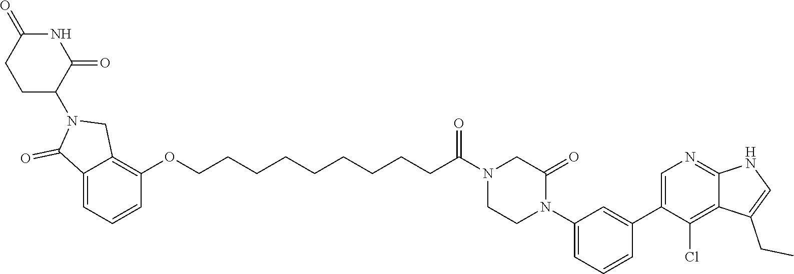 [SMILES: CCc1c[nH]c2ncc(-c3cccc(N4CCN(C(=O)CCCCCCCCCOc5cccc6c5CN(C5CCC(=O)NC5=O)C6=O)CC4=O)c3)c(Cl)c12] | 7 |
| 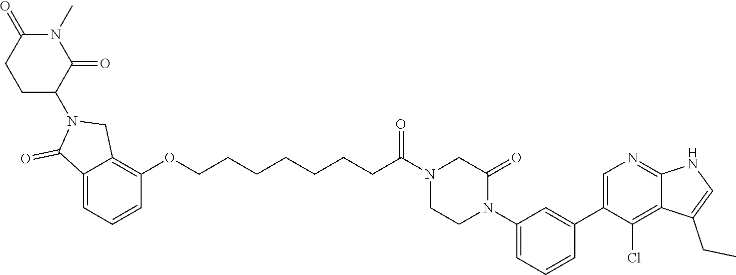 [SMILES: CCc1c[nH]c2ncc(-c3cccc(N4CCN(C(=O)CCCCCCCOc5cccc6c5CN(C5CCC(=O)N(C)C5=O)C6=O)CC4=O)c3)c(Cl)c12] | 8 |
| 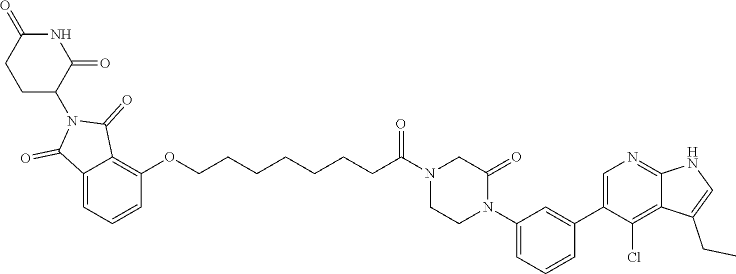 [SMILES: CCc1c[nH]c2ncc(-c3cccc(N4CCN(C(=O)CCCCCCCOc5cccc6c5C(=O)N(C5CCC(=O)NC5=O)C6=O)CC4=O)c3)c(Cl)c12] | 9 |
| 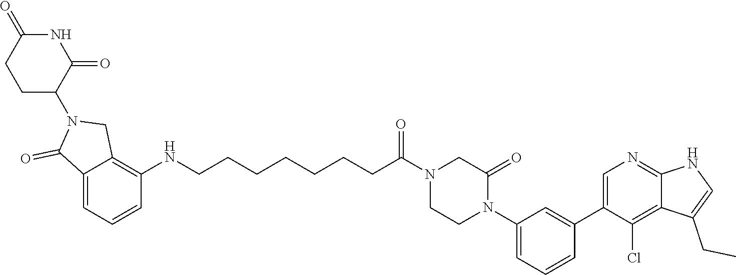 [SMILES: CCc1c[nH]c2ncc(-c3cccc(N4CCN(C(=O)CCCCCCCNc5cccc6c5CN(C5CCC(=O)NC5=O)C6=O)CC4=O)c3)c(Cl)c12] | 10 |
| 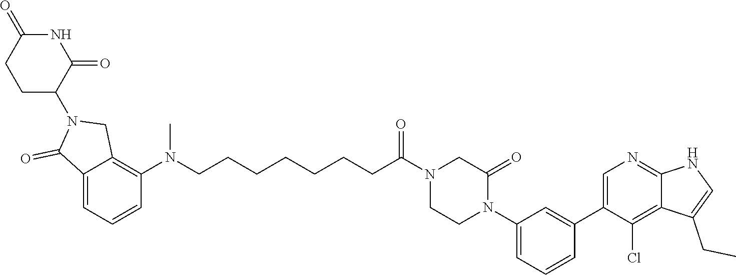 [SMILES: CCc1c[nH]c2ncc(-c3cccc(N4CCN(C(=O)CCCCCCCN(C)c5cccc6c5CN(C5CCC(=O)NC5=O)C6=O)CC4=O)c3)c(Cl)c12] | 11 |
| 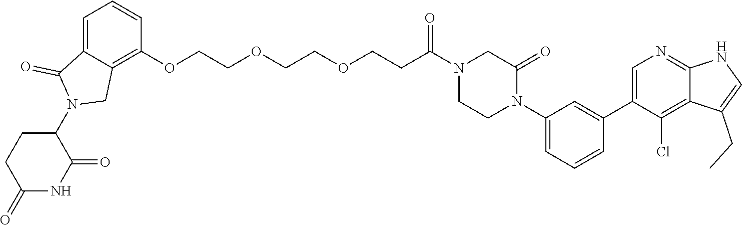 [SMILES: CCc1c[nH]c2ncc(-c3cccc(N4CCN(C(=O)CCOCCOCCOc5cccc6c5CN(C5CCC(=O)NC5=O)C6=O)CC4=O)c3)c(Cl)c12] | 12 |
| 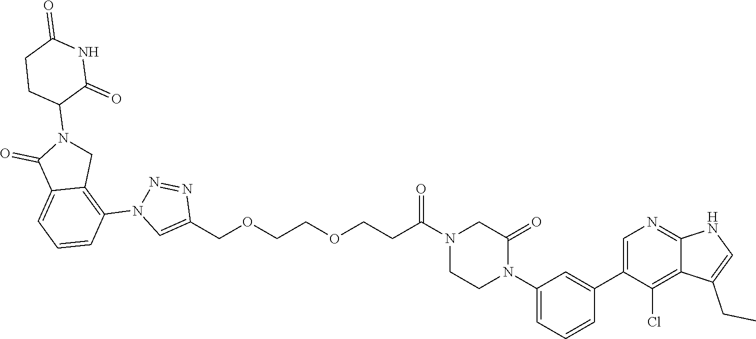 [SMILES: CCc1c[nH]c2ncc(-c3cccc(N4CCN(C(=O)CCOCCOCc5cn(-c6cccc7c6CN(C6CCC(=O)NC6=O)C7=O)nn5)CC4=O)c3)c(Cl)c12] | 13 |
| 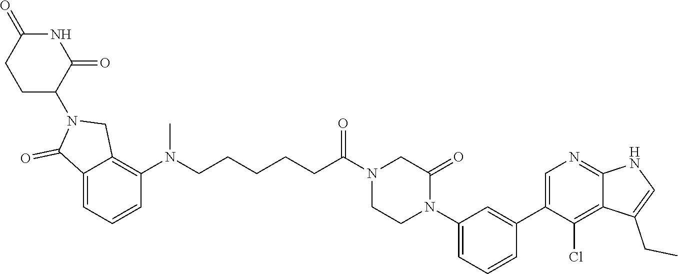 [SMILES: CCc1c[nH]c2ncc(-c3cccc(N4CCN(C(=O)CCCCCN(C)c5cccc6c5CN(C5CCC(=O)NC5=O)C6=O)CC4=O)c3)c(Cl)c12] | 14 |
| 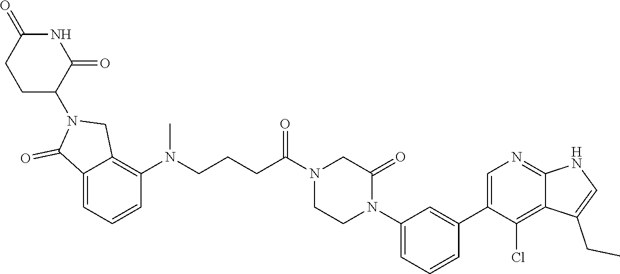 [SMILES: CCc1c[nH]c2ncc(-c3cccc(N4CCN(C(=O)CCCN(C)c5cccc6c5CN(C5CCC(=O)NC5=O)C6=O)CC4=O)c3)c(Cl)c12] | 15 |
| 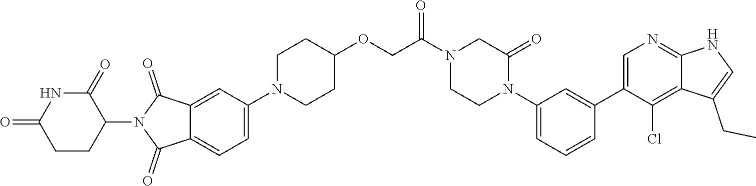 [SMILES: CCc1c[nH]c2ncc(-c3cccc(N4CCN(C(=O)COC5CCN(c6ccc7c(c6)C(=O)N(C6CCC(=O)NC6=O)C7=O)CC5)CC4=O)c3)c(Cl)c12] | 16 |
| 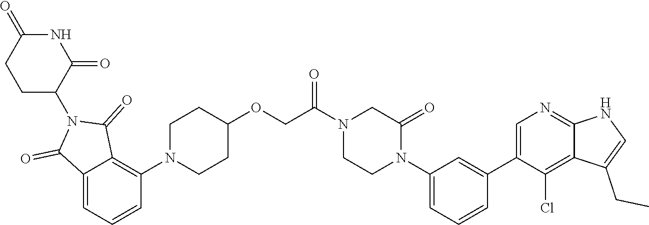 [SMILES: CCc1c[nH]c2ncc(-c3cccc(N4CCN(C(=O)COC5CCN(c6cccc7c6C(=O)N(C6CCC(=O)NC6=O)C7=O)CC5)CC4=O)c3)c(Cl)c12] | 17 |
| 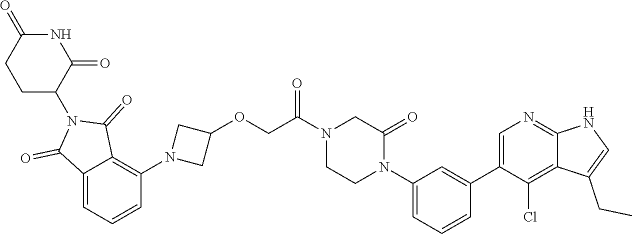 [SMILES: CCc1c[nH]c2ncc(-c3cccc(N4CCN(C(=O)COC5CN(c6cccc7c6C(=O)N(C6CCC(=O)NC6=O)C7=O)C5)CC4=O)c3)c(Cl)c12] | 18 |
| 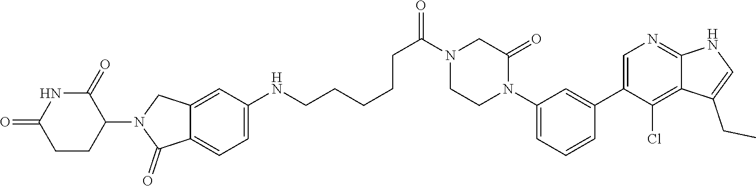 [SMILES: CCc1c[nH]c2ncc(-c3cccc(N4CCN(C(=O)CCCCCNc5ccc6c(c5)CN(C5CCC(=O)NC5=O)C6=O)CC4=O)c3)c(Cl)c12] | 19 |
| 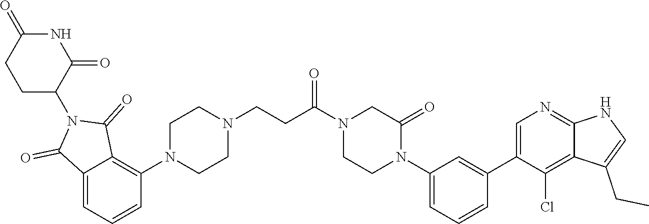 [SMILES: CCc1c[nH]c2ncc(-c3cccc(N4CCN(C(=O)CCN5CCN(c6cccc7c6C(=O)N(C6CCC(=O)NC6=O)C7=O)CC5)CC4=O)c3)c(Cl)c12] | 20 |
| 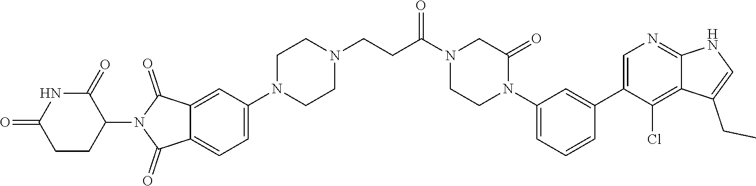 [SMILES: CCc1c[nH]c2ncc(-c3cccc(N4CCN(C(=O)CCN5CCN(c6ccc7c(c6)C(=O)N(C6CCC(=O)NC6=O)C7=O)CC5)CC4=O)c3)c(Cl)c12] | 21 |
| 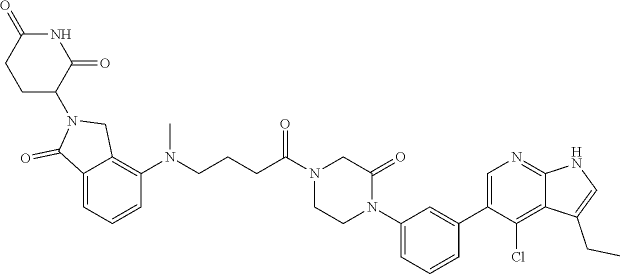 [SMILES: CCc1c[nH]c2ncc(-c3cccc(N4CCN(C(=O)CCCN(C)c5cccc6c5CN(C5CCC(=O)NC5=O)C6=O)CC4=O)c3)c(Cl)c12] | 22 |
| 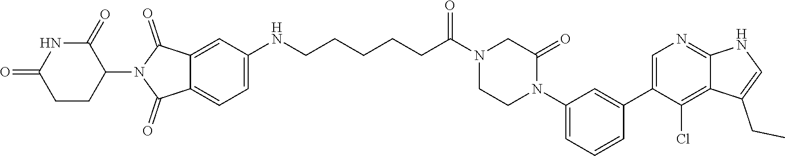 [SMILES: CCc1c[nH]c2ncc(-c3cccc(N4CCN(C(=O)CCCCCNc5ccc6c(c5)C(=O)N(C5CCC(=O)NC5=O)C6=O)CC4=O)c3)c(Cl)c12] | 23 |
| 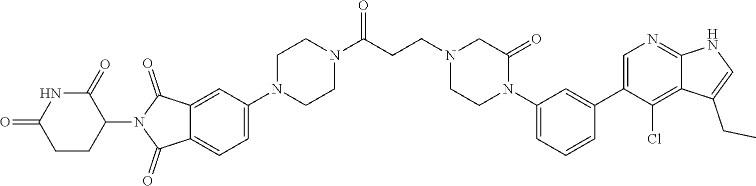 [SMILES: CCc1c[nH]c2ncc(-c3cccc(N4CCN(CCC(=O)N5CCN(c6ccc7c(c6)C(=O)N(C6CCC(=O)NC6=O)C7=O)CC5)CC4=O)c3)c(Cl)c12] | 24 |
| 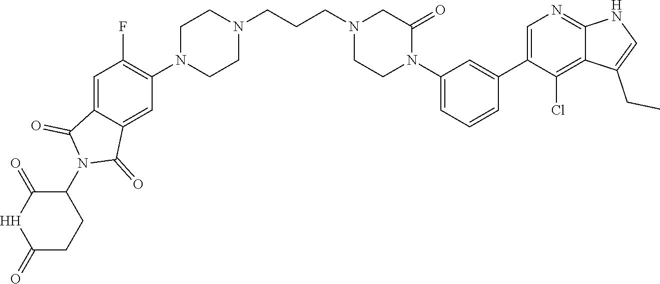 | 25 |
| 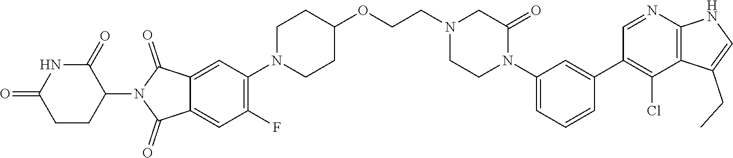 [SMILES: CCc1c[nH]c2ncc(-c3cccc(N4CCN(CCOC5CCN(c6cc7c(cc6F)C(=O)N(C6CCC(=O)NC6=O)C7=O)CC5)CC4=O)c3)c(Cl)c12] | 26 |
| 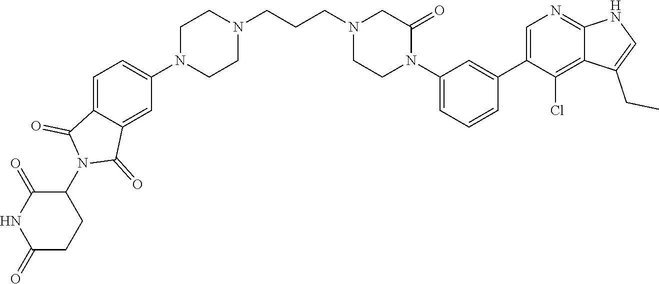 [SMILES: CCc1c[nH]c2ncc(-c3cccc(N4CCN(CCCN5CCN(c6ccc7c(c6)C(=O)N(C6CCC(=O)NC6=O)C7=O)CC5)CC4=O)c3)c(Cl)c12] | 27 |
| 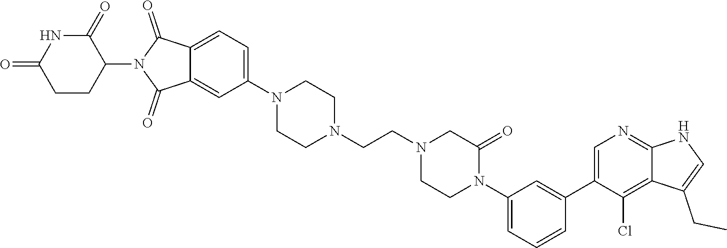 [SMILES: CCc1c[nH]c2ncc(-c3cccc(N4CCN(CCN5CCN(c6ccc7c(c6)C(=O)N(C6CCC(=O)NC6=O)C7=O)CC5)CC4=O)c3)c(Cl)c12] | 28 |
| 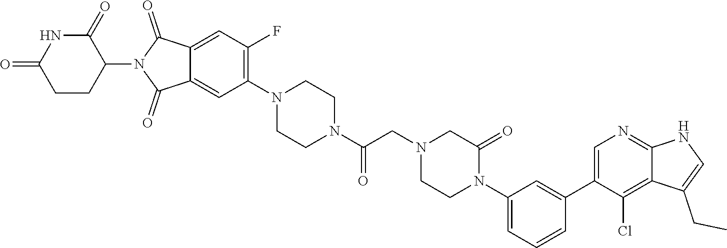 [SMILES: CCc1c[nH]c2ncc(-c3cccc(N4CCN(CC(=O)N5CCN(c6cc7c(cc6F)C(=O)N(C6CCC(=O)NC6=O)C7=O)CC5)CC4=O)c3)c(Cl)c12] | 29 |
| 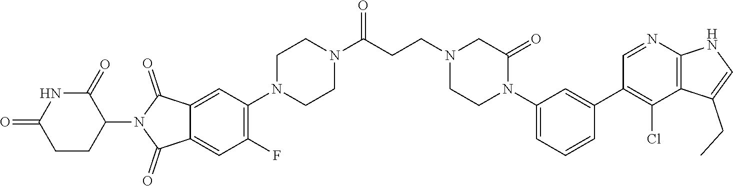 [SMILES: CCc1c[nH]c2ncc(-c3cccc(N4CCN(CCC(=O)N5CCN(c6cc7c(cc6F)C(=O)N(C6CCC(=O)NC6=O)C7=O)CC5)CC4=O)c3)c(Cl)c12] | 30 |
| 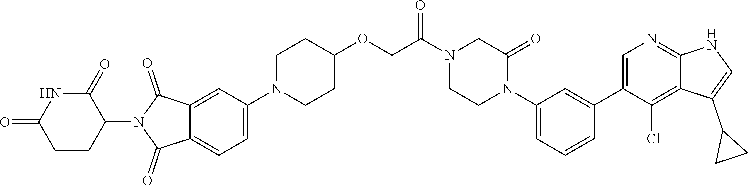 [SMILES: O=C1CCC(N2C(=O)c3ccc(N4CCC(OCC(=O)N5CCN(c6cccc(-c7cnc8[nH]cc(C9CC9)c8c7Cl)c6)C(=O)C5)CC4)cc3C2=O)C(=O)N1] | 31 |
| 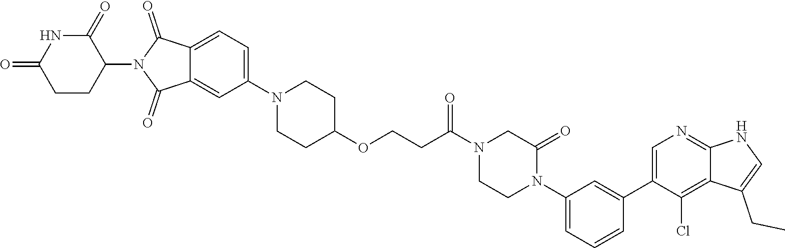 [SMILES: CCc1c[nH]c2ncc(-c3cccc(N4CCN(C(=O)CCOC5CCN(c6ccc7c(c6)C(=O)N(C6CCC(=O)NC6=O)C7=O)CC5)CC4=O)c3)c(Cl)c12] | 32 |
| 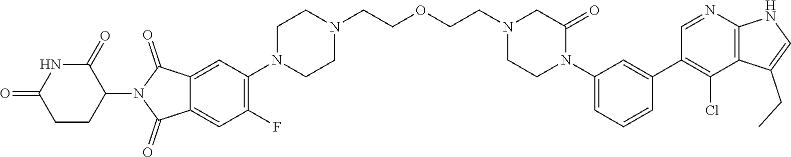 [SMILES: CCc1c[nH]c2ncc(-c3cccc(N4CCN(CCOCCN5CCN(c6cc7c(cc6F)C(=O)N(C6CCC(=O)NC6=O)C7=O)CC5)CC4=O)c3)c(Cl)c12] | 33 |
| 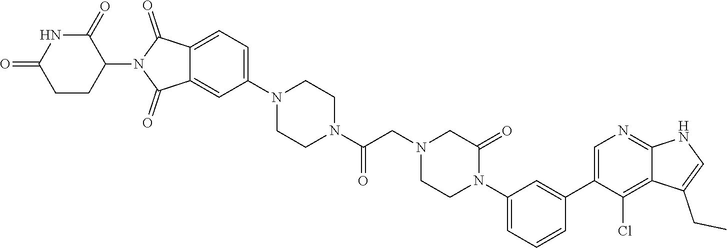 [SMILES: CCc1c[nH]c2ncc(-c3cccc(N4CCN(CC(=O)N5CCN(c6ccc7c(c6)C(=O)N(C6CCC(=O)NC6=O)C7=O)CC5)CC4=O)c3)c(Cl)c12] | 34 |
| 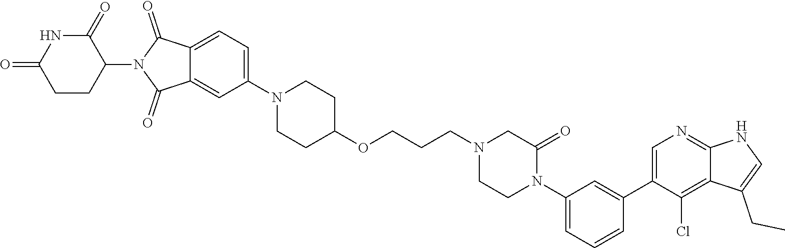 [SMILES: CCc1c[nH]c2ncc(-c3cccc(N4CCN(CCCOC5CCN(c6ccc7c(c6)C(=O)N(C6CCC(=O)NC6=O)C7=O)CC5)CC4=O)c3)c(Cl)c12] | 35 |
| 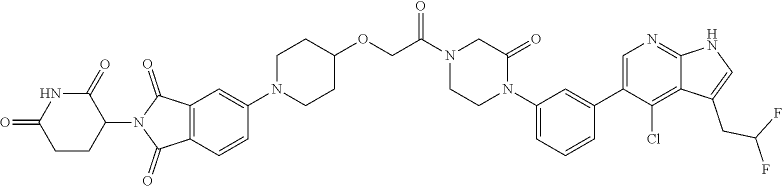 [SMILES: O=C1CCC(N2C(=O)c3ccc(N4CCC(OCC(=O)N5CCN(c6cccc(-c7cnc8[nH]cc(CC(F)F)c8c7Cl)c6)C(=O)C5)CC4)cc3C2=O)C(=O)N1] | 36 |
| 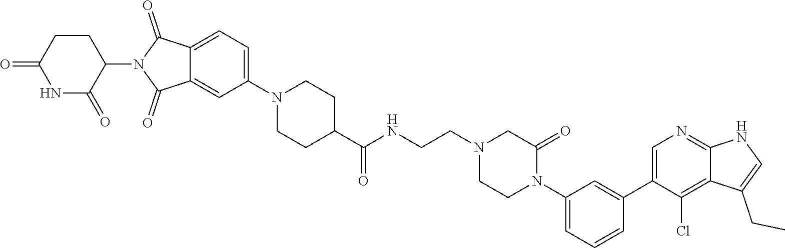 [SMILES: CCc1c[nH]c2ncc(-c3cccc(N4CCN(CCNC(=O)C5CCN(c6ccc7c(c6)C(=O)N(C6CCC(=O)NC6=O)C7=O)CC5)CC4=O)c3)c(Cl)c12] | 37 |
| 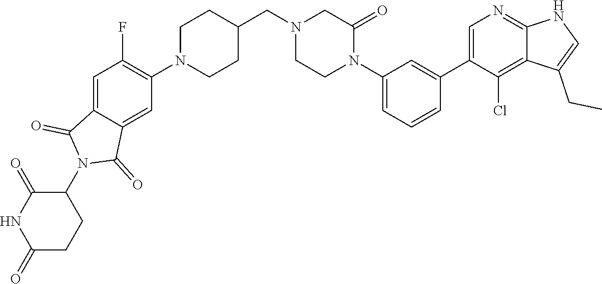 [SMILES: CCc1c[nH]c2ncc(-c3cccc(N4CCN(CC5CCN(c6cc7c(cc6F)C(=O)N(C6CCC(=O)NC6=O)C7=O)CC5)CC4=O)c3)c(Cl)c12] | 38 |
| 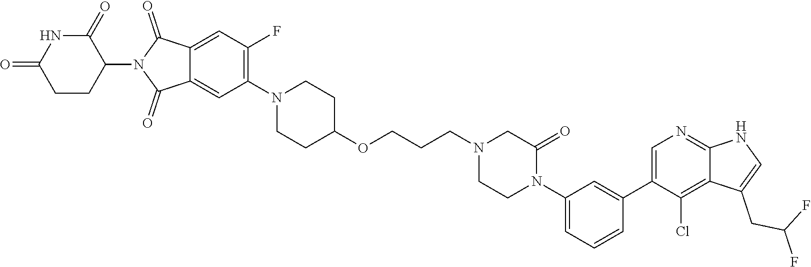 [SMILES: O=C1CCC(N2C(=O)c3cc(F)c(N4CCC(OCCCN5CCN(c6cccc(-c7cnc8[nH]cc(CC(F)F)c8c7Cl)c6)C(=O)C5)CC4)cc3C2=O)C(=O)N1] | 39 |
| 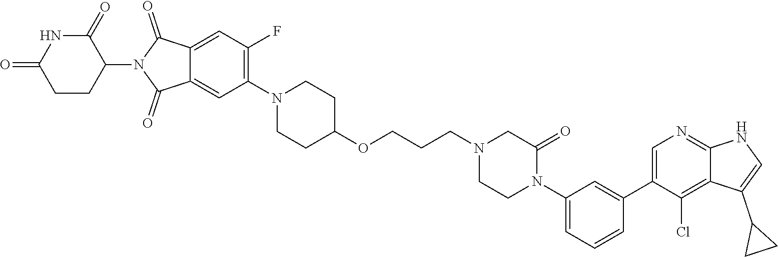 [SMILES: O=C1CCC(N2C(=O)c3cc(F)c(N4CCC(OCCCN5CCN(c6cccc(-c7cnc8[nH]cc(C9CC9)c8c7Cl)c6)C(=O)C5)CC4)cc3C2=O)C(=O)N1] | 40 |
| 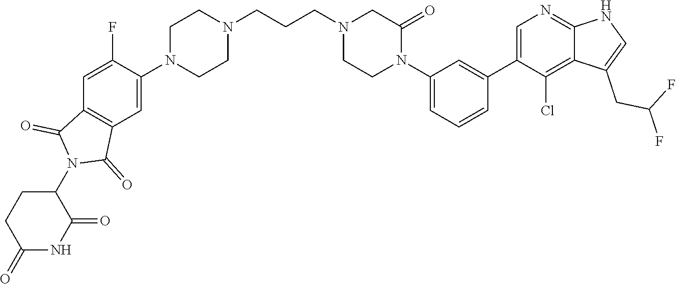 [SMILES: O=C1CCC(N2C(=O)c3cc(F)c(N4CCN(CCCN5CCN(c6cccc(-c7cnc8[nH]cc(CC(F)F)c8c7Cl)c6)C(=O)C5)CC4)cc3C2=O)C(=O)N1] | 41 |
| 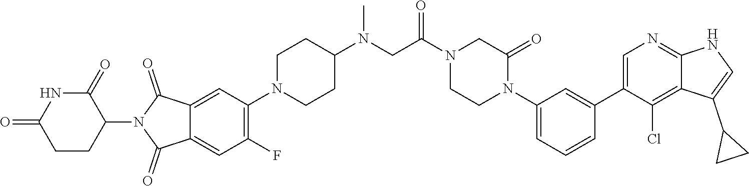 [SMILES: CN(CC(=O)N1CCN(c2cccc(-c3cnc4[nH]cc(C5CC5)c4c3Cl)c2)C(=O)C1)C1CCN(c2cc3c(cc2F)C(=O)N(C2CCC(=O)NC2=O)C3=O)CC1] | 42 |
| 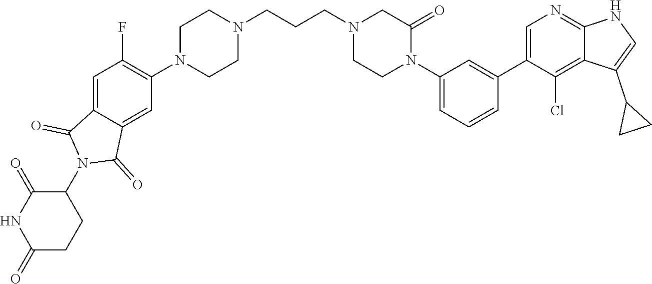 [SMILES: O=C1CCC(N2C(=O)c3cc(F)c(N4CCN(CCCN5CCN(c6cccc(-c7cnc8[nH]cc(C9CC9)c8c7Cl)c6)C(=O)C5)CC4)cc3C2=O)C(=O)N1] | 43 |
| 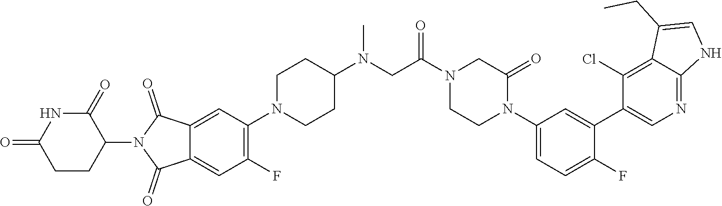 [SMILES: CCc1c[nH]c2ncc(-c3cc(N4CCN(C(=O)CN(C)C5CCN(c6cc7c(cc6F)C(=O)N(C6CCC(=O)NC6=O)C7=O)CC5)CC4=O)ccc3F)c(Cl)c12] | 44 |
| 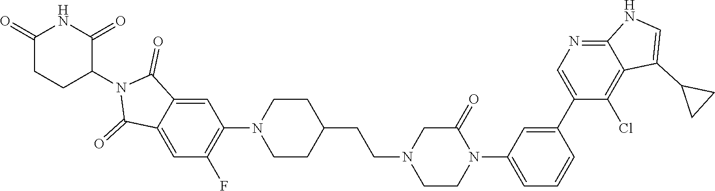 [SMILES: O=C1CCC(N2C(=O)c3cc(F)c(N4CCC(CCN5CCN(c6cccc(-c7cnc8[nH]cc(C9CC9)c8c7Cl)c6)C(=O)C5)CC4)cc3C2=O)C(=O)N1] | 45 |
| 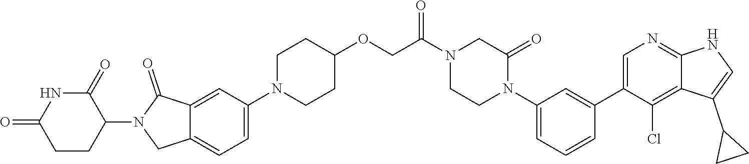 [SMILES: O=C1CCC(N2Cc3ccc(N4CCC(OCC(=O)N5CCN(c6cccc(-c7cnc8[nH]cc(C9CC9)c8c7Cl)c6)C(=O)C5)CC4)cc3C2=O)C(=O)N1] | 46 |
| 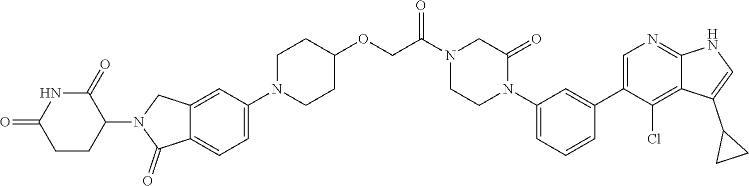 [SMILES: O=C1CCC(N2Cc3cc(N4CCC(OCC(=O)N5CCN(c6cccc(-c7cnc8[nH]cc(C9CC9)c8c7Cl)c6)C(=O)C5)CC4)ccc3C2=O)C(=O)N1] | 47 |
| 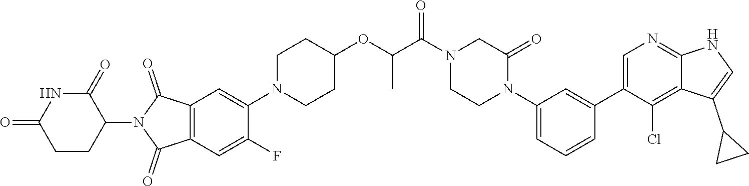 [SMILES: CC(OC1CCN(c2cc3c(cc2F)C(=O)N(C2CCC(=O)NC2=O)C3=O)CC1)C(=O)N1CCN(c2cccc(-c3cnc4[nH]cc(C5CC5)c4c3Cl)c2)C(=O)C1] | 48 |
| 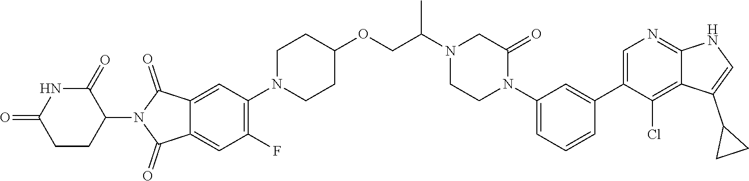 [SMILES: CC(COC1CCN(c2cc3c(cc2F)C(=O)N(C2CCC(=O)NC2=O)C3=O)CC1)N1CCN(c2cccc(-c3cnc4[nH]cc(C5CC5)c4c3Cl)c2)C(=O)C1] | 49 |
| 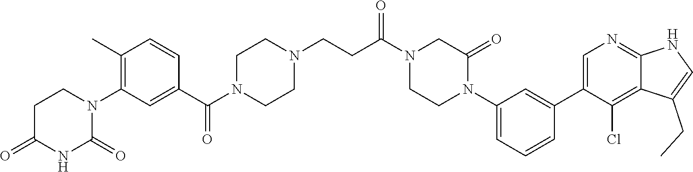 [SMILES: CCc1c[nH]c2ncc(-c3cccc(N4CCN(C(=O)CCN5CCN(C(=O)c6ccc(C)c(N7CCC(=O)NC7=O)c6)CC5)CC4=O)c3)c(Cl)c12] | 50 |
| 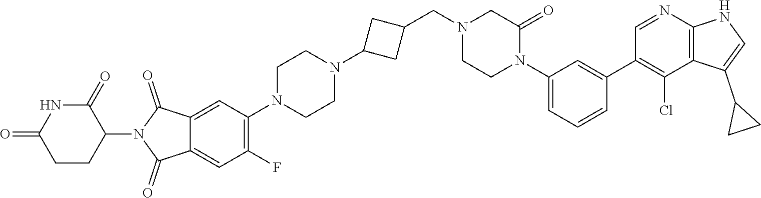 [SMILES: O=C1CCC(N2C(=O)c3cc(F)c(N4CCN(C5CC(CN6CCN(c7cccc(-c8cnc9[nH]cc(C%10CC%10)c9c8Cl)c7)C(=O)C6)C5)CC4)cc3C2=O)C(=O)N1] | 51 |
| 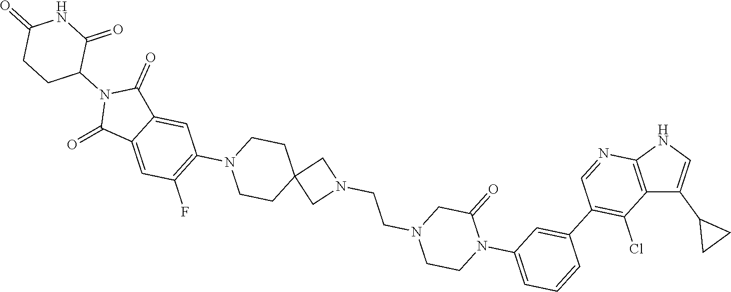 [SMILES: O=C1CCC(N2C(=O)c3cc(F)c(N4CCC5(CC4)CN(CCN4CCN(c6cccc(-c7cnc8[nH]cc(C9CC9)c8c7Cl)c6)C(=O)C4)C5)cc3C2=O)C(=O)N1] | 52 |
| 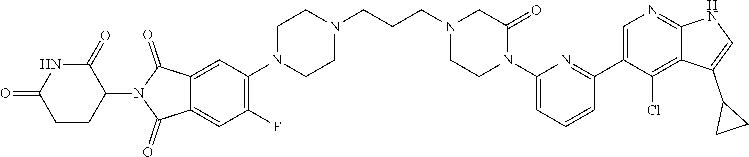 [SMILES: O=C1CCC(N2C(=O)c3cc(F)c(N4CCN(CCCN5CCN(c6cccc(-c7cnc8[nH]cc(C9CC9)c8c7Cl)n6)C(=O)C5)CC4)cc3C2=O)C(=O)N1] | 53 |
| 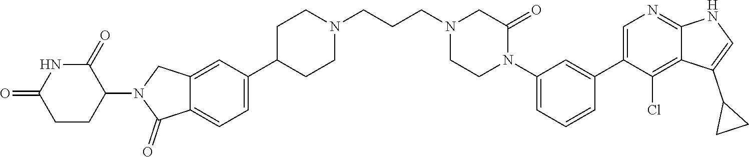 [SMILES: O=C1CCC(N2Cc3cc(C4CCN(CCCN5CCN(c6cccc(-c7cnc8[nH]cc(C9CC9)c8c7Cl)c6)C(=O)C5)CC4)ccc3C2=O)C(=O)N1] | 54 |
| 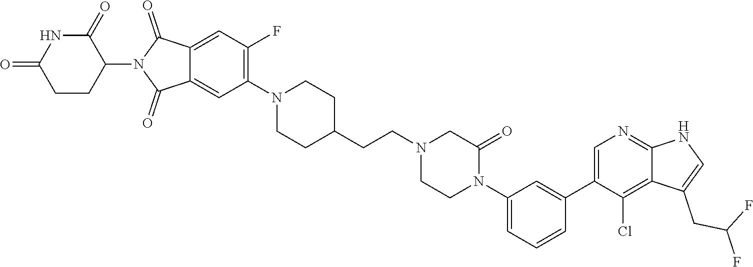 [SMILES: O=C1CCC(N2C(=O)c3cc(F)c(N4CCC(CCN5CCN(c6cccc(-c7cnc8[nH]cc(CC(F)F)c8c7Cl)c6)C(=O)C5)CC4)cc3C2=O)C(=O)N1] | 55 |
| 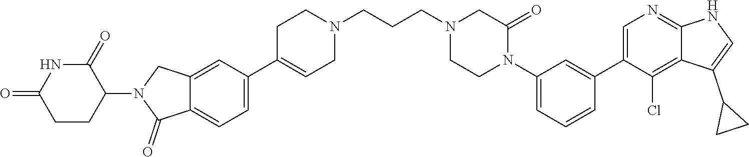 [SMILES: O=C1CCC(N2Cc3cc(C4=CCN(CCCN5CCN(c6cccc(-c7cnc8[nH]cc(C9CC9)c8c7Cl)c6)C(=O)C5)CC4)ccc3C2=O)C(=O)N1] | 56 |
| 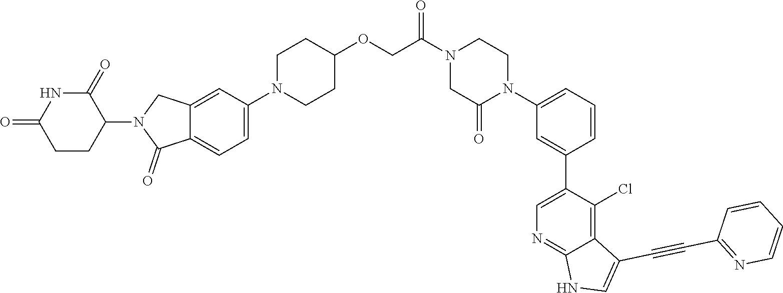 [SMILES: O=C1CCC(N2Cc3cc(N4CCC(OCC(=O)N5CCN(c6cccc(-c7cnc8[nH]cc(C#Cc9ccccn9)c8c7Cl)c6)C(=O)C5)CC4)ccc3C2=O)C(=O)N1] | 57 |
| 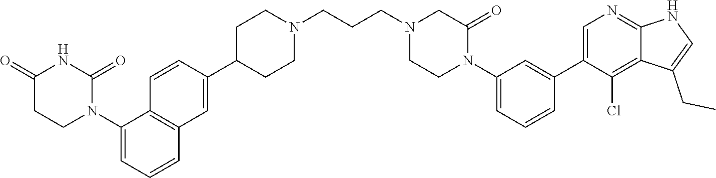 [SMILES: CCc1c[nH]c2ncc(-c3cccc(N4CCN(CCCN5CCC(c6ccc7c(N8CCC(=O)NC8=O)cccc7c6)CC5)CC4=O)c3)c(Cl)c12] | 58 |
| 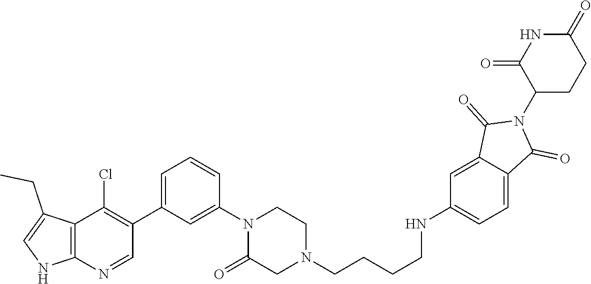 [SMILES: CCc1c[nH]c2ncc(-c3cccc(N4CCN(CCCCNc5ccc6c(c5)C(=O)N(C5CCC(=O)NC5=O)C6=O)CC4=O)c3)c(Cl)c12] | 59 |
| 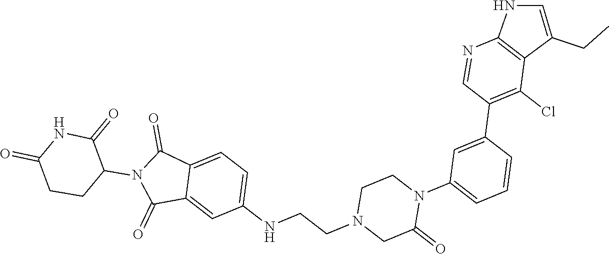 [SMILES: CCc1c[nH]c2ncc(-c3cccc(N4CCN(CCNc5ccc6c(c5)C(=O)N(C5CCC(=O)NC5=O)C6=O)CC4=O)c3)c(Cl)c12] | 60 |
| 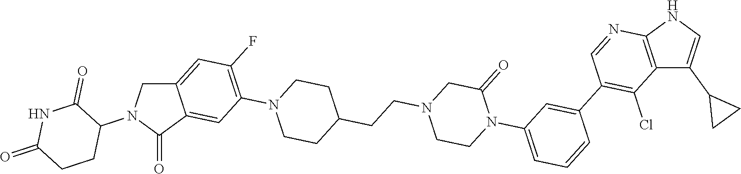 [SMILES: O=C1CCC(N2Cc3cc(F)c(N4CCC(CCN5CCN(c6cccc(-c7cnc8[nH]cc(C9CC9)c8c7Cl)c6)C(=O)C5)CC4)cc3C2=O)C(=O)N1] | 61 |
| 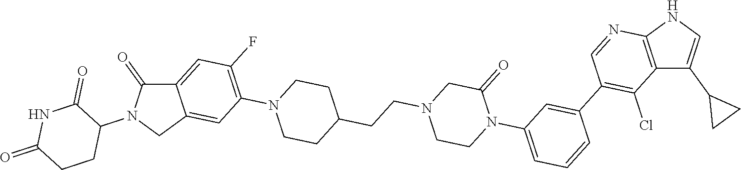 [SMILES: O=C1CCC(N2Cc3cc(N4CCC(CCN5CCN(c6cccc(-c7cnc8[nH]cc(C9CC9)c8c7Cl)c6)C(=O)C5)CC4)c(F)cc3C2=O)C(=O)N1] | 62 |
| 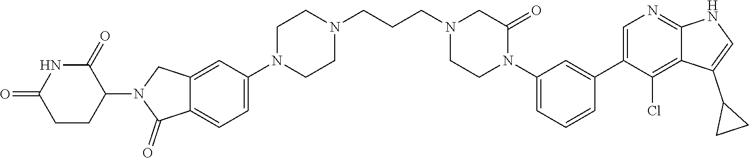 [SMILES: O=C1CCC(N2Cc3cc(N4CCN(CCCN5CCN(c6cccc(-c7cnc8[nH]cc(C9CC9)c8c7Cl)c6)C(=O)C5)CC4)ccc3C2=O)C(=O)N1] | 63 |
| 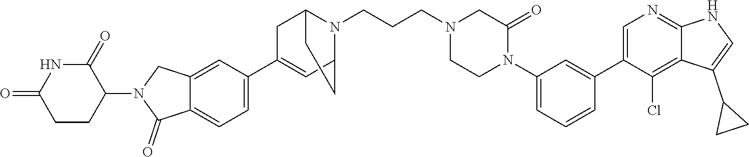 [SMILES: O=C1CCC(N2Cc3cc(C4=CC5CCC(C4)N5CCCN4CCN(c5cccc(-c6cnc7[nH]cc(C8CC8)c7c6Cl)c5)C(=O)C4)ccc3C2=O)C(=O)N1] | 64 |
| 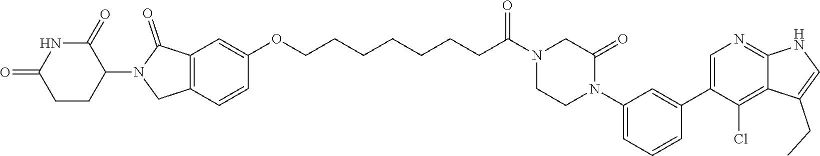 [SMILES: CCc1c[nH]c2ncc(-c3cccc(N4CCN(C(=O)CCCCCCCOc5ccc6c(c5)C(=O)N(C5CCC(=O)NC5=O)C6)CC4=O)c3)c(Cl)c12] | 65 |
| 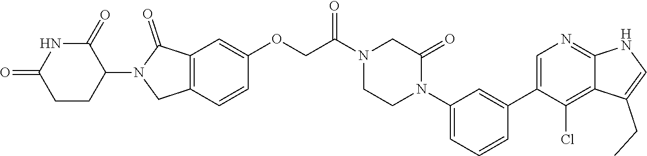 [SMILES: CCc1c[nH]c2ncc(-c3cccc(N4CCN(C(=O)COc5ccc6c(c5)C(=O)N(C5CCC(=O)NC5=O)C6)CC4=O)c3)c(Cl)c12] | 66 |
| 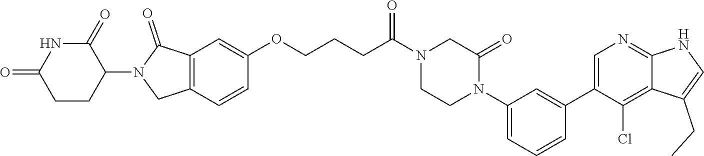 [SMILES: CCc1c[nH]c2ncc(-c3cccc(N4CCN(C(=O)CCCOc5ccc6c(c5)C(=O)N(C5CCC(=O)NC5=O)C6)CC4=O)c3)c(Cl)c12] | 67 |
| 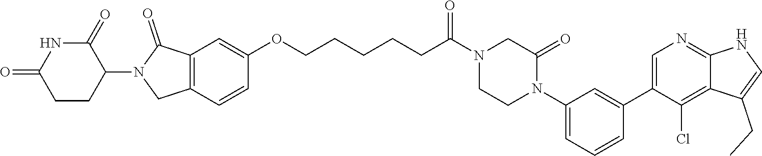 [SMILES: CCc1c[nH]c2ncc(-c3cccc(N4CCN(C(=O)CCCCCOc5ccc6c(c5)C(=O)N(C5CCC(=O)NC5=O)C6)CC4=O)c3)c(Cl)c12] | 68 |
| 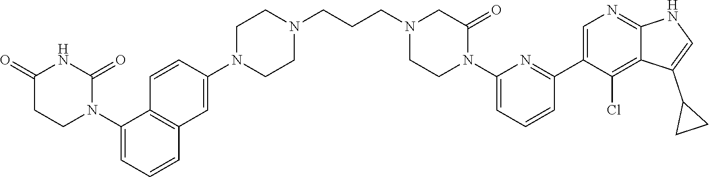 [SMILES: O=C1CCN(c2cccc3cc(N4CCN(CCCN5CCN(c6cccc(-c7cnc8[nH]cc(C9CC9)c8c7Cl)n6)C(=O)C5)CC4)ccc23)C(=O)N1] | 69 |
| 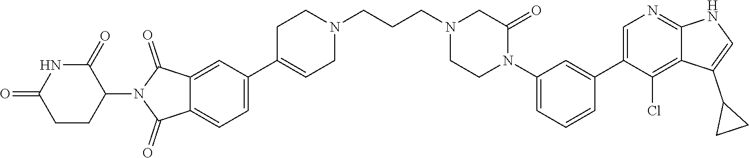 [SMILES: O=C1CCC(N2C(=O)c3ccc(C4=CCN(CCCN5CCN(c6cccc(-c7cnc8[nH]cc(C9CC9)c8c7Cl)c6)C(=O)C5)CC4)cc3C2=O)C(=O)N1] | 70 |
| 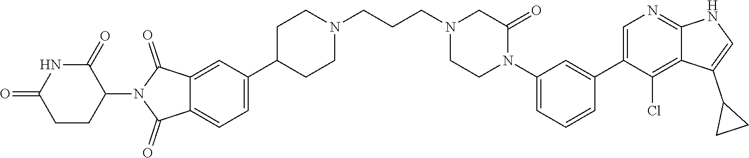 [SMILES: O=C1CCC(N2C(=O)c3ccc(C4CCN(CCCN5CCN(c6cccc(-c7cnc8[nH]cc(C9CC9)c8c7Cl)c6)C(=O)C5)CC4)cc3C2=O)C(=O)N1] | 71 |
| 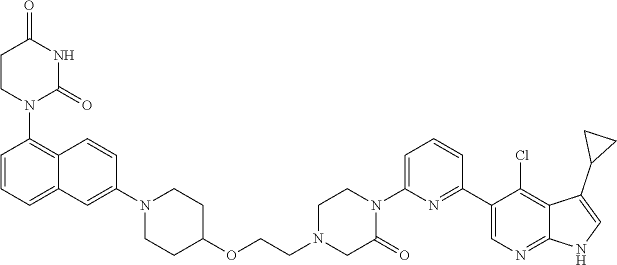 [SMILES: O=C1CCN(c2cccc3cc(N4CCC(OCCN5CCN(c6cccc(-c7cnc8[nH]cc(C9CC9)c8c7Cl)n6)C(=O)C5)CC4)ccc23)C(=O)N1] | 72 |
| 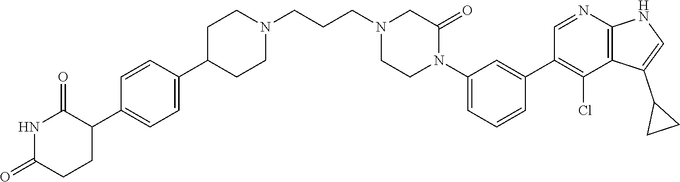 [SMILES: O=C1CCC(c2ccc(C3CCN(CCCN4CCN(c5cccc(-c6cnc7[nH]cc(C8CC8)c7c6Cl)c5)C(=O)C4)CC3)cc2)C(=O)N1] | 73 |
| 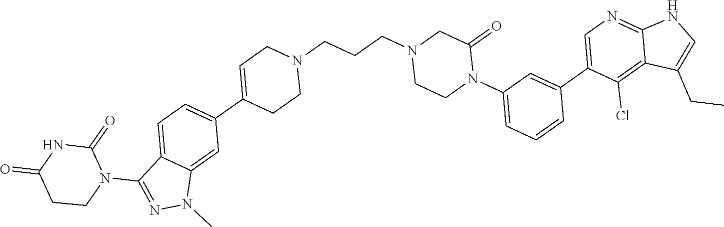 [SMILES: CCc1c[nH]c2ncc(-c3cccc(N4CCN(CCCN5CC=C(c6ccc7c(N8CCC(=O)NC8=O)nn(C)c7c6)CC5)CC4=O)c3)c(Cl)c12] | 74 |
| 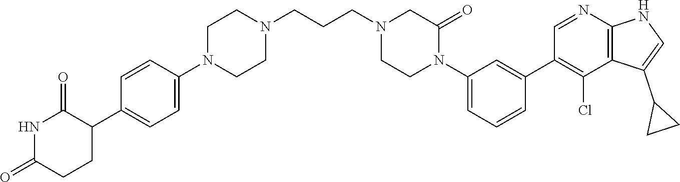 [SMILES: O=C1CCC(c2ccc(N3CCN(CCCN4CCN(c5cccc(-c6cnc7[nH]cc(C8CC8)c7c6Cl)c5)C(=O)C4)CC3)cc2)C(=O)N1] | 75 |
| 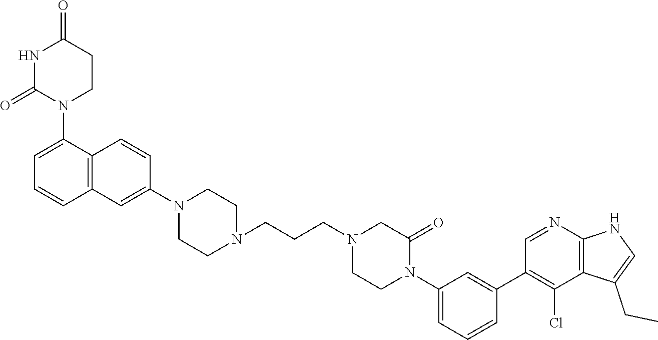 [SMILES: CCc1c[nH]c2ncc(-c3cccc(N4CCN(CCCN5CCN(c6ccc7c(N8CCC(=O)NC8=O)cccc7c6)CC5)CC4=O)c3)c(Cl)c12] | 76 |
| 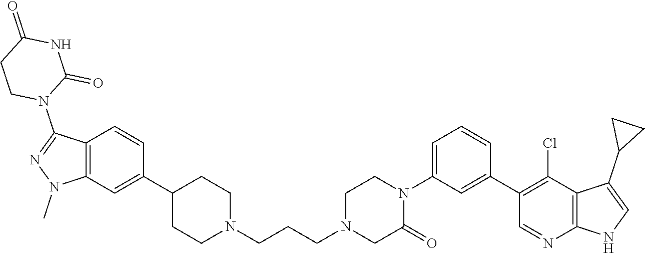 [SMILES: Cn1nc(N2CCC(=O)NC2=O)c2ccc(C3CCN(CCCN4CCN(c5cccc(-c6cnc7[nH]cc(C8CC8)c7c6Cl)c5)C(=O)C4)CC3)cc21] | 77 |
| 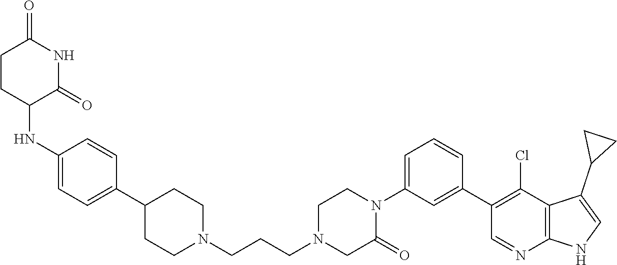 [SMILES: O=C1CCC(Nc2ccc(C3CCN(CCCN4CCN(c5cccc(-c6cnc7[nH]cc(C8CC8)c7c6Cl)c5)C(=O)C4)CC3)cc2)C(=O)N1] | 78 |
| 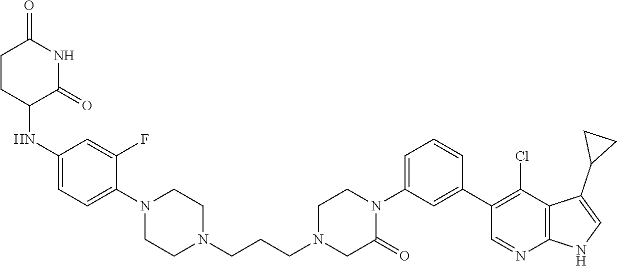 [SMILES: O=C1CCC(Nc2ccc(N3CCN(CCCN4CCN(c5cccc(-c6cnc7[nH]cc(C8CC8)c7c6Cl)c5)C(=O)C4)CC3)c(F)c2)C(=O)N1] | 79 |
| 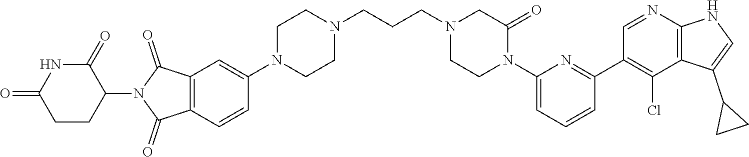 [SMILES: O=C1CCC(N2C(=O)c3ccc(N4CCN(CCCN5CCN(c6cccc(-c7cnc8[nH]cc(C9CC9)c8c7Cl)n6)C(=O)C5)CC4)cc3C2=O)C(=O)N1] | 80 |
| 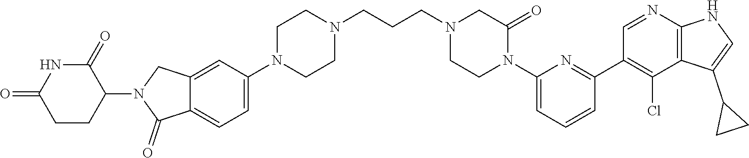 [SMILES: O=C1CCC(N2Cc3cc(N4CCN(CCCN5CCN(c6cccc(-c7cnc8[nH]cc(C9CC9)c8c7Cl)n6)C(=O)C5)CC4)ccc3C2=O)C(=O)N1] | 81 |
| 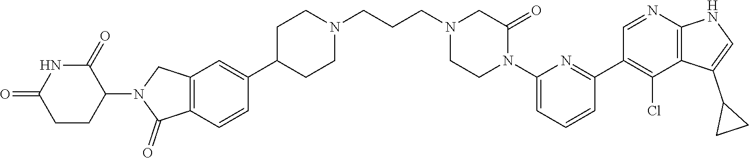 [SMILES: O=C1CCC(N2Cc3cc(C4CCN(CCCN5CCN(c6cccc(-c7cnc8[nH]cc(C9CC9)c8c7Cl)n6)C(=O)C5)CC4)ccc3C2=O)C(=O)N1] | 82 |
| 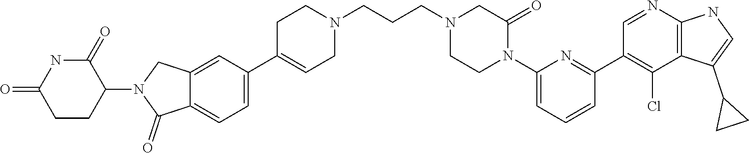 [SMILES: O=C1CCC(N2Cc3cc(C4=CCN(CCCN5CCN(c6cccc(-c7cnc8[nH]cc(C9CC9)c8c7Cl)n6)C(=O)C5)CC4)ccc3C2=O)C(=O)N1] | 83 |
| 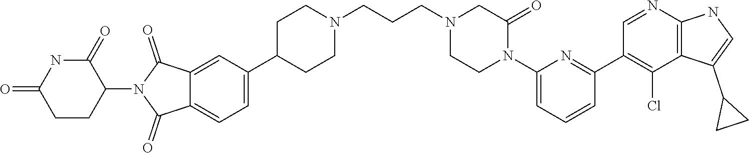 [SMILES: O=C1CCC(N2C(=O)c3ccc(C4CCN(CCCN5CCN(c6cccc(-c7cnc8[nH]cc(C9CC9)c8c7Cl)n6)C(=O)C5)CC4)cc3C2=O)C(=O)N1] | 84 |
| 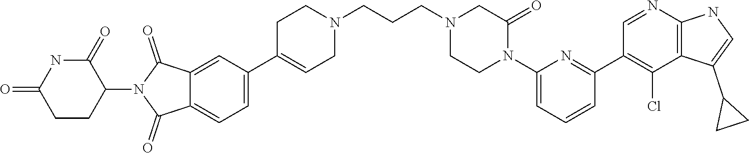 [SMILES: O=C1CCC(N2C(=O)c3ccc(C4=CCN(CCCN5CCN(c6cccc(-c7cnc8[nH]cc(C9CC9)c8c7Cl)n6)C(=O)C5)CC4)cc3C2=O)C(=O)N1] | 85 |
| 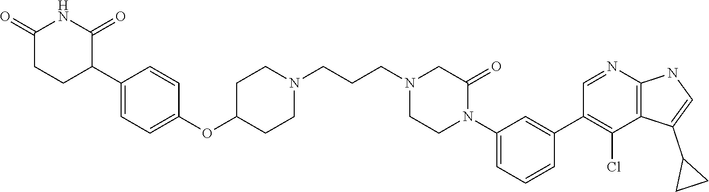 [SMILES: O=C1CCC(c2ccc(OC3CCN(CCCN4CCN(c5cccc(-c6cnc7[nH]cc(C8CC8)c7c6Cl)c5)C(=O)C4)CC3)cc2)C(=O)N1] | 86 |
| 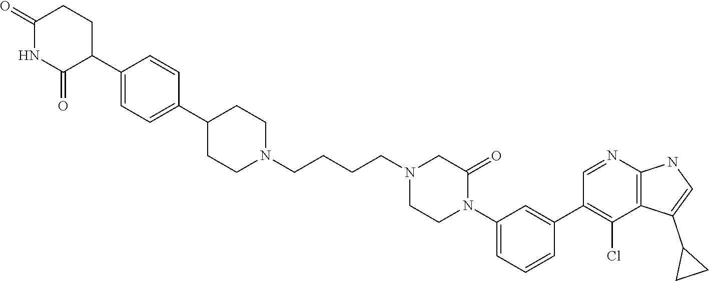 [SMILES: O=C1CCC(c2ccc(C3CCN(CCCCN4CCN(c5cccc(-c6cnc7[nH]cc(C8CC8)c7c6Cl)c5)C(=O)C4)CC3)cc2)C(=O)N1] | 87 |
| 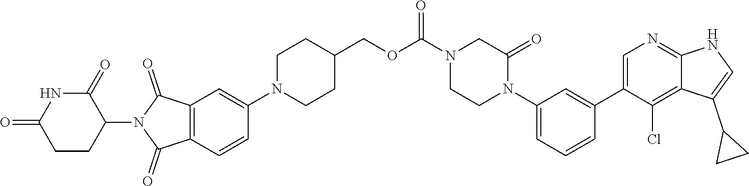 [SMILES: O=C1CCC(N2C(=O)c3ccc(N4CCC(COC(=O)N5CCN(c6cccc(-c7cnc8[nH]cc(C9CC9)c8c7Cl)c6)C(=O)C5)CC4)cc3C2=O)C(=O)N1] | 88 |
| 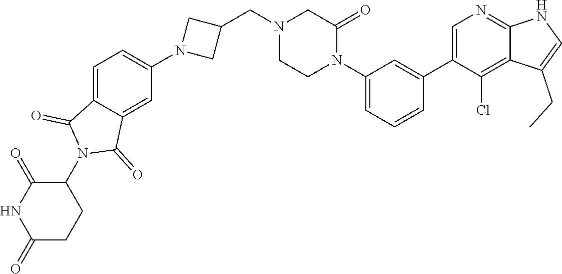 [SMILES: CCc1c[nH]c2ncc(-c3cccc(N4CCN(CC5CN(c6ccc7c(c6)C(=O)N(C6CCC(=O)NC6=O)C7=O)C5)CC4=O)c3)c(Cl)c12] | 89 |
| 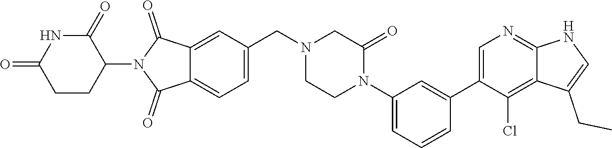 [SMILES: CCc1c[nH]c2ncc(-c3cccc(N4CCN(Cc5ccc6c(c5)C(=O)N(C5CCC(=O)NC5=O)C6=O)CC4=O)c3)c(Cl)c12] | 90 |
| 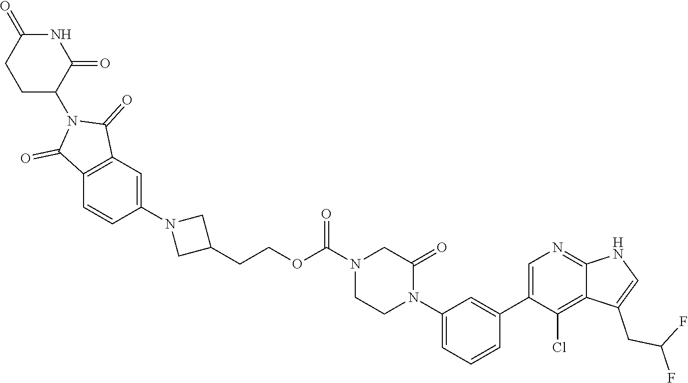 [SMILES: O=C1CCC(N2C(=O)c3ccc(N4CC(CCOC(=O)N5CCN(c6cccc(-c7cnc8[nH]cc(CC(F)F)c8c7Cl)c6)C(=O)C5)C4)cc3C2=O)C(=O)N1] | 91 |
| 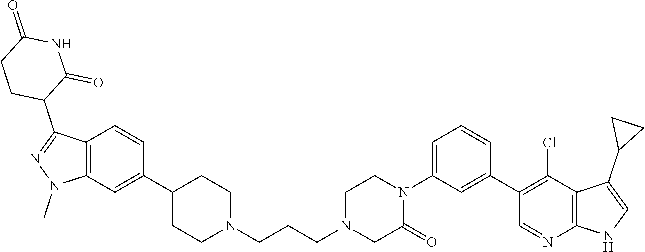 [SMILES: Cn1nc(C2CCC(=O)NC2=O)c2ccc(C3CCN(CCCN4CCN(c5cccc(-c6cnc7[nH]cc(C8CC8)c7c6Cl)c5)C(=O)C4)CC3)cc21] | 92 |
| 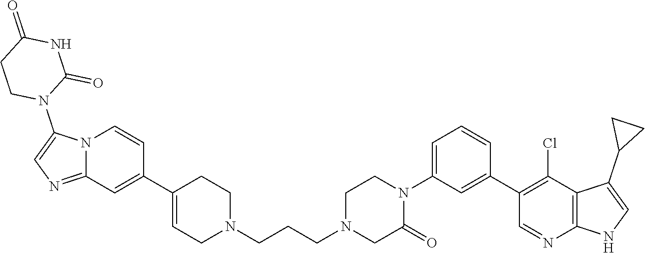 [SMILES: O=C1CCN(c2cnc3cc(C4=CCN(CCCN5CCN(c6cccc(-c7cnc8[nH]cc(C9CC9)c8c7Cl)c6)C(=O)C5)CC4)ccn23)C(=O)N1] | 93 |
| 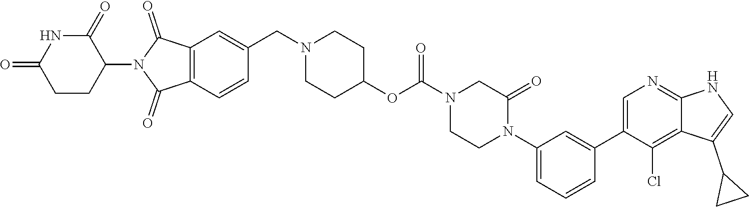 [SMILES: O=C1CCC(N2C(=O)c3ccc(CN4CCC(OC(=O)N5CCN(c6cccc(-c7cnc8[nH]cc(C9CC9)c8c7Cl)c6)C(=O)C5)CC4)cc3C2=O)C(=O)N1] | 94 |
| 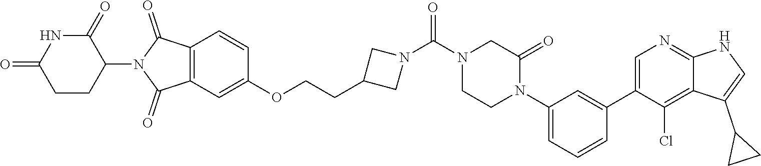 [SMILES: O=C1CCC(N2C(=O)c3ccc(OCCC4CN(C(=O)N5CCN(c6cccc(-c7cnc8[nH]cc(C9CC9)c8c7Cl)c6)C(=O)C5)C4)cc3C2=O)C(=O)N1] | 95 |
| 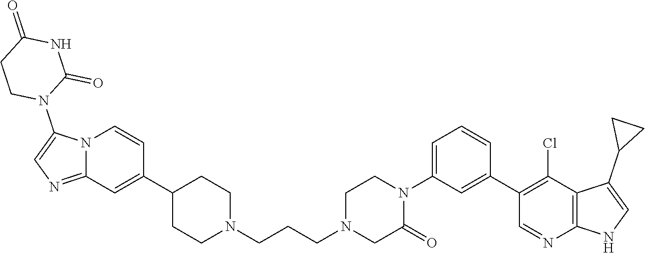 [SMILES: O=C1CCN(c2cnc3cc(C4CCN(CCCN5CCN(c6cccc(-c7cnc8[nH]cc(C9CC9)c8c7Cl)c6)C(=O)C5)CC4)ccn23)C(=O)N1] | 96 |
| 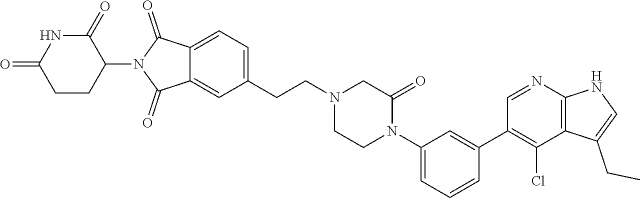 [SMILES: CCc1c[nH]c2ncc(-c3cccc(N4CCN(CCc5ccc6c(c5)C(=O)N(C5CCC(=O)NC5=O)C6=O)CC4=O)c3)c(Cl)c12] | 97 |
| 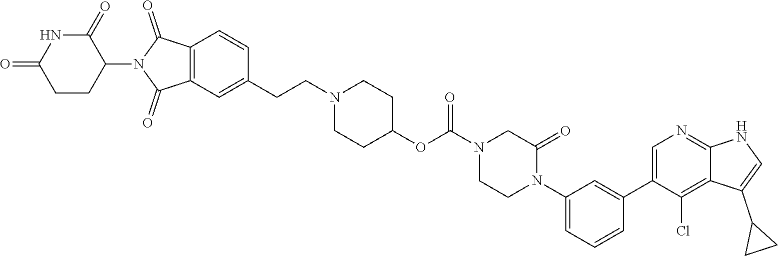 [SMILES: O=C1CCC(N2C(=O)c3ccc(CCN4CCC(OC(=O)N5CCN(c6cccc(-c7cnc8[nH]cc(C9CC9)c8c7Cl)c6)C(=O)C5)CC4)cc3C2=O)C(=O)N1] | 98 |
| 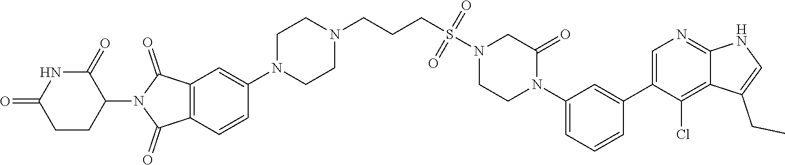 [SMILES: CCc1c[nH]c2ncc(-c3cccc(N4CCN(S(=O)(=O)CCCN5CCN(c6ccc7c(c6)C(=O)N(C6CCC(=O)NC6=O)C7=O)CC5)CC4=O)c3)c(Cl)c12] | 99 |
| 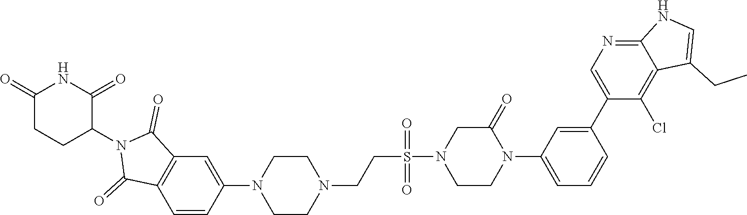 [SMILES: CCc1c[nH]c2ncc(-c3cccc(N4CCN(S(=O)(=O)CCN5CCN(c6ccc7c(c6)C(=O)N(C6CCC(=O)NC6=O)C7=O)CC5)CC4=O)c3)c(Cl)c12] | 100 |
| 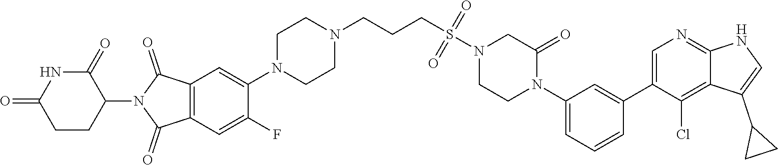 [SMILES: O=C1CCC(N2C(=O)c3cc(F)c(N4CCN(CCCS(=O)(=O)N5CCN(c6cccc(-c7cnc8[nH]cc(C9CC9)c8c7Cl)c6)C(=O)C5)CC4)cc3C2=O)C(=O)N1] | 101 |
| 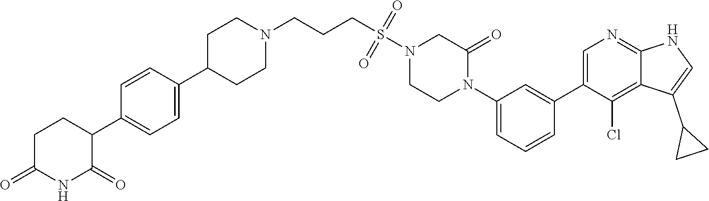 [SMILES: O=C1CCC(c2ccc(C3CCN(CCCS(=O)(=O)N4CCN(c5cccc(-c6cnc7[nH]cc(C8CC8)c7c6Cl)c5)C(=O)C4)CC3)cc2)C(=O)N1] | 102 |
| 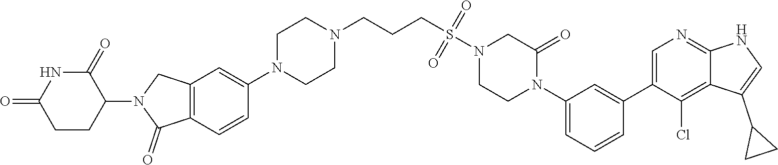 [SMILES: O=C1CCC(N2Cc3cc(N4CCN(CCCS(=O)(=O)N5CCN(c6cccc(-c7cnc8[nH]cc(C9CC9)c8c7Cl)c6)C(=O)C5)CC4)ccc3C2=O)C(=O)N1] | 103 |
| 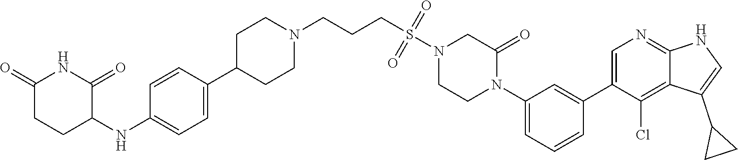 [SMILES: O=C1CCC(Nc2ccc(C3CCN(CCCS(=O)(=O)N4CCN(c5cccc(-c6cnc7[nH]cc(C8CC8)c7c6Cl)c5)C(=O)C4)CC3)cc2)C(=O)N1] | 104 |
| 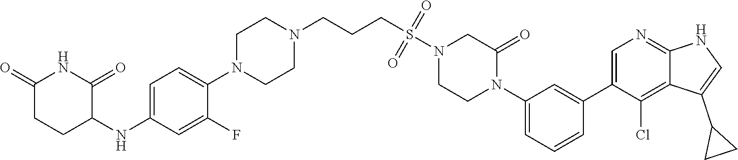 [SMILES: O=C1CCC(Nc2ccc(N3CCN(CCCS(=O)(=O)N4CCN(c5cccc(-c6cnc7[nH]cc(C8CC8)c7c6Cl)c5)C(=O)C4)CC3)c(F)c2)C(=O)N1] | 105 |
| 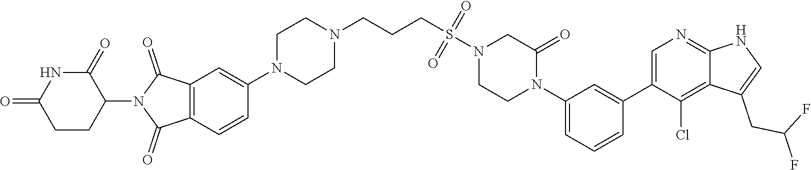 [SMILES: O=C1CCC(N2C(=O)c3ccc(N4CCN(CCCS(=O)(=O)N5CCN(c6cccc(-c7cnc8[nH]cc(CC(F)F)c8c7Cl)c6)C(=O)C5)CC4)cc3C2=O)C(=O)N1] | 106 |

Another aspect of the present disclosure provides pharmaceutical compositions comprising at least one compound selected from a compound of Formulae I and I′, Compounds 1 to 106, a tautomer thereof, a deuterated derivative of the compound or the tautomer, or a pharmaceutically acceptable salt of the foregoing, or a pharmaceutical composition comprising any of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of the present disclosure can be employed in combination therapies; that is, the pharmaceutical compositions disclosed herein can further include an additional active pharmaceutical agent. Alternatively, a pharmaceutical composition comprising a compound selected from a compound of Formulae I and I′, Compounds 1 to 106, a tautomer thereof, a deuterated derivative of the compound or the tautomer, or a pharmaceutically acceptable salt of the foregoing, or a pharmaceutical composition comprising any of the foregoing can be administered as a separate composition concurrently with, prior to, or subsequent to, a composition comprising an additional active pharmaceutical agent.

As discussed above, the pharmaceutical compositions disclosed herein comprise a pharmaceutically acceptable carrier. The pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of the present disclosure, such as by producing any undesirable biological effect or otherwise interacting in a deleterious manner with any other component(s) of the pharmaceutical composition, its use is contemplated to be within the scope of the present disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### III. Methods of Treatment and Uses

In another aspect of the present disclosure, a compound, tautomer, deuterated derivative, or pharmaceutically acceptable salt as disclosed herein, including a compound of Formulae I and I′, Compounds 1 to 106, a tautomer thereof, a deuterated derivative of the compound or the tautomer, or a pharmaceutically acceptable salt of the foregoing, or the pharmaceutical composition thereof, is for use in treating a disease, a disorder, or a condition mediated by the degradation of HPK1. In another aspect, disclosed herein is use of a compound, tautomer, deuterated derivative, or pharmaceutically acceptable salt as disclosed herein, including a compound of Formulae I and I′, Compounds 1 to 106, a tautomer thereof, a deuterated derivative of the compound or the tautomer, or a pharmaceutically acceptable salt of the foregoing, or the pharmaceutical composition thereof, for the manufacture of a medicament for treating a disease, a disorder, or a condition mediated by the degradation of HPK1. In yet another aspect, disclosed herein is a method of treating a disease, a disorder, or a condition mediated by the degradation of HPK1 in a subject, comprising administering a therapeutically effective amount of a compound, tautomer, deuterated derivative, or pharmaceutically acceptable salt as disclosed herein, including a compound of Formulae I and I′, Compounds 1 to 106, a tautomer thereof, a deuterated derivative of the compound or the tautomer, or a pharmaceutically acceptable salt of the foregoing, or the pharmaceutical composition thereof.

In some embodiments, the disease, the disorder, or the condition is chosen from an HPK1-related disease. In some embodiments, the disease, the disorder, or the condition is selected from cancer, a dysregulated immune response, or a disease involved in aberrant HPK1 expression, activity, and/or signaling. In some embodiments, the cancer is chosen from brain cancer, breast cancer, respiratory tract and/or lung cancer, a reproductive organ cancer, bone cancer, digestive tract cancer, urinary tract cancer, eye cancer, liver cancer, kidney cancer, skin cancer, head and neck cancer, anal cancer, nervous system cancer, thyroid cancer, parathyroid cancer, a lymphoma, a sarcoma, and a leukemia.

In some embodiments, the brain cancer is chosen from brain stem and hypothalamic glioma, cerebellar and cerebral astrocytoma, glioblastoma_multiforme, medulloblastoma, ependymoma, neuroectodermal, and pineal tumor. In some embodiments, the liver cancer is chosen from hepatocellular carcinoma (liver cell carcinomas with or without fibrolamellar variant), cholangiocarcinoma (intrahepatic bile duct carcinoma) and mixed hepatocellular cholangiocarcinoma. In some embodiments, the respiratory tract and/or lung cancer is chosen from small cell lung cancer, non-small cell lung cancer, bronchial adenoma, and pleuropulmonary blastoma. In some embodiments, the digestive tract cancer is chosen from anal, colon, rectal, gallbladder, gastric, esophagus cancer, stomach, pancreas, salivary gland, small-intestine and colorectal cancer. In some embodiments, the kidney cancer is chosen from renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor. In some embodiments, the skin cancer is chosen from malignant melanoma, squamous cell carcinoma, Kaposi's sarcoma, Merkel cell skin cancer and non-melanoma skin cancer. In some embodiments, the head and neck cancer is chosen from squamous cell cancer of the head and neck, laryngeal hypopharyngeal, nasopharyngeal, oropharyngeal cancer, nasal and paranasal cancers, salivary gland cancer, lip and oral cavity cancer and squamous cell. In some embodiments, the reproductive organ cancer is chosen from prostate cancer, testicular cancer, endometrial cancer, cervical cancer, ovarian cancer, vaginal cancer, vulvar cancer, and uterus sarcoma. In some embodiments, the ovarian cancer is chosen from serous tumor, endometrioid tumor, mucinous cystadenocarcinoma, granulasa cell tumor, Sertoli-Leydig cell tumor, and arrhenoblastoma. In some embodiments, the cervical cancer is chosen from squamous cell carcinoma, adenocarcinoma, adenosquamous carcinoma, small cell carcinoma, neuroendocrine tumor, glassy cell carcinoma, and villogladular adenocarcinoma. In some embodiments, the bone cancer is chosen from osteogenic sarcoma, fibrosarcoma, malignant fibrous histiocytoma, chondrosarcoma, Ewing's sarcoma, malignant lymphoma, multiple myeloma, malignant giant cell tumor chordoma, osteochondroma, benign chondroma, chondroblastoma, chondromyxofibroma, osteoid osteoma, and giant cell tumor. In some embodiments, the breast cancer is chosen from triple negative breast cancer, invasive ductal carcinoma, invasive lobular carcinoma, ductal carcinoma in situ, and lobular carcinoma in situ. In some embodiments, the sarcoma is chosen from sarcoma of the soft tissue, chondrosarcoma, Ewing's sarcoma, angiosarcoma, fibrosarcoma, myxoma, rhabdomyoma, fibroma, lipoma, harmatoma, teratoma, osteosarcoma, malignant fibrous histiocytoma, liposarcoma, lymphosarcoma and rhabdomyosarcoma. In some embodiments, the eye cancer is chosen from intraocular melanoma and retinoblastoma. In some embodiments, the hematological cancer is chosen from lymphoma, leukemia such as acute lymphoblastic leukemia (ALL), acute myelogenous leukemia (AML), chronic lymphocytic leukemia (CLL), chronic myelogenous leukemia (CML), hairy cell leukemia, diffuse large B-cell lymphoma (DLBCL), mantle cell lymphoma, non-Hodgkin lymphoma (NHL, including relapsed or refractory NHL), Hodgkin lymphoma, multiple myeloma and combinations of said cancers. In some embodiments, the nervous system cancer is chosen from a cancer of the skull, a cancer of the meninges, brain cancer, glioblastoma, spinal cord cancer, a neuroblastoma, and Lhermitte-Duclos disease.

In another aspect of the present disclosure, a compound, tautomer, deuterated derivative, or pharmaceutically acceptable salt as disclosed herein, including a compound of Formulae I and I′, Compounds 1 to 106, a tautomer thereof, a deuterated derivative of the compound or the tautomer, or a pharmaceutically acceptable salt of the foregoing, or the pharmaceutical composition thereof, is for use in decreasing HPK1 activity. In another aspect, disclosed herein is use of a compound, tautomer, deuterated derivative, or pharmaceutically acceptable salt as disclosed herein, including a compound of Formulae I and I′, Compounds 1 to 106, a tautomer thereof, a deuterated derivative of the compound or the tautomer, or a pharmaceutically acceptable salt of the foregoing, or the pharmaceutical composition thereof, for the manufacture of a medicament for decreasing HPK1 activity. In yet another aspect, disclosed herein is a method of decreasing HPK1 activity, comprising administering a therapeutically effective amount of a compound, tautomer, deuterated derivative, or pharmaceutically acceptable salt as disclosed herein to a subject, including a compound of Formulae I and I′, Compounds 1 to 106, a tautomer thereof, a deuterated derivative of the compound or the tautomer, or a pharmaceutically acceptable salt of the foregoing, or the pharmaceutical composition thereof. In yet another aspect, disclosed herein is a method of decreasing HPK1 activity, comprising contacting said HPK1 with a compound, tautomer, deuterated derivative, or pharmaceutically acceptable salt as disclosed herein to a subject, including a compound of Formulae I and I′, Compounds 1 to 106, a tautomer thereof, a deuterated derivative of the compound or the tautomer, or a pharmaceutically acceptable salt of the foregoing, or the pharmaceutical composition thereof.

A compound of Formulae I and I′, Compounds 1 to 106, a tautomer thereof, a deuterated derivative of the compound or the tautomer, or a pharmaceutically acceptable salt of the foregoing, or the pharmaceutical composition thereof may be administered once daily, twice daily, or three times daily, for example, for the treatment of a disease, a disorder, or a condition mediated by the degradation of HPK1.

In some embodiments, 2 mg to 1500 mg or 5 mg to 1000 mg of a compound of Formulae I and I′, Compounds 1 to 106, a tautomer thereof, a deuterated derivative of the compound or the tautomer, or a pharmaceutically acceptable salt of the foregoing, or the pharmaceutical composition thereof are administered once daily, twice daily, or three times daily.

A compound of Formulae I and I′, Compounds 1 to 106, a tautomer thereof, a deuterated derivative of the compound or the tautomer, or a pharmaceutically acceptable salt of the foregoing, or the pharmaceutical composition thereof may be administered, for example, by oral, parenteral, sublingual, topical, rectal, nasal, buccal, vaginal, transdermal, patch, pump administration or via an implanted reservoir, and the pharmaceutical compositions would be formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time. Other forms of administration contemplated in the present disclosure are as described in International Patent Application Nos. WO 2013/075083, WO 2013/075084, WO 2013/078320, WO 2013/120104, WO 2014/124418, WO 2014/151142, and WO 2015/023915.

Useful dosages or a therapeutically effective amount of a compound or pharmaceutically acceptable salt thereof as disclosed herein can be determined by comparing their in vitro activity and in vivo activity in animal models. Methods for the extrapolation of effective dosages in mice and other animals, to humans are known to the art; for example, see U.S. Pat. No. 4,938,949.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, pharmaceutically acceptable salts, solvates, and deuterated derivatives disclosed herein are based upon the free base form of the reference compound. For example, “1000 mg of at least one compound chosen from compounds of Formulae I and I′ and pharmaceutically acceptable salts thereof” includes 1000 mg of compound of Formulae I and I′ and a concentration of a pharmaceutically acceptable salt of compounds of Formulae I and I′ equivalent to 1000 mg of compounds of Formulae I and I′.

### Non-Limiting Exemplary Embodiments

1. A compound of Formula (I):

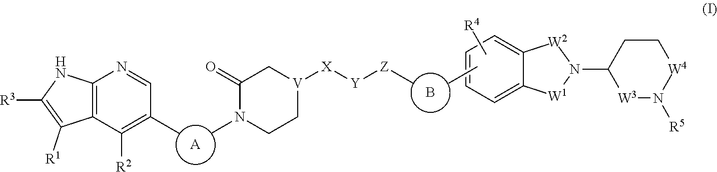

a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) each R², R³ and R⁴ is independently chosen from hydrogen, halogen groups, OR^{x}, SR^{x}, NHR^{x}, N(R^{x})₂, CHR^{x}, and C(R^{x})₂;
(iii) R⁵ is chosen from hydrogen, R^{x}, —CH₂OC(O)R^{x}—, and —CH₂OC(O)C(R^{x}R^{y})NH₂;
(iv) each W¹, W², W³, and W⁴ is independently chosen from C(R^{w})₂ and C(O);
(v) V is chosen from N and CR^{x};
(vi) when V is N, X is absent or is chosen from —C(O)—, —C(O)R^{x}—, —C(S)—, —C(S) R^{x}—, —S(O)₂—, and —S(O)₂R^{x}—; or when V is CR^{x}, X is absent or is chosen from —O—, —S—, —NR^{x}—, —C(O)—, —C(S), and —C(R^{x}R^{y}),
(vii) Y is absent or is chosen from linear, branched, and cyclic alkylene groups and PEG groups;
(viii) Z is absent or is chosen from —O—, —NR^{z}—, —NR^{y}C(O)—, —C(O)—, —C(S)—, and —C(O)O—;
(ix) each R^{w}, R^{x}, R^{y}, and R^{z} is independently chosen from hydrogen, linear, branched, and cyclic alkyl groups, carbocyclic groups, heterocyclic groups, aryl groups, and heteroaryl groups;
(x) ring A is chosen from aryl groups and heteroaryl groups, and
(xi) ring B is absent or is chosen from aryl groups, heteroaryl groups, cycloalkyl groups, and heterocycloalkyl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups.

2. The compound, tautomer, deuterated derivative, or pharmaceutically acceptable salt of embodiment 1, wherein R¹ is chosen from linear, branched, and cyclic alkyl groups; R² is a halogen group; and R³ is chosen from hydrogen, linear, branched, and cyclic alkyl groups.

3. The compound, tautomer, deuterated derivative, or pharmaceutically acceptable salt of embodiment 1 or 2, wherein R¹ is chosen from C₁-C₆ linear, branched, and cyclic alkyl groups.

4. The compound, tautomer, deuterated derivative, or pharmaceutically acceptable salt of embodiment 3, wherein R¹ is chosen from methyl, ethyl, cyclopropyl, and cyclobutyl.

5. The compound, tautomer, deuterated derivative, or pharmaceutically acceptable salt of any of embodiments 1-4, wherein R² is a halogen group.

6. The compound, tautomer, deuterated derivative, or pharmaceutically acceptable salt of embodiment 5, wherein R² is chloro.

7. The compound, tautomer, deuterated derivative, or pharmaceutically acceptable salt of any of embodiments 1-5, wherein R² is hydrogen.

8. The compound, tautomer, deuterated derivative, or pharmaceutically acceptable salt of any of embodiments 1-7, wherein R³ is a halogen group.

9 The compound, tautomer, deuterated derivative, or pharmaceutically acceptable salt of embodiment 8, wherein R³ is chloro.

10. The compound, tautomer, deuterated derivative, or pharmaceutically acceptable salt of any of embodiments 1-7, wherein R³ is hydrogen.

11. The compound, tautomer, deuterated derivative, or pharmaceutically acceptable salt of any of embodiments 1-10, wherein R⁴ is a halogen group.

12. The compound, tautomer, deuterated derivative, or pharmaceutically acceptable salt of any of embodiments 1-11, wherein R⁴ is fluoro.

13. The compound, tautomer, deuterated derivative, or pharmaceutically acceptable salt of any of embodiments 1-12, wherein R⁵ is hydrogen.

14. The compound, tautomer, deuterated derivative, or pharmaceutically acceptable salt of any of embodiments 1-12, wherein R⁵ is chosen from C₁-C₆ linear, branched, and cyclic alkyl groups.

15. The compound, tautomer, deuterated derivative, or pharmaceutically acceptable salt of embodiment 14, wherein R⁵ is chosen from methyl, ethyl, n-propyl, i-propyl, n-butyl, i-butyl, and t-butyl.

16. The compound, tautomer, deuterated derivative, or pharmaceutically acceptable salt of any of embodiments 1-12, wherein R⁵ is —CH₂OC(O)R^{x}—.

17. The compound, tautomer, deuterated derivative, or pharmaceutically acceptable salt of embodiment 16, wherein R^{x} is chosen from methyl, ethyl, n-propyl, i-propyl, n-butyl, i-butyl, and t-butyl.

18. The compound, tautomer, deuterated derivative, or pharmaceutically acceptable salt of any of embodiments 1-12, wherein R⁵ is —CH₂OC(O)C(R^{x}R^{y})NH₂.

19. The compound, tautomer, deuterated derivative, or pharmaceutically acceptable salt of embodiment 18, wherein R^{x} is hydrogen.

20. The compound, tautomer, deuterated derivative, or pharmaceutically acceptable salt of embodiments 18 and 19, wherein R^{y} is chosen from hydrogen, methyl, i-propyl, and benzyl.

21. The compound, tautomer, deuterated derivative, or pharmaceutically acceptable salt of any of embodiments 1-20, wherein X is absent.

22. The compound, tautomer, deuterated derivative, or pharmaceutically acceptable salt of any of embodiments 1-20, wherein X is —C(O)—.

23. The compound, tautomer, deuterated derivative, or pharmaceutically acceptable salt of any of embodiments 1-22, wherein Y is chosen from linear, branched, and cyclic alkylene groups.

24. The compound, tautomer, deuterated derivative, or pharmaceutically acceptable salt of embodiment 23, wherein Y is chosen from C₁-C₁₀ linear alkylene groups.

25. The compound, tautomer, deuterated derivative, or pharmaceutically acceptable salt of any of embodiments 1-22, wherein Y is chosen from PEG groups.

26. The compound, tautomer, deuterated derivative, or pharmaceutically acceptable salt of embodiment 25, wherein Y is chosen from

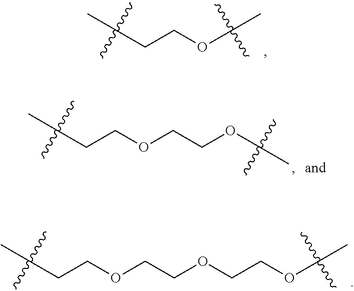 [SMILES: *CCO* ; *CCOCCO* ; *CCOCCOCCO*]

27. The compound, tautomer, deuterated derivative, or pharmaceutically acceptable salt of any of embodiments 1-26, wherein Z is absent.

28. The compound, tautomer, deuterated derivative, or pharmaceutically acceptable salt of any of embodiments 1-26, wherein Z is —C(O)—.

29. The compound, tautomer, deuterated derivative, or pharmaceutically acceptable salt of any of embodiments 1-26, wherein Z is O.

30. The compound, tautomer, deuterated derivative, or pharmaceutically acceptable salt of any of embodiments 1-26, wherein Z is NR^{z}.

31. The compound, tautomer, deuterated derivative, or pharmaceutically acceptable salt of embodiment 30, wherein R^{z} is chosen from hydrogen, linear, branched, and cyclic alkyl groups.

32. The compound, tautomer, deuterated derivative, or pharmaceutically acceptable salt of embodiment 30, wherein R^{z} is hydrogen.

33. The compound, tautomer, deuterated derivative, or pharmaceutically acceptable salt of embodiment 30, wherein R^{z} is methyl.

34. The compound, tautomer, deuterated derivative, or pharmaceutically acceptable salt of any of embodiments 1-33, wherein ring B is absent.

35. The compound, tautomer, deuterated derivative, or pharmaceutically acceptable salt of any of embodiments 1-34, wherein ring B is chosen from optionally substituted heterocycloalkyls.

36. The compound, tautomer, deuterated derivative, or pharmaceutically acceptable salt of embodiment 35, wherein ring B is chosen from

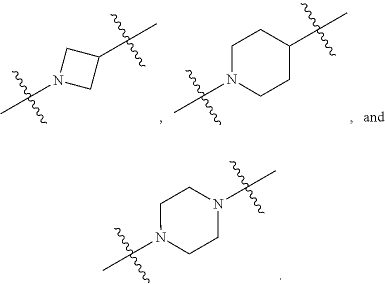 [SMILES: *C1CN(*)C1 ; *C1CCN(*)CC1 ; *N1CCN(*)CC1]

37. The compound, tautomer, deuterated derivative, or pharmaceutically acceptable salt of any of embodiments 1-36, wherein W¹ is C(R^{w})₂.

38. The compound, tautomer, deuterated derivative, or pharmaceutically acceptable salt of embodiment 37, wherein R^{w} is hydrogen.

39. The compound, tautomer, deuterated derivative, or pharmaceutically acceptable salt of any of embodiments 1-36, wherein W¹ is C(O).

40. The compound, tautomer, deuterated derivative, or pharmaceutically acceptable salt of any of embodiments 1-39, wherein W² is C(R^{w})₂.

41. The compound, tautomer, deuterated derivative, or pharmaceutically acceptable salt of embodiment 40, wherein R^{w} is hydrogen.

42. The compound, tautomer, deuterated derivative, or pharmaceutically acceptable salt of any of embodiments 1-39, wherein W² is C(O).

43. The compound, tautomer, deuterated derivative, or pharmaceutically acceptable salt of any of embodiments 1-42, wherein W³ is C(R^{w})₂.

44. The compound, tautomer, deuterated derivative, or pharmaceutically acceptable salt of embodiment 43, wherein R^{w} is hydrogen.

45. The compound, tautomer, deuterated derivative, or pharmaceutically acceptable salt of any of embodiments 1-44, wherein W³ is C(O).

46. The compound, tautomer, deuterated derivative, or pharmaceutically acceptable salt of any of embodiments 1-45, wherein W⁴ is C(R^{w})₂.

47. The compound, tautomer, deuterated derivative, or pharmaceutically acceptable salt of embodiment 46, wherein R^{w} is hydrogen.

48. The compound, tautomer, deuterated derivative, or pharmaceutically acceptable salt of any of embodiments 1-45, wherein W⁴ is C(O).

49. The compound, tautomer, deuterated derivative, or pharmaceutically acceptable salt of any of embodiments 1-45, wherein ring A is a 5 or 6-membered heteroaryl groups.

50. The compound, tautomer, deuterated derivative, or pharmaceutically acceptable salt of embodiment 49, wherein ring A is

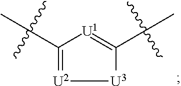

each U¹ and U² is independently chosen from CR^{u} or N; U³ is chosen from O, S, and NR^{u}; and R^{u} is independently chosen from hydrogen, linear, branched, and cyclic alkyl groups, carbocyclic groups, heterocyclic groups, aryl groups, and heteroaryl groups.

51. The compound, tautomer, deuterated derivative, or pharmaceutically acceptable salt of embodiment 50, wherein U¹ is CR^{u}, U² is CR^{u}, and U³ is O.

52. The compound, tautomer, deuterated derivative, or pharmaceutically acceptable salt of embodiment 50, wherein U¹ is CR^{u}, U² is CR^{u}, and U³ is S.

53. The compound, tautomer, deuterated derivative, or pharmaceutically acceptable salt of embodiment 50, wherein U¹ is N, U² is CR^{u}, and U³ is O.

54. The compound, tautomer, deuterated derivative, or pharmaceutically acceptable salt of embodiment 50, wherein U¹ is N, U² is CR^{u}, and U³ is S.

55. The compound, tautomer, deuterated derivative, or pharmaceutically acceptable salt of embodiment 50, wherein U¹ is CR^{u}, U² is N, and U³ is O.

56. The compound, tautomer, deuterated derivative, or pharmaceutically acceptable salt of embodiment 50, wherein U¹ is CR^{u}, U² is N, and U³ is S.

57. The compound, tautomer, deuterated derivative, or pharmaceutically acceptable salt of embodiment 49, wherein ring A is

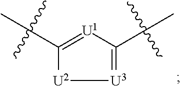

each U¹ and U³ is independently chosen from CR^{u} or N; and U² is chosen from O, S, and NR^{u}; and R^{u} is independently chosen from hydrogen, linear, branched, and cyclic alkyl groups, carbocyclic groups, heterocyclic groups, aryl groups, and heteroaryl groups.

58. The compound, tautomer, deuterated derivative, or pharmaceutically acceptable salt of embodiment 57, wherein U¹ is CR^{u}, U² is O, and U³ is CR^{u}.

59. The compound, tautomer, deuterated derivative, or pharmaceutically acceptable salt of embodiment 57, wherein U¹ is CR^{u}, U² is S, and U³ is CR^{u}.

60. The compound, tautomer, deuterated derivative, or pharmaceutically acceptable salt of embodiment 57, wherein U¹ is N, U² is O, and U³ is CR^{u}.

61. The compound, tautomer, deuterated derivative, or pharmaceutically acceptable salt of embodiment 57, wherein U¹ is N, U² is S, and U³ is CR^{u}.

62. The compound, tautomer, deuterated derivative, or pharmaceutically acceptable salt of embodiment 57, wherein U¹ is CR^{z}, U² is O, and U³ is N.

63. The compound, tautomer, deuterated derivative, or pharmaceutically acceptable salt of embodiment 57, wherein U¹ is CR^{z}, U² is S, and U³ is N.

64. The compound, tautomer, deuterated derivative, or pharmaceutically acceptable salt of embodiment 49, wherein ring A is

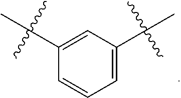 [SMILES: *c1cccc(*)c1]

65. The compound, tautomer, deuterated derivative, or pharmaceutically acceptable salt of embodiment 49, wherein ring A is

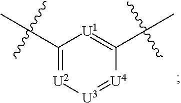

each U¹, U², U³, and U⁴ is independently chosen from CR^{z} or N; and R^{u} is independently chosen from hydrogen, linear, branched, and cyclic alkyl groups, carbocyclic groups, heterocyclic groups, aryl groups, and heteroaryl groups 66. The compound, tautomer, deuterated derivative, or pharmaceutically acceptable salt of embodiment 65, wherein U¹ is N, U² is CR^{u}, U³ is CR^{u}, and U⁴ is CR^{u}.

67. The compound, tautomer, deuterated derivative, or pharmaceutically acceptable salt of embodiment 65, wherein U¹ is CR^{u}, U² is N, U³ is CR^{u}, and U⁴ is CR^{u}.

68. The compound, tautomer, deuterated derivative, or pharmaceutically acceptable salt of embodiment 65, wherein U¹ is CR^{u}, U² is CR^{u}, U³ is N, and U⁴ is CR^{u}.

69. The compound, tautomer, deuterated derivative, or pharmaceutically acceptable salt of embodiment 65, wherein U¹ is CR^{u}, U² is CR^{u}, U³ is CR^{u}, and U⁴ is N.

70. The compound, tautomer, deuterated derivative, or pharmaceutically acceptable salt of embodiment 65, wherein U¹ is N, U² is N, U³ is CR^{u}, and U⁴ is CR^{u}.

71. The compound, tautomer, deuterated derivative, or pharmaceutically acceptable salt of embodiment 65, wherein U¹ is N, U² is CR^{u}, U³ is N, and U⁴ is CR^{u}.

72. The compound, tautomer, deuterated derivative, or pharmaceutically acceptable salt of embodiment 65, wherein U¹ is CR^{u}, U² is CR^{u}, U³ is CR^{u}, and U⁴ is N.

73. The compound, tautomer, deuterated derivative, or pharmaceutically acceptable salt of embodiment 65, wherein U¹ is N, U² is CR^{u}, U³ is CR^{u}, and U⁴ is N.

74. A compound chosen from the compounds of Table 1, a tautomer thereof, a deuterated derivative of the compound or the tautomer, or a pharmaceutically acceptable salt of the foregoing.

75. A pharmaceutical composition comprising a compound, tautomer, deuterated derivative, or pharmaceutically acceptable salt according to any one of embodiments 1-74 and at least one pharmaceutically acceptable carrier.

76. A method for treating or alleviating a disease, a disorder or a condition mediated by the degradation of hematopoietic progenitor kinase 1 (HPK1), comprising administering to a subject in need thereof a therapeutically effective amount of a compound, tautomer, deuterated derivative, or pharmaceutically acceptable salt according to any one of the embodiments 1-74 or the pharmaceutical composition according to embodiment 75.

77. A method for decreasing HPK1 activity in a disease, a disorder or a condition, comprising administering to a subject in need thereof a therapeutically effective amount of a compound, tautomer, deuterated derivative, or pharmaceutically acceptable salt according to any one of the embodiments 1-74 or the pharmaceutical composition according to embodiment 75.

78. The method of embodiment 145, wherein the disease, the disorder, or the condition is chosen from an HPK1-related disease.

79. The method of embodiment 145 wherein the HPK1-related disease is chosen from cancer, a dysregulated immune response, or a disease involved in aberrant HPK1 expression, activity, and/or signaling.

80. The method of embodiment 79, wherein the cancer is chosen from brain cancer, breast cancer, respiratory tract and/or lung cancer, a reproductive organ cancer, bone cancer, digestive tract cancer, urinary tract cancer, eye cancer, liver cancer, kidney cancer, skin cancer, head and neck cancer, anal cancer, nervous system cancer, thyroid cancer, parathyroid cancer, a lymphoma, a sarcoma, and a leukemia.

81. The method of embodiment 80, wherein the brain cancer is chosen from brain stem and hypothalamic glioma, cerebellar and cerebral astrocytoma, glioblastoma multiforme, medulloblastoma, ependymoma, neuroectodermal, and pineal tumor.

82. The method of embodiment 80, wherein the liver cancer is chosen from hepatocellular carcinoma (liver cell carcinomas with or without fibrolamellar variant), cholangiocarcinoma (intrahepatic bile duct carcinoma) and mixed hepatocellular cholangiocarcinoma.

83. The method of embodiment 80, wherein the respiratory tract and/or lung cancer is chosen from small cell lung cancer, non-small cell lung cancer, bronchial adenoma, and pleuropulmonary blastoma.

84. The method of embodiment 80, wherein the digestive tract cancer is chosen from anal, colon, rectal, gallbladder, gastric, esophagus cancer, stomach, pancreas, salivary gland, small-intestine, and colorectal cancer.

85 The method of embodiment 80, wherein the kidney cancer is chosen from renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor.

86. The method of embodiment 80, wherein the skin cancer is chosen from malignant melanoma, squamous cell carcinoma, Kaposi's sarcoma, Merkel cell skin cancer and non-melanoma skin cancer.

87. The method of embodiment 80, wherein the head and neck cancer is chosen from squamous cell cancer of the head and neck, laryngeal, hypopharyngeal, nasopharyngeal, oropharyngeal cancer, nasal and paranasal cancers, salivary gland cancer, lip and oral cavity cancer and squamous cell.

88. The method of embodiment 80, wherein the reproductive organ cancer is chosen from prostate cancer, testicular cancer, endometrial cancer, cervical cancer, ovarian cancer, vaginal cancer, vulvar cancer, and uterus sarcoma.

89. The method of embodiment 88, wherein the ovarian cancer is chosen from serous tumor, endometrioid tumor, mucinous cystadenocarcinoma, granulasa cell tumor, Sertoli-Leydig cell tumor, and arrhenoblastoma.

90. The method of embodiment 88, wherein the cervical cancer is chosen from squamous cell carcinoma, adenocarcinoma, adenosquamous carcinoma, small cell carcinoma, neuroendocrine tumor, glassy cell carcinoma, and villogladular adenocarcinoma.

91. The method of embodiment 80, wherein the bone cancer is chosen from osteogenic sarcoma, fibrosarcoma, malignant fibrous histiocytoma, chondrosarcoma, Ewing's sarcoma, malignant lymphoma, multiple myeloma, malignant giant cell tumor chordoma, osteochondroma, benign chondroma, chondroblastoma, chondromyxofibroma, osteoid osteoma, and giant cell tumors.

92. The method of embodiment 80, wherein the breast cancer is chosen from triple negative breast cancer, invasive ductal carcinoma, invasive lobular carcinoma, ductal carcinoma in situ, and lobular carcinoma in situ.

93. The method of embodiment 80, wherein the sarcoma is chosen from sarcoma of the soft tissue, chondrosarcoma, Ewing's sarcoma, angiosarcoma, fibrosarcoma, myxoma, rhabdomyoma, fibroma, lipoma, harmatoma, teratoma, osteosarcoma, malignant fibrous histiocytoma, liposarcoma, lymphosarcoma and rhabdomyosarcoma.

94. The method of embodiment 80, wherein the eye cancer is chosen from intraocular melanoma and retinoblastoma.

95. The method of embodiment 80, wherein the hematological cancer is chosen from lymphoma, leukemia such as acute lymphoblastic leukemia (ALL), acute myelogenous leukemia (AML), chronic lymphocytic leukemia (CLL), chronic myelogenous leukemia (CML), hairy cell leukemia, diffuse large B-cell lymphoma (DLBCL), mantle cell lymphoma, non-Hodgkin lymphoma (NHL, including relapsed or refractory NHL), Hodgkin lymphoma, multiple myeloma and combinations of said cancers.

96. The method of embodiment 80, wherein the nervous system cancer is chosen from a cancer of the skull, a cancer of the meninges, brain cancer, glioblastoma, spinal cord cancer, a neuroblastoma, and Lhermitte-Duclos disease.

97. The method of embodiment 76, further comprising the administration to the subject an existing standard treatment or an FDA-approved therapy.

### EXAMPLES

### Synthesis of Compounds

To fully understand the present disclosure, the following examples are disclosed. It should be understood that these examples are for illustrative purposes only and are not to be construed as limiting the present disclosure in any manner.

All the specific and generic compounds, and the intermediates disclosed for making those compounds, are considered to be part of the present disclosure.

The compounds of the present disclosure may be made according to standard chemical practices or as disclosed herein. Throughout the following synthetic schemes and in the descriptions for preparing compounds of Formulae I and I′, Compounds 1 to 106, pharmaceutically acceptable salts of any of those compounds, solvates of any of the foregoing, and deuterated derivatives of any of the foregoing, the following abbreviations are used:

### Abbreviations

ACN=acetonitrile
Boc₂O=di-tert-butyl dicarbonate
DCE=1,2-dichloroethane
DCM=dichloromethane
DIEA=N,N-Diisopropylethylamine or N-ethyl-N-isopropyl-propan-2-amine
DMA=N,N-dimethylformamide
DMAP=dimethylamino pyridine
DME=dimethoxyethane
DMF=dimethylformamide
DMSO=dimethyl sulfoxide
EtOAc/EA=Ethyl Acetate
EtOH=ethanol
HOAc=acetic acid
KOAc=potassium acetate
MeOH=methanol
NaOAc=sodium acetate
NMP=N-methyl-2-pyrrolidone
PE=petroleum ether
Pd(dppf)₂Cl₂=[1,1′-Bis(diphenylphosphino) ferrocene]dichloropalladium (II)
rt=room (ambient) temperature
T₃P=2,4,6-Tripropyl-1,3,5,2,4,6-trioxatriphosphorinane-2,4,6-trioxide
TBA=tertiary butyl alcohol
TEA=triethylamine
TFA=trifluoroacetic acid
TFAA=trifluoroacetic anhydride
THF=tetrahydrofuran
TLC=thin layer chromatography
TsCl=p-toluene sulfonyl chloride

### General Preparations

### Synthesis of Intermediates

### Intermediate A: 1-(3-(4-chloro-3-ethyl-1H-pyrrolo[2,3-b]pyridin-5-yl)phenyl)piperazin-2-one

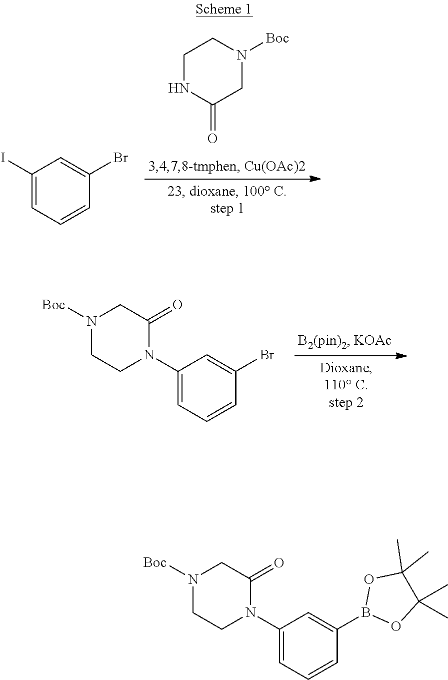 [SMILES: CC(C)(C)OC(=O)N1CCNC(=O)C1 ; Brc1cccc(I)c1 ; CC(C)(C)OC(=O)N1CCN(c2cccc(Br)c2)C(=O)C1 ; CC(C)(C)OC(=O)N1CCN(c2cccc(B3OC(C)(C)C(C)(C)O3)c2)C(=O)C1]

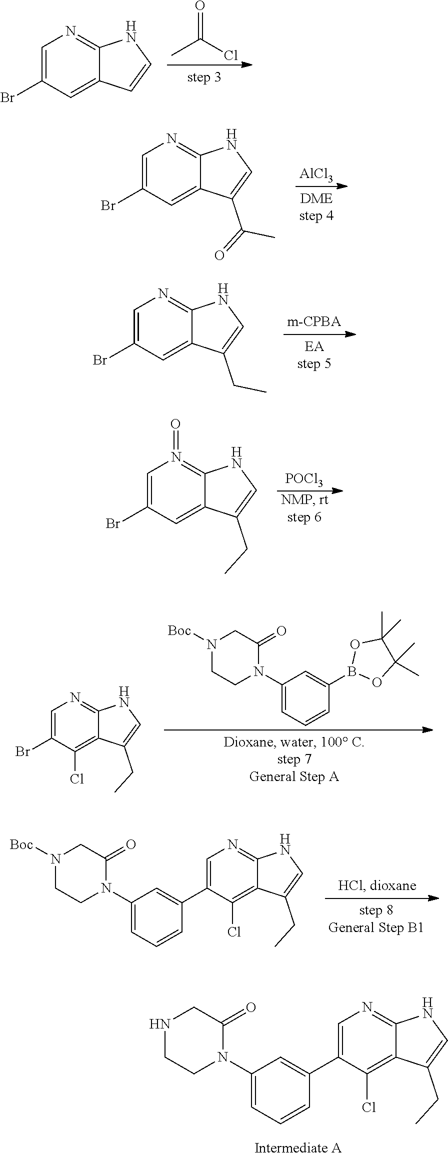

Step 1. Preparation of tert-butyl 4-(3-bromophenyl)-3-oxopiperazine-1-carboxylate: To a solution of 1-bromo-3-iodobenzene (77.33 g, 0.273 mol), tert-butyl 3-oxopiperazine-1-carboxylate (50 g, 0.248 mol), 3,4,7,8-tetramethyl-1,10-phenanthroline (17.62 g, 0.074 mol) in dioxane (1000 mL) under N₂ was added Cu(OAc)₂ (9 g, 0.05 mol) and Cs₂CO₃ (162 g, 0.5 mol). The reaction mixture was stirred at 100° C. for 16 hrs. The reaction was filtered and concentrated. The residue was slurry with EA/PE (1:10, 550 mL) for 2 hrs. The solid was filtered to afford product (70 g, 79% yield) as a yellow solid. Mass (m/z): 376.7 [M+Na]⁺.

Step 2. Preparation of tert-butyl 3-oxo-4-(3-(4,4,5,5-tetramethyl-1,3,2-dioxaborolan-2-yl)phenyl)piperazine-1-carboxylate: To a solution of tert-butyl 4-(3-bromophenyl)-3-oxopiperazine-1-carboxylate (107 g, 0.3 mol), 4,4,5,5-tetramethyl-2-(4,4,5,5-tetramethyl-1,3,2-dioxaborolan-2-yl)-1,3,2-dioxaborolane (91.5 g, 0.36 mol) and KOAc (88.45 g, 0.9 mol) in dioxane (1500 mL) under N₂ was added Pd(dppf)₂Cl₂ (14.9 g, 0.018 mol). The reaction mixture was stirred at 110° C. for 16 hrs. The reaction was filtered and concentrated. The reaction was added water (500 mL), extracted with EA (500 mL×3). The combined organic layers were washed with brine (1000 mL), dried over sodium sulfate, filtered and concentrated. The residue was purified by combi-flash with EA/PE (1:2) to afford product (92 g, 75.9% yield) as a white solid. Mass (m/z): 402.6 [M+H]⁺.

Step 3. Preparation of 1-(5-bromo-1H-pyrrolo[2,3-b]pyridin-3-yl) ethan-1-one: To a solution of 5-bromo-1H-pyrrolo[2,3-b]pyridine (50 g, 0.25 mol) in DCM (550 mL) was added AlCl₃ (101.27 g, 0.76 mol) and acetyl chloride (21.92 g, 0.28 mol) at 0° C. under N₂. The reaction mixture was stirred at rt under N₂ for 7 hrs. MeOH (300 mL) was added to the reaction mixture and the solvent was removed under reduced pressure. The reaction solution was adjusted to pH 6-7 with 3 N aqueous NaOH and extracted with EA (500 mL×3). The combined organic layer was washed with brine (300 mL×3), then dried over with anhydrous Na₂SO₄. After filtration, the solution was concentrated under vacuum, and the crude product was purified by Combiflash (PE/EtOAc=2:1) to give the product 1-(5-bromo-1H-pyrrolo[2,3-b]pyridin-3-yl) ethan-1-one as yellow solid (43.24 g, 71%). Mass (m/z): 241.0 [M+H]⁺.

Step 4. Preparation of 5-bromo-3-ethyl-1H-pyrrolo[2,3-b]pyridine: To a solution of AlCl₃ (27.8 g, 0.20 mol) in DME (200 mL) was added LiAlH₄ (4.39 g, 0.1 mol) and 1-(5-bromo-1H-pyrrolo[2,3-b]pyridin-3-yl) ethan-1-one (10 g, 0.04 mol) at 0° C. . . . The reaction mixture was stirred at rt under N₂ for 3 hs. After the reaction completed, H₂O (500 mL) was added to the reaction mixture, and then extracted with EA (200 mL×3). The combined organic layer was washed with brine (100 mL×2), then dried over with anhydrous Na₂SO₄. The reaction mixture was filtered, the filtrate was concentrated under vacuum to afford compound product 5-bromo-3-ethyl-1H-pyrrolo[2,3-b]pyridine as yellow solid (11.5 g, 74%). Mass (m/z): 225.0 [M+H]⁺.

Step 5. Preparation of 5-bromo-3-ethyl-1H-pyrrolo[2,3-b]pyridine 7-oxide: To a solution of 5-bromo-3-ethyl-1H-pyrrolo[2,3-b]pyridine (25 g, 0.11 mol) in EA (100 mL) was added 3-Chloroperoxybenzoic acid (26.84 g, 0.155 mol). The reaction mixture was stirred at RT for 3 hrs. The solution was washed with sat. Na₂CO₃ (20 mL) and brine (20 mL), then dried over with anhydrous Na₂SO₄. The reaction mixture was filtered, the filtrate was concentrated to dryness to give the desired product as a white solid (17.4 g, yield: 64.6%). Mass (m/z): 240.7 [M+H]⁺.

Step 6. Preparation of 5-bromo-4-chloro-3-ethyl-1H-pyrrolo[2,3-b]pyridine: To a solution of 5-bromo-3-ethyl-1H-pyrrolo[2,3-b]pyridine 7-oxide (17.3 g, 71.8 mmol) in NMP (15 mL) was added phosphoryl trichloride (55.05 g, 35.9 mmol) at 0° C. The reaction mixture was stirred at rt for 16 hrs. The mixture was quenched with water (50 mL), extracted with EA (30 mL×3), washed with sat brine, filtrated, concentrated, the residue was purified by flash column (PE/EA=5:1) to give the desired product as a white solid (4.1 g, yield: 22%). Mass (m/z): 258.7 [M+H]⁺.

Step 7. (General Step A) Preparation of tert-butyl 4-(3-(4-chloro-3-ethyl-1H-pyrrolo[2,3-b]pyridin-5-yl)phenyl)-3-oxopiperazine-1-carboxylate: To a mixture of 5-bromo-4-chloro-3-ethyl-1H-pyrrolo[2,3-b]pyridine (4.1 g, 15.8 mmol), tert-butyl 3-oxo-4-(3-(4,4,5,5-tetramethyl-1,3,2-dioxaborolan-2-yl)phenyl)piperazine-1-carboxylate (7.01 g, 17.38 mmol) and K₂CO₃ (6.55 g, 4.74 mmol) in dioxane/H₂O (10:1, 50 mL) under N₂ was added Pd(dppf)Cl₂ (1.16 g, 1.58 mmol). The reaction mixture was stirred at 90° C. for 4 hrs. The reaction was filtered and concentrated. The residue was purified by combi-flash with DCM/PE (1:2) to afford product (5.8 g, yield: 80%) as a yellow solid. Mass (m/z): 455.2 [M+H]⁺.

Step 8. (General Step B1) Preparation of 1-(3-(4-chloro-3-ethyl-1H-pyrrolo[2,3-b]pyridin-5-yl)phenyl)piperazin-2-one: To a mixture of tert-butyl 4-(3-(4-chloro-3-ethyl-1H-pyrrolo[2,3-b]pyridin-5-yl)phenyl)-3-oxopiperazine-1-carboxylate (5.8 g, 12.7 mmol) in DCM (20 mL) was added HCl in dioxane (20 mL). The reaction mixture was stirred at rt for 2 hrs. The reaction mixture was concentrated under reduced pressure. The residue was slurry with DCM (10 mL) for 1 h. The solid was filtered to afford Intermediate A (4.1 g, yield: 91%) HCl salt as a yellow solid. Mass (m/z): 354.7 [M+H]⁺. ¹H NMR (400 MHZ, DMSO) δ 12.02 (s, 1H), 10.25 (s, 2H), 8.17 (s, 1H), 7.55 (d, J=7.8 Hz, 1H), 7.44 (dd, J=19.0, 10.0 Hz, 4H), 4.00 (t, J=5.0 Hz, 2H), 3.87 (s, 2H), 3.55 (s, 2H), 2.97-2.90 (m, 2H), 1.28 (t, J=7.4 Hz, 3H).

### Intermediate B: 1-(3-(2-chloro-3-ethyl-1H-pyrrolo[2,3-b]pyridin-5-yl)phenyl)piperazin-2-one

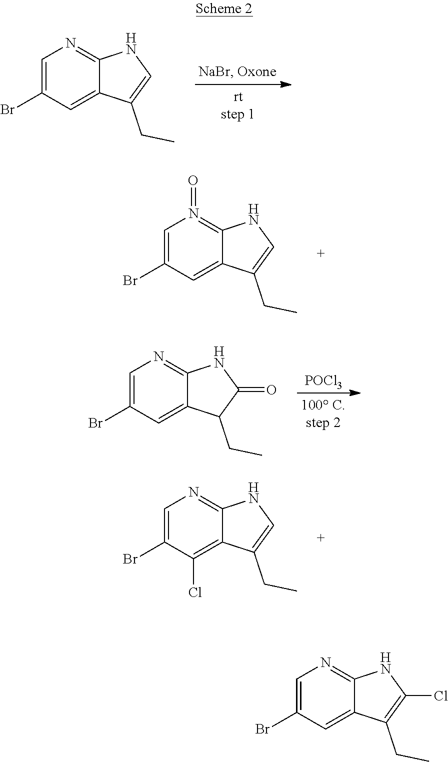

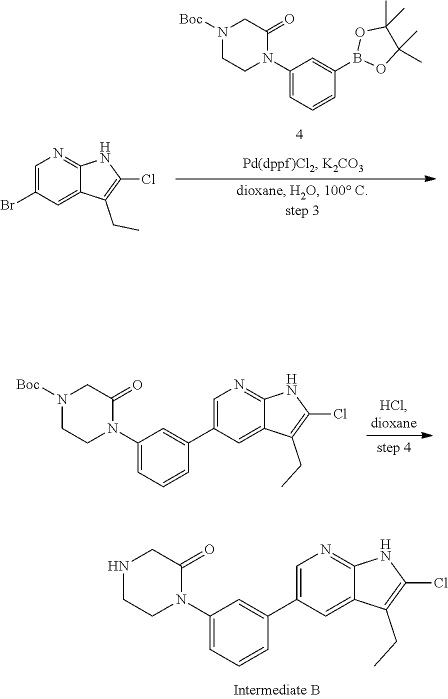 [SMILES: CC(C)(C)OC(=O)N1CCN(c2cccc(B3OC(C)(C)C(C)(C)O3)c2)C(=O)C1 ; CCc1c(Cl)[nH]c2ncc(Br)cc12 ; CCc1c(Cl)[nH]c2ncc(-c3cccc(N4CCN(C(=O)OC(C)(C)C)CC4=O)c3)cc12 ; CCc1c(Cl)[nH]c2ncc(-c3cccc(N4CCNCC4=O)c3)cc12]

Step 1. Preparation of 5-bromo-3-ethyl-1H-pyrrolo[2,3-b]pyridine 7-oxide: To a solution of 5-bromo-3-ethyl-1H-pyrrolo[2,3-b]pyridine (27 g, 120 mmol) in TBA/H₂O=20/1 (63 mL) was added NaBr (0.37 g, 3.6 mmol) and Oxone (55.35 g, 90 mmol). The reaction mixture was stirred at rt under N₂ for 3 hrs. After the reaction completed, H₂O (500 mL) was added to the reaction mixture, and then extracted with EA (500 mL×3). The combined organic layer was washed with brine (300 mL×3), then dried over with anhydrous Na₂SO₄. After filtration, the solution was concentration under vacuum, and the crude product was purified by Combiflash (DCM/MeOH-0˜30%) to give the product 5-bromo-3-ethyl-1H-pyrrolo[2,3-b]pyridine 7-oxide as brown solid (3.5 g, 12%). Mass (m/z): 240.9 [M+H]⁺ and product 5-bromo-3-ethyl-1,3-dihydro-2H-pyrrolo[2,3-b]pyridin-2-one as white solid (8.9 g, 30%). Mass (m/z): 240.9 [M+H]⁺.

Step 2. Preparation of 5-bromo-4-chloro-3-ethyl-1H-pyrrolo[2,3-b]pyridine: To a solution of 5-bromo-3-ethyl-1H-pyrrolo[2,3-b]pyridine 7-oxide (3.5 g, 14.5 mmol) in POCl₃ (30 mL). The reaction mixture was stirred at 90° C. under N₂ for 16 hrs. After the reaction completed, H₂O (200 mL) was added to the reaction mixture, and then extracted with EA (100 mL×3). The combined organic layer was washed with brine (100 mL×2), then dried over with anhydrous Na₂SO₄. After filtration, the solution was concentration under vacuum, and the crude product was purified by Combiflash (PE/EA=0˜50%) to give the by-product as yellow solid (1.24 g, 33%). Mass (m/z): 260.9 [M+H]⁺ and desired product 5-bromo-4-chloro-3-ethyl-1H-pyrrolo[2,3-b]pyridine as white solid (1.63 g, 43%). Mass (m/z): 260.9 [M+H]⁺.

Step 3. Preparation of tert-butyl 4-(3-(2-chloro-3-ethyl-1H-pyrrolo[2,3-b]pyridin-5-yl)phenyl)-3-oxopiperazine-1-carboxylate: Following General Step A, tert-butyl 4-(3-(2-chloro-3-ethyl-1H-pyrrolo[2,3-b]pyridin-5-yl)phenyl)-3-oxopiperazine-1-carboxylate was prepared as a yellow solid (1.5 g, 84.7% yield). Mass (m/z): 455.2 [M+H]⁺.

Step 4. Preparation of 1-(3-(2-chloro-3-ethyl-1H-pyrrolo[2,3-b]pyridin-5-yl)phenyl) piperazin-2-one (Intermediate B): Following General Step B1, product was prepared as a yellow solid (1.2 g, 92% yield). Mass (m/z): 355.1 [M+H]⁺. ¹HNMR (400 MHZ, CD₃OD) δ 8.76 (s, 1H), 8.62 (s, 1H), 7.80-7.72 (m, 2H), 7.64 (t, J=8.0 Hz, 1H), 7.45 (d, J=8.0 Hz, 1H), 4.08-4.04 (m, 4H), 3.77-3.68 (m, 2H), 2.88 (q, J=7.6 Hz, 2H), 1.29 (t, J=7.6 Hz, 3H).

### Intermediate C: 1-(3-(4-chloro-3-cyclopropyl-1H-pyrrolo[2,3-b]pyridin-5-yl)phenyl)piperazin-2-one

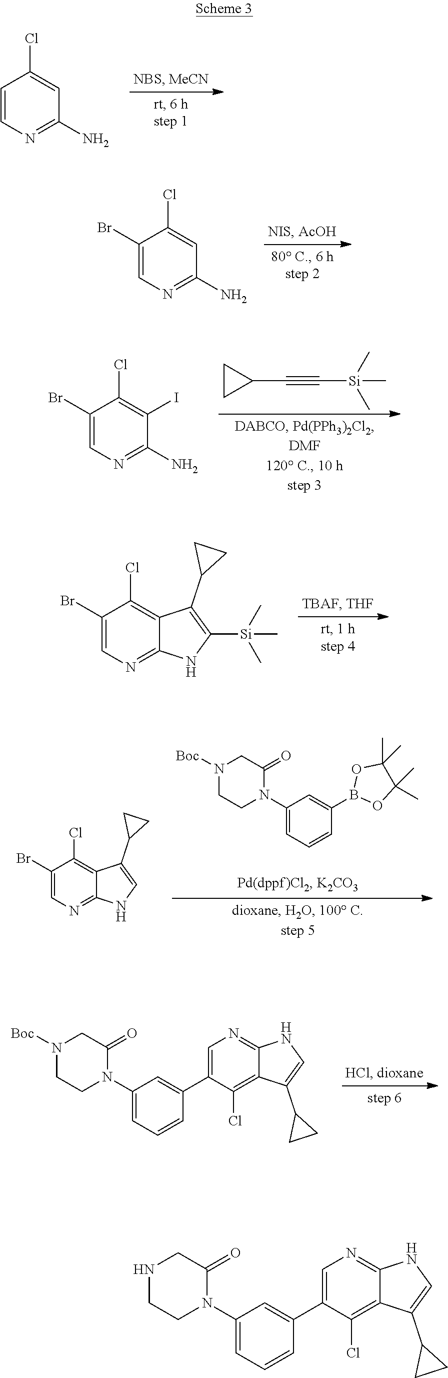

Step 1. Preparation of 5-bromo-4-chloropyridin-2-amine: To a solution of compound 4-chloropyridin-2-amine (300 g, 2.34 mol, 1.0 eq) in acetonitrile (3000 mL) was added NBS (458 g, 2.57 mol, 1.1 eq) in several portions. The reaction mixture was stirred at room temperature for 6 hrs. Then the reaction was poured into water, filtered. The filter cake was washed with PE and dried to afford compound 5-bromo-4-chloropyridin-2-amine (407 g, 83.9% yield) as a yellow solid. Mass (m/z): 207 [M+H]⁺. ¹HNMR (400 MHZ, DMSO-d₆) δ 8.10 (s, 1H), 6.67 (s, 1H), 6.45 (s, 2H).

Step 2. Preparation of 5-bromo-4-chloro-3-iodopyridin-2-amine: To a solution of compound 5-bromo-4-chloropyridin-2-amine (407 g, 1.97 mol, 1.0 eq) in AcOH (2000 mL) was added NIS (666 g, 2.96 mol, 1.5 eq) in several portions. The reaction mixture was stirred at 80° C. for 4 hrs. The reaction was cooled to room temperature, poured into ice water (5000 mL), adjusted PH >7 with K₂CO₃, extracted with EA (5000 mL×3), washed with a solution of Na₂SO₃ (5000 mL) and brine (5000 mL). The organic phase was concentrated in vacuo to afford compound 5-bromo-4-chloro-3-iodopyridin-2-amine (500 g, 76.3% yield) as a yellow solid. Mass (m/z): 332.7 [M+H]⁺. ¹HNMR (400 MHZ, DMSO-d₆) δ 8.10 (s, 1H), 6.62 (s, 2H).

Step 3. Preparation of 5-bromo-4-chloro-3-cyclopropyl-2-(trimethylsilyl)-1H-pyrrolo[2,3-b]pyridine: To a solution of compound 5-bromo-4-chloro-3-iodopyridin-2-amine (100 g, 0.300 mol, 1.0 eq), DABCO (101 g, 0.900 mol, 3.0 eq) in DMF (2000 mL) under N₂ was added Pd(PPh₃)₂Cl₂ (21.1 g, 0.03 mol, 0.1 eq). Then compound (cyclopropylethynyl)trimethylsilane (166 g, 1.20 mol, 4.0 eq) was added. The reaction was degassed for 3 times under N₂. The reaction mixture was stirred at 120° C. for 10 hrs. The reaction was filtered, quenched with water (2000 mL), extracted with EA (2000 mL×3), washed with brine (2000 mL), dried over Na₂SO₄, filtered, concentrated in vacuo. The crude was purified by chromatography on sili-gel with THF/PE (1:15) to afford compound 5-bromo-4-chloro-3-cyclopropyl-2-(trimethylsilyl)-1H-pyrrolo[2,3-b]pyridine (27 g, 26.2% yield) as a yellow solid. Mass (m/z): 344.9 [M+H]⁺.

Step 4. Preparation of 5-bromo-4-chloro-3-cyclopropyl-1H-pyrrolo[2,3-b]pyridine: To a mixture of compound 5-bromo-4-chloro-3-cyclopropyl-2-(trimethylsilyl)-1H-pyrrolo[2,3-b]pyridine (27 g, 79.0 mmol, 1.0 eq) in THF (237 mL) was added TBAF in THF (1.0 M, 237 mL, 3.0 eq) and H₂O (4.27 g, 237 mmol, 3.0 eq). The reaction mixture was stirred at room temperature for 1 hrs. The reaction was quenched with water (1000 mL), extracted with EA (1000 mL×3), washed with brine (1000 mL), dried over Na₂SO₄, filtered, concentrated in vacuo. The crude was purified by chromatography on sili-gel with THF/PE (1:4) to afford the product compound 5-bromo-4-chloro-3-cyclopropyl-1H-pyrrolo[2,3-b]pyridine (15 g, 70.4% yield) as a faint yellow solid. Mass (m/z): 272.9 [M+H]⁺. ¹HNMR (400 MHZ, DMSO-d₆) δ 11.92 (s, 1H), 8.36 (s, 1H), 7.33-7.34 (d, J=4.0 Hz, 1H), 2.11-2.16 (m, 1H), 0.84-0.86 (m, 2H), 0.62-0.64 (m, 2H).

Step 5. Preparation of tert-butyl 4-(3-(4-chloro-3-cyclopropyl-1H-pyrrolo[2,3-b]pyridin-5-yl)phenyl)-3-oxopiperazine-1-carboxylate: Following General Step A, product tert-butyl 4-(3-(4-chloro-3-cyclopropyl-1H-pyrrolo[2,3-b]pyridin-5-yl)phenyl)-3-oxopiperazine-1-carboxylate was obtained (250 mg, 21%) as a black oil. MS: m/z=466.9 (M+1, ESI+).

Step 6. Preparation of 1-(3-(4-chloro-3-cyclopropyl-1H-pyrrolo[2,3-b]pyridin-5-yl)phenyl)piperazin-2-one: Following General Step B1, product 1-(3-(4-chloro-3-cyclopropyl-1H-pyrrolo[2,3-b]pyridin-5-yl)phenyl)piperazin-2-one was obtained (14 mg, 6.4% yield) as a yellow solid. MS: m/z=367 (M+1, ESI+).

### Intermediate D: 1-(3-(4-chloro-3-(2,2-difluoroethyl)-1H-pyrrolo[2,3-b]pyridin-5-yl)phenyl)piperazin-2-one

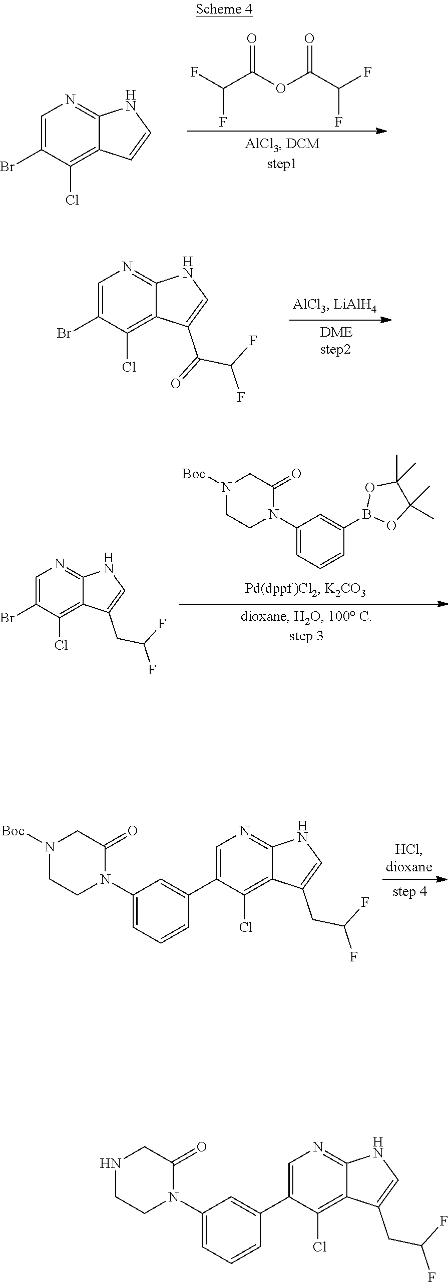

Step 1. Preparation of 1-{5-bromo-4-chloro-1H-pyrrolo[2,3-b]pyridin-3-yl}-2,2-difluoroethanone: To a solution of 3-bromo-4-chloro-7H-pyrrolo[2,3-b]pyridine (500 mg, 2.16 mol) in DCM (10 mL) was added AlCl₃ (863.78 mg, 6.48 mmol) and 2,2-difluoroacetyl 2,2-difluoroacetate (751.9 mg, 4.32 mol) at 0° C. The reaction mixture was stirred at 25° C. under N₂ for 7 hrs. MeOH (30 mL) was added to the reaction mixture and the solvent was removed under reduced pressure. The residue was adjusted to pH 6-7 with 3 N aqueous NaOH and extracted with EA (100 mL×3). The combined organic layers were washed with brine (30 mL×3), then dried over Na₂SO₄. After filtration, the filtrate was concentrated under vacuum, the residue was purified by Combiflash (eluting with PE/EtOAc-2:1) to give the product as yellow solid (200 mg, 11.67%). Mass (m/z): 308.7 [M+H]⁺.

Step 2. Preparation of 3-bromo-4-chloro-3-(2,2-difluoroethyl)-1H-pyrrolo[2,3-b]pyridine: To a solution of AlCl₃ (200 mg, 0.65 mmol) in DME (10 mL) was added LiAlH₄ (64.62 mg, 1.62 mmol) and 1-{5-bromo-4-chloro-1H-pyrrolo[2,3-b]pyridin-3-yl}-2,2-difluoroethanone (430.69 g, 3.23 mmol) at 0° C. . . . The reaction mixture was stirred at 25° C. under N₂ for 3 hrs. After the reaction completed, H₂O (100 mL) was added, then extracted with EA (20 mL×3). The combined organic layer was washed with brine (10 mL×2), then dried over Na₂SO₄. The reaction mixture was filtered, the filtrate was concentrated under vacuum and purified by combi-flash, eluting with PE/EA (1:1) to afford compound product 3-bromo-4-chloro-3-(2,2-difluoroethyl)-1H-pyrrolo[2,3-b]pyridine (50 mg, 13.09%) as a brown solid compound. Mass (m/z): 295.0 [M+H]⁺.

Step 3. Preparation of tert-butyl 4-(3-(4-chloro-3-(2,2-difluoroethyl)-1H-pyrrolo[2,3-b]pyridin-5-yl)phenyl)-3-oxopiperazine-1-carboxylate: Following General Step A, product tert-butyl 4-(3-(4-chloro-3-(2,2-difluoroethyl)-1H-pyrrolo[2,3-b]pyridin-5-yl)phenyl)-3-oxopiperazine-1-carboxylate was obtained as a brown solid (70 mg, yield: 33%). Mass (m/z): 491.1 [M+H]⁺.

Step 4. Preparation of 1-(3-(4-chloro-3-(2,2-difluoroethyl)-1H-pyrrolo[2,3-b]pyridin-5-yl)phenyl)piperazin-2-one: Following General Step B1, product 1-(3-(4-chloro-3-(2,2-difluoroethyl)-1H-pyrrolo[2,3-b]pyridin-5-yl)phenyl)piperazin-2-one was obtained as a yellow solid (5 mg, 8%). Mass (m/z): 391.0 [M+H]⁺.

### Intermediate E: 1-(6-(4-chloro-3-cyclopropyl-1H-pyrrolo[2,3-b]pyridin-5-yl)pyridin-2-yl)piperazin-2-one

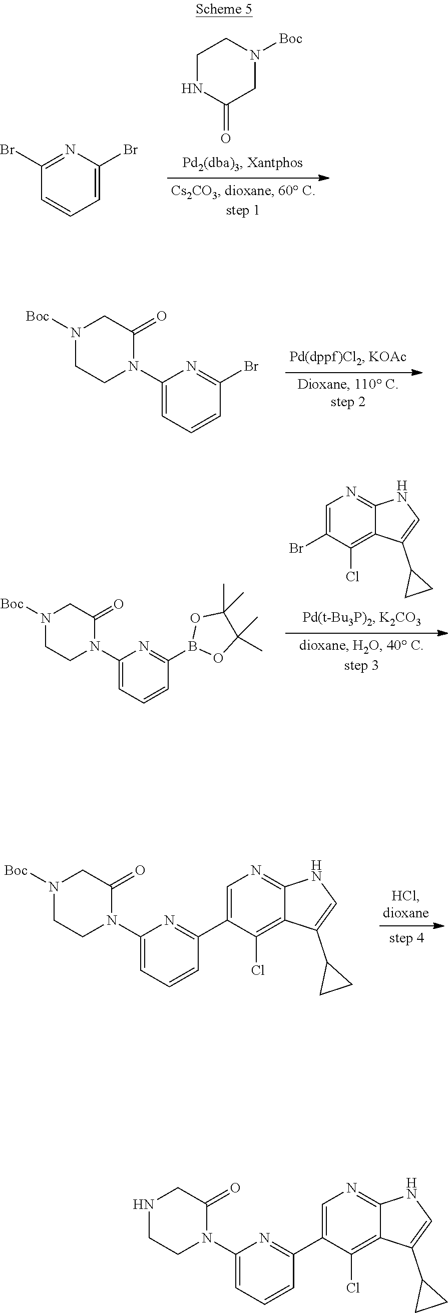

Step 1. Preparation of tert-butyl 4-(6-bromopyridin-2-yl)-3-oxopiperazine-1-carboxylate: To a solution of 2,6-dibromopyridine (117.5 g, 0.50 mol, 2.0 eq), tert-butyl 3-oxopiperazine-1-carboxylate (50.0 g, 0.25 mol, 1.0 eq), Cs₂CO₃ (163 g, 0.50 mol, 2.0 eq) in dioxane (1000 mL) under N₂ was added Pd₂(dba)₃ (11.5 g, 0.0125 mol, 0.05 eq) and Xantphos (14.5 g, 0.025 mol, 0.1 eq). The reaction mixture was stirred at 60° C. for 2 hrs. The reaction was filtered and concentrated. The reaction was added water (500 mL), extracted with EA (500 mL×3). The combined organic layers were washed with brine (1000 mL), dried over sodium sulfate, filtered and concentrated. The residue was purified by chromatography on silica-gel with EA/PE (1:4) to afford product (63 g, 70.9% yield) as a faint yellow solid. Mass (m/z): 356 [M+H]⁺. ¹HNMR (400 MHZ, CDCl₃) δ 8.04-8.06 (m, 1H), 7.55-7.59 (t, J=8.0 Hz, 1H), 7.27-7.30 (t, J=6.0 Hz, 1H), 4.28 (s, 2H), 4.11-4.14 (m, 2H), 3.73-3.76 (m, 2H), 1.49 (s, 9H).

Step 2. Preparation of tert-butyl 3-oxo-4-(6-(4,4,5,5-tetramethyl-1,3,2-dioxaborolan-2-yl)pyridin-2-yl)piperazine-1-carboxylate: To a solution of tert-butyl 4-(6-bromopyridin-2-yl)-3-oxopiperazine-1-carboxylate (5.0 g, 14.1 mmol, 1.0 eq), 4,4,5,5-tetramethyl-2-(4,4,5,5-tetramethyl-1,3,2-dioxaborolan-2-yl)-1,3,2-dioxaborolane (10.0 g, 39.5 mmol, 2.8 eq) and KOAc (4.14 g, 42.3 mmol, 3.0 eq) in dioxane (150 mL) under N₂ was added Pd(dppf)Cl₂ (0.52 g, 0.705 mmol, 0.05 eq). The reaction mixture was stirred at 110° C. for 1.5 hrs. The reaction was filtered and concentrated. The residue was slurry with PE, filtered. The filtrate was concentrated to afford product (1.8 g, 39.8% yield) as a yellow oil. Mass (m/z): 322.1 [M+H−82]⁺.

Step 3. Preparation of tert-butyl 4-(6-(4-chloro-3-cyclopropyl-1H-pyrrolo[2,3-b]pyridin-5-yl)pyridin-2-yl)-3-oxopiperazine-1-carboxylate: Following General Step A, product tert-butyl 4-(6-(4-chloro-3-cyclopropyl-1H-pyrrolo[2,3-b]pyridin-5-yl)pyridin-2-yl)-3-oxopiperazine-1-carboxylate (800 mg, 33.7% yield) was obtained as a yellow oil. Mass (m/z): 468.1 [M+H]⁺.

Step 4. Preparation of 1-(6-(4-chloro-3-cyclopropyl-1H-pyrrolo[2,3-b]pyridin-5-yl)pyridin-2-yl)piperazin-2-one: Following General Step B1, product 1-(6-(4-chloro-3-cyclopropyl-1H-pyrrolo[2,3-b]pyridin-5-yl)pyridin-2-yl)piperazin-2-one (4.70 g, 67.2% yield) was obtained as a yellow semi-solid. Mass (m/z): 367 [M+H]⁺. ¹HNMR (400 MHZ, CD₃OD) δ 8.26 (s, 1H), 7.88-7.92 (m, 1H), 7.78-7.80 (m, 1H), 7.56-7.58 (m, 1H), 7.14-7.15 (m, 1H), 4.00-4.03 (m, 2H), 3.60 (s, 2H), 3.16-3.19 (m, 2H), 2.21-2.25 (m, 1H), 0.86-0.91 (m, 2H), 0.61-0.65 (m, 2H).

### Intermediate F: 1-(3-(4-chloro-3-(pyridin-2-ylethynyl)-1H-pyrrolo[2,3-b]pyridin-5-yl)phenyl)piperazin-2-one

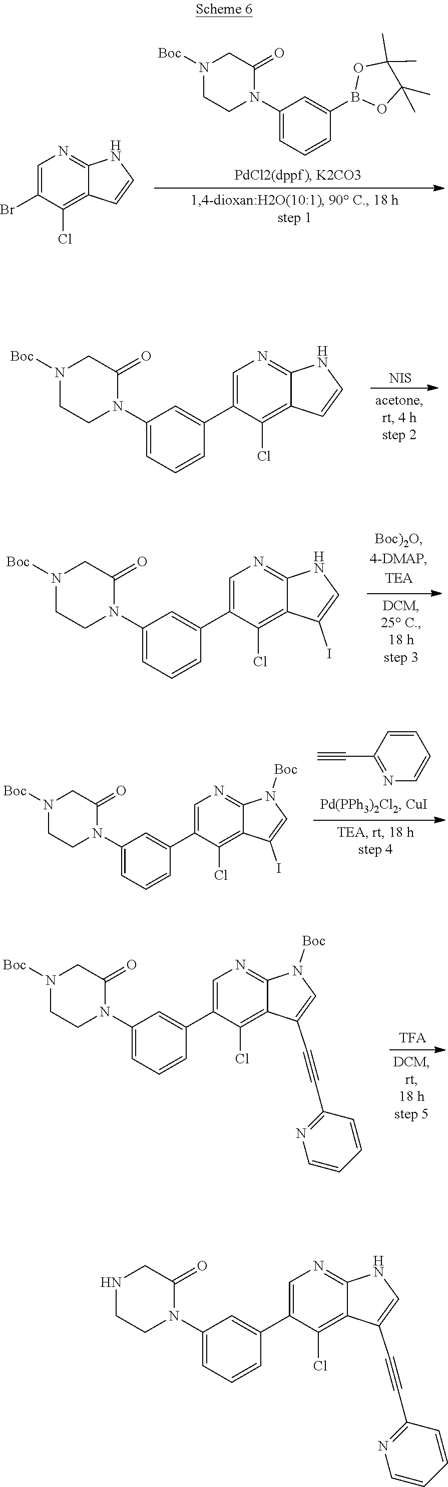

Step 1. Preparation of tert-butyl 4-(3-(4-chloro-1H-pyrrolo[2,3-b]pyridin-5-yl)phenyl)-3-oxopiperazine-1-carboxylate: Following General Step A, tert-butyl 4-(3-(4-chloro-1H-pyrrolo[2,3-b]pyridin-5-yl)phenyl)-3-oxopiperazine-1-carboxylate was prepared as a brown solid (15 g, 77% yield). MS: m/z=426.9. (M+1, ESI+).

Step 2. Preparation of tert-butyl 4-(3-(4-chloro-3-iodo-1H-pyrrolo[2,3-b]pyridin-5-yl)phenyl)-3-oxopiperazine-1-carboxylate: To a solution of tert-butyl [4-(3-{4-chloro-7H-pyrrolo[2,3-b]pyridin-3-yl}phenyl)-3-oxopiperazin-1-yl]formate (15 g, 35.1 mmol) in acetone (300 mL) was added NIS (8.69 g, 38.6 mmol). The resulting mixture was stirred at 25° C. for 4 hrs, and then concentrated. The residue was diluted with EA (200 mL), washed with water (100 mL×2), dried over Na₂SO₄, filtered and evaporated. The residue was purified by column chromatography (EA:PE=1:1) to give tert-butyl 4-(3-(4-chloro-3-iodo-1H-pyrrolo[2,3-b] pyridin-5-yl)phenyl)-3-oxopiperazine-1-carboxylate (8 g, 39% yield) as a yellow solid. MS: m/z=552.7. (M+1, ESI+).

Step 3. Preparation of tert-butyl 5-(3-(4-(tert-butoxy carbonyl)-2-oxopiperazin-1-yl)phenyl)-4-chloro-3-iodo-1H-pyrrolo[2,3-b]pyridine-1-carboxylate: A mixture solution of tert-butyl 4-(3-(4-chloro-3-iodo-1H-pyrrolo[2,3-b]pyridin-5-yl)phenyl)-3-oxopiperazine-1-carboxylate (5 g, 9 mmol), 4-DMAP (0.11 g, 0.9 mmol), (Boc)₂O (2.95 g, 13.5 mmol) and TEA (01.82 g, 18 mmol) in DCM (50 mL) was stirred under nitrogen at RT overnight. The mixture was concentrated, and the residue was purified by column chromatography (eluting with EA:PE=1:1) to give tert-butyl 5-(3-(4-(tert-butoxy carbonyl)-2-oxopiperazin-1-yl)phenyl)-4-chloro-3-iodo-1H-pyrrolo[2,3-b]pyridine-1-carboxylate (2.5 g, 40%) as a yellow solid. MS: m/z=652.7 (M+1, ESI+).

Step 4. Preparation of tert-butyl 5-(3-(4-(tert-butoxy carbonyl)-2-oxopiperazin-1-yl)phenyl)-4-chloro-3-(pyridin-2-ylethynyl)-1H-pyrrolo[2,3-b]pyridine-1-carboxylate: A mixture solution of tert-butyl 5-(3-(4-(tert-butoxy carbonyl)-2-oxopiperazin-1-yl)phenyl)-4-chloro-3-iodo-1H-pyrrolo[2,3-b]pyridine-1-carboxylate (208.9 mg, 0.32 mmol), 2-ethynylpyridine (164.8 mg, 1.6 mmol), Bis(triphenylphosphine) palladium (II) chloride (22.4 mg, 0.032 mmol), and Copper (I) iodide (12.2 mg, 0.064 mmol) in TEA (2 mL) and DMF (2 mL) was stirred under nitrogen at RT for 18 hrs. Methyl tert-butyl ether was added. A white solid precipitated out and was filtered. The residue was purified by column chromatography (MeOH:DCM=1:10) to give crude product, which was further purified by Prep-HPLC (xbridge-c18 150×19 mm, 5 um, mobile term: ACN-H₂O (0.1% FA), gradient: 20-40) to give tert-butyl 5-(3-(4-(tert-butoxy carbonyl)-2-oxopiperazin-1-yl)phenyl)-4-chloro-3-(pyridin-2-ylethynyl)-1H-pyrrolo[2,3-b] pyridine-1-carboxylate as a grey solid (150 mg, 74%). MS: m/z=627.8 (M+1, ESI+).

Step 5. Preparation of 1-(3-(4-chloro-3-(pyridin-2-ylethynyl)-1H-pyrrolo[2,3-b] pyridin-5-yl)phenyl)piperazin-2-one: A solution of tert-butyl 5-(3-(4-(tert-butoxy carbonyl)-2-oxopiperazin-1-yl)phenyl)-4-chloro-3-(pyridin-2-ylethynyl)-1H-pyrrolo[2,3-b]pyridine-1-carboxylate (165 mg, 0.27 mmol) in DCM (5 mL) and TFA (5 mL) was stirred at rt for 18 rs. After concentration, the residue was purified by Prep-HPLC (xbridge-c18 150×19 mm, 5 um, mobile term: ACN-H₂O (0.1% TA), gradient: 10-40) to give 1-(3-(4-chloro-3-(pyridin-2-ylethynyl)-1H-pyrrolo[2,3-b]pyridin-5-yl)phenyl)piperazin-2-one as a gray solid (80 mg, 71%). MS: m/z=427.8 (M+1, ESI+).

### Intermediate G: tert-butyl 4-(2-(2,6-dioxopiperidin-3-yl)-1-oxoisoindolin-5-yl)piperidine-1-carboxylate

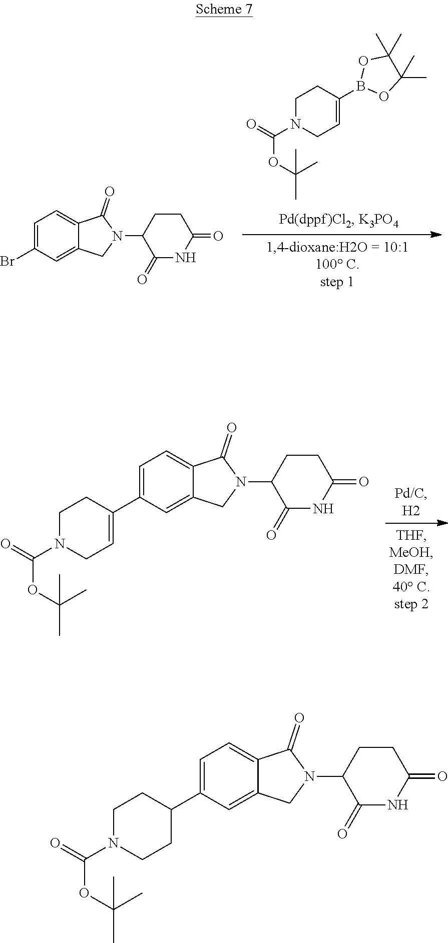 [SMILES: O=C1CCC(N2Cc3cc(Br)ccc3C2=O)C(=O)N1 ; CC(C)(C)OC(=O)N1CC=C(B2OC(C)(C)C(C)(C)O2)CC1 ; CC(C)(C)OC(=O)N1CC=C(c2ccc3c(c2)CN(C2CCC(=O)NC2=O)C3=O)CC1 ; CC(C)(C)OC(=O)N1CCC(c2ccc3c(c2)CN(C2CCC(=O)NC2=O)C3=O)CC1]

Step 1. tert-butyl 4-(2-(2,6-dioxopiperidin-3-yl)-1-oxoisoindolin-5-yl)-3,6-dihydropyridine-1 (2H)-carboxylate: To a mixture of 3-(5-bromo-1-oxo-3H-isoindol-2-yl)piperidine-2,6-dione (1 g, 3.1 mmol), tert-butyl [4-(4,4,5,5-tetramethyl-1,3,2-dioxaborolan-2-yl)-3,6-dihydro-2H-pyridin-1-yl]formate (1.92 g, 6.2 mmol) and K₃PO₄ (1.32, 6.2 mmol) in dioxane/H2O (10:1, 20 mL) under N₂ was added Pd(dppf)Cl₂ (0.23 g, 0.3 mmol). The reaction mixture was stirred at 100° C. for 18 hrs. The reaction was filtered and concentrated. The residue was purified by combi-flash with EA/PE (1:2) to afford tert-butyl 4-(2-(2,6-dioxopiperidin-3-yl)-1-oxoisoindolin-5-yl)-3,6-dihydropyridine-1 (2H)-carboxylate (1 g, yield: 70.97%) as a brown solid. Mass (m/z): 425.9 [M+H]⁺.

Step 2. tert-butyl 4-(2-(2,6-dioxopiperidin-3-yl)-1-oxoisoindolin-5-yl)piperidine-1-carboxylate: To a solution of tert-butyl {4-[2-(2,6-dioxopiperidin-3-yl)-1-oxo-3H-isoindol-5-yl]-3,6-dihydro-2H-pyridin-1-yl}formate (500 mg, 1.1724 mmol) in DMF (50 mL), MeOH (50 mL) and THF (50 mL) was added Pd(OH)₂/C (65.86 mg, 0.4689 mmol) and AcOH (2 mL). Then the reaction was stirred for 18 h at 40° C. under an atmosphere of H₂. Suction filtration and vacuum concentration to give tert-butyl 4-(2-(2,6-dioxopiperidin-3-yl)-1-oxoisoindolin-5-yl)piperidine-1-carboxylate (500 mg, 94.55%) as a black oil. Mass (m/z): 499.9 [M+H]⁺.

### Intermediate H: tert-butyl 4-(2-(2,6-dioxopiperidin-3-yl)-1,3-dioxoisoindolin-5-yl)piperidine-1-carboxylate

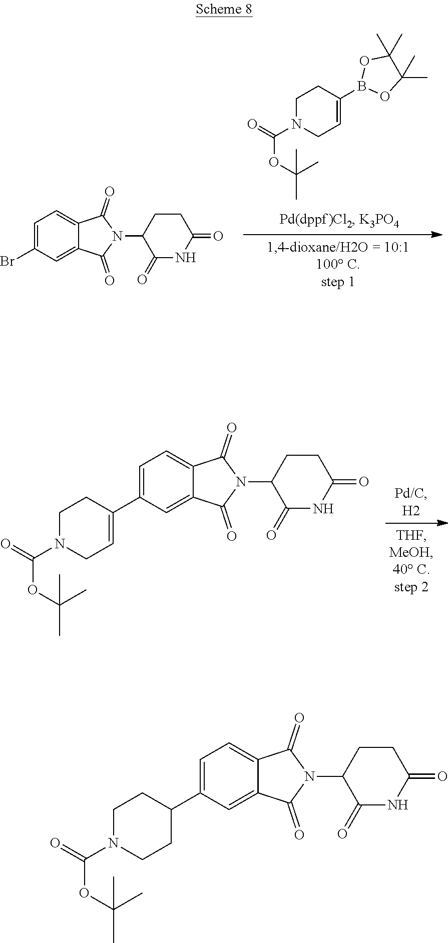 [SMILES: O=C1CCC(N2C(=O)c3ccc(Br)cc3C2=O)C(=O)N1 ; CC(C)(C)OC(=O)N1CC=C(B2OC(C)(C)C(C)(C)O2)CC1 ; CC(C)(C)OC(=O)N1CC=C(c2ccc3c(c2)C(=O)N(C2CCC(=O)NC2=O)C3=O)CC1 ; CC(C)(C)OC(=O)N1CCC(c2ccc3c(c2)C(=O)N(C2CCC(=O)NC2=O)C3=O)CC1]

Step 1. tert-butyl 4-(2-(2,6-dioxopiperidin-3-yl)-1,3-dioxoisoindolin-5-yl)-3,6-dihydropyridine-1 (2H)-carboxylate: To a mixture of 5-bromo-2-(2,6-dioxopiperidin-3-yl) isoindole-1,3-dione (1 g, 3 mmol), tert-butyl [4-(4,4,5,5-tetramethyl-1,3,2-dioxaborolan-2-yl)-3,6-dihydro-2H-pyridin-1-yl]formate (1.12 g, 3.6 mmol) and K₃PO₄ (0.76 g, 3.6 mmol) in dioxane/H₂O (10:1, 20 mL) under N₂ was added Pd(dppf)Cl₂ (0.12 g, 0.1 mmol). The reaction mixture was stirred at 90° C. for 18 hrs. 5% citric acid water (20 mL) was added and extracted with DCM (20 mL×2). The DCM layer was washed with brine (20 mL×2) dried over Na₂SO₄ and concentrated, the residue was purified by combi-flash with MeOH:DCM (1:10) to give the product (1 g, 73.33%) as brown solid: Mass (m/z): 462 [M+Na]⁺.

Step 2. tert-butyl 4-(2-(2,6-dioxopiperidin-3-yl)-1,3-dioxoisoindolin-5-yl)piperidine-1-carboxylate: To a solution of tert-butyl 4-[2-(2,6-dioxopiperidin-3-yl)-1,3-dioxoisoindol-5-yl]-3,6-dihydro-2H-pyridine-1-carboxylate (500 mg, 1.1378 mmol) in MeOH (10 mL) and THF (10 mL) was added Pd/C (48.43 mg, 0.4551 mmol). Then the reaction was stirred for 18 hrs at 40° C. under an atmosphere of H₂. By suction filtration, the filtrated was concentrated under vacuum to give the product (400 mg, 75.65%) as a gray solid. Mass (m/z): 464 [M+Na]⁺.

### Intermediate I: 2-(2,6-dioxopiperidin-3-yl)-5-(piperazin-1-yl)isoindoline-1,3-dione

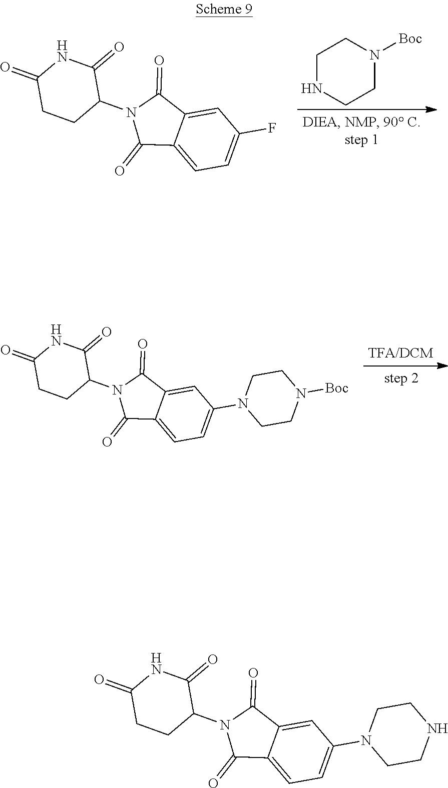 [SMILES: O=C1CCC(N2C(=O)c3ccc(F)cc3C2=O)C(=O)N1 ; CC(C)(C)OC(=O)N1CCNCC1 ; CC(C)(C)OC(=O)N1CCN(c2ccc3c(c2)C(=O)N(C2CCC(=O)NC2=O)C3=O)CC1 ; O=C1CCC(N2C(=O)c3ccc(N4CCNCC4)cc3C2=O)C(=O)N1]

Step 1. Preparation of tert-butyl {4-[2-(2,6-dioxopiperidin-3-yl)-1,3-dioxoisoindol-5-yl]piperazin-1-yl}formate: To a solution of 2-(2,6-dioxopiperidin-3-yl)-5-fluoroisoindole-1,3-dione (1.5 g, 5.4 mmol) in NMP (20 mL) was added tert-butyl piperazin-1-yl formate (1.01 g, 5.4 mmol) and DIEA (2.09 g, 0.016 mol). The reaction mixture was stirred at 90° C. under N₂ for 6 hrs. The reaction solution was extracted with EA (100 mL×3). The combined organic layer was washed with brine (30 mL×3), then dried over with anhydrous Na₂SO₄. After filtration, the solution was concentrated under vacuum, and the crude product was purified by Combiflash (PE/EA=1:1) to give the product as yellow solid (1.8 g, 72%). Mass (m/z): 464.6 [M+Na]⁺.

### Step 2. Preparation of 2-(2,6-dioxopiperidin-3-yl)-5-(piperazin-1-yl) isoindole-1,3-dione

To a solution of tert-butyl {4-[2-(2,6-dioxopiperidin-3-yl)-1,3-dioxoisoindol-5-yl]piperazin-1-yl}formate (1.8 g, 4.06 mmol) in DCM (10 mL) was added TFA (5 mL). The reaction mixture was stirred at room 25° C. for 2 hrs. The solvent was removed under reduced pressure and freeze dried to give the crude product as a yellow solid (1.2 g, 93%). Mass (m/z): 426.7 [M+H]+.

### Intermediate J: 3-(1-oxo-5-(piperazin-1-yl) isoindolin-2-yl)piperidine-2,6-dione

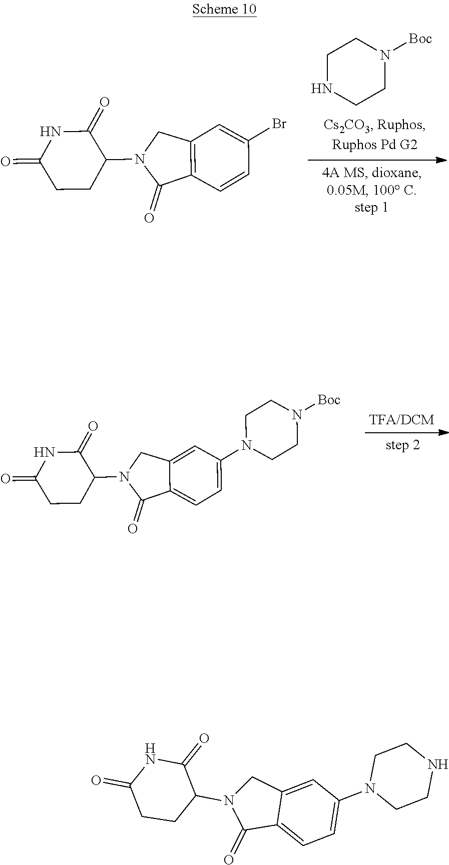 [SMILES: O=C1CCC(N2Cc3cc(Br)ccc3C2=O)C(=O)N1 ; CC(C)(C)OC(=O)N1CCNCC1 ; CC(C)(C)OC(=O)N1CCN(c2ccc3c(c2)CN(C2CCC(=O)NC2=O)C3=O)CC1 ; O=C1CCC(N2Cc3cc(N4CCNCC4)ccc3C2=O)C(=O)N1]

Step 1. Preparation of tert-butyl 4-(2-(2,6-dioxopiperidin-3-yl)-1-oxoisoindolin-5-yl)piperazine-1-carboxylate: To a solution of 3-(5-bromo-1-oxoisoindolin-2-yl)piperidine-2,6-dione (10 g, 0.031 mol) tert-butyl piperazine-1-carboxylate (20.25 g, 0.11 mol), Cs₂CO₃ (30.2 g, 0.093 mol), Ruphos (2.88 g, 0.006 mol) and 4 A molecular sieves (410 mg, 0.9 mmol) in dioxane (600 mL) was added RuPhos Pd G2 (4.8 g, 0.006 mol). The reaction mixture was stirred at 100° C. under N₂ for 48 hrs. After the reaction completed, H₂O (1000 mL) was added to the reaction mixture, and then extracted with DCM (500 mL×3). The combined organic layer was washed with brine (500 mL×2), then dried over with anhydrous Na₂SO₄. After filtration, the solution was concentration under vacuum, and the residue was purified by Combiflash (DCM/MeOH=0˜10%) to give the product tert-butyl 4-(2-(2,6-dioxopiperidin-3-yl)-1-oxoisoindolin-5-yl)piperazine-1-carboxylate as a yellow solid (6.7 g, 50%). Mass (m/z): 429.2 [M+H]⁺.

Step 2. 3-(1-oxo-5-(piperazin-1-yl) isoindolin-2-yl)piperidine-2,6-dione: To a solution of tert-butyl {4-[2-(2,6-dioxopiperidin-3-yl)-1-oxo-3H-isoindol-5-yl]piperazin-1-yl}formate (700 mg, 1.62 mmol) in DCM (15 mL) was added TFA (3 mL). The reaction mixture was stirred at rt under N₂ for 18 hrs. Methyl tert butyl ether (10 mL) was added to precipitate brown solid, and the desired product was obtained by suction filtration (500 mg, 88%) as a brown solid. Mass (m/z): 329 [M+H]⁺.

### Intermediate K: tert-butyl 4-(5-(2,4-dioxotetrahydropyrimidin-1 (2H)-yl) naphthalen-2-yl)piperazine-1-carboxylate

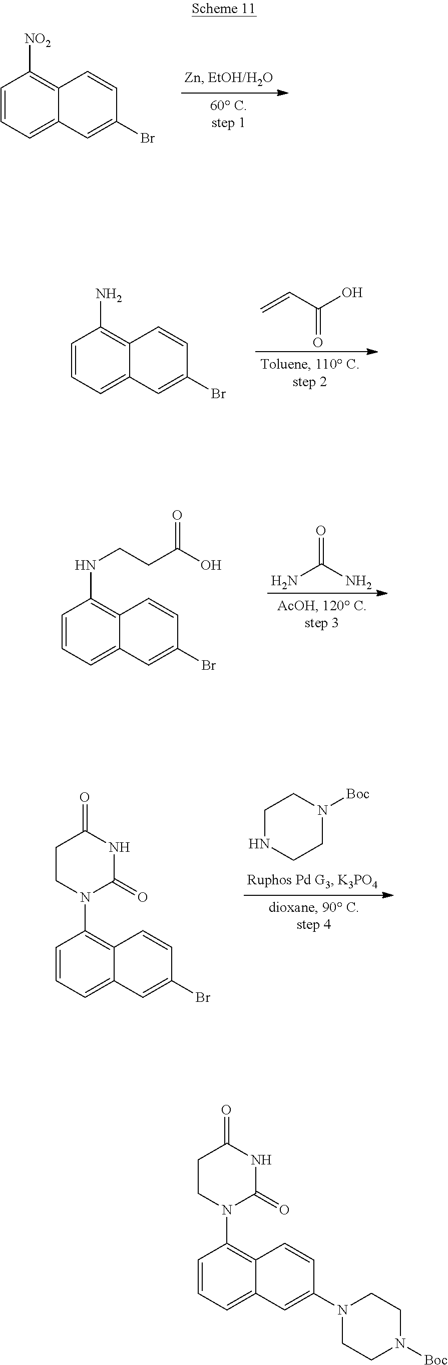

Step 1. Preparation of 6-bromonaphthalen-1-amine: To a mixture of 6-bromo-1-nitronaphthalene (5.00 g, 0.0198 mol) in EtOH/H₂O (3:1, 50 mL) was added NH₄Cl (7.73 g, 0.145 mol). The reaction mixture was warmed to 60° C. and then Zn powder (9.45 g, 0.145 mol) was added portion wise. The reaction mixture was stirred at 60° C. for 1 hr. The reaction mixture was filtered and the filtrate was concentrated under reduced pressure. The residue was diluted with water (200 mL) then extracted with EtOAc (150 mL×3), washed with brine (200 mL), dried over Na₂SO₄ and concentrated under reduced pressure. The crude product was purified by Flash Chromatography (PE/EA=0˜40%) to give the product 6-bromonaphthalen-1-amine as a brown solid (3.87 g, 83%). Mass (m/z): 221.9 [M+H]⁺.

Step 2. Preparation of 3-[(6-bromonaphthalen-1-yl)amino]propanoic acid: To a mixture of 6-bromonaphthalen-1-amine (3.87 g, 0.0174 mol) in toluene (40.0 mL) was added prop-enoic acid (7.52 g, 0.104 mol). The reaction was degassed with N₂ and stirred at 110° C. for 21 hrs. The reaction mixture was concentrated under reduce pressure to the product 3-[(6-bromonaphthalen-1-yl)amino]propanoic acid as a brown solid (8.00 g, 93%). Mass (m/z): 293.9 [M+H]⁺.

Step 3. Preparation of 1-(6-bromonaphthalen-1-yl)-1,3-diazinane-2,4-dione: To a mixture of 3-[(6-bromonaphthalen-1-yl)amino]propanoic acid (8.7 g, 0.0296 mol) in AcOH (180 mL) was added urea (4.44 g, 0.0740 mol). The reaction mixture was stirred at 120° C. under N₂ for 16 hrs. The reaction mixture was poured into water (200 mL) slowly at 0° C. and then filtered. The cake was dried under reduced pressure to give the product 1-(6-bromonaphthalen-1-yl)-1,3-diazinane-2,4-dione as brown solid (7.5 g, 71%). Mass (m/z): 318.8 [M+H]⁺.

Step 4. Preparation of tert-butyl {4-[5-(2,4-dioxo-1,3-diazinan-1-yl) naphthalen-2-yl]piperazin-1-yl}formate: To a mixture of 1-(6-bromonaphthalen-1-yl)-1,3-diazinane-2,4-dione (500 mg, 1.57 mmol) in dioxane (10.0 mL) was added K₃PO₄ (998 mg, 4.70 mmol), tert-butyl piperazin-1-yl-formate (586 mg, 3.133 mmol) and RuPhos Pd G3 (262 mg, 0.313 mmol). The reaction was degassed with N₂ for 3 times and stirred at 90° C. for 16 hrs. The reaction mixture was filtered and the filtrate was concentrated under reduced pressure. The residue was diluted with EA (25 mL) and H₂O (50 mL), then extracted with EA (50 mL×3), washed with brine (100 mL), dried over Na₂SO₄ and concentrated under reduced pressure. The crude product was purified by Flash Chromatography (PE/EA=0˜30%) to give the product tert-butyl {4-[5-(2,4-dioxo-1,3-diazinan-1-yl) naphthalen-2-yl]piperazin-1-yl}formate as a white solid (360 mg, 49%). Mass (m/z): 424.9 [M+H]⁺.

### Intermediate L: 1-[6-(piperidin-4-yl) naphthalen-1-yl]-1,3-diazinane-2,4-dione

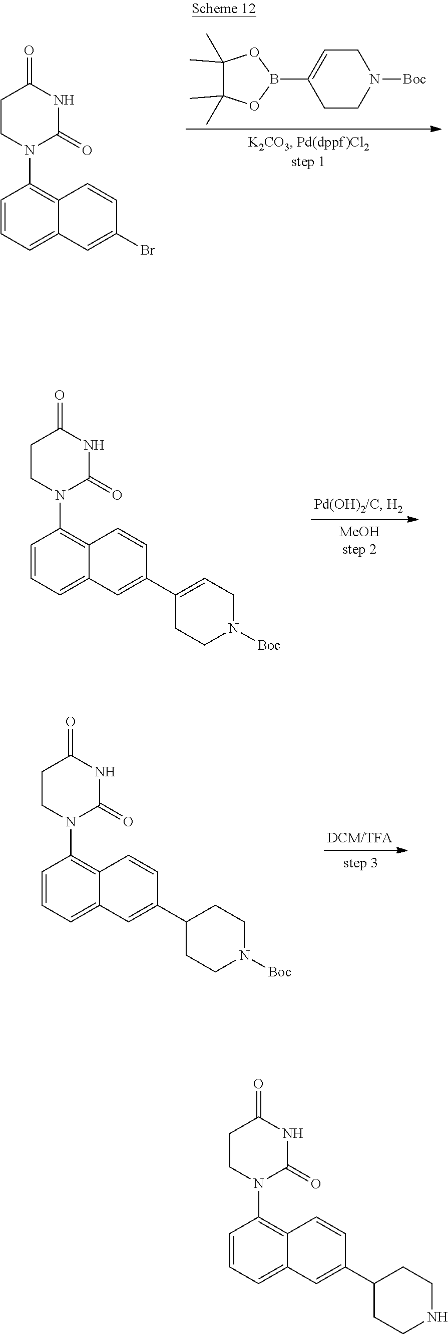 [SMILES: O=C1CCN(c2cccc3cc(Br)ccc23)C(=O)N1 ; CC(C)(C)OC(=O)N1CC=C(B2OC(C)(C)C(C)(C)O2)CC1 ; CC(C)(C)OC(=O)N1CC=C(c2ccc3c(N4CCC(=O)NC4=O)cccc3c2)CC1 ; CC(C)(C)OC(=O)N1CCC(c2ccc3c(N4CCC(=O)NC4=O)cccc3c2)CC1 ; O=C1CCN(c2cccc3cc(C4CCNCC4)ccc23)C(=O)N1]

Step 1. Preparation of tert-butyl {4-[5-(2,4-dioxo-1,3-diazinan-1-yl) naphthalen-2-yl]-3,6-dihydro-2H-pyridin-1-yl}formate: To mixture of 1-(6-bromonaphthalen-1-yl)-1,3-diazinane-2,4-dione (20.0 g, 0.063 mol) in dioxane/H₂O (10:1, 400 mL) was added K₃PO₄ (26.6 g, 0.125 mol), tert-butyl [4-(4,4,5,5-tetramethyl-1,3,2-dioxaborolan-2-yl)-3,6-dihydro-2H-pyridin-1-yl] formate (23.3 g, 0.075 mol) and Pd(dppf)Cl₂ (2.29 g, 0.003 mol). The reaction was degassed with N₂ and stirred at 100° C. for 16 hrs. The reaction mixture was filtered and the filtrate was concentrated under reduced pressure. The residue was diluted with water (100 mL), then extracted with EA (500 mL×3). The organic layer was washed with brine (300 mL), dried over Na₂SO₄ and concentrated under reduced pressure. The residue was purified by column flash (PE/EA=0˜80%) to give the product tert-butyl {4-[5-(2,4-dioxo-1,3-diazinan-1-yl) naphthalen-2-yl]-3,6-dihydro-2H-pyridin-1-yl}formate (12.0 g, 43%) was a yellow solid. Mass (m/z): 366.2 [M−55]⁺.

Step 2. Preparation of tert-butyl {4-[5-(2,4-dioxo-1,3-diazinan-1-yl) naphthalen-2-yl]piperidin-1-yl}formate: To a mixture of tert-butyl {4-[5-(2,4-dioxo-1,3-diazinan-1-yl) naphthalen-2-yl]-3,6-dihydro-2H-pyridin-1-yl}formate (8.0 g, 0.019 mol) in MeOH (500 mL) was added Pd(OH)₂/C (1.6 g, 20% wt/wt). The reaction was stirred at 25° C. under H₂ for 16 hrs. The reaction mixture was filtered and the filtrate was concentrated under reduced pressure to give the product tert-butyl {4-[5-(2,4-dioxo-1,3-diazinan-1-yl) naphthalen-2-yl]piperidin-1-yl}formate (6.8 g, 76%) as a yellow solid. Mass (m/z): 368.2 [M−55]⁺.

Step 3. Preparation of 1-[6-(piperidin-4-yl) naphthalen-1-yl]-1,3-diazinane-2,4-dione: A solution of tert-butyl {4-[5-(2,4-dioxo-1,3-diazinan-1-yl) naphthalen-2-yl]piperidin-1-yl}formate (6.8 g, 0.016 mol) in DCM/TFA (3:1, 100 mL) was stirred at 25° C. for 3 hrs. The reaction mixture was concentrated under reduced pressure. The residue was triturated with MTBE (100 mL) and filtered. The filter cake was dried under reduced pressure to give the product 1-[6-(piperidin-4-yl) naphthalen-1-yl]-1,3-diazinane-2,4-dione (6.0 g, 99%) as an off white solid. Mass (m/z): 324.2 [M+H]⁺.

### Intermediate M: tert-butyl 4-(4-(2,6-dioxopiperidin-3-yl)phenyl)piperidine-1-carboxylate

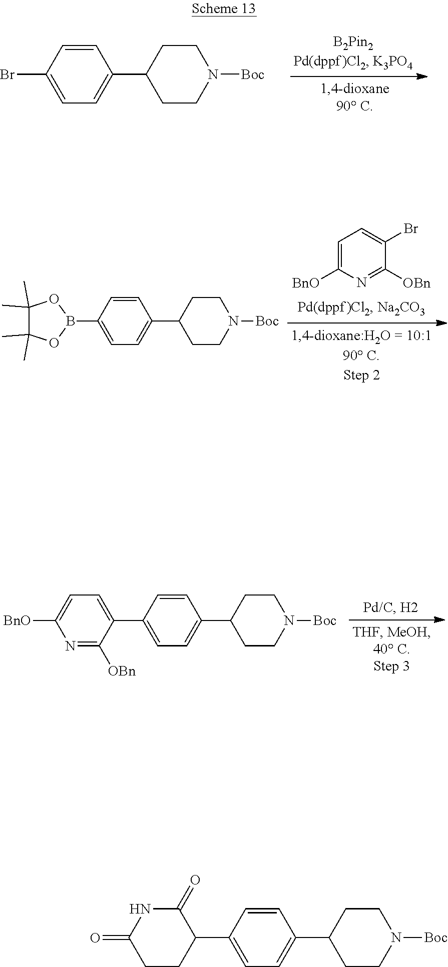 [SMILES: CC(C)(C)OC(=O)N1CCC(c2ccc(Br)cc2)CC1 ; CC(C)(C)OC(=O)N1CCC(c2ccc(B3OC(C)(C)C(C)(C)O3)cc2)CC1 ; Brc1ccc(OCc2ccccc2)nc1OCc1ccccc1 ; CC(C)(C)OC(=O)N1CCC(c2ccc(-c3ccc(OCc4ccccc4)nc3OCc3ccccc3)cc2)CC1 ; CC(C)(C)OC(=O)N1CCC(c2ccc(C3CCC(=O)NC3=O)cc2)CC1]

Step 1. tert-butyl 4-(4-(4,4,5,5-tetramethyl-1,3,2-dioxaborolan-2-yl)phenyl) piperidine-1-carboxylate: To a mixture of [4-(4-bromophenyl)piperidin-1-yl] tert-butyl formate (1 g, 2.9 mmol), tert-butyl [4-(4,4,5,5-tetramethyl-1,3,2-dioxaborolan-2-yl)-3,6-dihydro-2H-pyridin-1-yl]formate (1.47 g, 5.8 mmol) and KOAc (0.85 g, 8.7 mmol) in dioxane (20 mL) under N₂ was added Pd(dppf)Cl₂ (0.2 g, 0.2 mmol). The reaction mixture was stirred at 90° C. for 18 hrs. 5% citric acid water (20 mL) was added and extracted with DCM (20 mL×2). The organic layer was washed with brine (20 mL×2) dried over Na₂SO₄ and concentrated, the residue was purified by combi-flash with MeOH/DCM (1:10) to give the product (0.8 g, 68.97%) as brown solid: Mass (m/z): 410 [M+Na]⁺.

Step 2. tert-butyl 4-(4-(2,6-bis(benzyloxy)pyridin-3-yl)phenyl)piperidine-1-carboxylate: To a mixture of 2,6-bis(benzyloxy)-3-bromopyridine (500 mg, 1.3505 mmol), tert-butyl {4-[4-(4,4,5,5-tetramethyl-1,3,2-dioxaborolan-2-yl)phenyl]piperidin-1-yl}formate (786.66 g, 2.0257 mmol) and Na₂CO₃ (286.27 mg, 2.701 mmol) in dioxane/H2O (10:1, 10 mL) under N₂ was added Pd(dppf)Cl₂ (49.41 mg, 0.0675 mmol). The reaction mixture was stirred at 90° C. for 18 hrs. 5% citric acid water (20 mL) was added and extracted with DCM (20 mL×2). The organic layer was washed with brine (20 mL×2) dried over Na₂SO₄ and concentrated, the residue was purified by combi-flash with MeOH/DCM (1:10) to afford target compound (500 mg, yield: 63.75%) as a brown solid. Mass (m/z): 550.9 [M+H]⁺.

Step 3. tert-butyl 4-(4-(2,6-dioxopiperidin-3-yl)phenyl)piperidine-1-carboxylate: To a solution of (4-{4-[2,6-bis(benzyloxy)pyridin-3-yl]phenyl}piperidin-1-yl) tert-butyl formate (250 mg, 0.4531 mmol) in MeOH (10 mL) and THF (10 mL) was added Pd/C (19.29 mg, 0.1812 mmol). Then the reaction was stirred for 18 h at 40° C. under an atmosphere of H₂. By suction filtration and vacuum concentration, the residue was purified by combi-flash with MeOH/DCM (1:10) to afford desired compound (400 mg, 75.65%) as a gray solid. Mass (m/z): 395 [M+Na]⁺.

### Intermediate N: tert-butyl 4-(4-(2,6-dioxopiperidin-3-yl)phenyl)piperazine-1-carboxylate

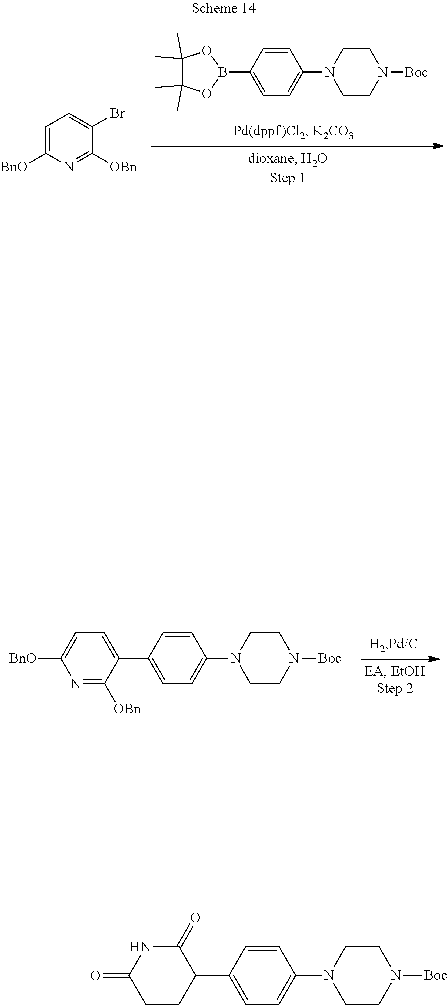 [SMILES: CC(C)(C)OC(=O)N1CCN(c2ccc(B3OC(C)(C)C(C)(C)O3)cc2)CC1 ; Brc1ccc(OCc2ccccc2)nc1OCc1ccccc1 ; CC(C)(C)OC(=O)N1CCN(c2ccc(-c3ccc(OCc4ccccc4)nc3OCc3ccccc3)cc2)CC1 ; CC(C)(C)OC(=O)N1CCN(c2ccc(C3CCC(=O)NC3=O)cc2)CC1]

Step 1. Preparation of tert-butyl 4-(4-(2,6-bis(benzyloxy)pyridin-3-yl)phenyl) piperazine-1-carboxylate: To a solution of 2,6-bis(benzyloxy)-3-bromopyridine (3 g, 8.10 mmol) and tert-butyl {4-[4-(4,4,5,5-tetramethyl-1,3,2-dioxaborolan-2-yl)phenyl]piperazin-1-yl}formate (3.15 g, 8.10 mmol) in dioxane (50 mL) and water (5 mL) was added K₂CO₃ (2.24 g, 16.20 mmol) and Pd(dppf)Cl₂ (593 mg, 0.81 mmol). The reaction mixture was stirred at 90° C. under N₂ for 16 hrs. The reaction mixture was concentrated and purified by silica gel column chromatography (PE/EA=3:1) to give the product as a yellow solid (4.6 g, 93%). Mass (m/z): 496.1 [M−55]⁺.

Step 2. Preparation of tert-butyl 4-(4-(2,6-dioxopiperidin-3-yl)phenyl)piperazine-1-carboxylate: To a solution of (4-{4-[2,6-bis(benzyloxy)pyridin-3-yl]phenyl}piperazin-1-yl) tert-butyl formate (4.5 g, 8.14 mmol) in EtOH (45 mL) and EA (45 mL) was added 10% Pd/C (1.73 g, 1.62 mmol). The reaction mixture was stirred at 25° C. under H₂ (1 atm) for 16 hrs. after filtration, the filtrate was concentrated to give the product as a white solid (2.2 g, 64%). Mass (m/z): 374.2 [M+H]⁺.

### Intermediate O: tert-butyl (4-{4-[(2,6-dioxopiperidin-3-yl)amino]-2-fluorophenyl}piperazin-1-yl) formate

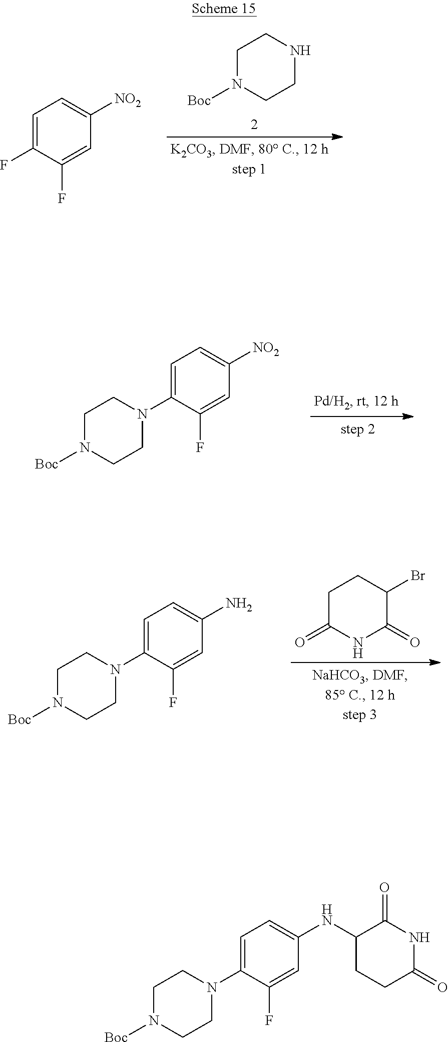 [SMILES: O=[N+]([O-])c1ccc(F)c(F)c1 ; CC(C)(C)OC(=O)N1CCNCC1 ; CC(C)(C)OC(=O)N1CCN(c2ccc([N+](=O)[O-])cc2F)CC1 ; CC(C)(C)OC(=O)N1CCN(c2ccc(N)cc2F)CC1 ; O=C1CCC(Br)C(=O)N1 ; CC(C)(C)OC(=O)N1CCN(c2ccc(NC3CCC(=O)NC3=O)cc2F)CC1]

Step 1. Preparation of tert-butyl [4-(2-fluoro-4-nitrophenyl)piperazin-1-yl]format: To a solution of 1,2-difluoro-4-nitrobenzene (5 g, 31.4 mmol) in DMF (50 mL) was added tert-butyl piperazin-1-yl formate (11.76 g, 62.8 mmol) and K₂CO₃ (8.68 g, 62.8 mmol). The reaction was stirred at 80° C. under N₂ for 12 h. The reaction mixture was quenched with ice water. The precipitated solid was filtered, dried under vacuum to afford the product as yellow solid (10 g, 93%). Mass (m/z): 326.1 [M+H]⁺.

Step 2. Preparation of [4-(4-amino-2-fluorophenyl)piperazin-1-yl] tert-butyl formate: To a solution of tert-butyl [4-(2-fluoro-4-nitrophenyl)piperazin-1-yl]formate (5 g, 15.3 mmol) in MeOH was added Pd/C (0.16 g, 1.5 mmol) under N₂ atmosphere. The suspension was degassed a nd purged with H₂ 3 times. the reaction was stirred at RT under H₂ for 24 h. The solution was filtered through celite, concentrated to afford the product as yellow solid (3 g, 63%). Mass (m/z): 296.1 [M+H]⁺.

Step 3. Preparation of tert-butyl (4-{4-[(2,6-dioxopiperidin-3-yl)amino]-2-fluorophenyl}piperazin-1-yl) formate: To a solution of [4-(4-amino-2-fluorophenyl)piperazin-1-yl] tert-butyl formate (500 mg, 1.69 mmol) in DMF (50 mL) was added 3-bromopiperidine-2,6-dione (972 mg, 5.06 mmol), NaHCO₃ (1417 mg, 16.9 mmol). The reaction was stirred at 85° C. for 12 h. The reaction mixture was quenched with ice water, extracted with EA (50 mL×3). The combined organic layer was washed with brine (30 mL×3), dried with anhydrous Na₂SO₄, filtered, and concentrated under reduced pressure. The residue was purified by flash chromatography (PE/EA=1:1) to give the product as white solid (300 mg, 41.8%). Mass (m/z): 407.2 [M+H]⁺.

### Intermediate P: tert-butyl (4-{4-[(2,6-dioxopiperidin-3-yl)amino]phenyl}piperidin-1-yl) formate

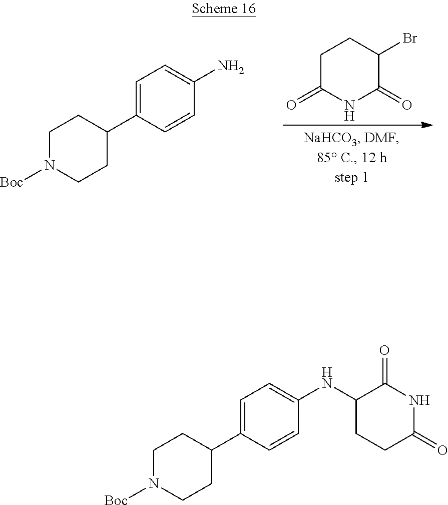 [SMILES: CC(C)(C)OC(=O)N1CCC(c2ccc(N)cc2)CC1 ; O=C1CCC(Br)C(=O)N1 ; CC(C)(C)OC(=O)N1CCC(c2ccc(NC3CCC(=O)NC3=O)cc2)CC1]

Step 1. Preparation of tert-butyl (4-{4-[(2,6-dioxopiperidin-3-yl)amino]phenyl}piperidin-1-yl) formate: To a solution of [4-(4-aminophenyl)piperidin-1-yl] tert-butyl formate (500 mg, 1.8 mmol) in DMF (5 mL) was added 3-bromopiperidine-2,6-dione (692 mg, 3.6 mmol), NaHCO₃ (1514 mg, 18 mmol). The reaction was stirred at 85° C. for 12 hrs. The reaction mixture was quenched with ice water, extracted with EA (50 mL×3). The combined organic layer was washed with brine (30 mL×3), dried with anhydrous Na₂SO₄, filtered, and concentrated under reduced pressure. The residue was purified by flash chromatography (PE/EA=1:1) to afford the product as white solid (300 mg, 39.8%). Mass (m/z): 388.2 [M+H]⁺.

### Intermediate Q: tert-butyl 4-(3-(2,4-dioxotetrahydropyrimidin-1 (2H)-yl)-1-methyl-1H-indazol-6-yl)piperidine-1-carboxylate

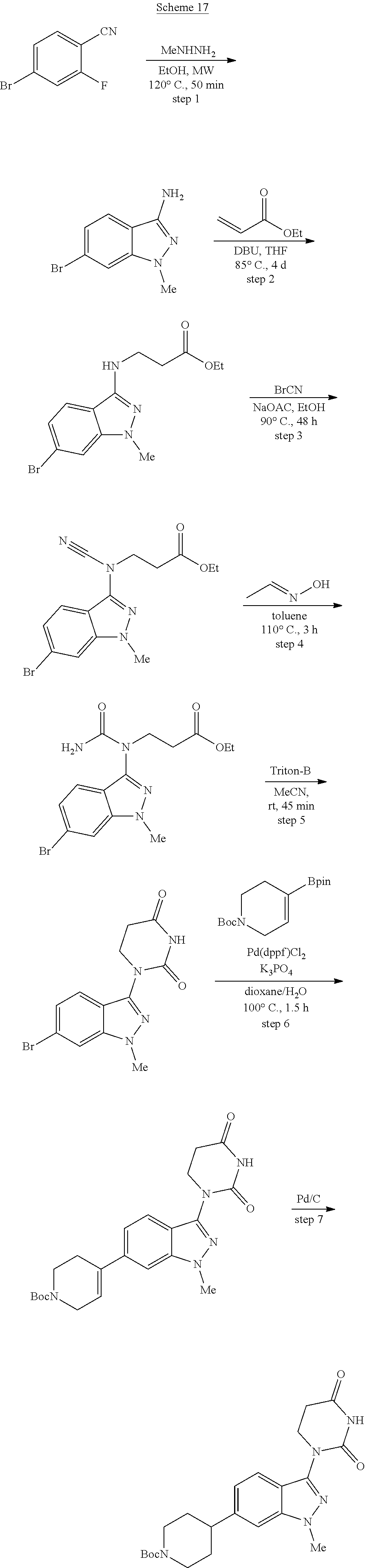

Step 1. Preparation of 6-bromo-1-methylindazol-3-amine: To a solution of 4-bromo-2-fluorobenzonitrile (3.0 g, 15 mmol) in EtOH (50 mL) was added methylhydrazine (2.1 g, 45 mm ol). The reaction was stirred for 50 min in microwave reactor (120° C.). The reaction mixture was quenched with ice water, extracted with EtOAc (50 mL×3). The combined organic layer was dried over anhydrous Na₂SO₄, filtered, concentrated under reduced pressure. The residue was purified by Flash Chromatography (PE/EA=2:1) to give the product as yellow solid (2.5 g, 70%). Mass (m/z): 227.9 [M+H]⁺.

Step 2. Preparation of ethyl 3-[(6-bromo-1-methylindazol-3-yl)amino]propanoate: To a solution of tert-butyl 6-bromo-1-methylindazol-3-amine (2.5 g, 11 mmol) in THF was added ethyl prop-2-enoate (11 g, 110 mmol) and DBU (16.9 g, 110 mmol). The reaction was stirred at 80° C. for 5 days. The reaction mixture was quenched with ice water, extracted with EtOAc (50 mL×3). The combined organic layer was dried over anhydrous Na₂SO₄, filtered, concentrated under reduced pressure. The residue was purified by flash chromatography (PE/EA=1:1) to give the product as yellow solid (1.2 g, 31%). Mass (m/z): 326.1 [M+H]⁺.

Step 3. Preparation of ethyl 3-[(6-bromo-1-methylindazol-3-yl)(cyano)amino]propanoate: To a solution of ethyl 3-[(6-bromo-1-methylindazol-3-yl)amino]propanoate (2, 1.1 g, 3.4 mm ol) in EtOH (20 mL) was added carbononitridic bromide (540 mg, 5 mmol), NaOAc (560 mg, 6.8 mmol). The reaction was stirred at 90° C. for 48 h. The reaction mixture was quenched with ice water, extracted with EA (50 mL×3). The combined organic layer was dried over anhydrous Na 2SO₄, filtered, concentrated under reduced pressure. The residue was purified by flash chromatography (PE/EA=1:1) to give the product (500 mg, 41%). Mass (m/z): 353.0 [M+H]⁺.

Step 4. Preparation of ethyl 3-[(6-bromo-1-methylindazol-3-yl)(carbamoyl)amino]propanoate: To a solution of ethyl 3-[(6-bromo-1-methylindazol-3-yl)(cyano)amino]propanoate (500 mg, 1.4 mmol) in toluene (10 mL) was added (E)-N-ethylidenehydroxylamine (252 mg, 4.2 m mol), InCl₃ (31 mg, 0.14 mmol). The reaction was stirred at 110° C. for 1 h. The reaction mixture was quenched with ice water, extracted with EA (50 mL×3). The combined organic layer was dried over anhydrous Na₂SO₄, filtered, concentrated under reduced pressure. The residue was purified by flash chromatography (PE/EA=1:1) to give the product as yellow solid (330 mg, 60%). Mass (m/z): 369.0 [M+H]⁺.

Step 5. Preparation of 1-(6-bromo-1-methylindazol-3-yl)-1,3-diazinane-2,4-dione: To a solution of ethyl 3-[(6-bromo-1-methylindazol-3-yl)(carbamoyl)amino]propanoate (330 mg, 0.9 mmol) in MeCN (5 mL) was added Benzyltrimethylammonium hydroxide (224 mg, 1.35 mmol). The reaction was stirred at room temperature for 1 h. The reaction mixture was quenched with ice water, extracted with EA (50 mL×3). The combined organic layer was dried over anhydrous Na₂SO₄, filtered, concentrated under reduced pressure. The residue was purified by flash chromatography (PE/EA=1:1) to give the product as white solid (247 mg, 82%). Mass (m/z): 323.0 [M+H]⁺.

Step 6. Preparation of tert-butyl {4-[3-(2,4-dioxo-1,3-diazinan-1-yl)-1-methylindazol-6-yl]-3,6-dihydro-2H-pyridin-1-yl}formate: To a solution of 1-(6-bromo-1-methylindazol-3-yl)-1,3-diazinane-2,4-dione (230 mg, 0.7 mmol) in 10:1 dioxane/H₂O (5 mL) was added tert-butyl [4-(4,4,5,5-tetramethyl-1,3,2-dioxaborolan-2-yl)-3,6-dihydro-2H-pyridin-1-yl]formate (441 mg, 1.4 mmol), K₃PO₄ (297 mg, 1.4 mmol) and Pd(dppf)Cl₂ (52 mg, 0.07 mmol). The solution was stirred at 100° C. under N₂ for 1.5 h. The reaction mixture was quenched with ice water, extracted with EA (10 mL×3). The combined organic layer was dried over anhydrous Na₂SO₄, filtered, concentrated under reduced pressure. The residue was purified by flash chromatography (PE/EA=1:1) to give product as white solid (200 mg, 63%). Mass (m/z): 426.2 [M+H]⁺.

Step 7. Preparation of tert-butyl {4-[3-(2,4-dioxo-1,3-diazinan-1-yl)-1-methylindazol-6-yl]piperidin-1-yl}formate: To a solution of tert-butyl {4-[3-(2,4-dioxo-1,3-diazinan-1-yl)-1-methylindazol-6-yl]-3,6-dihydro-2H-pyridin-1-yl}formate (100 mg, 0.2 mmol) in MeOH was added Pd/C (2.50 mg, 0.02 mmol) under N₂ atmosphere. The suspension was degassed and purged with H₂ 3 times. The reaction was stirred at RT under H₂ for 24 h. The reaction mixture was filtered and filtrate was concentrated to afford product as white solid (70 mg, 66%). Mass (m/z): 428.2 [M+H]⁺.

### Intermediate R: tert-butyl 4-(3-(2,6-dioxopiperidin-3-yl)-1-methyl-1H-indazol-6-yl)piperidine-1-carboxylate

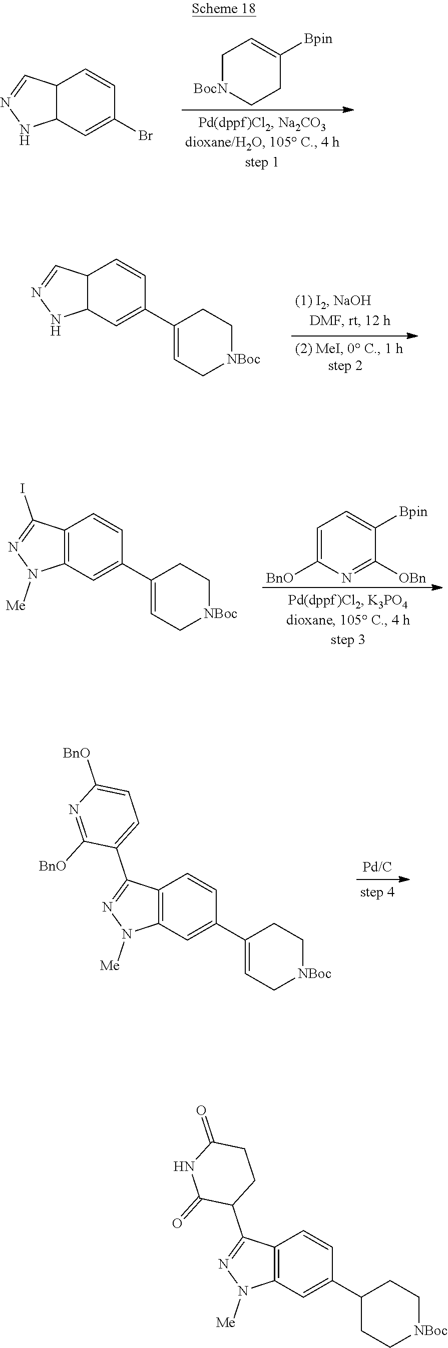

Step 1. Preparation of [4-(3a, 7a-dihydro-1H-indazol-6-yl)-3,6-dihydro-2H-pyridin-1-yl] tert-butyl formate: To a solution of 6-bromo-3a, 7a-dihydro-1H-indazole (6 g, 30 mmol) in 10:1 dioxane/H₂O (60 mL) was added tert-butyl [4-(4,4,5,5-tetramethyl-1,3,2-dioxaborolan-2-yl)-3,6-dihydro-2H-pyridin-1-yl]formate (14 g, 45 mmol), Pd(dppf)Cl₂ (2.5 g, 3 mmol) and Na₂CO₃ (6.4 g, 60 mmol). The reaction was stirred at 105° C. under N₂ for 12 h. The reaction mixture was quenched with ice water, extracted with EA (50 mL×3). The combined organic layer was dried over anhydrous Na₂SO₄, filtered, concentrated under reduced pressure. The residue was purified by flash chromatography (PE/EA=10:1) to give product as yellow solid (6 g, 63%). Mass (m/z): 300.2 [M+H].

Step 2. Preparation of tert-butyl [4-(3-iodo-1-methylindazol-6-yl)-3,6-dihydro-2H-pyridin-1-yl]formate: To a solution of [4-(3a, 7a-dihydro-1H-indazol-6-yl)-3,6-dihydro-2H-pyridin-1-yl] tert-butyl formate (7.5 g, 24.8 mmol) in DMF (50 mL) was added NaOH (3.0 g, 74.4 mmol) and I₂ (9.4 g, 37.2 mmol). The reaction was stirred at rt for 12 h. Mel (17.6 g, 124 mmol) was added. The reaction was stirred at 25° C. for another 1 h. The reaction mixture was quenched with ice water, extracted with EA (50 mL×3). The combined organic layer was dried over anhydrous Na₂SO₄, filtered, concentrated under reduced pressure. The residue was purified by flash chromatography (PE/EA=1:1) to give product as yellow solid (1 g, 9%). Mass (m/z): 440.1 [M+H]⁺.

Step 3. Preparation of (4-{3-[2,6-bis(benzyloxy)pyridin-3-yl]-1-methylindazol-6-yl}-3,6-dihydro-2H-pyridin-1-yl) tert-butyl formate: To a solution of tert-butyl [4-(3-iodo-1-methylindazol-6-yl)-3,6-dihydro-2H-pyridin-1-yl]formate (1 g, 2.3 mmol) in 10:1 dioxane/H₂O (10 mL) was added 2,6-bis(benzyloxy)-3-(4,4,5,5-tetramethyl-1,3,2-dioxaborolan-2-yl)pyridine (1.4 g, 3.5 mmol), Pd(dppf)Cl₂ (170 mg, 0.23 mmol) and Cs₂CO; (1.5 g, 4.2 mmol). The solution was stirred at 100° C. under N₂ for 12 h. The reaction mixture was quenched with ice water, extracted with EA (50 mL×3). The combined organic layer was dried over anhydrous Na₂SO₄, filtered, concentrated under reduced pressure. The residue was purified by flash chromatography (PE/EA=10:1) to give product as yellow solid (1 g, 69%). Mass (m/z): 603.3 [M+H]⁺.

Step 4. Preparation of (4-{3-[2,6-bis(benzyloxy)pyridin-3-yl]-1-methylindazol-6-yl}-3,6-dihydro-2H-pyridin-1-yl) tert-butyl formate: To a solution of (4-{3-[2,6-bis(benzyloxy)pyridin-3-yl]-1-methylindazol-6-yl}-3,6-dihydro-2H-pyridin-1-yl) tert-butyl formate (1 g, 1.7 mmol) in MeOH was added Pd/C (20 mg, 0.17 mmol) under N₂ atmosphere. The suspension was degassed and purged with H₂ 3 times. the reaction mixture was stirred at RT under H₂ for 24 h. The reaction mixture was filtered, concentrated under reduced pressure. The residue was purified by flash chromatography (PE/EA=1:1) to afford product as white solid (124 mg, 18%). Mass (m/z): 427.2 [M+H]⁺.

### Intermediate S: tert-butyl 4-(3-(2,4-dioxotetrahydropyrimidin-1 (2H)-yl) imidazo[1,2-a]pyridin-7-yl)piperidine-1-carboxylate

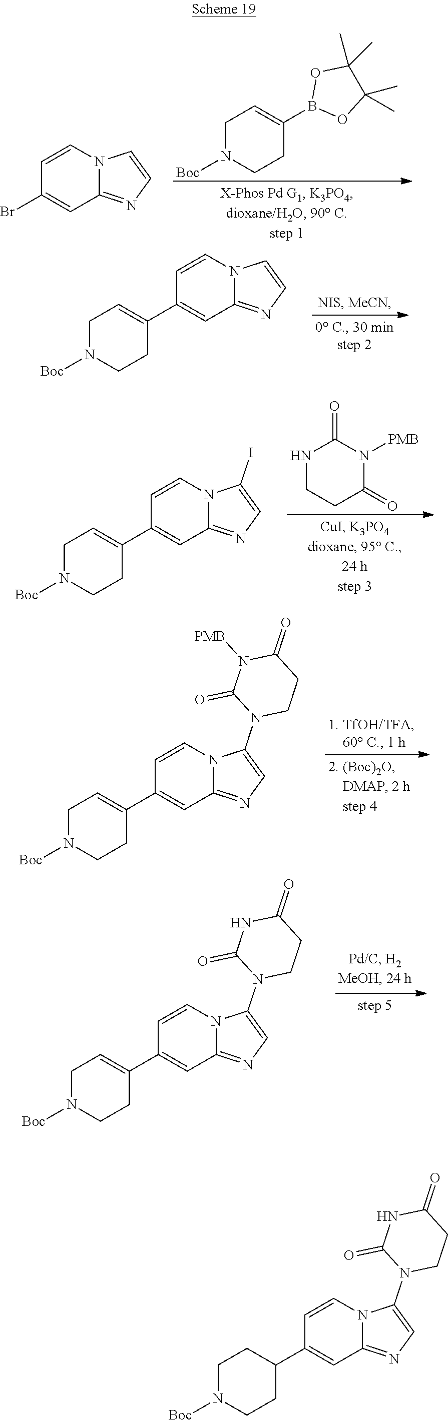

Step 1. Preparation of tert-butyl (4-{imidazo[1,2-a]pyridin-7-yl}-3,6-dihydro-2H-pyridin-1-yl) formate: To a solution of 7-bromoimidazo[1,2-a]pyridine (5 g, 25.4 mmol) in 10:1 dioxane/H₂O (50 mL) was added tert-butyl [4-(4,4,5,5-tetramethyl-1,3,2-dioxaborolan-2-yl)-3,6-dihydro-2H-pyridin-1-yl]formate (11.82 g, 38 mmol), K₃PO₄ (10.78 g, 50.8 mmol) and X phos Pd G1 (2.15 g, 2.5 mmol). The reaction was stirred at 90° C. under N₂ for 12 h. The reaction mixture was quenched with ice water, extracted with EA (80 mL×3). The combined organic layer was dried over with anhydrous Na₂SO₄, filtered, concentrated under reduced pressure. The residue was purified by flash chromatography (DCM/MeOH=10:1) to give the product as a yellow solid (7.2 g, 90%). Mass (m/z): 300.2 [M+H]⁺.

Step 2. Preparation of tert-butyl (4-{3-iodoimidazo[1,2-a]pyridin-7-yl}-3,6-dihydro-2H-pyridin-1-yl) formate: To a solution of tert-butyl (4-{imidazo[1,2-a]pyridin-7-yl}-3,6-dihydro-2H-pyridin-1-yl) formate (7 g, 23.3 mmol) in MeCN was added NIS (5.77 g, 25.6 mmol). The re action was stirred at room temperature for 30 min. The reaction mixture was filtered. The filter cake was dried under vacuum to afford product as yellow solid (9 g, 87%). Mass (m/z): 426.1 [M+H]⁺.

Step 3. Preparation of tert-butyl (4-{3-[3-(2-methoxy-5-methylphenyl)-2,4-dioxo-1,3-diazinan-1-yl]imidazo[1,2-a]pyridin-7-yl}-3,6-dihydro-2H-pyridin-1-yl) formate: To a solution of tert-butyl (4-{3-iodoimidazo[1,2-a]pyridin-7-yl}-3,6-dihydro-2H-pyridin-1-yl) formate (4.8 g, 17.5 mmol) in dioxane (50 mL) was added 3-(2-methoxy-5-methylphenyl)-1,3-diazinane-2,4-dione (3.18 g, 13.5 mmol), CuI (0.43 g, 2.3 mmol) and K₃PO₄ (4.80 g, 22.6 mmol). The reaction was stirred at 95° C. under N₂ for 24 h. The reaction mixture was quenched with ice water, extracted with EtOAc (50 mL×3). The combined organic layer was dried over anhydrous Na₂SO₄, filtered, concentrated under reduced pressure. The residue was purified by flash chromatography (PE/EA=1:1) to give the product as yellow solid (2.4 g, 38%). Mass (m/z): 532.3 [M+H]⁺.

Step 4. Preparation of tert-butyl {4-[3-(2,4-dioxo-1,3-diazinan-1-yl) imidazo[1,2-a]pyridin-7-yl]-3,6-dihydro-2H-pyridin-1-yl}formate: To tert-butyl (4-{3-[3-(2-methoxy-5-methylphenyl)-2,4-dioxo-1,3-diazinan-1-yl]imidazo[1,2-a]pyridin-7-yl}-3,6-dihydro-2H-pyridin-1-yl) formate (2 g, 3.8 mmol) was added 20% TfOH in TFA (10 mL) and the resulting mixture was stirred at 60° C. for 1 h. The reaction mixture was cooled to room temperature and concentrated in vacuo to remove TFA. The red residue was dissolved in water (15 ml), stirred at room temperature for 5 minutes. The mixture was filtered. The filter cake was washed by water (2×5 mL). The aqueous solution was then neutralized with solid NaHCO₃ to ˜pH 7. THF (20 mL) was added to the aqueous mixture followed by the addition of (Boc)₂O (1.66 g, 7.6 mol) and 4-DMAP (50 mg, 0.3 mmol). The reaction was stirred at room temperature for 1 h and then diluted with EtOAc (15 mL). The organic phases were separated, and the aqueous phase was extracted with EtOAc (2×30 ml). The combined organic phases were dried over anhydrous Na₂SO₄, filtered, and concentrated under reduced pressure. The residue was purified by flash chromatography (1% Et₃N/EtOAc) to afford product as off-white solid (500 mg, 32%). Mass (m/z): 412.1 [M+H]⁺.

Step 5. Preparation of tert-butyl {4-[3-(2,4-dioxo-1,3-diazinan-1-yl) imidazo[1,2-a]pyridin-7-yl]piperidin-1-yl}formate: To a solution of tert-butyl {4-[3-(2,4-dioxo-1,3-diazinan-1-yl) imidazo[1,2-a]pyridin-7-yl]-3,6-dihydro-2H-pyridin-1-yl}formate (160 mg, 0.39 mmol) in MeOH (4 mL) was added Pd/C (4 mg, 0.04 mmol) under N₂ atmosphere. The suspension was degassed and purged with H₂ 3 times. the reaction mixture was stirred at RT under H₂ for 24 hrs.

The reaction mixture was filtered and filtrate was concentrated to afford product as white solid (130 mg, 78%). Mass (m/z): 414.3 [M+H]⁺.

### Synthesis of Examples

### Example 1: 3-(4-((8-(4-(3-(4-chloro-3-ethyl-1H-pyrrolo[2,3-b]pyridin-5-yl)phenyl)-3-oxopiperazin-1-yl)-8-oxooctyl)oxy)-1-oxoisoindolin-2-yl)piperidine-2,6-dione

### Scheme 20

Step 1. (General Step C₁) Preparation of tert-butyl 8-hydroxyoctanoate: To a solution of 8-hydroxyoctanoic acid (3.5 g, 0.022 mol) in toluene (50 mL) was added 1,1-di-tert-butoxy-N, N-dimethylmethanamine (8.94 g, 0.044 mol). The reaction mixture was stirred at 80° C. for 16 hrs. The solution was concentrated and the residue was purified by flash column (PE/EA=10:1) to afford desired product (1.9 g, 29% yield) as a yellow oil. Mass (m/z): 239.2 [M+Na]⁺.

Step 2. (General Step D) Preparation of tert-butyl 8-(tosyloxy) octanoate: To a solution of tert-butyl 8-hydroxyoctanoate (1.9 g, 8.79 mmol), DMAP (11 mg, 0.088 mmol) and TEA (1.77 g, 17.58 mmol) in DCM (15 mL) was added TsCl (2 g, 10.54 mmol) at 0° C. The reaction mixture was stirred at rt for 2 hrs. Water (30 mL) was added and the mixture was extracted with DCM (20 mL×3). The combined organic layers were washed with brine (20 mL×2), dried over Na₂SO₄. Then by filtration, the filtrate was concentrated. The residue was purified by flash column (PE/EA=1:1) to give the desired product as a light yellow oil (1.5 g, yield: 37%). Mass (m/z): 393.0 [M+Na]⁺.

Step 3. (General Step E) Preparation of tert-butyl 8-((2-(2,6-dioxopiperidin-3-yl)-1-oxoisoindolin-4-yl)oxy) octanoate: A mixture of tert-butyl 8-(tosyloxy) octanoate (200 mg, 0.54 mmol), 3-(4-hydroxy-1-oxoisoindolin-2-yl)piperidine-2,6-dione (140 mg, 0.54 mmol) and K₂CO₃ (112 mg, 0.81 mmol) in DMF (15 mL) was stirred at 50° C. under N₂ for 16 hrs. The reaction was filtered and concentrated. The crude product was purified by Prep-HPLC (CAN-H₂O 0.1% FA) to afford desired product (100 mg, 9% yield) as a light-yellow solid. Mass (m/z): 481.0 [M+Na]⁺.

Step 4. (General Step B2) Preparation of 8-((2-(2,6-dioxopiperidin-3-yl)-1-oxoisoindolin-4-yl)oxy) octanoic acid: To a mixture of tert-butyl 8-((2-(2,6-dioxopiperidin-3-yl)-1-oxoisoindolin-4-yl)oxy) octanoate (100 mg, 0.22 mmol) in DCM (20 mL) was added TFA (4 mL). The reaction mixture was stirred at rt for 2 hrs. The reaction mixture was concentrated under reduced pressure and dried overnight in a lyophilizer to afford desired product (90 mg, 84% yield). Mass (m/z): 403.0 [M+H]⁺.

Step 5. (General Step F) Preparation of 3-(4-((8-(4-(3-(4-chloro-3-ethyl-1H-pyrrolo[2,3-b]pyridin-5-yl)phenyl)-3-oxopiperazin-1-yl)-8-oxooctyl)oxy)-1-oxoisoindolin-2-yl)piperidine-2,6-dione: To a mixture of compound 8-((2-(2,6-dioxopiperidin-3-yl)-1-oxoisoindolin-4-yl)oxy) octanoic acid (45 mg, 0.11 mmol), 1-(3-(4-chloro-3-ethyl-1H-pyrrolo[2,3-b]pyridin-5-yl)phenyl)piperazin-2-one (42 mg, 0.11 mmol) and DIEA (42 mg, 0.33 mmol) in DMF (10 mL) under N₂ was added T3P (140 mg, 0.22 mmol, 50% in EA). The reaction mixture was stirred at room temperature for 16 hrs. Water (20 mL) was added and the mixture was extracted with DCM (20 mL×3). The combined organic layers were washed with brine (20 mL×3), dried over Na₂SO₄. Then by filtration, the filtrate was concentrated. The reaction is purified by prep-HPLC (ACN-H₂O, 0.1% FA) to afford desired product (15 mg, 16% yield) as a light-yellow solid. Mass (m/z): 739.0 [M+H]⁺. ¹H NMR (400 MHZ, DMSO-d₆) δ 11.81 (s, 1H), 10.98 (s, 1H), 8.12 (s, 1H), 7.47-7.24 (m, 7H), 5.09 (s, 1H), 4.23-4.10 (m, 7H), 3.84 (s, 3H), 2.91 (s, 3H), 2.38 (s, 2H), 1.98-1.27 (m, 7H).

### Example 2: 3-(4-((8-(4-(3-(4-chloro-3-ethyl-1H-pyrrolo[2,3-b]pyridin-5-yl)phenyl)-3-oxopiperazin-1-yl)-8-oxooctyl)oxy)-1-oxoisoindolin-2-yl)-1-methylpiperidine-2,6-dione

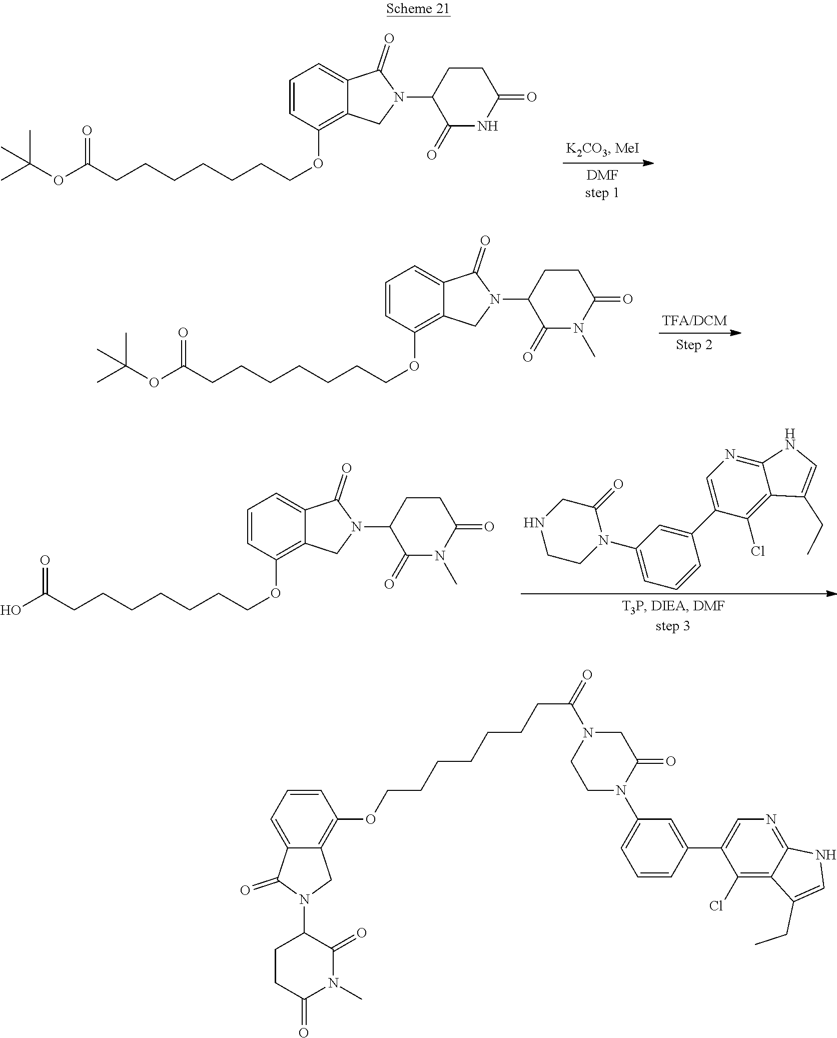 [SMILES: CC(C)(C)OC(=O)CCCCCCCOc1cccc2c1CN(C1CCC(=O)NC1=O)C2=O ; CN1C(=O)CCC(N2Cc3c(OCCCCCCCC(=O)OC(C)(C)C)cccc3C2=O)C1=O ; CN1C(=O)CCC(N2Cc3c(OCCCCCCCC(=O)O)cccc3C2=O)C1=O ; CCc1c[nH]c2ncc(-c3cccc(N4CCNCC4=O)c3)c(Cl)c12 ; CCc1c[nH]c2ncc(-c3cccc(N4CCN(C(=O)CCCCCCCOc5cccc6c5CN(C5CCC(=O)N(C)C5=O)C6=O)CC4=O)c3)c(Cl)c12]

Step 1. Preparation of tert-butyl 8-((2-(1-methyl-2,6-dioxopiperidin-3-yl)-1-oxoisoindolin-4-yl)oxy) octanoate: A mixture of compound tert-butyl 8-((2-(2,6-dioxopiperidin-3-yl)-1-oxoisoindolin-4-yl)oxy) octanoate (130 mg, 0.28 mmol), CH₃I (43 mg, 0.31 mmol) and K₂CO₃ (78 mg, 0.56 mmol) in DMF (10 mL) under N₂ was stirred at rt for 16 hrs. Water (30 mL) was added and the mixture was extracted with EA (20 mL×3). The combined organic layers were washed with brine (20 mL×3), dried over Na₂SO₄. Then by filtration, the filtrate was concentrated to afford compound 2 (120 mg, 89% yield) as a light-yellow solid. Mass (m/z): 496.0 [M+Na]⁺.

Step 2. Preparation of 8-((2-(1-methyl-2,6-dioxopiperidin-3-yl)-1-oxoisoindolin-4-yl)oxy) octanoic acid: Following General Step B2, 8-((2-(1-methyl-2,6-dioxopiperidin-3-yl)-1-oxoisoindolin-4-yl)oxy) octanoic acid was prepared as a yellow solid (100 mg, 94% yield). Mass (m/z): 417.0 [M+H]⁺.

Step 3. Preparation of 3-(4-((8-(4-(3-(4-chloro-3-ethyl-1H-pyrrolo[2,3-b]pyridin-5-yl)phenyl)-3-oxopiperazin-1-yl)-8-oxooctyl)oxy)-1-oxoisoindolin-2-yl)-1-methylpiperidine-2,6-dione: Following General Step F, 3-(4-((8-(4-(3-(4-chloro-3-ethyl-1H-pyrrolo[2,3-b]pyridin-5-yl)phenyl)-3-oxopiperazin-1-yl)-8-oxooctyl)oxy)-1-oxoisoindolin-2-yl)-1-methylpiperidine-2,6-dione was prepared as a light yellow solid (25 mg, 19% yield). Mass (m/z): 752.9 [M+H]⁺. ¹H NMR (400 MHZ, DMSO-d₆) δ 11.80 (s, 1H), 8.12-8.11 (m, 1H), 7.52-7.21 (m, 8H), 5.16 (dd, J=12 Hz, 4 Hz, 1H), 4.37-4.08 (m, 6H), 3.83 (s, 2H), 3.77 (d, J=8 Hz, 2H), 2.98 (s, 3H), 2.94-2.88 (m, 2H), 2.74-2.66 (m, 2H), 2.39-2.32 (m, 2H), 1.99-1.96 (m, 1H), 1.72-1.71 (m, 2H), 1.52-1.22 (m, 12H).

### Example 3: 3-ethyl-1H-pyrrolo 3-(4-(2-(4-(3-(4-chloro-3-ethyl-1H-pyrrolo[2,3-b]pyridin-5-yl)phenyl)-3-oxopiperazin-1-yl)-2-oxoethoxy)-1-oxoisoindolin-2-yl)piperidine-2,6-dione

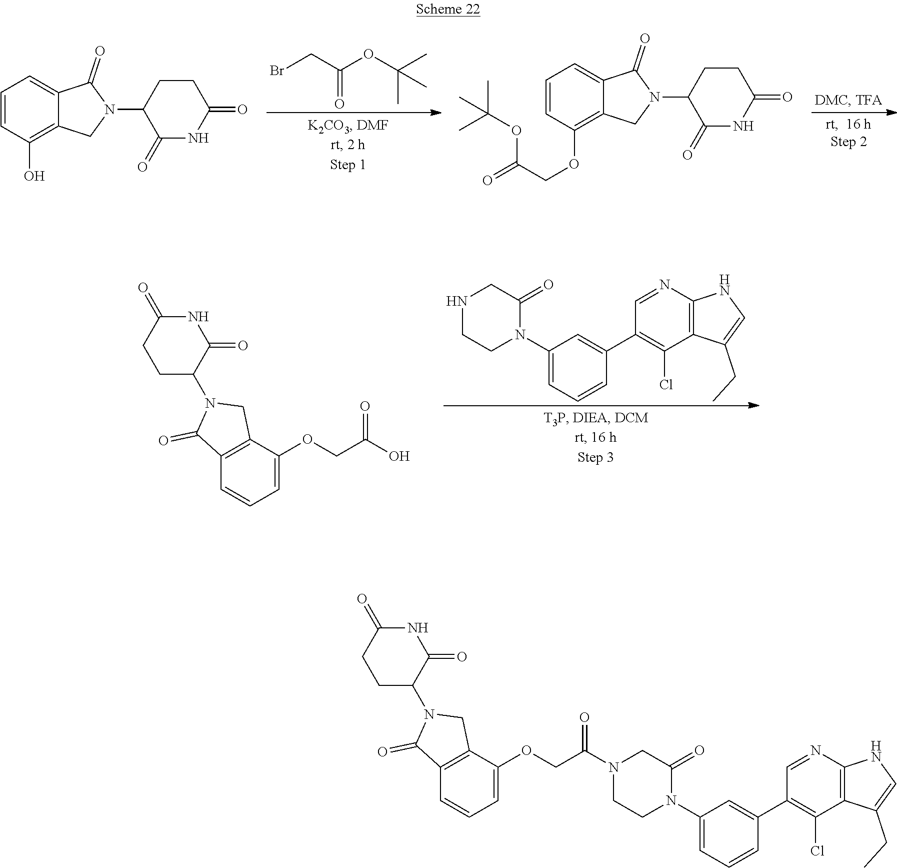

Step 1. Preparation of 3-(4-{[2-(tert-butoxy) prop-2-en-1-yl]oxy}-1-oxo-3H-isoindol-2-yl)piperidine-2,6-dione: Following General Step E, 3-(4-{[2-(tert-butoxy) prop-2-en-1-yl]oxy}-1-oxo-3H-isoindol-2-yl)piperidine-2,6-dione was prepared as a white solid (610 mg, 38%). Mass (m/z): 375.0 [M+H]⁺.

Step 2. Preparation of {[2-(2,6-dioxopiperidin-3-yl)-1-oxo-3H-isoindol-4-yl]oxy}acetic acid: Following General Step B2, {[2-(2,6-dioxopiperidin-3-yl)-1-oxo-3H-isoindol-4-yl]oxy}acetic acid was prepared as a white solid (410 mg, 72%). Mass (m/z): 319.0 [M+H]⁺.

Step 3. Preparation of 3-(4-{2-[4-(3-{4-chloro-3-ethyl-1H-pyrrolo[2,3-b]pyridin-3-yl}phenyl)-3-oxopiperazin-1-yl]-2-oxoethoxy}-1-oxo-3H-isoindol-2-yl)piperidine-2,6-dione: Following General Step F, 3-(4-{2-[4-(3-{4-chloro-3-ethyl-1H-pyrrolo[2,3-b]pyridin-3-yl}phenyl)-3-oxopiperazin-1-yl]-2-oxoethoxy}-1-oxo-3H-isoindol-2-yl)piperidine-2,6-dione was prepared as a white solid (18 mg, 19%). Mass (m/z): 654.8 [M+H]⁺.

### Example 4: 3-(4-(4-(4-(3-(4-chloro-3-ethyl-1H-pyrrolo[2,3-b]pyridin-5-yl)phenyl)-3-oxopiperazin-1-yl)-4-oxobutoxy)-1-oxoisoindolin-2-yl)piperidine-2,6-dione

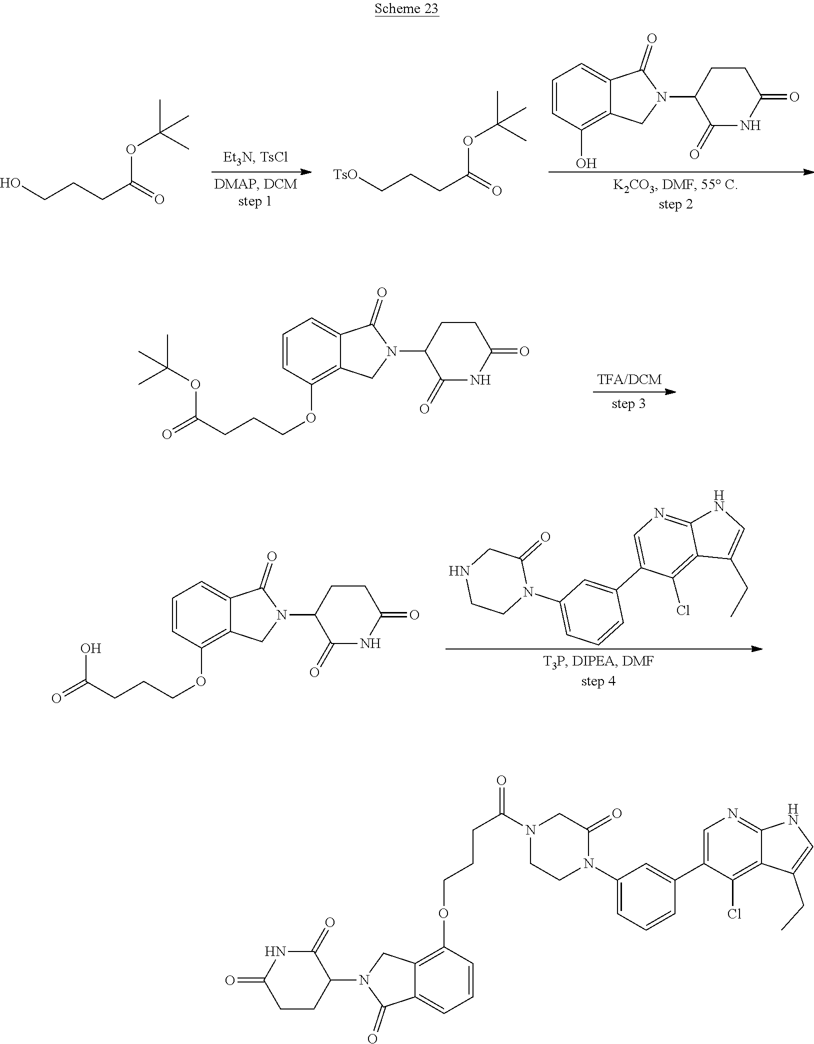

Step 1. Preparation of tert-butyl 4-(tosyloxy) butanoate: Following General Step D, tert-butyl 4-(tosyloxy) butanoate was prepared as a light yellow oil (600 mg, yield: 61%). Mass (m/z): 336.9 [M+H]⁺.

Step 2. Preparation of tert-butyl 4-((2-(2,6-dioxopiperidin-3-yl)-1-oxoisoindolin-4-yl)oxy) butanoate: Following General Step E, tert-butyl 4-((2-(2,6-dioxopiperidin-3-yl)-1-oxoisoindolin-4-yl)oxy) butanoate was prepared as a light yellow solid (150 mg, 19% yield). Mass (m/z): 424.8 [M+Na]⁺.

Step 3. Preparation of 4-((2-(2,6-dioxopiperidin-3-yl)-1-oxoisoindolin-4-yl)oxy) butanoic acid: Following General Step B2, 4-((2-(2,6-dioxopiperidin-3-yl)-1-oxoisoindolin-4-yl)oxy) butanoic acid was prepared as a yellow solid (110 mg, 85% yield). Mass (m/z): 346.9 [M+H]⁺.

Step 4. Preparation of 3-(4-(4-(4-(3-(4-chloro-3-ethyl-1H-pyrrolo[2,3-b]pyridin-5-yl)phenyl)-3-oxopiperazin-1-yl)-4-oxobutoxy)-1-oxoisoindolin-2-yl)piperidine-2,6-dione: Following General Step F, 3-(4-(4-(4-(3-(4-chloro-3-ethyl-1H-pyrrolo[2,3-b]pyridin-5-yl)phenyl)-3-oxopiperazin-1-yl)-4-oxobutoxy)-1-oxoisoindolin-2-yl)piperidine-2,6-dione was prepared as a light yellow solid (50 mg, 33% yield). Mass (m/z): 682.7 [M+H]⁺.

### Example 5: 3-(4-((6-(4-(3-(4-chloro-3-ethyl-1H-pyrrolo[2,3-b]pyridin-5-yl)phenyl)-3-oxopiperazin-1-yl)-6-oxohexyl)oxy)-1-oxoisoindolin-2-yl)piperidine-2,6-dione

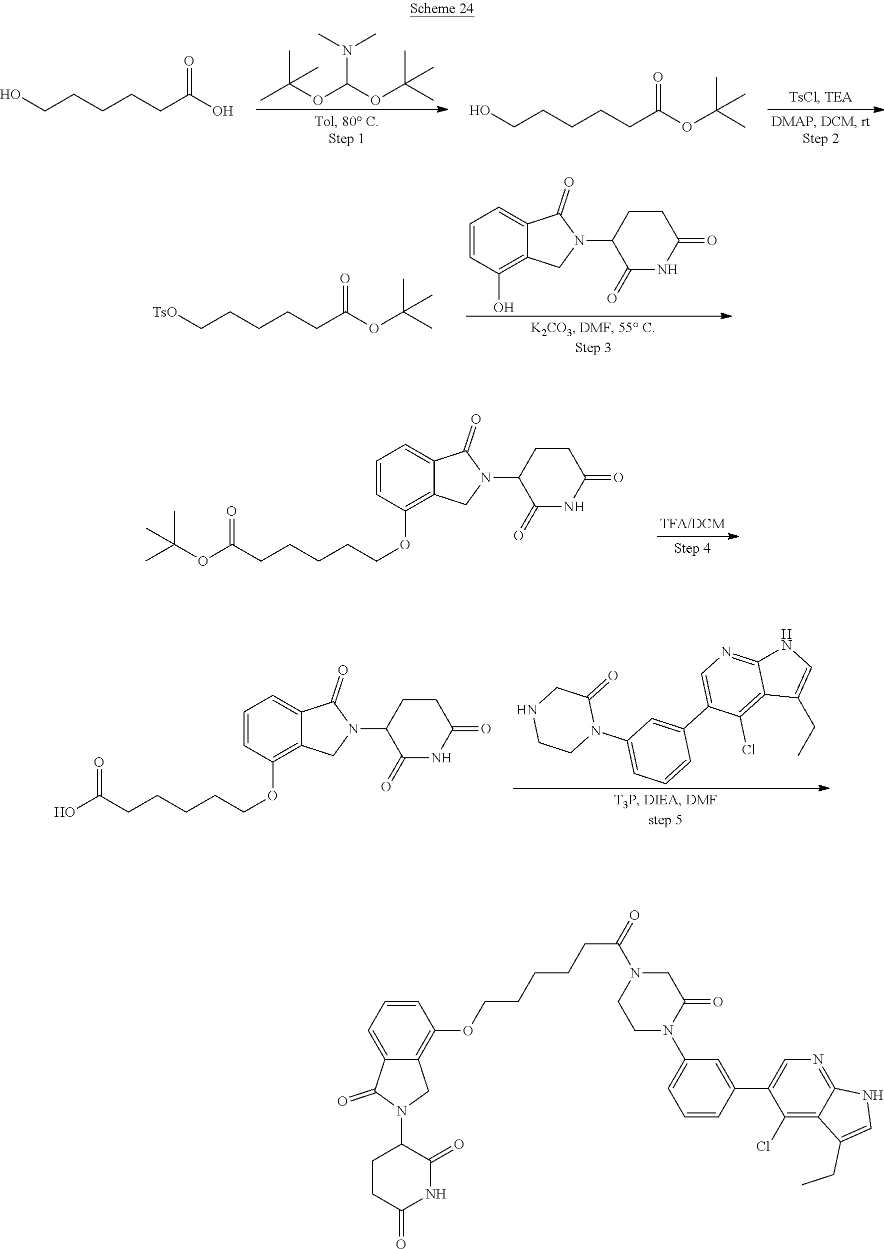

Step 1. Preparation of tert-butyl 6-hydroxyhexanoate: Following General Step C₁, tert-butyl 6-hydroxyhexanoate was prepared as a pale-yellow oil (1.1 g, 25% yield). Mass (m/z): 211.0 [M+Na]⁺.

Step 2. Preparation of tert-butyl 6-(tosyloxy) hexanoate: Following General Step D, tert-butyl 6-(tosyloxy) hexanoate was prepared as a light yellow oil (700 mg, yield: 35%). Mass (m/z): 365.0 [M+Na]⁺.

Step 3. Preparation tert-butyl 6-((2-(2,6-dioxopiperidin-3-yl)-1-oxoisoindolin-4-yl)oxy) hexanoate: Following General Step E, tert-butyl 6-((2-(2,6-dioxopiperidin-3-yl)-1-oxoisoindolin-4-yl)oxy) hexanoate was prepared as a light yellow solid (90 mg, 23% yield). Mass (m/z): 453.0 [M+Na]⁺.

Step 4. Preparation of 6-((2-(2,6-dioxopiperidin-3-yl)-1-oxoisoindolin-4-yl)oxy) hexanoic acid: Following General Step B2, 6-((2-(2,6-dioxopiperidin-3-yl)-1-oxoisoindolin-4-yl)oxy) hexanoic acid was prepared as a light yellow oil (70 mg, 64% yield). Mass (m/z): 375.0 [M+H]⁺.

Step 5. Preparation of 3-(4-((6-(4-(3-(4-chloro-3-ethyl-1H-pyrrolo[2,3-b]pyridin-5-yl)phenyl)-3-oxopiperazin-1-yl)-6-oxohexyl)oxy)-1-oxoisoindolin-2-yl)piperidine-2,6-dione: Following General Step F, 3-(4-((6-(4-(3-(4-chloro-3-ethyl-1H-pyrrolo[2,3-b]pyridin-5-yl)phenyl)-3-oxopiperazin-1-yl)-6-oxohexyl)oxy)-1-oxoisoindolin-2-yl)piperidine-2,6-dione was prepared as a light yellow solid (12 mg, 24% yield). Mass (m/z): 710.9 [M+H]⁺.

### Example 6: 3-(4-((7-(4-(3-(4-chloro-3-ethyl-1H-pyrrolo[2,3-b]pyridin-5-yl)phenyl)-3-oxopiperazin-1-yl)-7-oxoheptyl)oxy)-1-oxoisoindolin-2-yl)piperidine-2,6-dione

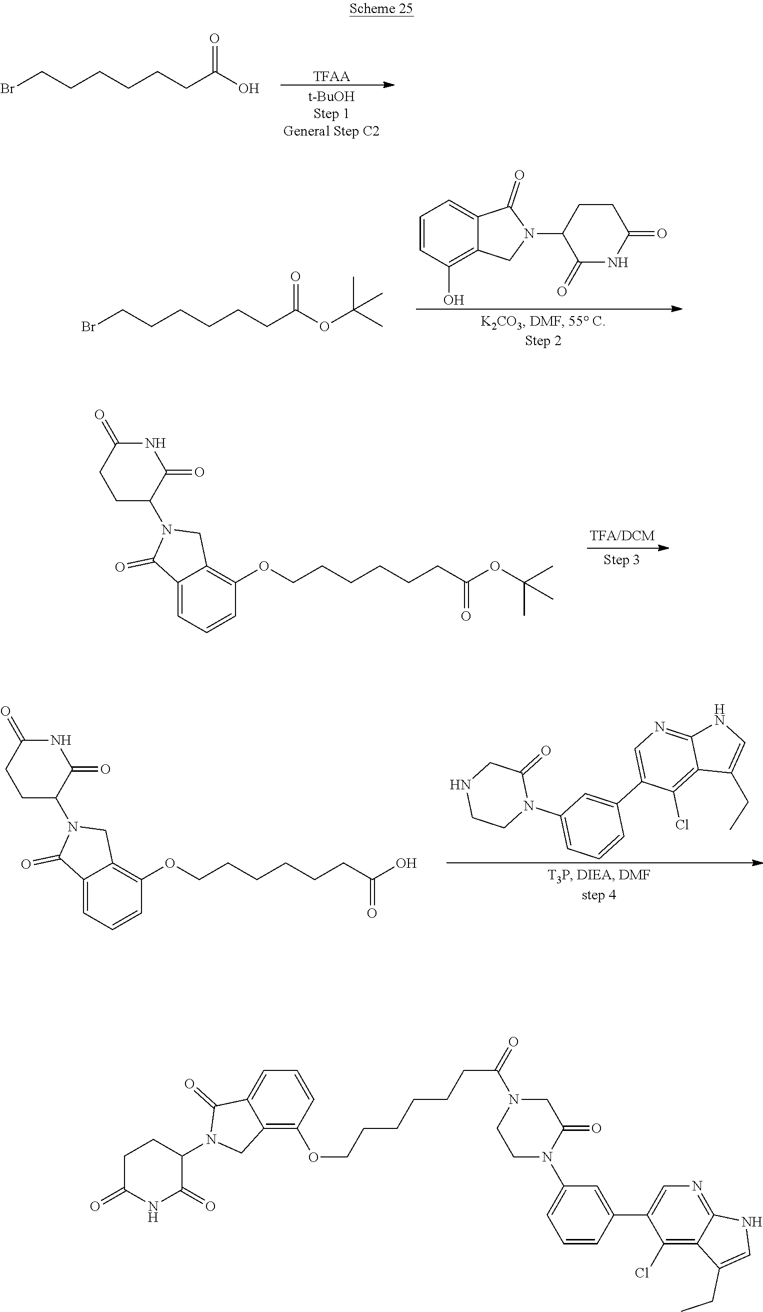

Step 1. (General Step C₂) Preparation of tert-butyl 7-bromoheptanoate: To a solution of 7-bromoheptanoic acid (2 g, 9.56 mmol) in DCM (20 mL) at 0° C. was added TFAA (4.6 g, 22.00 mmol). The reaction mixture was stirred at room temperature for 2 hrs. Then tert-butyl alcohol (2.52 g, 34.43 mmol) was added to the reaction. The reaction mixture was stirred at rt for 16 hrs. Water (50 mL) was added and the mixture was extracted with EA (20 mL×3). The combined organic layers were washed with brine (20 mL×2), dried over Na₂SO₄. Then by filtration, the filtrate was concentrated. The crude product was purified by silica gel column chromatography (PE:EA=20:1) to give the product tert-butyl 7-bromoheptanoate as a yellow oil (2 g, 70%).

Step 2. Preparation of tert-butyl 7-((2-(2,6-dioxopiperidin-3-yl)-1-oxoisoindolin-4-yl)oxy) heptanoate: To a solution of tert-butyl 7-bromoheptanoate (509 mg, 1.92 mmol) and 3-(4-hydroxy-1-oxo-3H-isoindol-2-yl)piperidine-2,6-dione (500 mg, 1.92 mmol) in DMF (5 mL) was added potassium carbonate (398 mg, 2.88 mmol). The reaction mixture was stirred at 55° C. for 16 hrs. Water (100 mL) was added and the reaction mixture was extracted with EA (50 mL×3). The combined organic layers were washed with brine (50 mL×3), dried over Na₂SO₄. Then by filtration, the filtrate was concentrated. The crude product was The organic phase was concentrated and purified by Pre-HPLC [chromatographic column:—Gemini-C₁₈ 150×21.2 mm, 5 um, mobile phase: ACN-H₂O (0.1% FA), gradient: 45-70] to give the product tert-butyl 7-((2-(2,6-dioxopiperidin-3-yl)-1-oxoisoindolin-4-yl)oxy) heptanoate as a brown solid (200 mg, 22%). Mass (m/z): 466.9 [M+Na]⁺.

Step 3. Preparation of 7-((2-(2,6-dioxopiperidin-3-yl)-1-oxoisoindolin-4-yl)oxy) heptanoic acid: Following General Step B2, 7-((2-(2,6-dioxopiperidin-3-yl)-1-oxoisoindolin-4-yl)oxy) heptanoic acid was prepared as a yellow oil (200 mg, 91%). Mass (m/z): 388.9 [M+H]⁺.

Step 4. Preparation of 3-(4-((7-(4-(3-(4-chloro-3-ethyl-1H-pyrrolo[2,3-b]pyridin-5-yl)phenyl)-3-oxopiperazin-1-yl)-7-oxoheptyl)oxy)-1-oxoisoindolin-2-yl)piperidine-2,6-dione: Following General Step F, 3-(4-((7-(4-(3-(4-chloro-3-ethyl-1H-pyrrolo[2,3-b]pyridin-5-yl)phenyl)-3-oxopiperazin-1-yl)-7-oxoheptyl)oxy)-1-oxoisoindolin-2-yl)piperidine-2,6-dione was prepared as a white solid (9 mg, 3.5%). Mass (m/z): 724.7 [M+H]⁺.

### Example 7: 3-(4-((10-(4-(3-(4-chloro-3-ethyl-1H-pyrrolo[2,3-b]pyridin-5-yl)phenyl)-3-oxopiperazin-1-yl)-10-oxodecyl)oxy)-1-oxoisoindolin-2-yl)piperidine-2,6-dione

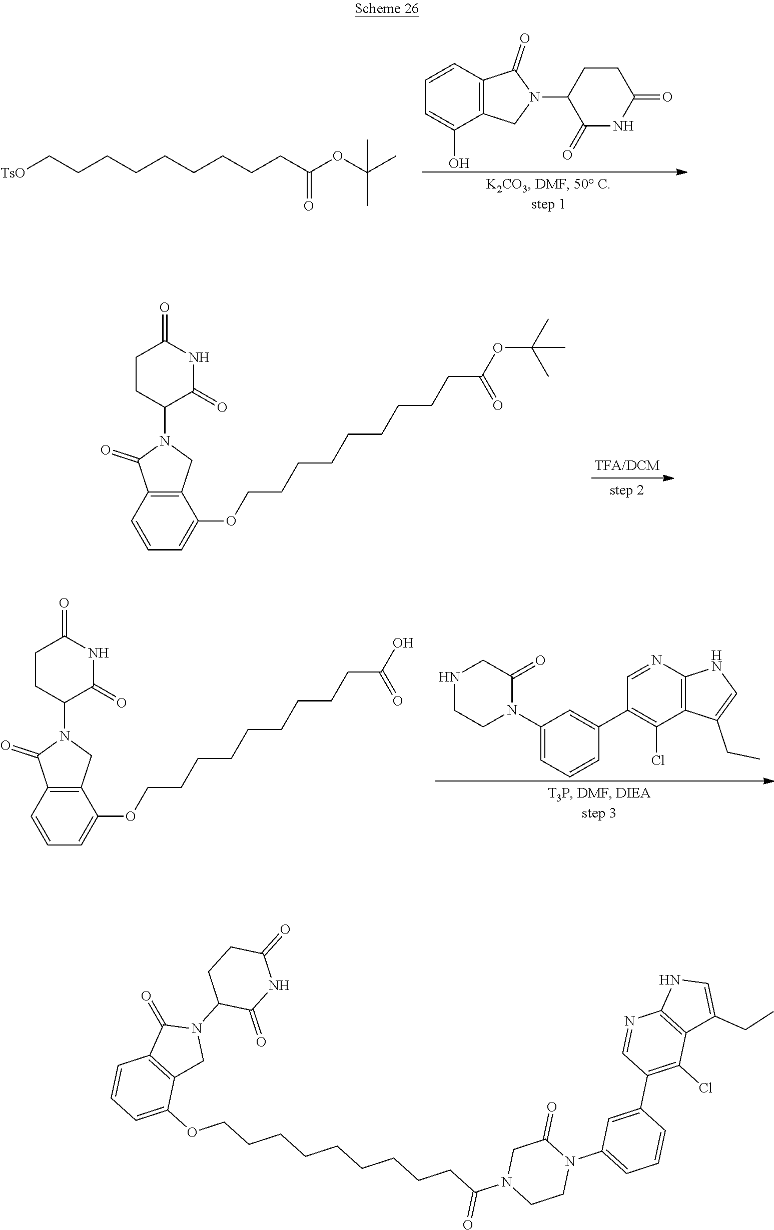 [SMILES: Cc1ccc(S(=O)(=O)OCCCCCCCCCC(=O)OC(C)(C)C)cc1 ; O=C1CCC(N2Cc3c(O)cccc3C2=O)C(=O)N1 ; CC(C)(C)OC(=O)CCCCCCCCCOc1cccc2c1CN(C1CCC(=O)NC1=O)C2=O ; O=C(O)CCCCCCCCCOc1cccc2c1CN(C1CCC(=O)NC1=O)C2=O ; CCc1c[nH]c2ncc(-c3cccc(N4CCNCC4=O)c3)c(Cl)c12 ; CCc1c[nH]c2ncc(-c3cccc(N4CCN(C(=O)CCCCCCCCCOc5cccc6c5CN(C5CCC(=O)NC5=O)C6=O)CC4=O)c3)c(Cl)c12]

Step 1. Preparation of tert-butyl 10-((2-(2,6-dioxopiperidin-3-yl)-1-oxoisoindolin-4-yl)oxy) decanoate: Following General Step E, tert-butyl 10-((2-(2,6-dioxopiperidin-3-yl)-1-oxoisoindolin-4-yl)oxy) decanoate was prepared as a light yellow solid (400 mg, 54% yield). Mass (m/z): 508.8 [M+Na]⁺.

Step 2. Preparation of 10-((2-(2,6-dioxopiperidin-3-yl)-1-oxoisoindolin-4-yl)oxy) decanoic acid: Following General Step B2, 10-((2-(2,6-dioxopiperidin-3-yl)-1-oxoisoindolin-4-yl)oxy) decanoic acid was prepared as a light yellow oil (300 mg, 84% yield). Mass (m/z): 431.0 [M+H]⁺.

Step 3. Preparation of 3-(4-((10-(4-(3-(4-chloro-3-ethyl-1H-pyrrolo[2,3-b]pyridin-5-yl)phenyl)-3-oxopiperazin-1-yl)-10-oxodecyl)oxy)-1-oxoisoindolin-2-yl)piperidine-2,6-dione: Following General Step F, 3-(4-((10-(4-(3-(4-chloro-3-ethyl-1H-pyrrolo[2,3-b]pyridin-5-yl)phenyl)-3-oxopiperazin-1-yl)-10-oxodecyl)oxy)-1-oxoisoindolin-2-yl)piperidine-2,6-dione was prepared as a light yellow solid (18 mg, 9% yield). Mass (m/z): 767.0 [M+H]⁺.

### Example 8: 3-ethyl-1H-pyrrolo 3-(4-((8-(4-(3-(2-chloro-3-ethyl-1H-pyrrolo[2,3-b]pyridin-5-yl)phenyl)-3-oxopiperazin-1-yl)-8-oxooctyl)oxy)-1-oxoisoindolin-2-yl)piperidine-2,6-dione

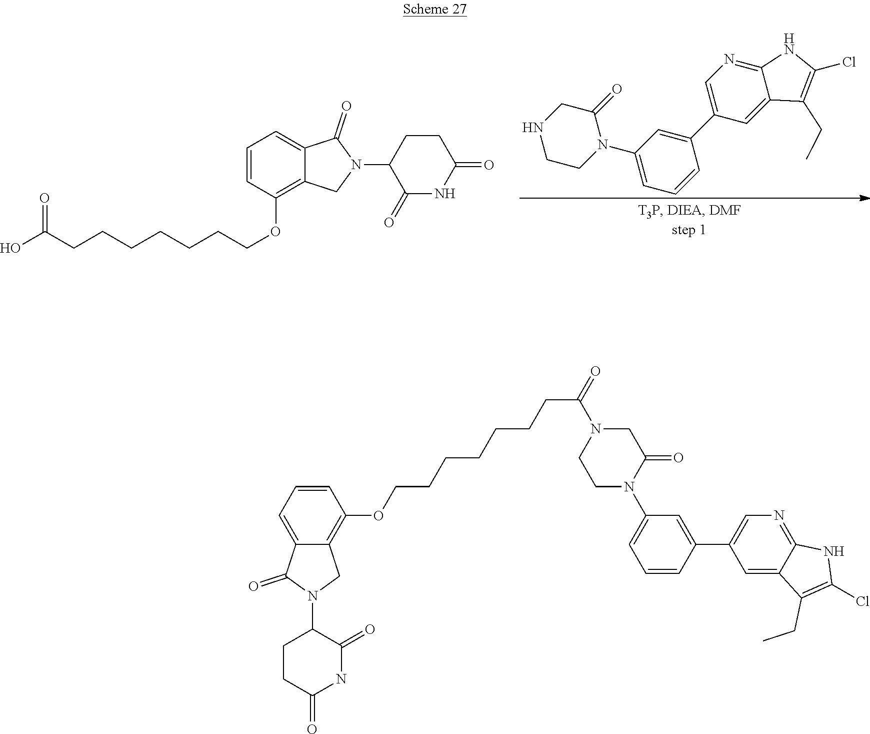

Step 1. Preparation of 3-[4-({8-[4-(3-{6-chloro-3-ethyl-1H-pyrrolo[2,3-b]pyridin-3-yl}phenyl)-3-oxopiperazin-1-yl]-8-oxooctyl}oxy)-1-oxo-3H-isoindol-2-yl]piperidine-2,6-dione: Following General Step F, 3-[4-({8-[4-(3-{6-chloro-3-ethyl-1H-pyrrolo[2,3-b]pyridin-3-yl}phenyl)-3-oxopiperazin-1-yl]-8-oxooctyl}oxy)-1-oxo-3H-isoindol-2-yl]piperidine-2,6-dione was prepared as a light yellow solid (70 mg, 99% yield). Mass (m/z): 739.0 [M+H]⁺.

### Example 9: 4-((8-(4-(3-(4-chloro-3-ethyl-1H-pyrrolo[2,3-b]pyridin-5-yl)phenyl)-3-oxopiperazin-1-yl)-8-oxooctyl)oxy)-2-(2,6-dioxopiperidin-3-yl)isoindoline-1,3-dione

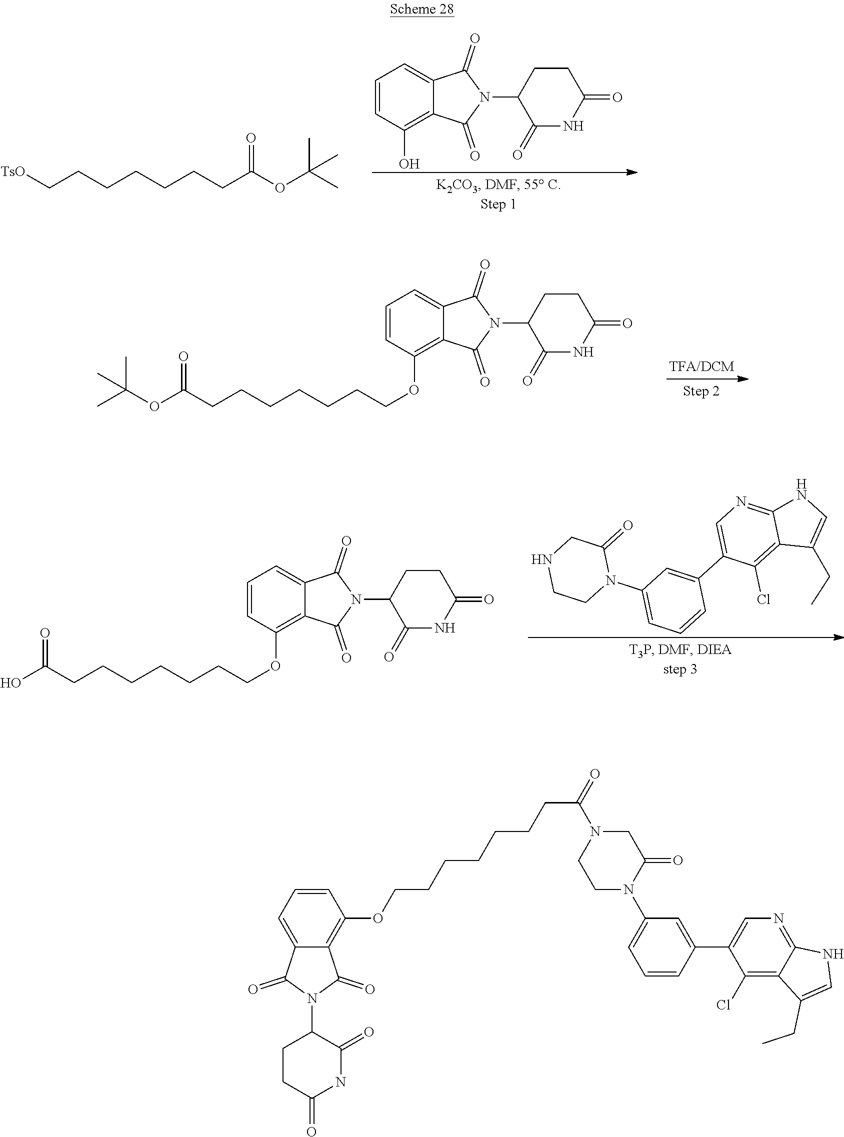

Step 1. Preparation of tert-butyl 8-((2-(2,6-dioxopiperidin-3-yl)-1,3-dioxoisoindolin-4-yl)oxy) octanoate: Following General Step E, tert-butyl 8-((2-(2,6-dioxopiperidin-3-yl)-1,3-dioxoisoindolin-4-yl)oxy) octanoate was prepared as a light yellow solid (90 mg, 11% yield). Mass (m/z): 494.8 [M+Na]⁺.

Step 2. Preparation of 8-((2-(2,6-dioxopiperidin-3-yl)-1,3-dioxoisoindolin-4-yl)oxy) octanoic acid: Following General Step B2, 8-((2-(2,6-dioxopiperidin-3-yl)-1,3-dioxoisoindolin-4-yl)oxy) octanoic acid was prepared as a light yellow oil (70 mg, 88% yield). Mass (m/z): 417.0 [M+H]⁺.

Step 3. Preparation of 4-((8-(4-(3-(4-chloro-3-ethyl-1H-pyrrolo[2,3-b]pyridin-5-yl)phenyl)-3-oxopiperazin-1-yl)-8-oxooctyl)oxy)-2-(2,6-dioxopiperidin-3-yl)isoindoline-1,3-dione: Following General Step F, 4-((8-(4-(3-(4-chloro-3-ethyl-1H-pyrrolo[2,3-b]pyridin-5-yl)phenyl)-3-oxopiperazin-1-yl)-8-oxooctyl)oxy)-2-(2,6-dioxopiperidin-3-yl)isoindoline-1,3-dione was prepared as a light yellow solid (20 mg, 15% yield). Mass (m/z): 752.9 [M+H]⁺.

### Example 10: 3-(4-((8-(4-(3-(4-chloro-3-ethyl-1H-pyrrolo[2,3-b]pyridin-5-yl)phenyl)-3-oxopiperazin-1-yl)-8-oxooctyl)amino)-1-oxoisoindolin-2-yl)piperidine-2,6-dione

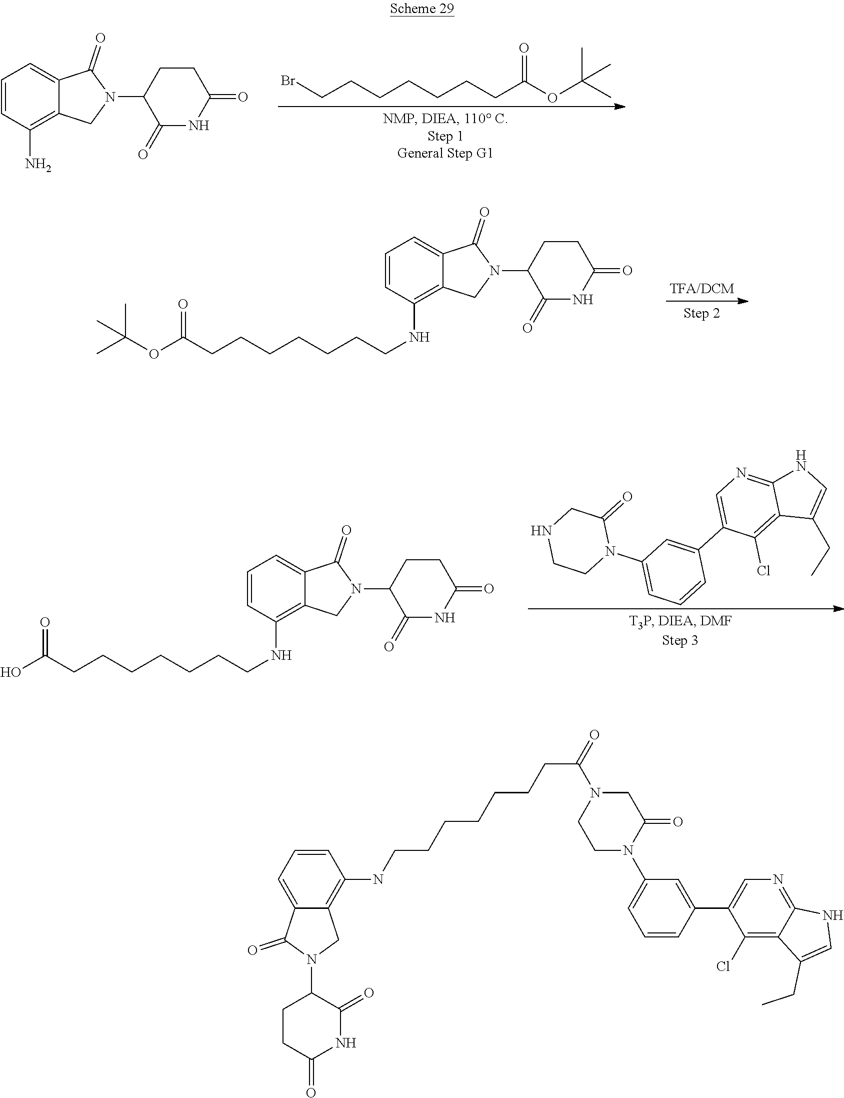 [SMILES: Nc1cccc2c1CN(C1CCC(=O)NC1=O)C2=O ; CC(C)(C)OC(=O)CCCCCCCBr ; CC(C)(C)OC(=O)CCCCCCCNc1cccc2c1CN(C1CCC(=O)NC1=O)C2=O ; O=C(O)CCCCCCCNc1cccc2c1CN(C1CCC(=O)NC1=O)C2=O ; CCc1c[nH]c2ncc(-c3cccc(N4CCNCC4=O)c3)c(Cl)c12 ; CCc1c[nH]c2ncc(-c3cccc(N4CCN(C(=O)CCCCCCCNc5cccc6c5CN(C5CCC(=O)NC5=O)C6=O)CC4=O)c3)c(Cl)c12]

Step 1. (General Step G1) Preparation of tert-butyl 8-((2-(2,6-dioxopiperidin-3-yl)-1-oxoisoindolin-4-yl)amino) octanoate: To a solution of tert-butyl 8-bromooctanoate (600 mg, 2.14 mmol) and 3-(4-hydroxy-1-oxo-3H-isoindol-2-yl)piperidine-2,6-dione (557 mg, 2.14 mmol) in NMP (10 mL) was added DIEA (833 mg, 6.44 mmol). The reaction mixture was stirred at 110° C. for 16 hrs. Water (100 mL) was added and the mixture was extracted with EA (50 mL×3). The combined organic layers were washed with brine (50 mL×2), dried over Na₂SO₄. Then by filtration, the filtrate was concentrated. The crude product was purified by Pre-HPLC [chromatographic column:—Gemini-C₁₈ 150×21.2 mm, 5 um, mobile phase: ACN-H₂O (0.1% FA), gradient: 50-80] to give the product as a white solid (200 mg, 19%). Mass (m/z): 480.0 [M+Na]⁺.

Step 2. Preparation of 8-((2-(2,6-dioxopiperidin-3-yl)-1-oxoisoindolin-4-yl)amino) octanoic acid: Following General Step B2, 8-((2-(2,6-dioxopiperidin-3-yl)-1-oxoisoindolin-4-yl)amino) octanoic acid was prepared as a yellow oil (200 mg, 91%). Mass (m/z): 401.9 [M+H]⁺.

Step 3. Preparation of 3-(4-((8-(4-(3-(4-chloro-3-ethyl-1H-pyrrolo[2,3-b]pyridin-5-yl)phenyl)-3-oxopiperazin-1-yl)-8-oxooctyl)amino)-1-oxoisoindolin-2-yl)piperidine-2,6-dione: Following General Step F, 3-(4-((8-(4-(3-(4-chloro-3-ethyl-1H-pyrrolo[2,3-b]pyridin-5-yl)phenyl)-3-oxopiperazin-1-yl)-8-oxooctyl)amino)-1-oxoisoindolin-2-yl)piperidine-2,6-dione was prepared as a white solid (42 mg, 10%). Mass (m/z): 737.8 [M+H]⁺.

### Example 11: 3-(4-((8-(4-(3-(4-chloro-3-ethyl-1H-pyrrolo[2,3-b]pyridin-5-yl)phenyl)-3-oxopiperazin-1-yl)-8-oxooctyl)(methyl)amino)-1-oxoisoindolin-2-yl)piperidine-2,6-dione

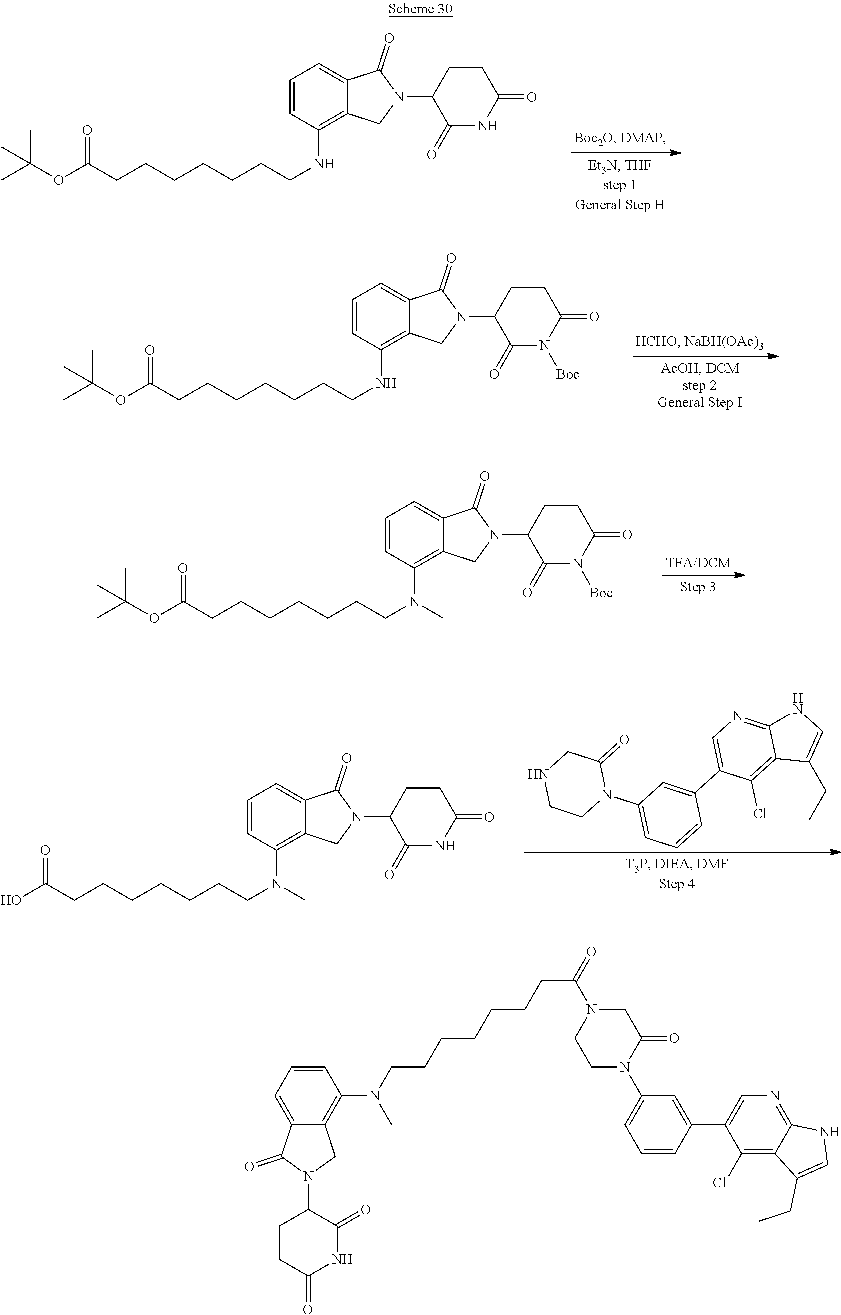 [SMILES: CC(C)(C)OC(=O)CCCCCCCNc1cccc2c1CN(C1CCC(=O)NC1=O)C2=O ; CC(C)(C)OC(=O)CCCCCCCNc1cccc2c1CN(C1CCC(=O)N(C(=O)OC(C)(C)C)C1=O)C2=O ; CN(CCCCCCCC(=O)OC(C)(C)C)c1cccc2c1CN(C1CCC(=O)N(C(=O)OC(C)(C)C)C1=O)C2=O ; CN(CCCCCCCC(=O)O)c1cccc2c1CN(C1CCC(=O)NC1=O)C2=O ; CCc1c[nH]c2ncc(-c3cccc(N4CCNCC4=O)c3)c(Cl)c12 ; CCc1c[nH]c2ncc(-c3cccc(N4CCN(C(=O)CCCCCCCN(C)c5cccc6c5CN(C5CCC(=O)NC5=O)C6=O)CC4=O)c3)c(Cl)c12]

Step 1. (General Step H) Preparation of tert-butyl 3-(4-((8-(tert-butoxy)-8-oxooctyl)amino)-1-oxoisoindolin-2-yl)-2,6-dioxopiperidine-1-carboxylate: To a solution of tert-butyl 8-((2-(2,6-dioxopiperidin-3-yl)-1-oxoisoindolin-4-yl)amino) octanoate (200 mg, 0.43 mmol) in THF (5 mL) were added 4-DMAP (14 mg, 0.043 mmol), Boc₂O (105 mg, 0.48 mmol) and TEA (88 mg, 0.87 mmol). The reaction mixture was stirred at room temperature for 16 hrs. The reaction mixture was concentrated and purified by silica gel column chromatography (PE:EA=5:1) to give the product as a yellow oil (100 mg, 41%). Mass (m/z): 580.0 [M+Na]⁺.

Step 2. (General Step I) Preparation of tert-butyl 3-(4-((8-(tert-butoxy)-8-oxooctyl)(methyl)amino)-1-oxoisoindolin-2-yl)-2,6-dioxopiperidine-1-carboxylate: To a solution of tert-butyl 3-(4-((8-(tert-butoxy)-8-oxooctyl)amino)-1-oxoisoindolin-2-yl)-2,6-dioxopiperidine-1-carboxylate (100 mg, 0.18 mmol) in DCM (3 mL) were added Paraformaldehyde (54 mg, 1.79 mmol) and Glacial acetic acid (1 drop). The reaction mixture was stirred at rt for 4 hrs. Then NaBH(AcO)₃ (76 mg, 0.35 mmol) was added and the mixture was stirred at rt for 16 hrs. The reaction mixture was concentrated and purified by silica gel column chromatography (PE:EA=5:1) to give the product as a yellow oil (100 mg, 97%). Mass (m/z): 572.0 [M+H]⁺.

Step 3. Preparation of 8-((2-(2,6-dioxopiperidin-3-yl)-1-oxoisoindolin-4-yl)(methyl)amino) octanoic acid: Following General Step B2, 8-((2-(2,6-dioxopiperidin-3-yl)-1-oxoisoindolin-4-yl)(methyl)amino) octanoic acid was prepared as a yellow oil (70 mg, 96%). Mass (m/z): 416.0 [M+H]⁺.

Step 4. Preparation of 3-(4-((8-(4-(3-(4-chloro-3-ethyl-1H-pyrrolo[2,3-b]pyridin-5-yl)phenyl)-3-oxopiperazin-1-yl)-8-oxooctyl)(methyl)amino)-1-oxoisoindolin-2-yl)piperidine-2,6-dione: Following General Step F, 3-(4-((8-(4-(3-(4-chloro-3-ethyl-1H-pyrrolo[2,3-b]pyridin-5-yl)phenyl)-3-oxopiperazin-1-yl)-8-oxooctyl)(methyl)amino)-1-oxoisoindolin-2-yl)piperidine-2,6-dione was prepared as a white solid (16.8 mg, 16%). Mass (m/z): 751.7 [M+H]⁺.

### Example 12: 3-(4-(2-(2-(3-(4-(3-(4-chloro-3-ethyl-1H-pyrrolo[2,3-b]pyridin-5-yl)phenyl)-3-oxopiperazin-1-yl)-3-oxopropoxy)ethoxy)ethoxy)-1-oxoisoindolin-2-yl)piperidine-2,6-dione

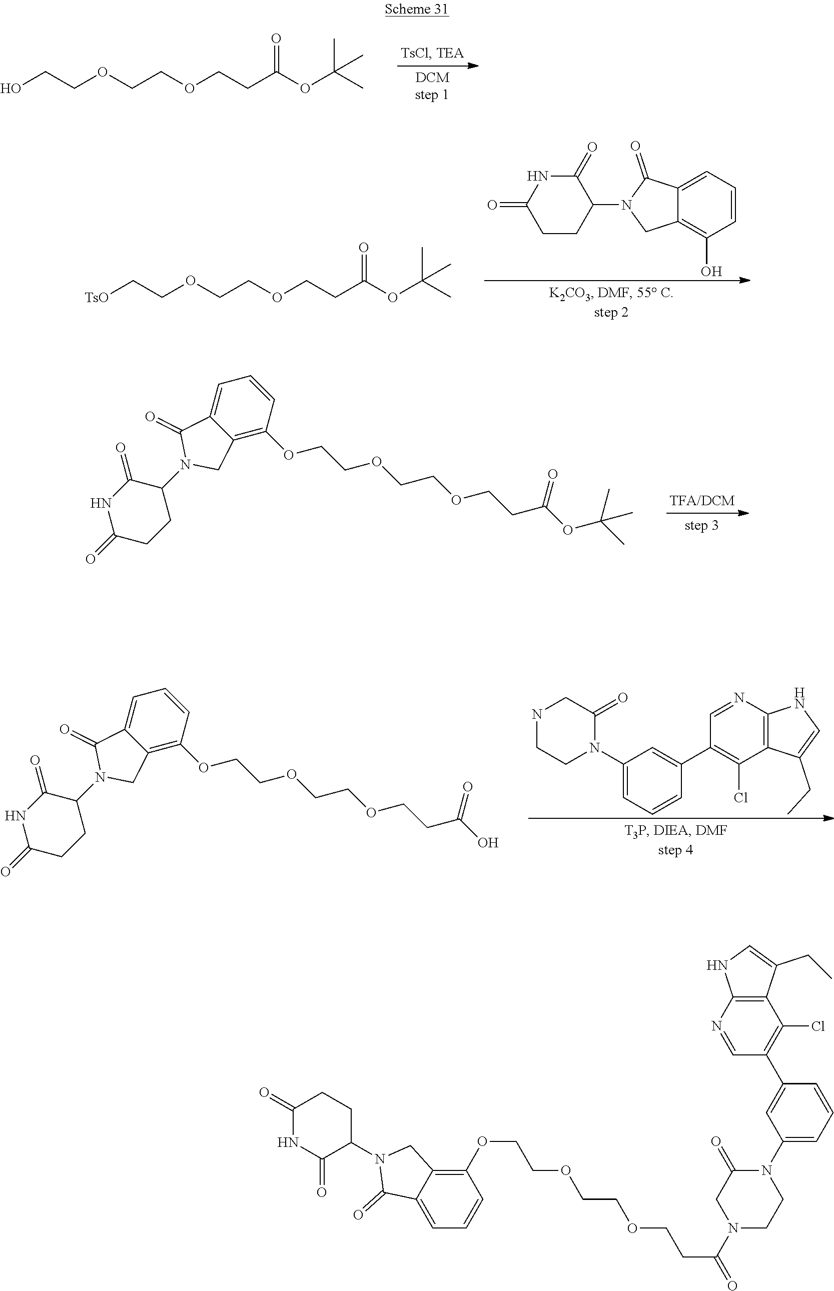

Step 1. Preparation of tert-butyl 3-(2-(2-(tosyloxy)ethoxy)ethoxy) propanoate: Following General Step D, tert-butyl 3-(2-(2-(tosyloxy)ethoxy)ethoxy) propanoate was prepared as a colorless oil (1.5 g, 90%). Mass (m/z): 411.1 [M+Na]⁺.

Step 2. Preparation of tert-butyl 3-(2-(2-((2-(2,6-dioxopiperidin-3-yl)-1-oxoisoindolin-4-yl)oxy)ethoxy)ethoxy) propanoate: Following General Step E, tert-butyl 3-(2-(2-((2-(2,6-dioxopiperidin-3-yl)-1-oxoisoindolin-4-yl)oxy)ethoxy)ethoxy) propanoate was prepared as a white solid (276 mg, 45%). Mass (m/z): 421.0 [M−55]⁺.

Step 3. Preparation of 3-(2-(2-((2-(2,6-dioxopiperidin-3-yl)-1-oxoisoindolin-4-yl)oxy)ethoxy)ethoxy) propanoic acid: Following General Step B2, 3-(2-(2-((2-(2,6-dioxopiperidin-3-yl)-1-oxoisoindolin-4-yl)oxy)ethoxy)ethoxy) propanoic acid was prepared as a white solid (110 mg, 98%). Mass (m/z): 421.1 [M+H]⁺.

Step 4. Preparation of 3-(4-(2-(2-(3-(4-(3-(4-chloro-3-ethyl-1H-pyrrolo[2,3-b]pyridin-5-yl)phenyl)-3-oxopiperazin-1-yl)-3-oxopropoxy)ethoxy)ethoxy)-1-oxoisoindolin-2-yl)piperidine-2,6-dione: Following General Step F, 3-(4-(2-(2-(3-(4-(3-(4-chloro-3-ethyl-1H-pyrrolo[2,3-b]pyridin-5-yl)phenyl)-3-oxopiperazin-1-yl)-3-oxopropoxy)ethoxy)ethoxy)-1-oxoisoindolin-2-yl)piperidine-2,6-dione was prepared as a white solid (20 mg, 44%). Mass (m/z): 757.0 [M+H]⁺.

### Example 13: 3-(4-(4-((2-(3-(4-(3-(4-chloro-3-ethyl-1H-pyrrolo[2,3-b]pyridin-5-yl)phenyl)-3-oxopiperazin-1-yl)-3-oxopropoxy)ethoxy)methyl)-1H-1,2,3-triazol-1-yl)-1-oxoisoindolin-2-yl)piperidine-2,6-dione

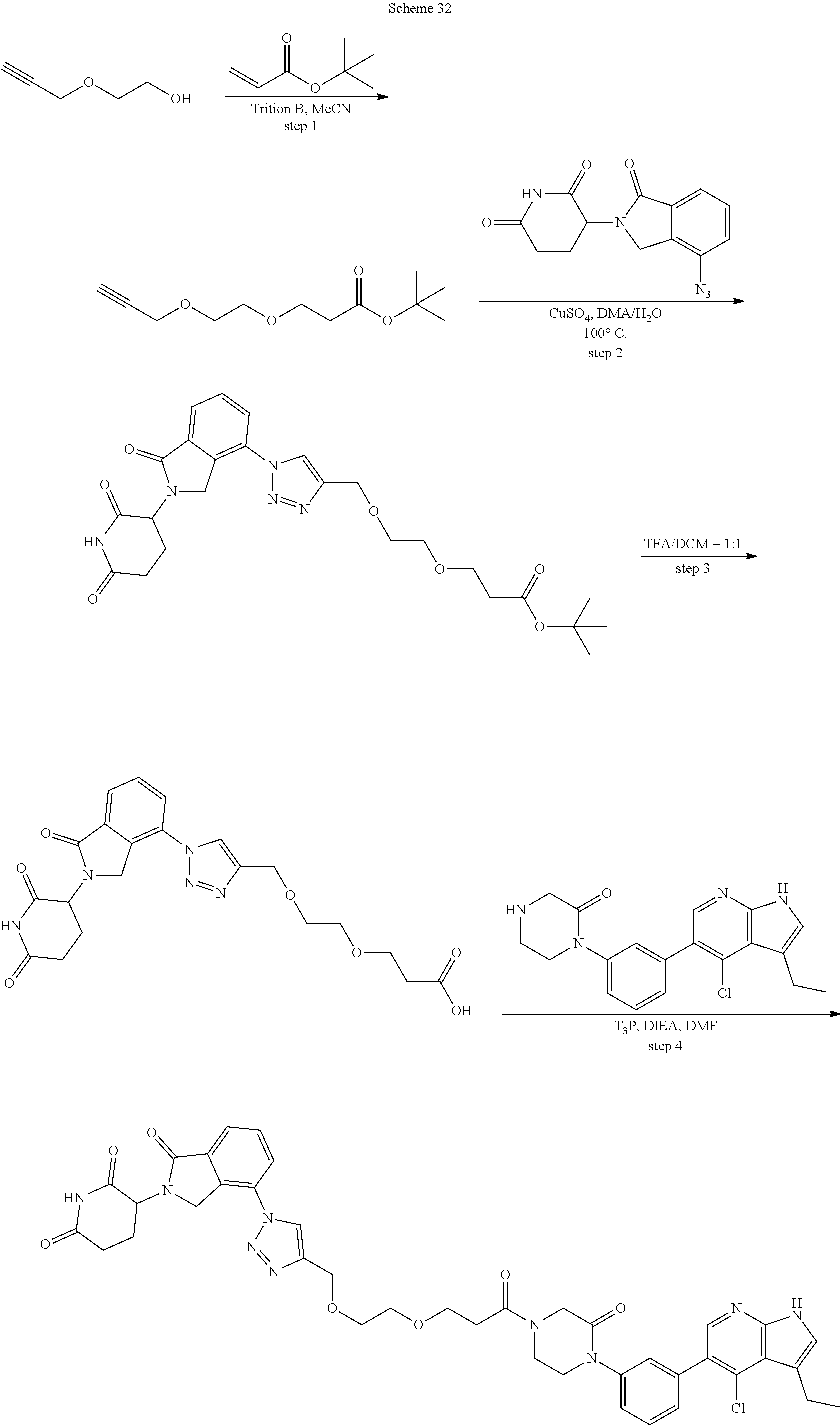

Step 1. Preparation of tert-butyl 3-[2-(prop-2-yn-1-yloxy)ethoxy]propanoate: To a solution of 2-(prop-2-yn-1-yloxy) ethanol (976 mg, 9.75 mmol), tert-butyl prop-2-enoate (500 mg, 3.90 mmol) in MeCN was added Trition B (40 wt. % in water) (24 mg, 0.14 mmol) and then stirred under nitrogen at 25° C. for 72 hrs. Remove the solvent under reduced pressure, purify the crude product by column chromatography, eluting with DCM:MeOH=20:1 to give tert-butyl 3-[2-(prop-2-yn-1-yloxy)ethoxy]propanoate as a light yellow oil (470 mg, 50% yield). Mass (m/z): 251.1 [M+Na]⁺.

Step 2. Preparation of tert-butyl 3-[2-({1-[2-(2,6-dioxopiperidin-3-yl)-1-oxo-3H-isoindol-4-yl]-1,2,3-triazol-4-yl}methoxy)ethoxy]propanoate: To a stirred solution of tert-butyl 3-[2-(prop-2-yn-1-yloxy)ethoxy]propanoate (470 mg, 2.06 mmol), 3-(4-azido-1-oxo-3H-isoindol-2-yl)piperidine-2,6-dione (881 mg, 3.09 mmol) in DMA under nitrogen at 100° C. was added a solution of CuSO₄ (153 mg, 0.62 mmol), L-Ascorbic acid sodium salt (122.7 mg, 0.62 mmol) in water. The reaction mixture was stirred at 100° C. for 12 hrs. The reaction was cooled to room temperature, extracted with EA (200 mL×2), wash with water (20 mL×2) and saturated brine. Organic layer was concentrated, the residue was purified by column chromatography with DCM:MeOH=30:1 to give tert-butyl 3-[2-({1-[2-(2,6-dioxopiperidin-3-yl)-1-oxo-3H-isoindol-4-yl]-1,2,3-triazol-4-yl}methoxy)ethoxy]propanoate as a brown solid (600 mg, 30% yield). Mass (m/z): 514.2 [M+H]⁺.

Step 3. Preparation of 3-[2-({1-[2-(2,6-dioxopiperidin-3-yl)-1-oxo-3H-isoindol-4-yl]-1,2,3-triazol-4-yl}methoxy)ethoxy]propanoic acid: Following General Step B2, 3-[2-({1-[2-(2,6-dioxopiperidin-3-yl)-1-oxo-3H-isoindol-4-yl]-1,2,3-triazol-4-yl}methoxy)ethoxy]propanoic acid was prepared as a light yellow solid (400 mg, 40% yield). Mass (m/z): 458.1 [M+H]⁺.

Step 4. Preparation of 3-(4-(4-((2-(3-(4-(3-(4-chloro-3-ethyl-1H-pyrrolo[2,3-b]pyridin-5-yl)phenyl)-3-oxopiperazin-1-yl)-3-oxopropoxy)ethoxy)methyl)-1H-1,2,3-triazol-1-yl)-1-oxoisoindolin-2-yl)piperidine-2,6-dione): Following General Step F, 3-(4-(4-((2-(3-(4-(3-(4-chloro-3-ethyl-1H-pyrrolo[2,3-b]pyridin-5-yl)phenyl)-3-oxopiperazin-1-yl)-3-oxopropoxy)ethoxy)methyl)-1H-1,2,3-triazol-1-yl)-1-oxoisoindolin-2-yl)piperidine-2,6-dione was prepared as a light yellow solid (60 mg, 34% yield). Mass (m/z): 793.9 [M+H]⁺.

### Example 14: 3-(4-((6-(4-(3-(4-chloro-3-ethyl-1H-pyrrolo[2,3-b]pyridin-5-yl)phenyl)-3-oxopiperazin-1-yl)-6-oxohexyl)(methyl)amino)-1-oxoisoindolin-2-yl)piperidine-2,6-dione

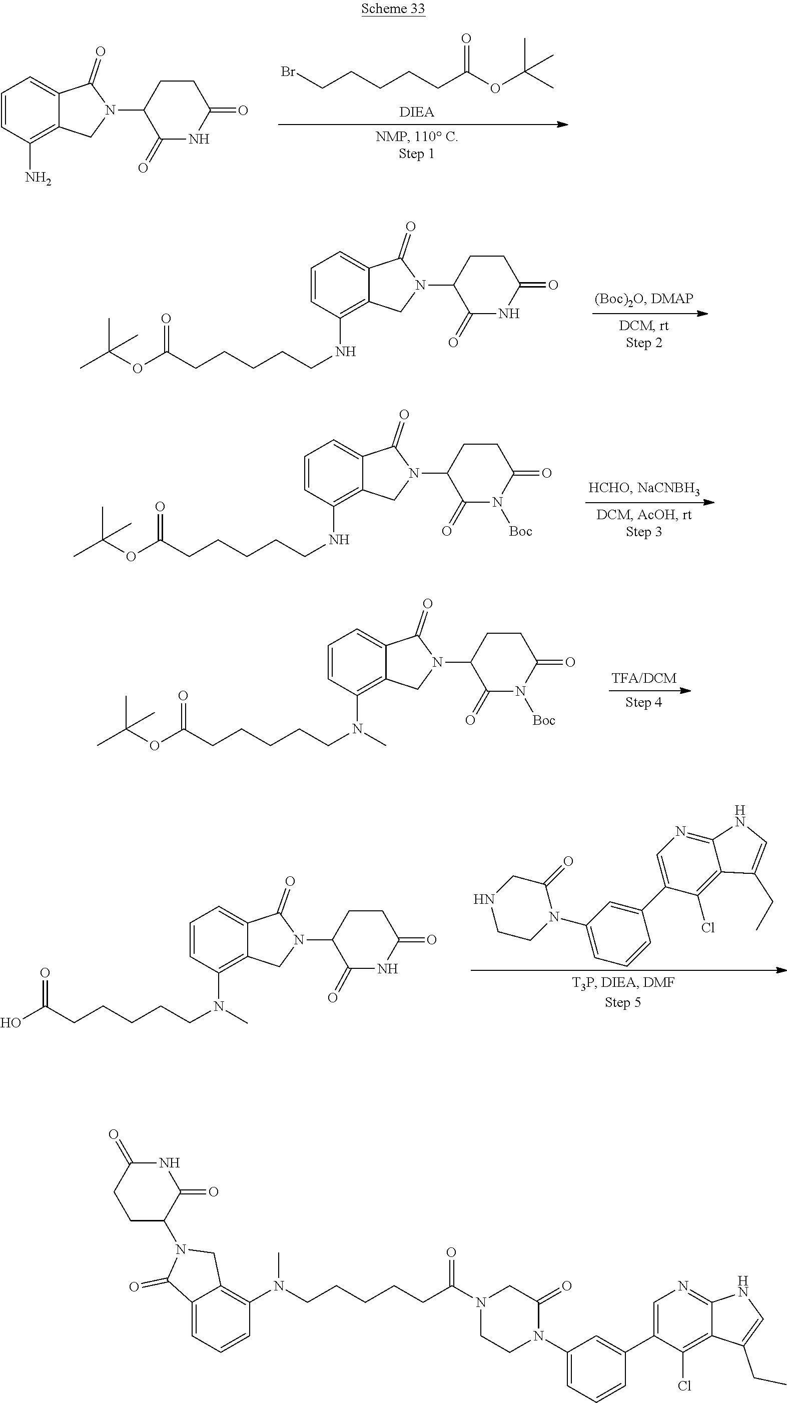

Step 1. Preparation of tert-butyl 6-((2-(2,6-dioxopiperidin-3-yl)-1-oxoisoindolin-4-yl)amino) hexanoate: Following General Step G1, tert-butyl 6-((2-(2,6-dioxopiperidin-3-yl)-1-oxoisoindolin-4-yl)amino) hexanoate was prepared as a yellow solid (854 mg, 51%). Mass (m/z): 451.9 [M+Na]⁺.

Step 2. Preparation of tert-butyl 3-(4-((6-(tert-butoxy)-6-oxohexyl)amino)-1-oxoisoindolin-2-yl)-2,6-dioxopiperidine-1-carboxylate: Following General Step H, tert-butyl 3-(4-((6-(tert-butoxy)-6-oxohexyl)amino)-1-oxoisoindolin-2-yl)-2,6-dioxopiperidine-1-carboxylate was prepared as a yellow solid (550 mg, 53%). Mass (m/z): 552.8 [M+Na]⁺.

Step 3. Preparation of tert-butyl 3-(4-((6-(tert-butoxy)-6-oxohexyl)(methyl)amino)-1-oxoisoindolin-2-yl)-2,6-dioxopiperidine-1-carboxylate: Following General Step I, tert-butyl 3-(4-((6-(tert-butoxy)-6-oxohexyl)(methyl)amino)-1-oxoisoindolin-2-yl)-2,6-dioxopiperidine-1-carboxylate was prepared as a yellow oil (244 mg, 79%). Mass (m/z): 543.8 [M+H]⁺.

Step 4. Preparation of 6-((2-(2,6-dioxopiperidin-3-yl)-1-oxoisoindolin-4-yl)(methyl)amino) hexanoic acid: Following General Step B2, 6-((2-(2,6-dioxopiperidin-3-yl)-1-oxoisoindolin-4-yl)(methyl)amino) hexanoic acid was prepared as a yellow oil (300 mg, 86%). Mass (m/z): 387.8 [M+H]⁺.

Step 5. Preparation of 3-(4-((6-(4-(3-(4-chloro-3-ethyl-1H-pyrrolo[2,3-b]pyridin-5-yl)phenyl)-3-oxopiperazin-1-yl)-6-oxohexyl)(methyl)amino)-1-oxoisoindolin-2-yl)piperidine-2,6-dione: Following General Step F, 3-(4-((6-(4-(3-(4-chloro-3-ethyl-1H-pyrrolo[2,3-b]pyridin-5-yl)phenyl)-3-oxopiperazin-1-yl)-6-oxohexyl)(methyl)amino)-1-oxoisoindolin-2-yl)piperidine-2,6-dione was prepared as a white solid (20 mg, 15%). Mass (m/z): 723.6 [M+H]⁺.

### Example 15: 3-(4-((4-(4-(3-(4-chloro-3-ethyl-1H-pyrrolo[2,3-b]pyridin-5-yl)phenyl)-3-oxopiperazin-1-yl)-4-oxobutyl)(methyl)amino)-1-oxoisoindolin-2-yl)piperidine-2,6-dione

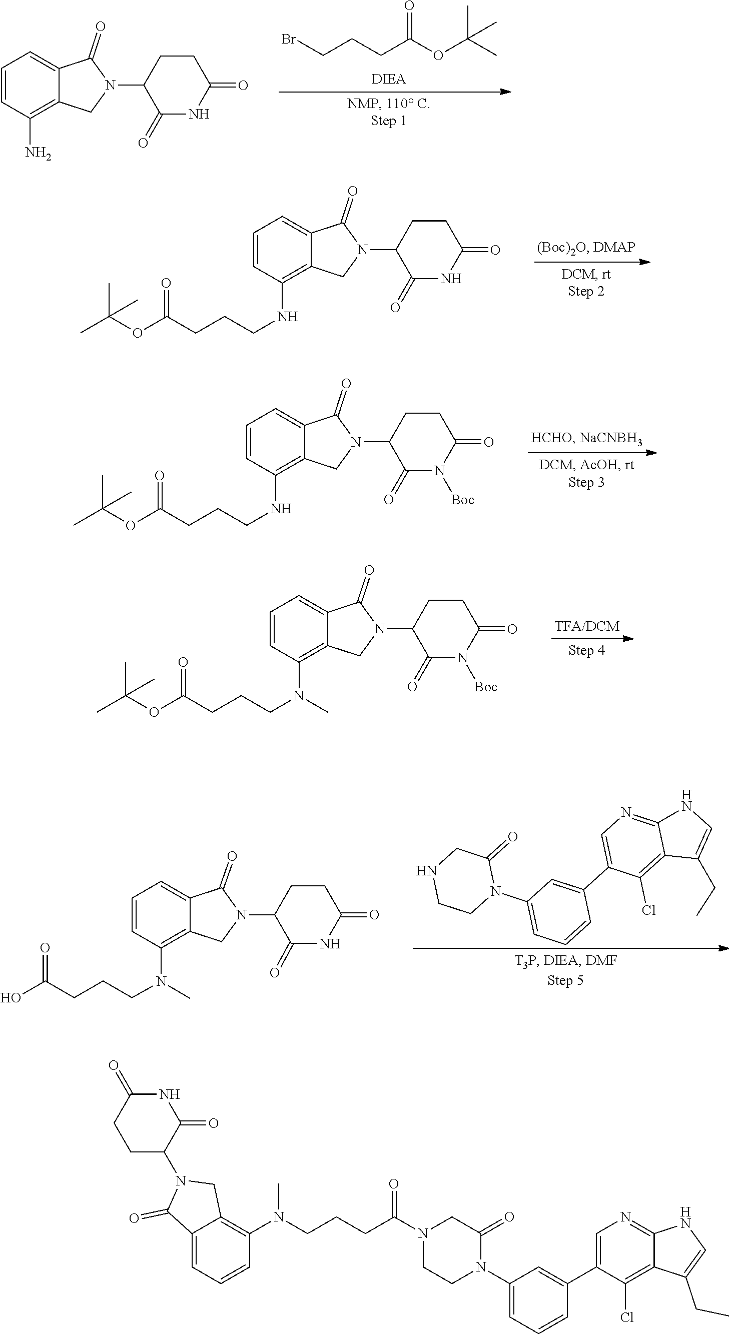

Step 1. Preparation of tert-butyl 4-((2-(2,6-dioxopiperidin-3-yl)-1-oxoisoindolin-4-yl)amino) butanoate: Following General Step G1, tert-butyl 4-((2-(2,6-dioxopiperidin-3-yl)-1-oxoisoindolin-4-yl)amino) butanoate was prepared as a yellow solid (1.1 g, 47%). Mass (m/z): 401.8 [M+H]⁺.

Step 2. Preparation of tert-butyl 3-(4-((4-(tert-butoxy)-4-oxobutyl)amino)-1-oxoisoindolin-2-yl)-2,6-dioxopiperidine-1-carboxylate: Following General Step H, tert-butyl 3-(4-((4-(tert-butoxy)-4-oxobutyl)amino)-1-oxoisoindolin-2-yl)-2,6-dioxopiperidine-1-carboxylate was prepared as a yellow oil (654 mg, 47%). Mass (m/z): 523.9 [M+Na]⁺.

Step 3. Preparation of tert-butyl 3-(4-((4-(tert-butoxy)-4-oxobutyl)(methyl)amino)-1-oxoisoindolin-2-yl)-2,6-dioxopiperidine-1-carboxylate: Following General Step I, tert-butyl 3-(4-((4-(tert-butoxy)-4-oxobutyl)(methyl)amino)-1-oxoisoindolin-2-yl)-2,6-dioxopiperidine-1-carboxylate was prepared as a yellow oil (272 mg, 88%). Mass (m/z): 515.8 [M+H]⁺.

Step 4. Preparation of 4-((2-(2,6-dioxopiperidin-3-yl)-1-oxoisoindolin-4-yl)(methyl)amino) butanoic acid: Following General Step B2, 4-((2-(2,6-dioxopiperidin-3-yl)-1-oxoisoindolin-4-yl)(methyl)amino) butanoic acid was prepared as a yellow oil (310 mg, 79%). Mass (m/z): 359.9 [M+H]⁺.

Step 5. Preparation of 3-(4-((4-(4-(3-(4-chloro-3-ethyl-1H-pyrrolo[2,3-b]pyridin-5-yl)phenyl)-3-oxopiperazin-1-yl)-4-oxobutyl)(methyl)amino)-1-oxoisoindolin-2-yl)piperidine-2,6-dione: Following General Step F, 3-(4-((4-(4-(3-(4-chloro-3-ethyl-1H-pyrrolo[2,3-b]pyridin-5-yl)phenyl)-3-oxopiperazin-1-yl)-4-oxobutyl)(methyl)amino)-1-oxoisoindolin-2-yl)piperidine-2,6-dione was prepared as a white solid (48 mg, 8%). Mass (m/z): 695.7 [M+H]⁺.

### Example 16: 5-(4-(2-(4-(3-(4-chloro-3-ethyl-1H-pyrrolo[2,3-b]pyridin-5-yl)phenyl)-3-oxopiperazin-1-yl)-2-oxoethoxy)piperidin-1-yl)-2-(2,6-dioxopiperidin-3-yl)isoindoline-1,3-dione

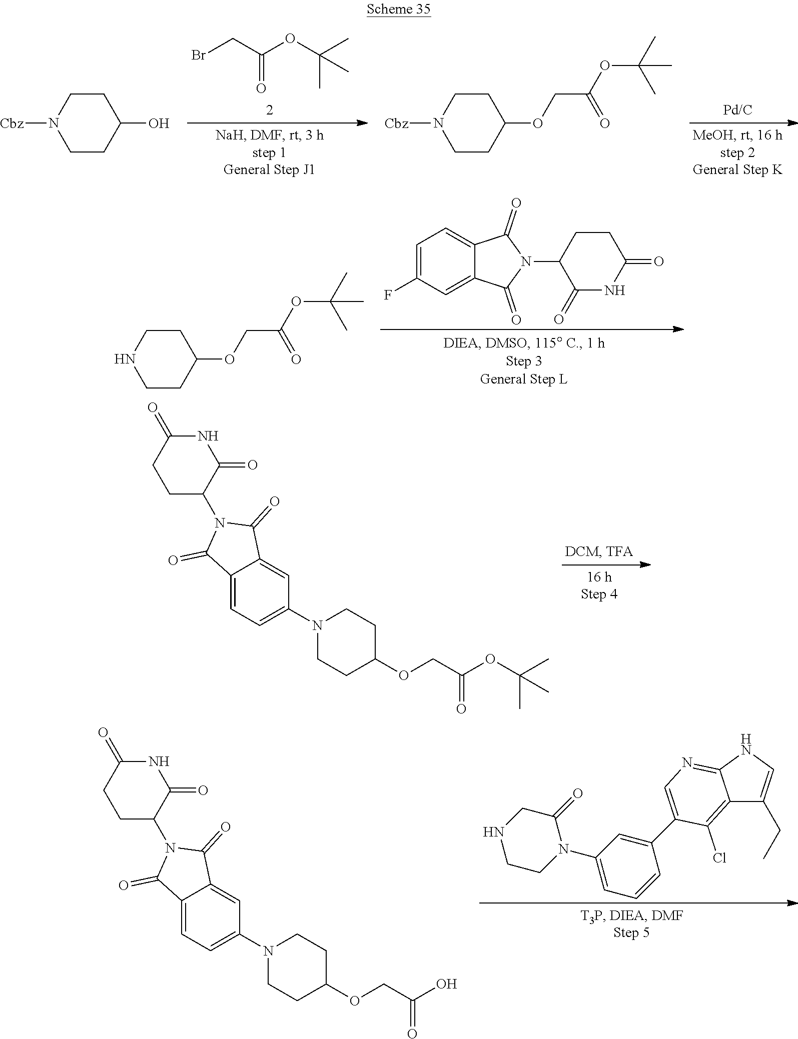

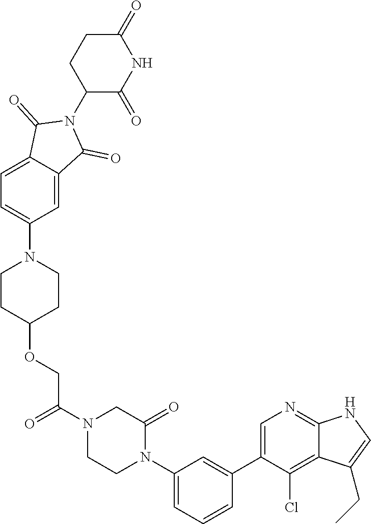 [SMILES: CCc1c[nH]c2ncc(-c3cccc(N4CCN(C(=O)COC5CCN(c6ccc7c(c6)C(=O)N(C6CCC(=O)NC6=O)C7=O)CC5)CC4=O)c3)c(Cl)c12]

Step 1. (General Step J1) Preparation of benzyl 4-(2-(tert-butoxy)-2-oxoethoxy)piperidine-1-carboxylate: To a mixture of NaH (60% in oil, 920 mg, 38.3 mmol) in DMF (50 mL) was added benzyl 4-hydroxypiperidine-1-carboxylate (5.0 g, 0.021 mol) at 0° C. The reaction was stirred at 0° C. for 15 min and then tert-butyl 2-bromoacetate (4.57 g, 0.023 mol) was added. The reaction was stirred at rt for 3 hrs. The reaction mixture was quenched with saturated NH₄Cl solution (80 mL), then extracted with EtOAc (100 mL×3). The combined organic layers were washed with saturated NaCl solution for three times, dried over Na₂SO₄ and concentrated under reduced pressure. The residue was purified via Flash Chromatography and was eluted with PE/EtOAc (0-10%) to give benzyl 4-(2-(tert-butoxy)-2-oxoethoxy)piperidine-1-carboxylate as a yellow oil (3.67 g, 30%). Mass (m/z): 294.0 [M−55]⁺.

Step 2. (General Step K) Preparation of tert-butyl 2-(piperidin-4-yloxy)acetate: To a mixture of benzyl 4-(2-(tert-butoxy)-2-oxoethoxy)piperidine-1-carboxylate (3.70 g, 10.6 mmol) in MeOH (40 mL) was added 10% Pd/C (370 mg, 10% wt/wt). The reaction was degassed with N₂ for 3 times, and then stirred at rt under H₂ (0.1 MPa) for 16 hrs. The reaction mixture was filtered through Celite, and the filtrate was concentrated under reduced pressure to give tert-butyl 2-(piperidin-4-yloxy)acetate as a yellow oil (2.10 g, 90%).

Step 3. (General Step L) Preparation of tert-butyl 2-({1-[2-(2,6-dioxopiperidin-3-yl)-1,3-dioxoisoindol-5-yl]piperidin-4-yl}oxy)acetate: To a mixture of tert-butyl 2-(piperidin-4-yloxy)acetate (500 mg, 2.32 mmol) in DMSO (6.0 mL) were added DIEA (360 mg, 2.78 mmol) and 2-(2,6-dioxopiperidin-3-yl)-5-fluoroisoindole-1,3-dione (769 mg, 2.78 mmol). The reaction was stirred at 115° C. for 1 hr. The reaction mixture was diluted with H₂O (20 mL), then extracted with EtOAc (50 mL×3). The combined organic layers were washed with saturated NaCl solution for three times, dried over Na₂SO₄ and concentrated under reduced pressure. The residue was purified via Flash Chromatography and was eluted with PE/EtOAc (0-50%) to give tert-butyl 2-({1-[2-(2,6-dioxopiperidin-3-yl)-1,3-dioxoisoindol-5-yl]piperidin-4-yl}oxy)acetate as a white solid (480 mg, 39%). Mass (m/z): 471.9 [M+H]⁺.

Step 4. Preparation of ({1-[2-(2,6-dioxopiperidin-3-yl)-1,3-dioxoisoindol-5-yl]piperidin-4-yl}oxy) acetic acid: Following General Step B2, ({1-[2-(2,6-dioxopiperidin-3-yl)-1,3-dioxoisoindol-5-yl]piperidin-4-yl}oxy) acetic acid was prepared as a brown oil (400 mg, 85%), which was used into next step directly without further purification. Mass (m/z): 415.9 [M+H]⁺.

Step 5. Preparation of 5-(4-(2-(4-(3-(4-chloro-3-ethyl-1H-pyrrolo[2,3-b]pyridin-5-yl)phenyl)-3-oxopiperazin-1-yl)-2-oxoethoxy)piperidin-1-yl)-2-(2,6-dioxopiperidin-3-yl)isoindoline-1,3-dione: Following General Step F, 5-(4-(2-(4-(3-(4-chloro-3-ethyl-1H-pyrrolo[2,3-b]pyridin-5-yl)phenyl)-3-oxopiperazin-1-yl)-2-oxoethoxy)piperidin-1-yl)-2-(2,6-dioxopiperidin-3-yl)isoindoline-1,3-dione was prepared as a yellow solid (43 mg, 13%). Mass (m/z): 751.7 [M+H]⁺.

### Example 17: 4-(4-(2-(4-(3-(4-chloro-3-ethyl-1H-pyrrolo[2,3-b]pyridin-5-yl)phenyl)-3-oxopiperazin-1-yl)-2-oxoethoxy)piperidin-1-yl)-2-(2,6-dioxopiperidin-3-yl)isoindoline-1,3-dione

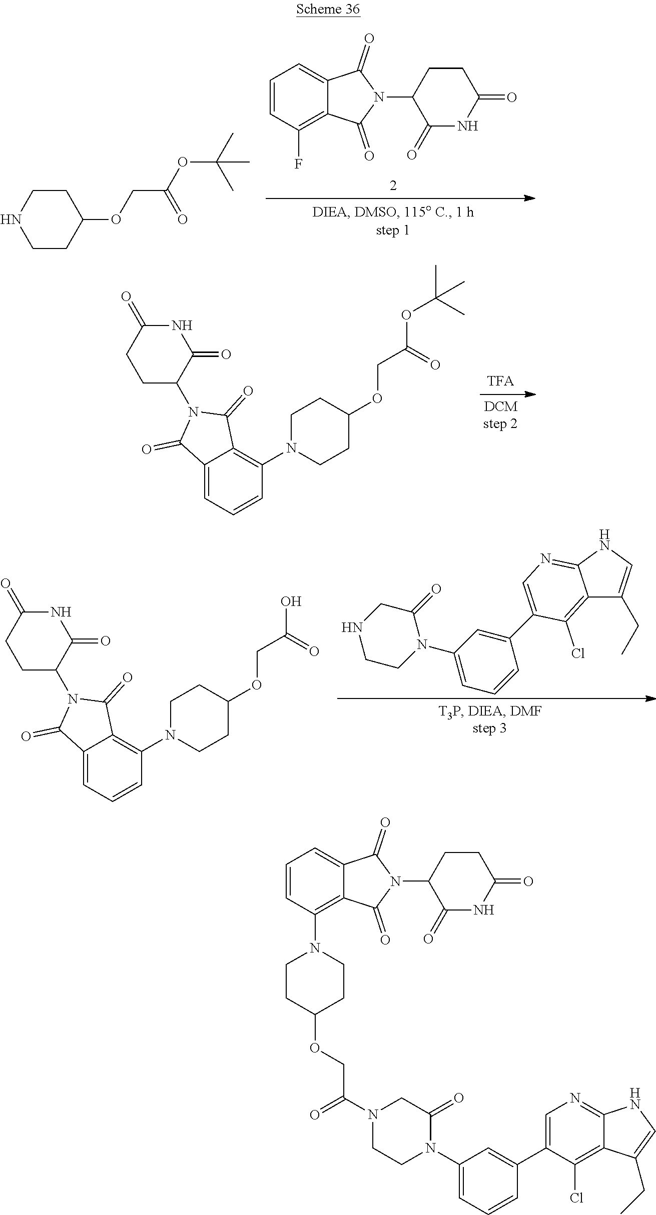

Step 1. Preparation of tert-butyl 2-({1-[2-(2,6-dioxopiperidin-3-yl)-1,3-dioxoisoindol-4-yl]piperidin-4-yl}oxy)acetate: Following General Step L, tert-butyl 2-({1-[2-(2,6-dioxopiperidin-3-yl)-1,3-dioxoisoindol-4-yl]piperidin-4-yl}oxy)acetate was prepared as a yellow oil (700 mg, 48%). Mass (m/z): 471.9 [M+H]⁺.

Step 2. Preparation of ({1-[2-(2,6-dioxopiperidin-3-yl)-1,3-dioxoisoindol-4-yl]piperidin-4-yl}oxy) acetic acid: Following General Step B2, ({1-[2-(2,6-dioxopiperidin-3-yl)-1,3-dioxoisoindol-4-yl]piperidin-4-yl}oxy) acetic acid was prepared as a yellow oil (520 mg, 71%). Mass (m/z): 415.8 [M+H]⁺.

Step 3. Preparation of 4-(4-(2-(4-(3-(4-chloro-3-ethyl-1H-pyrrolo[2,3-b]pyridin-5-yl)phenyl)-3-oxopiperazin-1-yl)-2-oxoethoxy)piperidin-1-yl)-2-(2,6-dioxopiperidin-3-yl)isoindoline-1,3-dione: Following General Step F, 4-(4-(2-(4-(3-(4-chloro-3-ethyl-1H-pyrrolo[2,3-b]pyridin-5-yl)phenyl)-3-oxopiperazin-1-yl)-2-oxoethoxy)piperidin-1-yl)-2-(2,6-dioxopiperidin-3-yl)isoindoline-1,3-dione was prepared as a white solid (22.5 mg, 15%). Mass (m/z): 751.7 [M+H]⁺.

### Example 18: 4-(3-(2-(4-(3-(4-chloro-3-ethyl-1H-pyrrolo[2,3-b]pyridin-5-yl)phenyl)-3-oxopiperazin-1-yl)-2-oxoethoxy) azetidin-1-yl)-2-(2,6-dioxopiperidin-3-yl)isoindoline-1,3-dione

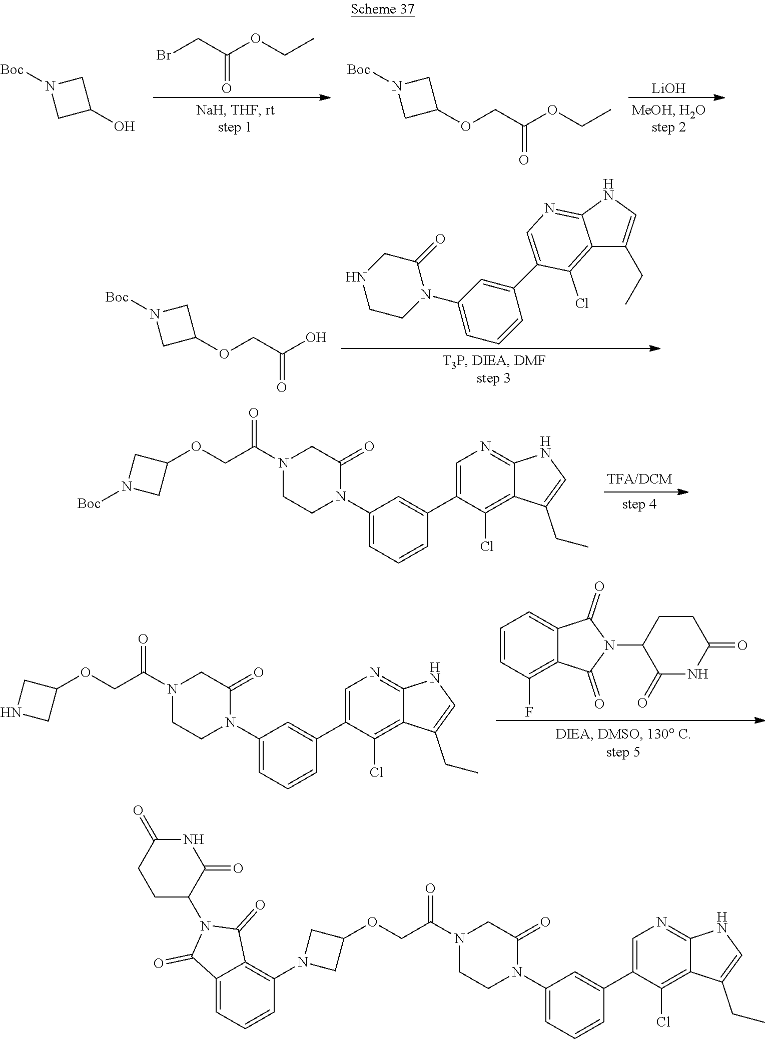

Step 1. Preparation of tert-butyl 3-(2-ethoxy-2-oxoethoxy) azetidine-1-carboxylate: Following General Step J1, tert-butyl 3-(2-ethoxy-2-oxoethoxy) azetidine-1-carboxylate was prepared as a yellow oil (900 mg, 11%).

Step 2. Preparation of 2-((1-(tert-butoxycarbonyl) azetidin-3-yl)oxy) acetic acid: To a solution of tert-butyl 3-(2-ethoxy-2-oxoethoxy) azetidine-1-carboxylate (100 mg, 0.38 mmol) in the mixed solvent of MeOH and H₂O (3 mL, 2:1 (v/v)) was added LiOH H₂O (80.6 mg, 1.92 mmol). The reaction mixture was stirred at room temperature for 16 hrs. The reaction solution was acidified to pH 3 with 1M HCl solution. The reaction solution was extracted with EA (25 mL×2). The combined organic layers were washed with brine (25 mL×2), dried over Na₂SO₄. The solvent was removed under reduced pressure and freezing to give 2-((1-(tert-butoxycarbonyl) azetidin-3-yl)oxy) acetic acid as a yellow oil (40 mg, 36%). Mass (m/z): 230.0 [M−H]⁻.

Step 3. Preparation of tert-butyl 3-(2-(4-(3-(4-chloro-3-ethyl-1H-pyrrolo[2,3-b]pyridin-5-yl)phenyl)-3-oxopiperazin-1-yl)-2-oxoethoxy) azetidine-1-carboxylate: Following General Step F, tert-butyl 3-(2-(4-(3-(4-chloro-3-ethyl-1H-pyrrolo[2,3-b]pyridin-5-yl)phenyl)-3-oxopiperazin-1-yl)-2-oxoethoxy) azetidine-1-carboxylate was prepared as a light-yellow solid (25 mg, 20%). Mass (m/z): 567.9 [M+H]⁺.

Step 4. Preparation of 4-(2-(azetidin-3-yloxy) acetyl)-1-(3-(4-chloro-3-ethyl-1H-pyrrolo[2,3-b]pyridin-5-yl)phenyl)piperazin-2-one: Following General Step B2, 4-(2-(azetidin-3-yloxy) acetyl)-1-(3-(4-chloro-3-ethyl-1H-pyrrolo[2,3-b]pyridin-5-yl)phenyl)piperazin-2-one was prepared as a yellow oil (16 mg, 69%). Mass (m/z): 467.8 [M+H]⁺.

Step 5. Preparation of 4-(3-(2-(4-(3-(4-chloro-3-ethyl-1H-pyrrolo[2,3-b]pyridin-5-yl)phenyl)-3-oxopiperazin-1-yl)-2-oxoethoxy) azetidin-1-yl)-2-(2,6-dioxopiperidin-3-yl)isoindoline-1,3-dione: Following General Step L, 4-(3-(2-(4-(3-(4-chloro-3-ethyl-1H-pyrrolo[2,3-b]pyridin-5-yl)phenyl)-3-oxopiperazin-1-yl)-2-oxoethoxy) azetidin-1-yl)-2-(2,6-dioxopiperidin-3-yl)isoindoline-1,3-dione was prepared as a yellow solid (4 mg, 16%). Mass (m/z): 723.6 [M+H]⁺.

### Example 19: 5-((6-(4-(3-(4-chloro-3-ethyl-1H-pyrrolo[2,3-b]pyridin-5-yl)phenyl)-3-oxopiperazin-1-yl)-6-oxohexyl)amino)-2-(2,6-dioxopiperidin-3-yl)isoindoline-1,3-dione

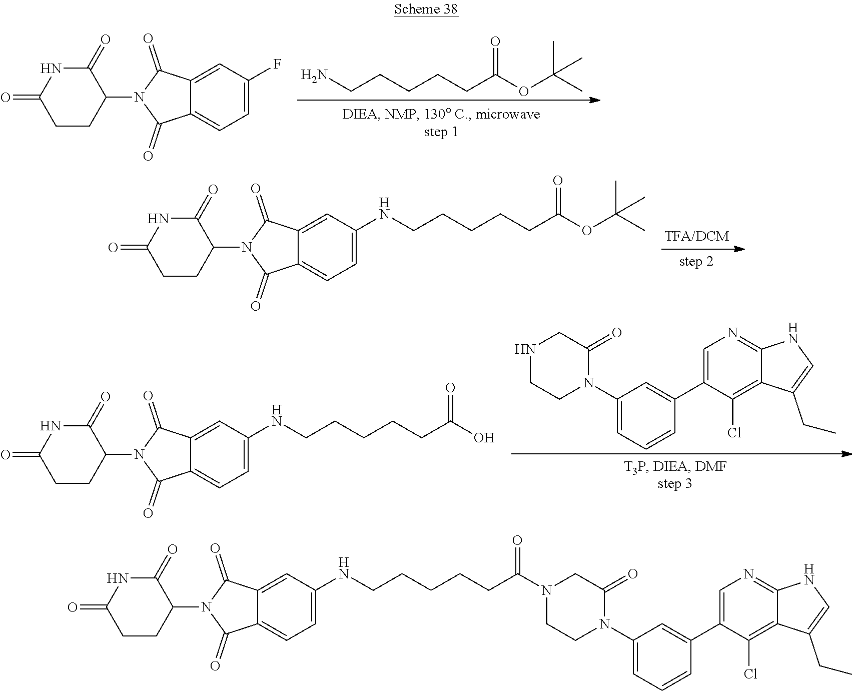

Step 1. Preparation of tert-butyl 6-{[2-(2,6-dioxopiperidin-3-yl)-1,3-dioxoisoindol-5-yl]amino}hexanoate: Following General Step L, tert-butyl 6-{[2-(2,6-dioxopiperidin-3-yl)-1,3-dioxoisoindol-5-yl]amino}hexanoate was prepared as a yellow solid (600 mg, 15%). Mass (m/z): 465.9 [M+Na]⁺.

Step 2. Preparation of 6-{[2-(2,6-dioxopiperidin-3-yl)-1,3-dioxoisoindol-5-yl]amino}hexanoic acid: Following General Step B2, 6-{[2-(2,6-dioxopiperidin-3-yl)-1,3-dioxoisoindol-5-yl]amino}hexanoic acid was prepared as a yellow oil (180 mg, 62%). Mass (m/z): 387.8 [M+H]⁺.

Step 3. Preparation of 5-((6-(4-(3-(4-chloro-3-ethyl-1H-pyrrolo[2,3-b]pyridin-5-yl)phenyl)-3-oxopiperazin-1-yl)-6-oxohexyl)amino)-2-(2,6-dioxopiperidin-3-yl)isoindoline-1,3-dione: Following General Step F, 5-((6-(4-(3-(4-chloro-3-ethyl-1H-pyrrolo[2,3-b]pyridin-5-yl)phenyl)-3-oxopiperazin-1-yl)-6-oxohexyl)amino)-2-(2,6-dioxopiperidin-3-yl)isoindoline-1,3-dione was prepared as a light-yellow solid (23 mg, 6%). Mass (m/z): 723.6 [M+H]⁺.

### Example 20: 4-(4-(3-(4-(3-(4-chloro-3-ethyl-1H-pyrrolo[2,3-b]pyridin-5-yl)phenyl)-3-oxopiperazin-1-yl)-3-oxopropyl)piperazin-1-yl)-2-(2,6-dioxopiperidin-3-yl)isoindoline-1,3-dione

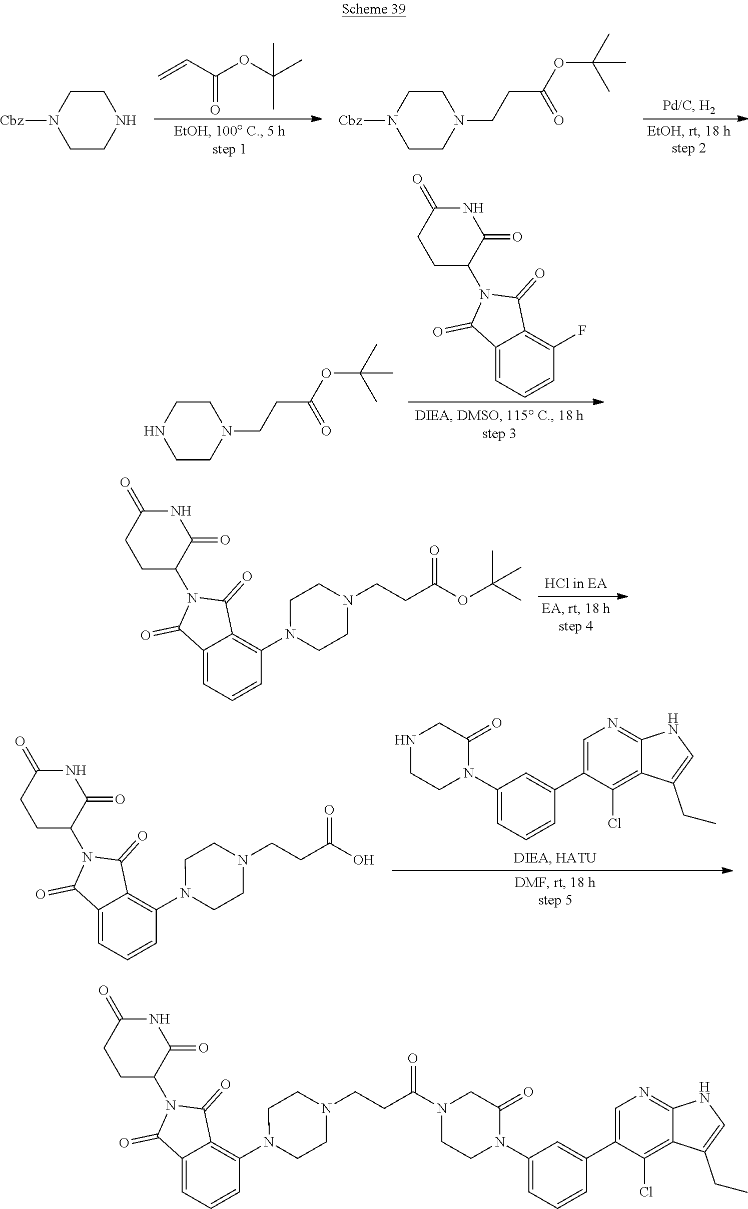

Step 1. Preparation of benzyl 4-(3-(tert-butoxy)-3-oxopropyl)piperazine-1-carboxylate: To a solution of [3-(piperazin-1-yl)phenyl]methyl formate (1 g, 4.5 mmol) in EtOH (3.5 mL) was added tert-butyl prop-2-enoate (0.75 g, 5.8 mmol) under nitrogen. The reaction mixture was stirred at 100° C. for 5 h. After cooled to room temperature, the mixture was filtered and the filter cake was dried to give benzyl 4-(3-(tert-butoxy)-3-oxopropyl)piperazine-1-carboxylate as a grey solid (1.5 g, 95%). MS (ESI) (m/z)=348.9 [M+H]⁺.

Step 2. Preparation of tert-butyl 3-(piperazin-1-yl) propanoate: Following General Step K, tert-butyl 3-(piperazin-1-yl) propanoate was prepared as a colorless oil (0.5 g, 76%). MS (m/z)=215.0 [M+H]⁺.

Step 3. Preparation of tert-butyl 3-(4-(2-(2,6-dioxopiperidin-3-yl)-1,3-dioxoisoindolin-4-yl)piperazin-1-yl) propanoate: Following General Step L, tert-butyl 3-(4-(2-(2,6-dioxopiperidin-3-yl)-1,3-dioxoisoindolin-4-yl)piperazin-1-yl) propanoate was prepared as a yellow solid (1 g, 27%). MS (m/z)=471.2 [M+H]⁺.

Step 4. Preparation of 3-(4-(2-(2,6-dioxopiperidin-3-yl)-1,3-dioxoisoindolin-4-yl)piperazin-1-yl) propanoic acid: Following General Step B1, 3-(4-(2-(2,6-dioxopiperidin-3-yl)-1,3-dioxoisoindolin-4-yl)piperazin-1-yl) propanoic acid was prepared as a yellow solid (0.5 g, 77%). MS (m/z)=415.1 [M+H]⁺.

Step 5. Preparation of 4-(4-(3-(4-(3-(4-chloro-3-ethyl-1H-pyrrolo[2,3-b]pyridin-5-yl)phenyl)-3-oxopiperazin-1-yl)-3-oxopropyl)piperazin-1-yl)-2-(2,6-dioxopiperidin-3-yl) isoindoline-1,3-dione: Following General Step F, 4-(4-(3-(4-(3-(4-chloro-3-ethyl-1H-pyrrolo[2,3-b]pyridin-5-yl)phenyl)-3-oxopiperazin-1-yl)-3-oxopropyl)piperazin-1-yl)-2-(2,6-dioxopiperidin-3-yl)isoindoline-1,3-dione was prepared as a yellow solid (45 mg, 12%). MS: (m/z)=751.2 [M+H]⁺.

### Example 21: 5-(4-(3-(4-(3-(4-chloro-3-ethyl-1H-pyrrolo[2,3-b]pyridin-5-yl)phenyl)-3-oxopiperazin-1-yl)-3-oxopropyl)piperazin-1-yl)-2-(2,6-dioxopiperidin-3-yl)isoindoline-1,3-dione

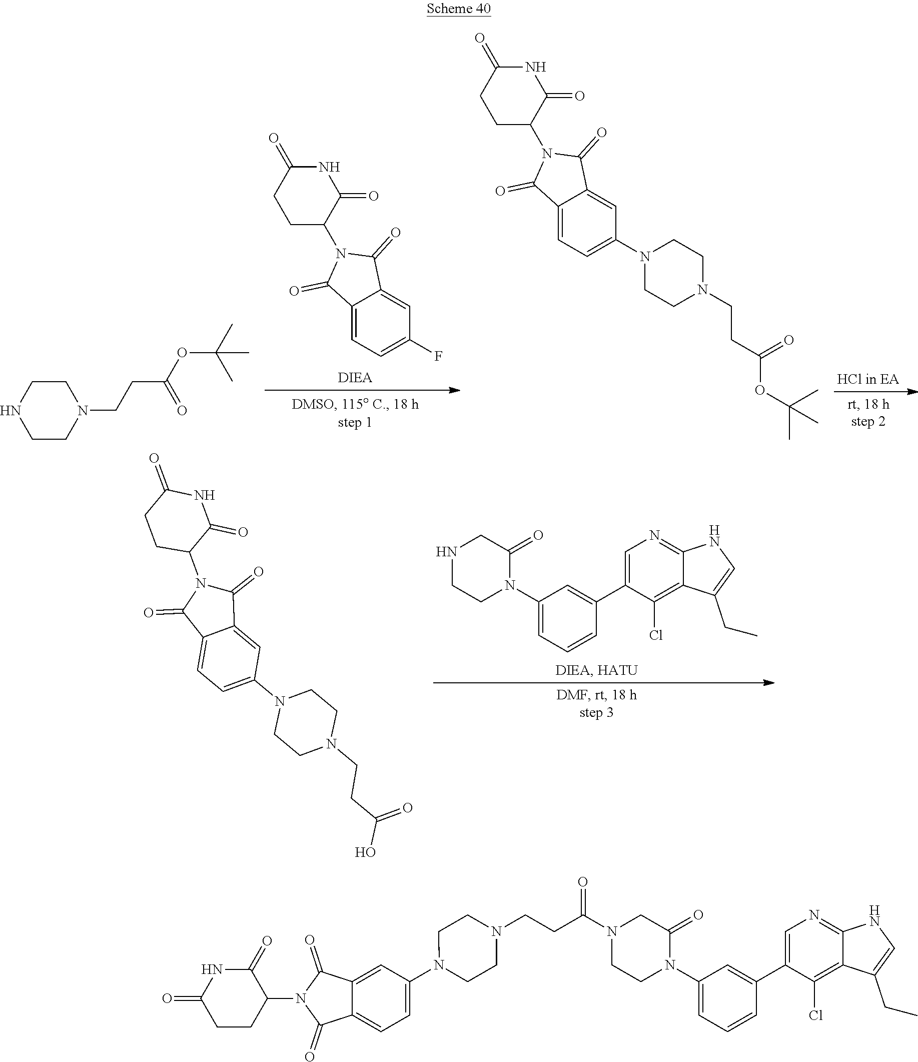 [SMILES: CC(C)(C)OC(=O)CCN1CCNCC1 ; O=C1CCC(N2C(=O)c3ccc(F)cc3C2=O)C(=O)N1 ; CC(C)(C)OC(=O)CCN1CCN(c2ccc3c(c2)C(=O)N(C2CCC(=O)NC2=O)C3=O)CC1 ; O=C(O)CCN1CCN(c2ccc3c(c2)C(=O)N(C2CCC(=O)NC2=O)C3=O)CC1 ; CCc1c[nH]c2ncc(-c3cccc(N4CCNCC4=O)c3)c(Cl)c12 ; CCc1c[nH]c2ncc(-c3cccc(N4CCN(C(=O)CCN5CCN(c6ccc7c(c6)C(=O)N(C6CCC(=O)NC6=O)C7=O)CC5)CC4=O)c3)c(Cl)c12]

Step 1. Preparation of tert-butyl 3-(4-(2-(2,6-dioxopiperidin-3-yl)-1,3-dioxoisoindolin-5-yl)piperazin-1-yl) propanoate: Following General Step L, tert-butyl 3-(4-(2-(2,6-dioxopiperidin-3-yl)-1,3-dioxoisoindolin-5-yl)piperazin-1-yl) propanoate was prepared as a yellow solid (0.5 g, 27%). MS: m/z=471.2 (M+1, ESI+).

Step 2. Preparation of 3-(4-(2-(2,6-dioxopiperidin-3-yl)-1,3-dioxoisoindolin-5-yl) piperazin-1-yl) propanoic acid: Following General Step B1, 3-(4-(2-(2,6-dioxopiperidin-3-yl)-1,3-dioxoisoindolin-5-yl)piperazin-1-yl) propanoic acid was prepared as a yellow solid (200 mg, 43%). MS: m/z=414.9 (M+1, ESI+).

Step 3. Preparation of 5-(4-(3-(4-(3-(4-chloro-3-ethyl-1H-pyrrolo[2,3-b]pyridin-5-yl)phenyl)-3-oxopiperazin-1-yl)-3-oxopropyl)piperazin-1-yl)-2-(2,6-dioxopiperidin-3-yl) isoindoline-1,3-dione: Following General Step F, 5-(4-(3-(4-(3-(4-chloro-3-ethyl-1H-pyrrolo[2,3-b]pyridin-5-yl)phenyl)-3-oxopiperazin-1-yl)-3-oxopropyl)piperazin-1-yl)-2-(2,6-dioxopiperidin-3-yl)isoindoline-1,3-dione was prepared as a yellow solid (27 mg, 14%). MS: m/z=750.8 (M+1, ESI+).

### Example 22: 3-(4-((4-(4-(3-(4-chloro-3-ethyl-1H-pyrrolo[2,3-b]pyridin-5-yl)phenyl)-3-oxopiperazin-1-yl)-4-oxobutyl)(methyl)amino)-1-oxoisoindolin-2-yl)piperidine-2,6-dione

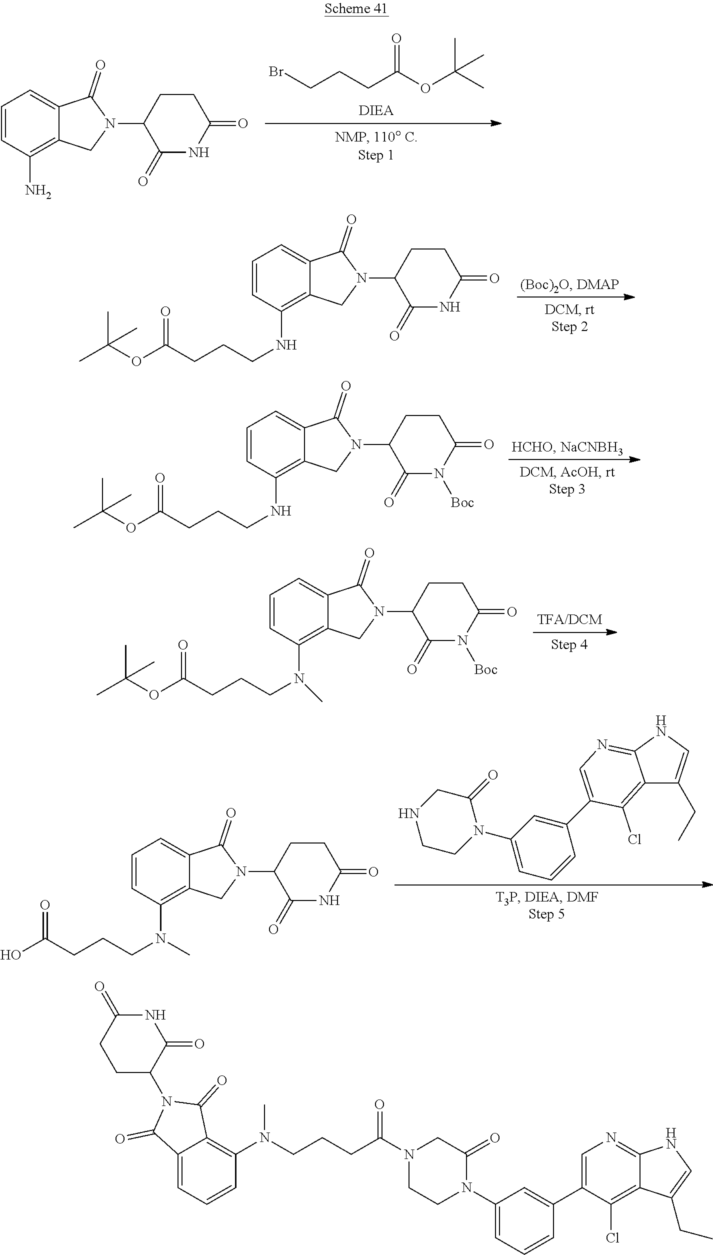

Step 1. Preparation of tert-butyl 4-((2-(2,6-dioxopiperidin-3-yl)-1-oxoisoindolin-4-yl)amino) butanoate: Following General step G1, the product tert-butyl 4-((2-(2,6-dioxopiperidin-3-yl)-1-oxoisoindolin-4-yl)amino) butanoate was obtained as yellow solid (1.1 g, 47%). Mass (m/z): 401.8 [M+H]⁺.

Step 2. Preparation of tert-butyl 3-(4-((4-(tert-butoxy)-4-oxobutyl)amino)-1-oxoisoindolin-2-yl)-2,6-dioxopiperidine-1-carboxylate: Following General step H, the product tert-butyl 3-(4-((4-(tert-butoxy)-4-oxobutyl)amino)-1-oxoisoindolin-2-yl)-2,6-dioxopiperidine-1-carboxylate was obtained as yellow oil (654 mg, 47%). Mass (m/z): 523.9 [M+Na]⁺.

Step 3. Preparation of tert-butyl 3-(4-((4-(tert-butoxy)-4-oxobutyl)(methyl)amino)-1-oxoisoindolin-2-yl)-2,6-dioxopiperidine-1-carboxylate: Following General step I, the product tert-butyl 3-(4-((4-(tert-butoxy)-4-oxobutyl)(methyl)amino)-1-oxoisoindolin-2-yl)-2,6-dioxopiperidine-1-carboxylate was obtained as yellow oil (272 mg, 88%). Mass (m/z): 515.8 [M+H]⁺.

Step 4. Preparation of 4-((2-(2,6-dioxopiperidin-3-yl)-1-oxoisoindolin-4-yl)(methyl)amino) butanoic acid: Following General step B2, the product 4-((2-(2,6-dioxopiperidin-3-yl)-1-oxoisoindolin-4-yl)(methyl)amino) butanoic acid was obtained as yellow oil (310 mg, 79%). Mass (m/z): 359.9 [M+H]⁺.

Step 5. Preparation of 3-(4-((4-(4-(3-(4-chloro-3-ethyl-1H-pyrrolo[2,3-b]pyridin-5-yl)phenyl)-3-oxopiperazin-1-yl)-4-oxobutyl)(methyl)amino)-1-oxoisoindolin-2-yl)piperidine-2,6-dione: Following General step F, the product was obtained as white solid (48 mg, 8%). Mass (m/z): 696.7 [M+H]⁺. ¹H NMR (400 MHZ, CD₃OD) δ 8.13 (s, 1H), 7.58-7.53 (m, 1H), 7.48-7.44 (m, 2H), 7.41 (dd, J=14.0, 5.8 Hz, 2H), 7.33-7.27 (m, 2H), 7.15 (d, J=8.2 Hz, 1H), 5.21-5.13 (m, 1H), 4.60 (dd, J=21.8, 11.2 Hz, 2H), 4.34-4.23 (m, 2H), 4.12 (q, J=7.2 Hz, 1H), 3.79 (s, 3H), 3.02 (q, J=7.4 Hz, 2H), 2.97 (s, 3H), 2.93-2.86 (m, 1H), 2.82 (s, 1H), 2.59 (d, J=13.0 Hz, 1H), 2.49 (t, J=6.8 Hz, 2H), 2.21 (dd, J=9.8, 5.2 Hz, 1H), 2.04 (d, J=8.8 Hz, 2H), 1.95 (dd, J=13.8, 6.8 Hz, 2H), 1.36 (d, J=7.4 Hz, 3H).

### Example 23: 3-ethyl-1H-pyrrolo 5-((6-(4-(3-(4-chloro-3-ethyl-1H-pyrrolo[2,3-b]pyridin-5-yl)phenyl)-3-oxopiperazin-1-yl)-6-oxohexyl)amino)-2-(2,6-dioxopiperidin-3-yl)isoindoline-1,3-dione

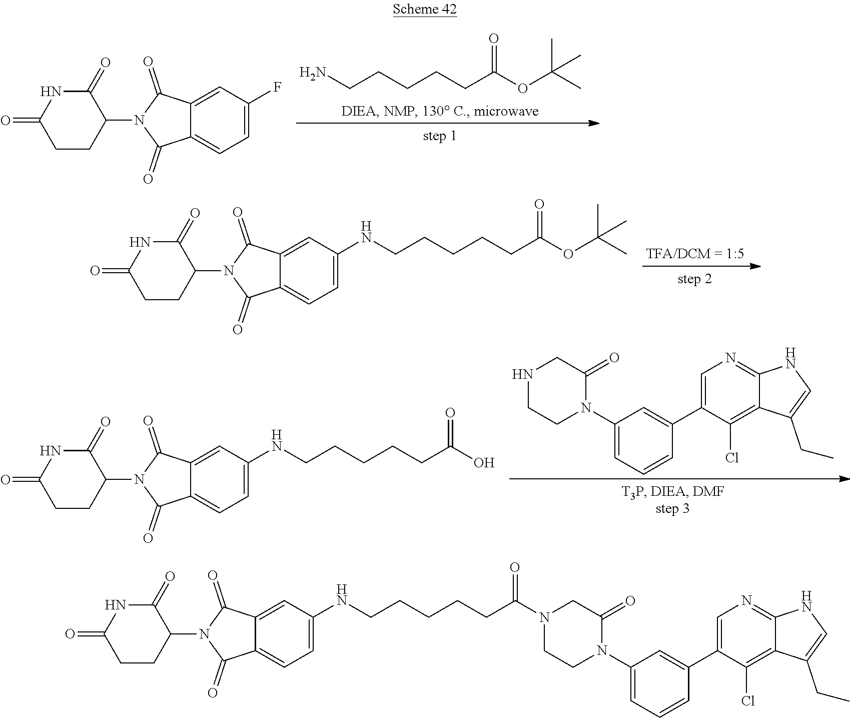

Step 1. Preparation of tert-butyl 6-{[2-(2,6-dioxopiperidin-3-yl)-1,3-dioxoisoindol-5-yl]amino}hexanoate: To a solution of 2-(2,6-dioxopiperidin-3-yl)-5-fluoroisoindole-1,3-dione (1.97 g, 7.1 mmol) and tert-butyl 6-aminohexanoate (1.46 g, 7.8 mmol) in NMP (20 mL) was added DIEA (2.75 mg, 0.02 mol). The reaction was placed in a microwave synthesizer at 130° C. for 50 mins. Water (100 mL) was added and the mixture was extracted with EA (150 mL×3). The combined organic layers were washed with brine (50 mL×2), dried over Na₂SO₄. Then by filtration, the filtrate was concentrated. The crude product was purified by Flash Chromatography (PE/EA=3:1) to give the product as a yellow solid (600 mg, 15%). Mass (m/z): 465.9 [M+Na]⁺.

Step 2. Preparation of 6-{[2-(2,6-dioxopiperidin-3-yl)-1,3-dioxoisoindol-5-yl]amino}hexanoic acid: Following General step B2, the product was obtained as a yellow oil (180 mg, 62%). Mass (m/z): 387.8 [M+H]⁺.

Step 3. Preparation of 5-({6-[4-(3-{4-chloro-3-ethyl-1H-pyrrolo[2,3-b]pyridin-3-yl}phenyl)-3-oxopiperazin-1-yl]-6-oxohexyl}amino)-2-(2,6-dioxopiperidin-3-yl) isoindole-1,3-dione: Following General step F, the product was obtained s a light yellow solid (23 mg, 6%). Mass (m/z): 723.6 [M+H]⁺. ¹H NMR (400 MHZ, DMSO-d₆) δ 11.80 (d, J=1.8 Hz, 1H), 11.06 (s, 1H), 8.12 (s, 1H), 7.60-7.45 (m, 3H), 7.40 (t, J=8.6 Hz, 3H), 7.24-7.01 (m, 1H), 6.94 (s, 1H), 6.84 (dd, J=8.4, 1.6 Hz, 1H), 5.02 (dd, J=12.9, 5.3 Hz, 1H), 4.29 (s, 1H), 4.19 (s, 1H), 3.85 (s, 2H), 3.83-3.74 (m, 2H), 3.53 (d, J=10.7 Hz, 4H), 3.16 (dd, J=7.3, 5.9 Hz, 2H), 2.92 (q, J=7.4 Hz, 2H), 2.40 (dd, J=15.4, 7.6 Hz, 2H), 1.58 (dt, J=16.0, 8.1 Hz, 4H), 1.46-1.36 (m, 2H), 1.27 (t, J=7.4 Hz, 3H).

### Example 24: 3-ethyl-1H-pyrrolo 5-(4-(3-(4-(3-(4-chloro-3-ethyl-1H-pyrrolo[2,3-b]pyridin-5-yl)phenyl)-3-oxopiperazin-1-yl) propanoyl)piperazin-1-yl)-2-(2,6-dioxopiperidin-3-yl)isoindoline-1,3-dione

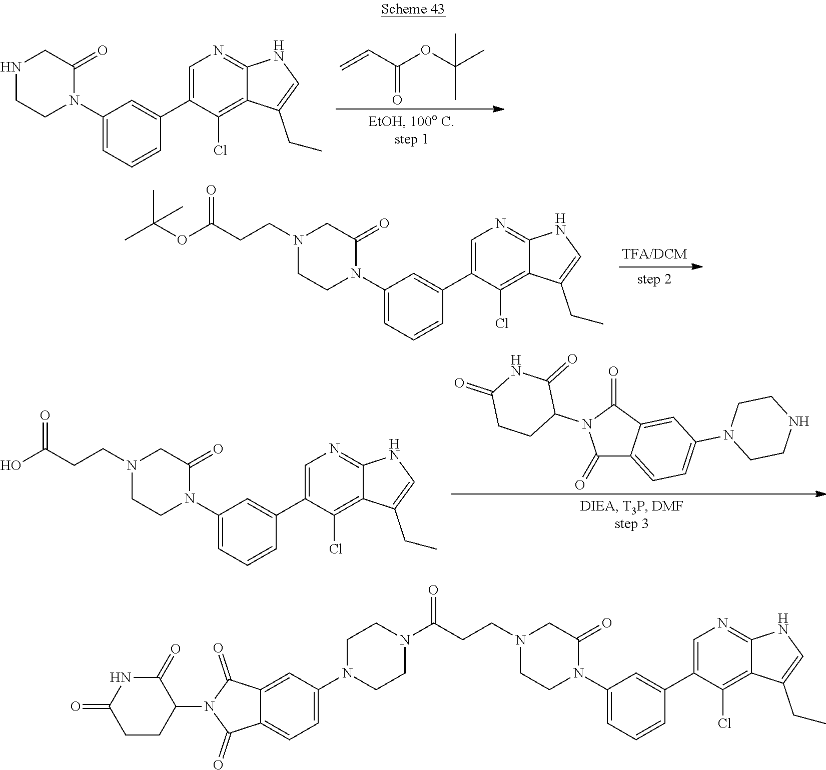

Step 1. Preparation of tert-butyl (4-{3-[4-chloro-3-ethyl-1H-pyrrolo[2,3-b]pyridin-3-yl]phenyl}-3-oxopiperazin-1-yl) formate: To a mixture of 1-(3-{4-chloro-3-ethyl-1H-pyrrolo[2,3-b]pyridin-3-yl}phenyl)piperazin-2-one (300 mg, 0.85 mmol), tert-butyl prop-2-enoate (140.88 mg, 1.1 mmol) and DIEA (218.54 mg, 1.69 mmol) in EtOH (3.5 mL) under N₂ stirred at 100° C. for 36 hrs. The reaction was filtered and concentrated. The residue was purified by combi-flash with DCM/MeOH (20:1) to afford product (260 mg, yield: 39%) as a brown solid. Mass (m/z): 482.8 [M+H]⁺.

Step 2. Preparation of 3-[4-(3-{4-chloro-3-ethyl-1H-pyrrolo[2,3-b]pyridin-3-yl}phenyl)-3-oxopiperazin-1-yl]propanoic acid: Following General step B2, the crude product was obtained as a yellowish solid (200 mg, 72%). Mass (m/z): 426.7 [M+H]⁺.

Step 3. Preparation of 5-(4-{3-[4-(3-{4-chloro-3-ethyl-1H-pyrrolo[2,3-b]pyridin-3-yl}phenyl)-3-oxopiperazin-1-yl]propanoyl}piperazin-1-yl)-2-(2,6-dioxopiperidin-3-yl) isoindole-1,3-dione: Following General step F, the product was obtained as a light yellow solid compound (50 mg, 14%). Mass (m/z): 750.6 [M+H]⁺. ¹H NMR (400 MHZ, DMSO-d₆) δ 11.80 (s, 1H), 11.09 (s, 1H), 8.12 (s, 1H), 7.70 (d, J=8.5 Hz, 1H), 7.50 (t, J=7.8 Hz, 1H), 7.45 (s, 1H), 7.42-7.34 (m, 4H), 7.26 (d, J=8.6 Hz, 1H), 5.08 (dd, J=12.9, 5.3 Hz, 1H), 3.75-3.70 (m, 2H), 3.67 (s, 2H), 3.63 (s, 2H), 3.50 (d, J=28.6 Hz, 6H), 3.25 (s, 2H), 2.96-2.83 (m, 5H), 2.73-2.61 (m, 5H), 1.27 (t, J=7.4 Hz, 3H).

### Example 25: 3-ethyl-1H-pyrrolo 5-(4-(3-(4-(3-(4-chloro-3-ethyl-1H-pyrrolo[2,3-b]pyridin-5-yl)phenyl)-3-oxopiperazin-1-yl) propyl)piperazin-1-yl)-2-(2,6-dioxopiperidin-3-yl)-6-fluoroisoindoline-1,3-dione

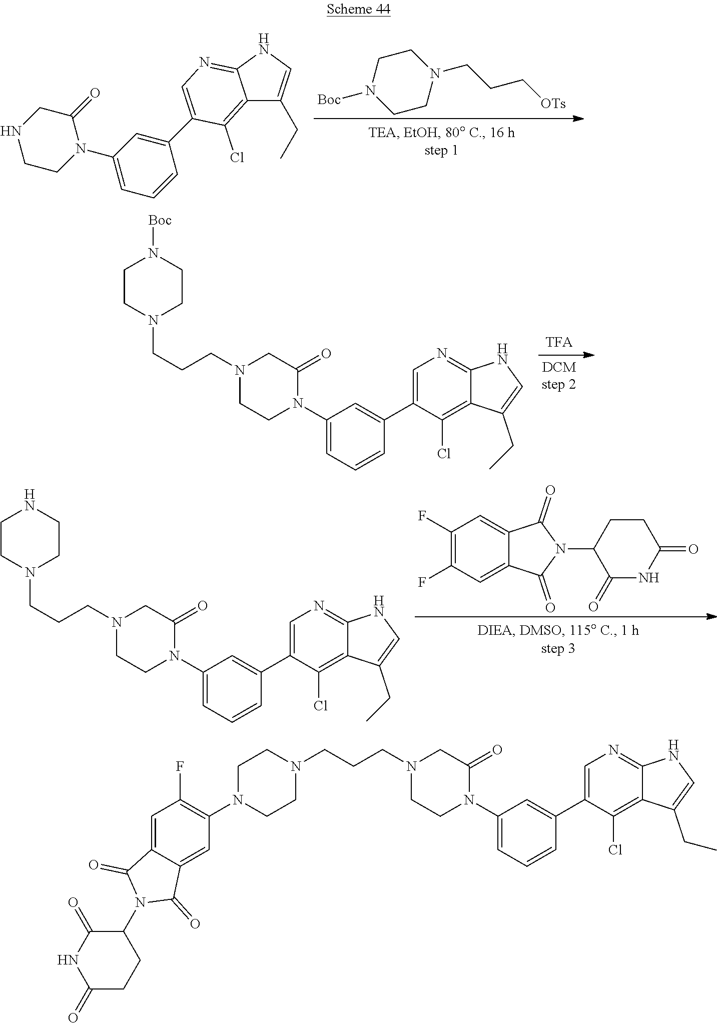

Step 1. (General Step G2) Preparation of tert-butyl 4-(3-(4-(3-(4-chloro-3-ethyl-1H-pyrrolo[2,3-b]pyridin-5-yl)phenyl)-3-oxopiperazin-1-yl) propyl)piperazine-1-carboxylate: To a solution of 1-(3-(4-chloro-3-ethyl-1H-pyrrolo[2,3-b]pyridin-5-yl)phenyl)piperazin-2-one (300 mg, 0.845 mmol) and TEA (256 mg, 2.536 mmol) in EtOH (10 mL) was added tert-butyl 4-(3-(tosyloxy) propyl)piperazine-1-carboxylate (371 mg, 0.93 mmol). The reaction mixture was stirred at 80° C. under N₂ for 16 hrs. The mixture was concentrated and the residue was purified by flash column (DCM/MeOH=10:1) to give the product as a yellow solid (280 mg, 56%). Mass (m/z): 580.9 [M+H]⁺.

Step 2. Preparation of 1-(3-(4-chloro-3-ethyl-1H-pyrrolo[2,3-b]pyridin-5-yl)phenyl)-4-(3-(piperazin-1-yl) propyl)piperazin-2-one: Following General step B2, the product was obtained as a brown solid (300 mg, purity: 70%). Mass (m/z): 481.1 [M+H]⁺.

Step 3. Preparation of 5-(4-{3-[4-(3-{4-chloro-3-ethyl-1H-pyrrolo[2,3-b]pyridin-3-yl}phenyl)-3-oxopiperazin-1-yl]propyl}piperazin-1-yl)-2-(2,6-dioxopiperidin-3-yl)-6-fluoroisoindole-1,3-dione: Following General step L, the product was obtained as a yellow solid (150 mg, 74%). Mass (m/z): 755.0 [M+H]⁺. ¹H NMR (400 MHz, DMSO-d₆) δ 11.80 (d, J=2.2 Hz, 1H), 11.12 (s, 1H), 8.17 (s, 1H), 8.12 (s, 1H), 7.73 (d, J=11.4 Hz, 1H), 7.50 (t, J=7.8 Hz, 1H), 7.47-7.45 (m, 1H), 7.39 (dd, J=12.2, 9.2 Hz, 3H), 5.11 (dd, J=12.8, 5.4 Hz, 1H), 3.77-3.70 (m, 2H), 3.25 (s, 4H), 3.19 (s, 2H), 2.92 (dd, J=14.6, 7.2 Hz, 3H), 2.82-2.78 (m, 2H), 2.61-2.50 (m, 6H), 2.49-2.35 (m, 4H), 2.06-2.00 (m, 1H), 1.74-1.63 (m, 2H), 1.28 (t, J=7.4 Hz, 3H).

### Example 26: 3-ethyl-1H-pyrrolo 5-(4-(2-(4-(3-(4-chloro-3-ethyl-1H-pyrrolo[2,3-b]pyridin-5-yl)phenyl)-3-oxopiperazin-1-yl)ethoxy)piperidin-1-yl)-2-(2,6-dioxopiperidin-3-yl)-6-fluoroisoindoline-1,3-dione

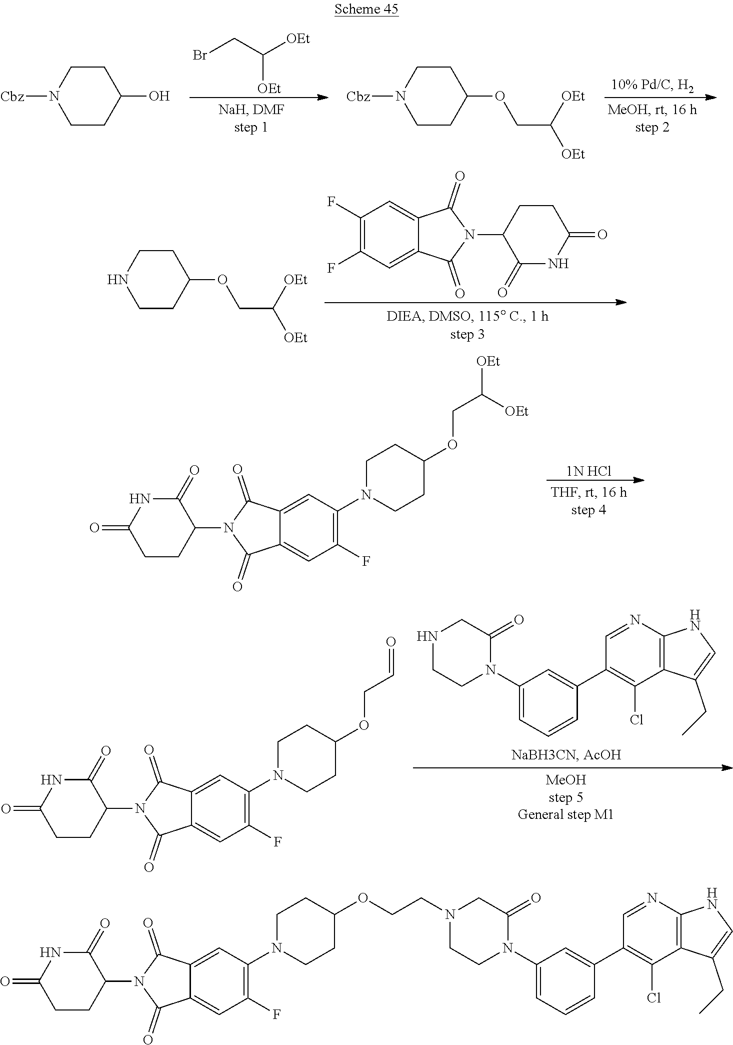

Step 1. Preparation of benzyl 4-(2,2-diethoxyethoxy)piperidine-1-carboxylate: From compound benzyl 4-hydroxypiperidine-1-carboxylate (500 mg, 2.12 mmol), following General step J1, the desired product (147 mg, 19%) was obtained as light-yellow oil. Mass (m/z): 325.5 [M+H]⁺.

Step 2. Preparation of 4-(2,2-diethoxyethoxy)piperidine: From benzyl 4-(2,2-diethoxyethoxy)piperidine-1-carboxylate (147 mg, 0.41 mmol), following General step K, the product (118 mg, 98%) was obtained as yellow oil. Mass (m/z): 218.3 [M+H]⁺.

Step 3. Preparation of 5-(4-(2,2-diethoxyethoxy)piperidin-1-yl)-2-(2,6-dioxopiperidin-3-yl)-6-fluoroisoindoline-1,3-dione: From 4-(2,2-diethoxyethoxy)piperidine (118 mg, 0.543 mmol), following General step L, the product (125 mg, 45%) was obtained as yellow oil. Mass (m/z): 492.2 [M+H]⁺.

Step 4. Preparation of 2-((1-(2-(2,6-dioxopiperidin-3-yl)-6-fluoro-1,3-dioxoisoindolin-5-yl)piperidin-4-yl)oxy) acetaldehyde: A solution of 5-(4-(2,2-diethoxyethoxy)piperidin-1-yl)-2-(2,6-dioxopiperidin-3-yl)-6-fluoroisoindoline-1,3-dione (125 mg, 0.254 mmol) in 1N

HCl/THF (5 mL) was stirred at 25° C. for 16 hours. The reaction was concentrated under vacuum and water (10 mL) was added. The mixture was adjusted to pH 7 with 1 N aqueous NaOH, extracted with EA (10 mL×3). The organic layer was washed with brine (10 mL), dried over anhydrous sodium sulfate and concentrated. The residue was purified by Flash Chromatography (PE/EA=4:1) to give the product (61 mg, 57%) as yellow oil. Mass (m/z): 418.3 [M+H]⁺.

Step 5. (General Step M1) Preparation of 5-(4-{2-[4-(3-{4-chloro-3-ethyl-1H-pyrrolo[2,3-b]pyridin-3-yl}phenyl)-3-oxopiperazin-1-yl]ethoxy}piperidin-1-yl)-2-(2,6-dioxopiperidin-3-yl)-6-fluoroisoindole-1,3-dione: To a solution of 2-((1-(2-(2,6-dioxopiperidin-3-yl)-6-fluoro-1,3-dioxoisoindolin-5-yl)piperidin-4-yl)oxy) acetaldehyde (61 mg, 0.146 mmol) in MeOH/HOAc (10:1, 5.5 mL) was added intermediate A (52 mg, 0.146 mmol) and sodium cyanoborohydride (18 mg, 0.292 mmol). The reaction mixture was stirred at rt for 2 hrs. The reaction mixture was quenched with water (10 mL) and extracted with EA (10 mL×3). The organic layers were combined, washed with saturated aqueous sodium chloride solution (10 mL) and dried over anhydrous sodium sulfate. Then by filtration, the filtrate was concentrated. The residue was purified by Prep-HPLC [Gemini-C₁₈, 150×21.2 mm, 5 um; ACN-H₂O (0.1% FA), 30-45] to give the product (40 mg, 36%) as a yellow solid. Mass (m/z): 757.3 [M+H]⁺. ¹H NMR (400 Hz, CD₃OD): δ 8.11 (s, 1H), 7.59-7.33 (m, 6H), 7.26 (s, 1H), 5.07 (s, 1H), 4.61 (s, 1H), 3.79 (dt, J=24.8 Hz, 4H), 3.61 (s, 1H), 3.53 (s, 2H), 3.47 (s, 2H), 3.04 (m, 6H), 2.78 (m, 5H), 2.08 (s, 3H), 1.78 (m, 2H), 1.32 (m, 4H).

### Example 27: 3-ethyl-1H-pyrrolo 5-(4-(3-(4-(3-(4-chloro-3-ethyl-1H-pyrrolo[2,3-b]pyridin-5-yl)phenyl)-3-oxopiperazin-1-yl) propyl)piperazin-1-yl)-2-(2,6-dioxopiperidin-3-yl)isoindoline-1,3-dione

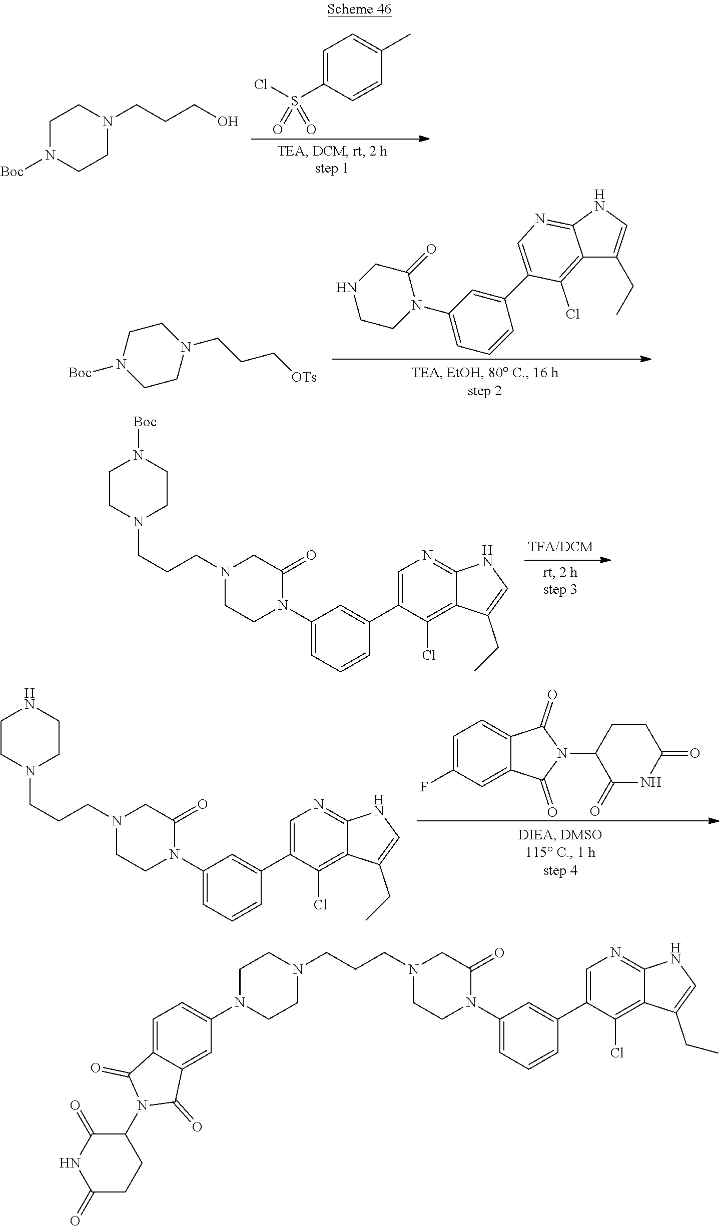

Step 1. Preparation of tert-butyl [4-(3-{[(4-methylbenzene) sulfonyl]oxy}propyl) piperazin-1-yl]formate: To a mixture of tert-butyl [4-(3-hydroxypropyl)piperazin-1-yl]formate (2.0 g, 8.20 mmol) and TEA (2.82 g, 27.8 mmol) in DCM (40 mL) was added 4-methylbenzenesulfonyl chloride (2.66 g, 13.9 mmol). The reaction was stirred at rt for 2 hrs. The reaction mixture was concentrated under reduced pressure to give the desired product tert-butyl [4-(3-{[(4-methylbenzene) sulfonyl]oxy}propyl)piperazin-1-yl]formate as yellow oil (1.97 g, 47%). Mass (m/z): 398.9 [M+H]⁺.

Step 2. Preparation of tert-butyl (4-{3-[4-(3-{4-chloro-3-ethyl-1H-pyrrolo[2,3-b] pyridin-3-yl}phenyl)-3-oxopiperazin-1-yl]propyl}piperazin-1-yl) formate: Following General step G2, the product tert-butyl (4-{3-[4-(3-{4-chloro-3-ethyl-1H-pyrrolo[2,3-b]pyridin-3-yl}phenyl)-3-oxo piperazin-1-yl]propyl}piperazin-1-yl) formate was obtained as pale yellow solid (440 mg, 75%). Mass (m/z): 580.8 [M+H]⁺.

Step 3. Preparation of 1-(3-{4-chloro-3-ethyl-1H-pyrrolo[2,3-b]pyridin-3-yl}phenyl)-4-[3-(piperazin-1-yl) propyl]piperazin-2-one: Following General step B2, the product 1-(3-{4-chloro-5-ethyl-7H-pyrrolo[2,3-b]pyridin-3-yl}phenyl)-4-[3-(piperazin-1-yl) propyl]piperazin-2-one was obtained as brown solid (470 mg, 90%). Mass (m/z): 481.0 [M+H]⁺.

Step 4. Preparation of 5-(4-{3-[4-(3-{4-chloro-3-ethyl-1H-pyrrolo[2,3-b]pyridin-3-yl}phenyl)-3-oxopiperazin-1-yl]propyl}piperazin-1-yl)-2-(2,6-dioxopiperidin-3-yl) isoindole-1,3-dione: Following General step L, the product 5-(4-{3-[4-(3-{4-chloro-3-ethyl-1H-pyrrolo[2,3-b]pyridin-3-yl}phenyl)-3-oxo piperazin-1-yl]propyl}piperazin-1-yl)-2-(2,6-dioxopiperidin-3-yl) isoindole-1,3-dione was obtained as yellow solid (10 mg, 8%). Mass (m/z): 736.7 [M+H]⁺. ¹H NMR (400 MHZ, CD₃OD) δ 8.10 (s, 1H), 7.72 (d, J=8.5 Hz, 1H), 7.56 (t, J=7.8 Hz, 1H), 7.45 (dd, J=9.0, 1.6 Hz, 3H), 7.38 (dd, J=8.0, 1.2 Hz, 1H), 7.31 (dd, J=8.4, 2.4 Hz, 1H), 7.27 (s, 1H), 5.09 (dd, J=12.6, 5.6 Hz, 1H), 3.84-3.82 (m, 2H), 3.66 (s, 4H), 3.37 (s, 2H), 3.21 (s, 4H), 3.09-3.05 (m, 2H), 3.00 (d, J=4.0 Hz, 1H), 2.96 (t, J=5.4 Hz, 2H), 2.88-2.82 (m, 1H), 2.78-2.76 (m, 1H), 2.74-2.72 (m, 1H), 2.66 (dd, J=8.8, 4.4 Hz, 2H), 2.16-2.09 (m, 1H), 2.01-1.94 (m, 2H), 1.33 (t, J=7.4 Hz, 3H).

### Example 28: 5-(4-(2-(4-(3-(4-chloro-3-ethyl-1H-pyrrolo[2,3-b]pyridin-5-yl)phenyl)-3-oxopiperazin-1-yl)ethyl)piperazin-1-yl)-2-(2,6-dioxopiperidin-3-yl)isoindoline-1,3-dione

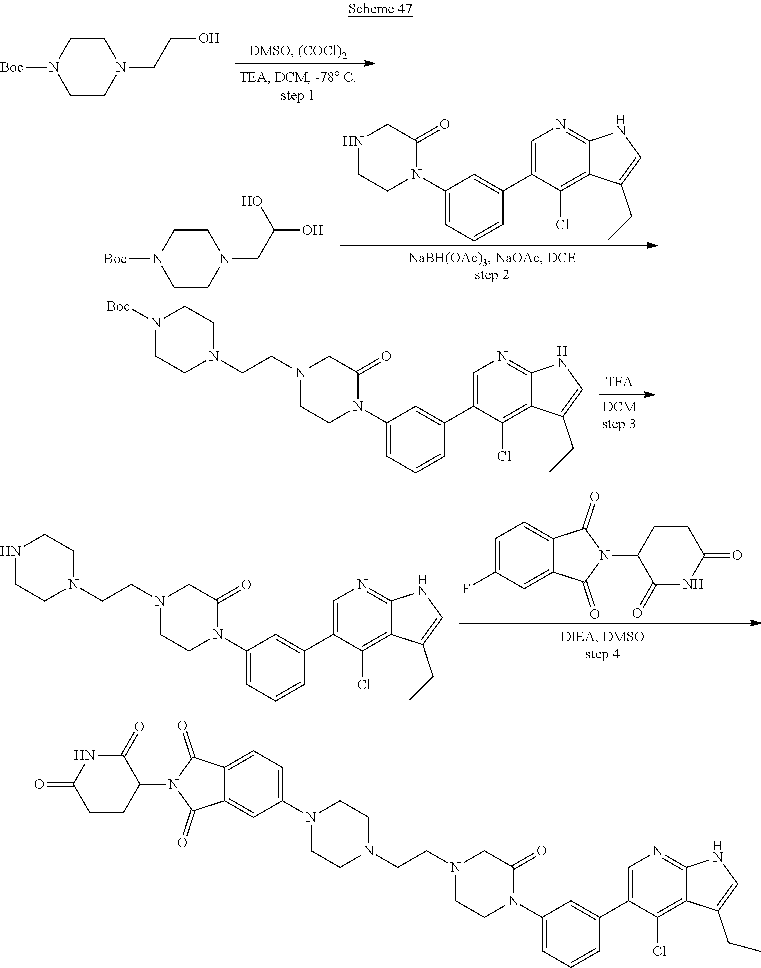

Step 1. Preparation of tert-butyl 4-(2,2-dihydroxyethyl)piperazine-1-carboxylate: To a stirred solution of oxalyl chloride (5.49 g, 43.2 mmol) in DCM (90 mL) at −78° C. was added DMSO (6.74 g, 86.4 mmol). The reaction mixture was stirred for 10 min followed by addition of tert-butyl 4-(2-hydroxyethyl)piperazine-1-carboxylate (5 g, 21.6 mmol, dissolved in 10 mL of DCM). The reaction mixture was stirred at the same temperature for 15 min. Then TEA (13.09 g, 129.6 mmol) was added and continued to stir for another 1 hour while allowing the reaction mixture to reach at room temperature. Water (100 mL) was added and the mixture was extracted with DCM (100 mL×3). The combined organic layers were washed with brine (100 mL×2) dried over Na₂SO₄. Then by filtration, the filtrate was concentrated to dryness to give product as brown oil (5.8 g, purity: 80%). Mass (m/z): 247.1 [M+H]⁺.

Step 2. Preparation of tert-butyl 4-(2-(4-(3-(4-chloro-3-ethyl-1H-pyrrolo[2,3-b]pyridin-5-yl)phenyl)-3-oxopiperazin-1-yl)ethyl)piperazine-1-carboxylate: A solution of tert-butyl 4-(2,2-dihydroxyethyl)piperazine-1-carboxylate (200 mg, 0.542 mmol), 1-(3-(4-chloro-3-ethyl-1H-pyrrolo[2,3-b]pyridin-5-yl)phenyl)piperazin-2-one (201 mg, 0.81 mmol), NaOAc (133 mg, 1.63 mmol) and NaBH(OAc)₃, (344 mg, 1.63 mmol) in DCE (10 mL) was stirred at rt for 3 hrs. The mixture was concentrated and the residue was purified by flash column (DCM/MeOH=20:1) to give the product tert-butyl 4-(2-(4-(3-(4-chloro-3-ethyl-1H-pyrrolo[2,3-b]pyridin-5-yl)phenyl)-3-oxopiperazin-1-yl)ethyl)piperazine-1-carboxylate (140 mg, 31%). Mass (m/z): 566.9 [M+H]⁺.

Step 3. Preparation of 1-(3-(4-chloro-3-ethyl-1H-pyrrolo[2,3-b]pyridin-5-yl)phenyl)-4-(2-(piperazin-1-yl)ethyl)piperazin-2-one: Following General step B2, the product was obtained as a yellow solid (220 mg, purity: 50%). Mass (m/z): 469.9 [M+H]⁺.

Step 4. 5-(4-(2-(4-(3-(4-chloro-3-ethyl-1H-pyrrolo[2,3-b]pyridin-5-yl)phenyl)-3-oxopiperazin-1-yl)ethyl)piperazin-1-yl)-2-(2,6-dioxopiperidin-3-yl)isoindoline-1,3-dione: Following General step L, the product was obtained as a yellow solid (160 mg, 74%). Mass (m/z): 722.7 [M+H]⁺. ¹H NMR (400 MHZ, CDCl₃) δ 11.76 (d, J=2.0 Hz, 1H), 11.06 (s, 1H), 8.09 (s, 1H), 7.73-7.64 (m, 1H), 7.47 (t, J=7.8 Hz, 1H), 7.43 (d, J=1.8 Hz, 1H), 7.36 (q, J=8.4 Hz, 4H), 7.27 (s, 1H), 5.04 (dd, J=13.0, 5.0 Hz, 1H), 3.72 (s, 2H), 3.50-3.37 (m, 4H), 2.93-2.80 (m, 6H), 2.66-2.52 (m, 4H), 2.50 (s, 3H), 2.44-2.35 (m, 2H), 2.04-1.89 (m, 2H), 1.24 (t, J=7.4 Hz, 3H).

### Example 29: 5-(4-(2-(4-(3-(4-chloro-3-ethyl-1H-pyrrolo[2,3-b]pyridin-5-yl)phenyl)-3-oxopiperazin-1-yl) acetyl)piperazin-1-yl)-2-(2,6-dioxopiperidin-3-yl)-6-fluoroisoindoline-1,3-dione

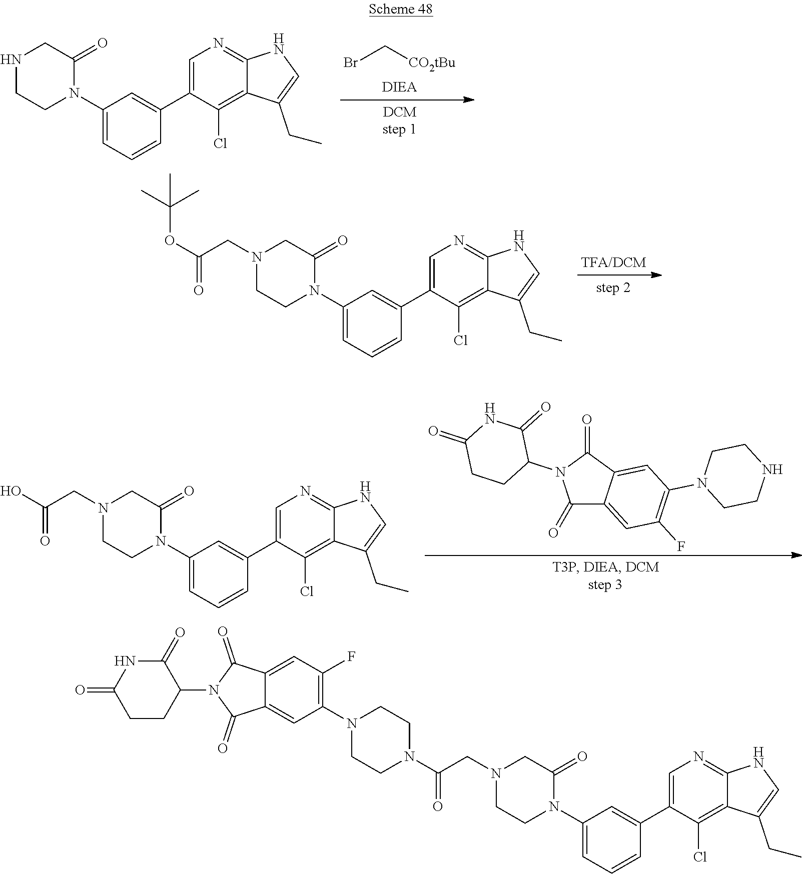

Step 1. Preparation of tert-butyl 2-[4-(3-{4-chloro-3-ethyl-1H-pyrrolo[2,3-b]pyridin-3-yl}phenyl)-3-oxopiperazin-1-yl]acetate: A solution of 1-(3-(4-chloro-3-ethyl-1H-pyrrolo[2,3-b]pyridin-5-yl)phenyl)piperazin-2-one (90 mg, 0.25 mmol), tert-butyl 2-bromoacetate (49 mg, 0.25 mmol) and DIEA (65 mg, 0.50 mmol) in DCM (5 mL). The resulting mixture was stirred at 25° C. under N₂ for 3 hrs. The reaction was concentrated and the residue was purified by silica gel column chromatography (PE/EA=2:1) to give the product (60 mg, 31%) as a yellow solid. Mass (m/z): 469 [M+H]⁺.

Step 2. Preparation of 2-(4-(3-(4-chloro-3-ethyl-1H-pyrrolo[2,3-b]pyridin-5-yl)phenyl)-3-oxopiperazin-1-yl) acetic acid: Following General step B2, the product (50 mg, 76%) was obtained as yellow oil. Mass (m/z): 413 [M+H]⁺.

Step 3. Preparation of 5-(4-(2-(4-(3-(4-chloro-3-ethyl-1H-pyrrolo[2,3-b]pyridin-5-yl)phenyl)-3-oxopiperazin-1-yl) acetyl)piperazin-1-yl)-2-(2,6-dioxopiperidin-3-yl)-6-fluoroisoindoline-1,3-dione: Following General step F, the product (13 mg 28%) was obtained as a yellow solid. Mass (m/z): 755 [M+H]⁺. ¹HNMR (400 MHZ, CD₃OD) δ 9.71 (s, 1H), 9.00 (s, 1H), 8.16 (s, 1H), 7.52-7.40 (m, 2H), 7.38-7.12 (m, 4H), 7.00 (s, 1H), 4.96-4.92 (m, 1H), 3.82-3.40 (m, 6H), 3.27 (s, 2H), 3.14 (s, 2H), 3.02-2.92 (m, 4H), 2.89-2.80 (m, 4H), 2.70-2.78 (m, 3H), 2.16-2.08 (m, 1H), 1.34-1.25 (m, 3H).

### Example 30: 5-(4-(3-(4-(3-(4-chloro-3-ethyl-1H-pyrrolo[2,3-b]pyridin-5-yl)phenyl)-3-oxopiperazin-1-yl) propanoyl)piperazin-1-yl)-2-(2,6-dioxopiperidin-3-yl)-6-fluoroisoindoline-1,3-dione

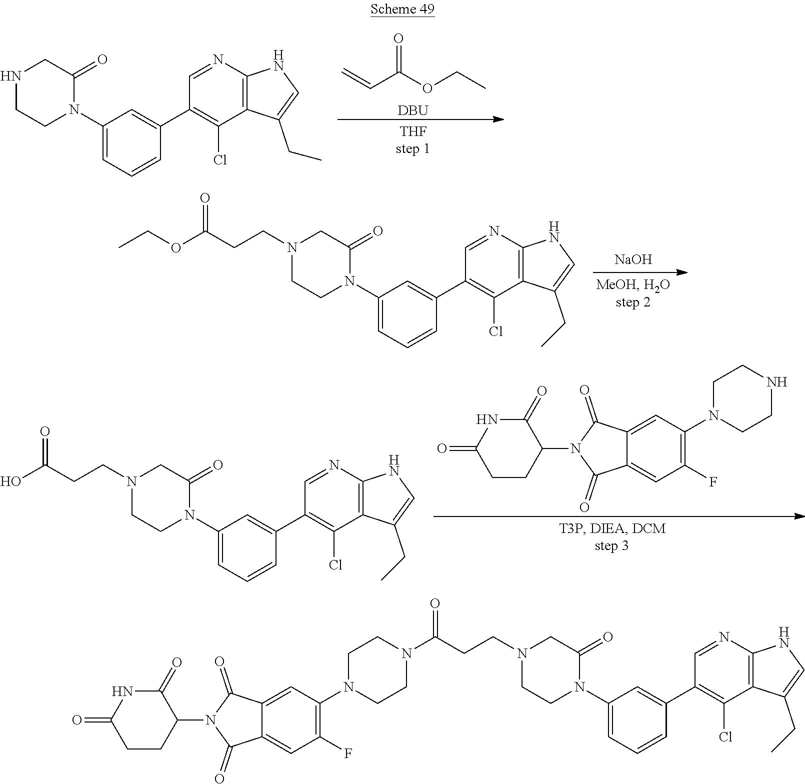

Step 1. Preparation of ethyl 3-[4-(3-{4-chloro-3-ethyl-1H-pyrrolo[2,3-b]pyridin-3-yl}phenyl)-3-oxopiperazin-1-yl]propanoate: To a solution of 1-(3-(4-chloro-3-ethyl-1H-pyrrolo[2,3-b]pyridin-5-yl)phenyl)piperazin-2-one (50 mg, 0.14 mmol) and ethyl prop-2-enoate (140 mg, 0.14 mmol) in THF (5 mL) was added DBU (21 mg, 0.14 mmol). The resulting mixture was stirred for 1 hour at 25° C. The result was concentrated under reduced pressure and the residue was purified by silica gel column chromatography (PE/EA=5:1) to give the product (30 mg, 24%) as a yellow solid. Mass (m/z): 455 [M+H]⁺.

Step 2. Preparation of 3-[4-(3-{4-chloro-3-ethyl-1H-pyrrolo[2,3-b]pyridin-3-yl}phenyl)-3-oxopiperazin-1-yl]propanoic acid: To a solution of ethyl 3-[4-(3-{4-chloro-3-ethyl-1H-pyrrolo[2,3-b]pyridin-3-yl}phenyl)-3-oxopiperazin-1-yl]propanoate (30 mg, 0.065 mmol) in MeOH (3 mL) and H₂O (1 mL) was added NaOH (26 mg, 0.65 mmol). The resulting mixture was stirred for 1 hour at 25° C. MeOH was removed and water (10 mL) was added. The mixture was adjusted pH 7 with aq. HCl (1 N), and then extracted with EA (10 mL×3). The combined organic layer was washed with brine (20 mL×2), then dried over anhydrous Na₂SO₄. After filtration, the solution was concentrated under vacuum to give the product (25 mg 85%) as a yellow solid. Mass (m/z): 426 [M+H]⁺.

Step 3. Preparation of 5-(4-(3-(4-(3-(4-chloro-3-ethyl-1H-pyrrolo[2,3-b]pyridin-5-yl)phenyl)-3-oxopiperazin-1-yl) propanoyl)piperazin-1-yl)-2-(2,6-dioxopiperidin-3-yl)-6-fluoroisoindoline-1,3-dione: Following General step F, the product (12 mg 21%) was obtained as a yellow solid. Mass (m/z): 769 [M+H]⁺. ¹HNMR (400 MHZ, CD₃OD) δ 9.73 (s, 1H), 9.08 (s, 1H), 8.16 (s, 1H), 7.52-7.49 (m, 2H), 7.47-7.35 (m, 4H), 7.12 (s, 1H), 4.97-4.93 (m, 1H), 3.83-3.76 (m, 4H), 3.69 (s, 2H), 3.41 (s, 2H), 3.27-3.27 (m, 4H), 3.02-2.75 (m, 7H), 2.71-2.67 (m, 2H), 2.65-2.62 (t, J=4.8 Hz, 2H), 2.18 (s, 1H), 1.34-1.25 (m, 3H).

### Example 31: 3-ethyl-1H-pyrrolo 5-(4-(2-(4-(3-(4-chloro-3-cyclopropyl-1H-pyrrolo[2,3-b]pyridin-5-yl)phenyl)-3-oxopiperazin-1-yl)-2-oxoethoxy)piperidin-1-yl)-2-(2,6-dioxopiperidin-3-yl)isoindoline-1,3-dione

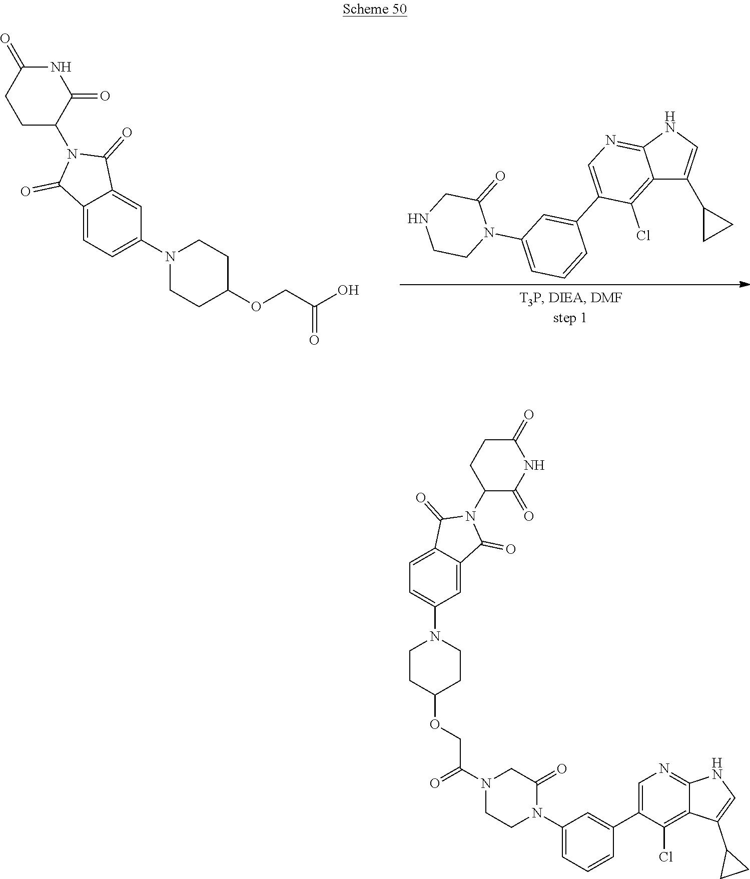 [SMILES: O=C(O)COC1CCN(c2ccc3c(c2)C(=O)N(C2CCC(=O)NC2=O)C3=O)CC1 ; O=C1CNCCN1c1cccc(-c2cnc3[nH]cc(C4CC4)c3c2Cl)c1 ; O=C1CCC(N2C(=O)c3ccc(N4CCC(OCC(=O)N5CCN(c6cccc(-c7cnc8[nH]cc(C9CC9)c8c7Cl)c6)C(=O)C5)CC4)cc3C2=O)C(=O)N1]

Step 1. Preparation of 5-(4-{2-[4-(3-{4-chloro-3-ethyl-1H-pyrrolo[2,3-b]pyridin-3-yl}phenyl)-3-oxopiperazin-1-yl]-2-oxoethoxy}piperidin-1-yl)-2-(2,6-dioxopiperidin-3-yl) isoindole-1,3-dione: Following General step F, the desired product 5-(4-{2-[4-(3-{4-chloro-3-ethyl-1H-pyrrolo[2,3-b]pyridin-3-yl}phenyl)-3-oxopiperazin-1-yl]-2-oxoethoxy}piperidin-1-yl)-2-(2,6-dioxopiperidin-3-yl) isoindole-1,3-dione (18 mg, 4.24%) was obtained as yellow solid. Mass (m/z): 763.7 [M+H]⁺. ¹H NMR (400 MHZ, CDCl₃) δ 9.59 (s, 1H), 8.99 (s, 1H), 8.16 (s, 1H), 7.64 (dd, J=19.6, 8.4 Hz, 1H), 7.54-7.48 (m, 1H), 7.45-7.40 (m, 2H), 7.36 (d, J=7.8 Hz, 1H), 7.22 (s, 1H), 7.05-6.97 (m, 2H), 4.98-4.94 (m, 1H), 4.44 (d, J=15.2 Hz, 2H), 4.31-4.24 (m, 3H), 4.02-3.99 (m, 2H), 3.89-3.85 (m, 2H), 3.74 (s, 2H), 3.29-3.18 (m, 2H), 2.93-2.74 (m, 4H), 2.24-2.20 (m, 1H), 2.01 (dd, J=10.6, 7.8 Hz, 3H), 1.63-1.60 (m, 2H), 0.92-0.88 (m, 2H), 0.64 (d, J=5.0 Hz, 2H).

### Example 32: 3-ethyl-1H-pyrrolo 5-(4-(3-(4-(3-(4-chloro-3-ethyl-1H-pyrrolo[2,3-b]pyridin-5-yl)phenyl)-3-oxopiperazin-1-yl)-3-oxopropoxy)piperidin-1-yl)-2-(2,6-dioxopiperidin-3-yl)isoindoline-1,3-dione

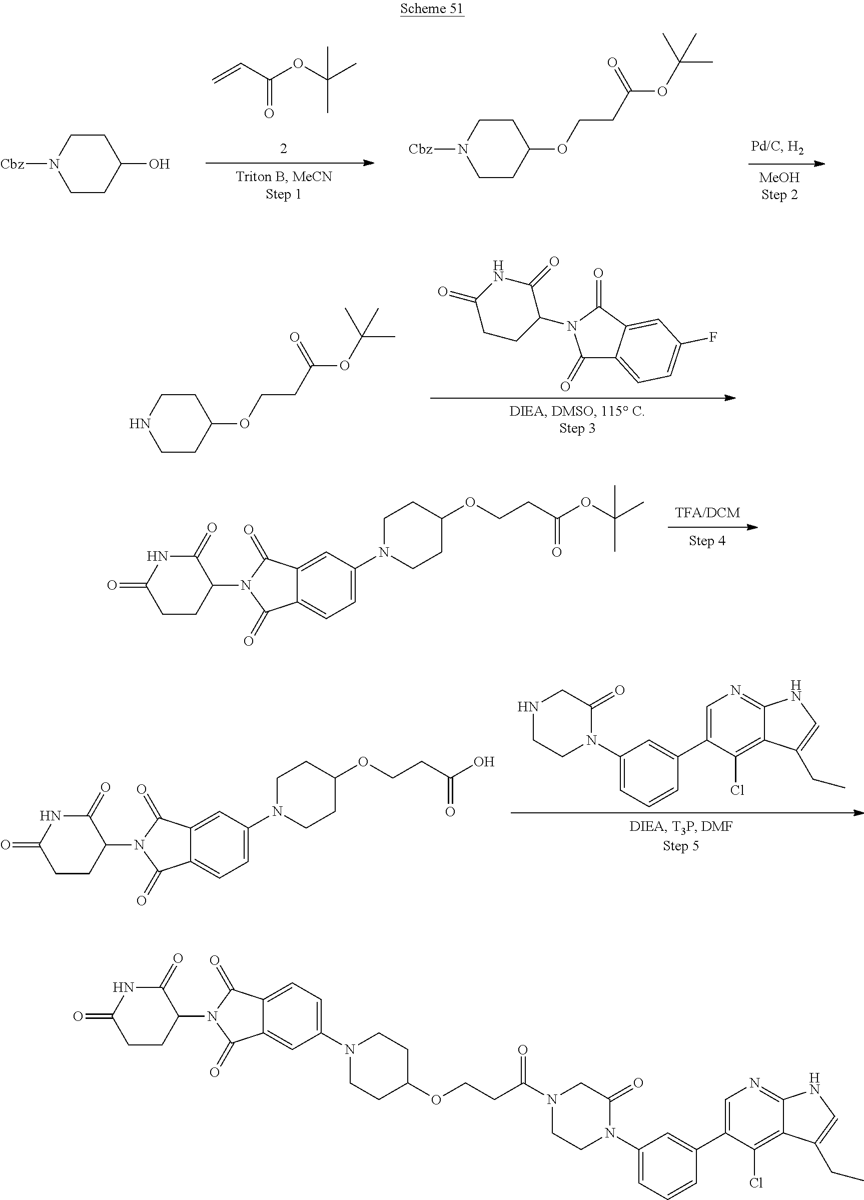

Step 1. Preparation of tert-butyl 3-[(1-{3-[(formyloxy)methyl]phenyl}piperidin-4-yl)oxy]propanoate: To a solution of [3-(4-hydroxypiperidin-1-yl)phenyl]methyl formate (2 g, 8.5 mmol) and tert-butyl prop-2-enoate (1.63 g, 0.012 mol) in MeCN (20 mL) was added Benzyltrimethylammonium hydroxide (0.13 g, 0.31 mmol). The reaction mixture was stirred at 25° C. for 16 hrs. Water (100 mL) was added and the mixture was extracted with EA (300 mL×2). The combined organic layers were washed with brine (30 mL×2), dried over Na₂SO₄. Then by filtration, the filtrate was concentrated. The crude product was purified by flash Chromatography (PE/EA=1:1) to give the product as a yellow solid (1.8 g, 49.4%). Mass (m/z): 385.9 [M+Na]⁺.

Step 2. Preparation of tert-butyl 3-(piperidin-4-yloxy) propanoate: Following General step K, the product was obtained as a light yellow with oily compound (600 mg, 52%). Mass (m/z): 230.8 [M+H]⁺.

Step 3. Preparation of tert-butyl 3-({1-[2-(2,6-dioxopiperidin-3-yl)-1,3-dioxoisoindol-5-yl]piperidin-4-yl}oxy) propanoate: Following General step L, the product was obtained as a yellow solid (450 mg, 39%). Mass (m/z): 485.8 [M+H]⁺.

Step 4. Preparation of 3-({1-[2-(2,6-dioxopiperidin-3-yl)-1,3-dioxoisoindol-5-yl]piperidin-4-yl}oxy) propanoic acid: Following General step B2, the product was obtained as a yellow solid (380 mg, 83%). Mass (m/z): 430.1 [M+H]⁺.

Step 5. Preparation of 5-(4-{3-[4-(3-{4-chloro-3-ethyl-1H-pyrrolo[2,3-b]pyridin-3-yl}phenyl)-3-oxopiperazin-1-yl]-3-oxopropoxy}piperidin-1-yl)-2-(2,6-dioxopiperidin-3-yl) isoindole-1,3-dione: Following General step F, the product was obtained as a yellow solid (36 mg, 18%). Mass (m/z): 766.1 [M+H]⁺. ¹H NMR (400 MHZ, DMSO-d₆) δ 11.79 (s, 1H), 11.08 (s, 1H), 8.11 (d, J=4.7 Hz, 1H), 7.61 (dd, J=12.7, 8.5 Hz, 1H), 7.49 (dd, J=15.0, 7.4 Hz, 2H), 7.39 (s, 3H), 7.30 (d, J=6.4 Hz, 1H), 7.24-7.13 (m, 1H), 5.06 (dd, J=12.9, 5.3 Hz, 1H), 4.33 (d, J=12.1 Hz, 1H), 4.20 (s, 1H), 3.85 (dd, J=15.2, 5.0 Hz, 4H), 3.73 (dd, J=13.2, 7.0 Hz, 4H), 3.60 (s, 1H), 3.27 (d, J=9.4 Hz, 4H), 2.95-2.86 (m, 2H), 2.72-2.55 (m, 4H), 1.88 (s, 2H), 1.49 (d, J=8.9 Hz, 2H), 1.26 (dd, J=14.2, 6.8 Hz, 3H).

### Example 33: 3-ethyl-1H-pyrrolo 5-(4-(2-(2-(4-(3-(4-chloro-3-ethyl-1H-pyrrolo[2,3-b]pyridin-5-yl)phenyl)-3-oxopiperazin-1-yl)ethoxy)ethyl)piperazin-1-yl)-2-(2,6-dioxopiperidin-3-yl)-6-fluoroisoindoline-1,3-dione

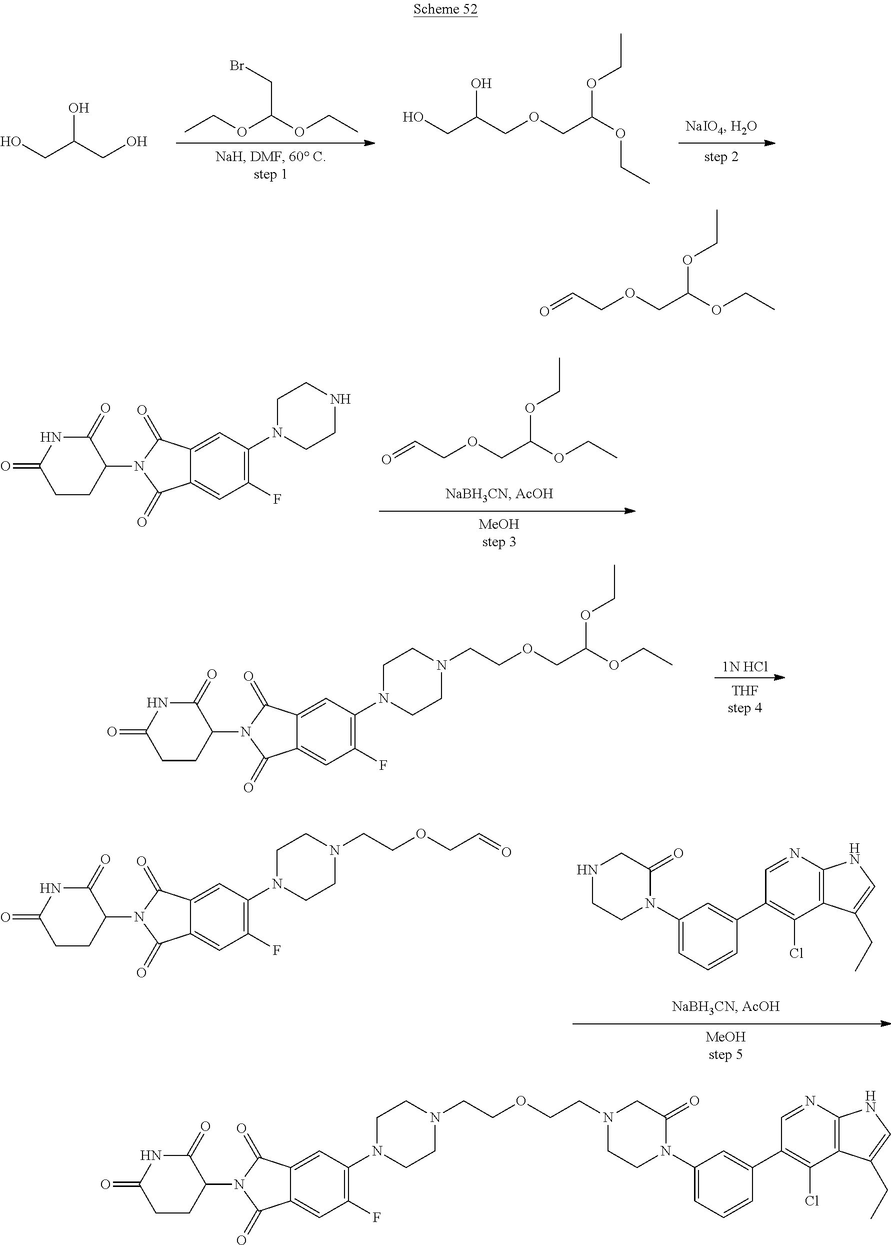

Step 1. Preparation of 3-(2,2-diethoxyethoxy) propane-1,2-diol: To a solution of propane-1,2,3-triol (1.7 g, 18 mmol) in DMF (20 mL) was added NaH (60% in oil, 1.44 g, 36 mmol) at 0° C. After 10 min, a solution of 2-bromo-1,1-diethoxyethane (1.65 g, 18 mmol) was added. The resulting mixture was stirred at 60° C. for 16 hrs. After the reaction completed, H₂O (50 mL) was added to the reaction mixture, and then extracted with EA (30 mL×5). The combined organic layer was washed with brine (50 mL×3), then dried over anhydrous Na₂SO₄. After filtration, the solution was concentrated under vacuum to give the product 3-(2,2-diethoxyethoxy) propane-1,2-diol (2 g, 54%) as colorless oil. Mass (m/z): 209 [M+H]⁺.

Step 2. Preparation of 2-(2,2-diethoxyethoxy) acetaldehyde: To a solution of 3-(2,2-diethoxyethoxy) propane-1,2-diol (1 g, 4.8 mmol) was added NaIO₄ (2 g, 9.6 mmol) in H₂O (30 mL). The resulting mixture was stirred for 1 hour at 25° C. The reaction mixture was extracted with EA (2×20 mL). The combined organic layer was washed with brine (50 mL×3), then dried over anhydrous Na₂SO₄. After filtration, the solution was concentrated under vacuum to give the product 2-(2,2-diethoxyethoxy) acetaldehyde (400 mg, 31%) as colorless oil. Mass (m/z): 177 [M+H]⁺.

Step 3. Preparation of 5-{4-[2-(2,2-diethoxyethoxy)ethyl]piperazin-1-yl}-2-(2,6-dioxopiperidin-3-yl)-6-fluoroisoindole-1,3-dione: From 2-(2,2-diethoxyethoxy) acetaldehyde (400 mg, 2.27 mmol), Following General step M1, the product was obtained (268 mg, 31%) as a yellow solid. Mass (m/z): 521 [M+H]⁺.

Step 4. Preparation of 2-(2-{4-[2-(2,6-dioxopiperidin-3-yl)-6-fluoro-1,3-dioxoisoindol-5-yl]piperazin-1-yl}ethoxy) acetaldehyde: A solution of 5-{4-[2-(2,2-diethoxyethoxy)ethyl]piperazin-1-yl}-2-(2,6-dioxopiperidin-3-yl)-6-fluoroisoindole-1,3-dione (268 mg, 0.515 mmol) in 1N HCl/THF (5 mL) was stirred for 1 hour at 25° C. After the reaction completed, H₂O (10 mL) was added to the reaction mixture, and then extracted with EA (10 mL×3). The combined organic layer was washed with brine (20 mL×2), then dried over anhydrous Na₂SO₄. After filtration, the solution was concentrated under vacuum to give the product (50 mg, 20%) as a colorless oil. Mass (m/z): 447 [M+H]⁺.

Step 5. Preparation of 5-[4-(2-{2-[4-(3-{4-chloro-3-ethyl-1H-pyrrolo[2,3-b]pyridin-3-yl}phenyl)-3-oxopiperazin-1-yl]ethoxy}ethyl)piperazin-1-yl]-2-(2,6-dioxopiperidin-3-yl)-6-fluoroisoindole-1,3-dione: Following General step M1, the product (13 mg 28%) was obtained as a yellow solid. Mass (m/z): 785 [M+H]⁺. ¹HNMR (400 MHZ, CD₃OD) δ 11.78 (s, 1H), 11.11 (s, 1H), 7.91 (t, J=8.4 Hz, 1H), 7.57 (t, J=9.2 Hz 1H), 7.45-7.35 (m, 5H), 5.12-5.08 (m, 1H), 3.74-3.71 (m, 2H), 3.60-3.58 (m, 3H), 3.33-3.25 (m, 7H), 2.93-2.84 (m, 4H), 2.56-2.47 (m, 12H), 2.07-2.02 (m, 1H), 1.28-1.23 (m, 3H).

### Example 34: 5-(4-(2-(4-(3-(4-chloro-3-ethyl-1H-pyrrolo[2,3-b]pyridin-5-yl)phenyl)-3-oxopiperazin-1-yl) acetyl)piperazin-1-yl)-2-(2,6-dioxopiperidin-3-yl)isoindoline-1,3-dione

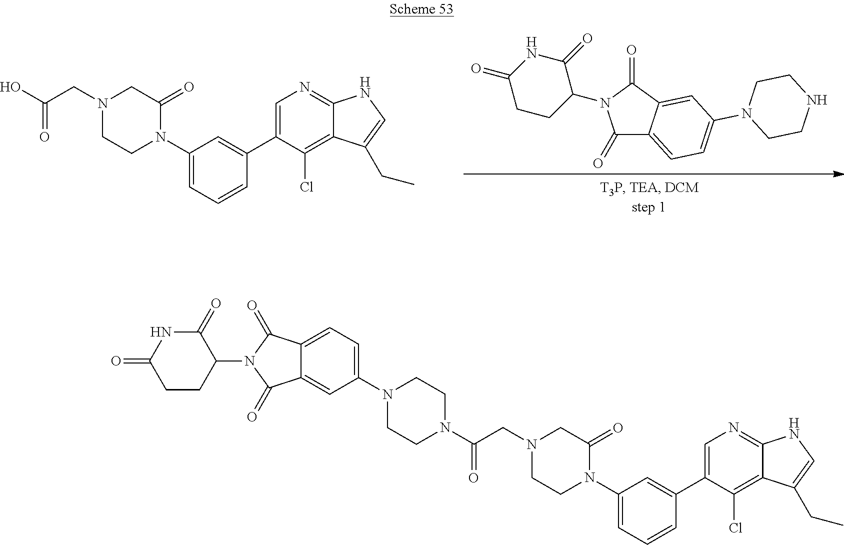 [SMILES: CCc1c[nH]c2ncc(-c3cccc(N4CCN(CC(=O)O)CC4=O)c3)c(Cl)c12 ; O=C1CCC(N2C(=O)c3ccc(N4CCNCC4)cc3C2=O)C(=O)N1 ; CCc1c[nH]c2ncc(-c3cccc(N4CCN(CC(=O)N5CCN(c6ccc7c(c6)C(=O)N(C6CCC(=O)NC6=O)C7=O)CC5)CC4=O)c3)c(Cl)c12]

Step 1. Preparation of 5-(4-(2-(4-(3-(4-chloro-3-ethyl-1H-pyrrolo[2,3-b]pyridin-5-yl)phenyl)-3-oxopiperazin-1-yl) acetyl)piperazin-1-yl)-2-(2,6-dioxopiperidin-3-yl)isoindoline-1,3-dione: Following General step F, the product (6 mg 10%) was obtained as a yellow solid. Mass (m/z): 737 [M+H]⁺. ¹HNMR (400 MHZ, CD₃OD) ô 11.79 (s, 1H), 11.08 (s, 1H), 8.12 (s, 1H), 7.71-7.50 (m, 1H), 7.48-7.25 (m, 7H), 5.10-5.05 (m, 1H), 3.75-3.33 (m, 13H), 2.91-2.84 (m, 5H), 2.67-2.07 (m, 2H), 2.03-2.00 (m, 1H), 1.29-1.23 (m, 4H).

### Example 35: 5-(4-(3-(4-(3-(4-chloro-3-ethyl-1H-pyrrolo[2,3-b]pyridin-5-yl)phenyl)-3-oxopiperazin-1-yl) propoxy)piperidin-1-yl)-2-(2,6-dioxopiperidin-3-yl)isoindoline-1,3-dione

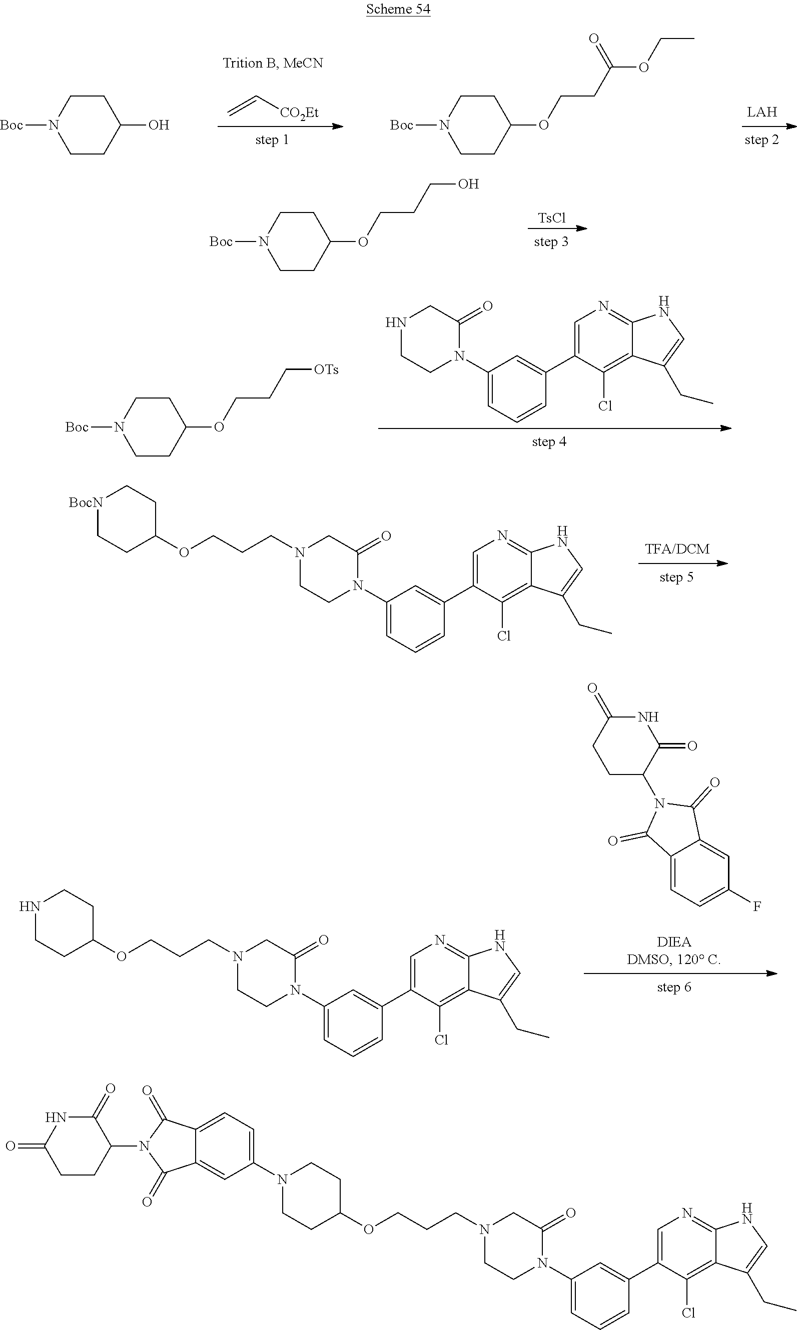

Step 1. Preparation of tert-butyl 4-(3-ethoxy-3-oxopropoxy)piperidine-1-carboxylate: To a solution of tert-butyl (4-hydroxypiperidin-1-yl) formate (3000 mg, 14.8316 mmol) in acetonitrile (30 mL) was added ethyl prop-2-enoate (2970 mg, 29.66 mmol) and Benzyltrimethylammonium hydroxide (248 mg, 0.59 mmol). The reaction mixture was stirred at 25° C. for 16 hrs. The reaction mixture was concentrated and purified by silica gel column chromatography (PE/EA=2:1) to give the product as a colorless oil (3000 mg, 46%). Mass (m/z): 246.0 [M−55]⁺.

Step 2. Preparation of tert-butyl 4-(3-hydroxypropoxy)piperidine-1-carboxylate: To a solution of ethyl 3-{[1-(tert-butyl-$1\hat{ }{3}-oxy)piperidin-4-yl]oxy}propanoate (3000 mg, 9.9211 mmol) in THF (20 mL) was added LAH (414 mg, 10.9132 mmol) at 0° C. The reaction mixture was stirred at 0° C. for 1 h. The reaction mixture was quenched by 15% wt NaOH (aqueous) (0.5 mL) and water (0.5 mL). The resulting mixture was filtered, the filtrate was concentrated to give the product as a colorless oil (2500 mg, 67%). Mass (m/z): 204.0 [M−55]⁺.

Step 3. Preparation of tert-butyl 4-(3-(tosyloxy) propoxy)piperidine-1-carboxylate: To a solution of tert-butyl [4-(3-hydroxypropoxy)piperidin-1-yl]formate (2200 mg, 8.45 mmol) in DCM (20 mL) was added TsCl (1933 mg, 10.14 mmol) and TEA (1710 mg, 16.90 mmol) at 0° C. The reaction mixture was stirred at 25° C. for 16 hrs. The reaction mixture was washed with water (50 mL) and extracted with DCM (20 mL). The organic phase was collected and evaporated to dryness. The residual was purified by silica gel column chromatography (PE/EA=3:1) to give the product as a colorless oil (2200 mg, 56%). Mass (m/z): 358.1 [M−55]⁺.

Step 4. Preparation of tert-butyl 4-(3-(4-(3-(4-chloro-3-ethyl-1H-pyrrolo[2,3-b]pyridin-5-yl)phenyl)-3-oxopiperazin-1-yl) propoxy)piperidine-1-carboxylate: Following General step G2, the product was obtained as a colorless oil (120 mg, 32%). Mass (m/z): 595.8 [M+H]⁺.

Step 5. Preparation of 1-(3-(4-chloro-3-ethyl-1H-pyrrolo[2,3-b]pyridin-5-yl)phenyl)-4-(3-(piperidin-4-yloxy) propyl)piperazin-2-one: Following General step B2, the product was obtained as a colorless oil (80 mg, 86%). Mass (m/z): 495.9 [M+H]⁺.

Step 6. Preparation of 5-(4-(3-(4-(3-(4-chloro-3-ethyl-1H-pyrrolo[2,3-b]pyridin-5-yl)phenyl)-3-oxopiperazin-1-yl) propoxy)piperidin-1-yl)-2-(2,6-dioxopiperidin-3-yl)isoindoline-1,3-dione: Following General step L, the product was obtained as a white solid (33 mg, 26%). Mass (m/z): 751.7 [M+H]⁺. ¹H NMR (400 MHZ, DMSO-d₆) δ 11.76 (s, 1H), 11.05 (s, 1H), 8.08 (s, 1H), 7.61 (d, J=8.5 Hz, 1H), 7.45 (dd, J=15.9, 8.2 Hz, 2H), 7.33 (dd, J=20.8, 11.1 Hz, 4H), 7.21 (d, J=8.7 Hz, 1H), 5.03 (dd, J=12.9, 5.3 Hz, 1H), 3.79-3.66 (m, 4H), 3.58-3.43 (m, 4H), 3.23-3.13 (m, 4H), 2.92-2.82 (m, 3H), 2.78-2.72 (m, 2H), 2.59-2.49 (m, 2H), 2.12-1.75 (m, 4H), 1.73-1.63 (m, 2H), 1.52-1.42 (m, 2H), 1.24 (t, J=7.4 Hz, 3H).

### Example 36: 3-(2,2-difluoroethyl)-1H-pyrrolo 5-(4-(2-(4-(3-(4-chloro-3-(2,2-difluoroethyl)-1H-pyrrolo[2,3-b]pyridin-5-yl)phenyl)-3-oxopiperazin-1-yl)-2-oxoethoxy)piperidin-1-yl)-2-(2,6-dioxopiperidin-3-yl)isoindoline-1,3-dione

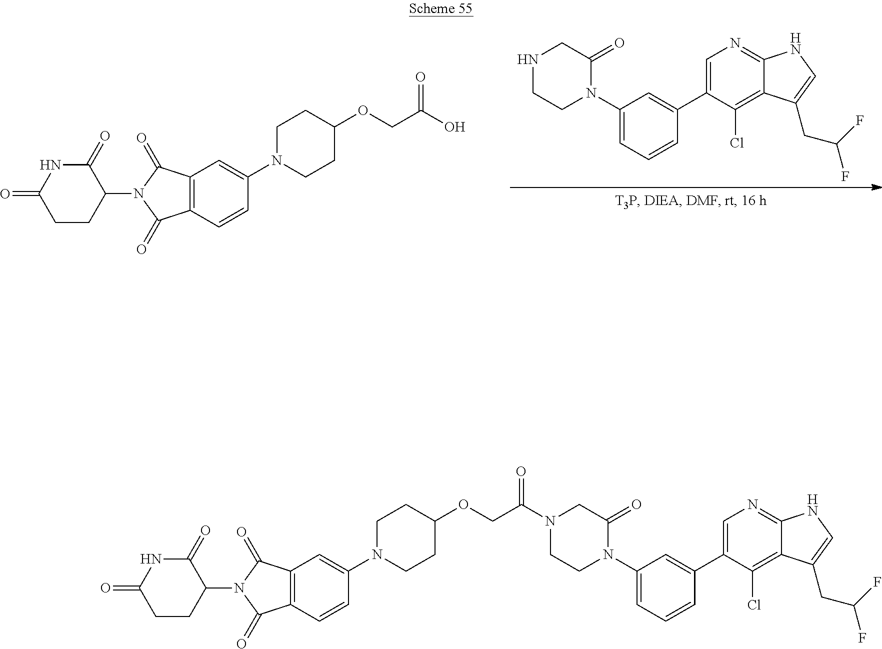 [SMILES: O=C(O)COC1CCN(c2ccc3c(c2)C(=O)N(C2CCC(=O)NC2=O)C3=O)CC1 ; O=C1CNCCN1c1cccc(-c2cnc3[nH]cc(CC(F)F)c3c2Cl)c1 ; O=C1CCC(N2C(=O)c3ccc(N4CCC(OCC(=O)N5CCN(c6cccc(-c7cnc8[nH]cc(CC(F)F)c8c7Cl)c6)C(=O)C5)CC4)cc3C2=O)C(=O)N1]

Step 1. Preparation of 5-{4-[2-(4-{3-[4-chloro-3-(2,2-difluoroethyl)-1H-pyrrolo[2,3-b]pyridin-3-yl]phenyl}-3-oxopiperazin-1-yl)-2-oxoethoxy]piperidin-1-yl}-2-(2,6-dioxopiperidin-3-yl) isoindole-1,3-dione: Following General step F, the product was obtained as a yellow solid (36 mg, 18%). Mass (m/z): 787.6 [M+H]⁺. ¹H NMR (400 MHZ, CDCl₃) δ 12.57 (s, 1H), 8.35 (s, 1H), 8.18 (s, 1H), 7.66 (t, J=8.2 Hz, 1H), 7.57 (t, J=7.9 Hz, 1H), 7.47 (s, 1H), 7.42-7.36 (m, 3H), 7.06 (t, J=7.4 Hz, 1H), 6.08 (tt, J=56.4, 4.3 Hz, 1H), 4.94 (dd, J=12.2, 5.2 Hz, 1H), 4.44 (d, J=5.0 Hz, 2H), 4.29 (s, 2H), 4.01 (d, J=5.2 Hz, 2H), 3.86 (d, J=5.8 Hz, 2H), 3.73 (s, 3H), 3.59 (td, J=16.6, 4.2 Hz, 2H), 3.30-3.25 (m, 2H), 2.91-2.70 (m, 3H), 2.15-2.11 (m, 1H), 2.03-2.01 (m, 2H), 1.79-1.66 (m, 2H).

### Example 37: 3-ethyl-1H-pyrrolo N-(2-(4-(3-(4-chloro-3-ethyl-1H-pyrrolo[2,3-b]pyridin-5-yl)phenyl)-3-oxopiperazin-1-yl)ethyl)-1-(2-(2,6-dioxopiperidin-3-yl)-1,3-dioxoisoindolin-5-yl)piperidine-4-carboxamide

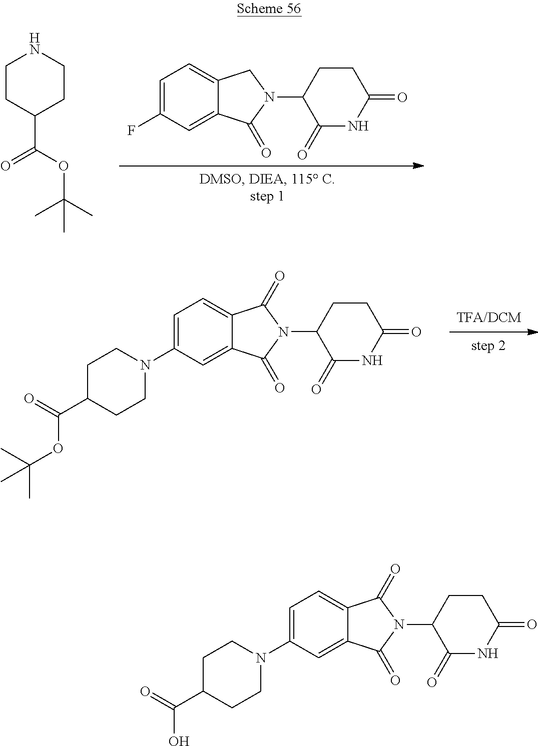 [SMILES: CC(C)(C)OC(=O)C1CCNCC1 ; O=C1CCC(N2Cc3ccc(F)cc3C2=O)C(=O)N1 ; CC(C)(C)OC(=O)C1CCN(c2ccc3c(c2)C(=O)N(C2CCC(=O)NC2=O)C3=O)CC1 ; O=C1CCC(N2C(=O)c3ccc(N4CCC(C(=O)O)CC4)cc3C2=O)C(=O)N1]

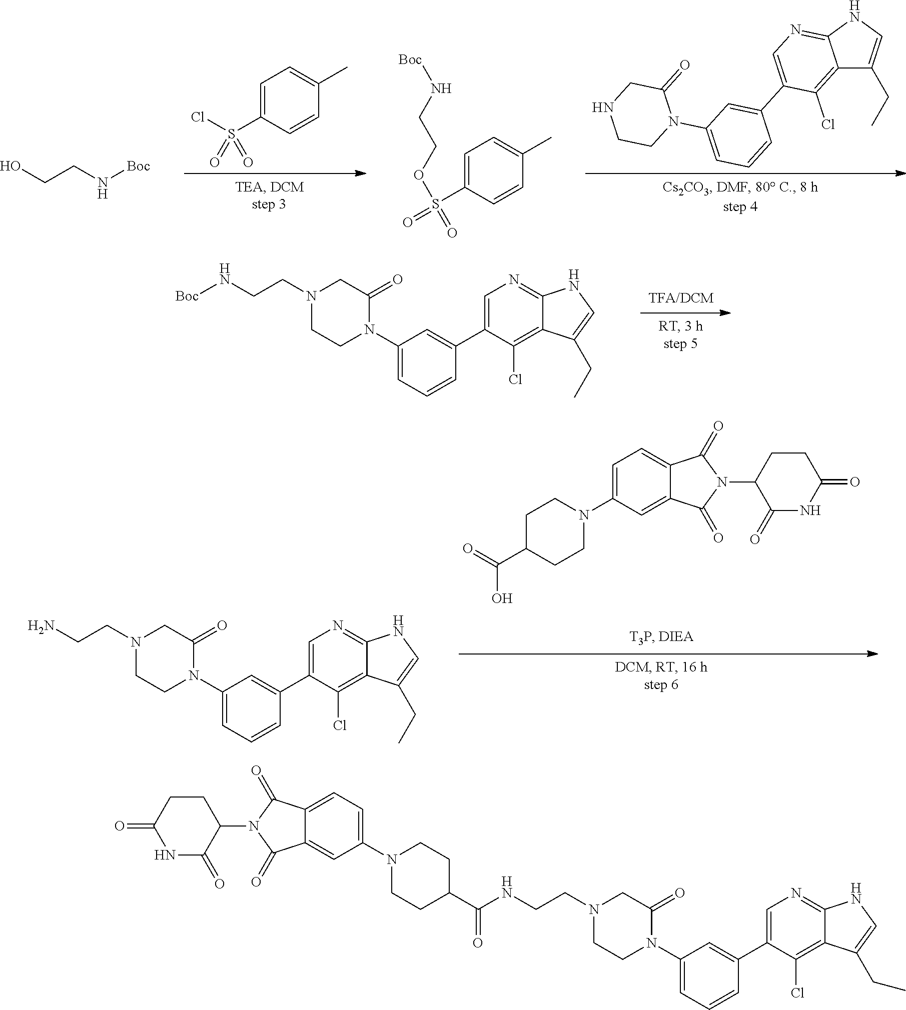

Step 1. Preparation of tert-butyl 1-[2-(2,6-dioxopiperidin-3-yl)-1,3-dioxoisoindol-5-yl]piperidine-4-carboxylate: Following General step L, the product tert-butyl 1-[2-(2,6-dioxopiperidin-3-yl)-1,3-dioxoisoindol-5-yl]piperidine-4-carboxylate (580 mg, 73%) was obtained as a yellow solid. Mass (m/z): 441.9 [M+H]⁺.

Step 2. Preparation of 1-[2-(2,6-dioxopiperidin-3-yl)-1,3-dioxoisoindol-5-yl]piperidine-4-carboxylic acid: Following General step B2, the product 1-[2-(2,6-dioxopiperidin-3-yl)-1,3-dioxoisoindol-5-yl]piperidine-4-carboxylic acid (400 mg, 86%) was obtained as a brown solid. Mass (m/z): 386.1 [M+H]⁺.

Step 3. Preparation of tert-butyl (2-{[(4-methylbenzene) sulfonyl]oxy}ethyl)amino formate: To a mixture of tert-butyl (2-hydroxyethyl)amino formate (1.0 g, 6.20 mmol) in DCM (15 mL) was added TEA (1.88 g, 18.6 mmol) and 4-methylbenzenesulfonyl chloride (1.77 g, 9.3 mmol) at 0° C. The reaction was stirred at rt under N₂ for 2 hrs. The reaction mixture was concentrated under reduced pressure. The residue was purified by flash column (PE/EA=0˜ 13%) to give the product tert-butyl (2-{[(4-methylbenzene) sulfonyl]oxy}ethyl)amino formate (1.1 g, 56%) as colorless oil. Mass (m/z): 337.9 [M+Na]⁺.

Step 4. Preparation of tert-butyl {2-[4-(3-{4-chloro-3-ethyl-1H-pyrrolo[2,3-b]pyridin-3-yl}phenyl)-3-oxopiperazin-1-yl]ethyl}amino formate: To a mixture of tert-butyl (2-{[(4-methylbenzene) sulfonyl]oxy}ethyl)amino formate (1.0 g, 3.1 mmol) in DMF (15 mL) was added Cs₂CO₃ (3.0 g, 9.30 mmol) and 1-(3-{4-chloro-3-ethyl-1H-pyrrolo[2,3-b]pyridin-3-yl}phenyl)piperazin-2-one (0.75 g, 2.1 mmol). The reaction was stirred at 80° C. under N₂ for 16 hrs. After the reaction completed, H₂O (30 mL) was added to the reaction mixture, and then extracted with EA (30 mL×3). The combined organic layer was washed with brine (30 mL×3), then dried over anhydrous Na₂SO₄. After filtration, the filtrate was concentrated under vacuum, and the residue was purified by flash column (PE/EA=0˜50%) to give the product tert-butyl {2-[4-(3-{4-chloro-3-ethyl-1H-pyrrolo[2,3-b]pyridin-3-yl}phenyl)-3-oxopiperazin-1-yl]ethyl}amino formate (700 mg, 39%) as a yellow solid. Mass (m/z): 487.9 [M+H]⁺.

Step 5. Preparation of 4-(2-aminoethyl)-1-(3-{4-chloro-3-ethyl-1H-pyrrolo[2,3-b]pyridin-3-yl}phenyl)piperazin-2-one: Following General step B2, the product 4-(2-aminoethyl)-1-(3-{4-chloro-3-ethyl-1H-pyrrolo[2,3-b]pyridin-3-yl}phenyl)piperazin-2-one (550 mg, 89%) was obtained as a brown solid. Mass (m/z): 398.0 [M+H]⁺.

Step 6. Preparation of N-{2-[4-(3-{4-chloro-3-ethyl-1H-pyrrolo[2,3-b]pyridin-3-yl}phenyl)-3-oxopiperazin-1-yl]ethyl}-1-[2-(2,6-dioxopiperidin-3-yl)-1,3-dioxoisoindol-5-yl]piperidine-4-carboxamide: Following General step F, the product (5.0 mg, 1%) was obtained as a yellow solid. Mass (m/z): 764.7 [M+H]⁺. ¹H NMR (400 MHZ, CD₃OD) δ 8.14 (s, 1H), 7.98 (t, J=5.8 Hz, 1H), 7.64-7.56 (m, 2H), 7.49-7.41 (m, 3H), 7.31 (d, J=2.0 Hz, 1H), 7.28 (s, 1H), 7.19-7.16 (m, 1H), 4.42-4.40 (m, 2H), 4.02 (s, 3H), 3.96 (d, J=13.0 Hz, 2H), 3.69 (s, 2H), 3.64 (dd, J=11.2, 5.8 Hz, 1H), 3.01-2.68 (m, 7H), 2.34 (t, J=11.2 Hz, 1H), 2.11-2.09 (m, 1H), 1.70 (d, J=12.2 Hz, 2H), 1.64-1.57 (m, 2H), 1.32 (t, J=7.4 Hz, 5H).

### Example 38: 5-(4-((4-(3-(4-chloro-3-ethyl-1H-pyrrolo[2,3-b]pyridin-5-yl)phenyl)-3-oxo piperazin-1-yl)methyl)piperidin-1-yl)-2-(2,6-dioxopiperidin-3-yl)-6-fluoro isoindoline-1,3-dione

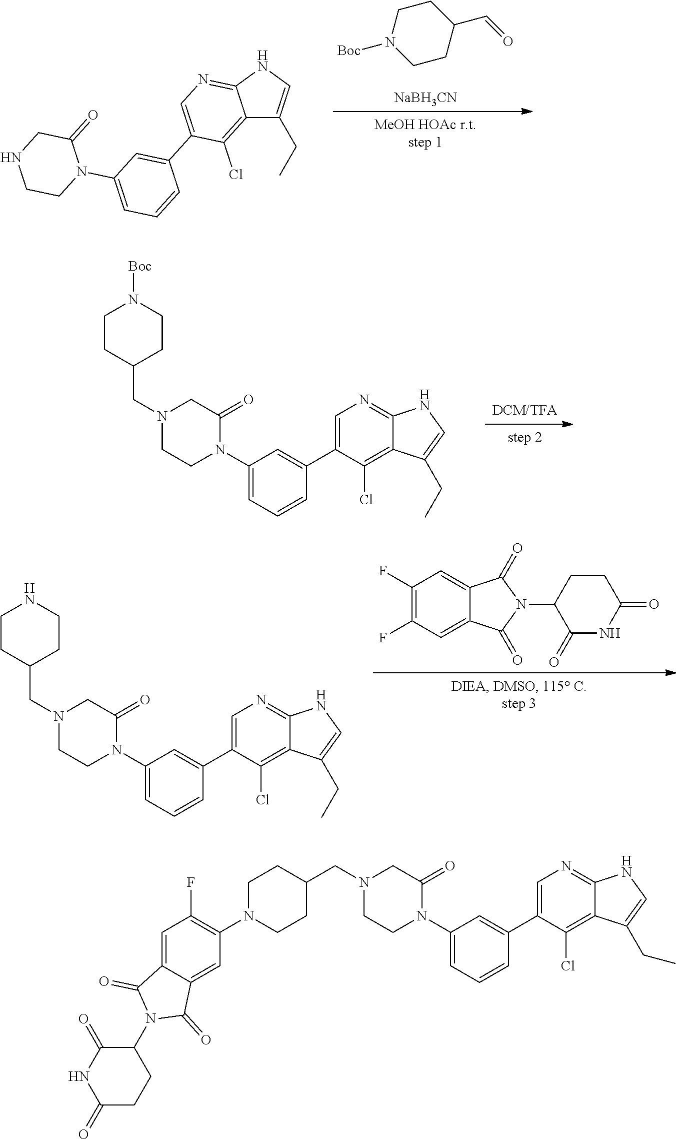

Step 1. Preparation of tert-butyl 4-((4-(3-(4-chloro-3-ethyl-1H-pyrrolo[2,3-b]pyridin-5-yl)phenyl)-3-oxopiperazin-1-yl)methyl)piperidine-1-carboxylate: From 1-(3-(4-chloro-3-ethyl-1H-pyrrolo[2,3-b]pyridin-5-yl)phenyl)piperazin-2-one (300 mg, 0.85 mmol) and tert-butyl 4-formylpiperidine-1-carboxylate (362 mg, 1.69 mmol), following General step M1, the product tert-butyl 4-((4-(3-(4-chloro-3-ethyl-1H-pyrrolo[2,3-b]pyridin-5-yl)phenyl)-3-oxopiperazin-1-yl)methyl)piperidine-1-carboxylate was obtained as yellow oil (277 mg, 59%). Mass (m/z): 522.0 [M+H]⁺.

Step 2. Preparation of 1-(3-(4-chloro-3-ethyl-1H-pyrrolo[2,3-b]pyridin-5-yl)phenyl)-4-(piperidin-4-ylmethyl)piperazin-2-one: Following General step B2, the product 1-(3-(4-chloro-3-ethyl-1H-pyrrolo[2,3-b]pyridin-5-yl)phenyl)-4-(piperidin-4-ylmethyl)piperazin-2-one was obtained as yellow oil (400 mg, purity: 60%). Mass (m/z): 451.9 [M+H]⁺.

Step 3. Preparation of 5-(4-((4-(3-(4-chloro-3-ethyl-1H-pyrrolo[2,3-b]pyridin-5-yl)phenyl)-3-oxopiperazin-1-yl)methyl)piperidin-1-yl)-2-(2,6-dioxopiperidin-3-yl)-6-fluoroisoindoline-1,3-dione: Following General step L, the product was obtained as a yellow solid (60 mg, 18%). Mass (m/z): 726.3 [M+H]⁺. ¹H NMR (400 MHZ, CD₃OD) δ 8.15 (s, 1H), 7.67-7.46 (m, 5H), 7.32 (s, 1H), 5.11 (dd, J=12.6, 5.4 Hz, 1H), 4.94 (s, 1H), 4.89-4.86 (m, 2H), 4.16 (dd, J=18.8, 13.6 Hz, 4H), 3.94-3.67 (m, 4H), 3.08-2.97 (m, 3H), 2.91-2.83 (m, 1H), 2.76 (dd, J=22.2, 7.8 Hz, 2H), 2.27-2.09 (m, 2H), 2.06-1.97 (m, 2H), 1.60 (d, J=9.0 Hz, 2H), 1.36 (t, J=7.4 Hz, 3H), 1.31 (d, J=3.4 Hz, 1H).

### Example 39: 5-(4-(3-(4-(3-(4-chloro-3-(2,2-difluoroethyl)-1H-pyrrolo[2,3-b]pyridin-5-yl)phenyl)-3-oxopiperazin-1-yl) propoxy)piperidin-1-yl)-2-(2,6-dioxopiperidin-3-yl)-6-fluoroisoindoline-1,3-dione

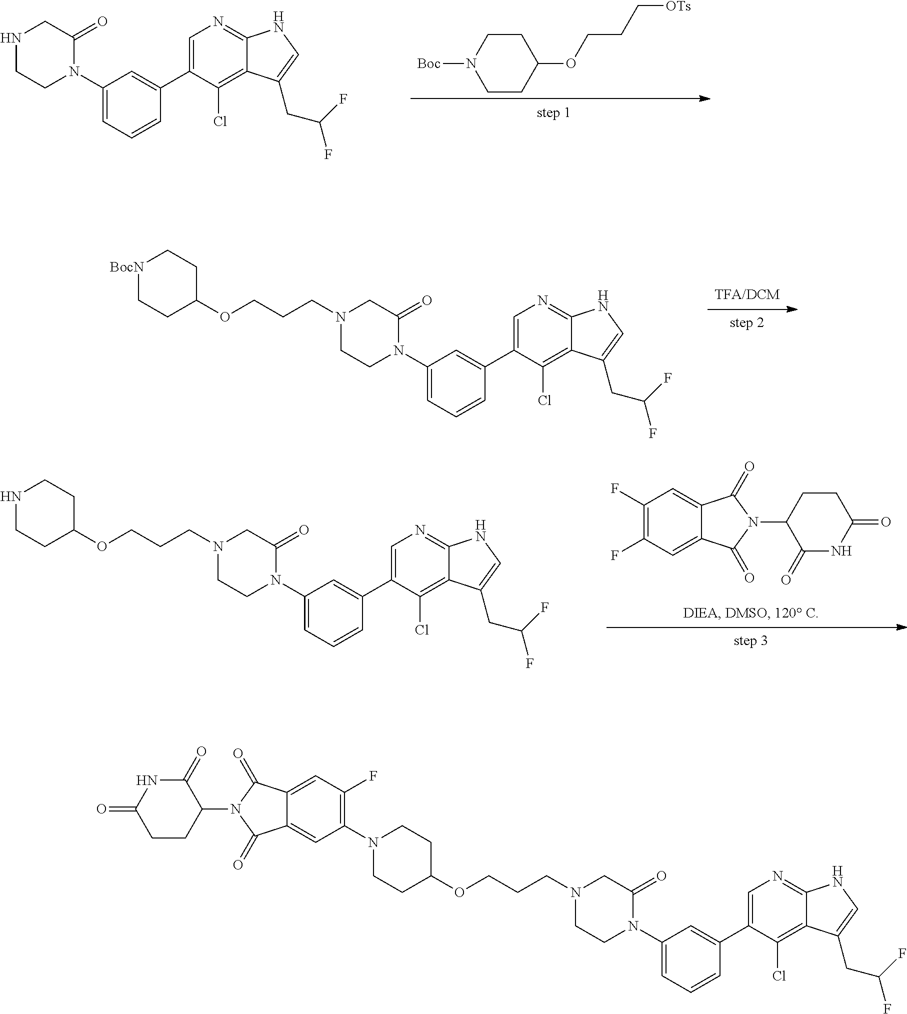 [SMILES: O=C1CNCCN1c1cccc(-c2cnc3[nH]cc(CC(F)F)c3c2Cl)c1 ; Cc1ccc(S(=O)(=O)OCCCOC2CCN(C(=O)OC(C)(C)C)CC2)cc1 ; CC(C)(C)OC(=O)N1CCC(OCCCN2CCN(c3cccc(-c4cnc5[nH]cc(CC(F)F)c5c4Cl)c3)C(=O)C2)CC1 ; O=C1CN(CCCOC2CCNCC2)CCN1c1cccc(-c2cnc3[nH]cc(CC(F)F)c3c2Cl)c1 ; O=C1CCC(N2C(=O)c3cc(F)c(F)cc3C2=O)C(=O)N1 ; O=C1CCC(N2C(=O)c3cc(F)c(N4CCC(OCCCN5CCN(c6cccc(-c7cnc8[nH]cc(CC(F)F)c8c7Cl)c6)C(=O)C5)CC4)cc3C2=O)C(=O)N1]

Step 1. Preparation of tert-butyl 4-(3-(4-(3-(4-chloro-3-(2,2-difluoroethyl)-1H-pyrrolo[2,3-b]pyridin-5-yl)phenyl)-3-oxopiperazin-1-yl) propoxy)piperidine-1-carboxylate: Following General step G2, the product was obtained as a yellow oil (100 mg, 39%). Mass (m/z): 632.2 [M+H]⁺.

Step 2. Preparation of 1-(3-(4-chloro-3-(2,2-difluoroethyl)-1H-pyrrolo[2,3-b]pyridin-5-yl)phenyl)-4-(3-(piperidin-4-yloxy) propyl)piperazin-2-one: Following General step B2, the product was obtained as a yellow oil (140 mg, 99%). Mass (m/z): 532.2 [M+H]⁺.

Step 3. Preparation of 5-(4-(3-(4-(3-(4-chloro-3-(2,2-difluoroethyl)-1H-pyrrolo[2,3-b]pyridin-5-yl)phenyl)-3-oxopiperazin-1-yl) propoxy)piperidin-1-yl)-2-(2,6-dioxopiperidin-3-yl)-6-fluoroisoindoline-1,3-dione: Following General step L, the product was obtained as a yellow solid (41 mg, 20%). Mass (m/z): 805.6 [M+H]⁺. ¹H NMR (400 MHZ, DMSO-d₆) δ 12.16 (s, 1H), 11.12 (s, 1H), 8.19 (s, 1H), 7.72 (d, J=11.4 Hz, 1H), 7.64-7.39 (m, 6H), 6.29 (tt, J=56.7, 4.5 Hz, 1H), 5.11 (dd, J=12.8, 5.4 Hz, 1H), 4.14-4.11 (m, 2H), 4.02-3.98 (m, 2H), 3.82-3.65 (m, 2H), 3.60-3.45 (m, 7H), 3.38-3.28 (m, 2H), 3.07 (t, J=9.9 Hz, 2H), 2.95-2.82 (m, 1H), 2.64-2.51 (m, 2H), 2.08-1.93 (m, 5H), 1.69-1.56 (m, 2H).

### Example 40: 5-(4-(3-(4-(3-(4-chloro-3-cyclopropyl-1H-pyrrolo[2,3-b]pyridin-5-yl)phenyl)-3-oxopiperazin-1-yl) propoxy)piperidin-1-yl)-2-(2,6-dioxopiperidin-3-yl)-6-fluoroisoindoline-1,3-dione

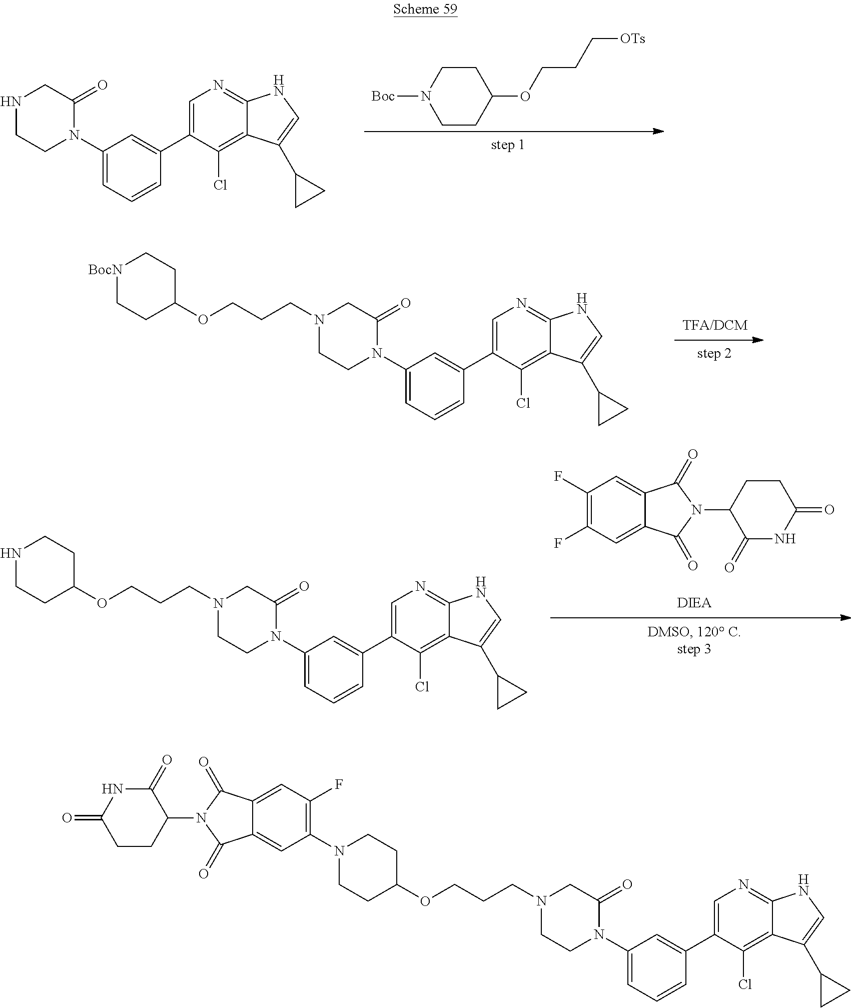 [SMILES: O=C1CNCCN1c1cccc(-c2cnc3[nH]cc(C4CC4)c3c2Cl)c1 ; Cc1ccc(S(=O)(=O)OCCCOC2CCN(C(=O)OC(C)(C)C)CC2)cc1 ; CC(C)(C)OC(=O)N1CCC(OCCCN2CCN(c3cccc(-c4cnc5[nH]cc(C6CC6)c5c4Cl)c3)C(=O)C2)CC1 ; O=C1CN(CCCOC2CCNCC2)CCN1c1cccc(-c2cnc3[nH]cc(C4CC4)c3c2Cl)c1 ; O=C1CCC(N2C(=O)c3cc(F)c(F)cc3C2=O)C(=O)N1 ; O=C1CCC(N2C(=O)c3cc(F)c(N4CCC(OCCCN5CCN(c6cccc(-c7cnc8[nH]cc(C9CC9)c8c7Cl)c6)C(=O)C5)CC4)cc3C2=O)C(=O)N1]

Step 1. Preparation of tert-butyl 4-(3-(4-(3-(4-chloro-3-cyclopropyl-1H-pyrrolo[2,3-b]pyridin-5-yl)phenyl)-3-oxopiperazin-1-yl) propoxy)piperidine-1-carboxylate: Following General step G2, the product was obtained as a yellow oil (850 mg, 54%). Mass (m/z): 607.8 [M+H]⁺.

Step 2. Preparation of 1-(3-(4-chloro-3-cyclopropyl-1H-pyrrolo[2,3-b]pyridin-5-yl)phenyl)-4-(3-(piperidin-4-yloxy) propyl)piperazin-2-one: Following General step B2, the product was obtained as a yellow oil (1000 mg, 98%). Mass (m/z): 508.2 [M+H]⁺.

Step 3. Preparation of 5-(4-(3-(4-(3-(4-chloro-3-cyclopropyl-1H-pyrrolo[2,3-b]pyridin-5-yl)phenyl)-3-oxopiperazin-1-yl) propoxy)piperidin-1-yl)-2-(2,6-dioxopiperidin-3-yl)-6-fluoroisoindoline-1,3-dione: Following General step L, the product was obtained as a yellow solid (620 mg, 40%). Mass (m/z): 781.7 [M+H]⁺. ¹H NMR (400 MHZ, DMSO-d₆) δ 11.76 (d, J=2.4 Hz, 1H), 11.12 (s, 1H), 8.12 (s, 1H), 7.69 (d, J=11.5 Hz, 1H), 7.53-7.37 (m, 5H), 7.32-7.28 (m, 1H), 5.10 (dd, J=12.8, 5.4 Hz, 1H), 3.83-3.69 (m, 2H), 3.57-3.36 (m, 9H), 3.09-3.02 (m, 2H), 2.93-2.82 (m, 2H), 2.64-2.51 (m, 3H), 2.23-2.16 (m, 1H), 2.05-1.93 (m, 3H), 1.81-1.70 (m, 2H), 1.65-1.55 (m, 2H), 0.87-0.81 (m, 2H), 0.66-0.61 (m, 2H).

### Example 41: 3-(2,2-difluoroethyl)-1H-pyrrolo 5-(4-(3-(4-(3-(4-chloro-3-(2,2-difluoroethyl)-1H-pyrrolo[2,3-b]pyridin-5-yl)phenyl)-3-oxopiperazin-1-yl) propyl)piperazin-1-yl)-2-(2,6-dioxopiperidin-3-yl)-6-fluoroisoindoline-1,3-dione

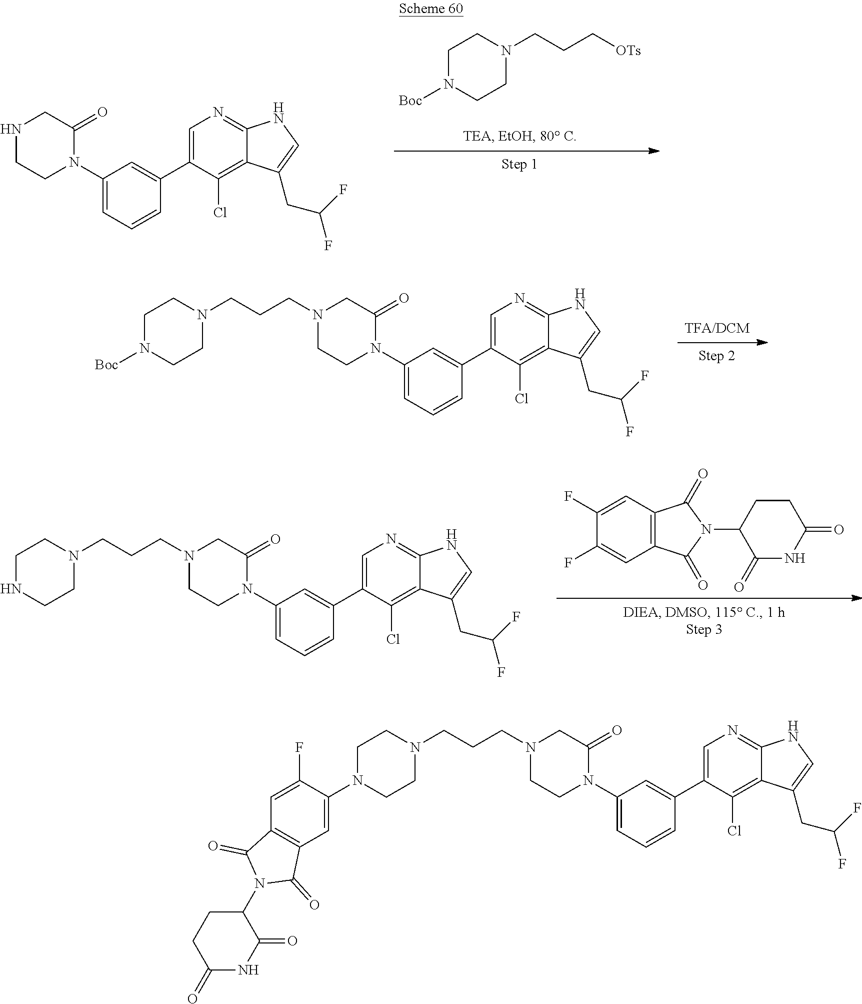 [SMILES: Cc1ccc(S(=O)(=O)OCCCN2CCN(C(=O)OC(C)(C)C)CC2)cc1 ; O=C1CNCCN1c1cccc(-c2cnc3[nH]cc(CC(F)F)c3c2Cl)c1 ; CC(C)(C)OC(=O)N1CCN(CCCN2CCN(c3cccc(-c4cnc5[nH]cc(CC(F)F)c5c4Cl)c3)C(=O)C2)CC1 ; O=C1CN(CCCN2CCNCC2)CCN1c1cccc(-c2cnc3[nH]cc(CC(F)F)c3c2Cl)c1 ; O=C1CCC(N2C(=O)c3cc(F)c(F)cc3C2=O)C(=O)N1 ; O=C1CCC(N2C(=O)c3cc(F)c(N4CCN(CCCN5CCN(c6cccc(-c7cnc8[nH]cc(CC(F)F)c8c7Cl)c6)C(=O)C5)CC4)cc3C2=O)C(=O)N1]

Step 1. Preparation of tert-butyl {4-[3-(4-{3-[4-chloro-3-(2,2-difluoroethyl)-1H-pyrrolo[2,3-b]pyridin-3-yl]phenyl}-3-oxopiperazin-1-yl) propyl]piperazin-1-yl}formate: Following General step G2, the product (120 mg, 79.24%) was obtained as yellow oil. Mass (m/z): 617.1 [M+H]⁺.

Step 2. Preparation of 1-{3-[4-chloro-3-(2,2-difluoroethyl)-1H-pyrrolo[2,3-b]pyridin-3-yl]phenyl}-4-[3-(piperazin-1-yl) propyl]piperazin-2-one: Following General step B2, the desired product (100 mg, 88.67%) was obtained as a yellow solid. Mass (m/z): 517.1 [M+H]⁺.

Step 3. Preparation of 5-{4-[3-(4-{3-[4-chloro-3-(2,2-difluoroethyl)-1H-pyrrolo[2,3-b]pyridin-3-yl]phenyl}-3-oxopiperazin-1-yl) propyl]piperazin-1-yl}-2-(2,6-dioxopiperidin-3-yl)-6-fluoroisoindole-1,3-dione: Following General step L, the desired product (46 mg, 30%) was obtained as a yellow solid. Mass (m/z): 791.2 [M+H]⁺. ¹H NMR (400 MHZ, DMSO-d₆) δ 12.13 (d, J=2.2 Hz, 1H), 11.13 (s, 1H), 8.18 (s, 1H), 7.83 (d, J=11.1 Hz, 1H), 7.61 (dd, J=8.6, 4.9 Hz, 2H), 7.55 (m, 1H), 7.48 (s, 1H), 7.42 (dd, J=7.7, 1.4 Hz, 2H), 6.43-6.15 (m, OH), 5.13 (dd, J=12.8, 5.4 Hz, 1H), 3.88 (s, 2H), 3.55 (s, 10H), 3.24 (m, 4H), 2.86 (m, 2H), 2.62 (m, 2H), 2.38 (m, 2H), 2.04 (m, 4H).

### Example 42: 3-cyclopropyl-1H-pyrrolo 5-(4-((2-(4-(3-(4-chloro-3-cyclopropyl-1H-pyrrolo[2,3-b]pyridin-5-yl)phenyl)-3-oxopiperazin-1-yl)-2-oxoethyl)(methyl)amino)piperidin-1-yl)-2-(2,6-dioxopiperidin-3-yl)-6-fluoroisoindoline-1,3-dione

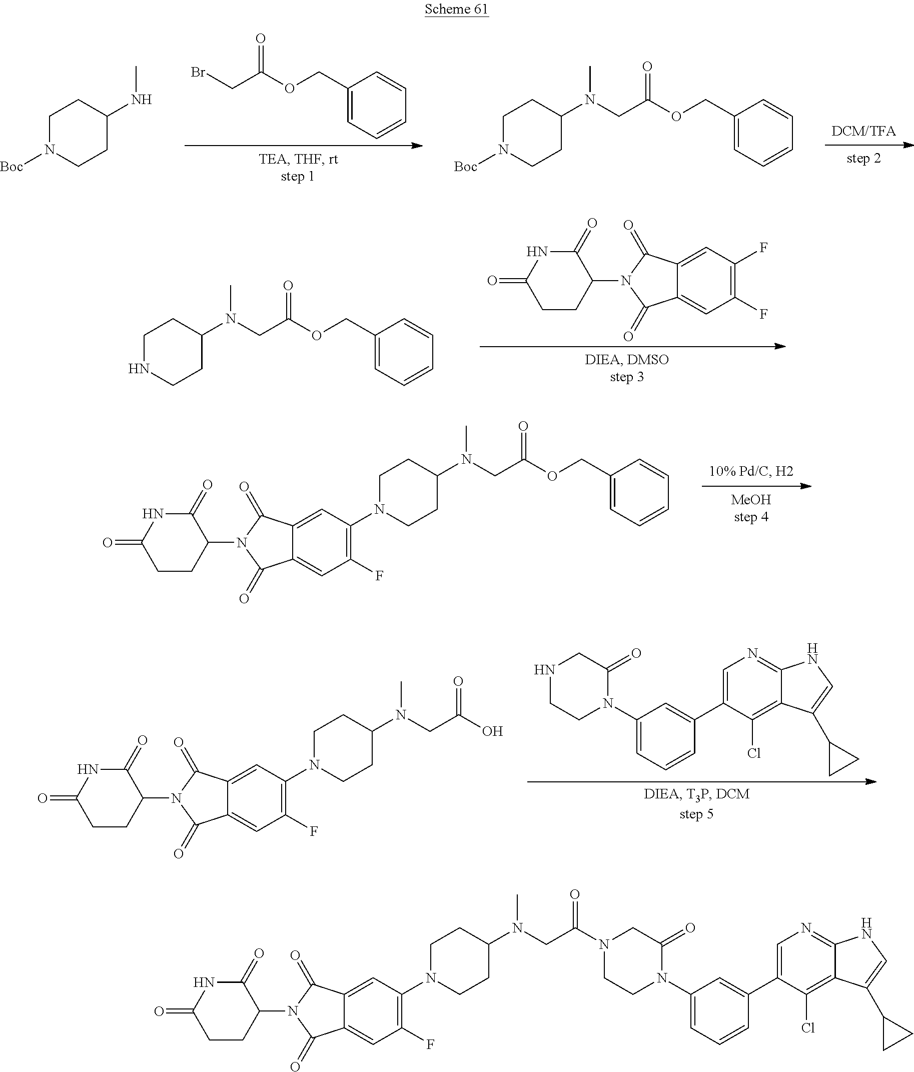

Step 1. Preparation of tert-butyl 4-((2-(benzyloxy)-2-oxoethyl)(methyl)amino) piperidine-1-carboxylate: To a mixture of tert-butyl [4-(methylamino)piperidin-1-yl]formate (5.0 g, 0.023 mol) in THF (35 mL) was added TEA (7.04 g, 0.069 mol) and benzyl 2-bromoacetate (6.38 g, 0.027 mol). The reaction was stirred at rt for 2 hrs. The reaction mixture was concentrated under reduced pressure. The residue was diluted with water (100 mL), extracted with EA (100 mL×3). The combined organic layer was washed with brine (100 mL), dried over anhydrous Na₂SO₄ and concentrated under reduced pressure. The residue was purified by flash column (PE/EA=0˜13%) to give the product tert-butyl 4-((2-(benzyloxy)-2-oxoethyl)(methyl)amino)piperidine-1-carboxylate as a white solid (3.0 g, 32%). Mass (m/z): 363.0 [M+H]⁺.

Step 2. Preparation of benzyl 2-[methyl(piperidin-4-yl)amino]acetate: From tert-butyl 4-((2-(benzyloxy)-2-oxoethyl)(methyl)amino)piperidine-1-carboxylate (1.0 g, 2.8 mmol), following General step B2, the product benzyl 2-[methyl(piperidin-4-yl)amino]acetate (1.5 g, 93%) was obtained as a brown solid. Mass (m/z): 263.0 [M+H]⁺.

Step 3. Preparation of benzyl 2-({1-[2-(2,6-dioxopiperidin-3-yl)-6-fluoro-1,3-dioxoisoindol-5-yl]piperidin-4-yl}(methyl)amino)acetate: Following General step L, the product benzyl 2-({1-[2-(2,6-dioxopiperidin-3-yl)-6-fluoro-1,3-dioxoisoindol-5-yl]piperidin-4-yl}(methyl)amino)acetate (1.0 g, 46%) was obtained as a yellow solid. Mass (m/z): 536.8 [M+H]⁺.

Step 4. Preparation of ({1-[2-(2,6-dioxopiperidin-3-yl)-6-fluoro-1,3-dioxoisoindol-5-yl]piperidin-4-yl}(methyl)amino) acetic acid: Following General step K, the product (50.0 mg, 99%) was obtained as a white solid. Mass (m/z): 477.1 [M+H]⁺.

Step 5. Preparation of 5-[4-({2-[4-(3-{4-chloro-3-cyclopropyl-1H-pyrrolo[2,3-b]pyridin-3-yl}phenyl)-3-oxopiperazin-1-yl]-2-oxoethyl}(methyl)amino)piperidin-1-yl]-2-(2,6-dioxopiperidin-3-yl)-6-fluoroisoindole-1,3-dione: Following General step F, the product (12 mg, 13%) was obtained as a yellow solid. Mass (m/z): 794.7 [M+H]⁺. ¹H NMR (400 MHZ, CD₃OD) δ 8.12 (s, 1H), 7.58 (dd, J=9.2, 3.8 Hz, 2H), 7.51 (dd, J=16.2, 7.6 Hz, 3H), 7.43 (d, J=7.8 Hz, 1H), 7.18 (d, J=0.8 Hz, 1H), 5.10 (dd, J=12.4, 5.4 Hz, 1H), 4.55 (d, J=16.8 Hz, 1H), 4.43 (s, 1H), 4.34 (s, 1H), 4.29 (d, J=16.0 Hz, 1H), 3.99 (d, J=5.5 Hz, 1H), 3.92 (d, J=4.6 Hz, 2H), 3.85 (d, J=12.0 Hz, 2H), 3.58 (s, 1H), 3.00 (dd, J=14.8, 7.6 Hz, 5H), 2.90-2.82 (m, 1H), 2.77-2.66 (m, 2H), 2.27-2.03 (m, 7H), 0.93-0.89 (m, 2H), 0.67-0.63 (m, 2H).

### Example 43: 3-cyclopropyl-1H-pyrrolo 5-(4-(3-(4-(3-(4-chloro-3-cyclopropyl-1H-pyrrolo[2,3-b]pyridin-5-yl)phenyl)-3-oxopiperazin-1-yl) propyl)piperazin-1-yl)-2-(2,6-dioxopiperidin-3-yl)-6-fluoroisoindoline-1,3-dione

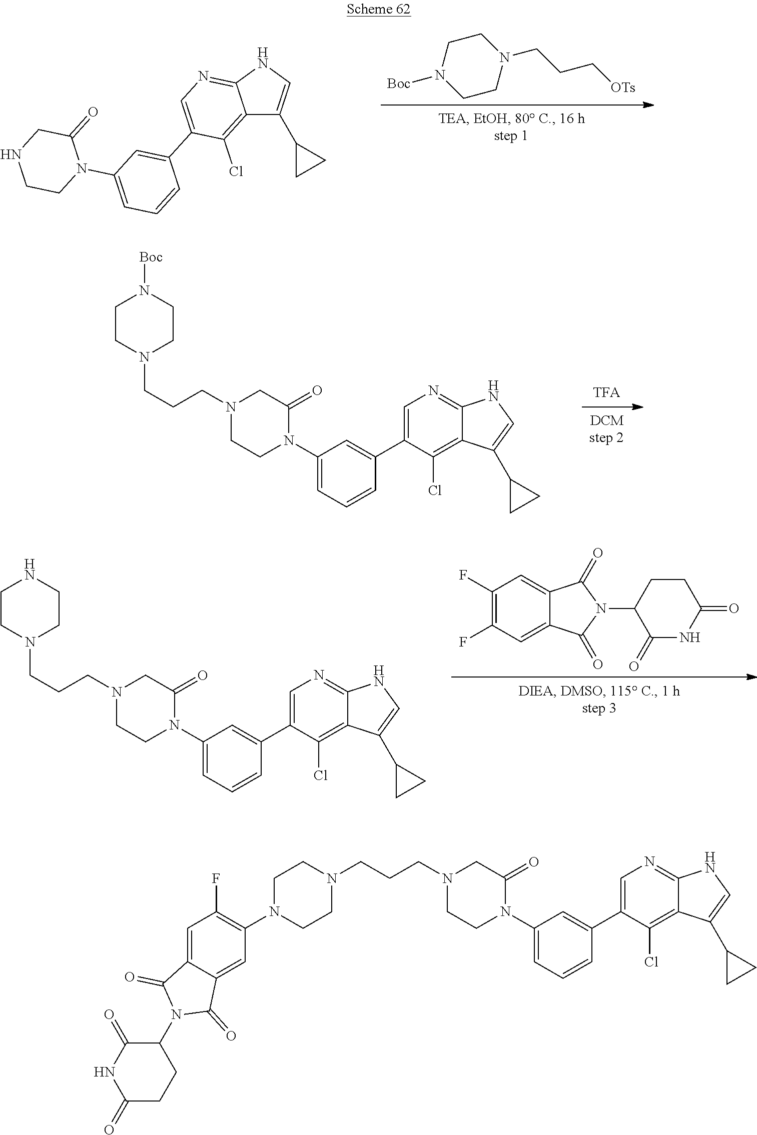

Step 1. Preparation of tert-butyl (4-{3-[4-(3-{4-chloro-3-cyclopropyl-1H-pyrrolo[2,3-b]pyridin-3-yl}phenyl)-3-oxopiperazin-1-yl]propyl}piperazin-1-yl) formate: Following General step G2, the product (50 mg, 28%) was obtained as a yellow solid. Mass (m/z): 593.3 [M+H]⁺.

Step 2. Preparation of 1-(3-{4-chloro-3-cyclopropyl-1H-pyrrolo[2,3-b]pyridin-3-yl}phenyl)-4-[3-(piperazin-1-yl) propyl]piperazin-2-one: Following General step B2, the product (450 mg, 98%) was obtained as a brown solid. Mass (m/z): 492.9 [M+H]⁺.

Step 3. Preparation of 5-(4-{3-[4-(3-{4-chloro-3-cyclopropyl-1H-pyrrolo[2,3-b]pyridin-3-yl}phenyl)-3-oxopiperazin-1-yl]propyl}piperazin-1-yl)-2-(2,6-dioxo piperidin-3-yl)-6-fluoroisoindole-1,3-dione: Following General step L, the product (137 mg, 19%) was obtained as a yellow solid. Mass (m/z): 766.7 [M+H]⁺. ¹H NMR (400 MHZ, DMSO-d₆) δ 11.76 (d, J=2.4 Hz, 1H), 11.12 (s, 1H), 8.13 (d, J=6.2 Hz, 1H), 7.73 (d, J=11.4 Hz, 1H), 7.52-7.45 (m, 3H), 7.42-7.37 (m, 2H), 7.31 (d, J=1.6 Hz, 1H), 5.11 (dd, J=12.8, 5.4 Hz, 1H), 3.75-3.73 (m, 2H), 3.26 (s, 5H), 3.20 (s, 2H), 2.93-2.88 (m, 1H), 2.81-2.79 (m, 2H), 2.59 (s, 4H), 2.54 (s, 3H), 2.45 (d, J=7.0 Hz, 2H), 2.23-2.17 (m, 1H), 2.07-1.98 (m, 1H), 1.73-1.66 (m, 2H), 0.87-0.82 (m, 2H), 0.66-0.62 (m, 2H).

### Example 44: 3-ethyl-1H-pyrrolo 5-(4-((2-(4-(3-(4-chloro-3-ethyl-1H-pyrrolo[2,3-b]pyridin-5-yl)-4-fluorophenyl)-3-oxopiperazin-1-yl)-2-oxoethyl)(methyl)amino)piperidin-1-yl)-2-(2,6-dioxopiperidin-3-yl)-6-fluoroisoindoline-1,3-dione

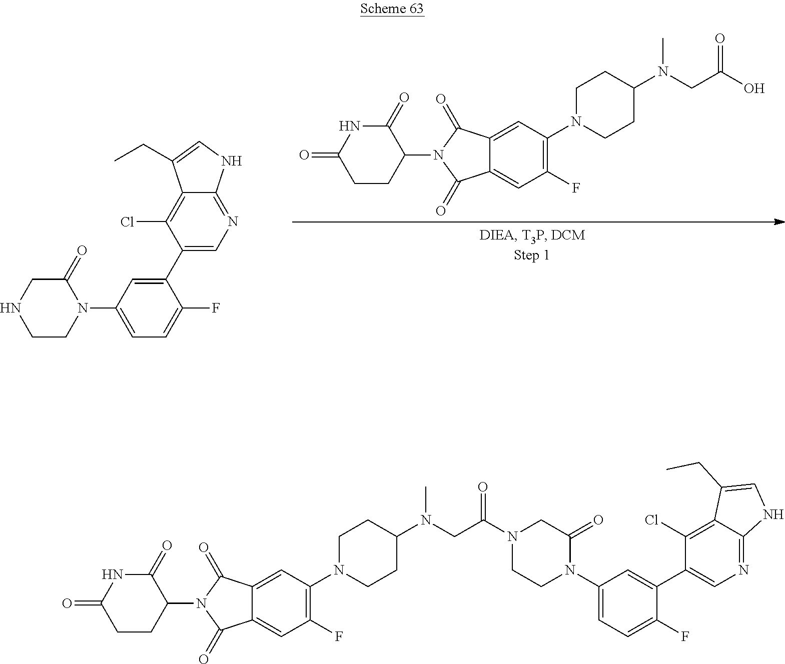 [SMILES: CCc1c[nH]c2ncc(-c3cc(N4CCNCC4=O)ccc3F)c(Cl)c12 ; CN(CC(=O)O)C1CCN(c2cc3c(cc2F)C(=O)N(C2CCC(=O)NC2=O)C3=O)CC1 ; CCc1c[nH]c2ncc(-c3cc(N4CCN(C(=O)CN(C)C5CCN(c6cc7c(cc6F)C(=O)N(C6CCC(=O)NC6=O)C7=O)CC5)CC4=O)ccc3F)c(Cl)c12]

Step 1. Preparation of 5-[4-({2-[4-(3-{4-chloro-3-ethyl-1H-pyrrolo[2,3-b]pyridin-3-yl}-4-fluorophenyl)-3-oxopiperazin-1-yl]-2-oxoethyl}(methyl)amino)piperidin-1-yl]-2-(2,6-dioxo piperidin-3-yl)-6-fluoroisoindole-1,3-dione: Following General step F, the product (40 mg, 18%) was obtained as a yellow solid. Mass (m/z): 801.1 [M+H]⁺. ¹H NMR (400 MHZ, DMSO-d₆) δ 11.88 (s, 1H), 11.13 (s, 1H), 9.57 (s, 1H), 8.11 (d, J=1.8 Hz, 1H), 7.77 (d, J=11.2 Hz, 1H), 7.47 (m, 4H), 5.11 (dd, J=12.8, 5.4 Hz, 1H), 4.49 (d, J=16.2 Hz, 1H), 4.28 (t, J=14.8 Hz, 3H), 3.83 (m, 8H), 2.91 (m, 9H), 2.13 (d, J=18.0 Hz, 2H), 2.03 (m, 1H), 1.90 (d, J=11.2 Hz, 2H), 1.27 (t, J=7.4 Hz, 3H).

### Example 45: 3-cyclopropyl-1H-pyrrolo 5-(4-(2-(4-(3-(4-chloro-3-cyclopropyl-1H-pyrrolo[2,3-b]pyridin-5-yl)phenyl)-3-oxopiperazin-1-yl)ethyl)piperidin-1-yl)-2-(2,6-dioxopiperidin-3-yl)-6-fluoroisoindoline-1,3-dione

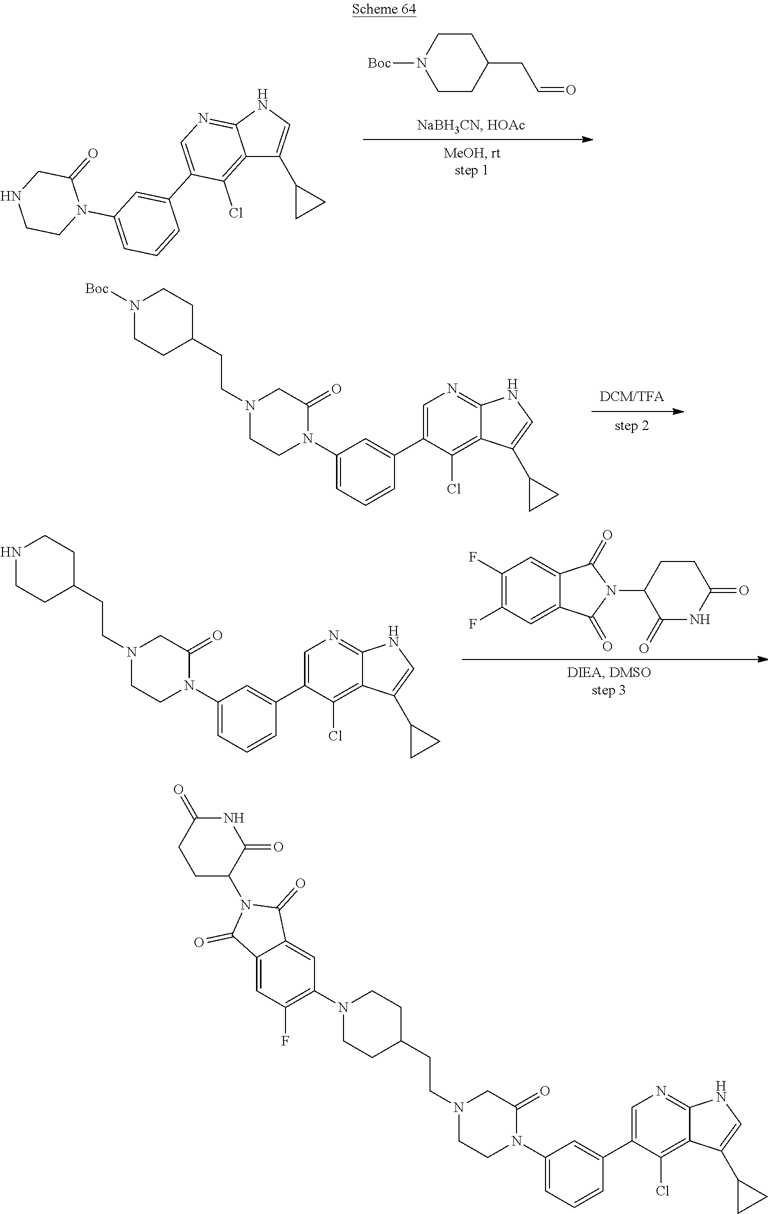

Step 1. Preparation of Tert-butyl (4-{2-[4-(3-{4-chloro-3-cyclopropyl-1H-pyrrolo[2,3-b]pyridin-3-yl}phenyl)-3-oxopiperazin-1-yl]ethyl}piperidin-1-yl) formate: To a mixture of 1-(3-{4-chloro-3-cyclopropyl-1H-pyrrolo[2,3-b]pyridin-3-yl}phenyl)piperazin-2-one (250 mg, 0.682 mmol) in MeOH (10 mL) and HOAc (0.05 mL) was added tert-butyl [4-(2-oxoethyl)piperidin-1-yl]formate (156 mg, 0.682 mmol) and NaBH₃CN (128 mg, 2.04 mmol). The reaction was stirred at 25° C. for 2 hrs. The reaction mixture was concentrated under reduced pressure. The residue was purified by flash column (DCM/MeOH=0˜2.5%) to give the product tert-butyl (4-{2-[4-(3-{4-chloro-3-cyclopropyl-1H-pyrrolo[2,3-b]pyridin-3-yl}phenyl)-3-oxopiperazin-1-yl] ethyl}piperidin-1-yl) formate (370 mg, 89%) as a white solid. Mass (m/z): 577.9 [M+H]⁺.

Step 2. Preparation of 1-(3-{4-chloro-3-cyclopropyl-1H-pyrrolo[2,3-b]pyridin-3-yl}phenyl)-4-[2-(piperidin-4-yl)ethyl]piperazin-2-one: Following General step B2, the product (900 mg, 88%) was obtained as brown oil. Mass (m/z): 477.9 [M+H]⁺.

Step 3. Preparation of 5-(4-{2-[4-(3-{4-chloro-3-cyclopropyl-1H-pyrrolo[2,3-b] pyridin-3-yl}phenyl)-3-oxopiperazin-1-yl]ethyl}piperidin-1-yl)-2-(2,6-dioxopiperidin-3-yl)-6-fluoroisoindole-1,3-dione: Following General step L, the desired product (188 mg, 15%) was obtained as a yellow solid. Mass (m/z): 751.7 [M+H]⁺. ¹H NMR (400 MHZ, DMSO-d₆) δ 11.76 (d, J=2.2 Hz, 1H), 11.12 (s, 1H), 8.13 (d, J=4.4 Hz, 1H), 7.71 (d, J=11.4 Hz, 1H), 7.51-7.38 (m, 5H), 7.31 (d, J=1.8 Hz, 1H), 5.10 (dd, J=12.8, 5.4 Hz, 1H), 3.74 (s, 2H), 3.60 (d, J=12.0 Hz, 2H), 3.20 (s, 2H), 2.86 (dd, J=28.4, 15.2 Hz, 5H), 2.59 (dd, J=36.8, 17.8 Hz, 3H), 2.22-2.18 (m, 1H), 2.09-2.01 (m, 2H), 1.82 (d, J=12.2 Hz, 2H), 1.50 (s, 2H), 1.37-1.31 (m, 2H), 1.23 (s, 1H), 0.87-0.83 (m, 2H), 0.66-0.62 (m, 2H).

### Example 46: 3-cyclopropyl-1H-pyrrolo 3-(6-(4-(2-(4-(3-(4-chloro-3-cyclopropyl-1H-pyrrolo[2,3-b]pyridin-5-yl)phenyl)-3-oxopiperazin-1-yl)-2-oxoethoxy)piperidin-1-yl)-1-oxoisoindolin-2-yl)piperidine-2,6-dione

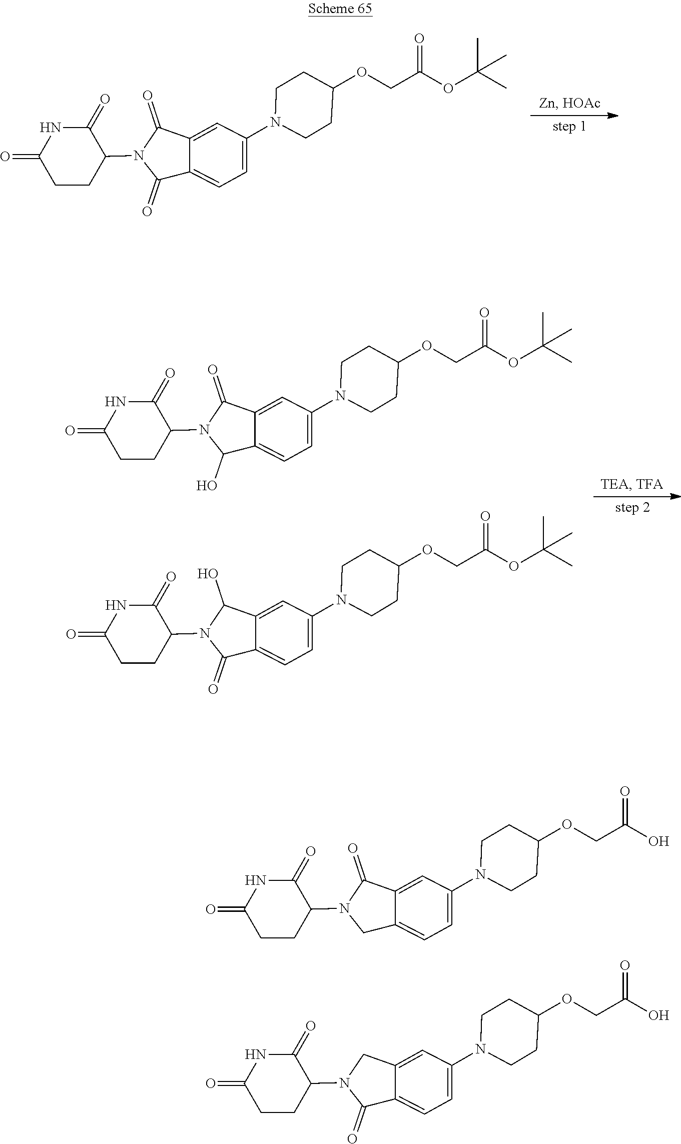 [SMILES: CC(C)(C)OC(=O)COC1CCN(c2ccc3c(c2)C(=O)N(C2CCC(=O)NC2=O)C3=O)CC1 ; CC(C)(C)OC(=O)COC1CCN(c2ccc3c(c2)C(=O)N(C2CCC(=O)NC2=O)C3O)CC1 ; CC(C)(C)OC(=O)COC1CCN(c2ccc3c(c2)C(O)N(C2CCC(=O)NC2=O)C3=O)CC1 ; O=C(O)COC1CCN(c2ccc3c(c2)C(=O)N(C2CCC(=O)NC2=O)C3)CC1 ; O=C(O)COC1CCN(c2ccc3c(c2)CN(C2CCC(=O)NC2=O)C3=O)CC1]

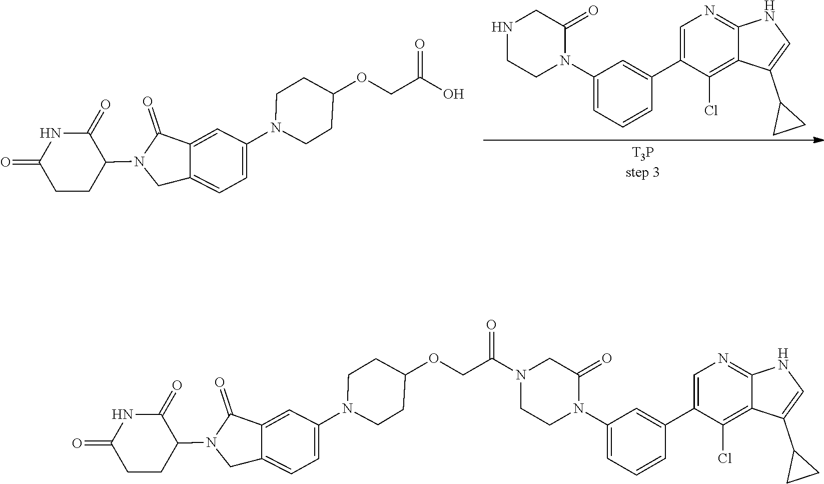 [SMILES: O=C(O)COC1CCN(c2ccc3c(c2)C(=O)N(C2CCC(=O)NC2=O)C3)CC1 ; O=C1CNCCN1c1cccc(-c2cnc3[nH]cc(C4CC4)c3c2Cl)c1 ; O=C1CCC(N2Cc3ccc(N4CCC(OCC(=O)N5CCN(c6cccc(-c7cnc8[nH]cc(C9CC9)c8c7Cl)c6)C(=O)C5)CC4)cc3C2=O)C(=O)N1]

Step 1. Preparation of tert-butyl 2-({1-[2-(2,6-dioxopiperidin-3-yl)-1-hydroxy-3-oxo-1H-isoindol-5-yl]piperidin-4-yl}oxy)acetate and tert-butyl 2-({1-[2-(2,6-dioxo piperidin-3-yl)-3-hydroxy-1-oxo-3H-isoindol-5-yl]piperidin-4-yl}oxy)acetate: To a mixture of tert-butyl 2-({1-[2-(2,6-dioxopiperidin-3-yl)-1,3-dioxoisoindol-5-yl]piperidin-4-yl}oxy)acetate (500 mg, 1.06 mmol) in HOAc (10 mL) was added Zn (694 mg, 10.6 mmol). The reaction mixture was stirred at 90° C. for 16 hrs. The reaction mixture was filtered and the filtrate was concentrated under reduced pressure to give a mixture of tert-butyl 2-({1-[2-(2,6-dioxopiperidin-3-yl)-1-hydroxy-3-oxo-1H-isoindol-5-yl]piperidin-4-yl}oxy)acetate and tert-butyl 2-({1-[2-(2,6-dioxopiperidin-3-yl)-3-hydroxy-1-oxo-3H-isoindol-5-yl]piperidin-4-yl}oxy)acetate as yellow oil (1.0 g, 99%). Mass (m/z): 401.9 [M−55]⁺.

Step 2. Preparation of ({1-[2-(2,6-dioxopiperidin-3-yl)-3-oxo-1H-isoindol-5-yl]piperidin-4-yl}oxy) acetic acid and ({1-[2-(2,6-dioxopiperidin-3-yl)-1-oxo-3H-isoindol-5-yl]piperidin-4-yl}oxy) acetic acid: To a mixture of tert-butyl 2-({1-[2-(2,6-dioxopiperidin-3-yl)-1-hydroxy-3-oxo-1H-isoindol-5-yl]piperidin-4-yl}oxy)acetate and tert-butyl 2-({1-[2-(2,6-dioxo piperidin-3-yl)-3-hydroxy-1-oxo-3H-isoindol-5-yl]piperidin-4-yl}oxy)acetate (1.0 g, 2.11 mmol) in HOAc (10 mL) was added TFA (16.6 g, 146 mmol) and Et₃SiH (5.79 g, 50 mmol). The reaction mixture was stirred at 70° C. for 3 hrs. The reaction mixture was concentrated under reduced pressure. The residue was purified by Combiflash [Gemini-C₁₈ 150×21.2 mm, 5 um; Mobile phase: MeCN/H₂O (0.5% FA); Ratio: 10-20] to give ({1-[2-(2,6-dioxopiperidin-3-yl)-1-oxo-3H-isoindol-5-yl]piperidin-4-yl}oxy) acetic acid (100 mg, 11%), ¹H NMR (400 MHZ, DMSO-d₆) δ 10.95 (s, 1H), 7.50 (d, J=8.5 Hz, 1H), 7.06 (dd, J=10.5, 1.6 Hz, 2H), 5.04 (dd, J=13.3, 5.1 Hz, 1H), 4.33 (dd, J=17.2, 6.2 Hz, 1H), 4.21-4.17 (m, 1H), 4.07 (s, 2H), 3.68-3.55 (m, 3H), 3.03-3.09 (m, 2H), 2.95-2.85 (m, 1H), 2.58 (dd, J=14.2, 2.4 Hz, 1H), 2.42-2.30 (m, 1H), 1.99-1.91 (m, 3H), 1.57-1.48 (m, 2H) and {1-[2-(2,6-dioxopiperidin-3-yl)-3-oxo-1H-isoindol-5-yl]piperidin-4-yl}oxy) acetic acid (100 mg, 11%). Mass (m/z): 401.9 [M+H]⁺. ¹H NMR (400 MHZ, DMSO-d₆) δ 12.58 (s, 1H), 10.98 (s, 1H), 7.41 (d, J=8.4 Hz, 1H), 7.27 (dd, J=8.5, 2.3 Hz, 1H), 7.16 (d, J=2.1 Hz, 1H), 5.09 (dd, J=13.3, 4.9 Hz, 1H), 4.26 (dd, J=52.1, 16.7 Hz, 2H), 4.07 (s, 2H), 3.58-3.55 (m, 3H), 3.97-2.86 (m, 3H), 2.59 (d, J=18.0 Hz, 1H), 2.37 (dt, J=14.0, 9.1 Hz, 1H), 2.00-1.91 (m, 3H), 1.59-1.53 (m, 2H).

Step 3. Preparation of 3-[6-(4-{2-[4-(3-{4-chloro-3-cyclopropyl-1H-pyrrolo[2,3-b]pyridin-3-yl}phenyl)-3-oxopiperazin-1-yl]-2-oxoethoxy}piperidin-1-yl)-1-oxo-3H-isoindol-2-yl]piperidine-2,6-dione: Following General step F, the desired product was obtained as white solid (34 mg, 45%). Mass (m/z): 749.7 [M+H]⁺. ¹H NMR (400 MHZ, CDCl₃) δ 13.06 (s, 1H), 8.25 (d, J=13.0 Hz, 1H), 8.12 (s, 1H), 7.96-7.83 (m, 2H), 7.61-7.57 (m, 1H), 7.54 (d, J=6.8 Hz, 1H), 7.46-7.42 (m, 2H), 7.39 (d, J=7.2 Hz, 1H), 7.21 (s, 1H), 5.19 (dd, J=12.8, 4.2 Hz, 1H), 4.53-4.41 (m, 4H), 4.33 (s, 2H), 4.04 (s, 1H), 3.95-3.85 (m, 4H), 3.77-3.72 (m, 2H), 3.39 (s, 2H), 2.96-2.81 (m, 2H), 2.43-2.37 (m, 3H), 2.24-2.19 (m, 2H), 2.13 (d, J=12.2 Hz, 2H), 1.00 (q, J=6.0 Hz, 2H), 0.69 (q, J=5.4 Hz, 2H).

### Example 47: 3-cyclopropyl-1H-pyrrolo 3-(5-(4-(2-(4-(3-(4-chloro-3-cyclopropyl-1H-pyrrolo[2,3-b]pyridin-5-yl)phenyl)-3-oxopiperazin-1-yl)-2-oxoethoxy)piperidin-1-yl)-1-oxoisoindolin-2-yl)piperidine-2,6-dione

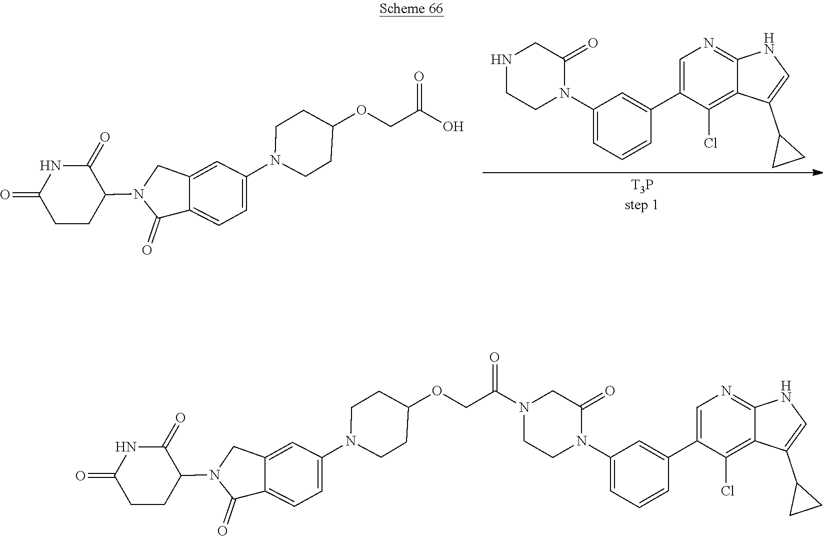 [SMILES: O=C(O)COC1CCN(c2ccc3c(c2)CN(C2CCC(=O)NC2=O)C3=O)CC1 ; O=C1CNCCN1c1cccc(-c2cnc3[nH]cc(C4CC4)c3c2Cl)c1 ; O=C1CCC(N2Cc3cc(N4CCC(OCC(=O)N5CCN(c6cccc(-c7cnc8[nH]cc(C9CC9)c8c7Cl)c6)C(=O)C5)CC4)ccc3C2=O)C(=O)N1]

Step 1. Preparation of 3-[5-(4-{2-[4-(3-{4-chloro-3-cyclopropyl-1H-pyrrolo[2,3-b]pyridin-3-yl}phenyl)-3-oxopiperazin-1-yl]-2-oxoethoxy}piperidin-1-yl)-1-oxo-3H-isoindol-2-yl]piperidine-2,6-dione: Following General step F, the product was obtained as a white solid. (27 mg, 28%). Mass (m/z): 749.7 [M+H]⁺. ¹H NMR (400 MHZ, CD₃OD) δ 8.16 (s, 1H), 7.65 (dd, J=13.6, 8.2 Hz, 1H), 7.56 (t, J=7.8 Hz, 1H), 7.50-7.46 (m, 2H), 7.41 (d, J=7.8 Hz, 1H), 7.16-7.21 (m, 3H), 5.11 (dd, J=13.2, 5.2 Hz, 1H), 4.45 (s, 1H), 4.39 (dd, J=10.8, 4.2 Hz, 4H), 4.05-3.98 (m, 2H), 3.95 (s, 1H), 3.87 (t, J=5.2 Hz, 1H), 3.75 (dd, J=6.6, 3.4 Hz, 3H), 3.24 (d, J=6.8 Hz, 3H), 2.94-2.85 (m, 1H), 2.79-2.74 (m, 1H), 2.45 (m, 1H), 2.24-2.19 (m, 1H), 2.17-2.08 (m, 3H), 1.84 (s, 2H), 0.91 (d, J=7.8 Hz, 2H), 0.65 (d, J=5.2 Hz, 2H).

### Example 48: 3-cyclopropyl-1H-pyrrolo 5-(4-((1-(4-(3-(4-chloro-3-cyclopropyl-1H-pyrrolo[2,3-b]pyridin-5-yl)phenyl)-3-oxopiperazin-1-yl)-1-oxopropan-2-yl)oxy)piperidin-1-yl)-2-(2,6-dioxopiperidin-3-yl)-6-fluoroisoindoline-1,3-dione

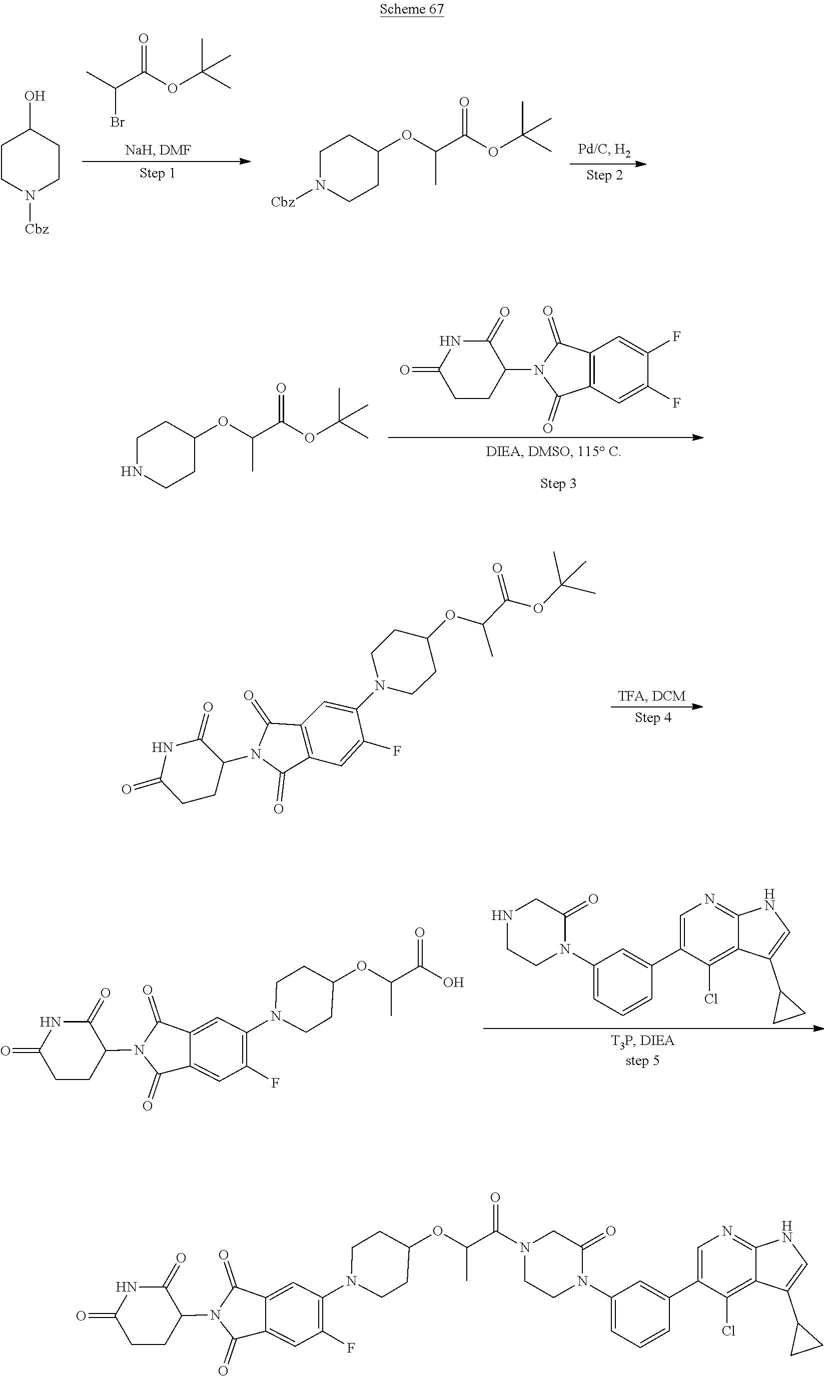

Step 1. Preparation of benzyl 4-4-{[1-(tert-butoxy)-1-oxopropan-2-yl]oxy}piperidine-1-carboxylate: To a solution of benzyl 4-4-hydroxypiperidine-1-carboxylate (10.2 g, 43.4 mmol), in DMF (100 mL) at 0° C. was added NaH (2.26 g, 56.42 mmol). The mixture was stirred under nitrogen at 25° C. for 1 h. Then added tert-butyl 2-bromopropanoate (13.61 g, 65.1 mmol). The mixture was stirred under nitrogen at 110° C. for 16 hrs. cooled to room temperature, quenched with aqueous NH₄Cl, diluted with H₂O (500 mL), extracted with EA (500 mL×2), washed with water (50 mL×2) and saturated brine. Organic layer was concentrated under vacuum, the residue was purified by Combiflash (PE/EA=4:1) to give the product (5 g, 31.8%) as yellowish oil. Mass (m/z): 386.2 [M+Na]⁺.

Step 2. Preparation of tert-butyl 2-(piperidin-4-yloxy) propanoate: Following General step K, the desired product (500 mg, 71.43%) was obtained as light colored oil. Mass (m/z): 230.2 [M+H]⁺.

Step 3. Preparation of tert-butyl 2-({1-[2-(2,6-dioxopiperidin-3-yl)-6-fluoro-1,3-dioxoisoindol-5-yl]piperidin-4-yl}oxy) propanoate: Following General step L, the product (1 g, 71%) was obtained as yellow solid. Mass (m/z): 504.2 [M+H]⁺.

Step 4. Preparation of 2-({1-[2-(2,6-dioxopiperidin-3-yl)-6-fluoro-1,3-dioxoisoindol-5-yl]piperidin-4-yl}oxy) propanoic acid: Following General step B2, the desired product (800 mg, 80%) was obtained as yellow solid. Mass (m/z): 447.8 [M+H]⁺.

Step 5. Preparation of 5-[4-({1-[4-(3-{4-chloro-3-cyclopropyl-1H-pyrrolo[2,3-b]pyridin-3-yl}phenyl)-3-oxopiperazin-1-yl]-1-oxopropan-2-yl}oxy)piperidin-1-yl]-2-(2,6-dioxopiperidin-3-yl)-6-fluoroisoindole-1,3-dione: Following General step F, the desired product was obtained as yellow solid (60 mg, 39.58%). Mass (m/z): 796.3 [M+H]⁺. ¹H NMR (400 MHZ, DMSO-d₆) δ 11.75 (d, J=9.9 Hz, 1H), 11.11 (s, 1H), 8.12 (d, J=4.3 Hz, 1H), 7.64 (dd, J=36.6, 11.3 Hz, 1H), 7.52 (t, J=7.8 Hz, 2H), 7.42 (dd, J=18.9, 7.2 Hz, 3H), 7.29 (d, J=9.8 Hz, 1H), 5.10 (dd, J=12.8, 5.4 Hz, 1H), 4.56 (m, 2H), 4.21 (d, J=5.9 Hz, 1H), 3.80 (s, 2H), 3.61 (s, 1H), 3.47 (s, 2H), 3.04 (t, J=9.7 Hz, 2H), 2.88 (m, 1H), 2.57 (m, 4H), 2.14 (m, 2H), 2.00 (d, J=8.6 Hz, 2H), 1.62 (d, J=9.2 Hz, 2H), 1.31 (d, J=6.1 Hz, 3H), 0.83 (s, 2H), 0.63 (d, J=4.5 Hz, 2H).

### Example 49: 5-(4-(2-(4-(3-(4-chloro-3-cyclopropyl-1H-pyrrolo[2,3-b]pyridin-5-yl)phenyl)-3-oxopiperazin-1-yl) propoxy)piperidin-1-yl)-2-(2,6-dioxopiperidin-3-yl)-6-fluoroisoindoline-1,3-dione

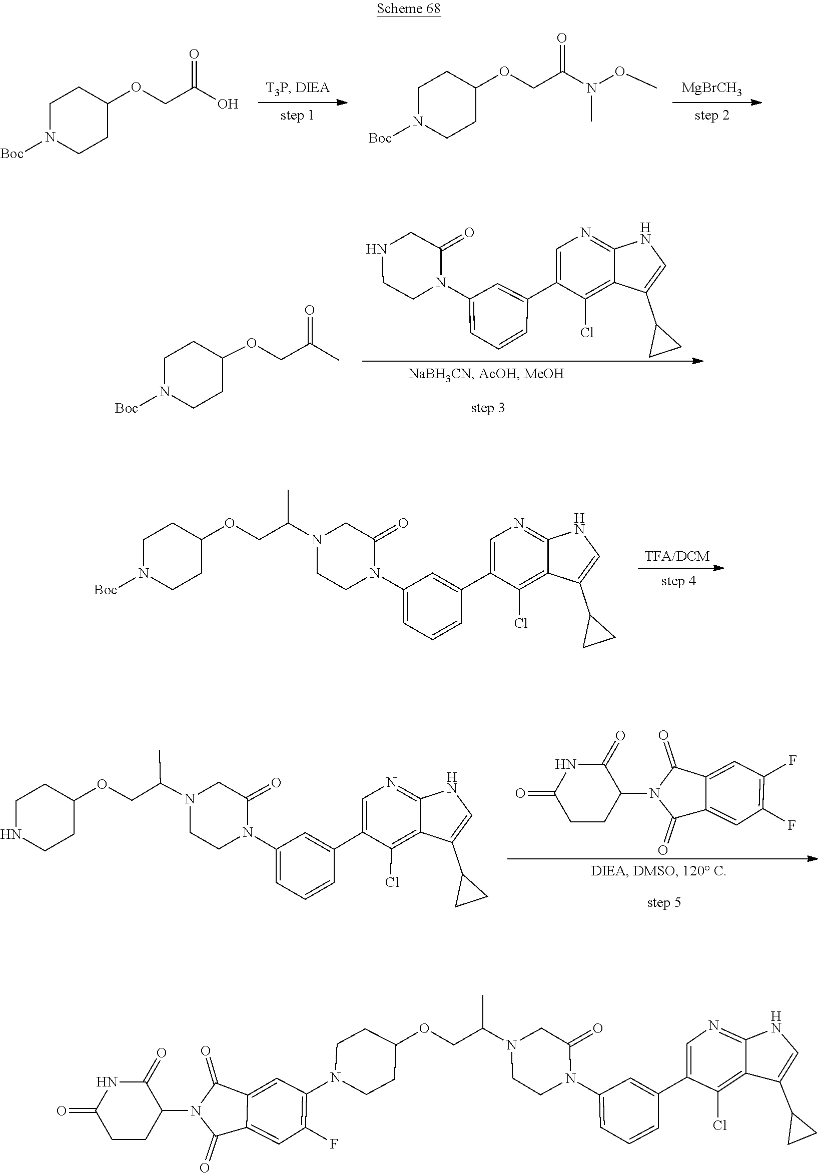

Step 1. Preparation of tert-butyl 4-(2-(methoxy(methyl)amino)-2-oxoethoxy)piperidine-1-carboxylate: To a solution of {[1-(tert-butyl-$1\hat{ }{3}-oxy)piperidin-4-yl]oxy}acetic acid (500 mg, 1.9208 mmol) and methoxy(methyl)amine hydrochloride (281 mg, 2.88 mmol) in DCM (10 mL) was added DIEA (993 mg, 7.68 mmol) and T3P (2445 mg, 3.84 mmol). The reaction mixture was stirred at 25° C. for 16 hrs. The reaction mixture was washed with water (20 mL) and extracted with EA (20 mL). The organic phase was collected and evaporated to give the product as a colorless oil (600 mg, 92%). Mass (m/z): 247.1 [M−55]⁺.

Step 2. Preparation of tert-butyl 4-(2-oxopropoxy)piperidine-1-carboxylate: To a solution of tert-butyl (4-{[methoxy(methyl) carbamoyl]methoxy}piperidin-1-yl) formate (600 mg, 1.9778 mmol) in THF (5 mL) was added methyl magnesium bromide (0.8 mL, 2.37 mmol) at −78° C. under N₂. The reaction mixture was stirred at −78° C. for 1 hr and 0° C. for 1 hr. The reaction mixture was quenched by saturated aqueous NH₄Cl. The reaction mixture was washed with water (10 mL) and extracted with EA (10 mL). The organic phase was collected and evaporated to give the product as a colorless oil (300 mg, 52%). Mass (m/z): 202.1 [M−55]⁺.

Step 3. Preparation of tert-butyl 4-(2-(4-(3-(4-chloro-3-cyclopropyl-1H-pyrrolo[2,3-b]pyridin-5-yl)phenyl)-3-oxopiperazin-1-yl) propoxy)piperidine-1-carboxylate: Following General step M1, the product was obtained as a colorless oil (170 mg, 46%). Mass (m/z): 607.9 [M+H]⁺.

Step 4. Preparation of 1-(3-(4-chloro-3-cyclopropyl-1H-pyrrolo[2,3-b]pyridin-5-yl)phenyl)-4-(1-(piperidin-4-yloxy) propan-2-yl)piperazin-2-one: Following General step B2, the product was obtained as a yellow oil (200 mg, 98%). Mass (m/z): 508.0 [M+H]⁺.

Step 5. Preparation of 5-(4-(2-(4-(3-(4-chloro-3-cyclopropyl-1H-pyrrolo[2,3-b]pyridin-5-yl)phenyl)-3-oxopiperazin-1-yl) propoxy)piperidin-1-yl)-2-(2,6-dioxopiperidin-3-yl)-6-fluoroisoindoline-1,3-dione: Following General step L, the product as a yellow solid (40 mg, 12%). Mass (m/z): 782.2 [M+H]⁺. ¹H NMR (400 MHZ, DMSO-d₆) δ 11.76 (d, J=2.2 Hz, 1H), 11.08 (s, 1H), 8.09 (s, 1H), 7.69 (d, J=11.4 Hz, 1H), 7.54 (s, 1H), 7.49-7.35 (m, 4H), 7.29 (d, J=1.7 Hz, 1H), 5.07 (dd, J=12.8, 5.4 Hz, 1H), 4.01-3.92 (m, 3H), 3.73-3.58 (m, 8H), 3.50-3.44 (m, 2H), 3.05 (t, J=9.6 Hz, 2H), 2.91-2.78 (m, 1H), 2.61-2.50 (m, 1H), 2.22-2.09 (m, 1H), 2.04-1.93 (m, 3H), 1.72-1.56 (m, 2H), 1.31 (d, J=5.9 Hz, 3H), 0.84-0.78 (m, 2H), 0.64-0.57 (m, 2H).

### Example 50: 1-(5-(4-(3-(4-(3-(4-chloro-3-ethyl-1H-pyrrolo[2,3-b]pyridin-5-yl)phenyl)-3-oxopiperazin-1-yl)-3-oxopropyl)piperazine-1-carbonyl)-2-methylphenyl)dihydro pyrimidine-2,4 (1H,3H)-dione

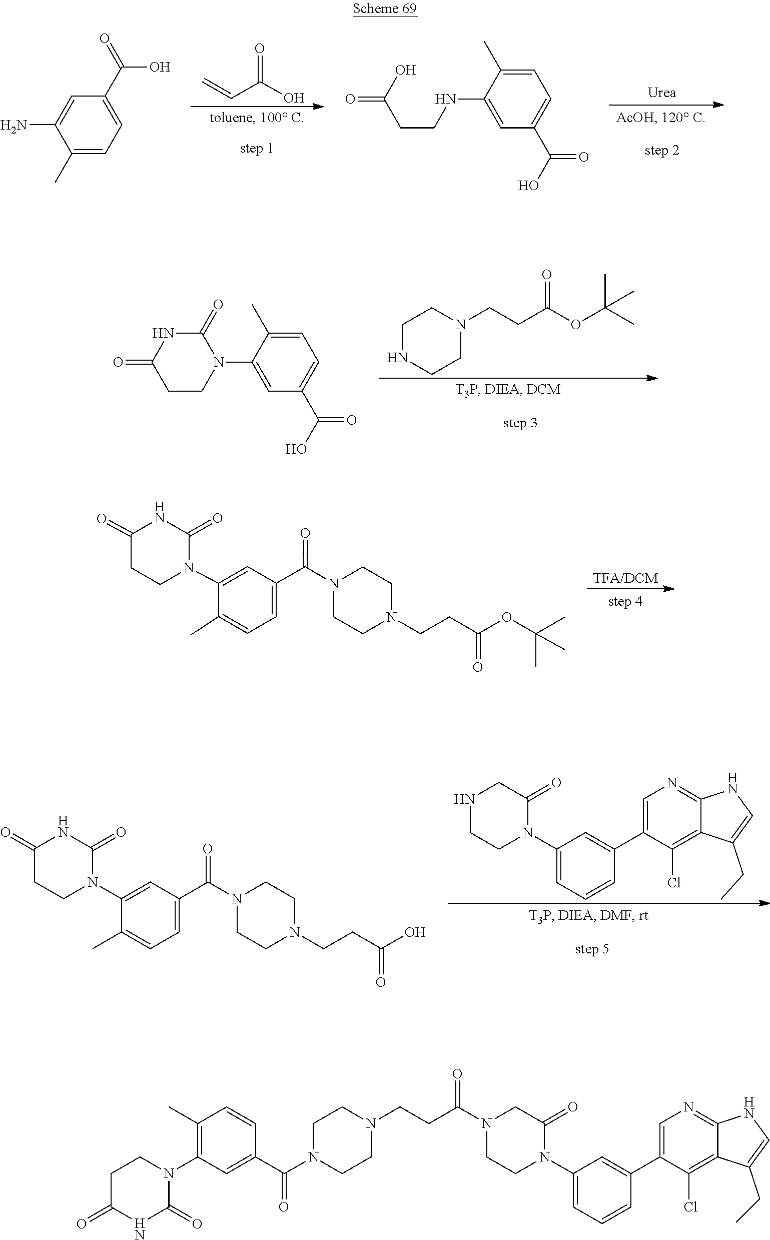

Step 1. Preparation of 3-((2-carboxyethyl)amino)-4-methylbenzoic acid: To a solution 3-amino-4-methylbenzoic acid (1.1 g, 7.28 mmol) in toluene (20 mL) was added acrylic acid (2.09 g, 29.11 mmol). The reaction mixture was stirred at 100° C. under N₂ for 3 hrs. The reaction mixture was filtered, the filter cake was concentrated under vacuum to give the product 3-((2-carboxyethyl)amino)-4-methylbenzoic acid as white solid (1.5 g, 92%). Mass (m/z): 224.1 [M+Na].

Step 2. Preparation of 3-(2,4-dioxotetrahydropyrimidin-1 (2H)-yl)-4-methylbenzoic acid: To a solution of 3-((2-carboxyethyl)amino)-4-methylbenzoic acid (1.5 g, 6.72 mmol) in AcOH (30 mL) was added Urea (1 g, 16.80 mmol). The reaction mixture was stirred at 120° C. under N₂ for 16 hrs. The solvent was removed under reduced pressure to give the product 3-(2,4-dioxotetrahydropyrimidin-1 (2H)-yl)-4-methylbenzoic acid as brown oil (4.1 g, 73%). Mass (m/z): 249.0 [M+H]⁺.

Step 3. Preparation of tert-butyl 3-(4-(3-(2,4-dioxotetrahydropyrimidin-1 (2H)-yl)-4-methylbenzoyl)piperazin-1-yl) propanoate: From 3-(2,4-dioxotetrahydropyrimidin-1 (2H)-yl)-4-methylbenzoic acid (700 mg, 2.82 mmol), Following General step F, the product tert-butyl 3-(4-(3-(2,4-dioxotetrahydropyrimidin-1 (2H)-yl)-4-methyl benzoyl)piperazin-1-yl) propanoate was obtained as yellow solid (1.2 g, 95%). Mass (m/z): 445.2 [M+Na]⁺.

Step 4. Preparation of 3-(4-(3-(2,4-dioxotetrahydropyrimidin-1 (2H)-yl)-4-methyl benzoyl)piperazin-1-yl) propanoic acid: Following General step B2, the product was obtained as yellow oil (1 g, 76%). Mass (m/z): 389.2 [M+Na]⁺. ¹H NMR (400 MHZ, DMSO-d₆) δ 10.43 (s, 1H), 7.40 (dd, J=4.8, 3.2 Hz, 2H), 7.36 (d, J=1.6 Hz, 1H), 3.82-3.78 (m, 4H), 3.54 (dd, J=8.4, 5.2 Hz, 4H), 3.36-3.31 (m, 4H), 2.76 (t, J=7.4 Hz, 4H), 2.24 (s, 3H).

Step 5. Preparation of 1-(5-(4-(3-(4-(3-(4-chloro-3-ethyl-1H-pyrrolo[2,3-b]pyridin-5-yl)phenyl)-3-oxopiperazin-1-yl)-3-oxopropyl)piperazine-1-carbonyl)-2-methylphenyl)dihydropyrimidine-2,4 (1H,3H)-dione: Following General step F, the product was obtained as a white solid (32 mg, 30%). Mass (m/z): 724.8 [M+H]⁺. ¹H NMR (400 MHZ, DMSO-d₆) δ 11.81 (s, 1H), 10.39 (s, 1H), 8.13 (d, J=5.6 Hz, 1H), 7.55-7.46 (m, 2H), 7.45-7.32 (m, 5H), 7.28 (s, 1H), 4.33 (s, 1H), 4.20 (s, 1H), 3.93-3.74 (m, 6H), 3.63-3.33 (m, 7H), 2.92 (q, J=7.4 Hz, 2H), 2.85-2.56 (m, 7H), 2.22 (s, 3H), 1.27 (t, J=7.4 Hz, 3H).

### Example 51: 5-(4-(3-((4-(3-(4-chloro-3-cyclopropyl-1H-pyrrolo[2,3-b]pyridin-5-yl)phenyl)-3-oxopiperazin-1-yl)methyl)cyclobutyl)piperazin-1-yl)-2-(2,6-dioxopiperidin-3-yl)-6-fluoroisoindoline-1,3-dione

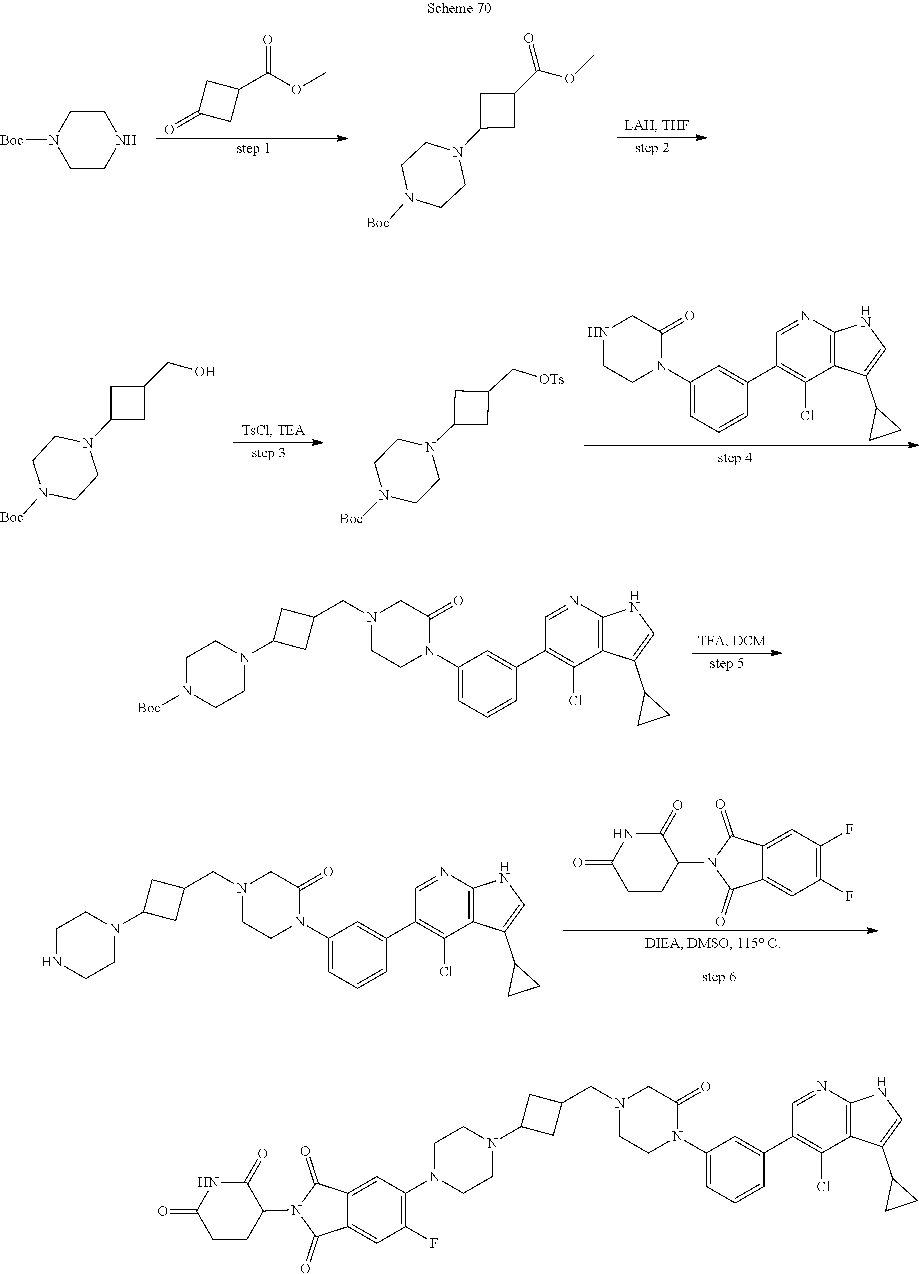

Step 1. Preparation of tert-butyl 4-(3-(methoxycarbonyl)cyclobutyl)piperazine-1-carboxylate: To a solution of tert-butyl piperazine-1-carboxylate (5 g, 26.8 mmol), methoxyacetic acid (3.43 g, 26.8 mmol), in 1,2-dichloroethane (50 mL) was added AcOH (1 mL). The reaction solution was stirred at rt for 8 hrs. Sodium triacetoxyborohydride (17.04 g, 80.4 mmol) was added. The reaction solution was stirred at rt for 18 hrs. concentrated under reduced pressure. The residue was purified by column chromatography (DCM/MeOH=10:1) to give the target product (2.5 g, 29.85%) as a colorless oil. Mass (m/z): 299.1 [M+H]⁺.

Step 2. Preparation of tert-butyl 4-(3-(hydroxymethyl)cyclobutyl)piperazine-1-carboxylate: To a solution of tert-butyl 4-[3-(methoxycarbonyl)cyclobutyl]piperazine-1-carboxylate (2.5 g, 0.0084 mol) in THF (50 mL) was added LiAlH₄ (1.28 g, 0.0336 mol) at 0° C. The reaction mixture was stirred at rt under N₂ for 3 hrs. After the reaction completed, H₂O (1.3 mL), NaOH (15%, 1 mL), H₂O (10 mL) was added to the reaction mixture, MgSO₄ (10 g) was added to the reaction mixture. The reaction mixture was filtered, the filtrate was concentrated under vacuum to give the target product (1 g, 41.67%) as a colorless oil. Mass (m/z): 271 [M+H]⁺.

Step 3. Preparation of tert-butyl 4-(3-((tosyloxy)methyl)cyclobutyl)piperazine-1-carboxylate: To a solution of tert-butyl {4-[3-(hydroxymethyl)cyclobutyl]piperazin-1-yl}formate (1 g, 0.0037 mol) and 4-DMAP (0.5 g, 0.0040 mol) in DCM (20 mL) was added TsCl (0.71 g, 0.0037 mol) at 0° C. under N₂, the reaction mixture was stirred at rt for 18 hrs. The reaction mixture was concentrated under vacuum, the residue was purified by flash column (PE/EA=5:1) to give the desired product (0.7 g, yield: 43.24%) as white solid. Mass (m/z): 425.0 [M+H]⁺.

Step 4. Preparation of tert-butyl 4-(3-((4-(3-(4-chloro-3-cyclopropyl-1H-pyrrolo[2,3-b]pyridin-5-yl)phenyl)-3-oxopiperazin-1-yl)methyl)cyclobutyl)piperazine-1-carboxylate: A mixture solution of 1-(3-{4-chloro-3-cyclopropyl-1H-pyrrolo[2,3-b]pyridin-3-yl}phenyl)piperazin-2-one (300 mg, 0.8178 mmol), tert-butyl {4-[3-({[(4-methyl benzene) sulfonyl]oxy}methyl)cyclobutyl]piperazin-1-yl}formate (452.43 mg, 1.0631 mmol), NaI (122.67 mg, 00.8178 mmol) and K₂CO₃ (339.08 mg, 2.4534 mmol) in DMA (3 mL) was stirred at 120° C. for 18 hrs. Then the residue was diluted with EA (20 mL), washed with water (20 mL×4), dried with Na₂SO₄, filtered and evaporated. The residue was purified by column chromatography to afford target product (200 mg, 56.02%) as a yellow solid. Mass (m/z): 619 [M+H]⁺.

Step 5. Preparation of 1-(3-(4-chloro-3-cyclopropyl-1H-pyrrolo[2,3-b]pyridin-5-yl)phenyl)-4-((3-(piperazin-1-yl)cyclobutyl)methyl)piperazin-2-one: Following General step B2, the desired product (150 mg, yield: 85.12%) was obtained as a black oil. Mass (m/z): 519 [M+H]⁺.

Step 6. Preparation of 5-(4-(3-((4-(3-(4-chloro-3-cyclopropyl-1H-pyrrolo[2,3-b] pyridin-5-yl)phenyl)-3-oxopiperazin-1-yl)methyl)cyclobutyl)piperazin-1-yl)-2-(2,6-dioxopiperidin-3-yl)-6-fluoroisoindoline-1,3-dione: Following General step L, the product (40 mg, yield: 17.8%) was obtained as a yellow solid. Mass (m/z): 793 [M+H]⁺. ¹H NMR (400 MHz, DMSO-d₆) δ 11.77 (d, J=2.4 Hz, 1H), 11.12 (s, 1H), 8.13 (d, J=5.8 Hz, 1H), 7.77 (d, J=10.7 Hz, 1H), 7.57-7.46 (m, 2H), 7.44-7.36 (m, 2H), 7.31 (d, J=1.6 Hz, 1H), 5.12 (dd, J=12.8, 5.4 Hz, 1H), 4.05 (s, 1H), 3.73 (s, 3H), 3.20 (s, 4H), 2.85 (dd, J=19.2, 14.0 Hz, 5H), 2.60 (dd, J=35.0, 16.8 Hz, 6H), 2.24 (ddd, J=22.1, 13.4, 10.7 Hz, 4H), 2.14-1.98 (m, 2H), 1.92 (s, 1H), 1.68 (s, 2H), 0.85 (ddd, J=8.2, 6.0, 4.0 Hz, 2H), 0.68-0.60 (m, 2H).

### Example 52: 3-cyclopropyl-1H-pyrrolo 5-(2-(2-(4-(3-(4-chloro-3-cyclopropyl-1H-pyrrolo[2,3-b]pyridin-5-yl)phenyl)-3-oxopiperazin-1-yl)ethyl)-2,7-diazaspiro[3.5]nonan-7-yl)-2-(2,6-dioxopiperidin-3-yl)-6-fluoroisoindoline-1,3-dione

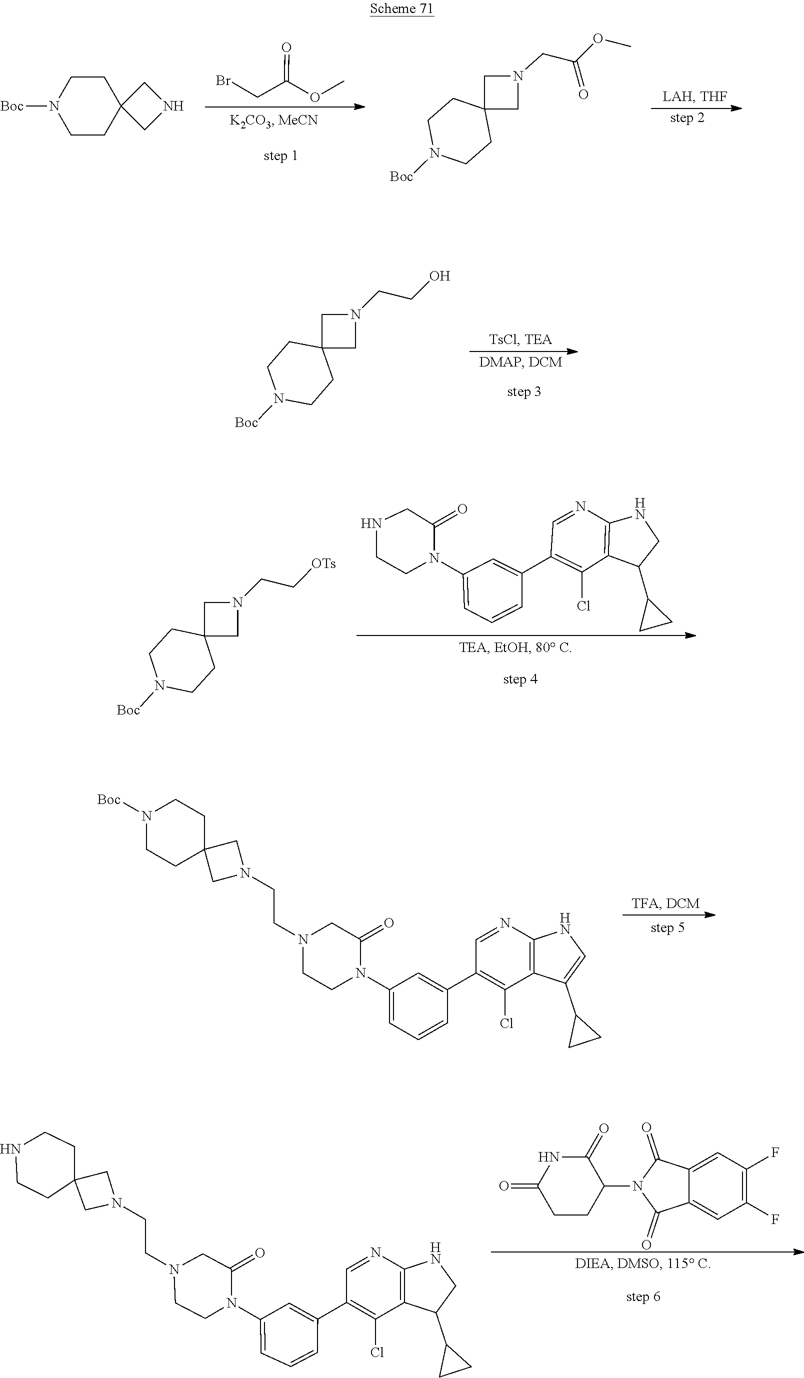

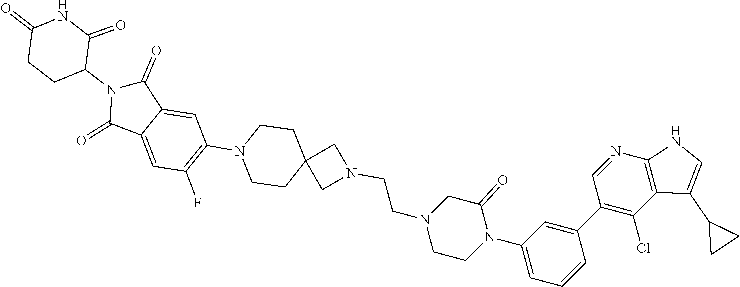 [SMILES: O=C1CCC(N2C(=O)c3cc(F)c(N4CCC5(CC4)CN(CCN4CCN(c6cccc(-c7cnc8[nH]cc(C9CC9)c8c7Cl)c6)C(=O)C4)C5)cc3C2=O)C(=O)N1]

Step 1. Preparation of methyl 2-[7-(tert-butyl-$1\hat{ }{3}-oxy)-2,7-diazaspiro[3.5]nonan-2-yl]acetate: To a solution of tert-butyl {2,7-diazaspiro[3.5]nonan-7-yl}formate (200 mg, 0.88 mmol) in MeCN (10 mL) at 25° C. was added methyl 2-bromoacetate (161.5 mg, 1.056 mmol) and K₂CO₃ (973 mg, 7.04 mmol). The mixture stirred under nitrogen at 85° C. for 1 h. the mixture was concentrated, the residue was purified by Combiflash (PE/EA=1:1) to give the product (180 mg, 61.5%) as colorless oil. Mass (m/z): 299.0 [M+Na]⁺.

Step 2. Preparation of tert-butyl [2-(2-hydroxyethyl)-2,7-diazaspiro[3.5]nonan-7-yl] formate: To a solution of methyl 2-[7-(tert-butyl-$1\hat{ }{3}-oxy)-2, 7-diazaspiro[3.5]nonan-2-yl]acetate (180 mg, 0.60 mmol) in THF (5 mL) was added LiAlH₄ (36 mg, 0.90 mmol) at 0° C. The reaction mixture was stirred at 0° C. for 2 hrs. quenched with aqueous 15% NaOH solution at 0° C., by filtration, the filtrate was concentrated to give the product (90 mg, 52.41%) as yellowish oil. Mass (m/z): 271.2 [M+H]⁺.

Step 3. Preparation of tert-butyl [2-(2-{[(4-methylbenzene) sulfonyl]oxy}ethyl)-2,7-diazaspiro[3.5]nonan-7-yl]formate: To a solution of tert-butyl [2-(2-hydroxyethyl)-2,7-diazaspiro[3.5]nonan-7-yl]formate (90 mg, 0.33 mmol) in DCM (5 mL) was added TsCl (94 mg, 0.50 mmol), TEA (67 mg, 0.66 mmol) and 4-DMAP (8 mg, 0.066 mmol) at 0° C. The reaction mixture was stirred at 25° C. for 5 hrs. The solvent is removed under vacuum, the residue was purified by Combiflash (PE/EA=1:1) to give the product (76 mg, 32.33%) as yellowish oil. Mass (m/z): 425.2 [M+H]⁺.

Step 4. Preparation of tert-butyl (2-{2-[4-(3-{4-chloro-3-cyclopropyl-1H-pyrrolo[2,3-b]pyridin-3-yl}phenyl)-3-oxopiperazin-1-yl]ethyl}-2,7-diazaspiro[3.5]nonan-7-yl) formate: Following General step G2, the product (30 mg, 5.43%) was obtained as brown solid. Mass (m/z): 619.1 [M+H]⁺.

Step 5. Preparation of 1-(3-{4-chloro-3-cyclopropyl-1H-pyrrolo[2,3-b]pyridin-3-yl}phenyl)-4-(2-{2,7-diazaspiro[3.5]nonan-2-yl}ethyl)piperazin-2-one: Following General step B2, the desired product (20 mg, 71%) was obtained as brown solid. Mass (m/z): 518.9 [M+H]⁺.

Step 6. Preparation of 5-(2-{2-[4-(3-{4-chloro-3-cyclopropyl-1H-pyrrolo[2,3-b]pyridin-3-yl}phenyl)-3-oxopiperazin-1-yl]ethyl}-2,7-diazaspiro[3.5]nonan-7-yl)-2-(2,6-dioxopiperidin-3-yl)-6-fluoroisoindole-1,3-dione: Following General step L, the desired product was obtained as yellow solid (2 mg, 6.5%). Mass (m/z): 792.8 [M+H]⁺. ¹HNMR (400 MHZ, DMSO-d₆) δ 11.76 (s, 1H), 11.10 (d, J=9.4 Hz, 1H), 8.13 (d, J=5.4 Hz, 1H), 7.73 (d, J=11.3 Hz, 1H), 7.63 (d, J=12.4 Hz, 1H), 7.52 (m, 1H), 7.47 (s, 1H), 7.40 (d, J=7.5 Hz, 1H), 7.32 (s, 1H), 7.06 (d, J=7.3 Hz, 1H), 5.09 (m, 1H), 3.95 (s, 2H), 3.78 (s, 2H), 3.65 (s, 2H), 3.21 (s, 2H), 2.88 (s, 2H), 2.67 (s, 2H), 2.57 (d, J=31.3 Hz, 4H), 2.33 (s, 1H), 2.16 (d, J=28.2 Hz, 2H), 2.07 (s, 6H), 1.21 (d, J=17.3 Hz, 2H), 0.84 (d, J=7.6 Hz, 2H), 0.65 (m, 2H).

### Example 53: 5-(4-(3-(4-(6-(4-chloro-3-cyclopropyl-1H-pyrrolo[2,3-b]pyridin-5-yl)pyridin-2-yl)-3-oxopiperazin-1-yl) propyl)piperazin-1-yl)-2-(2,6-dioxopiperidin-3-yl)-6-fluoroisoindoline-1,3-dione

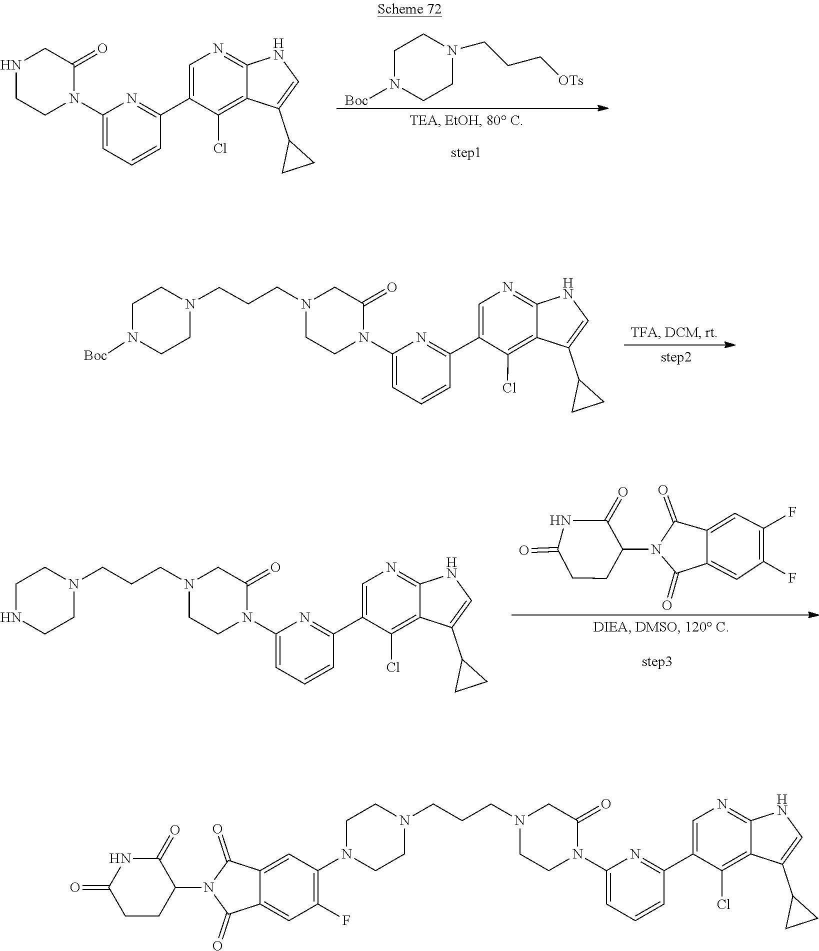 [SMILES: O=C1CNCCN1c1cccc(-c2cnc3[nH]cc(C4CC4)c3c2Cl)n1 ; Cc1ccc(S(=O)(=O)OCCCN2CCN(C(=O)OC(C)(C)C)CC2)cc1 ; CC(C)(C)OC(=O)N1CCN(CCCN2CCN(c3cccc(-c4cnc5[nH]cc(C6CC6)c5c4Cl)n3)C(=O)C2)CC1 ; O=C1CN(CCCN2CCNCC2)CCN1c1cccc(-c2cnc3[nH]cc(C4CC4)c3c2Cl)n1 ; O=C1CCC(N2C(=O)c3cc(F)c(F)cc3C2=O)C(=O)N1 ; O=C1CCC(N2C(=O)c3cc(F)c(N4CCN(CCCN5CCN(c6cccc(-c7cnc8[nH]cc(C9CC9)c8c7Cl)n6)C(=O)C5)CC4)cc3C2=O)C(=O)N1]

Step 1. Preparation of tert-butyl 4-(3-(4-(6-(4-chloro-3-cyclopropyl-1H-pyrrolo[2,3-b] pyridin-5-yl)pyridin-2-yl)-3-oxopiperazin-1-yl) propyl)piperazine-1-carboxylate: Following General step G2, the product was obtained as yellow oil (500 mg, 82%). Mass (m/z): 594.3 [M+H]⁺.

Step 2. Preparation of 1-(6-(4-chloro-3-cyclopropyl-1H-pyrrolo[2,3-b]pyridin-5-yl) pyridin-2-yl)-4-(3-(piperazin-1-yl) propyl)piperazin-2-one: Following General step B2, the product 1-(6-(4-chloro-3-cyclopropyl-1H-pyrrolo[2,3-b]pyridin-5-yl)pyridin-2-yl)-4-(3-(piperazin-1-yl) propyl)piperazin-2-one was obtained as yellow oil (600 mg, purity: 60%). Mass (m/z): 494.0 [M+H]⁺.

Step 3. Preparation of 5-(4-(3-(4-(6-(4-chloro-3-cyclopropyl-1H-pyrrolo[2,3-b]pyridin-5-yl)pyridin-2-yl)-3-oxopiperazin-1-yl) propyl)piperazin-1-yl)-2-(2,6-dioxopiperidin-3-yl)-6-fluoroisoindoline-1,3-dione: Following General step L, the product 5-(4-(3-(4-(6-(4-chloro-3-cyclopropyl-1H-pyrrolo[2,3-b]pyridin-5-yl)pyridin-2-yl)-3-oxopiperazin-1-yl) propyl)piperazin-1-yl)-2-(2,6-dioxo piperidin-3-yl)-6-fluoroisoindoline-1,3-dione was obtained as a yellow solid (90 mg, 15%). Mass (m/z): 767.8 [M+H]⁺. ¹H NMR (400 MHZ, DMSO-d₆) δ 11.85 (s, 1H), 11.14 (s, 1H), 8.33 (s, 1H), 7.96 (t, J=9.6 Hz, 2H), 7.84 (d, J=11.0 Hz, 1H), 7.63 (d, J=7.4 Hz, 2H), 7.35 (s, 1H), 5.13 (dd, J=12.8, 5.4 Hz, 1H), 4.16 (s, 4H), 3.50 (d, J=35.4 Hz, 10H), 3.25 (s, 3H), 2.88 (d, J=11.6 Hz, 2H), 2.70-2.56 (m, 2H), 2.22 (s, 1H), 2.08 (s, 3H), 0.86 (dt, J=5.7, 3.8 Hz, 2H), 0.66 (q, J=5.8 Hz, 2H).

### Example 54: 3-(5-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)-1-oxoisoindolin-2-yl)piperidine-2,6-dione

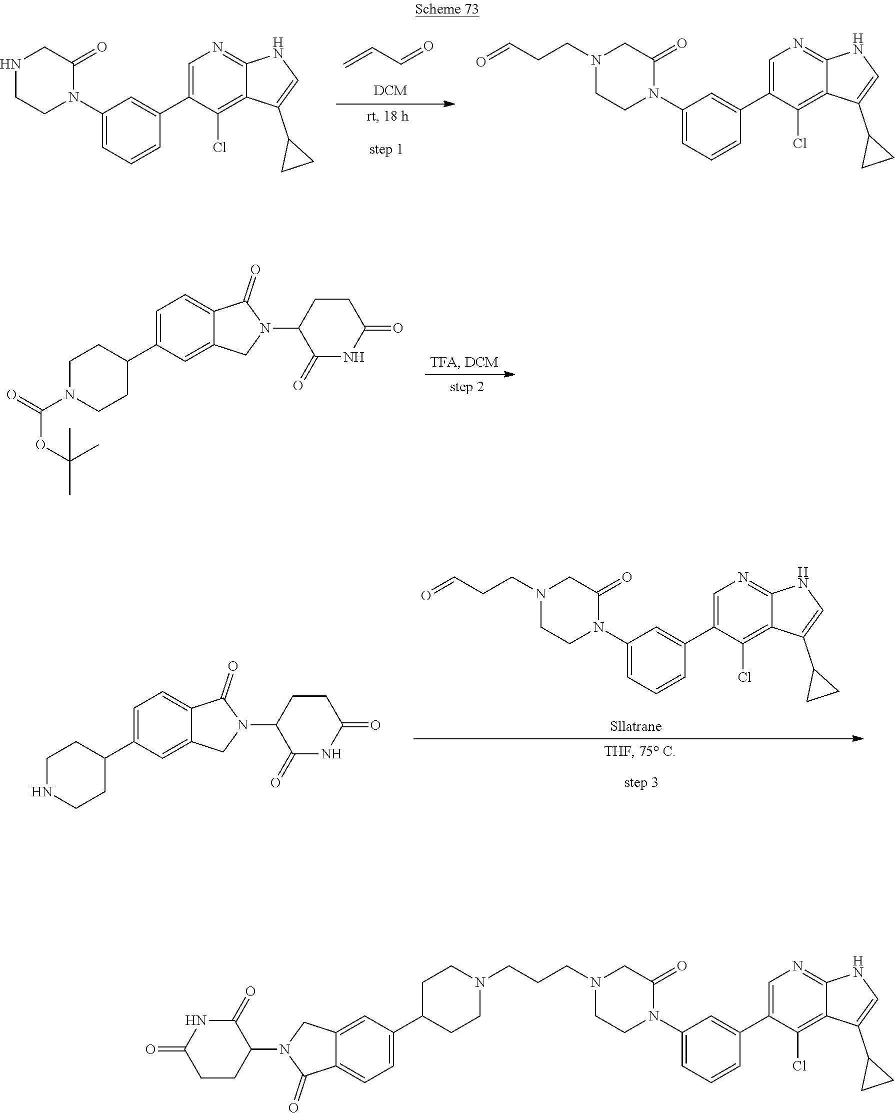

Step 1. 3-(4-(3-(4-chloro-3-cyclopropyl-1H-pyrrolo[2,3-b]pyridin-5-yl)phenyl)-3-oxopiperazin-1-yl) propanal: To a solution of 1-(3-{4-chloro-3-cyclopropyl-1H-pyrrolo[2,3-b] pyridin-3-yl}phenyl)piperazin-2-one (1000 mg, 2.7260 mmol) in DCM (10 mL) was added prop-2-enal (916.97 mg, 16.356 mmol). The reaction solution was stirred at rt for 18 hrs. concentrated under reduced pressure to give the target product (1 g, 78.06%) as a yellow solid. Mass (m/z): 423 [M+H]⁺.

Step 2. Preparation of 3-(1-oxo-5-(piperidin-4-yl) isoindolin-2-yl)piperidine-2,6-dione: Following General step B2, product 3-(1-oxo-5-(piperidin-4-yl) isoindolin-2-yl) piperidine-2,6-dione (150 mg, yield: 93.27%) was obtained as a brown solid. Mass (m/z): 328 [M+H]⁺.

Step 3. (General Step M2) Preparation of 3-(5-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)-1-oxoisoindolin-2-yl)piperidine-2,6-dione: A mixture solution of 3-[1-oxo-5-(piperidin-4-yl)-3H-isoindol-2-yl]piperidine-2,6-dione (120 mg, 0.3665 mmol), 3-[4-(3-{4-chloro-3-cyclopropyl-1H-pyrrolo[2,3-b]pyridin-3-yl}phenyl)-3-oxopiperazin-1-yl]propanal (155 mg, 0.3665 mmol) and SILATRANE (192.7 mg, 1.0995 mmol) in THF (2 mL), AcOH (two drops) was stirred at 75° C. under N₂ for 18 hrs. the mixture was concentrated under reduced pressure. The residue was purified by column chromatography (DCM/MeOH=10:1) to give crude product, further purified by prep-HPLC (Gemini-C₁₈ 150×21.2 mm, 5 um; ACN-H2O (0.1% FA)₁₀₋₃₀) to give the desired product (2.5 mg, yield: 0.87%) as a white solid. Mass (m/z): 734 [M+H]⁺. ¹H NMR (400 MHz, DMSO-d₆) δ 8.48 (s, 1H), 8.12 (s, 1H), 7.79 (d, J=7.9 Hz, 1H), 7.57 (dd, J=17.3, 9.7 Hz, 2H), 7.51-7.45 (m, 2H), 7.39 (d, J=8.3 Hz, 1H), 7.19 (s, 1H), 5.17 (dd, J=13.3, 5.2 Hz, 1H), 4.49 (q, J=17.3 Hz, 2H), 3.95-3.81 (m, 2H), 3.70 (d, J=12.0 Hz, 2H), 3.39 (s, 2H), 3.29-3.19 (m, 2H), 3.17-3.02 (m, 3H), 3.02-2.87 (m, 3H), 2.84-2.76 (m, 1H), 2.69 (t, J=6.5 Hz, 2H), 2.50 (dd, J=13.3, 4.6 Hz, 1H), 2.34-2.12 (m, 4H), 2.04 (d, J=10.4 Hz, 4H), 1.00-0.87 (m, 2H), 0.72-0.57 (m, 2H).

### Example 55: 3-(2,2-difluoroethyl)-1H-pyrrolo 3-(5-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)-1-oxoisoindolin-2-yl)piperidine-2,6-dione

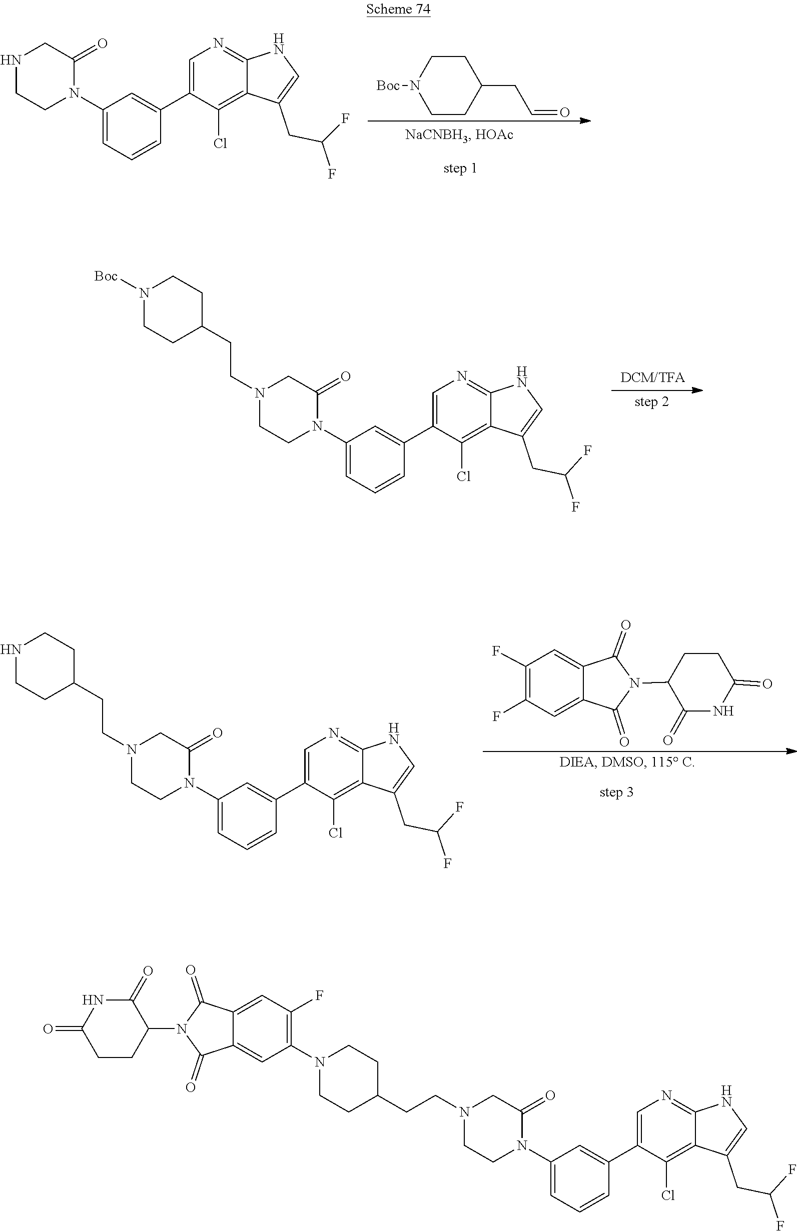 [SMILES: O=C1CNCCN1c1cccc(-c2cnc3[nH]cc(CC(F)F)c3c2Cl)c1 ; CC(C)(C)OC(=O)N1CCC(CC=O)CC1 ; CC(C)(C)OC(=O)N1CCC(CCN2CCN(c3cccc(-c4cnc5[nH]cc(CC(F)F)c5c4Cl)c3)C(=O)C2)CC1 ; O=C1CN(CCC2CCNCC2)CCN1c1cccc(-c2cnc3[nH]cc(CC(F)F)c3c2Cl)c1 ; O=C1CCC(N2C(=O)c3cc(F)c(F)cc3C2=O)C(=O)N1 ; O=C1CCC(N2C(=O)c3cc(F)c(N4CCC(CCN5CCN(c6cccc(-c7cnc8[nH]cc(CC(F)F)c8c7Cl)c6)C(=O)C5)CC4)cc3C2=O)C(=O)N1]

Step 1. Preparation of tert-butyl {4-[2-(4-{3-[4-chloro-3-(2,2-difluoroethyl)-1H-pyrrolo[2,3-b]pyridin-3-yl]phenyl}-3-oxopiperazin-1-yl)ethyl]piperidin-1-yl}formate: Following General step M1, the product was obtained as a brown solid (130 mg, 42.13%). Mass (m/z): 603.3 [M+H]⁺.

Step 2. Preparation of 1-{3-[4-chloro-3-(2,2-difluoroethyl)-1H-pyrrolo[2,3-b]pyridin-3-yl]phenyl}-4-[2-(piperidin-4-yl)ethyl]piperazin-2-one: Following General step B2, the product was obtained (70 mg, 80.76%) as pale yellow solid. Mass (m/z): 502.9 [M+H]⁺.

Step 3. Preparation of 5-{4-[2-(4-{3-[4-chloro-3-(2,2-difluoroethyl)-1H-pyrrolo[2,3-b]pyridin-3-yl]phenyl}-3-oxopiperazin-1-yl)ethyl]piperidin-1-yl}-2-(2,6-dioxopiperidin-3-yl)-6-fluoroisoindole-1,3-dione: Following General step L, the product (5 mg, 4.4%) was obtained as a yellow solid. Mass (m/z): 775.7 [M+H]⁺. ¹HNMR (400 MHZ, DMSO-d₆) δ 12.12 (d, J=2.5 Hz, 1H), 11.09 (s, 1H), 8.15 (s, 1H), 7.70 (d, J=11.4 Hz, 1H), 7.58 (d, J=2.5 Hz, 1H), 7.54 (d, J=7.8 Hz, 1H), 7.47 (t, J=1.8 Hz, 1H), 7.44 (s, 1H), 7.42 (s, 1H), 7.40 (m, 1H), 6.40-6.12 (m, 1H), 5.07 (dd, J=12.8, 5.4 Hz, 1H), 3.99 (t, J=20.0 Hz, 4H), 3.29 (s, 4H), 2.85 (m, 4H), 2.54 (m, 2H), 2.00 (m, 2H), 1.79 (d, J=11.8 Hz, 2H), 1.69 (d, J=7.1 Hz, 2H), 1.55 (s, 1H), 1.26 (m, 4H).

### Example 56: 3-(5-(1-(3-(4-(3-(4-chloro-3-cyclopropyl-1H-pyrrolo[2,3-b]pyridin-5-yl)phenyl)-3-oxopiperazin-1-yl) propyl)-1,2,3,6-tetrahydropyridin-4-yl)-1-oxoisoindolin-2-yl)piperidine-2,6-dione

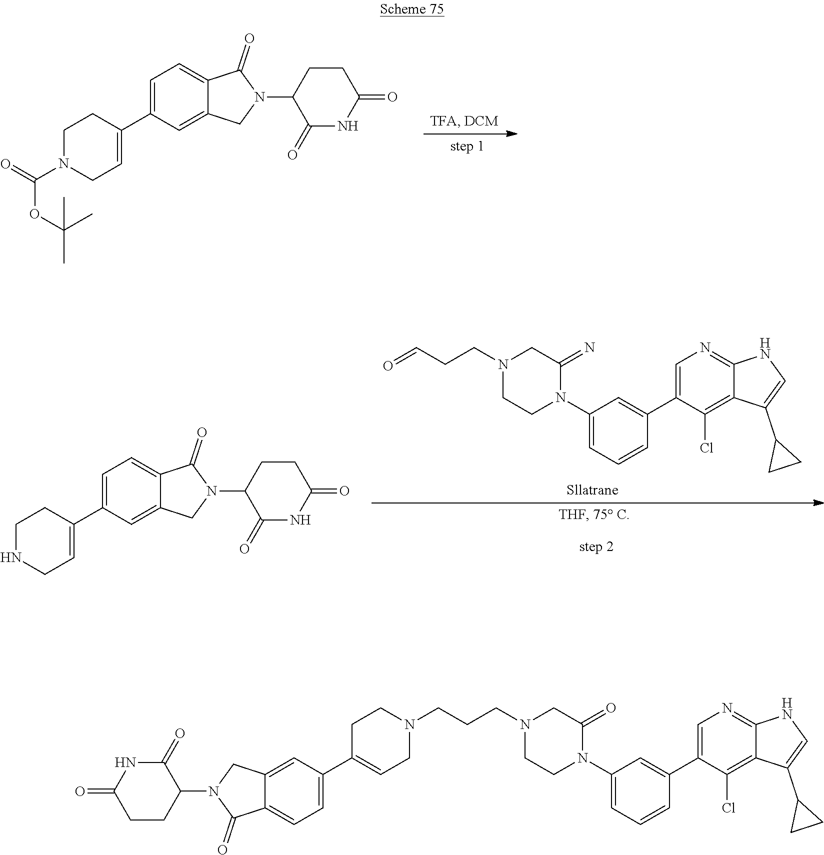 [SMILES: CC(C)(C)OC(=O)N1CC=C(c2ccc3c(c2)CN(C2CCC(=O)NC2=O)C3=O)CC1 ; O=C1CCC(N2Cc3cc(C4=CCNCC4)ccc3C2=O)C(=O)N1 ; N=C1CN(CCC=O)CCN1c1cccc(-c2cnc3[nH]cc(C4CC4)c3c2Cl)c1 ; O=C1CCC(N2Cc3cc(C4=CCN(CCCN5CCN(c6cccc(-c7cnc8[nH]cc(C9CC9)c8c7Cl)c6)C(=O)C5)CC4)ccc3C2=O)C(=O)N1]

Step 1. Preparation of 3-(1-oxo-5-(1,2,3,6-tetrahydropyridin-4-yl) isoindolin-2-yl) piperidine-2,6-dione: Following General step B2, the desired product (1 g, yield: 96.67%) was obtained as a brown solid. Mass (m/z): 326 [M+H]⁺.

Step 2. Preparation of 3-(5-(1-(3-(4-(3-(4-chloro-3-cyclopropyl-1H-pyrrolo[2,3-b]pyridin-5-yl)phenyl)-3-oxopiperazin-1-yl) propyl)-1,2,3,6-tetrahydropyridin-4-yl)-1-oxoisoindolin-2-yl)piperidine-2,6-dione: Following General step M2, the desired product (400 mg, yield: 6%) was obtained as a white solid. Mass (m/z): 732 [M+H]⁺. ¹H NMR (400 MHZ, DMSO-d₆) δ 11.75 (d, J=2.4 Hz, 1H), 10.98 (s, 1H), 8.14-8.12 (m, 1H), 7.75-7.65 (m, 2H), 7.61 (d, J=7.9 Hz, 1H), 7.55-7.45 (m, 2H), 7.40 (dd, J=11.4, 4.8 Hz, 2H), 7.31 (d, J=1.7 Hz, 1H), 6.34 (s, 1H), 5.11 (dd, J=13.3, 5.1 Hz, 1H), 4.45 (d, J=17.3 Hz, 1H), 4.32 (d, J=17.3 Hz, 1H), 3.79-3.69 (m, 2H), 3.36 (s, 3H), 3.22 (s, 3H), 2.97-2.76 (m, 5H), 2.62 (s, 5H), 2.44-2.30 (m, 1H), 2.21 (s, 1H), 2.06-1.94 (m, 1H), 1.77 (s, 2H), 0.90-0.79 (m, 2H), 0.69-0.60 (m, 2H).

### Example 57: 3-(5-(4-(2-(4-(3-(4-chloro-3-(pyridin-2-ylethynyl)-1H-pyrrolo[2,3-b]pyridin-5-yl)phenyl)-3-oxopiperazin-1-yl)-2-oxoethoxy)piperidin-1-yl)-1-oxoisoindolin-2-yl)piperidine-2,6-dione

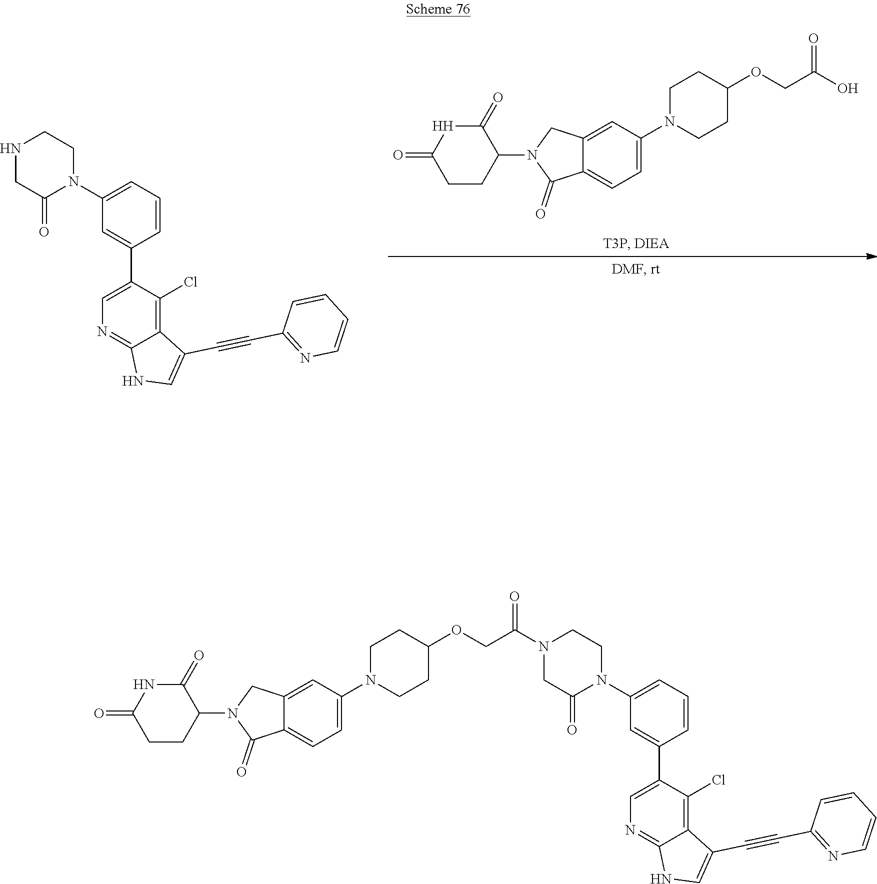

Step 1. Preparation of 3-(5-(4-(2-(4-(3-(4-chloro-3-(pyridin-2-ylethynyl)-1H-pyrrolo[2,3-b]pyridin-5-yl)phenyl)-3-oxopiperazin-1-yl)-2-oxoethoxy)piperidin-1-yl)-1-oxoisoindolin-2-yl)piperidine-2,6-dione: Following General step F, the target product (6 mg, 9.77%) was obtained as a yellow solid. Mass (m/z): 810.9 [M+H]⁺. ¹H NMR (400 MHZ, DMSO-d₆) δ 12.67 (s, 1H), 10.91 (s, 1H), 8.54 (d, J=4.5 Hz, 1H), 8.26 (s, 1H), 8.16 (s, 1H), 7.77 (td, J=7.8, 1.6 Hz, 1H), 7.50 (dd, J=7.8, 5.4 Hz, 3H), 7.48-7.37 (m, 3H), 7.31 (dd, J=7.0, 5.2 Hz, 1H), 7.02 (d, J=10.0 Hz, 2H), 5.00 (dd, J=13.3, 5.0 Hz, 1H), 4.27 (dd, J=11.4, 4.3 Hz, 4H), 4.15 (d, J=16.6 Hz, 2H), 3.81 (d, J=22.6 Hz, 4H), 3.63 (d, J=13.4 Hz, 3H), 3.04 (t, J=10.7 Hz, 2H), 2.94-2.79 (m, 1H), 2.54 (d, J=16.1 Hz, 1H), 2.39-2.25 (m, 1H), 1.92 (d, J=5.1 Hz, 3H), 1.59-1.45 (m, 2H).

### Example 58: 1-(6-(1-(3-(4-(3-(4-chloro-3-ethyl-1H-pyrrolo[2,3-b]pyridin-5-yl)phenyl)-3-oxopiperazin-1-yl) propyl)piperidin-4-yl) naphthalen-1-yl)dihydropyrimidine-2,4 (1H,3H)-dione

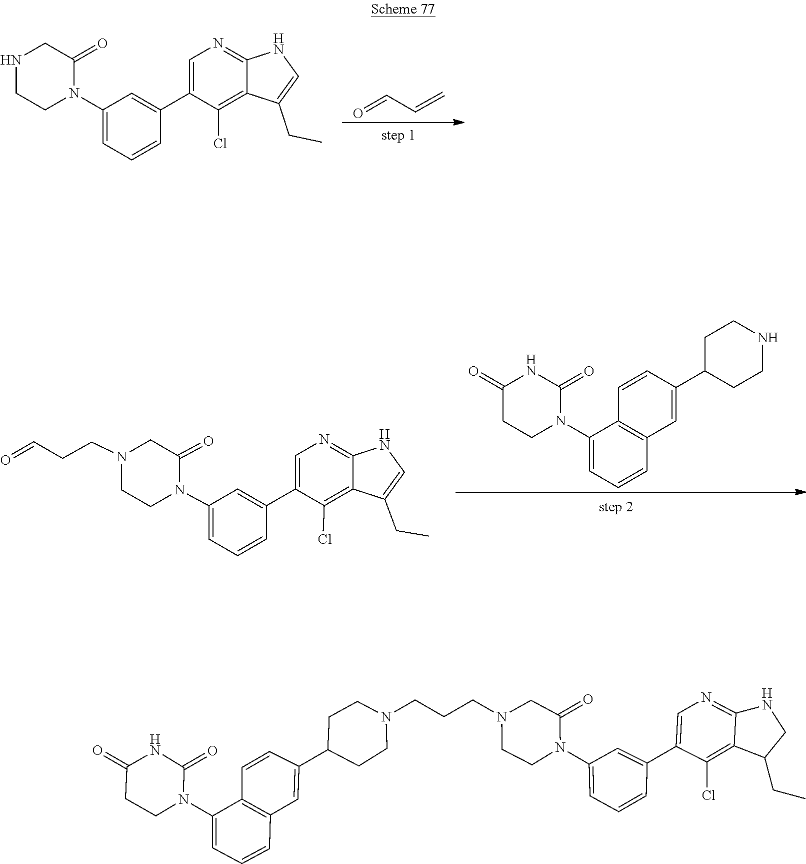

Step 1. Preparation of 3-(4-(3-(4-chloro-3-ethyl-1H-pyrrolo[2,3-b]pyridin-5-yl)phenyl)-3-oxopiperazin-1-yl) propanal: To a solution of 1-(3-{4-chloro-3-ethyl-1H-pyrrolo[2,3-b] pyridin-3-yl}phenyl)piperazin-2-one (100 mg, 0.2818 mmol) in DCM (1 mL) was added prop-2-enal (94.79 mg, 1.6908 mmol). The reaction solution was stirred at rt for 18 hrs, then concentrated under reduced pressure to give 3-(4-(3-(4-chloro-3-ethyl-1H-pyrrolo[2,3-b]pyridin-5-yl)phenyl)-3-oxopiperazin-1-yl) propanal (100 mg, 77.71%) as a yellow solid. Mass (m/z): 411 [M+H]⁺.

Step 2. Preparation of 1-(6-(1-(3-(4-(3-(4-chloro-3-ethyl-1H-pyrrolo[2,3-b]pyridin-5-yl)phenyl)-3-oxopiperazin-1-yl) propyl)piperidin-4-yl) naphthalen-1-yl)dihydropyrimidine-2,4 (1H,3H)-dione: Following General step M2, the product (9 mg, yield: 8.1%) was obtained as a white solid. Mass (m/z): 718 [M+H]⁺. ¹H NMR (400 MHZ, DMSO-d₆) δ 8.47 (s, 1H), 8.07 (d, J=3.5 Hz, 1H), 7.99-7.84 (m, 3H), 7.82-7.72 (m, 1H), 7.58-7.38 (m, 6H), 7.32 (dd, J=20.9, 8.0 Hz, 1H), 7.24 (s, 1H), 3.94 (m, 1H), 3.87-3.70 (m, 4H), 3.65 (d, J=12.1 Hz, 1H), 3.35 (s, 2H), 3.23-3.07 (m, 3H), 3.07-2.79 (m, 9H), 2.64 (t, J=6.4 Hz, 2H), 2.26-1.82 (m, 5H), 1.31 (t, J=7.4 Hz, 3H).

### Example 59: 3-(2-hydroxy-5-methylphenyl)-5-(tetrahydro-2H-pyran-4-yl)-4-(4-(trifluoro methyl)phenyl)-4,5-dihydropyrrolo[3,4-c]pyrazol-6 (2H)-one

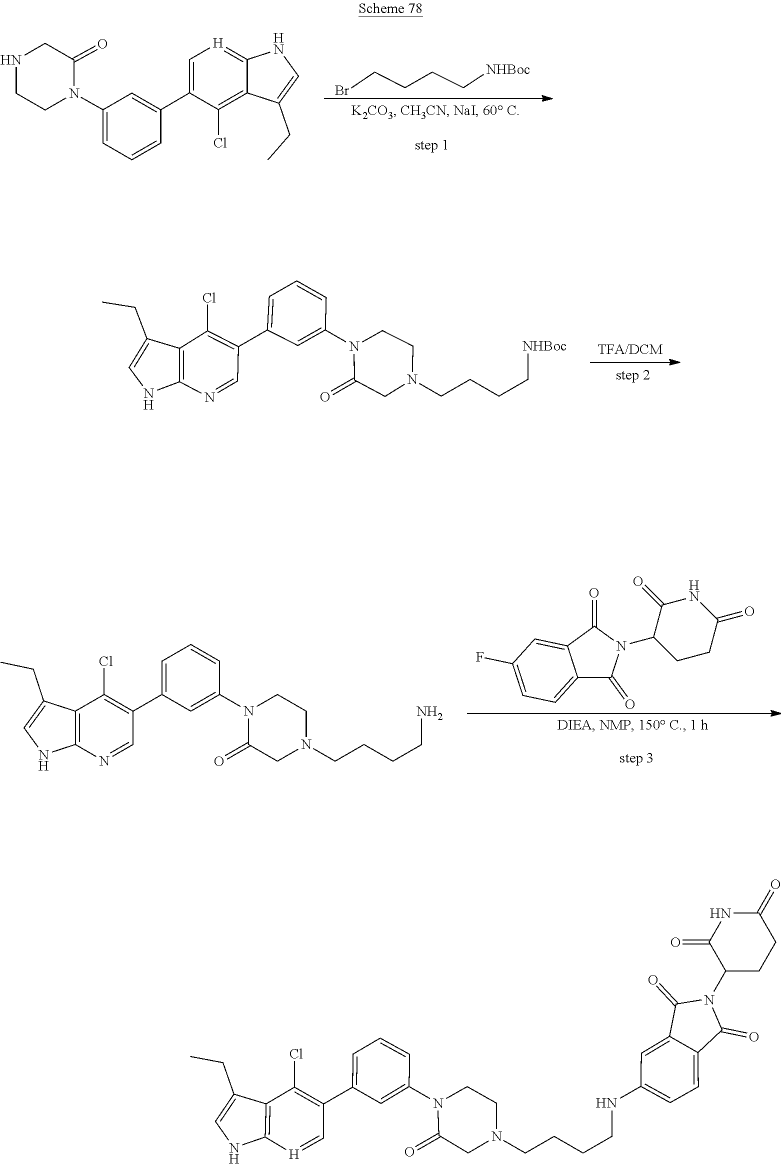

Step 1. Preparation of tert-butyl N-{4-[4-(3-{4-chloro-3-ethyl-1H-pyrrolo[2,3-b]pyridin-3-yl}phenyl)-3-oxopiperazin-1-yl]butyl}carbamate: To a mixture of 1-(3-{4-chloro-3-ethyl-1H-pyrrolo[2,3-b]pyridin-3-yl}phenyl)piperazin-2-one (300 mg, 0.846 mmol) in MeCN (10 mL) was added K₂CO₃ (234 mg, 1.69 mmol), NaI (127 mg, 0.846 mmol) and tert-butyl N-(4-bromobutyl) carbamate (320 mg, 1.29 mmol). The reaction mixture was stirred at 60° C. under N₂ for 16 hrs. The reaction mixture was concentrated under reduced pressure. The residue was diluted with water (20 mL), then extracted with EA (20 mL×3). The EA layer was washed with brine (20 mL), dried over Na₂SO₄ and concentrated under reduced pressure. The residue was purified by flash column (PE/EA=0˜40%) to give the product Tert-butyl N-{4-[4-(3-{4-chloro-3-ethyl-1H-pyrrolo[2,3-b]pyridin-3-yl}phenyl)-3-oxopiperazin-1-yl]butyl}carbamate (380 mg, 77%) as a yellow solid. Mass (m/z): 526.2 [M+H]⁺.

Step 2. Preparation of 4-(4-aminobutyl)-1-(3-{4-chloro-3-ethyl-1H-pyrrolo[2,3-b]pyridin-3-yl}phenyl)piperazin-2-one: From tert-butyl N-{4-[4-(3-{4-chloro-3-ethyl-1H-pyrrolo[2,3-b]pyridin-3-yl}phenyl)-3-oxopiperazin-1-yl]butyl}carbamate (380 mg, 0.722 mmol), following General step B2, the product 4-(4-aminobutyl)-1-(3-{4-chloro-3-ethyl-1H-pyrrolo[2,3-b]pyridin-3-yl}phenyl)piperazin-2-one (380 mg, 99%) was obtained as a yellow solid. Mass (m/z): 425.9 [M+H]⁺.

Step 3. Preparation of 5-({4-[4-(3-{4-chloro-3-ethyl-1H-pyrrolo[2,3-b]pyridin-3-yl}phenyl)-3-oxopiperazin-1-yl]butyl}amino)-2-(2,6-dioxopiperidin-3-yl) isoindole-1,3-dione: From 4-(4-aminobutyl)-1-(3-{4-chloro-3-ethyl-1H-pyrrolo[2,3-b]pyridin-3-yl}phenyl)piperazin-2-one (330 mg, 0.774 mmol), following General step L, the product 5-({4-[4-(3-{4-chloro-3-ethyl-1H-pyrrolo[2,3-b]pyridin-3-yl}phenyl)-3-oxopiperazin-1-yl]butyl}amino)-2-(2,6-dioxopiperidin-3-yl) isoindole-1,3-dione (70.0 mg, 12%) was obtained as a yellow solid. Mass (m/z): 681.8 [M+H]⁺. ¹H NMR (400 MHZ, DMSO-d₆) δ 11.83 (d, J=2.4 Hz, 1H), 11.07 (s, 1H), 8.13 (s, 1H), 7.60-7.55 (m, 2H), 7.49-7.45 (m, 2H), 7.42-7.40 (m, 2H), 6.98 (d, J=1.8 Hz, 1H), 6.88 (dd, J=8.4, 2.0 Hz, 1H), 5.04 (dd, J=12.8, 5.4 Hz, 1H), 4.10-4.00 (m, 4H), 3.69 (s, 2H), 3.27 (dt, J=13.4, 7.2 Hz, 4H), 2.95-2.83 (m, 3H), 2.60-2.52 (m, 2H), 2.46 (d, J=4.4 Hz, 1H), 2.01-1.98 (m, 1H), 1.81 (d, J=7.4 Hz, 2H), 1.66-1.63 (m, 2H), 1.27 (t, J=7.4 Hz, 3H).

### Example 60: 5-((2-(4-(3-(4-chloro-3-ethyl-1H-pyrrolo[2,3-b]pyridin-5-yl)phenyl)-3-oxo piperazin-1-yl)ethyl)amino)-2-(2,6-dioxopiperidin-3-yl)isoindoline-1,3-dione

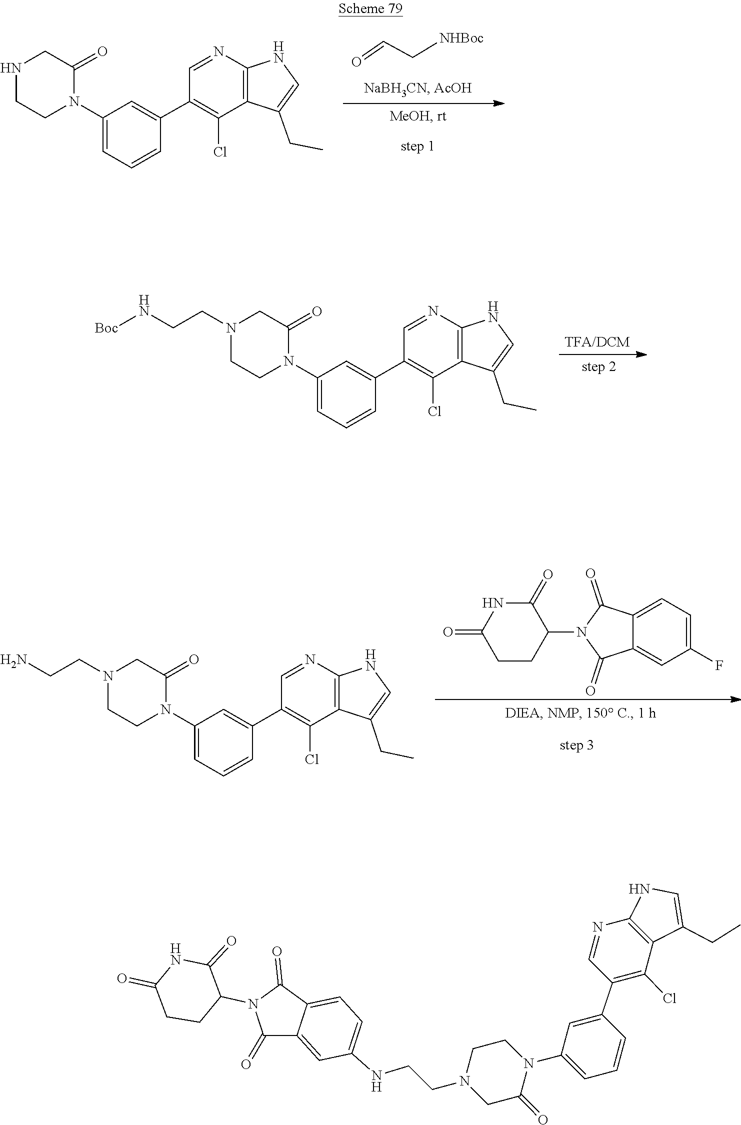 [SMILES: CCc1c[nH]c2ncc(-c3cccc(N4CCNCC4=O)c3)c(Cl)c12 ; CC(C)(C)OC(=O)NCC=O ; CCc1c[nH]c2ncc(-c3cccc(N4CCN(CCNC(=O)OC(C)(C)C)CC4=O)c3)c(Cl)c12 ; CCc1c[nH]c2ncc(-c3cccc(N4CCN(CCN)CC4=O)c3)c(Cl)c12 ; O=C1CCC(N2C(=O)c3ccc(F)cc3C2=O)C(=O)N1 ; CCc1c[nH]c2ncc(-c3cccc(N4CCN(CCNc5ccc6c(c5)C(=O)N(C5CCC(=O)NC5=O)C6=O)CC4=O)c3)c(Cl)c12]

Step 1. Preparation of tert-butyl (2-(4-(3-(4-chloro-3-ethyl-1H-pyrrolo[2,3-b]pyridin-5-yl)phenyl)-3-oxopiperazin-1-yl)ethyl) carbamate: Following General step M1, the product was obtained as a white solid (550 mg, 83%). Mass (m/z): 498.1 [M+H]⁺.

Step 2. Preparation of 4-(2-aminoethyl)-1-(3-(4-chloro-3-ethyl-1H-pyrrolo[2,3-b]pyridin-5-yl)phenyl)piperazin-2-one: Following General step B2, the product was obtained as yellow oil (1.2 g, purity: 60%). Mass (m/z): 398.1 [M+H]⁺.

Step 3. Preparation of 5-((2-(4-(3-(4-chloro-3-ethyl-1H-pyrrolo[2,3-b]pyridin-5-yl)phenyl)-3-oxopiperazin-1-yl)ethyl)amino)-2-(2,6-dioxopiperidin-3-yl)isoindoline-1,3-dione: Following General step L, the product 5-((2-(4-(3-(4-chloro-3-ethyl-1H-pyrrolo[2,3-b]pyridin-5-yl)phenyl)-3-oxopiperazin-1-yl)ethyl)amino)-2-(2,6-dioxopiperidin-3-yl)isoindoline-1,3-dione was obtained as a yellow solid (70 mg, 8%). Mass (m/z): 654.0 [M+H]⁺. ¹H NMR (400 MHZ, DMSO-d₆) ¿ 11.81 (s, 1H), 11.08 (s, 1H), 8.13 (s, 1H), 7.60 (d, J=8.2 Hz, 1H), 7.57-7.36 (m, 5H), 7.13 (s, 1H), 7.07 (s, 1H), 6.94 (d, J=8.4 Hz, 1H), 5.04 (dd, J=12.8, 5.4 Hz, 1H), 3.81 (s, 2H), 3.37 (s, 4H), 2.92 (dd, J=14.8, 7.6 Hz, 3H), 2.88 (s, 1H), 2.69-2.51 (m, 5H), 2.05-1.95 (m, 1H), 1.28 (t, J=7.4 Hz, 3H).

### Example 61: 3-(6-(4-(2-(4-(3-(4-chloro-3-cyclopropyl-1H-pyrrolo[2,3-b]pyridin-5-yl)phenyl)-3-oxopiperazin-1-yl)ethyl)piperidin-1-yl)-5-fluoro-1-oxoisoindolin-2-yl)piperidine-2,6-dione

### Example 62: 3-(5-(4-(2-(4-(3-(4-chloro-3-cyclopropyl-1H-pyrrolo[2,3-b]pyridin-5-yl)phenyl)-3-oxopiperazin-1-yl)ethyl)piperidin-1-yl)-6-fluoro-1-oxoisoindolin-2-yl)piperidine-2,6-dione

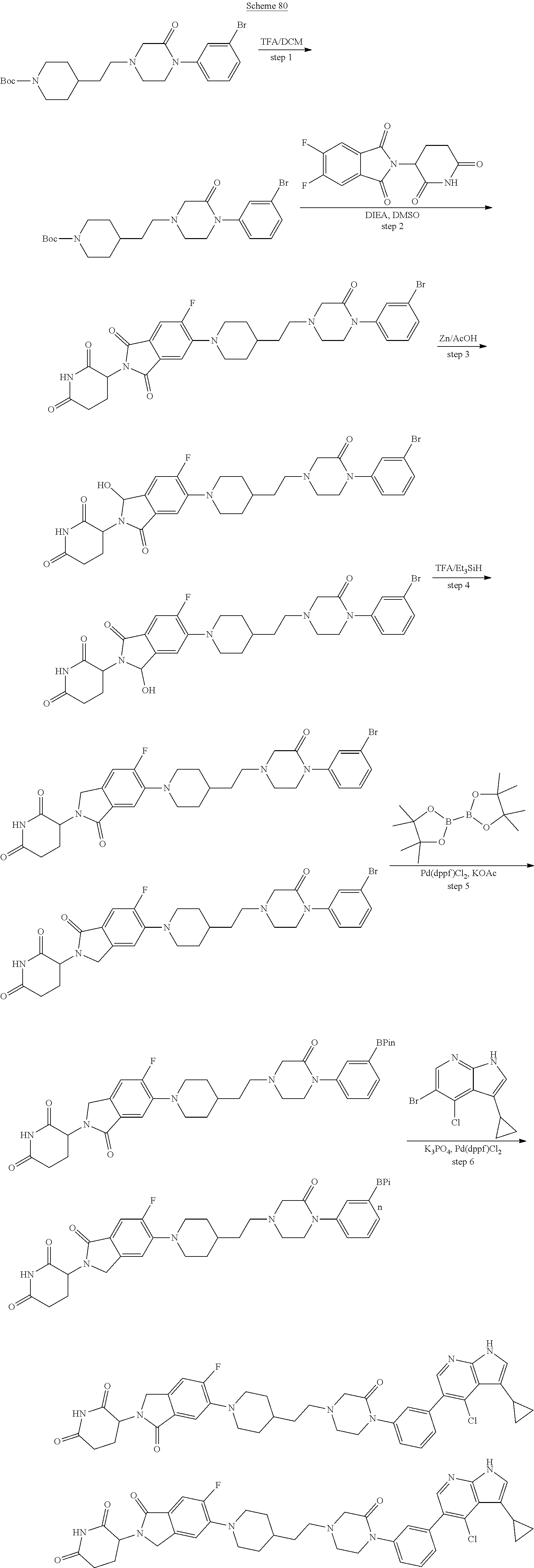

Step 1. Preparation of 1-(3-bromophenyl)-4-[2-(piperidin-4-yl)ethyl]piperazin-2-one: Following General step B2, the product (2.0 g, 95%) was obtained as a yellow solid. Mass (m/z): 366.1 [M+H]⁺.

Step 2. Preparation of 5-(4-{2-[4-(3-bromophenyl)-3-oxopiperazin-1-yl]ethyl}piperidin-1-yl)-2-(2,6-dioxopiperidin-3-yl)-6-fluoroisoindole-1,3-dione: Following General step L, the product was obtained (1.8 g, 62%) as a white solid. Mass (m/z): 340.9 [M+H]⁺.

Step 3. Preparation of 3-(5-(4-(2-(4-(3-bromophenyl)-3-oxopiperazin-1-yl)ethyl) piperidin-1-yl)-6-fluoro-3-hydroxy-1-oxoisoindolin-2-yl)piperidine-2,6-dione and 3-(5-(4-(2-(4-(3-bromophenyl)-3-oxopiperazin-1-yl)ethyl)piperidin-1-yl)-6-fluoro-1-hydroxy-3-oxoisoindolin-2-yl)piperidine-2,6-dione: To a mixture of 5-(4-{2-[4-(3-bromophenyl)-3-oxopiperazin-1-yl]ethyl}piperidin-1-yl)-2-(2,6-dioxopiperidin-3-yl)-6-fluoroisoindole-1,3-dione (1.75 g, 2.73 mmol) in HOAc (20 mL) was added Zn (1.78 g, 27.32 mmol). The reaction mixture was stirred at 90° C. for 16 hrs. The reaction mixture was filtered and the filtrate was concentrated under reduced pressure to give a mixture of desired products (2.2 g, 96%) as brown oil. Mass (m/z): 641.7 [M+H].

Step 4. Preparation of 3-(5-(4-(2-(4-(3-bromophenyl)-3-oxopiperazin-1-yl)ethyl) piperidin-1-yl)-6-fluoro-1-oxoisoindolin-2-yl)piperidine-2,6-dione and 3-(6-(4-(2-(4-(3-bromophenyl)-3-oxopiperazin-1-yl)ethyl)piperidin-1-yl)-5-fluoro-1-oxoisoindolin-2-yl)piperidine-2,6-dione: To a mixture of 3-(5-(4-(2-(4-(3-bromophenyl)-3-oxopiperazin-1-yl)ethyl)piperidin-1-yl)-6-fluoro-3-hydroxy-1-oxoisoindolin-2-yl)piperidine-2,6-dione and 3-(5-(4-(2-(4-(3-bromophenyl)-3-oxopiperazin-1-yl)ethyl)piperidin-1-yl)-6-fluoro-1-hydroxy-3-oxoisoindolin-2-yl)piperidine-2,6-dione (2.2 g, 3.4 mmol) in HOAc (20 mL) was added Et₃SiH (9.33 g, 0.080 mol) and TFA (26.8 g, 0.235 mol). The reaction mixture was stirred at 70° C. for 3 hrs. The reaction mixture was concentrated under reduced pressure. The residue was purified by flash column (DCM/MeOH=0˜4%) to give a mixture of desired products (1.2 g, 50%) as a white solid. Mass (m/z): 625.8 [M+H]⁺.

Step 5. Preparation of 3-(5-fluoro-1-oxo-6-(4-(2-(3-oxo-4-(3-(4,4,5,5-tetramethyl-1,3,2-dioxaborolan-2-yl)phenyl)piperazin-1-yl)ethyl)piperidin-1-yl) isoindolin-2-yl)piperidine-2,6-dione and 3-(6-fluoro-1-oxo-5-(4-(2-(3-oxo-4-(3-(4,4,5,5-tetramethyl-1,3,2-dioxaborolan-2-yl)phenyl)piperazin-1-yl)ethyl)piperidin-1-yl) isoindolin-2-yl)piperidine-2,6-dione: To a mixture of 3-(5-(4-(2-(4-(3-bromophenyl)-3-oxopiperazin-1-yl)ethyl)piperidin-1-yl)-6-fluoro-1-oxoisoindolin-2-yl)piperidine-2,6-dione and 3-(6-(4-(2-(4-(3-bromophenyl)-3-oxopiperazin-1-yl)ethyl)piperidin-1-yl)-5-fluoro-1-oxoisoindolin-2-yl)piperidine-2,6-dione (1.2 g, 1.9 mmol) in dioxane (20 mL) was added KOAc (0.56 g, 5.7 mmol), 4,4,5,5-tetramethyl-2-(4,4,5,5-tetramethyl-1,3,2-dioxaborolan-2-yl)-1,3,2-dioxaborolane (580 mg, 2.2 mmol) and Pd(dppf)Cl₂ (140 mg, 0.02 mmol). The reaction mixture was stirred at 110° C. under N₂ for 2 hrs. The reaction mixture was filtered and the filtrate was concentrated under reduced pressure. The residue was diluted with water (30 mL), then extracted with EA (30 mL×3), washed with brine (50 mL), dried over Na₂SO₄ and concentrated under reduced pressure. The residue was purified by flash column (DCM/MeOH=0˜4%) to give a mixture of desired products (700 mg, 37%) as a yellow solid. Mass (m/z): 673.9 [M+H]⁺.

Step 6. Preparation of 3-(6-(4-(2-(4-(3-(4-chloro-3-cyclopropyl-1H-pyrrolo[2,3-b]pyridin-5-yl)phenyl)-3-oxopiperazin-1-yl)ethyl)piperidin-1-yl)-5-fluoro-1-oxoiso indolin-2-yl)piperidine-2,6-dione and 3-(5-(4-(2-(4-(3-(4-chloro-3-cyclopropyl-1H-pyrrolo[2,3-b]pyridin-5-yl)phenyl)-3-oxopiperazin-1-yl)ethyl)piperidin-1-yl)-6-fluoro-1-oxoisoindolin-2-yl)piperidine-2,6-dione: From 3-(5-fluoro-1-oxo-6-(4-(2-(3-oxo-4-(3-(4,4,5,5-tetramethyl-1,3,2-dioxaborolan-2-yl)phenyl)piperazin-1-yl)ethyl)piperidin-1-yl) isoindolin-2-yl)piperidine-2,6-dione and 3-(6-fluoro-1-oxo-5-(4-(2-(3-oxo-4-(3-(4,4,5,5-tetramethyl-1,3,2-dioxaborolan-2-yl)phenyl)piperazin-1-yl)ethyl)piperidin-1-yl) isoindolin-2-yl)piperidine-2,6-dione (250 mg, 0.371 mmol), following General step A, a mixture of two desired products (85 mg, 15%) was obtained as a white solid. Mass (m/z): 737.8 [M+H]⁺. The mixture (85 mg, 0.115 mmol) was separated by SFC [column: chiralpak-OJ; 250 mm×20 mm, 5 um; mobile phase: CO₂-EtOH] to give:

3-(6-(4-(2-(4-(3-(4-chloro-3-cyclopropyl-1H-pyrrolo[2,3-b]pyridine-5-yl)phenyl)-3-oxopiperazin-1-yl)ethyl)piperidin-1-yl)-5-fluoro-1-oxoiso indolin-2-yl)piperidine-2,6-dione: (8 mg, white solid, RT=5.38 min), Mass (m/z): 737.8 [M+H]⁺. ¹H NMR (400 MHZ, DMSO-d₆) δ 11.78 (d, J=2.2 Hz, 1H), 10.99 (s, 1H), 8.13 (s, 1H), 7.49 (s, 2H), 7.44-7.41 (m, 3H), 7.33-7.29 (M, 2H), 5.08 (dd, J=13.2, 5.0 Hz, 1H), 4.36 (d, J=17.2 Hz, 1H), 4.23 (d, J=17.2 Hz, 1H), 3.75 (s, 2H), 3.20 (s, 2H), 2.95-2.86 (m, 2H), 2.72-2.66 (m, 2H), 2.61-2.57 (m, 1H), 2.40-2.32 (m, 2H), 2.24-2.17 (m, 1H), 2.02-1.95 (m, 2H), 1.82 (d, J=11.4 Hz, 2H), 1.51 (s, 2H), 1.36 (d, J=12.4 Hz, 2H), 1.23 (s, 4H), 0.86-0.84 (m, 3H), 0.65-0.64 (m, 2H).

3-(5-(4-(2-(4-(3-(4-chloro-3-cyclo propyl-1H-pyrrolo[2,3-b]pyridin-5-yl)phenyl)-3-oxopiperazin-1-yl)ethyl)piperidin-1-yl)-6-fluoro-1-oxoisoindolin-2-yl)piperidine-2,6-dione: (6 mg, white solid, RT=5.41 min), Mass (m/z): 737.8 [M+H]⁺. ¹H NMR (400 MHZ, DMSO-d₆) δ 11.79 (s, 1H), 11.00 (s, 1H), 8.13 (s, 1H), 7.70-7.73 (m, 3H), 7.67-7.65 (m, 3H), 7.31 (d, J=7.8 Hz, 1H), 2.94-2.86 (m, 1H), 2.76-2.67 (m, 2H), 2.59 (d, J=16.7 Hz, 1H), 2.40-2.33 (m, 1H), 2.22-2.14 (m, 2H), 1.98 (dd, J=10.2, 5.1 Hz, 1H), 1.82 (d, J=11.5 Hz, 2H), 1.67-1.60 (m, 7H), 1.42-1.32 (m, 9H), 0.86-0.84 (m, 3H), 0.66-0.62 (m, 2H).

### Example 63: 3-(5-(4-(3-(4-(3-(4-chloro-3-cyclopropyl-1H-pyrrolo[2,3-b]pyridin-5-yl)phenyl)-3-oxopiperazin-1-yl) propyl)piperazin-1-yl)-1-oxoisoindolin-2-yl)piperidine-2,6-dione

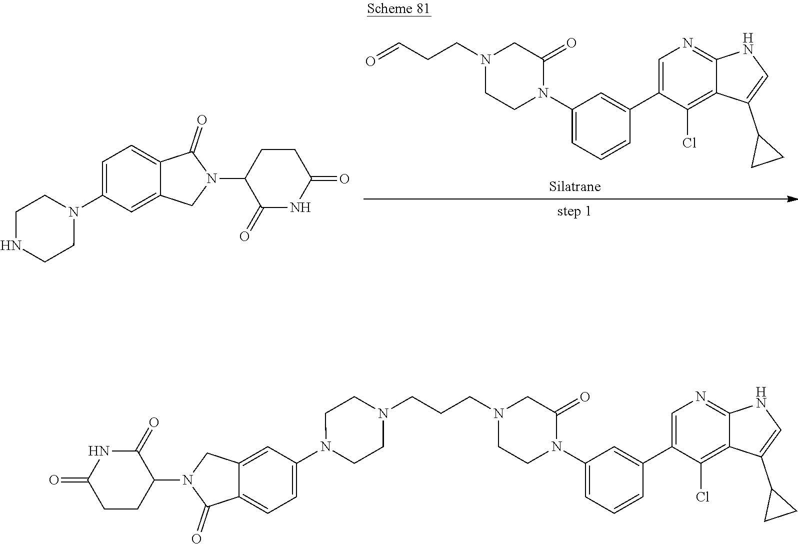 [SMILES: O=C1CCC(N2Cc3cc(N4CCNCC4)ccc3C2=O)C(=O)N1 ; O=CCCN1CCN(c2cccc(-c3cnc4[nH]cc(C5CC5)c4c3Cl)c2)C(=O)C1 ; O=C1CCC(N2Cc3cc(N4CCN(CCCN5CCN(c6cccc(-c7cnc8[nH]cc(C9CC9)c8c7Cl)c6)C(=O)C5)CC4)ccc3C2=O)C(=O)N1]

Step 1. Preparation of 3-(5-(4-(3-(4-(3-(4-chloro-3-cyclopropyl-1H-pyrrolo[2,3-b] pyridin-5-yl)phenyl)-3-oxopiperazin-1-yl) propyl)piperazin-1-yl)-1-oxoisoindolin-2-yl) piperidine-2,6-dione: Following General step M2, desired product (31 mg, yield: 13.17%) was obtained as a white solid. Mass (m/z): 735 [M+H]⁺. ¹H NMR (400 MHz, DMSO-d₆) δ 11.76 (d, J=2.2 Hz, 1H), 10.96 (s, 1H), 8.13 (d, J=5.5 Hz, 1H), 7.58 (d, J=8.0 Hz, 1H), 7.50 (dd, J=16.4, 8.6 Hz, 2H), 7.44-7.36 (m, 2H), 7.32 (d, J=1.8 Hz, 1H), 7.16 (s, 2H), 5.06 (dd, J=13.3, 5.0 Hz, 1H), 4.35 (d, J=16.8 Hz, 1H), 4.23 (d, J=17.0 Hz, 1H), 3.76 (d, J=5.4 Hz, 2H), 3.43 (d, J=80.0 Hz, 11H), 2.98-2.79 (m, 3H), 2.70-2.52 (m, 3H), 2.47-2.29 (m, 2H), 2.20 (m, 1H), 2.02-1.72 (m, 3H), 1.24 (s, 1H), 0.85 (ddd, J=8.2, 6.0, 4.0 Hz, 2H), 0.70-0.59 (m, 2H).

### Example 64: 3-(5-(8-(3-(4-(3-(4-chloro-3-cyclopropyl-1H-pyrrolo[2,3-b]pyridin-5-yl)phenyl)-3-oxopiperazin-1-yl) propyl)-8-azabicyclo[3.2.1]oct-2-en-3-yl)-1-oxoisoindolin-2-yl)piperidine-2,6-dione

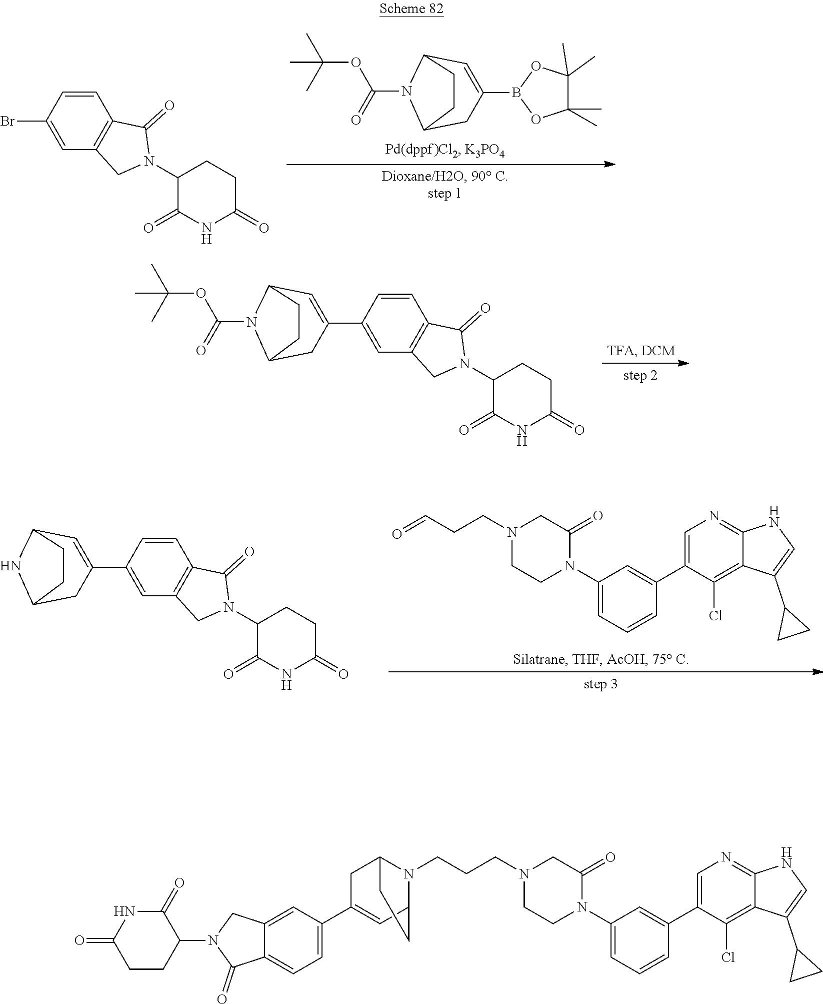

Step 1. Preparation of tert-butyl 3-(2-(2,6-dioxopiperidin-3-yl)-1-oxoisoindolin-5-yl)-8-azabicyclo[3.2.1]oct-2-ene-8-carboxylate: To a mixture of compound 1 (500 mg, 1.5473 mmol), compound 2 (622.47 g, 1.8567 mmol) and K₃PO₄ (394.13, 1.8567 mmol) in dioxane/H2O (10:1, 100 mL) under N₂ was added Pd(dppf)Cl₂ (56.61 mg, 0.0773 mmol). The reaction mixture was stirred at 90° C. for 18 hrs. 5% citric acid water (20 mL) was added and extracted with DCM (20 mL×2). The organic layer was washed with brine (20 mL×2) dried over Na₂SO₄ and concentrated, the residue was purified by combi-flash with MeOH/DCM (1:10) to afford target compound (500 mg, yield: 67.99%) as a brown solid. Mass (m/z): 452 [M+H]⁺.

Step 2. Preparation of 3-(5-(8-azabicyclo[3.2.1]oct-2-en-3-yl)-1-oxoisoindolin-2-yl) piperidine-2,6-dione: Following General step B2, the desired product (350 mg, yield: 85.44%) was obtained as a brown solid. Mass (m/z): 352 [M+H]⁺.

Step 3. Preparation of 3-(5-(8-(3-(4-(3-(4-chloro-3-cyclopropyl-1H-pyrrolo[2,3-b] pyridin-5-yl)phenyl)-3-oxopiperazin-1-yl) propyl)-8-azabicyclo[3.2.1]oct-2-en-3-yl)-1-oxoisoindolin-2-yl)piperidine-2,6-dione: Following General step M2, the desired product (32 mg, yield: 7.04%) was obtained as a white solid. Mass (m/z): 758 [M+H]⁺. ¹H NMR (400 MHZ, DMSO-d₆) δ 11.75 (d, J=2.2 Hz, 1H), 10.98 (s, 1H), 8.23 (s, 1H), 8.12 (s, 1H), 7.71-7.60 (m, 2H), 7.57 (d, J=8.1 Hz, 1H), 7.53-7.44 (m, 2H), 7.43-7.35 (m, 2H), 7.30 (d, J=1.7 Hz, 1H), 6.52 (d, J=5.4 Hz, 1H), 5.10 (dd, J=13.3, 5.1 Hz, 1H), 4.42 (d, J=17.3 Hz, 1H), 4.30 (d, J=17.4 Hz, 1H), 3.77-3.69 (m, 2H), 3.56 (s, 1H), 3.51 (s, 1H), 3.18 (s, 2H), 2.97-2.83 (m, 2H), 2.82-2.76 (m, 3H), 2.65-2.52 (m, 3H), 2.48-2.32 (m, 3H), 2.25-2.15 (m, 1H), 2.08 (d, J=16.8 Hz, 2H), 2.02-1.88 (m, 2H), 1.81 (s, 1H), 1.74-1.63 (m, 2H), 1.55 (s, 1H), 0.85 (dq, J=5.8, 4.0 Hz, 2H), 0.69-0.59 (m, 2H).

### Example 65: 3-(6-((8-(4-(3-(4-chloro-3-ethyl-1H-pyrrolo[2,3-b]pyridin-5-yl)phenyl)-3-oxopiperazin-1-yl)-8-oxooctyl)oxy)-1-oxoisoindolin-2-yl)piperidine-2,6-dione

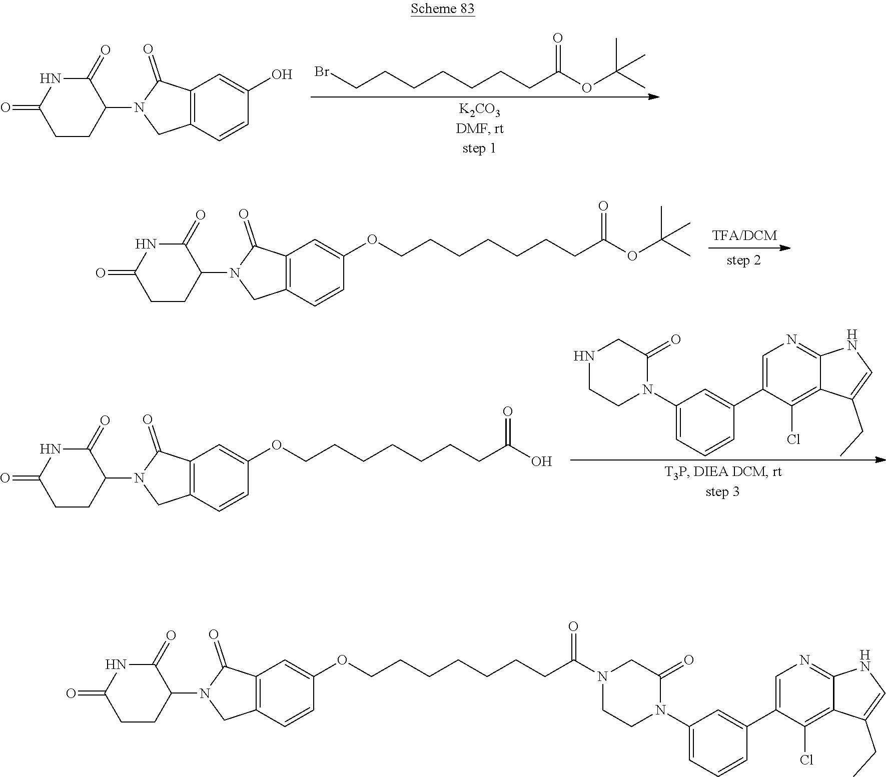 [SMILES: O=C1CCC(N2Cc3ccc(O)cc3C2=O)C(=O)N1 ; CC(C)(C)OC(=O)CCCCCCCBr ; CC(C)(C)OC(=O)CCCCCCCOc1ccc2c(c1)C(=O)N(C1CCC(=O)NC1=O)C2 ; O=C(O)CCCCCCCOc1ccc2c(c1)C(=O)N(C1CCC(=O)NC1=O)C2 ; CCc1c[nH]c2ncc(-c3cccc(N4CCNCC4=O)c3)c(Cl)c12 ; CCc1c[nH]c2ncc(-c3cccc(N4CCN(C(=O)CCCCCCCOc5ccc6c(c5)C(=O)N(C5CCC(=O)NC5=O)C6)CC4=O)c3)c(Cl)c12]

Step 1. (General Step E2) Preparation of tert-butyl 8-((2-(2,6-dioxopiperidin-3-yl)-3-oxoisoindolin-5-yl)oxy) octanoate: To a solution of 3-(6-hydroxy-1-oxo-2,3-dihydro-1H-isoindol-2-yl)piperidine-2,6-dione (0.2 g, 768 umol) in DMF (4 mL) was added dipotassium carbonate (106 mg, 768 umol) and tert-butyl 2-bromoacetate (107 mg, 384 umol). The mixture was stirred at 25° C. for 12 h. To the reaction mixture was added water (10 mL), and the aqueous phase was extracted with EtOAc (10 mL*3). The combined organic layers were dried with Na₂SO₄ and concentrated under reduced pressure. The crude was purified by silica column chromatography (PE:THF=1:1). 0.065 g of crude product was obtained. Then the crude was triturated with MTBE (5 mL). The filtrate was collected by filtration, washed with MTBE (3 mL) and dried under reduced pressure to give product (50 mg, 15.11%). Mass (m/z): 459.3 [M+H]⁺.

Step 2. Preparation of 8-((2-(2,6-dioxopiperidin-3-yl)-3-oxoisoindolin-5-yl)oxy)octanoic acid: Following General step B2, the product 8-((2-(2,6-dioxopiperidin-3-yl)-3-oxoisoindolin-5-yl)oxy) octanoic acid was obtained as yellow oil (50 mg, purity: 60%). Mass (m/z): 403.0 [M+H]⁺.

Step 3. Preparation of 3-(6-((8-(4-(3-(4-chloro-3-ethyl-1H-pyrrolo[2,3-b]pyridin-5-yl)phenyl)-3-oxopiperazin-1-yl)-8-oxooctyl)oxy)-1-oxoisoindolin-2-yl)piperidine-2,6-dione: Following General step F, the product 3-(6-((8-(4-(3-(4-chloro-3-ethyl-1H-pyrrolo[2,3-b]pyridin-5-yl)phenyl)-3-oxopiperazin-1-yl)-8-oxooctyl)oxy)-1-oxoisoindolin-2-yl)piperidine-2,6-dione was obtained as a white solid (30 mg, 40%). Mass (m/z): 738.7 [M+H]⁺. ¹H NMR (400 MHz, CD₃OD) δ 8.14 (s, 1H), 7.52 (d, J=7.7 Hz, 1H), 7.46-7.37 (m, 3H), 7.35-7.28 (m, 2H), 7.13 (d, J=2.4 Hz, 1H), 7.02 (dd, J=8.2, 2.4 Hz, 1H), 5.11-5.02 (m, 1H), 4.39-4.28 (m, 4H), 3.95-3.90 (m, 2H), 3.88-3.84 (m, 1H), 3.78 (s, 1H), 3.71 (dd, J=7.4, 5.8 Hz, 2H), 2.97 (t, J=7.4 Hz, 2H), 2.85 (dd, J=5.0, 2.4 Hz, 2H), 2.42 (dd, J=9.8, 7.2 Hz, 3H), 2.13-2.04 (m, 1H), 1.64-1.56 (m, 2H), 1.54-1.46 (m, 2H), 1.31 (t, J=7.4 Hz, 9H).

### Example 66: 3-ethyl-1H-pyrrolo 3-(6-(2-(4-(3-(4-chloro-3-ethyl-1H-pyrrolo[2,3-b]pyridin-5-yl)phenyl)-3-oxopiperazin-1-yl)-2-oxoethoxy)-1-oxoisoindolin-2-yl)piperidine-2,6-dione

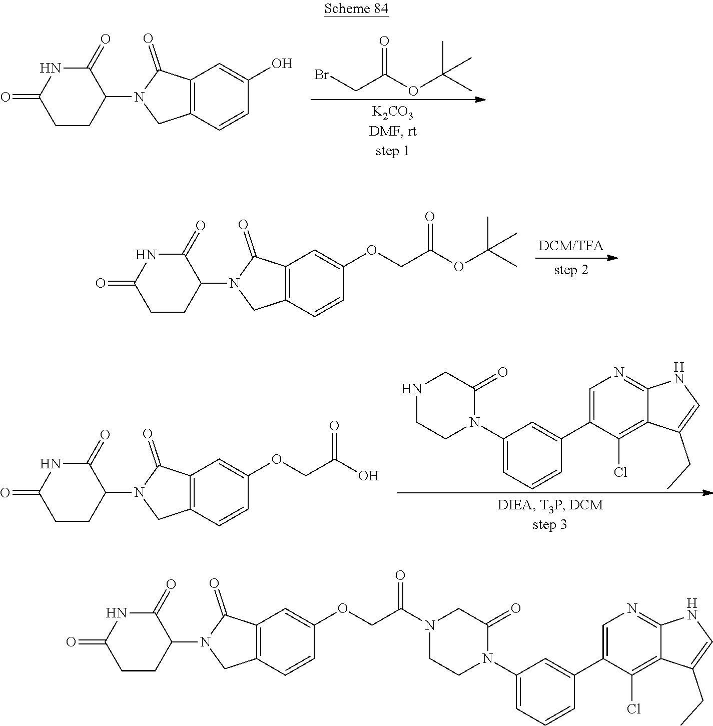

Step 1. Preparation of tert-butyl 2-((2-(2,6-dioxopiperidin-3-yl)-3-oxoisoindolin-5-yl)oxy)acetate: Following General step E2, the desired product was obtained as white solid (50 mg, 23%). Mass (m/z): 375.2 [M+H]⁺.

Step 2. Preparation of {[2-(2,6-dioxopiperidin-3-yl)-3-oxo-1H-isoindol-5-yl]oxy}acetic acid: Following General step B2, the product {[2-(2,6-dioxopiperidin-3-yl)-3-oxo-1H-isoindol-5-yl]oxy}acetic acid was obtained as colorless oil (42 mg, 94%). Mass (m/z): 318.9 [M+H]⁺.

Step 3. Preparation of 3-(6-{2-[4-(3-{4-chloro-3-ethyl-1H-pyrrolo[2,3-b]pyridin-3-yl}phenyl)-3-oxopiperazin-1-yl]-2-oxoethoxy}-1-oxo-3H-isoindol-2-yl)piperidine-2,6-dione: Following General step F, the product 3-(6-{2-[4-(3-{4-chloro-3-ethyl-1H-pyrrolo[2,3-b]pyridin-3-yl}phenyl)-3-oxopiperazin-1-yl]-2-oxoethoxy}-1-oxo-3H-isoindol-2-yl)piperidine-2,6-dione (45 mg, 52%) was obtained as a white solid. Mass (m/z): 655.0 [M+H]⁺. ¹H NMR (400 MHz, CD₃OD) δ 8.11 (s, 1H), 7.56 (t, J=7.8 Hz, 1H), 7.50-7.30 (m, 6H), 7.27 (s, 1H), 5.13 (dd, J=13.2, 5.2 Hz, 1H), 5.02 (d, J=3.0 Hz, 2H), 4.48-4.34 (m, 4H), 4.05-3.94 (m, 4H), 3.97-4.04 (m, 3H), 3.85 (t, J=8.0 Hz, 1H), 3.00 (q, J=7.4, 0.8 Hz, 3H), 2.89-2.85 (m, 1H), 2.79-2.74 (m, 1H), 2.51-2.43 (m, 1H), 2.19-2.13 (m, 1H), 1.33 (t, J=7.4 Hz, 3H).

### Example 67: 3-ethyl-1H-pyrrolo 3-(6-(4-(4-(3-(4-chloro-3-ethyl-1H-pyrrolo[2,3-b]pyridin-5-yl)phenyl)-3-oxopiperazin-1-yl)-4-oxobutoxy)-1-oxoisoindolin-2-yl)piperidine-2,6-dione

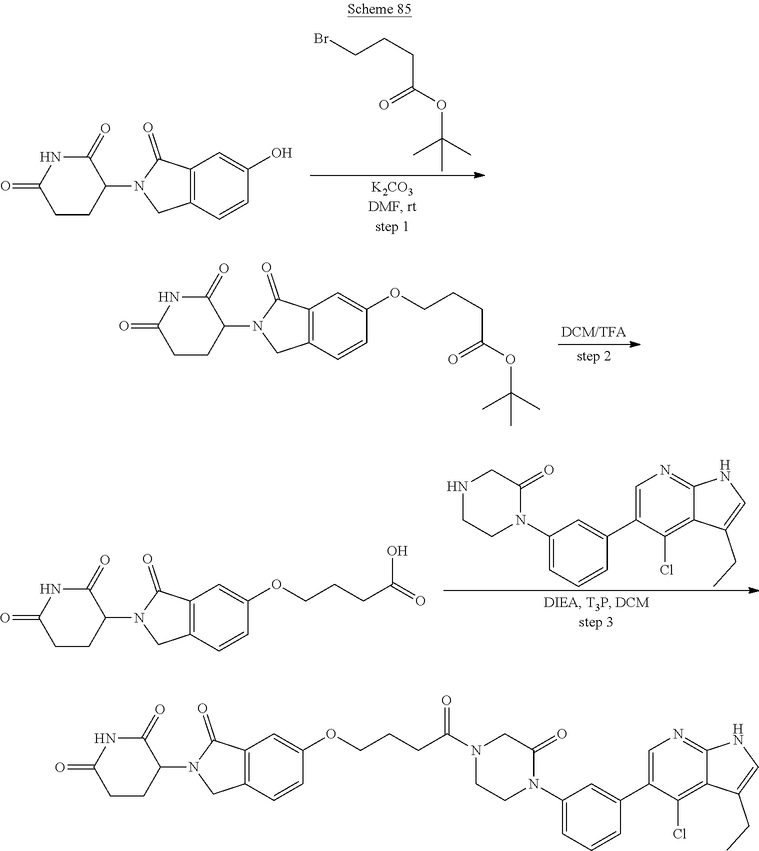 [SMILES: O=C1CCC(N2Cc3ccc(O)cc3C2=O)C(=O)N1 ; CC(C)(C)OC(=O)CCCBr ; CC(C)(C)OC(=O)CCCOc1ccc2c(c1)C(=O)N(C1CCC(=O)NC1=O)C2 ; O=C(O)CCCOc1ccc2c(c1)C(=O)N(C1CCC(=O)NC1=O)C2 ; CCc1c[nH]c2ncc(-c3cccc(N4CCNCC4=O)c3)c(Cl)c12 ; CCc1c[nH]c2ncc(-c3cccc(N4CCN(C(=O)CCCOc5ccc6c(c5)C(=O)N(C5CCC(=O)NC5=O)C6)CC4=O)c3)c(Cl)c12]

Step 1. Preparation of tert-butyl 4-((2-(2,6-dioxopiperidin-3-yl)-3-oxoisoindolin-5-yl)oxy) butanoate: Following General step E2, the desired product was obtained as white solid (50 mg, 15%). Mass (m/z): 403.2 [M+H]⁺.

Step 2. Preparation of 4-{[2-(2,6-dioxopiperidin-3-yl)-3-oxo-1H-isoindol-5-yl]oxy}butanoic acid: Following General step B2, the product 4-{[2-(2,6-dioxopiperidin-3-yl)-3-oxo-1H-isoindol-5-yl]oxy}butanoic acid (34 mg, 93%) was obtained as colorless oil. Mass (m/z): 347.0 [M+H]⁺.

Step 3. Preparation of 3-(6-{4-[4-(3-{4-chloro-3-ethyl-1H-pyrrolo[2,3-b]pyridin-3-yl}phenyl)-3-oxopiperazin-1-yl]-4-hydroxybutoxy}-1-oxo-3H-isoindol-2-yl)piperidine-2,6-dione: Following General step F, the product 3-(6-{4-[4-(3-{4-chloro-3-ethyl-1H-pyrrolo[2,3-b]pyridin-3-yl}phenyl)-3-oxopiperazin-1-yl]-4-hydroxybutoxy}-1-oxo-3H-isoindol-2-yl)piperidine-2,6-dione (2.00 mg, 3%) was obtained as a white solid. Mass (m/z): 683.0 [M+H]⁺. ¹H NMR (400 MHZ, CD₃OD) δ 8.10 (s, 1H), 7.56-7.44 (m, 4H), 7.39-7.26 (m, 3H), 7.16-7.12 (m, 1H), 7.02-6.99 (m, 1H), 4.43-4.35 (m, 3H), 4.15 (t, J=5.8 Hz, 1H), 3.99 (d, J=4.2 Hz, 2H), 3.91-3.88 (m, 4H), 3.00 (dd, J=14.8, 7.4 Hz, 2H), 2.92-2.85 (m, 2H), 2.74-2.69 (m, 1H), 2.66 (s, 1H), 2.54-2.44 (m, 2H), 2.21-2.15 (m, 2H), 1.87-1.98 (m, 2H), 1.33 (t, J=7.4 Hz, 3H).

### Example 68: 3-(6-((6-(4-(3-(4-chloro-3-ethyl-1H-pyrrolo[2,3-b]pyridin-5-yl)phenyl)-3-oxopiperazin-1-yl)-6-oxohexyl)oxy)-1-oxoisoindolin-2-yl)piperidine-2,6-dione

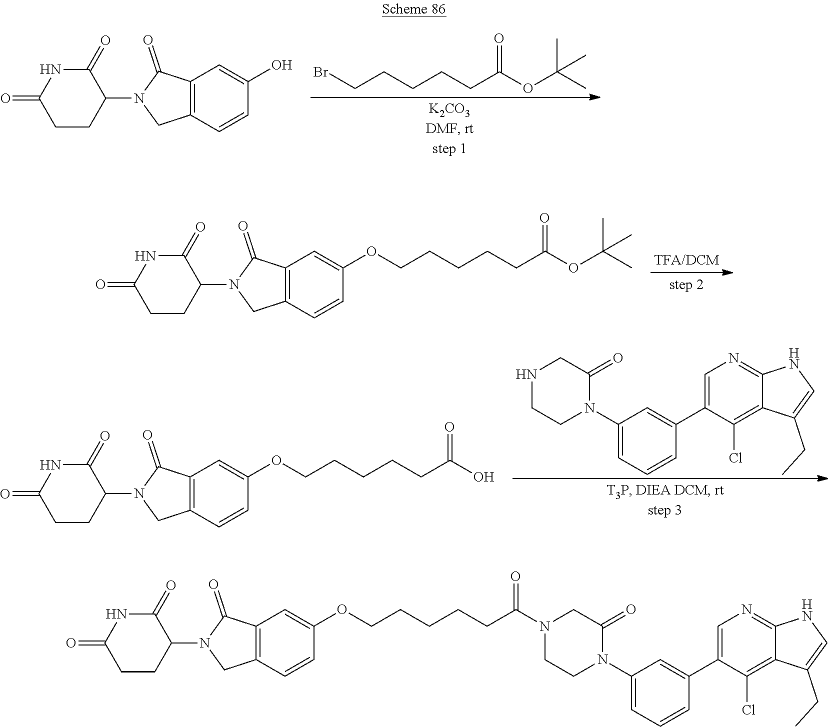 [SMILES: O=C1CCC(N2Cc3ccc(O)cc3C2=O)C(=O)N1 ; CC(C)(C)OC(=O)CCCCCBr ; CC(C)(C)OC(=O)CCCCCOc1ccc2c(c1)C(=O)N(C1CCC(=O)NC1=O)C2 ; O=C(O)CCCCCOc1ccc2c(c1)C(=O)N(C1CCC(=O)NC1=O)C2 ; CCc1c[nH]c2ncc(-c3cccc(N4CCNCC4=O)c3)c(Cl)c12 ; CCc1c[nH]c2ncc(-c3cccc(N4CCN(C(=O)CCCCCOc5ccc6c(c5)C(=O)N(C5CCC(=O)NC5=O)C6)CC4=O)c3)c(Cl)c12]

Step 1. Preparation of tert-butyl 6-((2-(2,6-dioxopiperidin-3-yl)-3-oxoisoindolin-5-yl)oxy) hexanoate: Following General step E2, the desired product was obtained as white solid (50 mg, 15.1%). Mass (m/z): 431.2 [M+H]⁺.

Step 2. Preparation of 6-((2-(2,6-dioxopiperidin-3-yl)-3-oxoisoindolin-5-yl)oxy) hexanoic acid: Following General step B2, the product 6-((2-(2,6-dioxopiperidin-3-yl)-3-oxoisoindolin-5-yl)oxy) hexanoic acid was obtained as yellow oil (40 mg, purity: 60%). Mass (m/z): 375.0 [M+H]⁺.

Step 3. Preparation of 3-(6-((6-(4-(3-(4-chloro-3-ethyl-1H-pyrrolo[2,3-b]pyridin-5-yl)phenyl)-3-oxopiperazin-1-yl)-6-oxohexyl)oxy)-1-oxoisoindolin-2-yl)piperidine-2,6-dione: Following General step F, the product 3-(6-((6-(4-(3-(4-chloro-3-ethyl-1H-pyrrolo[2,3-b]pyridin-5-yl)phenyl)-3-oxopiperazin-1-yl)-6-oxohexyl)oxy)-1-oxoisoindolin-2-yl)piperidine-2,6-dione was obtained as a white solid (30 mg, 40%). Mass (m/z): 738.7 [M+H]⁺. ¹H NMR (400 MHz, DMSO-d₆) δ 11.80 (s, 1H), 9.81 (s, 1H), 8.12 (s, 1H), 7.69 (d, J=13.2 Hz, 1H), 7.50 (dd, J=14.6, 6.8 Hz, 2H), 7.40 (t, J=7.6 Hz, 3H), 7.02 (d, J=11.2 Hz, 2H), 5.14 (d, J=8.4 Hz, 1H), 4.38-4.27 (m, 2H), 4.24-4.15 (m, 2H), 3.89-3.74 (m, 4H), 3.68-3.57 (m, 2H), 3.08-2.87 (m, 4H), 2.75 (s, 2H), 2.41-2.32 (m, 3H), 2.00 (d, J=10.4 Hz, 1H), 1.62-1.43 (m, 4H), 1.27 (s, 3H).

### Example 69: 1-(6-(4-(3-(4-(6-(4-chloro-3-cyclopropyl-1H-pyrrolo[2,3-b]pyridin-5-yl)pyridin-2-yl)-3-oxopiperazin-1-yl) propyl)piperazin-1-yl) naphthalen-1-yl)dihydropyrimidine-2,4 (1H,3H)-dione

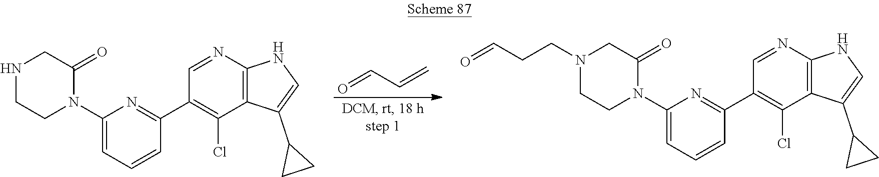

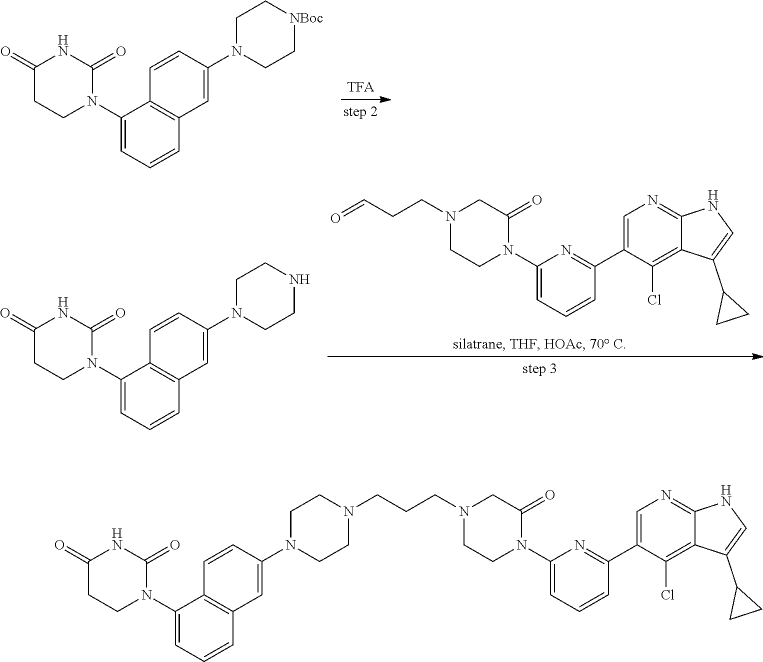 [SMILES: CC(C)(C)OC(=O)N1CCN(c2ccc3c(N4CCC(=O)NC4=O)cccc3c2)CC1 ; O=C1CCN(c2cccc3cc(N4CCNCC4)ccc23)C(=O)N1 ; O=CCCN1CCN(c2cccc(-c3cnc4[nH]cc(C5CC5)c4c3Cl)n2)C(=O)C1 ; O=C1CCN(c2cccc3cc(N4CCN(CCCN5CCN(c6cccc(-c7cnc8[nH]cc(C9CC9)c8c7Cl)n6)C(=O)C5)CC4)ccc23)C(=O)N1]

Step 1. Preparation of 3-(4-(6-(4-chloro-3-cyclopropyl-1H-pyrrolo[2,3-b]pyridin-5-yl)pyridin-2-yl)-3-oxopiperazin-1-yl) propanal: A solution of 1-(6-{4-chloro-3-cyclopropyl-1H-pyrrolo[2,3-b]pyridin-3-yl}pyridin-2-yl)piperazin-2-one (500 mg, 1.35 mmol) and prop-2-enal (762 mg, 13.59 mmol) in DCM (10 mL) was stirred at rt for 18 hrs. The solution was concentrated under reduced pressure to give the desired product as a yellow solid (500 mg, 82%) as a yellow solid. Mass (m/z): 424 [M+H]⁺.

Step 2. Preparation of 1-(6-(piperazin-1-yl) naphthalen-1-yl)dihydropyrimidine-2,4 (1H,3H)-dione: Following General step B2, the product 1-(6-(piperazin-1-yl) naphthalen-1-yl)dihydropyrimidine-2,4 (1H,3H)-dione was obtained as brown solid (434 mg, 52%). Mass (m/z): 325.0 [M+H]⁺.

Step 3. Preparation of 1-(6-(4-(3-(4-(6-(4-chloro-3-cyclopropyl-1H-pyrrolo[2,3-b]pyridin-5-yl)pyridin-2-yl)-3-oxopiperazin-1-yl) propyl)piperazin-1-yl) naphthalen-1-yl)dihydropyrimidine-2,4 (1H,3H)-dione: Following General step M2, the product was obtained as a white solid (14 mg, 6%). Mass (m/z): 733.1 [M+H]⁺. ¹H NMR (400 MHZ, DMSO-d₆) δ 11.84 (s, 1H), 10.41 (s, 1H), 8.38 (s, 1H), 8.32 (s, 1H), 7.94 (d, J=4.4 Hz, 2H), 7.73 (dd, J=12.2, 8.8 Hz, 2H), 7.56 (t, J=4.2 Hz, 1H), 7.44-7.38 (m, 2H), 7.32 (d, J=1.6 Hz, 1H), 7.26-7.21 (m, 2H), 4.01-3.82 (m, 4H), 3.67-3.58 (m, 2H), 3.29 (s, 2H), 2.94 (s, 2H), 2.85-2.79 (m, 2H), 2.75-2.65 (m, 2H), 2.57 (s, 3H), 2.54 (s, 1H), 2.46 (d, J=7.4 Hz, 2H), 2.40 (t, J=7.2 Hz, 2H), 2.22 (s, 1H), 1.69 (d, J=7.8 Hz, 2H), 0.89-0.84 (m, 2H), 0.68-0.63 (m, 2H).

### Example 70: 5-(1-(3-(4-(3-(4-chloro-3-cyclopropyl-1H-pyrrolo[2,3-b]pyridin-5-yl)phenyl)-3-oxopiperazin-1-yl) propyl)-1,2,3,6-tetrahydropyridin-4-yl)-2-(2,6-dioxopiperidin-3-yl)isoindoline-1,3-dione

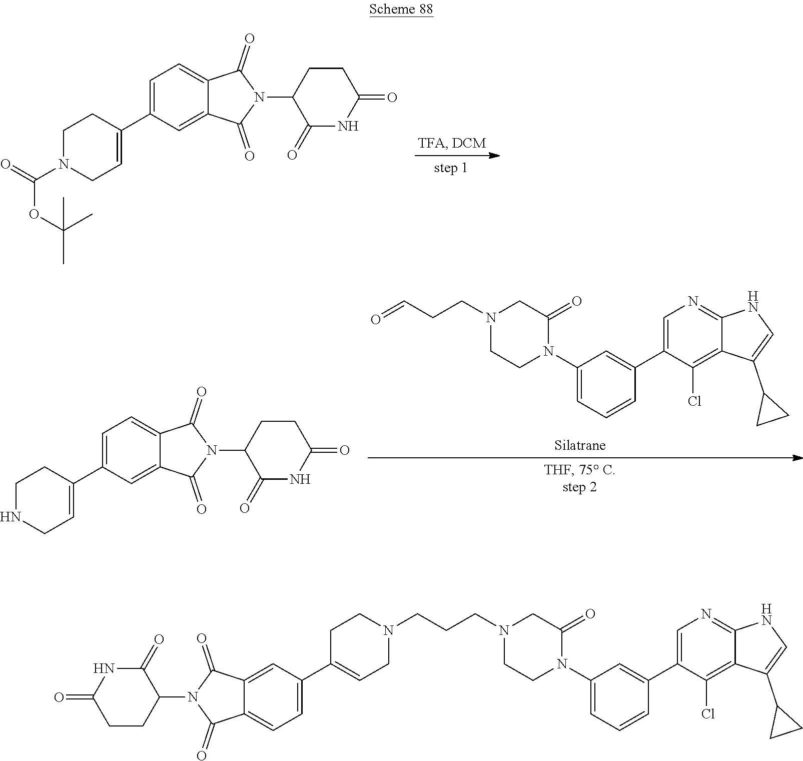 [SMILES: CC(C)(C)OC(=O)N1CC=C(c2ccc3c(c2)C(=O)N(C2CCC(=O)NC2=O)C3=O)CC1 ; O=C1CCC(N2C(=O)c3ccc(C4=CCNCC4)cc3C2=O)C(=O)N1 ; O=CCCN1CCN(c2cccc(-c3cnc4[nH]cc(C5CC5)c4c3Cl)c2)C(=O)C1 ; O=C1CCC(N2C(=O)c3ccc(C4=CCN(CCCN5CCN(c6cccc(-c7cnc8[nH]cc(C9CC9)c8c7Cl)c6)C(=O)C5)CC4)cc3C2=O)C(=O)N1]

Step 1. Preparation of 2-(2,6-dioxopiperidin-3-yl)-5-(1,2,3,6-tetrahydropyridin-4-yl) isoindoline-1,3-dione: Following General step B2, the desired product (350 mg, yield: 86.11%) was obtained as a brown solid. Mass (m/z): 339.9 [M+H]⁺.

Step 2. Preparation of 5-(1-(3-(4-(3-(4-chloro-3-cyclopropyl-1H-pyrrolo[2,3-b] pyridin-5-yl)phenyl)-3-oxopiperazin-1-yl) propyl)-1,2,3,6-tetrahydropyridin-4-yl)-2-(2,6-dioxopiperidin-3-yl)isoindoline-1,3-dione: Following General step M2, the desired product (10.5 mg, yield: 3.8%) was obtained as a white solid. Mass (m/z): 745.8 [M+H]⁺. ¹H NMR (400 MHz, DMSO-d₆) δ 11.78 (s, 1H), 11.15 (s, 1H), 8.12 (s, 1H), 8.07 (s, 1H), 8.01 (d, J=8.0 Hz, 1H), 7.95 (d, J=7.9 Hz, 1H), 7.52 (dd, J=16.8, 9.0 Hz, 2H), 7.41 (s, 2H), 7.32 (s, 1H), 6.59 (s, 1H), 5.18 (dd, J=12.5, 5.2 Hz, 1H), 4.02 (s, 2H), 3.84 (s, 3H), 3.54 (s, 4H), 3.29 (s, 3H), 2.89 (d, J=13.9 Hz, 3H), 2.62 (dd, J=29.6, 12.3 Hz, 2H), 2.21 (s, 2H), 2.06 (s, 3H), 1.23 (s, 1H), 0.89-0.80 (m, 2H), 0.65 (d, J=5.0 Hz, 2H).

### Example 71: 5-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)-2-(2,6-dioxopiperidin-3-yl) isoindoline-1,3-dione

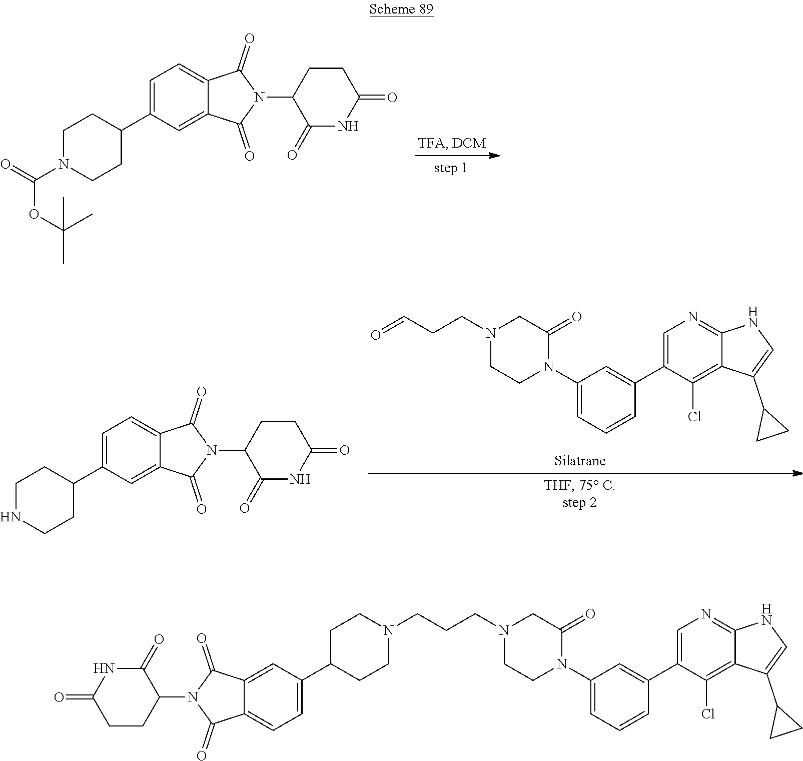 [SMILES: CC(C)(C)OC(=O)N1CCC(c2ccc3c(c2)C(=O)N(C2CCC(=O)NC2=O)C3=O)CC1 ; O=C1CCC(N2C(=O)c3ccc(C4CCNCC4)cc3C2=O)C(=O)N1 ; O=CCCN1CCN(c2cccc(-c3cnc4[nH]cc(C5CC5)c4c3Cl)c2)C(=O)C1 ; O=C1CCC(N2C(=O)c3ccc(C4CCN(CCCN5CCN(c6cccc(-c7cnc8[nH]cc(C9CC9)c8c7Cl)c6)C(=O)C5)CC4)cc3C2=O)C(=O)N1]

Step 1. 2-(2,6-dioxopiperidin-3-yl)-5-(piperidin-4-yl)isoindoline-1,3-dione: Following General step B2, the desired product (300 mg, yield: 81.91%) was obtained as a brown solid. Mass (m/z): 342 [M+H]⁺.

Step 2. 5-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)-2-(2,6-dioxopiperidin-3-yl)isoindoline-1,3-dione: Following General step M2, the desired product (30 mg, yield: 11.29%) was obtained as a white solid. Mass (m/z): 747.8 [M+H]⁺. ¹H NMR (400 MHZ, DMSO-d₆) δ 11.76 (d, J=2.3 Hz, 1H), 11.13 (s, 1H), 8.12 (s, 1H), 7.87 (d, J=7.8 Hz, 1H), 7.83-7.74 (m, 2H), 7.50 (dd, J=14.9, 7.1 Hz, 2H), 7.40 (t, J=8.2 Hz, 2H), 7.31 (d, J=1.6 Hz, 1H), 5.14 (dd, J=12.9, 5.4 Hz, 1H), 3.80-3.68 (m, 2H), 3.33 (s, 6H), 2.94-2.77 (m, 4H), 2.72-2.52 (m, 4H), 2.39 (s, 2H), 2.20 (dd, J=9.2, 4.0 Hz, 1H), 2.10-2.01 (m, 1H), 1.90 (d, J=7.2 Hz, 2H), 1.79 (d, J=10.5 Hz, 4H), 0.85 (dd, J=5.7, 4.0 Hz, 2H), 0.69-0.60 (m, 2H).

### Example 72: 3-cyclopropyl-1H-pyrrolo 1-(6-(4-(2-(4-(6-(4-chloro-3-cyclopropyl-1H-pyrrolo[2,3-b]pyridin-5-yl)pyridin-2-yl)-3-oxopiperazin-1-yl)ethoxy)piperidin-1-yl) naphthalen-1-yl)dihydropyrimidine-2,4 (1H,3H)-dione

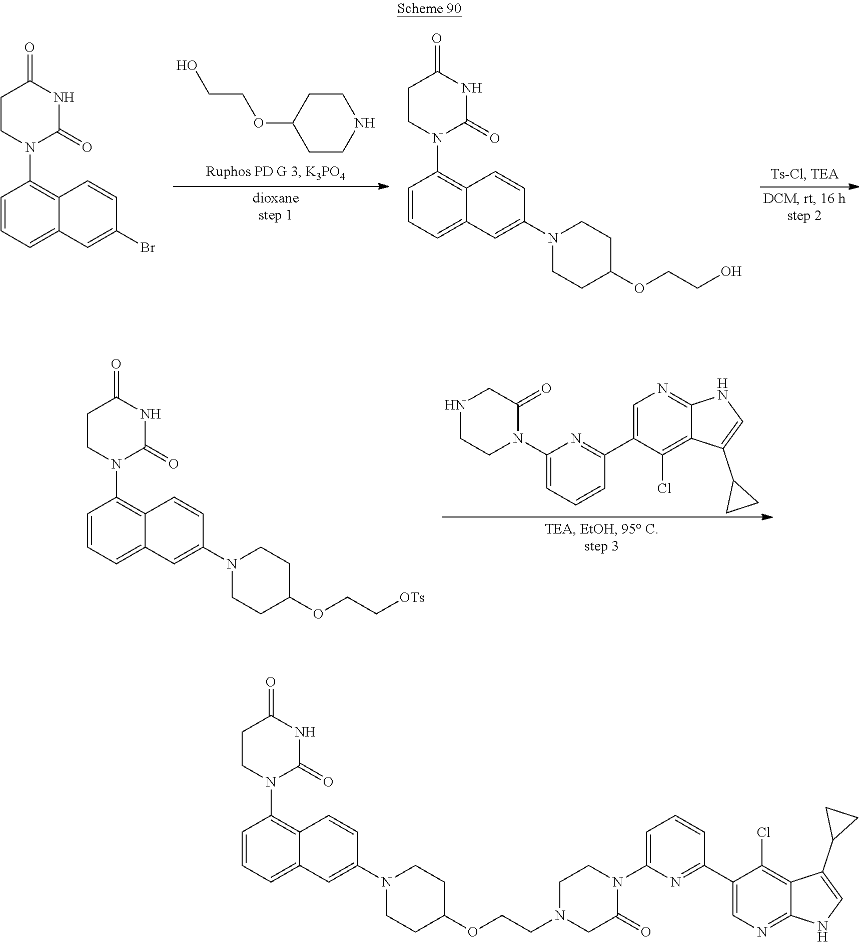 [SMILES: O=C1CCN(c2cccc3cc(Br)ccc23)C(=O)N1 ; OCCOC1CCNCC1 ; O=C1CCN(c2cccc3cc(N4CCC(OCCO)CC4)ccc23)C(=O)N1 ; Cc1ccc(S(=O)(=O)OCCOC2CCN(c3ccc4c(N5CCC(=O)NC5=O)cccc4c3)CC2)cc1 ; O=C1CNCCN1c1cccc(-c2cnc3[nH]cc(C4CC4)c3c2Cl)n1 ; O=C1CCN(c2cccc3cc(N4CCC(OCCN5CCN(c6cccc(-c7cnc8[nH]cc(C9CC9)c8c7Cl)n6)C(=O)C5)CC4)ccc23)C(=O)N1]

Step 1. Preparation of 1-{6-[4-(2-hydroxyethoxy)piperidin-1-yl]naphthalen-1-yl}-1,3-diazinane-2,4-dione: To a mixture of 1-(6-bromonaphthalen-1-yl)-1,3-diazinane-2,4-dione (50 mg, 0.157 mmol), K₃PO₄ (66 mg, 0.313 mmol) and 2-(piperidin-4-yloxy) ethanol (45 mg, 0.313 mmol) in dioxane (5 mL) was added Ruphos Pd G 3 (26 mg, 0.031 mmol). The reaction was degassed with N₂ for 3 times and stirred at 90° C. for 16 hrs. The reaction mixture was filtered and the filtrate was concentrated under reduced pressure. The residue was diluted with EA (25 mL) and H₂O (50 mL), then extracted with EA (50 mL×3). The organic layer was washed with brine (50 mL×2), dried over Na₂SO₄ and concentrated under reduced pressure. The residue was purified by Prep-TLC (DCM/MeOH=10:1) to give the product 1-{6-[4-(2-hydroxyethoxy)piperidin-1-yl]naphthalen-1-yl}-1,3-diazinane-2,4-dione (20 mg, 31%) as a yellow solid. Mass (m/z): 384.1 [M+H]⁺.

Step 2. Preparation of 2-({1-[5-(2,4-dioxo-1,3-diazinan-1-yl) naphthalen-2-yl] piperidin-4-yl}oxy)ethyl 4-methylbenzenesulfonate: To a mixture of 1-{6-[4-(2-hydroxyethoxy)piperidin-1-yl]naphthalen-1-yl}-1,3-diazinane-2,4-dione (130 mg, 0.339 mmol) in DCM (5 mL) was added TEA (103 mg, 1.02 mmol), 4-DMAP (4. mg, 0.033 mmol) and 4-methylbenzenesulfonyl chloride (96 mg, 0.509 mmol). The reaction was stirred at 25° C. under N₂ for 2 hrs. The reaction mixture was diluted with sat. NH₄Cl solution (20 mL), then extracted with EA (20 mL×3). The organic layer was washed with brine (50 mL), dried over Na₂SO₄ and concentrated under reduced pressure. The residue was purified by flash column (DCM/MeOH=0˜5%) to give the product 2-({1-[5-(2,4-dioxo-1,3-diazinan-1-yl) naphthalen-2-yl]piperidin-4-yl}oxy)ethyl 4-methylbenzenesulfonate as a yellow solid (70 mg, 70%). Mass (m/z): 538.2 [M+H]⁺.

Step 3. Preparation of 1-[6-(4-{2-[4-(6-{4-chloro-3-cyclopropyl-1H-pyrrolo[2,3-b]pyridin-3-yl}pyridin-2-yl)-3-oxopiperazin-1-yl]ethoxy}piperidin-1-yl) naphthalen-1-yl]-1,3-diazinane-2,4-dione: To a mixture of 2-({1-[5-(2,4-dioxo-1,3-diazinan-1-yl) naphthalen-2-yl]piperidin-4-yl}oxy)ethyl 4-methylbenzenesulfonate (50 mg, 0.093 mmol) in EtOH (5 mL) was added 1-(6-{4-chloro-3-cyclopropyl-1H-pyrrolo[2,3-b]pyridin-3-yl}pyridin-2-yl)piperazin-2-one (68 mg, 0.186 mmol) and TEA (28 mg, 0.279 mmol). The reaction was stirred at 95° C. under N₂ for 2 hrs. The mixture was concentrated under reduced pressure. The residue was purified by Prep-TLC (DCM/MeOH=10:1) to give the product 1-[6-(4-{2-[4-(6-{4-chloro-3-cyclopropyl-1H-pyrrolo[2,3-b]pyridin-3-yl}pyri dine-2-yl)-3-oxopiperazin-1-yl]ethoxy}piperidin-1-yl) naphthalen-1-yl]-1,3-diazinane-2,4-dione as a white solid (10 mg, 13%). Mass (m/z): 733.1 [M+H]⁺. ¹H NMR (400 MHZ, CDCl₃) δ 9.62 (s, 1H), 8.39 (s, 1H), 8.02 (d, J=8.2 Hz, 1H), 7.78 (t, J=7.8 Hz, 1H), 7.70-7.67 (m, 2H), 7.51 (d, J=7.4 Hz, 1H), 7.41 (t, J=7.8 Hz, 1H), 7.36 (d, J=7.8 Hz, 1H), 7.21 (d, J=6.0 Hz, 1H), 7.03 (s, 1H), 4.31-4.21 (m, 2H), 3.99-3.92 (m, 1H), 3.88-3.82 (m, 1H), 3.75 (d, J=6.8 Hz, 2H), 3.59 (s, 2H), 3.10 (s, 2H), 3.04-2.91 (m, 2H), 2.87 (d, J=6.2 Hz, 1H), 2.27-2.25 (m, 2H), 2.11-2.01 (m, 2H), 1.75-1.68 (m, 4H), 0.96-0.91 (m, 2H), 0.86-0.90 (m, 4H), 0.65 (d, J=3.8 Hz, 2H).

### Example 73: 3-(4-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)phenyl)piperidine-2,6-dione

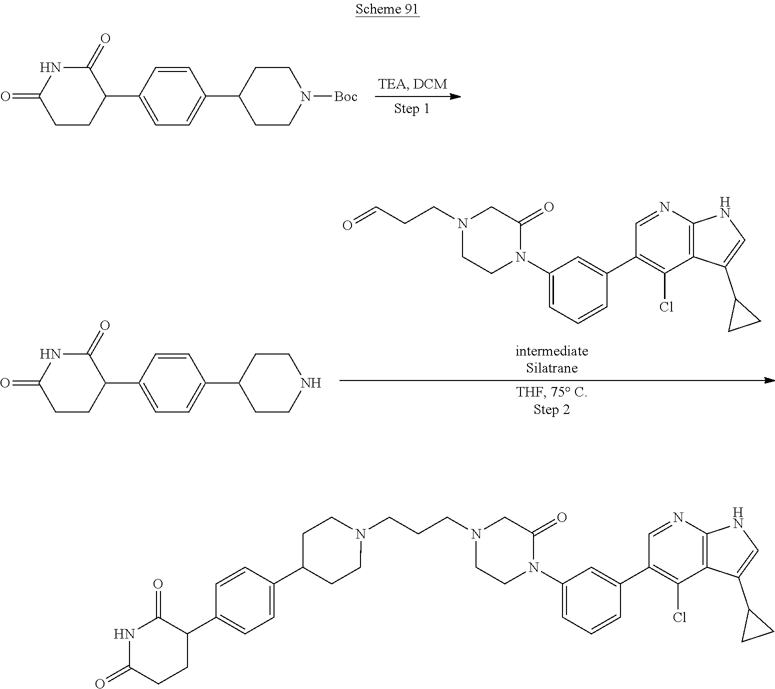 [SMILES: CC(C)(C)OC(=O)N1CCC(c2ccc(C3CCC(=O)NC3=O)cc2)CC1 ; O=C1CCC(c2ccc(C3CCNCC3)cc2)C(=O)N1 ; O=CCCN1CCN(c2cccc(-c3cnc4[nH]cc(C5CC5)c4c3Cl)c2)C(=O)C1 ; O=C1CCC(c2ccc(C3CCN(CCCN4CCN(c5cccc(-c6cnc7[nH]cc(C8CC8)c7c6Cl)c5)C(=O)C4)CC3)cc2)C(=O)N1]

Step 1. Preparation of 3-(4-(piperidin-4-yl)phenyl)piperidine-2,6-dione: Following General step B2, the product (120 mg, 78.17%) was obtained as brown solid. Mass (m/z): 273 [M+H]⁺.

Step 2. Preparation of 3-(4-(1-(3-(4-(3-(4-chloro-3-cyclopropyl-1H-pyrrolo[2,3-b] pyridin-5-yl)phenyl)-3-oxopiperazin-1-yl) propyl)piperidin-4-yl)phenyl)piperidine-2,6-dione: Following General step M2, the desired product (20 mg, yield: 6.56%) was obtained as a white solid. Mass (m/z): 678.8 [M+H]⁺. ¹H NMR (400 MHZ, DMSO-d₆) δ 11.74 (d, J=1.9 Hz, 1H), 10.81 (s, 1H), 8.08 (s, 1H), 7.46 (dd, J=14.9, 7.1 Hz, 2H), 7.36 (t, J=7.6 Hz, 2H), 7.28 (d, J=1.7 Hz, 1H), 7.17 (d, J=8.2 Hz, 2H), 7.11 (d, J=8.2 Hz, 2H), 3.77 (dd, J=11.5, 4.9 Hz, 1H), 3.71 (t, J=5.0 Hz, 2H), 3.19 (d, J=14.6 Hz, 4H), 2.77 (t, J=5.0 Hz, 2H), 2.70-2.51 (m, 4H), 2.43 (d, J=7.0 Hz, 5H), 2.15 (m, 2H), 2.02-1.92 (m, 1H), 1.81 (d, J=12.3 Hz, 2H), 1.76-1.62 (m, 4H), 0.84-0.75 (m, 2H), 0.64-0.55 (m, 2H).

### Example 74: 1-(6-(1-(3-(4-(3-(4-chloro-3-ethyl-1H-pyrrolo[2,3-b]pyridin-5-yl)phenyl)-3-oxopiperazin-1-yl) propyl)-1,2,3,6-tetrahydropyridin-4-yl)-1-methyl-1H-indazol-3-yl)dihydropyrimidine-2,4 (1H,3H)-dione

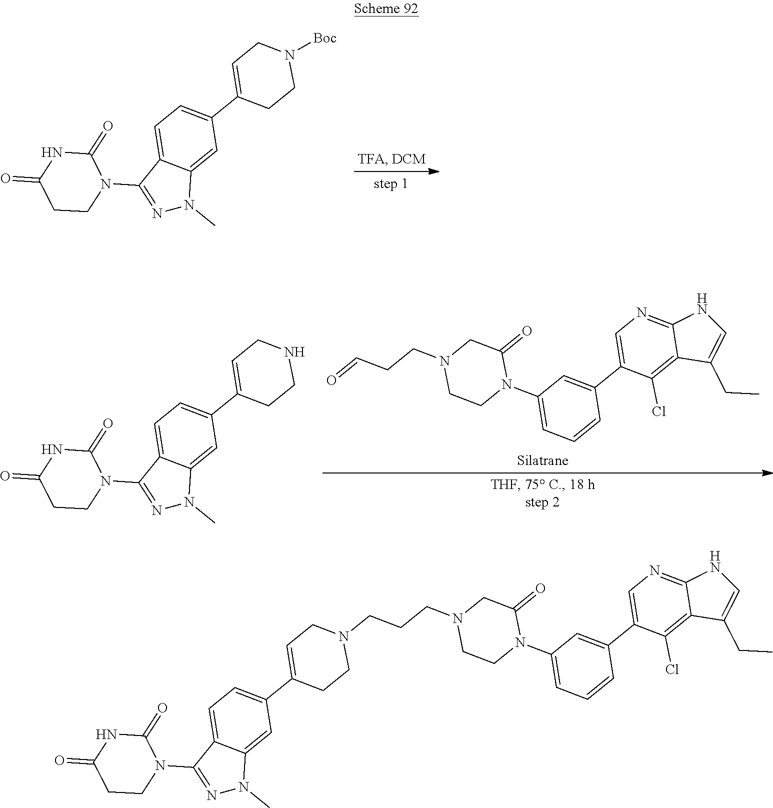

Step 1. Preparation of 1-(1-methyl-6-(1,2,3,6-tetrahydropyridin-4-yl)-1H-indazol-3-yl)dihydropyrimidine-2,4 (1H,3H)-dione: Following General step B2, the product (100 mg, 98.91%) was obtained as brown solid. Mass (m/z): 325.9 [M+H]⁺.

Step 2. Preparation of 1-(6-(1-(3-(4-(3-(4-chloro-3-ethyl-1H-pyrrolo[2,3-b]pyridin-5-yl)phenyl)-3-oxopiperazin-1-yl) propyl)-1,2,3,6-tetrahydropyridin-4-yl)-1-methyl-1H-indazol-3-yl)dihydropyrimidine-2,4 (1H,3H)-dione: Following General step M2, the desired product (30 mg, yield: 12.46%) was obtained as a white solid. Mass (m/z): 719.8 [M+H]⁺. ¹H NMR (400 MHz, DMSO-d₆) δ 11.77 (d, J=1.8 Hz, 1H), 10.54 (s, 1H), 8.09 (d, J=6.2 Hz, 1H), 7.56 (d, J=8.6 Hz, 2H), 7.45 (dd, J=15.1, 7.4 Hz, 2H), 7.35 (t, J=9.1 Hz, 3H), 7.26 (d, J=9.3 Hz, 1H), 6.29 (s, 1H), 3.96 (s, 3H), 3.88 (t, J=6.7 Hz, 2H), 3.71 (t, J=5.0 Hz, 2H), 3.18 (s, 2H), 2.88 (dd, J=14.7, 7.4 Hz, 3H), 2.82-2.75 (m, 2H), 2.72 (t, J=6.7 Hz, 3H), 2.66 (s, 3H), 1.76 (s, 2H), 1.23 (t, J=7.4 Hz, 3H).

### Example 75: 3-(4-(4-(3-(4-(3-(4-chloro-3-cyclopropyl-1H-pyrrolo[2,3-b]pyridin-5-yl)phenyl)-3-oxopiperazin-1-yl) propyl)piperazin-1-yl)phenyl)piperidine-2,6-dione

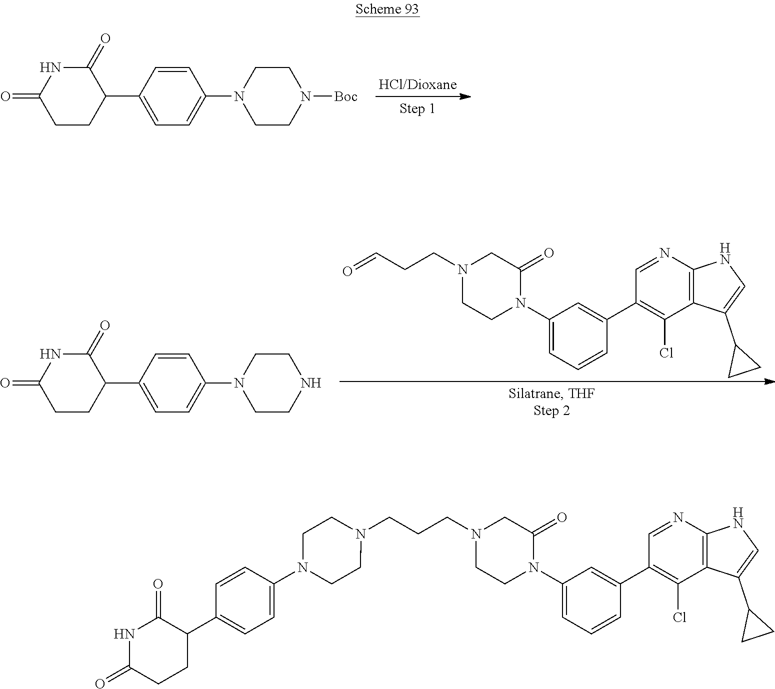 [SMILES: CC(C)(C)OC(=O)N1CCN(c2ccc(C3CCC(=O)NC3=O)cc2)CC1 ; O=C1CCC(c2ccc(N3CCNCC3)cc2)C(=O)N1 ; O=CCCN1CCN(c2cccc(-c3cnc4[nH]cc(C5CC5)c4c3Cl)c2)C(=O)C1 ; O=C1CCC(c2ccc(N3CCN(CCCN4CCN(c5cccc(-c6cnc7[nH]cc(C8CC8)c7c6Cl)c5)C(=O)C4)CC3)cc2)C(=O)N1]

Step 1. Preparation of 3-(4-(piperazin-1-yl)phenyl)piperidine-2,6-dione: To a solution of tert-butyl {4-[4-(2,6-dioxopiperidin-3-yl)phenyl]piperazin-1-yl}formate (2.2 g, 5.87 mmol) in DCM (10 mL) was added 4 M HCl/dioxane (14.7 mL, 58.7 mmol). The reaction mixture was stirred at 25° C. for 2 hrs. The reaction mixture was concentrated and freeze dried to give the product as a white solid (2 g, 99%). Mass (m/z): 274.2 [M+H]⁺.

Step 2. Preparation of 3-(4-(4-(3-(4-(3-(4-chloro-3-cyclopropyl-1H-pyrrolo[2,3-b]pyridin-5-yl)phenyl)-3-oxopiperazin-1-yl) propyl)piperazin-1-yl)phenyl)piperidine-2,6-dione: Following General step M2, the product was obtained as a white solid (60 mg, 12%). Mass (m/z): 679.8 [M+H]⁺. ¹H NMR (400 MHz, DMSO-d₆) δ 11.77 (d, J=2.2 Hz, 1H), 10.78 (s, 1H), 8.12 (s, 1H), 7.53-7.46 (m, 2H), 7.43-7.37 (m, 2H), 7.31 (d, J=1.8 Hz, 1H), 7.04 (d, J=8.8 Hz, 2H), 6.88 (d, J=8.8 Hz, 2H), 3.76-3.69 (m, 3H), 3.19 (s, 2H), 3.14-3.06 (m, 4H), 2.84-2.73 (m, 2H), 2.69-2.58 (m, 1H), 2.54-2.52 (m, 2H), 2.48-2.34 (m, 7H), 2.23-2.18 (m, 1H), 2.15-2.07 (m, 1H), 2.02-1.96 (m, 1H), 1.72-1.64 (m, 2H), 0.87-0.82 (m, 2H), 0.67-0.62 (m, 2H).

### Example 76: 1-(6-(4-(3-(4-(3-(4-chloro-3-ethyl-1H-pyrrolo[2,3-b]pyridin-5-yl)phenyl)-3-oxopiperazin-1-yl) propyl)piperazin-1-yl) naphthalen-1-yl)dihydropyrimidine-2,4 (1H,3H)-dione

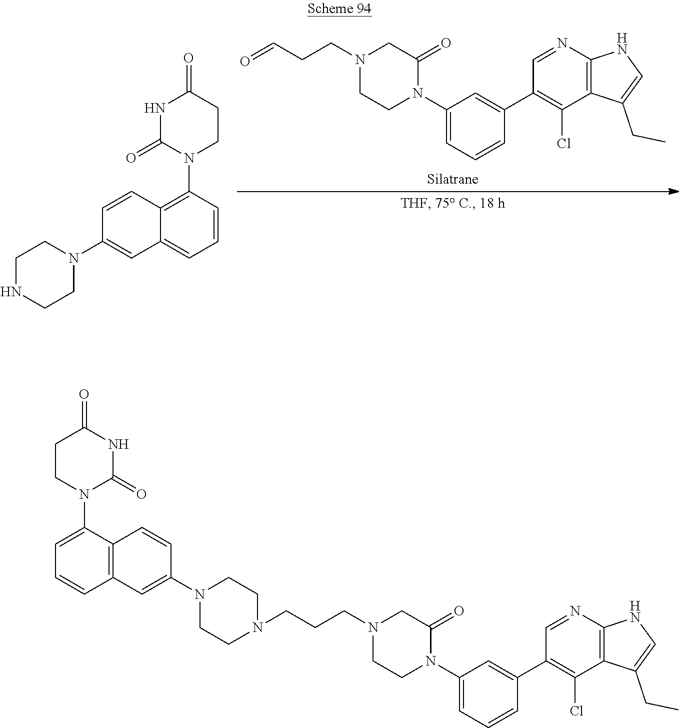 [SMILES: O=C1CCN(c2cccc3cc(N4CCNCC4)ccc23)C(=O)N1 ; CCc1c[nH]c2ncc(-c3cccc(N4CCN(CCC=O)CC4=O)c3)c(Cl)c12 ; CCc1c[nH]c2ncc(-c3cccc(N4CCN(CCCN5CCN(c6ccc7c(N8CCC(=O)NC8=O)cccc7c6)CC5)CC4=O)c3)c(Cl)c12]

Step 1. Preparation of 1-(6-(4-(3-(4-(3-(4-chloro-3-ethyl-1H-pyrrolo[2,3-b]pyridin-5-yl)phenyl)-3-oxopiperazin-1-yl) propyl)piperazin-1-yl) naphthalen-1-yl)dihydropyrimidine-2,4 (1H,3H)-dione: Following General step M2, the desired product (7 mg, yield: 3.08%) was obtained as a white solid. Mass (m/z): 719.3 [M+H]⁺. ¹H NMR (400 MHZ, DMSO-d₆) δ 11.80 (d, J=2.2 Hz, 1H), 10.42 (s, 1H), 8.13 (d, J=5.0 Hz, 1H), 7.75 (dd, J=15.4, 8.8 Hz, 2H), 7.54-7.35 (m, 7H), 7.27 (d, J=7.6 Hz, 2H), 3.86 (dd, J=6.1, 4.0 Hz, 1H), 3.79-3.71 (m, 2H), 3.70-3.56 (m, 2H), 3.22 (s, 3H), 2.93 (dt, J=14.7, 6.9 Hz, 4H), 2.88-2.76 (m, 4H), 2.76-2.64 (m, 3H), 2.62-2.51 (m, 3H), 2.48 (s, 1H), 2.33 (s, 1H), 1.78 (s, 2H), 1.28 (t, J=7.4 Hz, 3H).

### Example 77: 1-(6-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)-1-methyl-1H-indazol-3-yl)dihydro pyrimidine-2,4 (1H,3H)-dione

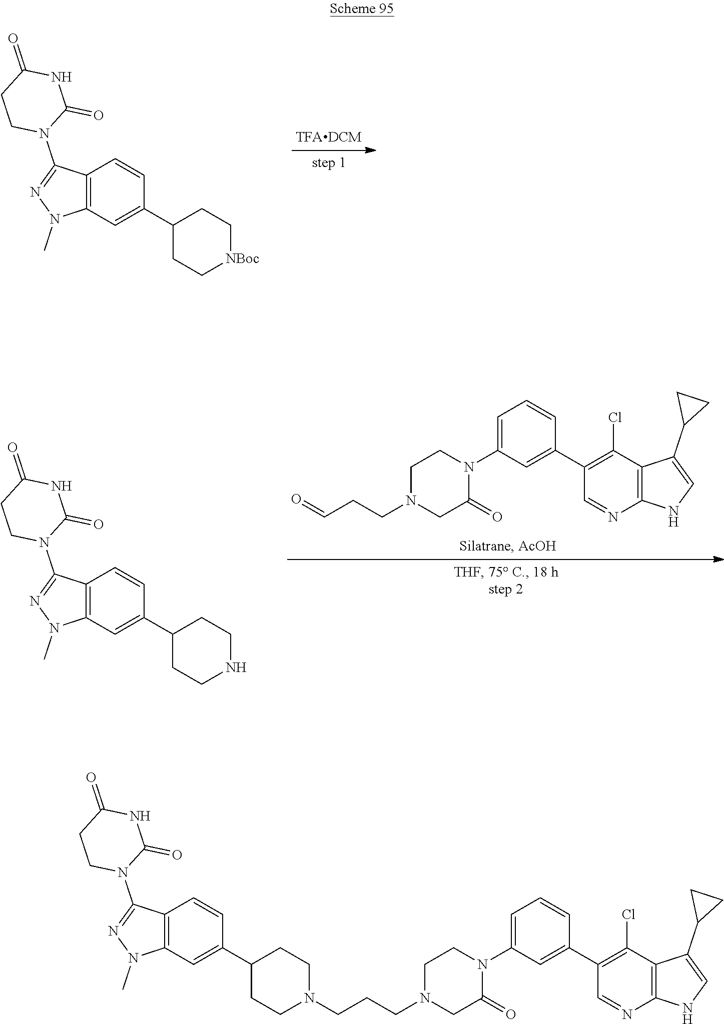 [SMILES: Cn1nc(N2CCC(=O)NC2=O)c2ccc(C3CCN(C(=O)OC(C)(C)C)CC3)cc21 ; Cn1nc(N2CCC(=O)NC2=O)c2ccc(C3CCNCC3)cc21 ; O=CCCN1CCN(c2cccc(-c3cnc4[nH]cc(C5CC5)c4c3Cl)c2)C(=O)C1 ; Cn1nc(N2CCC(=O)NC2=O)c2ccc(C3CCN(CCCN4CCN(c5cccc(-c6cnc7[nH]cc(C8CC8)c7c6Cl)c5)C(=O)C4)CC3)cc21]

Step 1. Preparation of 1-(1-methyl-6-(piperidin-4-yl)-1H-indazol-3-yl)dihydropyrimidine-2,4 (1H,3H)-dione: From tert-butyl 4-[3-(2,4-dioxo-1,3-diazinan-1-yl)-1-methylindazol-6-yl]piperidine-1-carboxylate (86 mg, 0.20 mmol), following General step B2, the desired product was obtained by suction filtration (150 mg, 51%) as a brown solid. Mass (m/z): 328 [M+H]⁺.

Step 2. Preparation of 1-(6-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)-1-methyl-1H-indazol-3-yl)dihydropyrimidine-2,4 (1H,3H)-dione: From 1-[1-methyl-6-(piperidin-4-yl) indazol-3-yl]-1,3-diazinane-2,4-dione (70 mg, 0.21 mmol) and 3-[4-(3-{4-chloro-3-cyclopropyl-1H-pyrrolo[2,3-b]pyridin-3-yl}phenyl)-3-oxopiperazin-1-yl]propanal (90 mg, 0.21 mmol), following General step M2, the desired product (18 mg, 11%) was obtained as a white solid. Mass (m/z): 733.0 [M+H]⁺. ¹H NMR (400 MHZ, DMSO-d₆) δ 11.77 (s, 1H), 10.55 (s, 1H), 8.12 (s, 1H), 7.55 (d, J=8.4 Hz, 1H), 7.53-7.41 (m, 4H), 7.39 (d, J=7.8 Hz, 1H), 7.31 (d, J=1.8 Hz, 1H), 7.04 (d, J=8.4 Hz, 1H), 3.97 (s, 3H), 3.90 (t, J=6.6 Hz, 2H), 3.78-3.71 (m, 2H), 3.21 (s, 2H), 3.08 (d, J=10.6 Hz, 2H), 2.84-2.79 (m, 2H), 2.75 (t, J=6.6 Hz, 2H), 2.67 (s, 2H), 2.46 (d, J=7.0 Hz, 3H), 2.12 (s, 3H), 1.87-1.65 (m, 6H), 0.88-0.80 (m, 2H), 0.65 (d, J=3.8 Hz, 2H).

### Example 78: 3-((4-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)phenyl)amino)piperidine-2,6-dione

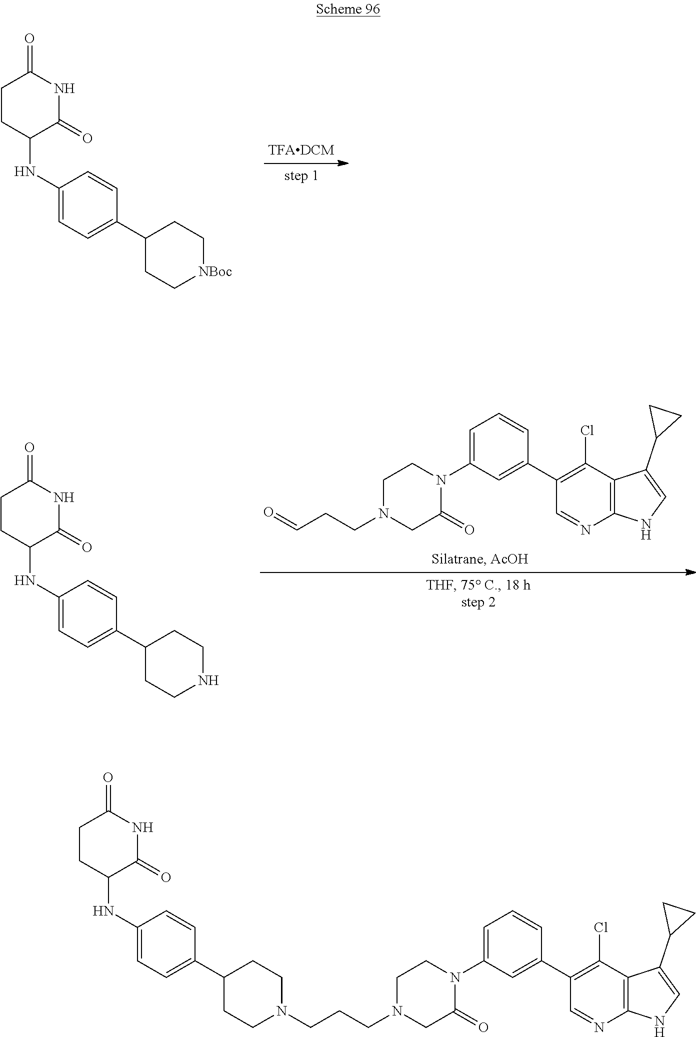 [SMILES: CC(C)(C)OC(=O)N1CCC(c2ccc(NC3CCC(=O)NC3=O)cc2)CC1 ; O=C1CCC(Nc2ccc(C3CCNCC3)cc2)C(=O)N1 ; O=CCCN1CCN(c2cccc(-c3cnc4[nH]cc(C5CC5)c4c3Cl)c2)C(=O)C1 ; O=C1CCC(Nc2ccc(C3CCN(CCCN4CCN(c5cccc(-c6cnc7[nH]cc(C8CC8)c7c6Cl)c5)C(=O)C4)CC3)cc2)C(=O)N1]

Step 1. Preparation of 3-((4-(piperidin-4-yl)phenyl)amino)piperidine-2,6-dione: From tert-butyl 4-{4-[(2,6-dioxopiperidin-3-yl)amino]phenyl}piperidine-1-carboxylate (301 mg, 0.77 mmol), following General step B2, the desired product was obtained by suction filtration (200 mg, 85%) as a brown solid. Mass (m/z): 288.1 [M+H]⁺.

Step 2. Preparation of 3-((4-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)phenyl)amino)piperidine-2,6-dione: From 3-{[4-(piperidin-4-yl)phenyl]amino}piperidine-2,6-dione (200 mg, 0.696 mmol) and 3-[4-(3-{4-chloro-3-cyclopropyl-1H-pyrrolo[2,3-b]pyridin-3-yl}phenyl)-3-oxopiperazin-1-yl]propanal (294 mg, 0.696 mmol), following General step M2, the desired product 3-((4-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)phenyl)amino)piperidine-2,6-dione (38 mg, 8%) was obtained as a white solid. Mass (m/z): 693.8 [M+H]⁺. ¹H NMR (400 MHz, DMSO-d₆) δ 11.77 (d, J=2.4 Hz, 1H), 10.78 (s, 1H), 8.12 (s, 1H), 7.56-7.45 (m, 2H), 7.40 (dd, J=9.6, 4.8 Hz, 2H), 7.31 (d, J=1.8 Hz, 1H), 6.96 (d, J=8.4 Hz, 2H), 6.62 (d, J=8.6 Hz, 2H), 5.71 (d, J=7.6 Hz, 1H), 4.31-4.21 (m, 1H), 3.79-3.70 (m, 2H), 3.22 (s, 3H), 2.88-2.64 (m, 5H), 2.63-2.52 (m, 3H), 2.42 (t, J=29.2 Hz, 4H), 2.20 (td, J=7.8, 3.8 Hz, 1H), 2.15-2.04 (m, 1H), 1.93-1.75 (m, 5H), 1.74-1.59 (m, 2H), 0.89-0.81 (m, 2H), 0.68-0.60 (m, 2H).

### Example 79: 3-((4-(4-(3-(4-(3-(4-chloro-3-cyclopropyl-1H-pyrrolo[2,3-b]pyridin-5-yl)phenyl)-3-oxopiperazin-1-yl) propyl)piperazin-1-yl)-3-fluorophenyl)amino) piperidine-2,6-dione

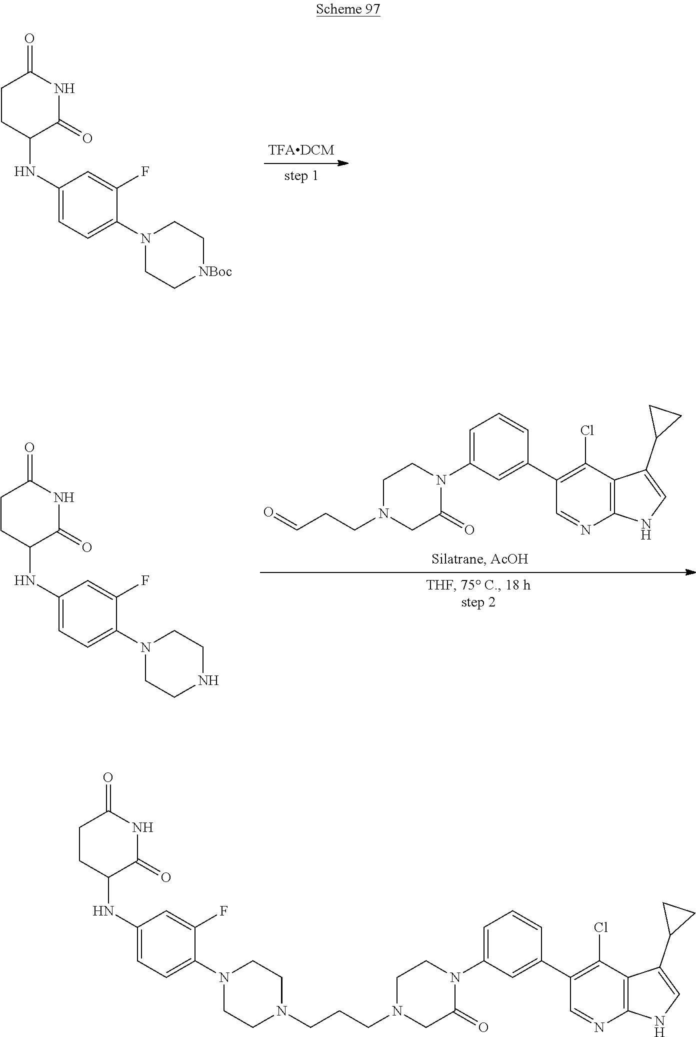 [SMILES: CC(C)(C)OC(=O)N1CCN(c2ccc(NC3CCC(=O)NC3=O)cc2F)CC1 ; O=C1CCC(Nc2ccc(N3CCNCC3)c(F)c2)C(=O)N1 ; O=CCCN1CCN(c2cccc(-c3cnc4[nH]cc(C5CC5)c4c3Cl)c2)C(=O)C1 ; O=C1CCC(Nc2ccc(N3CCN(CCCN4CCN(c5cccc(-c6cnc7[nH]cc(C8CC8)c7c6Cl)c5)C(=O)C4)CC3)c(F)c2)C(=O)N1]

Step 1. Preparation of 3-((3-fluoro-4-(piperazin-1-yl)phenyl)amino)piperidine-2,6-dione: From tert-butyl 4-{4-[(2,6-dioxopiperidin-3-yl)amino]-2-fluorophenyl}piperazine-1-carboxylate (431 mg, 1.06 mmol), following General step B2, the desired product was obtained by suction filtration (300 mg, 87%) as a brown solid. Mass (m/z): 307 [M+H]⁺.

Step 2. Preparation of 3-((4-(4-(3-(4-(3-(4-chloro-3-cyclopropyl-1H-pyrrolo[2,3-b] pyridin-5-yl)phenyl)-3-oxopiperazin-1-yl) propyl)piperazin-1-yl)-3-fluorophenyl)amino) piperidine-2,6-dione: From 3-{[3-fluoro-4-(piperazin-1-yl)phenyl]amino}piperidine-2,6-dione (200 mg, 0.65 mmol) and 3-[4-(3-{4-chloro-3-cyclopropyl-1H-pyrrolo[2,3-b]pyridin-3-yl}phenyl)-3-oxopiperazin-1-yl]propanal (276 mg, 0.65 mmol), following General step M2, the desired product (36 mg, 7%) was obtained as a white solid. Mass (m/z): 712.8 [M+H]⁺. ¹H NMR (400 MHz, DMSO-d₆) ¿ 11.76 (d, J=2.4 Hz, 1H), 10.79 (s, 1H), 8.13 (d, J=5.2 Hz, 1H), 7.55-7.46 (m, 2H), 7.40 (dd, J=10.6, 4.8 Hz, 2H), 7.31 (d, J=1.8 Hz, 1H), 6.86 (t, J=9.2 Hz, 1H), 6.53 (dd, J=15.0, 2.4 Hz, 1H), 6.43 (d, J=8.8 Hz, 1H), 5.88 (d, J=7.8 Hz, 1H), 4.26 (dd, J=8.2, 3.6 Hz, 1H), 3.80-3.70 (m, 2H), 3.22 (s, 3H), 2.98 (s, 6H), 2.85-2.66 (m, 5H), 2.62-2.51 (m, 3H), 2.48-2.44 (m, 1H), 2.25-2.15 (m, 1H), 2.07 (dd, J=8.2, 4.2 Hz, 1H), 1.92-1.70 (m, 3H), 0.85 (ddd, J=8.2, 6.0, 4.0 Hz, 2H), 0.69-0.60 (m, 2H).

### Example 80: 5-(4-(3-(4-(6-(4-chloro-3-cyclopropyl-1H-pyrrolo[2,3-b]pyridin-5-yl)pyridin-2-yl)-3-oxopiperazin-1-yl) propyl)piperazin-1-yl)-2-(2,6-dioxopiperidin-3-yl)isoindoline-1,3-dione

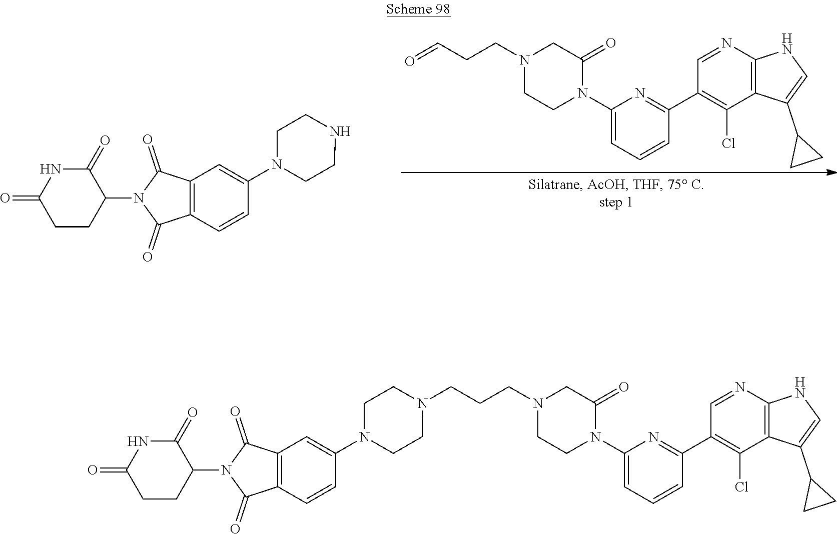 [SMILES: O=C1CCC(N2C(=O)c3ccc(N4CCNCC4)cc3C2=O)C(=O)N1 ; O=CCCN1CCN(c2cccc(-c3cnc4[nH]cc(C5CC5)c4c3Cl)n2)C(=O)C1 ; O=C1CCC(N2C(=O)c3ccc(N4CCN(CCCN5CCN(c6cccc(-c7cnc8[nH]cc(C9CC9)c8c7Cl)n6)C(=O)C5)CC4)cc3C2=O)C(=O)N1]

Step 1. Preparation of 5-(4-(3-(4-(6-(4-chloro-3-cyclopropyl-1H-pyrrolo[2,3-b]pyridin-5-yl)pyridin-2-yl)-3-oxopiperazin-1-yl) propyl)piperazin-1-yl)-2-(2,6-dioxopiperidin-3-yl)isoindoline-1,3-dione: Following General step M2, the product was obtained as a yellow solid (35 mg, 8%). Mass (m/z): 750.4 [M+H]⁺. ¹H NMR (400 MHZ, DMSO-d₆) δ 11.83 (s, 1H), 11.09 (s, 1H), 8.32 (s, 1H), 7.94 (d, J=4.0 Hz, 2H), 7.67 (d, J=8.4 Hz, 1H), 7.56 (t, J=4.2 Hz, 1H), 7.33 (d, J=5.4 Hz, 2H), 7.25 (d, J=8.6 Hz, 1H), 5.07 (dd, J=12.8, 5.2 Hz, 1H), 3.96 (s, 2H), 3.44 (s, 5H), 3.28 (s, 3H), 2.94-2.77 (m, 3H), 2.59 (d, J=16.4 Hz, 2H), 2.48-2.42 (m, 2H), 2.38 (t, J=6.8 Hz, 2H), 2.27-2.19 (m, 1H), 2.07-1.96 (m, 1H), 1.74-1.63 (m, 2H), 0.86 (d, J=8.2 Hz, 2H), 0.66 (d, J=5.0 Hz, 2H).

### Example 81: 3-(5-(4-(3-(4-(6-(4-chloro-3-cyclopropyl-1H-pyrrolo[2,3-b]pyridin-5-yl)pyridin-2-yl)-3-oxopiperazin-1-yl) propyl)piperazin-1-yl)-1-oxoisoindolin-2-yl)piperidine-2,6-dione

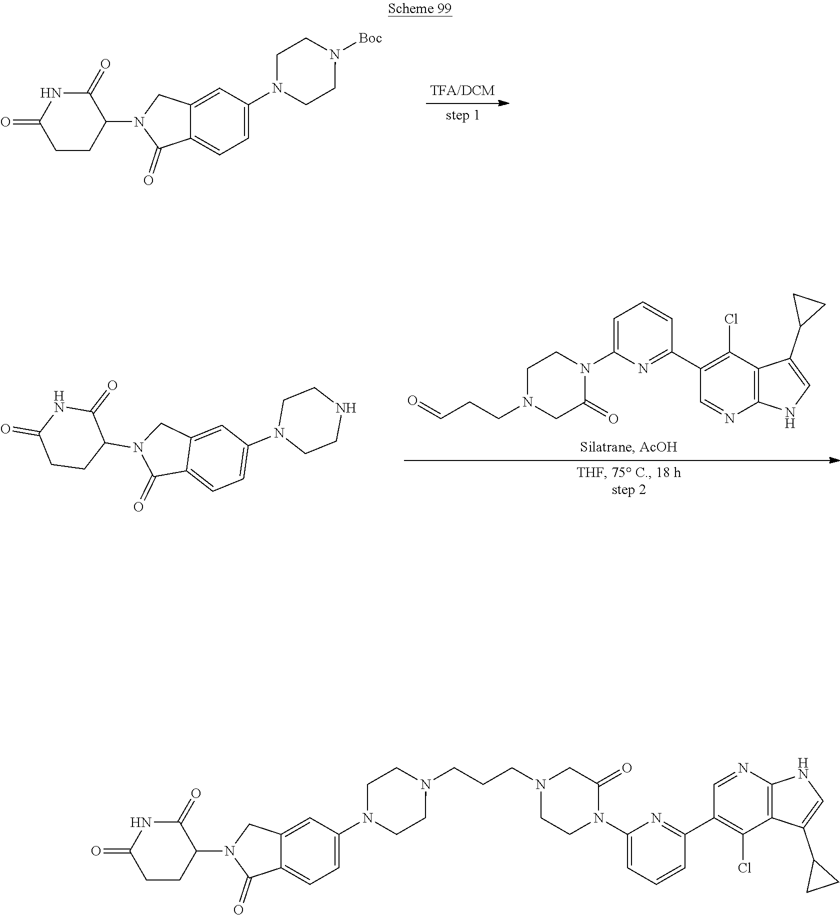 [SMILES: CC(C)(C)OC(=O)N1CCN(c2ccc3c(c2)CN(C2CCC(=O)NC2=O)C3=O)CC1 ; O=C1CCC(N2Cc3cc(N4CCNCC4)ccc3C2=O)C(=O)N1 ; O=CCCN1CCN(c2cccc(-c3cnc4[nH]cc(C5CC5)c4c3Cl)n2)C(=O)C1 ; O=C1CCC(N2Cc3cc(N4CCN(CCCN5CCN(c6cccc(-c7cnc8[nH]cc(C9CC9)c8c7Cl)n6)C(=O)C5)CC4)ccc3C2=O)C(=O)N1]

Step 1. Preparation of 3-(1-oxo-5-(piperazin-1-yl) isoindolin-2-yl)piperidine-2,6-dione: Following General step B2, the desired product was obtained by suction filtration (500 mg, 88%) as a brown solid. Mass (m/z): 329 [M+H]⁺.

Step 2. Preparation of 3-(5-(4-(3-(4-(6-(4-chloro-3-cyclopropyl-1H-pyrrolo[2,3-b] pyridin-5-yl)pyridine-2-yl)-3-oxopiperazin-1-yl) propyl)piperazin-1-yl)-1-oxoisoindolin-2-yl) piperidine-2,6-dione: Following General step M2, the desired product (22 mg, yield: 5%) was obtained as a white solid. Mass (m/z): 735.8 [M+H]⁺. ¹H NMR (400 MHZ, DMSO-d₆) δ 11.83 (d, J=2.0 Hz, 1H), 10.95 (s, 1H), 8.32 (s, 1H), 8.19 (s, 1H), 7.96-7.89 (m, 2H), 7.59-7.47 (m, 2H), 7.33 (d, J=1.6 Hz, 1H), 7.07 (s, 1H), 5.05 (dd, J=13.4, 5.0 Hz, 1H), 4.26 (dd, J=50.8, 16.8 Hz, 2H), 3.99-3.89 (m, 2H), 3.28 (s, 5H), 2.88 (d, J=13.2 Hz, 1H), 2.84-2.77 (m, 2H), 2.57 (d, J=23.4 Hz, 4H), 2.45 (d, J=7.2 Hz, 2H), 2.36 (dt, J=8.6, 5.6 Hz, 3H), 2.27-2.19 (m, 1H), 2.08 (s, 1H), 1.96 (d, J=5.5 Hz, 1H), 1.75-1.64 (m, 2H), 1.21 (s, 1H), 0.89-0.81 (m, 2H), 0.70-0.62 (m, 2H).

### Example 82: 3-(5-(1-(3-(4-(6-(4-chloro-3-cyclopropyl-1H-pyrrolo[2,3-b]pyridin-5-yl)pyridin-2-yl)-3-oxopiperazin-1-yl) propyl)piperidin-4-yl)-1-oxoisoindolin-2-yl)piperidine-2,6-dione

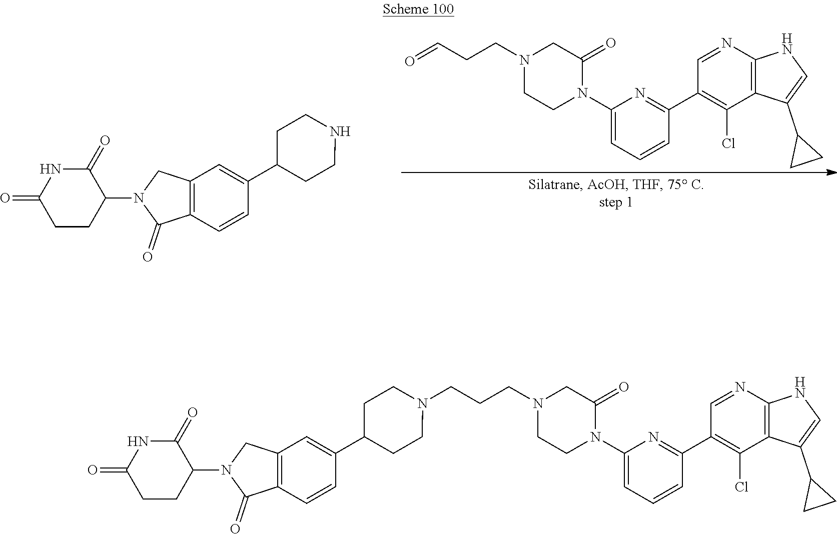 [SMILES: O=C1CCC(N2Cc3cc(C4CCNCC4)ccc3C2=O)C(=O)N1 ; O=CCCN1CCN(c2cccc(-c3cnc4[nH]cc(C5CC5)c4c3Cl)n2)C(=O)C1 ; O=C1CCC(N2Cc3cc(C4CCN(CCCN5CCN(c6cccc(-c7cnc8[nH]cc(C9CC9)c8c7Cl)n6)C(=O)C5)CC4)ccc3C2=O)C(=O)N1]

Step 1. Preparation of 3-(5-(1-(3-(4-(6-(4-chloro-3-cyclopropyl-1H-pyrrolo[2,3-b]pyridin-5-yl)pyridin-2-yl)-3-oxopiperazin-1-yl) propyl)piperidin-4-yl)-1-oxoisoindolin-2-yl)piperidine-2,6-dione: Following General step M2, the product was obtained as a white solid (55 mg, 12%). Mass (m/z): 753.8 [M+H]⁺. ¹H NMR (400 MHZ, DMSO-d₆) δ 11.82 (d, J=2.2 Hz, 1H), 10.99 (s, 1H), 8.32 (s, 1H), 7.94 (dd, J=5.8, 2.2 Hz, 2H), 7.65 (d, J=7.8 Hz, 1H), 7.59-7.53 (m, 1H), 7.49 (s, 1H), 7.40 (d, J=7.8 Hz, 1H), 7.32 (d, J=1.6 Hz, 1H), 5.10 (dd, J=13.2, 5.0 Hz, 1H), 4.42 (d, J=17.4 Hz, 1H), 4.29 (d, J=17.4 Hz, 1H), 4.00-3.94 (m, 2H), 3.16 (d, J=10.6 Hz, 2H), 2.99-2.77 (m, 4H), 2.64 (dd, J=39.6, 16.0 Hz, 4H), 2.48-2.18 (m, 7H), 1.99 (dd, J=10.2, 4.8 Hz, 1H), 1.89-1.68 (m, 6H), 0.86 (qd, J=6.0, 4.0 Hz, 2H), 0.68-0.63 (m, 2H).

### Example 83: 3-[5-(1-{3-[4-(6-{4-chloro-3-cyclopropyl-1H-pyrrolo[2,3-b]pyridin-3-yl}pyridin-2-yl)-3-oxopiperazin-1-yl]propyl}-3,6-dihydro-2H-pyridin-4-yl)-1-oxo-3H-isoindol-2-yl]piperidine-2,6-dione

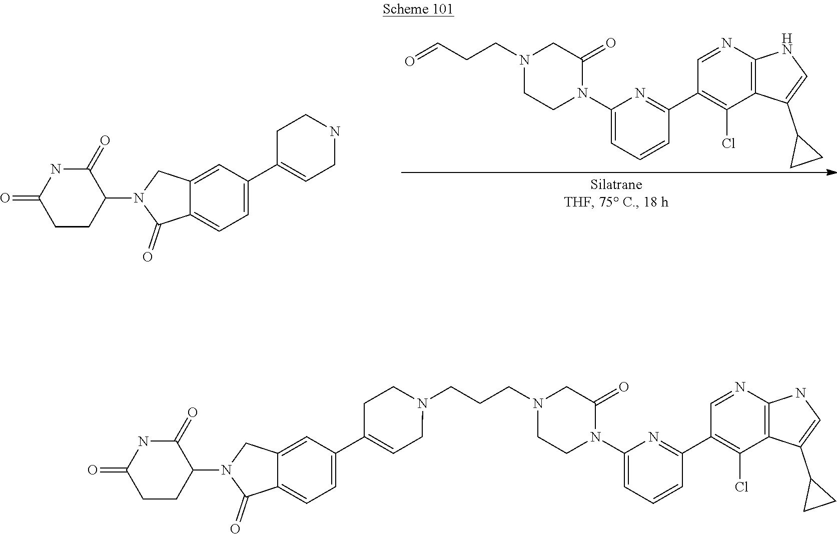 [SMILES: O=C1CCC(N2Cc3cc(C4=CCNCC4)ccc3C2=O)C(=O)N1 ; O=CCCN1CCN(c2cccc(-c3cnc4[nH]cc(C5CC5)c4c3Cl)n2)C(=O)C1 ; O=C1CCC(N2Cc3cc(C4=CCN(CCCN5CCN(c6cccc(-c7cnc8[nH]cc(C9CC9)c8c7Cl)n6)C(=O)C5)CC4)ccc3C2=O)C(=O)N1]

Step 1. Preparation of 3-[5-(1-{3-[4-(6-{4-chloro-3-cyclopropyl-1H-pyrrolo[2,3-b]pyridin-3-yl}pyridin-2-yl)-3-oxopiperazin-1-yl]propyl}-3,6-dihydro-2H-pyridin-4-yl)-1-oxo-3H-isoindol-2-yl]piperidine-2,6-dione: Following General step M2, the product (30 mg, 6.59%) was obtained as a white solid. Mass (m/z): 732.8 [M+H]⁺. ¹HNMR (400 MHZ, DMSO-d₆) δ 11.79 (d, J=2.3 Hz, 1H), 10.96 (s, 1H), 8.28 (s, 1H), 7.90 (m, 2H), 7.63 (m, 2H), 7.53 (m, 2H), 7.29 (d, J=2.0 Hz, 1H), 6.29 (s, 1H), 5.07 (dd, J=13.3, 5.1 Hz, 1H), 4.34 (dd, J=52.2, 17.4 Hz, 2H), 3.92 (m, 2H), 3.18 (s, 4H), 2.76 (dd, J=19.3, 13.4 Hz, 4H), 2.52 (s, 4H), 2.41 (m, 4H), 1.97 (s, 1H), 1.70 (m, 2H), 1.19 (s, 2H), 0.82 (m, 2H), 0.62 (m, 2H).

### Example 84: 5-(1-(3-(4-(6-(4-chloro-3-cyclopropyl-1H-pyrrolo[2,3-b]pyridin-5-yl)pyridin-2-yl)-3-oxopiperazin-1-yl) propyl)piperidin-4-yl)-2-(2,6-dioxopiperidin-3-yl)isoindoline-1,3-dione

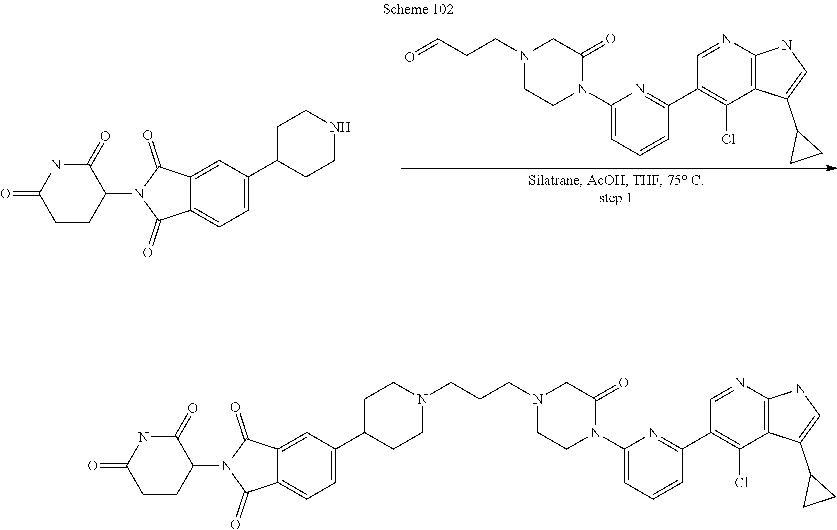 [SMILES: O=C1CCC(N2C(=O)c3ccc(C4CCNCC4)cc3C2=O)C(=O)N1 ; O=CCCN1CCN(c2cccc(-c3cnc4[nH]cc(C5CC5)c4c3Cl)n2)C(=O)C1 ; O=C1CCC(N2C(=O)c3ccc(C4CCN(CCCN5CCN(c6cccc(-c7cnc8[nH]cc(C9CC9)c8c7Cl)n6)C(=O)C5)CC4)cc3C2=O)C(=O)N1]

Step 1. Preparation of 5-(1-(3-(4-(6-(4-chloro-3-cyclopropyl-1H-pyrrolo[2,3-b]pyridin-5-yl)pyridin-2-yl)-3-oxopiperazin-1-yl) propyl)piperidin-4-yl)-2-(2,6-dioxopiperidin-3-yl)isoindoline-1,3-dione: Following General step M2, the product was obtained as a white solid (47 mg, 10%). Mass (m/z): 749.0 [M+H]⁺. ¹H NMR (400 MHZ, DMSO-d₆) δ 11.84 (s, 2H), 8.33 (s, 2H), 8.06-7.89 (m, 4H), 7.62 (d, J=7.4 Hz, 2H), 7.34 (d, J=1.8 Hz, 2H), 4.17 (s, 4H), 3.93 (s, 4H), 3.43 (s, 4H), 3.06 (s, 4), 2.28-2.16 (m, 2H), 2.03 (s, 2H), 0.88-0.84 (m, 4H), 0.68-0.64 (m, 4H).

### Example 85: 5-(1-(3-(4-(6-(4-chloro-3-cyclopropyl-1H-pyrrolo[2,3-b]pyridin-5-yl)pyridin-2-yl)-3-oxopiperazin-1-yl) propyl)-1,2,3,6-tetrahydropyridin-4-yl)-2-(2,6-dioxopiperidin-3-yl)isoindoline-1,3-dione

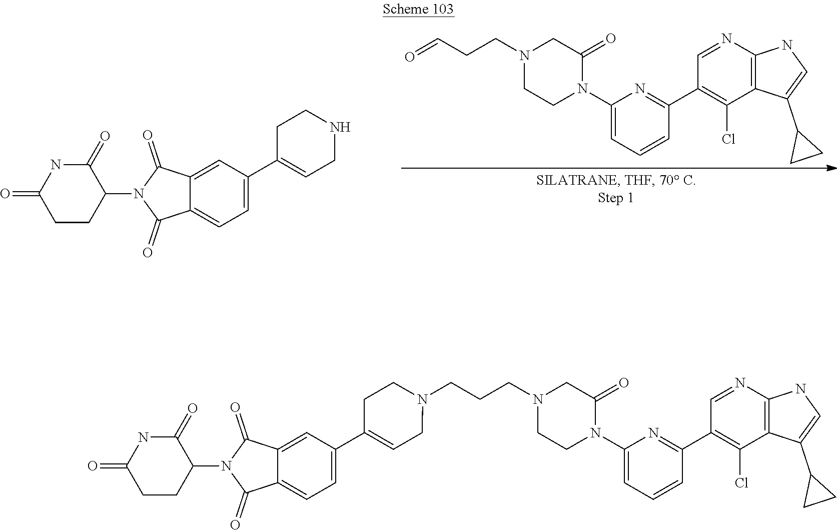 [SMILES: O=C1CCC(N2C(=O)c3ccc(C4=CCNCC4)cc3C2=O)C(=O)N1 ; O=CCCN1CCN(c2cccc(-c3cnc4[nH]cc(C5CC5)c4c3Cl)n2)C(=O)C1 ; O=C1CCC(N2C(=O)c3ccc(C4=CCN(CCCN5CCN(c6cccc(-c7cnc8[nH]cc(C9CC9)c8c7Cl)n6)C(=O)C5)CC4)cc3C2=O)C(=O)N1]

Step 1. Preparation of 5-(1-(3-(4-(6-(4-chloro-3-cyclopropyl-1H-pyrrolo[2,3-b]pyridin-5-yl)pyridin-2-yl)-3-oxopiperazin-1-yl) propyl)-1,2,3,6-tetrahydropyridin-4-yl)-2-(2,6-dioxopiperidin-3-yl)isoindoline-1,3-dione: Following General step M2, the product was obtained as a white solid (67 mg, 14%). Mass (m/z): 747.0 [M+H]⁺. ¹H NMR (400 MHZ, DMSO-d₆) δ 9.19 (s, 1H), 8.61 (s, 1H), 8.37 (s, 1H), 8.00 (d, J=8.1 Hz, 1H), 7.90-7.77 (m, 3H), 7.74 (d, J=7.6 Hz, 1H), 7.52 (d, J=7.4 Hz, 1H), 7.03 (s, 1H), 6.23 (s, 1H), 4.99 (dd, J=12.4, 5.2 Hz, 1H), 4.16-4.08 (m, 2H), 3.76-3.66 (m, 2H), 3.29-3.18 (m, 2H), 3.10-3.01 (m, 2H), 2.96-2.74 (m, 7H), 2.65-2.57 (m, 2H), 2.31-2.21 (m, 2H), 2.20-2.12 (m, 2H), 2.10-2.04 (m, 2H), 0.97-0.89 (m, 2H), 0.68-0.61 (m, 2H).

### Example 86: 3-cyclopropyl-1H-pyrrolo 3-(4-((1-(3-(4-(3-(4-chloro-3-cyclopropyl-1H-pyrrolo[2,3-b]pyridin-5-yl)phenyl)-3-oxopiperazin-1-yl) propyl)piperidin-4-yl)oxy)phenyl)piperidine-2,6-dione

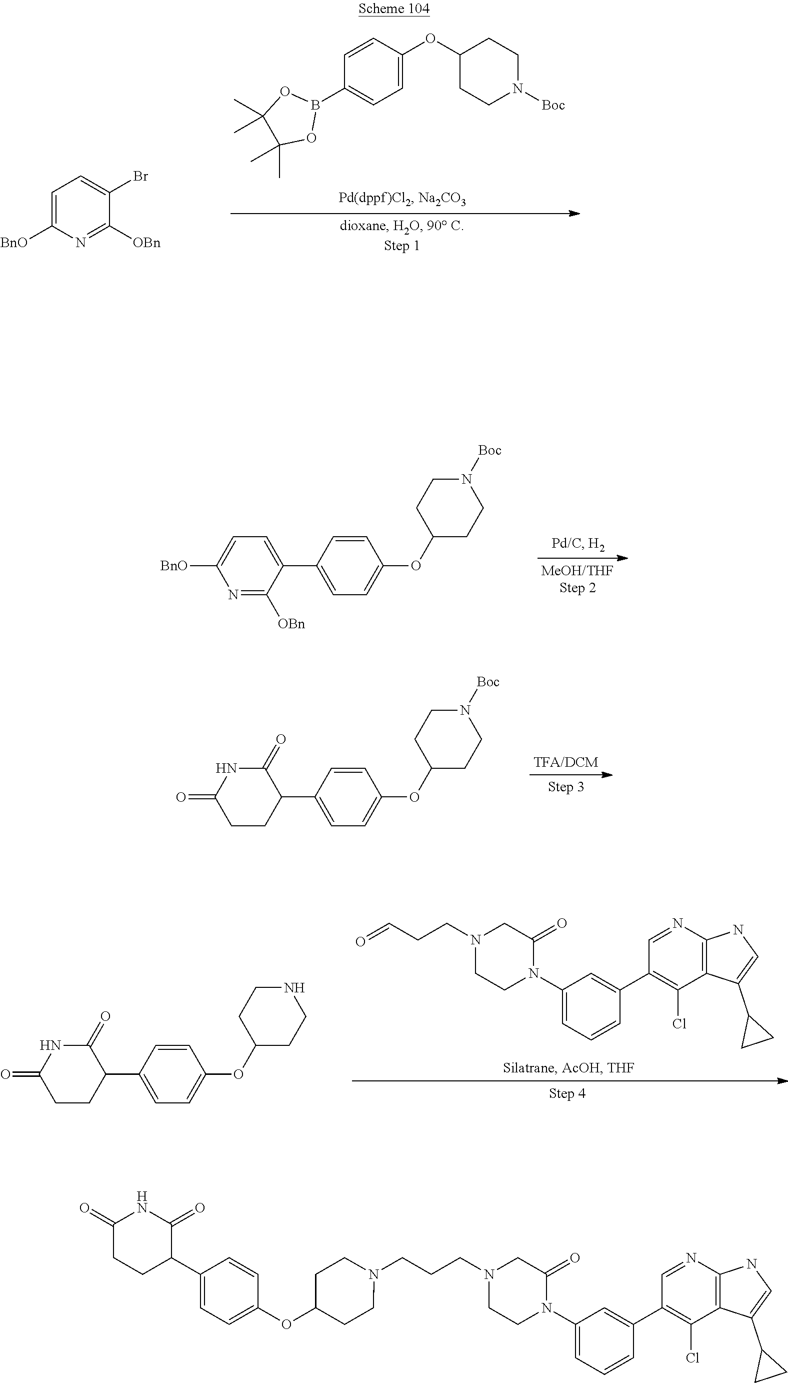

Step 1. Preparation of (4-{4-[2,6-bis(benzyloxy)pyridin-3-yl]phenoxy}piperidin-1-yl) tert-butyl formate: To a solution of 2,6-bis(benzyloxy)-3-bromopyridine (0.5 g, 1.35 mmol) and tert-butyl {4-[4-(4,4,5,5-tetramethyl-1,3,2-dioxaborolan-2-yl) phenoxy]piperidin-1-yl}formate (546 mg, 1.35 mmol) in H₂O (2 mL) and 1,4-dioxane (20 mL) was added Pd(dppf)Cl₂ (98 mg, 0.135 mmol) and Na₂CO₃ (429 mg, 4.05 mmol) at 25° C. The reaction mixture was stirred at 90° C. under N₂ for 6 hrs. Water (50 mL) was added and extracted with EA (30 mL×2). The organic layer was washed with brine (20 mL×2), then dried over anhydrous Na₂SO₄. After filtration, the solution was concentrated under vacuum. The residue was purified by flash chromatography (PE/EA=10:1) to give the product (4-{4-[2,6-bis(benzyloxy)pyridin-3-yl]phenoxy}piperidin-1-yl) tert-butyl formate (500 mg, 52%) as yellow oil. Mass (m/z): 566.9 [M+H]⁺.

Step 2. Preparation of tert-butyl {4-[4-(2,6-dioxopiperidin-3-yl) phenoxy]piperidin-1-yl}formate: To a solution of (4-{4-[2,6-bis(benzyloxy)pyridin-3-yl]phenoxy}piperidin-1-yl) tert-butyl formate (500 mg, 0.88 mmol) in MeOH (6 mL) and THF (6 mL) was added 10% Pd/C (52 mg, 20% wt/wt). The mixture was stirred under 0.4 MPa of H₂ at 40° C. for 16 hrs. The mixture was filtered and the filtrate was concentrated. The residue was purified by Combiflash (DCM/MeOH=20:1) to give the product as a white solid (260 mg, 72%). Mass (m/z): 410.9 [M+Na]⁺.

Step 3. Preparation of 3-[4-(piperidin-4-yloxy)phenyl]piperidine-2,6-dione: Following General step B2, the product was obtained as a light yellow solid (200 mg, 95%). Mass (m/z): 88.9 [M+H]⁺.

Step 4. Preparation of 3-{4-[(1-{3-[4-(3-{4-chloro-3-cyclopropyl-1H-pyrrolo[2,3-b]pyridin-3-yl}phenyl)-3-oxopiperazin-1-yl]propyl}piperidin-4-yl)oxy]phenyl}piperidine-2,6-dione: Following General step M2, the product (30 mg, 8%) was obtained as a light yellow solid. Mass (m/z): 695.3 [M+H]⁺. ¹H NMR (400 MHZ, DMSO-d₆) δ 11.78 (d, J=2.4 Hz, 1H), 10.81 (s, 1H), 8.13 (s, 1H), 7.56 (t, J=7.8 Hz, 1H), 7.49 (s, 1H), 7.43 (dd, J=13.2, 8.2 Hz, 2H), 7.32 (d, J=1.8 Hz, 1H), 7.16 (d, J=8.6 Hz, 2H), 6.97 (d, J=8.6 Hz, 2H), 4.66 (s, 1H), 3.93 (s, 2H), 3.79 (dd, J=11.6, 4.8 Hz, 5H), 3.22 (d, J=7.3 Hz, 4H), 3.03 (s, 2H), 2.65 (m, 2H), 2.10 (m, 10H), 1.23 (s, 1H), 0.85 (m, 2H), 0.65 (m, 2H).

### Example 87: 3-cyclopropyl-1H-pyrrolo3-(4-(1-(4-(4-(3-(4-chloro-3-cyclopropyl-1H-pyrrolo[2,3-b]pyridin-5-yl)phenyl)-3-oxopiperazin-1-yl)butyl)piperidin-4-yl)phenyl)piperidine-2,6-dione

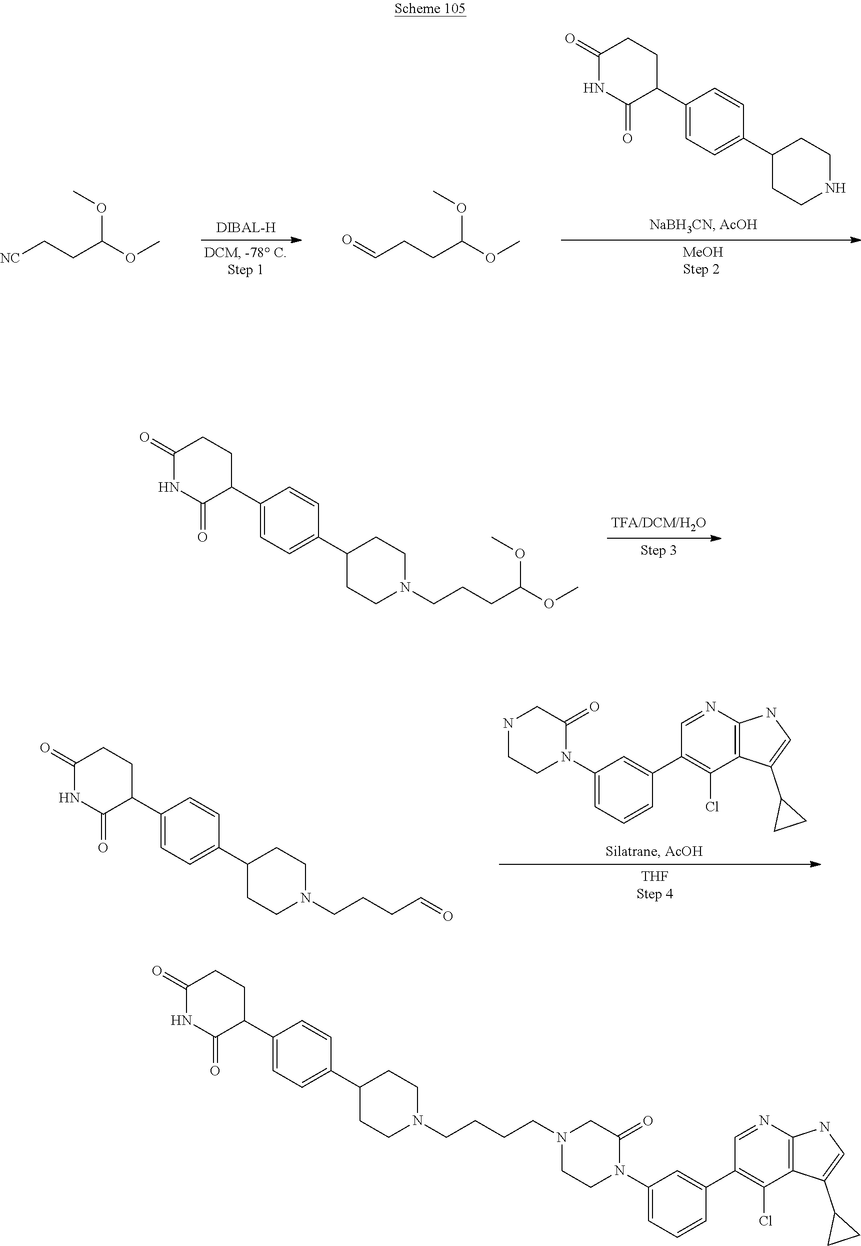

Step 1. Preparation of 4,4-dimethoxybutanal: To a solution of 4,4-dimethoxybutanenitrile (1.2 g, 9.3 mmol) in DCM (50 mL) at −78° C. under nitrogen was added DIBAL-H (1 M in hexane, 21.1 mL, 21.1 mmol). The solution was stirred at −78° C. under N₂ for 3 hrs. The mixture was slowly warmed to 25° C. and quenched with sat. NH₄Cl solution (50 mL). The mixture was extracted with DCM (30 mL×3). The combined organic layer was washed with brine (30 mL×3), then dried over anhydrous Na₂SO₄. After filtration, the solution was concentrated under vacuum to give the product 4,4-dimethoxybutanal (600 mg, 48%) as brown oil.

Step 2. Preparation of 3-{4-[1-(4,4-dimethoxybutyl)piperidin-4-yl]phenyl}piperidine-2,6-dione: To a solution of tert-butyl {4-[4-(2,6-dioxopiperidin-3-yl)phenyl]piperidin-1-yl}formate (100 mg, 0.27 mmol) in MeOH (10 mL) was added 4,4-dimethoxybutanal (247 mg, 1.88 mmol), NaBH₃CN (50 mg, 0.80 mmol) and AcOH (0.1 mL) at 0° C. The reaction mixture was stirred at 25° C. under N₂ for 16 hrs. The solvent was removed under reduced pressure and the residue was purified by Combiflash (DCM/MeOH=95:5) to give the product as yellow oil (150 mg, 86%). Mass (m/z): 389.0 [M+H]⁺.

Step 3. Preparation of 4-{4-[4-(2,6-dioxopiperidin-3-yl)phenyl]piperidin-1-yl}butanal: Following General step B2, the desired product (150 mg, 79%) was obtained as a brown oil. Mass (m/z): 343.2 [M+H]⁺.

Step 4. Preparation of 3-[4-(1-{4-[4-(3-{4-chloro-3-cyclopropyl-1H-pyrrolo[2,3-b]pyridin-3-yl}phenyl)-3-oxopiperazin-1-yl]butyl}piperidin-4-yl)phenyl]piperidine-2,6-dione: Following General step M2, the product (10 mg, 3.3%) was obtained as a white solid. Mass (m/z): 692.8 [M+H]⁺. ¹HNMR (400 MHZ, DMSO-d₆) δ 11.75 (d, J=1.8 Hz, 1H), 10.81 (s, 1H), 8.09 (s, 1H), 7.52 (d, J=7.8 Hz, 1H), 7.46 (s, 1H), 7.40 (dd, J=16.4, 7.8 Hz, 2H), 7.29 (d, J=1.6 Hz, 1H), 7.16 (s, 4H), 3.82 (m, 6H), 3.55 (s, 2H), 3.06 (d, J=29.2 Hz, 6H), 2.80 (s, 1H), 2.62 (m, 2H), 2.14 (dt, J=10.4, 6.0 Hz, 2H), 1.98 (dd, J=8.2, 3.8 Hz, 3H), 1.84 (d, J=11.4 Hz, 2H), 1.70 (s, 4H), 1.19 (s, 1H), 0.81 (dt, J=8.2, 2.6 Hz, 2H), 0.61 (q, J=5.4 Hz, 2H).

### Example 88: 3-cyclopropyl-1H-pyrrolo (1-(2-(2,6-dioxopiperidin-3-yl)-1,3-dioxoisoindolin-5-yl)piperidin-4-yl)methyl 4-(3-(4-chloro-3-cyclopropyl-1H-pyrrolo[2,3-b]pyridin-5-yl)phenyl)-3-oxopiperazine-1-carboxylate

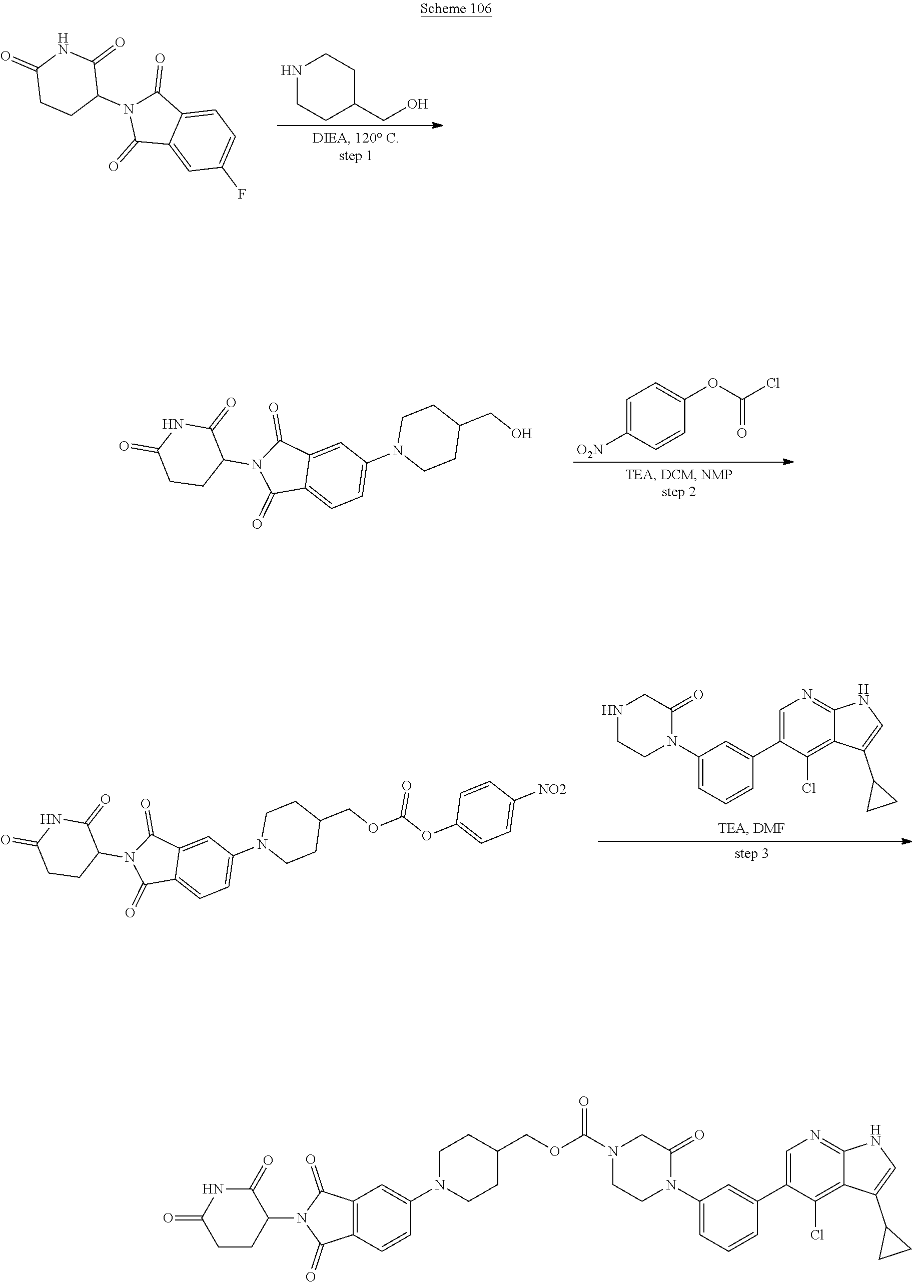

Step 1. Preparation of 2-(2,6-dioxopiperidin-3-yl)-5-[4-(hydroxymethyl)piperidin-1-yl]isoindole-1,3-dione: Following General step L, the product (1.2 g, 83%) was obtained as a yellow solid. Mass (m/z): 371.8 [M+H]⁺.

Step 2. (General Step J2) Preparation of {1-[2-(2,6-dioxopiperidin-3-yl)-1,3-dioxoisoindol-5-yl]piperidin-4-yl}methyl (4-nitrophenyl) carbonate: To a solution of 2-(2,6-dioxopiperidin-3-yl)-5-[4-(hydroxymethyl)piperidin-1-yl]isoindole-1,3-dione (600 mg, 1.62 mmol) and NMM (179 mg, 1.78 mmol) in DCM (10 mL) at 0° C. was added 4-nitrophenyl chloroformate (325 mg, 1.62 mmol). The reaction mixture was stirred at 25° C. under N₂ for 16 hrs. The solution was concentrated in vacuo and the residue was purified by Combiflash (PE/EA=1:1) to give the product (450 mg, 50%) as a yellow solid. Mass (m/z): 536.9 [M+H]⁺.

Step 3. (General Step G3) Preparation of {1-[2-(2,6-dioxopiperidin-3-yl)-1,3-dioxoisoindol-5-yl]piperidin-4-yl}methyl 4-(3-{4-chloro-3-cyclopropyl-1H-pyrrolo[2,3-b]pyridin-3-yl}phenyl)-3-oxopiperazine-1-carboxylate: To a solution of {1-[2-(2,6-dioxopiperidin-3-yl)-1,3-dioxoisoindol-5-yl]piperidin-4-yl}methyl (4-nitrophenyl) carbonate (70 mg, 0.13 mmol) in DMF (6 mL) was added 1-(3-{4-chloro-3-cyclopropyl-1H-pyrrolo[2,3-b]pyridin-3-yl}phenyl)piperazin-2-one (47 mg, 0.13 mmol) and TEA (26.41 mg, 0.26 mmol) at 25° C. The reaction mixture was stirred at 25° C. under N₂ for 16 hrs. After the reaction completed, H₂O (30 mL) was added to the reaction mixture, and then extracted with EA (20 mL×3). The combined organic layer was washed with brine (50 mL×2), then dried over anhydrous Na₂SO₄. After filtration, the solution was concentrated under vacuum, and the residue was purified by flash Chromatography (DCM/MeOH=97:3) to give the product as a yellow solid (60 mg, 57%). Mass (m/z): 764.3 [M+H]⁺. ¹H NMR (400 MHZ, DMSO-d₆) δ 11.76 (d, J=2.0 Hz, 1H), 11.08 (s, 1H), 8.12 (s, 1H), 7.65 (d, J=8.4 Hz, 1H), 7.56 (m, 2H), 7.49 (d, J=3.4 Hz, 1H), 7.40 (d, J=7.6 Hz, 2H), 7.31 (m, 2H), 7.24 (d, J=8.6 Hz, 1H), 5.06 (dd, J=12.8, 5.4 Hz, 1H), 4.11 (m, 4H), 3.96 (d, J=6.4 Hz, 2H), 3.82 (d, J=5.2 Hz, 2H), 3.75 (s, 2H), 2.98 (t, J=11.8 Hz, 2H), 2.20 (m, 1H), 1.99 (d, J=5.2 Hz, 2H), 1.78 (d, J=11.0 Hz, 2H), 1.24 (d, J=9.8 Hz, 4H), 0.85 (m, 2H), 0.65 (t, J=4.6 Hz, 2H).

### Example 89: 3-ethyl-1H-pyrrolo 5-(3-((4-(3-(4-chloro-3-ethyl-1H-pyrrolo[2,3-b]pyridin-5-yl)phenyl)-3-oxopiperazin-1-yl)methyl) azetidin-1-yl)-2-(2,6-dioxopiperidin-3-yl)isoindoline-1,3-dione

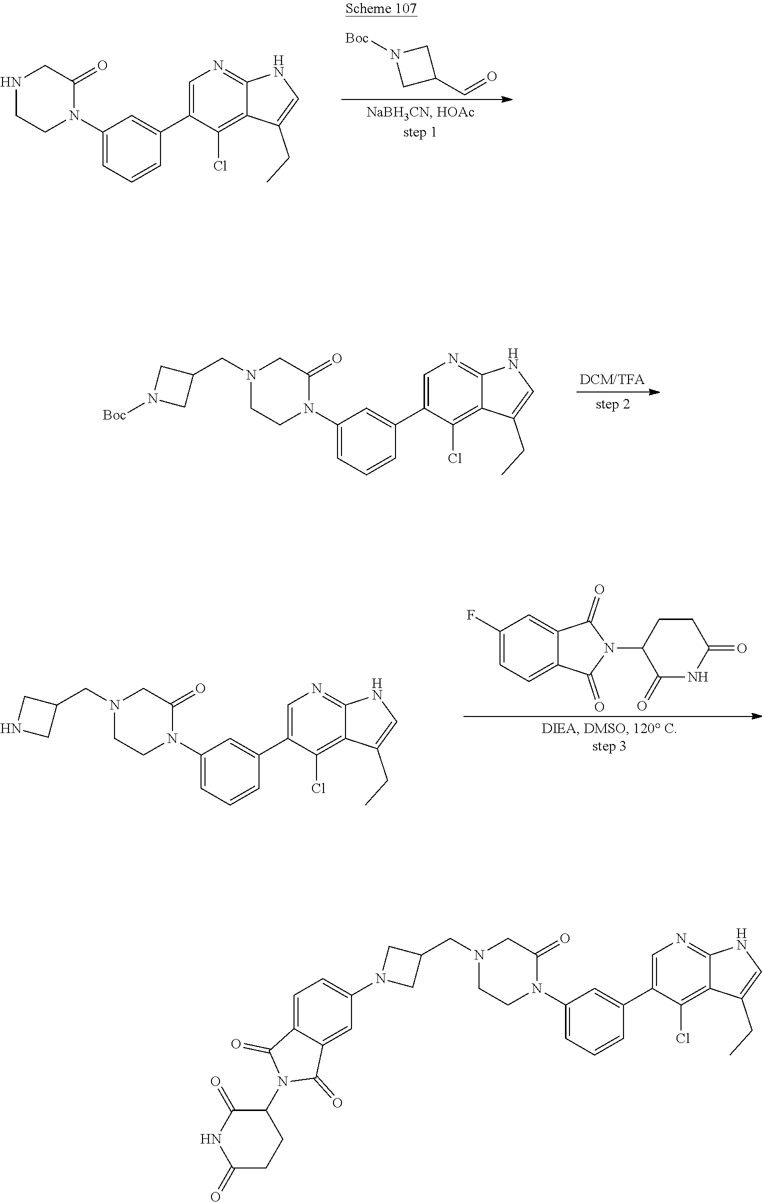 [SMILES: CCc1c[nH]c2ncc(-c3cccc(N4CCNCC4=O)c3)c(Cl)c12 ; CC(C)(C)OC(=O)N1CC(C=O)C1 ; CCc1c[nH]c2ncc(-c3cccc(N4CCN(CC5CN(C(=O)OC(C)(C)C)C5)CC4=O)c3)c(Cl)c12 ; CCc1c[nH]c2ncc(-c3cccc(N4CCN(CC5CNC5)CC4=O)c3)c(Cl)c12 ; O=C1CCC(N2C(=O)c3ccc(F)cc3C2=O)C(=O)N1 ; CCc1c[nH]c2ncc(-c3cccc(N4CCN(CC5CN(c6ccc7c(c6)C(=O)N(C6CCC(=O)NC6=O)C7=O)C5)CC4=O)c3)c(Cl)c12]

Step 1. Preparation of tert-butyl (3-{[4-(3-{4-chloro-3-ethyl-1H-pyrrolo[2,3-b]pyridin-3-yl}phenyl)-3-oxopiperazin-1-yl]methyl}azetidin-1-yl) formate: From 1-(3-{4-chloro-3-ethyl-1H-pyrrolo[2,3-b]pyridin-3-yl}phenyl)piperazin-2-one (200 mg, 0.564 mmol) and tert-butyl (3-formylazetidin-1-yl) formate (105 mg, 0.564 mmol), following General step M1, the product tert-butyl (3-{[4-(3-{4-chloro-3-ethyl-1H-pyrrolo[2,3-b]pyridin-3-yl}phenyl)-3-oxopiperazin-1-yl]methyl}azetidin-1-yl) formate (230 mg, 74%) was obtained as a yellow solid. Mass (m/z): 424.2 [M+H]⁺.

Step 2. Preparation of 4-(azetidin-3-ylmethyl)-1-(3-{4-chloro-3-ethyl-1H-pyrrolo[2,3-b]pyridin-3-yl}phenyl)piperazin-2-one: From tert-butyl (3-{[4-(3-{4-chloro-3-ethyl-1H-pyrrolo[2,3-b]pyridin-3-yl}phenyl)-3-oxopiperazin-1-yl]methyl}azetidin-1-yl) formate (230 mg, 0.438 mmol), following General step B2, the product 4-(azetidin-3-ylmethyl)-1-(3-{4-chloro-3-ethyl-1H-pyrrolo[2,3-b]pyridin-3-yl}phenyl)piperazin-2-one (220 mg, 95%) was obtained as a yellow solid. Mass (m/z): 424.1 [M+H]⁺.

Step 3. Preparation of 5-(3-{[4-(3-{4-chloro-3-ethyl-1H-pyrrolo[2,3-b]pyridin-3-yl}phenyl)-3-oxopiperazin-1-yl]methyl}azetidin-1-yl)-2-(2,6-dioxopiperidin-3-yl) isoindole-1,3-dione: From 4-(azetidin-3-ylmethyl)-1-(3-{4-chloro-3-ethyl-1H-pyrrolo[2,3-b]pyridin-3-yl}phenyl)piperazin-2-one (100 mg, 0.236 mmol) and 2-(2,6-dioxopiperidin-3-yl)-5-fluoroisoindole-1,3-dione (326 mg, 1.18 mmol), following General step L, the product 5-(3-{[4-(3-{4-chloro-3-ethyl-1H-pyrrolo[2,3-b]pyridin-3-yl}phenyl)-3-oxopiperazin-1-yl]methyl}azetidin-1-yl)-2-(2,6-dioxopiperidin-3-yl) isoindole-1,3-dione (65 mg, 39%) was obtained as a yellow solid. Mass (m/z): 680.0 [M+H]⁺. ¹H NMR (400 MHZ, DMSO-d₆) δ 11.80 (d, J=2.2 Hz, 1H), 11.08 (s, 1H), 8.13 (s, 1H), 7.64 (d, J=8.2 Hz, 1H), 7.50 (dd, J=16.2, 8.4 Hz, 2H), 7.40 (t, J=8.8 Hz, 3H), 6.79 (d, J=1.8 Hz, 1H), 6.66 (dd, J=8.4, 1.8 Hz, 1H), 5.05 (dd, J=12.8, 5.2 Hz, 1H), 4.16 (t, J=8.2 Hz, 2H), 3.75 (s, 4H), 3.25 (s, 2H), 3.08 (s, 1H), 2.92 (dd, J=14.8, 7.4 Hz, 2H), 2.86 (d, J=8.2 Hz, 2H), 2.77 (s, 2H), 2.60-2.52 (m, 3H), 2.02-1.99 (m, 1H), 1.28 (t, J=7.4 Hz, 3H).

### Example 90: 5-((4-(3-(4-chloro-3-ethyl-1H-pyrrolo[2,3-b]pyridin-5-yl)phenyl)-3-oxopiperazin-1-yl)methyl)-2-(2,6-dioxopiperidin-3-yl)isoindoline-1,3-dione

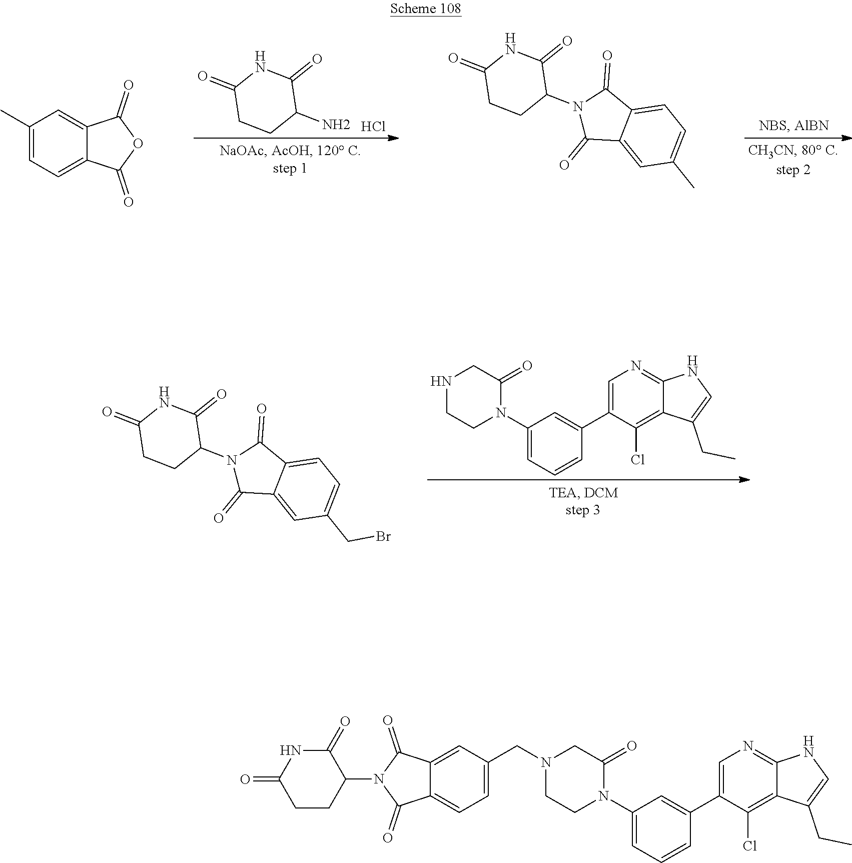

Step 1. Preparation of 2-(2,6-dioxopiperidin-3-yl)-5-methylisoindoline-1,3-dione: To a solution of 5-methyl-2-benzofuran-1,3-dione (3000 mg, 18.50 mmol) and 3-aminopiperidine-2,6-dione hydrochloride (3045 mg, 18.50 mmol) in AcOH (30 mL) was added NaOAc (3036 mg, 37.0 mmol). The reaction mixture was stirred at 120° C. under N₂ for 16 hrs. The reaction mixture was washed with water (200 mL) and filtered. The filter cake was freeze dried to give the product as a yellow solid (4200 mg, 66%). Mass (m/z): 273.0 [M+H]⁺.

Step 2. Preparation of 5-(bromomethyl)-2-(2,6-dioxopiperidin-3-yl)isoindoline-1,3-dione: To a solution of 2-(2,6-dioxopiperidin-3-yl)-5-methylisoindole-1,3-dione (4000 mg, 14.69 mmol) in acetonitrile (80 mL) was added N-Bromosuccinimide (3922 mg, 22.04 mmol) and AIBN (965 mg, 5.88 mmol). The reaction mixture was stirred at 80° C. under N₂ for 16 hrs. The reaction mixture was concentrated and purified by silica gel column chromatography (DCM/MeOH=20:1) to give the product as a yellow solid (5500 mg, 74%). Mass (m/z): 350.9 352.8 [M+H]⁺.

Step 3. Preparation of 5-((4-(3-(4-chloro-3-ethyl-1H-pyrrolo[2,3-b]pyridin-5-yl)phenyl)-3-oxopiperazin-1-yl)methyl)-2-(2,6-dioxopiperidin-3-yl)isoindoline-1,3-dione: To a solution of 5-(bromomethyl)-2-(2,6-dioxopiperidin-3-yl) isoindole-1,3-dione (100 mg, 0.28 mmol) and 1-(3-{4-chloro-3-ethyl-1H-pyrrolo[2,3-b]pyridin-3-yl}phenyl)piperazin-2-one (101 mg, 0.28 mmol) in DCM (3 mL) was added TEA (86 mg, 0.8544 mmol). The reaction mixture was stirred at 25° C. under N₂ for 16 hrs. The reaction mixture was concentrated and purified by Pre-HPLC (chromatographic column:—Gemini-C₁₈ 150×21.2 mm, 5 um, mobile phase: ACN-H2O (0.1% FA), gradient: 20-50) to give the product as a white solid (60 mg, 33%). Mass (m/z): 625.2 [M+H]⁺. ¹H NMR (400 MHZ, DMSO-d₆) δ 11.80 (d, J=2.2 Hz, 1H), 11.15 (s, 1H), 8.12 (s, 1H), 7.94 (d, J=6.6 Hz, 3H), 7.49 (dd, J=11.8, 4.7 Hz, 2H), 7.41 (d, J=9.3 Hz, 3H), 5.17 (dd, J=12.9, 5.3 Hz, 1H), 3.79 (s, 4H), 3.36-3.36 (m, 2H), 3.13-2.81 (m, 5H), 2.53 (s, 2H), 2.11-2.02 (m, 1H), 1.27 (t, J=7.4 Hz, 3H).

### Example 91: 3-(2,2-difluoroethyl)-1H-pyrrolo 2-(1-(2-(2,6-dioxopiperidin-3-yl)-1,3-dioxoisoindolin-5-yl) azetidin-3-yl)ethyl 4-(3-(4-chloro-3-(2,2-difluoroethyl)-1H-pyrrolo[2,3-b]pyridin-5-yl)phenyl)-3-oxopiperazine-1-carboxylate

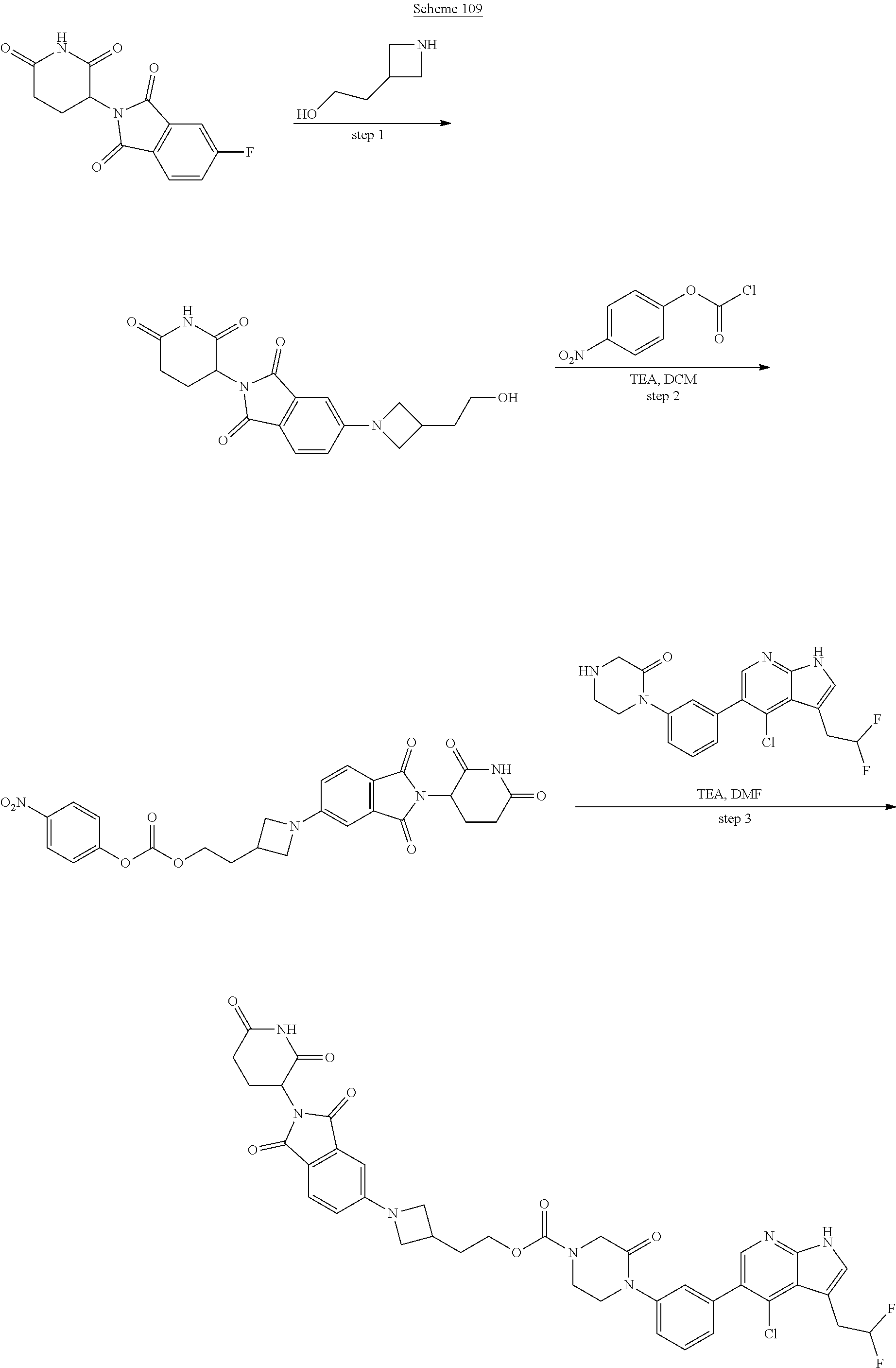

Step 1. Preparation of 2-(2,6-dioxopiperidin-3-yl)-5-[3-(2-hydroxyethyl) azetidin-1-yl]isoindole-1,3-dione: Following General step L, the product was obtained as a yellow solid (100 mg, 77.29%). Mass (m/z): 358.2 [M+H]⁺.

Step 2. Preparation of (2-{1-[2-(2,6-dioxopiperidin-3-yl)-1,3-dioxoisoindol-5-yl]azetidin-3-yl}ethyl)(4-nitrophenyl) carbonate: Following General step J2, the product (98 mg, 62%) was obtained as a yellow solid. Mass (m/z): 523.1 [M+H]⁺.

Step 3. Preparation of 2-{1-[2-(2,6-dioxopiperidin-3-yl)-1,3-dioxoisoindol-5-yl]azetidin-3-yl}ethyl 4-{3-[4-chloro-3-(2,2-difluoroethyl)-1H-pyrrolo[2,3-b]pyridin-3-yl]phenyl}-3-oxopiperazine-1-carboxylate: Following General step G3, the product was obtained as a yellow solid (20 mg, 13%). Mass (m/z): 774.3 [M+H]⁺. ¹H NMR (400 MHZ, DMSO-d₆) δ 12.12 (d, J=2.2 Hz, 1H), 11.07 (s, 1H), 8.18 (s, 1H), 7.60 (dd, J=12.8, 5.2 Hz, 2H), 7.53 (t, J=7.8 Hz, 1H), 7.49 (s, 1H), 7.41 (dd, J=14.0, 8.2 Hz, 2H), 6.77 (d, J=1.7 Hz, 1H), 6.62 (d, J=7.2 Hz, 1H), 6.29 (t, J=4.6 Hz, 1H), 5.05 (dd, J=12.8, 5.4 Hz, 1H), 4.14 (dt, J=12.2, 7.2 Hz, 6H), 3.84 (s, 2H), 3.73 (m, 4H), 3.52 (td, J=17.2, 4.4 Hz, 2H), 2.85 (m, 2H), 1.99 (d, J=6.8 Hz, 3H), 1.23 (s, 2H).

### Example 92: 3-(6-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)-1-methyl-1H-indazol-3-yl)piperidine-2,6-dione

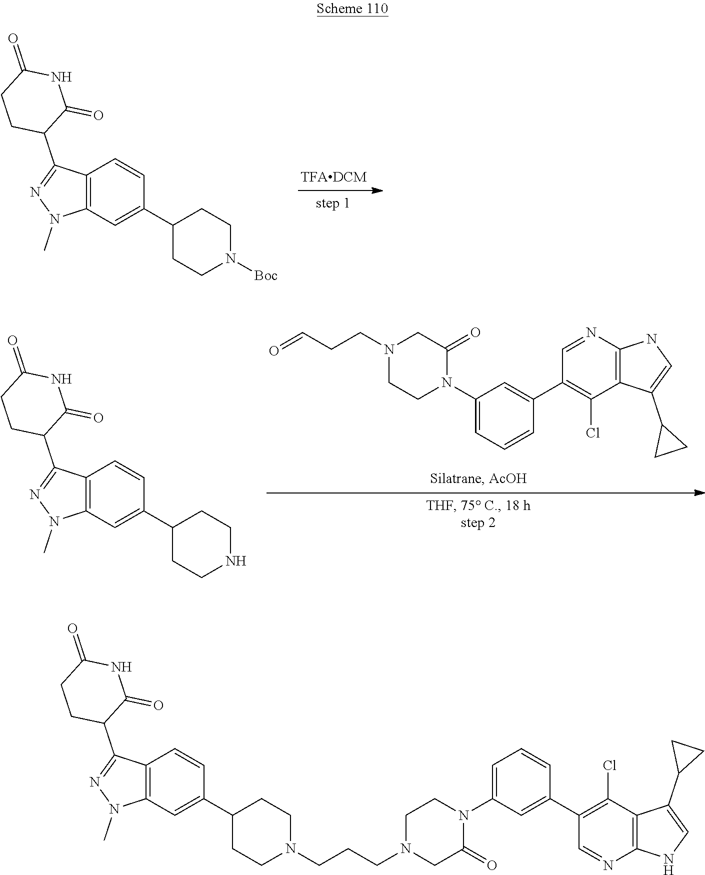 [SMILES: Cn1nc(C2CCC(=O)NC2=O)c2ccc(C3CCN(C(=O)OC(C)(C)C)CC3)cc21 ; Cn1nc(C2CCC(=O)NC2=O)c2ccc(C3CCNCC3)cc21 ; O=CCCN1CCN(c2cccc(-c3cnc4[nH]cc(C5CC5)c4c3Cl)c2)C(=O)C1 ; Cn1nc(C2CCC(=O)NC2=O)c2ccc(C3CCN(CCCN4CCN(c5cccc(-c6cnc7[nH]cc(C8CC8)c7c6Cl)c5)C(=O)C4)CC3)cc21]

Step 1. Preparation of 3-(1-methyl-6-(piperidin-4-yl)-1H-indazol-3-yl)piperidine-2,6-dione: Following General step B2, the desired product was obtained by suction filtration (63 mg, 94%) as a brown solid. Mass (m/z): 327 [M+H]⁺.

Step 2. Preparation of 3-(6-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl)-1-methyl-1H-indazol-3-yl) piperidine-2,6-dione: Following General step M2, the desired product (27 mg, 18%) was obtained as a white solid. Mass (m/z): 733.1 [M+H]⁺. ¹H NMR (400 MHZ, DMSO-d₆) δ 11.76 (s, 1H), 10.89 (s, 1H), 8.13 (d, J=5.4 Hz, 1H), 7.63 (d, J=8.4 Hz, 1H), 7.55-7.47 (m, 2H), 7.41 (dd, J=15.0, 6.6 Hz, 3H), 7.31 (s, 1H), 7.04 (d, J=8.2 Hz, 1H), 4.33 (dd, J=9.4, 5.0 Hz, 2H), 3.97 (s, 3H), 3.76 (s, 2H), 3.23 (s, 2H), 2.97 (d, J=13.4 Hz, 2H), 2.83 (s, 2H), 2.67 (s, 3H), 2.33 (s, 3H), 2.24-2.09 (m, 3H), 1.84 (dd, J=30.2, 14.4 Hz, 6H), 1.24 (s, 2H), 0.85 (d, J=8.2 Hz, 2H), 0.65 (d, J=3.6 Hz, 2H).

### Example 93: 1-(7-(1-(3-(4-(3-(4-chloro-3-cyclopropyl-1H-pyrrolo[2,3-b]pyridin-5-yl)phenyl)-3-oxopiperazin-1-yl) propyl)-1,2,3,6-tetrahydropyridin-4-yl) imidazo[1,2-a]pyridin-3-yl)dihydropyrimidine-2,4 (1H,3H)-dione

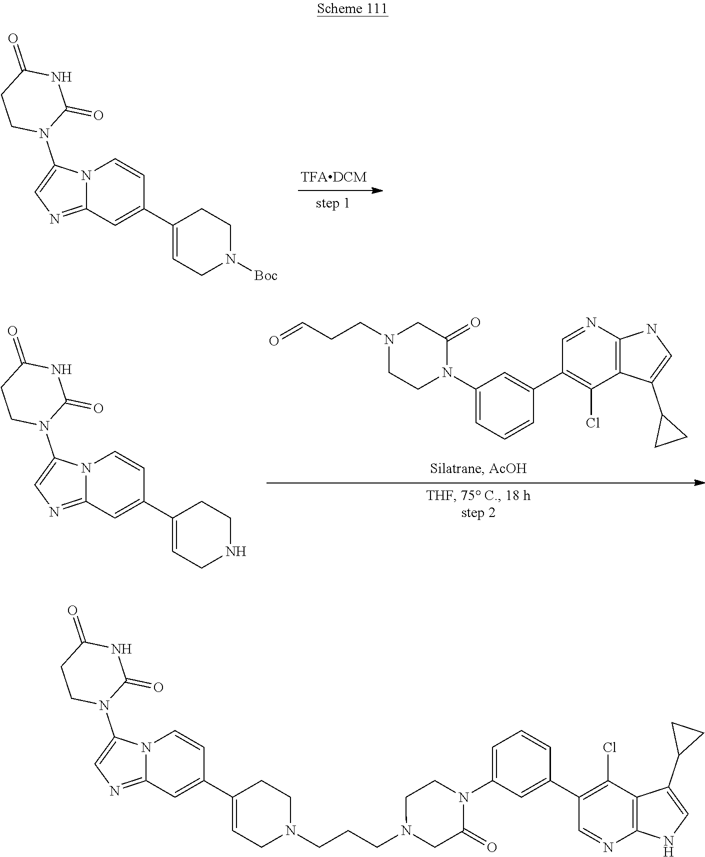 [SMILES: CC(C)(C)OC(=O)N1CC=C(c2ccn3c(N4CCC(=O)NC4=O)cnc3c2)CC1 ; O=C1CCN(c2cnc3cc(C4=CCNCC4)ccn23)C(=O)N1 ; O=CCCN1CCN(c2cccc(-c3cnc4[nH]cc(C5CC5)c4c3Cl)c2)C(=O)C1 ; O=C1CCN(c2cnc3cc(C4=CCN(CCCN5CCN(c6cccc(-c7cnc8[nH]cc(C9CC9)c8c7Cl)c6)C(=O)C5)CC4)ccn23)C(=O)N1]

Step 1. Preparation of 1-(7-(1,2,3,6-tetrahydropyridin-4-yl) imidazo[1,2-a]pyridin-3-yl)dihydro pyrimidine-2,4 (1H,3H)-dione: Following General step B2, the desired product was obtained by suction filtration (125 mg, 95%) as a brown solid. Mass (m/z): 312 [M+H]⁺.

Step 2. Preparation of 1-(7-(1-(3-(4-(3-(4-chloro-3-cyclopropyl-1H-pyrrolo[2,3-b]pyridin-5-yl)phenyl)-3-oxopiperazin-1-yl) propyl)-1,2,3,6-tetrahydropyridin-4-yl) imidazo[1,2-a]pyridin-3-yl)dihydropyrimidine-2,4 (1H,3H)-dione: Following General step M2, the desired product (15 mg, 4%) was obtained as a yellow solid. Mass (m/z): 718.1 [M+H]⁺. ¹H NMR (400 MHz, DMSO-d₆) δ11.76 (s, 1H), 10.66 (s, 1H), 8.21 (d, J=7.2 Hz, 1H), 8.13 (s, 1H), 7.58-7.45 (m, 4H), 7.40 (t, J=9.2 Hz, 2H), 7.31 (d, J=1.6 Hz, 1H), 7.19 (d, J=7.4 Hz, 1H), 6.45 (s, 1H), 3.77 (dt, J=10.2, 6.0 Hz, 4H), 3.19 (d, J=12.2 Hz, 5H), 2.82 (d, J=5.4 Hz, 4H), 2.75-2.64 (m, 3H), 2.61-2.53 (m, 3H), 2.33 (d, J=1.8 Hz, 1H), 2.21 (s, 1H), 1.73 (s, 2H), 0.89-0.79 (m, 2H), 0.68-0.60 (m, 2H).

### Example 94: 1-((2-(2,6-dioxopiperidin-3-yl)-1,3-dioxoisoindolin-5-yl)methyl)piperidin-4-yl 4-(3-(4-chloro-3-cyclopropyl-1H-pyrrolo[2,3-b]pyridin-5-yl)phenyl)-3-oxopiperazine-1-carboxylate

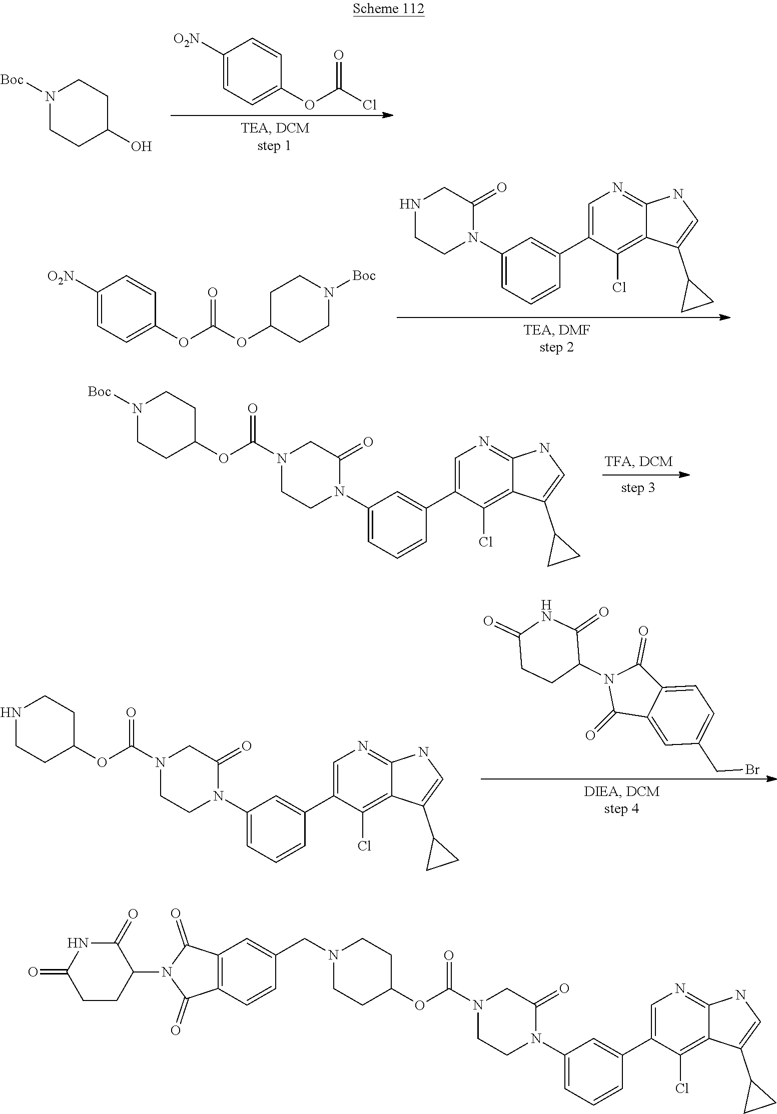

Step 1. Preparation of tert-butyl 4-(((4-nitrophenoxy) carbonyl)oxy)piperidine-1-carboxylate: Following General step J2, the product was obtained as a yellow solid (2000 mg, 88%). Mass (m/z): 389.0 [M+Na]⁺.

Step 2. Preparation of 1-(tert-butoxycarbonyl)piperidin-4-yl 4-(3-(4-chloro-3-cyclopropyl-1H-pyrrolo[2,3-b]pyridin-5-yl)phenyl)-3-oxopiperazine-1-carboxylate: Following General step G3, the product was obtained as a yellow oil (300 mg, 83%). Mass (m/z): 594.2 [M+H]⁺.

Step 3. Preparation of piperidin-4-yl 4-(3-(4-chloro-3-cyclopropyl-1H-pyrrolo[2,3-b]pyridin-5-yl)phenyl)-3-oxopiperazine-1-carboxylate: Following General step B2, the product was obtained as a yellow oil (700 mg, 98%). Mass (m/z): 494.2 [M+H]⁺.

Step 4. Preparation of 1-((2-(2,6-dioxopiperidin-3-yl)-1,3-dioxoisoindolin-5-yl)methyl)piperidin-4-yl 4-(3-(4-chloro-3-cyclopropyl-1H-pyrrolo[2,3-b]pyridin-5-yl)phenyl)-3-oxopiperazine-1-carboxylate: To a solution of piperidin-4-yl 4-(3-{4-chloro-3-cyclopropyl-1H-pyrrolo[2,3-b]pyridin-3-yl}phenyl)-3-oxopiperazine-1-carboxylate (300 mg, 0.61 mmol) and 5-(bromomethyl)-2-(2,6-dioxopiperidin-3-yl) isoindole-1,3-dione (213 mg, 0.61 mmol) in DCM (5 mL) was added DIEA (785 mg, 6.07 mmol). The reaction mixture was stirred at 25° C. under N₂ for 16 hrs. The reaction mixture was concentrated and purified by Prep-HPLC (chromatography column:—Gemini-C₁₈ 150×21.2 mm, 5 um, mobile phase: ACN-H2O (0.1% FA), gradient: 15-50) to give the product as a white solid (80 mg, 16%). Mass (m/z): 764.0 [M+H]⁺. ¹H NMR (400 MHZ, DMSO-d₆) δ 11.76 (d, J=2.2 Hz, 1H), 11.13 (s, 1H), 8.13 (d, J=5.1 Hz, 1H), 7.87 (dd, J=16.5, 11.2 Hz, 3H), 7.55-7.48 (m, 2H), 7.45-7.37 (m, 2H), 7.31 (d, J=1.7 Hz, 1H), 5.15 (dd, J=12.9, 5.4 Hz, 1H), 4.74-4.64 (m, 1H), 4.22-4.10 (m, 2H), 3.89-3.65 (m, 6H), 2.94-2.84 (m, 1H), 2.74-2.54 (m, 4H), 2.44-2.28 (m, 2H), 2.23-2.17 (m, 1H), 2.10-2.03 (m, 1H), 1.93-1.83 (m, 2H), 1.77-1.59 (m, 2H), 0.88-0.83 (m, 2H), 0.68-0.61 (m, 2H).

### Example 95: 5-(2-(1-(4-(3-(4-chloro-3-cyclopropyl-1H-pyrrolo[2,3-b]pyridin-5-yl)phenyl)-3-oxopiperazine-1-carbonyl) azetidin-3-yl)ethoxy)-2-(2,6-dioxopiperidin-3-yl)isoindoline-1,3-dione

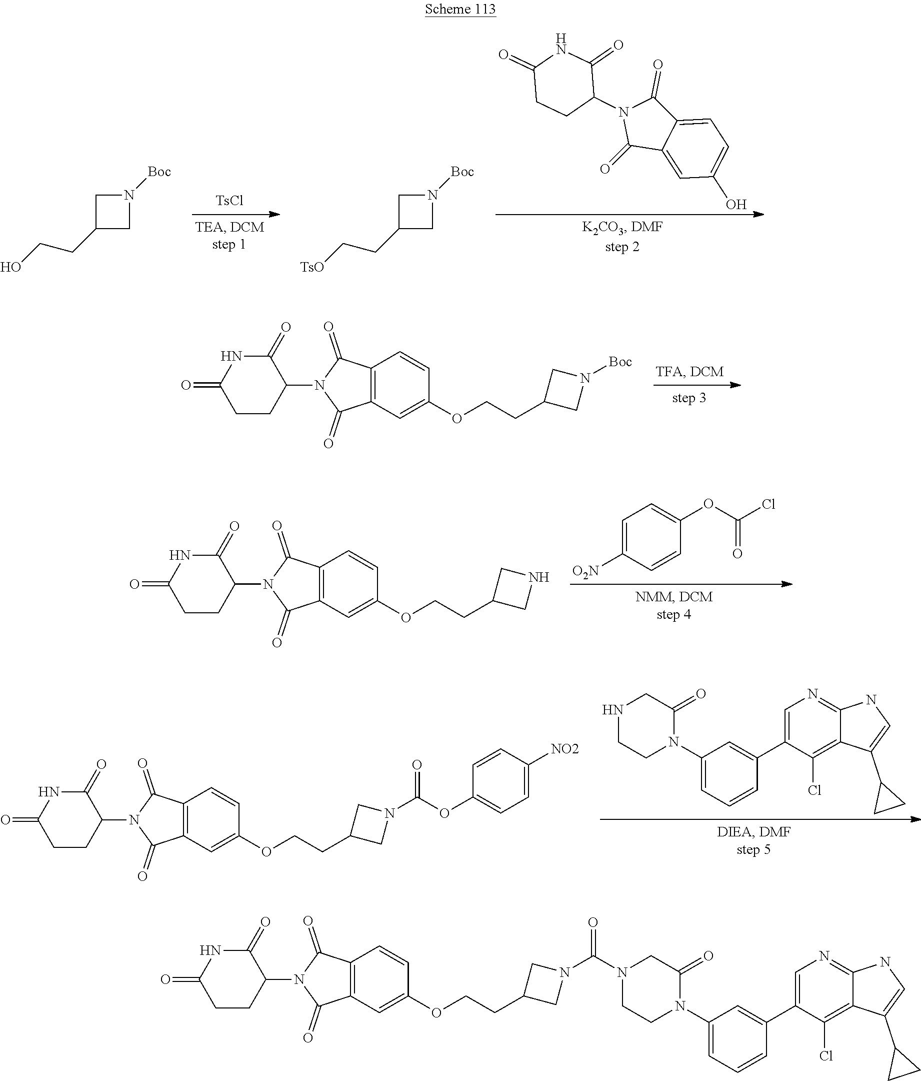

Step 1. Preparation of tert-butyl 3-(2-(tosyloxy)ethyl) azetidine-1-carboxylate: Following General step D, the product was obtained as colorless oil (1.6 g, 81%). Mass (m/z): 300.0 [M−55]⁺.

Step 2. Preparation of tert-butyl 3-(2-((2-(2,6-dioxopiperidin-3-yl)-1,3-dioxoisoindolin-5-yl)oxy)ethyl) azetidine-1-carboxylate: Following General step E, the product was obtained as yellow oil (1.8 g, 78%). Mass (m/z): 402.0 [M−55]⁺.

Step 3. Preparation of 5-(2-(azetidin-3-yl)ethoxy)-2-(2,6-dioxopiperidin-3-yl) isoindoline-1,3-dione: Following General step B2, the product was obtained as yellow oil (1 g, 96%). Mass (m/z): 358.1 [M+H]⁺.

Step 4. Preparation of 4-nitrophenyl 3-(2-((2-(2,6-dioxopiperidin-3-yl)-1,3-dioxoisoindolin-5-yl)oxy)ethyl) azetidine-1-carboxylate: To a solution of 5-[2-(azetidin-3-yl)ethoxy]-2-(2,6-dioxopiperidin-3-yl) isoindole-1,3-dione (700 mg, 1.95 mmol) and NMM (594 mg, 5.87 mmol) in DCM (10 mL) was added 4-nitrophenyl chloroformate (395 mg, 1.95 mmol). The reaction mixture was stirred at 25° C. for 16 hrs. The reaction mixture was concentrated and purified by silica gel column chromatography (PE/EA=1:1) to give the product as a yellow solid (600 mg, 29%). Mass (m/z): 523.0 [M+H]⁺.

Step 5. Preparation of 5-(2-(1-(4-(3-(4-chloro-3-cyclopropyl-1H-pyrrolo[2,3-b]pyridin-5-yl)phenyl)-3-oxopiperazine-1-carbonyl) azetidin-3-yl)ethoxy)-2-(2,6-dioxopiperidin-3-yl)isoindoline-1,3-dione: Following General step G3, the product was obtained as a white solid (50 mg, 11%). Mass (m/z): 750.0 [M+H]⁺. ¹H NMR (400 MHZ, DMSO-d₆) δ 11.72 (d, J=2.4 Hz, 1H), 11.08 (s, 1H), 8.08 (s, 1H), 7.79 (d, J=8.2 Hz, 1H), 7.49-7.44 (m, 2H), 7.40-7.34 (m, 3H), 7.33-7.26 (m, 2H), 5.08 (dd, J=12.8, 5.2 Hz, 1H), 4.16 (t, J=6.2 Hz, 2H), 4.07-3.98 (m, 4H), 3.77-3.67 (m, 4H), 3.62-3.57 (m, 2H), 2.90-2.48 (m, 4H), 2.19-2.13 (m, 1H), 2.04-1.98 (m, 3H), 0.82-0.79 (m, 2H), 0.62-0.58 (m, 2H).

### Example 96: 1-(7-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl) imidazo[1,2-a]pyridin-3-yl)dihydro pyrimidine-2,4 (1H,3H)-dione

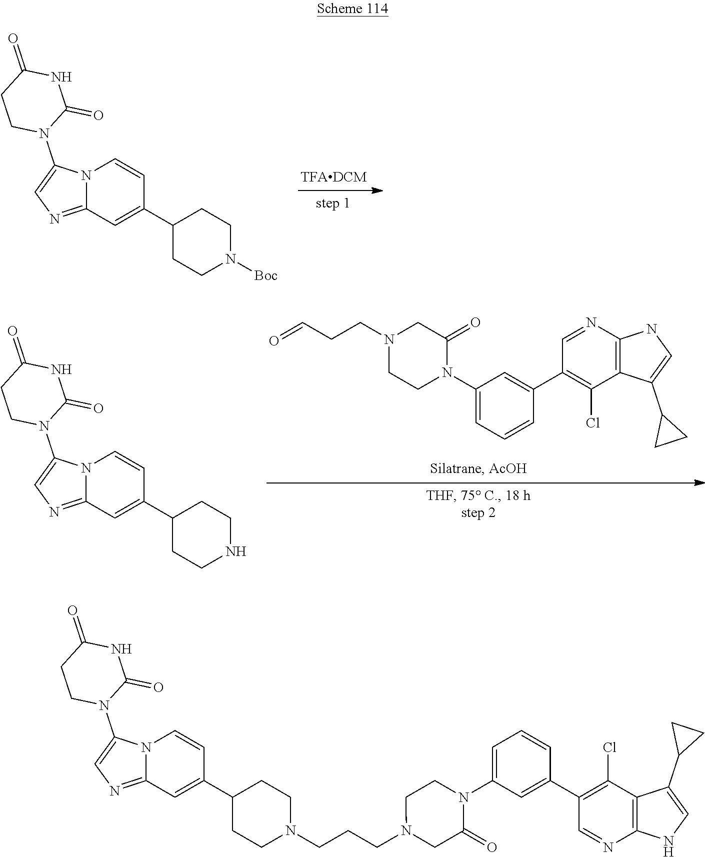 [SMILES: CC(C)(C)OC(=O)N1CCC(c2ccn3c(N4CCC(=O)NC4=O)cnc3c2)CC1 ; O=C1CCN(c2cnc3cc(C4CCNCC4)ccn23)C(=O)N1 ; O=CCCN1CCN(c2cccc(-c3cnc4[nH]cc(C5CC5)c4c3Cl)c2)C(=O)C1 ; O=C1CCN(c2cnc3cc(C4CCN(CCCN5CCN(c6cccc(-c7cnc8[nH]cc(C9CC9)c8c7Cl)c6)C(=O)C5)CC4)ccn23)C(=O)N1]

Step 1. 1-(7-(piperidin-4-yl) imidazo[1,2-a]pyridin-3-yl)dihydropyrimidine-2,4 (1H, 3H)-dione: Following General step B2, the desired product was obtained by suction filtration (120 mg, 97%) as brown oil. Mass (m/z): 314 [M+H]⁺.

Step 2. 1-(7-(1-(3-(4-(3-(4-chloro-3-cyclopropyl-1H-pyrrolo[2,3-b]pyridin-5-yl)phenyl)-3-oxopiperazin-1-yl) propyl)piperidin-4-yl) imidazo[1,2-a]pyridin-3-yl)dihydropyrimidine-2,4 (1H, 3H)-dione: Following General step M2, the desired product (22 mg, 4%) was obtained as a yellow solid. Mass (m/z): 720.1 [M+H]⁺. ¹H NMR (400 MHZ, DMSO-d₆) δ 11.78 (s, 1H), 10.85 (d, J=5.2 Hz, 1H), 8.72 (d, J=6.8 Hz, 1H), 8.13 (s, 1H), 7.74 (s, 1H), 7.66 (s, 1H), 7.54 (t, J=7.8 Hz, 1H), 7.49 (s, 1H), 7.46-7.38 (m, 2H), 7.32 (d, J=1.6 Hz, 1H), 7.27-6.98 (m, 1H), 3.85 (s, 4H), 3.69-3.67 (m, 2H), 3.23 (s, 3H), 3.13 (s, 5H), 2.80 (dd, J=19.2, 13.0 Hz, 4H), 2.26-1.93 (m, 6H), 1.57 (s, 2H), 0.89-0.81 (m, 2H), 0.65 (q, J=5.8 Hz, 2H).

### Example 97: 5-(2-(4-(3-(4-chloro-3-ethyl-1H-pyrrolo[2,3-b]pyridin-5-yl)phenyl)-3-oxo piperazin-1-yl)ethyl)-2-(2,6-dioxopiperidin-3-yl)isoindoline-1,3-dione

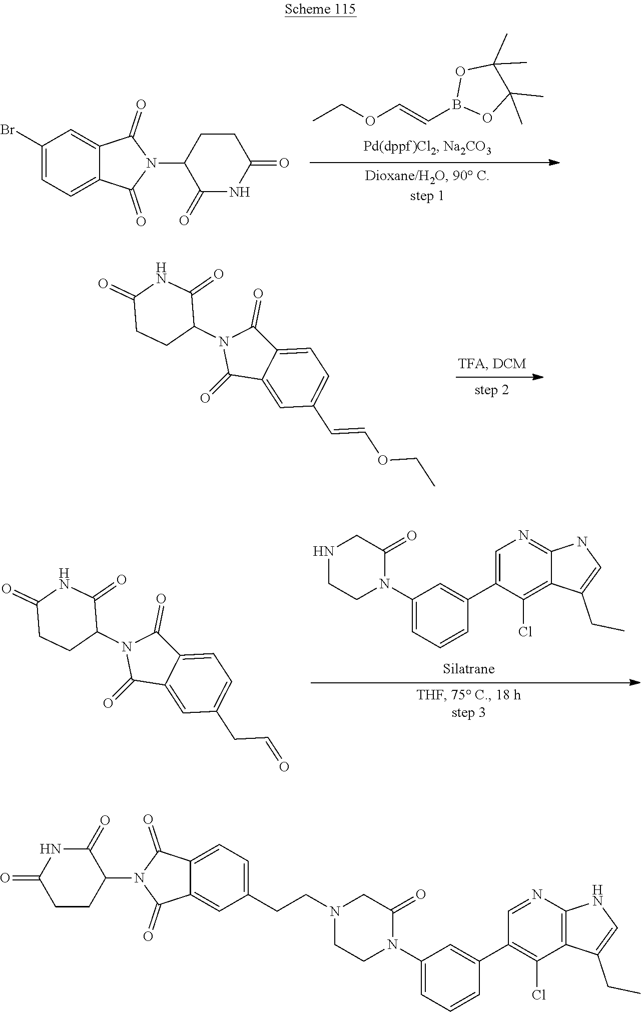 [SMILES: O=C1CCC(N2C(=O)c3ccc(Br)cc3C2=O)C(=O)N1 ; CCO/C=C/B1OC(C)(C)C(C)(C)O1 ; CCO/C=C/c1ccc2c(c1)C(=O)N(C1CCC(=O)NC1=O)C2=O ; O=CCc1ccc2c(c1)C(=O)N(C1CCC(=O)NC1=O)C2=O ; CCc1c[nH]c2ncc(-c3cccc(N4CCNCC4=O)c3)c(Cl)c12 ; CCc1c[nH]c2ncc(-c3cccc(N4CCN(CCc5ccc6c(c5)C(=O)N(C5CCC(=O)NC5=O)C6=O)CC4=O)c3)c(Cl)c12]

Step 1. Preparation of (E)-2-(2,6-dioxopiperidin-3-yl)-5-(2-ethoxyvinyl)isoindoline-1,3-dione: To a mixture of 5-bromo-2-(2,6-dioxopiperidin-3-yl) isoindole-1,3-dione (1 g, 2.96 mmol), 2-[(E)-2-ethoxyethenyl]-4,4,5,5-tetramethyl-1,3,2-dioxaborolane (881 mg, 4.44 mmol) and Na₂CO₃ (943 mg, 8.89 mmol) in 1.4-dioxan/H₂O (10:1, 20 mL) under N₂ was added Pd(dppf)Cl₂ (342 mg, 0.29 mmol). The reaction mixture was stirred at 90° C. for 18 hrs. The mixture was concentrated under reduced pressure, the residue was purified by column chromatography (DCM/MeOH=10:1) to give the desired product (800 mg, yield: 73%) as a brown solid. Mass (m/z): 329 [M+H]⁺.

Step 2. Preparation of 2-(2-(2,6-dioxopiperidin-3-yl)-1,3-dioxoisoindolin-5-yl) acetaldehyde: The solution was added to a solution of 2-(2,6-dioxopiperidin-3-yl)-5-[(E)-2-ethoxyethenyl]isoindole-1,3-dione (100 mg, 0.30 mmol) in DCM (1 mL) and TFA (0.1 mL). The reaction mixture was stirred at RT for 18 hrs. The solution was concentrated under reduced pressure to give the desired product (100 mg, yield: 98%) as a brown solid. Mass (m/z): 301.1 [M+H]⁺.

Step 3. Preparation of 5-(2-(4-(3-(4-chloro-3-ethyl-1H-pyrrolo[2,3-b]pyridin-5-yl)phenyl)-3-oxo piperazin-1-yl)ethyl)-2-(2,6-dioxopiperidin-3-yl)isoindoline-1,3-dione: Following General step M2, the desired product (35 mg, yield: 12%) was obtained as a white solid. Mass (m/z): 639 [M+H]⁺. ¹H NMR (400 MHZ, DMSO-d₆) δ 11.75 (d, J=2.2 Hz, 1H), 11.09 (s, 1H), 8.09 (d, J=6.4 Hz, 1H), 7.89-7.74 (m, 3H), 7.45 (dd, J=15.8, 8.0 Hz, 2H), 7.39-7.31 (m, 3H), 5.10 (dd, J=13.0, 5.4 Hz, 1H), 3.69 (s, 1H), 3.25 (d, J=4.8 Hz, 2H), 3.00 (s, 1H), 2.93-2.77 (m, 5H), 2.64-2.50 (m, 2H), 2.50-2.47 (m, 3H), 2.30-2.27 (m, 1H), 2.02 (dd, J=9.2, 3.8 Hz, 1H), 1.23 (t, J=7.4 Hz, 3H).

### Example 98: 1-(2-(2-(2,6-dioxopiperidin-3-yl)-1,3-dioxoisoindolin-5-yl)ethyl)piperidin-4-yl 4-(3-(4-chloro-3-cyclopropyl-1H-pyrrolo[2,3-b]pyridin-5-yl)phenyl)-3-oxopiperazine-1-carboxylate

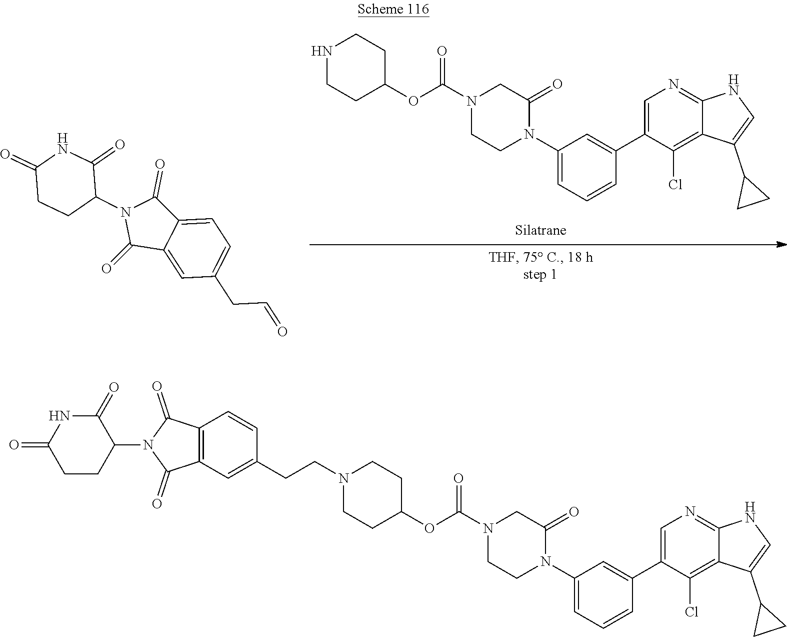 [SMILES: O=CCc1ccc2c(c1)C(=O)N(C1CCC(=O)NC1=O)C2=O ; O=C(OC1CCNCC1)N1CCN(c2cccc(-c3cnc4[nH]cc(C5CC5)c4c3Cl)c2)C(=O)C1 ; O=C1CCC(N2C(=O)c3ccc(CCN4CCC(OC(=O)N5CCN(c6cccc(-c7cnc8[nH]cc(C9CC9)c8c7Cl)c6)C(=O)C5)CC4)cc3C2=O)C(=O)N1]

Step 1. Preparation of 1-(2-(2-(2,6-dioxopiperidin-3-yl)-1,3-dioxoisoindolin-5-yl)ethyl)piperidin-4-yl 4-(3-(4-chloro-3-cyclopropyl-1H-pyrrolo[2,3-b]pyridin-5-yl)phenyl)-3-oxopiperazine-1-carboxylate: Following General step M2, the desired product (23 mg, yield: 8%) was obtained as a white solid. Mass (m/z): 778.1 [M+H]⁺. ¹H NMR (400 MHZ, DMSO-d₆) δ 11.73 (d, J=2.4 Hz, 1H), 11.09 (s, 1H), 8.09 (d, J=5.6 Hz, 1H), 7.82 (s, 2H), 7.73 (d, J=7.6 Hz, 1H), 7.50-7.44 (m, 2H), 7.40-7.34 (m, 2H), 7.27 (d, J=1.6 Hz, 1H), 5.10 (dd, J=12.8, 5.2 Hz, 1H), 4.20-4.06 (m, 2H), 3.83-3.67 (m, 4H), 3.00-2.82 (m, 4H), 2.64-2.53 (m, 3H), 2.48 (s, 5H), 2.43-2.36 (m, 2H), 2.29 (dt, J=3.6, 1.8 Hz, 1H), 2.23-2.11 (m, 1H), 2.07-1.98 (m, 1H), 1.88 (s, 1H), 0.83-0.79 (m, 2H), 0.63-0.58 (m, 2H).

### Example 99: 5-(4-(3-((4-(3-(4-chloro-3-ethyl-1H-pyrrolo[2,3-b]pyridin-5-yl)phenyl)-3-oxopiperazin-1-yl) sulfonyl) propyl)piperazin-1-yl)-2-(2,6-dioxopiperidin-3-yl)isoindoline-1,3-dione

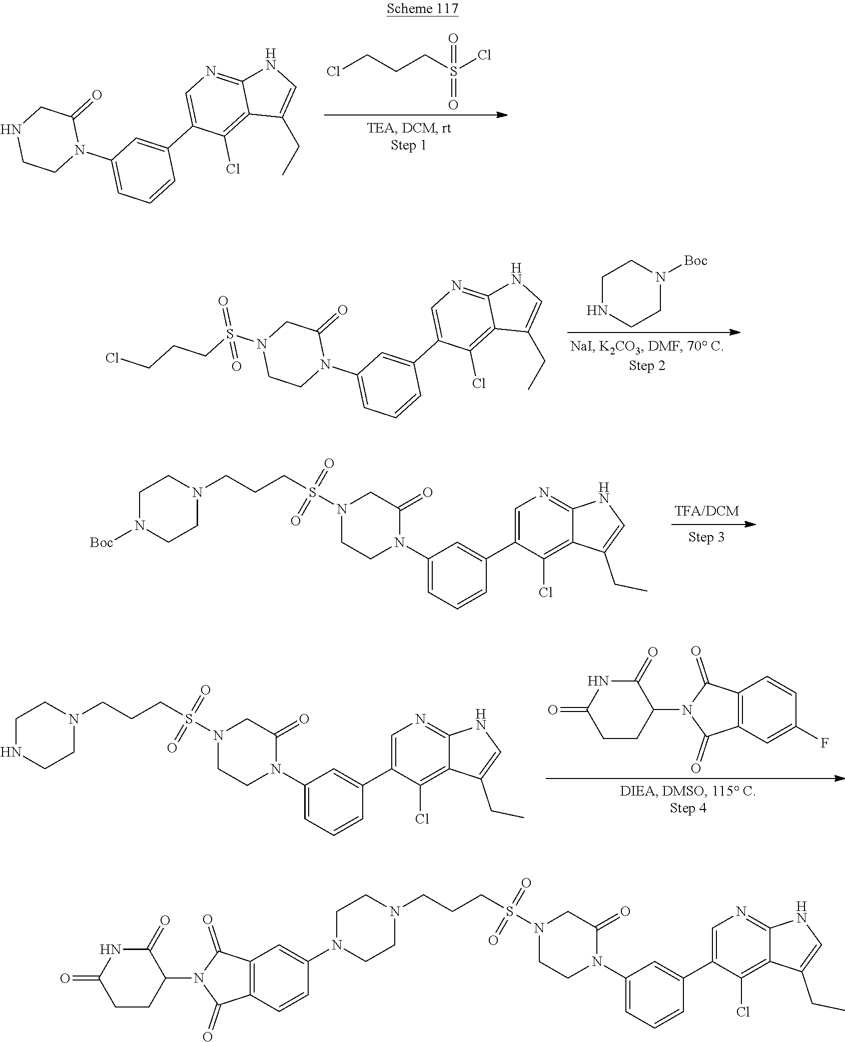

Step 1. Preparation of 1-(3-{4-chloro-3-ethyl-1H-pyrrolo[2,3-b]pyridin-3-yl}phenyl)-4-[(3-chloropropane) sulfonyl]piperazin-2-one: To a solution of 1-(3-{4-chloro-3-ethyl-1H-pyrrolo[2,3-b]pyridin-3-yl}phenyl)piperazin-2-one (100 mg, 0.28 mmol) and TEA (56 mg, 0.563 mmol) in DCM (5 mL) was added 3-chloropropane-1-sulfonyl chloride (54 mg, 0.309 mmol) dropwise. The reaction mixture was stirred at rt under N₂ for 3 hrs. Water (10 mL) was added and extracted with DCM (10 mL×2). The organic layer was washed with brine (10 mL×2) dried over Na₂SO₄. Then by filtration, the filtrate was concentrated to give the product 1-(3-{4-chloro-3-ethyl-1H-pyrrolo[2,3-b]pyridin-3-yl}phenyl)-4-[(3-chloropropane) sulfonyl]piperazin-2-one as a yellow solid (160 mg, 91%). Mass (m/z): 495.0 [M+H]⁺.

Step 2. (General Step N) Preparation of tert-butyl 4-(3-((4-(3-(4-chloro-3-ethyl-1H-pyrrolo[2,3-b]pyridin-5-yl)phenyl)-3-oxopiperazin-1-yl) sulfonyl) propyl)piperazine-1-carboxylate: To a solution of 1-(3-{4-chloro-3-ethyl-1H-pyrrolo[2,3-b]pyridin-3-yl}phenyl)-4-[(3-chloropropane) sulfonyl]piperazin-2-one (100 mg, 0.201 mmol), K₂CO₃ (40 mg, 0.403 mmol) and NaI (60 mg, 0.403 mmol) in DMF (5 mL) was added tert-butyl piperazin-1-yl formate (56 mg, 0.302 mmol). The reaction mixture was stirred at 70° C. under N₂ for 3 hrs. Water (15 mL) was added and extracted with EA (15 mL×2). The organic layer was washed with brine (15 mL×3), dried over Na₂SO₄ and concentrated. The residue was purified by prep-TLC (DCM/MeOH=10:1) to give the product tert-butyl 4-(3-((4-(3-(4-chloro-3-ethyl-1H-pyrrolo[2,3-b]pyridin-5-yl)phenyl)-3-oxopiperazin-1-yl) sulfonyl) propyl)piperazine-1-carboxylate as a brown solid (65 mg, 50%). Mass (m/z): 645.0 [M+H]⁺.

Step 3. Preparation of 1-(3-(4-chloro-3-ethyl-1H-pyrrolo[2,3-b]pyridin-5-yl)phenyl)-4-((3-(piperazin-1-yl) propyl) sulfonyl)piperazin-2-one: Following General step B2, the product was obtained as a brown solid (80 mg, purity: 60%). Mass (m/z): 545.0 [M+H]⁺.

Step 4. 5-(4-(3-((4-(3-(4-chloro-3-ethyl-1H-pyrrolo[2,3-b]pyridin-5-yl)phenyl)-3-oxopiperazin-1-yl) sulfonyl) propyl)piperazin-1-yl)-2-(2,6-dioxopiperidin-3-yl)isoindoline-1,3-dione: Following General step L, the product was obtained as a yellow solid (24 mg, 11%). Mass (m/z): 800.9 [M+H]⁺. ¹H NMR (400 MHZ, DMSO-d₆) δ 11.82 (s, 1H), 11.10 (s, 1H), 8.13 (s, 1H), 7.67 (d, J=8.4 Hz, 1H), 7.57-7.50 (m, 1H), 7.49 (s, 1H), 7.46-7.38 (m, 3H), 7.33 (s, 1H), 7.22 (d, J=8.8 Hz, 1H), 5.08 (dd, J=12.8, 5.4 Hz, 1H), 4.04 (s, 2H), 3.93-3.82 (m, 2H), 3.72-3.61 (m, 2H), 3.47 (s, 9H), 3.33-3.21 (m, 2H), 2.91 (dd, J=14.8, 7.3 Hz, 2H), 2.67-2.54 (m, 2H), 2.45 (d, J=7.0 Hz, 2H), 2.07-1.96 (m, 1H), 1.96-1.83 (m, 2H), 1.27 (t, J=7.4 Hz, 3H).

### Example 100: 3-ethyl-1H-pyrrolo 5-(4-(2-((4-(3-(4-chloro-3-ethyl-1H-pyrrolo[2,3-b]pyridin-5-yl)phenyl)-3-oxopiperazin-1-yl) sulfonyl)ethyl)piperazin-1-yl)-2-(2,6-dioxopiperidin-3-yl)isoindoline-1,3-dione

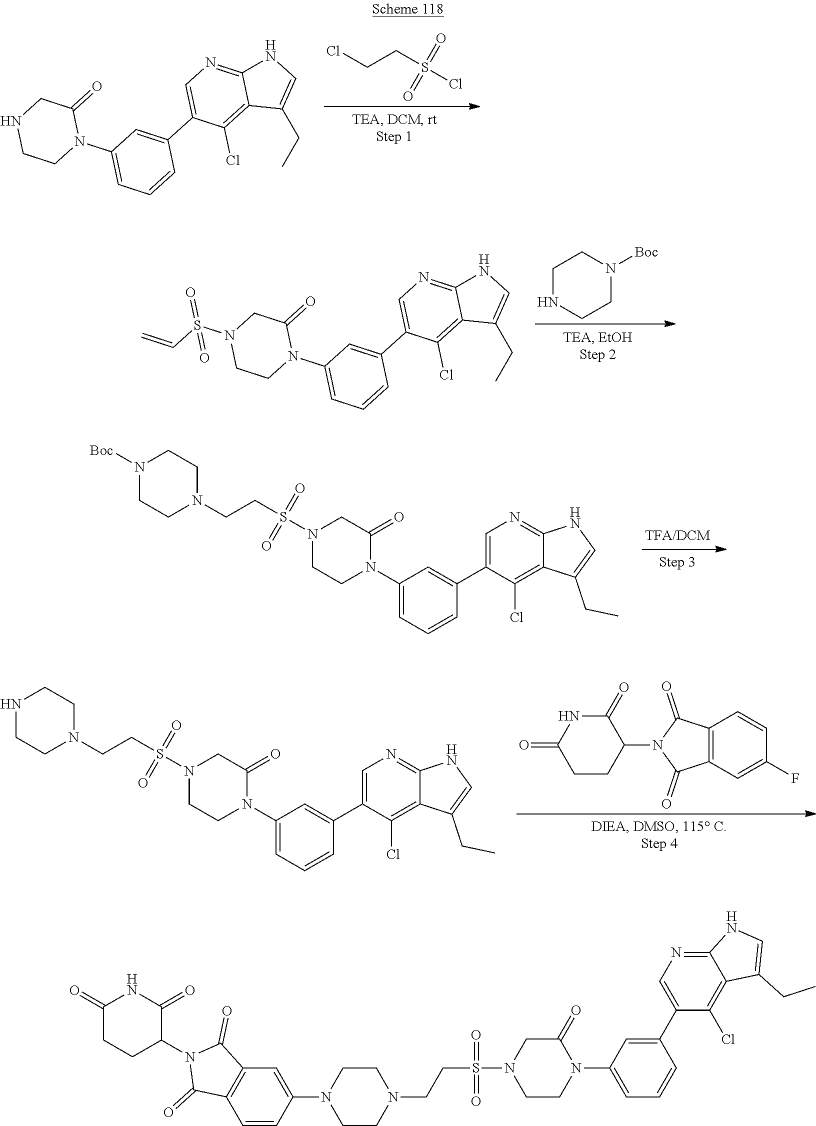

Step 1. Preparation of 1-(3-{4-chloro-3-ethyl-1H-pyrrolo[2,3-b]pyridin-3-yl}phenyl)-4-(ethenesulfonyl)piperazin-2-one: To a mixture of 1-(3-{4-chloro-3-ethyl-1H-pyrrolo[2,3-b]pyridin-3-yl}phenyl)piperazin-2-one (200 mg, 0.564 mmol) and TEA (200 mg, 1.97 mmol) in DCM (10 mL) was added 2-chloroethanesulfonyl chloride (91 mg, 0.564 mmol). The reaction mixture was stirred at 0° C. under N₂ for 2 hrs. The reaction mixture was concentrated under reduced pressure. The residue was purified by Flash Chromatography (PE/EA=0˜100%) to give the product 1-(3-{4-chloro-3-ethyl-1H-pyrrolo[2,3-b]pyridin-3-yl}phenyl)-4-(ethenesulfonyl)piperazin-2-one (170 mg, 61%) as a yellow solid. Mass (m/z): 444.8 [M+H]⁺.

Step 2. Preparation of tert-butyl (4-{2-[4-(3-{4-chloro-3-ethyl-1H-pyrrolo[2,3-b]pyridin-3-yl}phenyl)-3-oxopiperazine-1-sulfonyl]ethyl}piperazin-1-yl) formate: To a mixture of 1-(3-{4-chloro-3-ethyl-1H-pyrrolo[2,3-b]pyridin-3-yl}phenyl)-4-(ethenesulfonyl)piperazin-2-one (170 mg, 0.382 mmol) in EtOH (5 mL) was added TEA (116 mg, 1.15 mmol) and tert-butyl piperazin-1-yl formate (143 mg, 0.764 mmol), The reaction mixture was stirred at 80° C. under N₂ for 2 hrs. The reaction mixture was concentrated under reduced pressure. The residue was purified by Prep-TLC (DCM/MeOH=10:1) to give the product tert-butyl (4-{2-[4-(3-{4-chloro-3-ethyl-1H-pyrrolo[2,3-b]pyridin-3-yl}phenyl)-3-oxopiperazine-1-sulfonyl]ethyl}piperazin-1-yl) formate (170 mg, 63%) as a yellow solid. Mass (m/z): 630.8 [M+H]⁺.

Step 3. Preparation of 1-(3-{4-chloro-3-ethyl-1H-pyrrolo[2,3-b]pyridin-3-yl}phenyl)-4-{[2-(piperazin-1-yl) ethane]sulfonyl}piperazin-2-one: Following General step B2, the product (150 mg, 94%) was obtained as a brown solid. Mass (m/z): 531.0 [M+H]⁺.

Step 4. Preparation of 5-(4-{2-[4-(3-{4-chloro-3-ethyl-1H-pyrrolo[2,3-b]pyridin-3-yl}phenyl)-3-oxopiperazine-1-sulfonyl]ethyl}piperazin-1-yl)-2-(2,6-dioxopiperidin-3-yl) isoindole-1,3-dione: Following General step L, product 5-(4-{2-[4-(3-{4-chloro-3-ethyl-1H-pyrrolo[2,3-b]pyridin-3-yl}phenyl)-3-oxopiperazine-1-sulfonyl]ethyl}piperazin-1-yl)-2-(2,6-dioxopiperidin-3-yl) isoindole-1,3-dione (55 mg, 24%) was obtained as a yellow solid.

Mass (m/z): 786.7 [M+H]⁺. ¹H NMR (400 MHZ, DMSO-d₆) δ 11.81 (d, J=2.2 Hz, 1H), 11.11 (s, 1H), 8.13 (s, 1H), 7.76 (d, J=8.4 Hz, 1H), 7.56-7.48 (m, 3H), 7.45-7.35 (m, 4H), 5.10 (dd, J=13.0, 5.2 Hz, 1H), 4.10 (s, 3H), 3.93-3.88 (m, 3H), 3.81 (s, 3H), 3.73-3.68 (m, 3H), 3.55 (s, 3H), 2.92 (d, J=7.0 Hz, 3H), 2.68-2.53 (m, 3H), 2.06-1.99 (m, 1H), 1.27 (t, J=7.4 Hz, 3H).

### Example 101: 5-(4-(3-((4-(3-(4-chloro-3-cyclopropyl-1H-pyrrolo[2,3-b]pyridin-5-yl)phenyl)-3-oxopiperazin-1-yl) sulfonyl) propyl)piperazin-1-yl)-2-(2,6-dioxopiperidin-3-yl)-6-fluoroisoindoline-1,3-dione

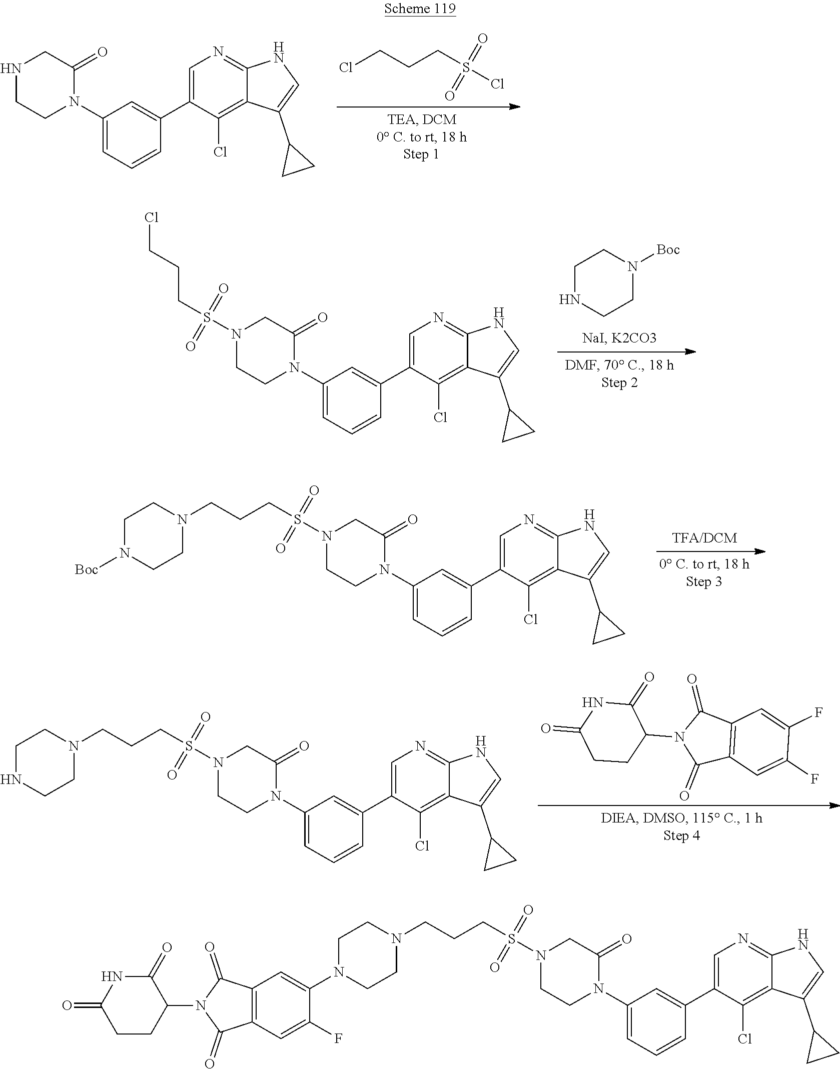

Step 1. Preparation of 1-(3-(4-chloro-3-cyclopropyl-1H-pyrrolo[2,3-b]pyridin-5-yl)phenyl)-4-((3-chloropropyl) sulfonyl)piperazin-2-one: To a solution of 1-(3-{4-chloro-3-cyclopropyl-1H-pyrrolo[2,3-b]pyridin-3-yl}phenyl)piperazin-2-one (600 mg, 1.64 mmol) and TEA (330 mg, 3.27 mmol) in DCM (30 mL) was added 3-chloropropane-1-sulfonyl chloride (347.5 mg, 1.96 mmol) dropwise. The reaction mixture was stirred at rt under N₂ for 3 h. Water (20 mL) was added and extracted with DCM (20 mL×2). The DCM layer was washed with brine (20 mL×2) dried over Na₂SO₄ and concentrated to give the crude product (780 mg, yield: 89.3%) as a white solid. Mass (m/z): 507.0 [M+H]⁺.

Step 2. Preparation of tert-butyl 4-(3-((4-(3-(4-chloro-3-cyclopropyl-1H-pyrrolo[2,3-b]pyridin-5-yl)phenyl)-3-oxopiperazin-1-yl) sulfonyl) propyl)piperazine-1-carboxylate: Following General step N, the desired product (850 mg, yield: 79.81%) was obtained as a brown solid. Mass (m/z): 657.3 [M+H]⁺.

Step 3. Preparation of 1-(3-(4-chloro-3-cyclopropyl-1H-pyrrolo[2,3-b]pyridin-5-yl)phenyl)-4-((3-(piperazin-1-yl) propyl) sulfonyl)piperazin-2-one: Following General step B2, the desired product (850 mg, yield: 84.5%) was obtained as a brown solid. Mass (m/z): 557.0 [M+H]⁺.

Step 4. Preparation of 5-(4-(3-((4-(3-(4-chloro-3-cyclopropyl-1H-pyrrolo[2,3-b] pyridin-5-yl)phenyl)-3-oxopiperazin-1-yl) sulfonyl) propyl)piperazin-1-yl)-2-(2,6-dioxopiperidin-3-yl)-6-fluoroisoindoline-1,3-dione: Following General step L, the desired product (400 mg, yield: 29.96%) was obtained as a yellow solid. Mass (m/z): 830.8 [M+18]⁺. ¹H NMR (400 MHZ, DMSO-d₆) δ 11.77 (d, J=2.3 Hz, 1H), 11.13 (s, 1H), 8.13 (d, J=4.5 Hz, 1H), 7.73 (d, J=11.3 Hz, 1H), 7.58-7.49 (m, 2H), 7.48-7.35 (m, 3H), 7.31 (d, J=1.7 Hz, 1H), 5.11 (dd, J=12.8, 5.4 Hz, 1H), 4.05 (s, 2H), 3.91-3.81 (m, 2H), 3.73-3.59 (m, 2H), 3.27 (d, J=29.4 Hz, 8H), 2.87 (dd, J=16.8, 5.1 Hz, 1H), 2.69-2.51 (m, 6H), 2.24-2.15 (m, 1H), 2.08-1.99 (m, 1H), 1.91 (s, 2H), 0.88-0.80 (m, 2H), 0.67-0.58 (m, 2H).

### Example 102: 3-(4-(1-(3-((4-(3-(4-chloro-3-cyclopropyl-1H-pyrrolo[2,3-b]pyridin-5-yl)phenyl)-3-oxopiperazin-1-yl) sulfonyl) propyl)piperidin-4-yl)phenyl)piperidine-2,6-dione

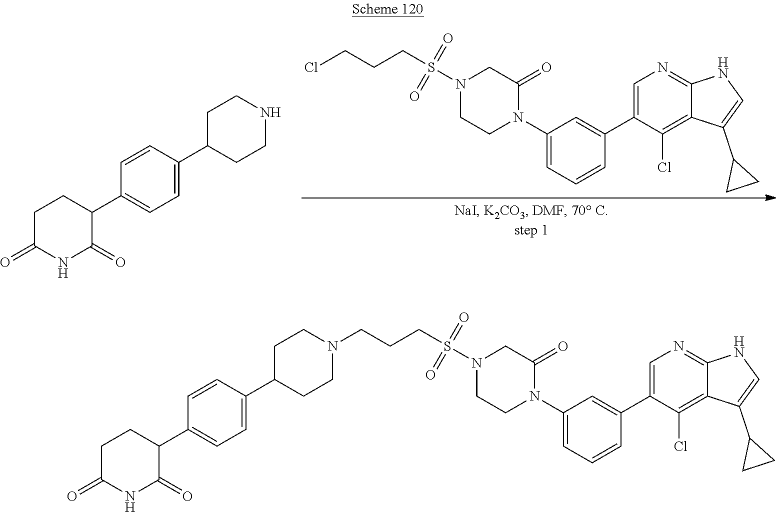 [SMILES: O=C1CCC(c2ccc(C3CCNCC3)cc2)C(=O)N1 ; O=C1CN(S(=O)(=O)CCCCl)CCN1c1cccc(-c2cnc3[nH]cc(C4CC4)c3c2Cl)c1 ; O=C1CCC(c2ccc(C3CCN(CCCS(=O)(=O)N4CCN(c5cccc(-c6cnc7[nH]cc(C8CC8)c7c6Cl)c5)C(=O)C4)CC3)cc2)C(=O)N1]

Step 1. Preparation of 3-[4-(1-{3-[4-(3-{4-chloro-3-cyclopropyl-1H-pyrrolo[2,3-b]pyridin-3-yl}phenyl)-3-oxopiperazine-1-sulfonyl]propyl}piperidin-4-yl)phenyl]piperidine-2,6-dione: Following General step N, the desired product (1.83 g, 14%) was obtained as a light yellow solid. Mass (m/z): 742.7 [M+H]⁺. ¹H NMR (400 MHZ, DMSO-d₆) δ 11.73 (s, 1H), 10.78 (s, 1H), 8.11 (s, 3H), 8.09 (s, 1H), 7.49 (dd, J=15.2, 7.4 Hz, 2H), 7.38 (d, J=7.2 Hz, 2H), 7.27 (d, J=1.8 Hz, 1H), 7.14 (d, J=8.2 Hz, 2H), 7.08 (d, J=8.2 Hz, 2H), 4.00 (s, 2H), 3.83 (m, 2H), 3.77 (m, 1H), 3.63 (m, 2H), 3.22 (s, 2H), 2.93 (d, J=8.6 Hz, 2H), 2.62 (d, J=7.0 Hz, 1H), 2.42 (s, 4H), 2.15 (d, J=8.2 Hz, 2H), 2.00 (d, J=9.2 Hz, 3H), 1.86 (s, 2H), 1.62 (m, 4H), 0.80 (dt, J=8.2, 2.8 Hz, 2H), 0.60 (d, J=3.6 Hz, 2H).

### Example 103: 3-(5-(4-(3-((4-(3-(4-chloro-3-cyclopropyl-1H-pyrrolo[2,3-b]pyridin-5-yl)phenyl)-3-oxopiperazin-1-yl) sulfonyl) propyl)piperazin-1-yl)-1-oxoisoindolin-2-yl)piperidine-2,6-dione

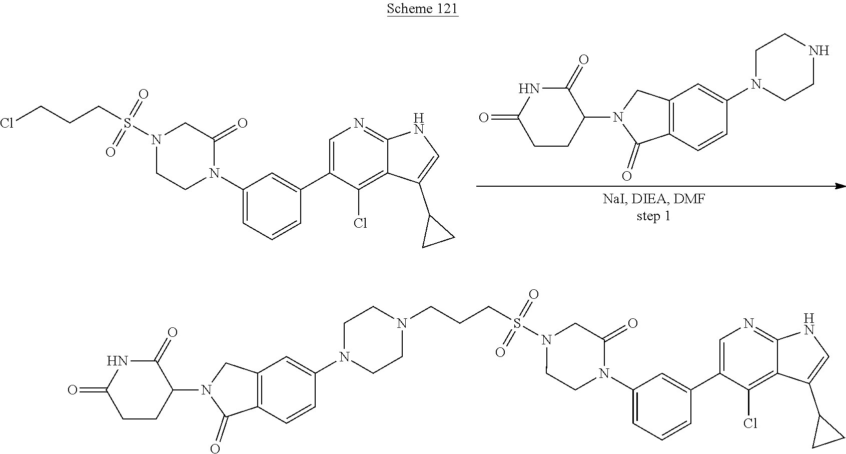 [SMILES: O=C1CN(S(=O)(=O)CCCCl)CCN1c1cccc(-c2cnc3[nH]cc(C4CC4)c3c2Cl)c1 ; O=C1CCC(N2Cc3cc(N4CCNCC4)ccc3C2=O)C(=O)N1 ; O=C1CCC(N2Cc3cc(N4CCN(CCCS(=O)(=O)N5CCN(c6cccc(-c7cnc8[nH]cc(C9CC9)c8c7Cl)c6)C(=O)C5)CC4)ccc3C2=O)C(=O)N1]

Step 1. Preparation of 3-(5-(4-(3-((4-(3-(4-chloro-3-cyclopropyl-1H-pyrrolo[2,3-b]pyridin-5-yl)phenyl)-3-oxopiperazin-1-yl) sulfonyl) propyl)piperazin-1-yl)-1-oxoisoindolin-2-yl)piperidine-2,6-dione: To a solution of 3-[1-oxo-5-(piperazin-1-yl)-3H-isoindol-2-yl]piperidine-2,6-dione (65 mg, 0.20 mmol), NaI (29 mg, 0.20 mmol) and DIEA (76 g, 0.60 mmol) in DMF (5 mL) was added 1-(3-{4-chloro-3-cyclopropyl-1H-pyrrolo[2,3-b]pyridin-3-yl}phenyl)-4-[(3-chloropropane) sulfonyl]piperazin-2-one (100 mg, 0.20 mmol). The reaction mixture was stirred at 120° C. under N₂ for 5 hrs. The mixture was concentrated and the residue was purified by Prep-HPLC [Gemini-C₁₈, 150×21.2 mm, 5 um; ACN-H₂O (0.1% FA), 20-40] to give the product as a white solid (17.5 mg, 11%). Mass (m/z): 798.7 [M+H]⁺. ¹H NMR (400 MHz, DMSO-d₆) δ 11.78 (s, 1H), 10.96 (s, 1H), 8.13 (d, J=2.2 Hz, 1H), 7.53 (dd, J=16.2, 8.2 Hz, 3H), 7.43 (d, J=6.4 Hz, 2H), 7.32 (s, 1H), 7.07 (s, 2H), 5.05 (dd, J=13.2, 4.8 Hz, 1H), 4.27 (dd, J=49.8, 16.9 Hz, 3H), 4.05 (s, 2H), 3.87 (s, 2H), 3.68 (s, 2H), 2.98-2.86 (m, 2H), 2.67-2.61 (m, 4H), 2.39-2.33 (m, 6H), 2.20 (s, 2H), 1.97 (s, 4H), 0.84 (d, J=7.8 Hz, 2H), 0.64 (d, J=3.8 Hz, 2H).

### Example 104: 3-cyclopropyl-1H-pyrrolo3-((4-(1-(3-((4-(3-(4-chloro-3-cyclopropyl-1H-pyrrolo[2,3-b]pyridin-5-yl)phenyl)-3-oxopiperazin-1-yl) sulfonyl) propyl)piperidin-4-yl)phenyl)amino)piperidine-2,6-dione

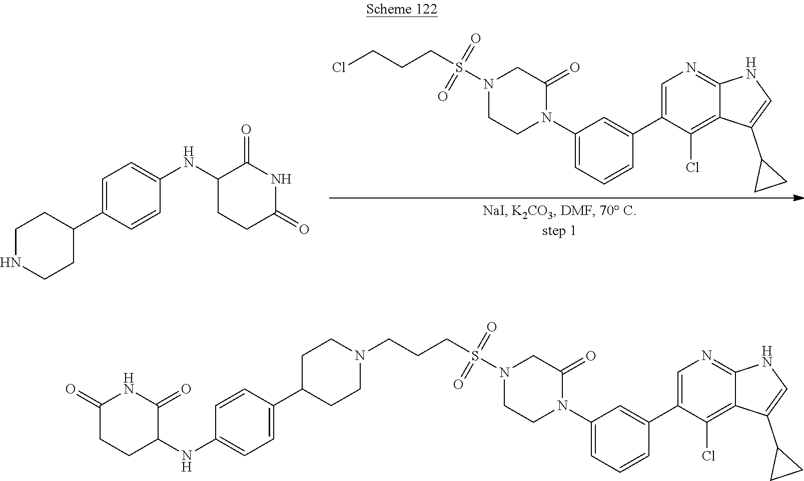 [SMILES: O=C1CCC(Nc2ccc(C3CCNCC3)cc2)C(=O)N1 ; O=C1CN(S(=O)(=O)CCCCl)CCN1c1cccc(-c2cnc3[nH]cc(C4CC4)c3c2Cl)c1 ; O=C1CCC(Nc2ccc(C3CCN(CCCS(=O)(=O)N4CCN(c5cccc(-c6cnc7[nH]cc(C8CC8)c7c6Cl)c5)C(=O)C4)CC3)cc2)C(=O)N1]

Step 1. Preparation of 3-{[4-(1-{3-[4-(3-{4-chloro-3-cyclopropyl-1H-pyrrolo[2,3-b] pyridin-3-yl}phenyl)-3-oxopiperazine-1-sulfonyl]propyl}piperidin-4-yl)phenyl]amino}piperidine-2,6-dione: Following General step N, the desired product (5 mg, 1.75%) was obtained as a green solid. Mass (m/z): 758.3 [M+H]⁺. ¹H NMR (400 MHZ, DMSO-d₆) δ 11.76 (s, 1H), 10.79 (s, 1H), 9.14 (s, 1H), 8.12 (s, 1H), 7.50 (dt, J=31.0, 7.0 Hz, 4H), 7.31 (s, 1H), 6.97 (m, 2H), 6.64 (d, J=8.4 Hz, 2H), 4.28 (dd, J=11.4, 4.6 Hz, 1H), 4.04 (m, 2H), 3.88 (d, J=5.6 Hz, 2H), 3.69 (d, J=4.9 Hz, 2H), 3.22 (s, 2H), 2.68 (d, J=9.2 Hz, 2H), 2.33 (s, 1H), 2.15 (m, 4H), 1.97 (m, 4H), 1.80 (dd, J=26.8, 11.6 Hz, 2H), 1.45 (s, 1H), 1.23 (s, 4H), 0.85 (m, 2H), 0.65 (t, J=4.6 Hz, 2H).

### Example 105: 3-((4-(4-(3-((4-(3-(4-chloro-3-cyclopropyl-1H-pyrrolo[2,3-b]pyridin-5-yl)phenyl)-3-oxopiperazin-1-yl) sulfonyl) propyl)piperazin-1-yl)-3-fluorophenyl)amino)piperidine-2,6-dione

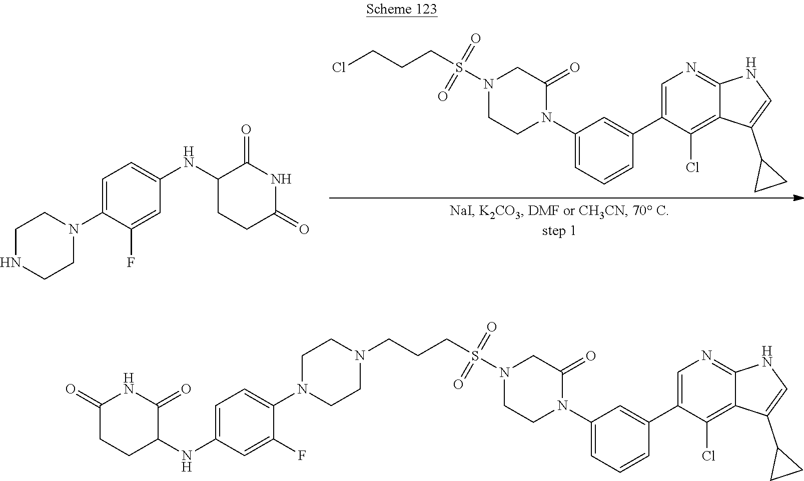 [SMILES: O=C1CCC(Nc2ccc(N3CCNCC3)c(F)c2)C(=O)N1 ; O=C1CN(S(=O)(=O)CCCCl)CCN1c1cccc(-c2cnc3[nH]cc(C4CC4)c3c2Cl)c1 ; O=C1CCC(Nc2ccc(N3CCN(CCCS(=O)(=O)N4CCN(c5cccc(-c6cnc7[nH]cc(C8CC8)c7c6Cl)c5)C(=O)C4)CC3)c(F)c2)C(=O)N1]

Step 1. Preparation of 3-{[4-(4-{3-[4-(3-{4-chloro-3-cyclopropyl-1H-pyrrolo[2,3-b]pyridin-3-yl}phenyl)-3-oxopiperazine-1-sulfonyl]propyl}piperazin-1-yl)-3-fluoro phenyl]amino}piperidine-2,6-dione: Following General step N, the desired product (12 mg, 1.55%) was obtained as a green solid. Mass (m/z): 777.3 [M+H]⁺. ¹H NMR (400 MHZ, DMSO-d6) δ 11.77 (s, 1H), 10.80 (s, 1H), 9.41 (s, 1H), 8.13 (s, 1H), 7.52 (m, 2H), 7.43 (d, J=7.6 Hz, 2H), 7.32 (s, 1H), 6.89 (m, 1H), 6.54 (d, J=14.8 Hz, 1H), 6.45 (d, J=8.6 Hz, 1H), 4.28 (d, J=7.6 Hz, 1H), 4.07 (s, 2H), 3.88 (d, J=5.4 Hz, 2H), 3.69 (d, J=5.0 Hz, 2H), 3.59 (d, J=11.2 Hz, 2H), 2.95 (m, 4H), 2.18 (dd, J=14.6, 6.6 Hz, 4H), 2.02 (m, 4H), 1.23 (s, 5H), 0.84 (d, J=6.4 Hz, 2H), 0.64 (d, J=4.0 Hz, 2H).

### Example 106: 5-{4-[3-(4-{3-[4-chloro-3-(2,2-difluoroethyl)-1H-pyrrolo[2,3-b]pyridin-3-yl]phenyl}-3-oxopiperazine-1-sulfonyl) propyl]piperazin-1-yl}-2-(2,6-dioxopiperidin-3-yl) isoindole-1,3-dione

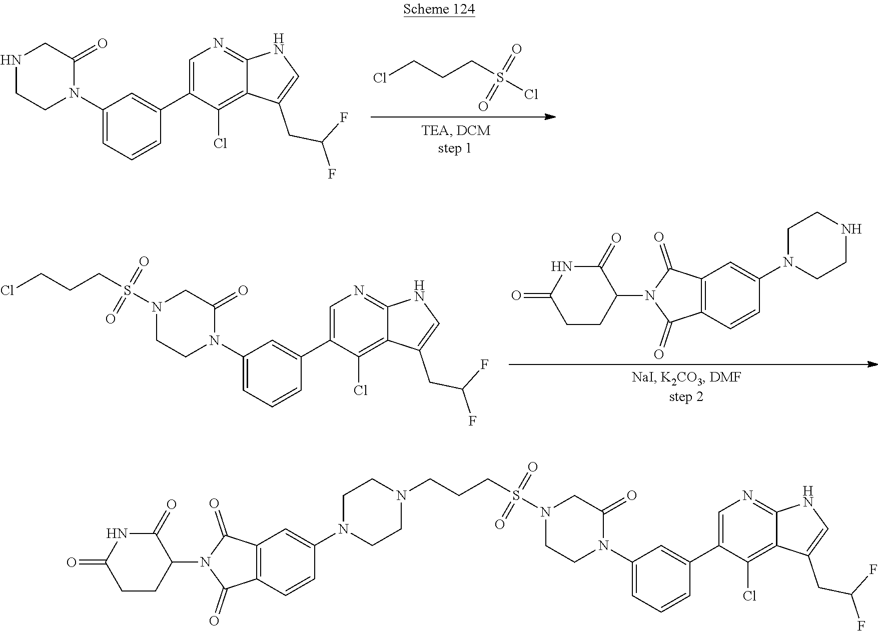 [SMILES: O=C1CNCCN1c1cccc(-c2cnc3[nH]cc(CC(F)F)c3c2Cl)c1 ; O=S(=O)(Cl)CCCCl ; O=C1CN(S(=O)(=O)CCCCl)CCN1c1cccc(-c2cnc3[nH]cc(CC(F)F)c3c2Cl)c1 ; O=C1CCC(N2C(=O)c3ccc(N4CCNCC4)cc3C2=O)C(=O)N1 ; O=C1CCC(N2C(=O)c3ccc(N4CCN(CCCS(=O)(=O)N5CCN(c6cccc(-c7cnc8[nH]cc(CC(F)F)c8c7Cl)c6)C(=O)C5)CC4)cc3C2=O)C(=O)N1]

Step 1. Preparation of 1-{3-[4-chloro-3-(2,2-difluoroethyl)-1H-pyrrolo[2,3-b]pyridin-3-yl]phenyl}-4-[(3-chloropropane) sulfonyl]piperazin-2-one: To a solution of 1-{3-[4-chloro-3-(2,2-difluoroethyl)-1H-pyrrolo[2,3-b]pyridin-3-yl]phenyl}piperazin-2-one (150 mg, 0.38 mmol) in DCM (10 mL) was added TEA (193 mg, 1.92 mmol). The reaction mixture was stirred under nitrogen at 25° C. for 30 min. Then 3-chloropropane-1-sulfonyl chloride (74 mg, 0.42 mmol) was slowly added at 0° C. The reaction mixture was stirred at 25° C. under nitrogen for 3 hrs. The solvent was removed under reduced pressure and the residue was purified by Combiflash (DCM/MeOH=95:5) to give the product as a yellow solid (130 mg, 55%). Mass (m/z): 531.1 [M+H]⁺.

Step 2. Preparation of 5-{4-[3-(4-{3-[4-chloro-3-(2,2-difluoroethyl)-1H-pyrrolo[2,3-b]pyridin-3-yl]phenyl}-3-oxopiperazine-1-sulfonyl) propyl]piperazin-1-yl}-2-(2,6-dioxopiperidin-3-yl) isoindole-1,3-dione: Following General step N, the desired product (15 mg, 7%) was obtained as a yellow solid. Mass (m/z): 837.3 [M+H]⁺. ¹H NMR (400 MHZ, DMSO-d₆) δ 12.11 (d, J=9.4 Hz, 1H), 8.79 (s, 1H), 8.17 (s, 1H), 7.73 (t, J=9.4 Hz, 1H), 7.59 (d, J=2.2 Hz, 1H), 7.50 (m, 2H), 7.42 (d, J=7.8 Hz, 3H), 7.29 (d, J=8.4 Hz, 1H), 6.42-6.15 (m, 1H), 5.20 (dd, J=12.8, 5.4 Hz, 1H), 4.03 (s, 2H), 3.83 (m, 4H), 3.64 (d, J=4.2 Hz, 8H), 3.51 (d, J=4.2 Hz, 2H), 3.23 (m, 6H), 2.66 (d, J=7.6 Hz, 2H), 1.99 (m, 2H).

### General Assay Procedures:

### 1. HPK1 Enzyme Inhibition Assay

The compound was dissolved in 100% DMSO at the concentration of 10 mM. The HPK1 protein was purchased from Signal Chem (M23-11G-10). 2.5 μL per well of 2×HPK1 protein was added to assay plate containing the test compound, centrifuged at 1500 rpm for 1 minute, and then incubated at 25° C. for 60 minutes. MBP protein was purchased from Signal Chem (M42-51N) and ATP was purchased from Promega (V9102). The two were added 2.5 μL per well mixture of 2×MBP (0.2 ug/ul) and ATP (20 μM), centrifuged at 1500 rpm for 1 minute, then incubated at 25° C. for 60 minutes. Then added 5 μL of ADP-Glo from Promega (V9102) to the assay plate and depleted the unconsumed ATP for 60 minutes. Then centrifuged at 1500 rpm for 1 minute and incubated at 25° C. for 60 minutes. Finally, 10 μL of the kinase assay reagent from Promega (V9102) was added to the assay plate to convert ADP to ATP, centrifuged at 1500 rpm for 1 minute, incubate at 25° C. for 40 minutes. After 40 minutes incubation, the fluorescence was determined. Based on the results, the IC₅₀ value of the compound was calculated. The results of IC₅₀ are shown in the following Table 2:

+++: IC₅₀<=10 nM; ++: 10 nM<IC₅₀<=100 nM; +: 100 nM<IC₅₀<=1 mM; NA: not active, IC₅₀>1 mM

TABLE 2
Results of compounds in HPK1 enzyme inhibition assays

| | HPK1 Enzyme |
|---|---|
| Example | inhibition IC₅₀ |
| 1 | ++ |
| 2 | + |
| 3 | +++ |
| 4 | ++ |
| 5 | ++ |
| 6 | ++ |
| 7 | + |
| 8 | + |
| 9 | ++ |
| 10 | + |
| 11 | + |
| 12 | ++ |
| 13 | ++ |
| 14 | ++ |
| 15 | ++ |
| 16 | +++ |
| 17 | + |
| 18 | ++ |
| 19 | ++ |
| 20 | ++ |
| 21 | ++ |
| 22 | ++ |
| 23 | ++ |
| 24 | ++ |
| 25 | ++ |
| 26 | ++ |
| 27 | ++ |
| 28 | ++ |
| 29 | ++ |
| 30 | ++ |
| 31 | ++ |
| 32 | ++ |
| 33 | ++ |
| 34 | ++ |
| 35 | ++ |
| 36 | ++ |
| 37 | NA |
| 38 | ++ |
| 39 | + |
| 40 | ++ |
| 41 | ++ |
| 42 | ++ |
| 43 | + |
| 44 | ++ |
| 45 | ++ |
| 46 | +++ |
| 47 | +++ |
| 48 | ++ |
| 49 | + |
| 50 | +++ |
| 51 | ++ |
| 52 | ++ |
| 53 | ++ |
| 54 | +++ |
| 55 | + |
| 56 | +++ |
| 57 | + |
| 58 | ++ |
| 59 | ++ |
| 60 | ++ |
| 61 | + |
| 62 | ++ |
| 63 | +++ |
| 64 | +++ |
| 65 | ++ |
| 66 | +++ |
| 67 | ++ |
| 68 | ++ |
| 69 | +++ |
| 70 | ++ |
| 71 | +++ |
| 72 | ++ |
| 73 | +++ |
| 74 | +++ |
| 75 | +++ |
| 76 | ++ |
| 77 | +++ |
| 78 | +++ |
| 79 | +++ |
| 80 | +++ |
| 81 | +++ |
| 82 | +++ |
| 83 | +++ |
| 84 | +++ |
| 85 | +++ |
| 86 | +++ |
| 87 | +++ |
| 88 | ++ |
| 89 | ++ |
| 90 | ++ |
| 91 | ++ |
| 92 | +++ |
| 93 | +++ |
| 94 | ++ |
| 95 | ++ |
| 96 | +++ |
| 97 | +++ |
| 98 | ++ |
| 99 | ++ |
| 100 | ++ |
| 101 | ++ |
| 102 | +++ |
| 103 | +++ |
| 104 | +++ |
| 105 | +++ |
| 106 | +++ |

2. HPK1 p-SLP-76 Inhibition Assay

Human PBMC were purchased from OriCells (FPB004F-C). Before compound treatment, PBMC were starved in PRMI-1640 medium supplemented with 0.5% FBS overnight. The cell density was adjusted to 1*10\hat{ }7 cells/mL in assay medium (RPMI-1640 no phenol red with 0.5% FBS) and 16 μL cell suspension was transferred per well to a 384-well plate (Corning #3765). 6× solution of test compound was prepared in assay medium and 4 μL was added per well to the 384-well plate. The plate was then incubated at 37° C., 5% CO₂ for 6 h. PBMC in 384-well plate were stimulated with anti-CD3 (BD Biosciences #555329, final conc. is 10 ug/mL) for 20 min at 37° C., 5% CO₂ by adding 4 μL 6× antibody solution per well. Phospho-SLP76 HTRF kit was purchased from Cisbio (63ADK076PEH). 8 μL 4× Lysis Buffer was added to 384-well plate after anti-CD3 stimulation. The plate was centrifuged at 1,000 rpm for 1 min, shaken at 250 rpm for 60 min and centrifuged again at 1,000 rpm for 5 min. Afterwards the cell lysates were transferred to the assay plate (Greiner #784075) with 16 μL per well. 4 μL detection antibody mix per well prepared in Detection Buffer was added to the assay plate, followed by centrifugation at 1,000 rpm for 1 min and incubation at 25° C. overnight. The fluorescence emission at two different wavelengths (665 nm and 620 nm) was read by an HTRF compatible microplate reader and the IC₅₀ value of the compound was calculated.

A: IC₅₀<=300 nM; B: 300 nM<IC₅₀<=3 mM; C: 3 mM<IC₅₀<=10 mM; NA: not active, IC₅₀>10 mM

TABLE 3
Results of HPK1 p-SLP-76 inhibition by
selected compounds in human PBMC

| Example | p-SLP-76 inhibition IC₅₀ |
|---|---|
| 1 | C |
| 12 | C |
| 16 | A |
| 25 | B |
| 28 | B |
| 35 | B |
| 36 | A |
| 43 | B |
| 50 | NA |
| 54 | A |
| 56 | B |
| 58 | B |
| 63 | A |
| 69 | B |
| 70 | B |
| 71 | A |
| 72 | B |
| 73 | A |
| 77 | A |
| 78 | B |
| 79 | A |
| 80 | A |
| 81 | A |
| 82 | A |
| 83 | A |
| 84 | A |
| 85 | B |
| 86 | B |
| 87 | A |
| 88 | NA |
| 91 | NA |
| 92 | B |
| 93 | B |
| 99 | A |
| 101 | A |
| 102 | B |
| 103 | A |
| 104 | A |
| 105 | A |
| 106 | B |

### 3. Western Blots

HPK1 protein degradation in mouse pan T cell: the CD3+ T cell population was isolated from spleenocytes of mice by using Pan T cell isolation kit from Miltenyi Biotec (130-095-130) following manufacture's instruction. The isolated T cells were then incubated with a variety of concentrations of compound. After incubation for 18 h, the cells were collected and lysed. The protein concentration was determined by BCA protein assay kit from Thermo (23227). The HPK1 protein level was determined by western blots, using anti-human HPK1 polyclonal antibody from CST (4472). Proteins were loaded into each well of the pre-casting gels and subjected to electrophoretic separation by SDS-PAGE. The protein resolved by SDS-PAGE were transferred to PVDF, blocked by 5% skim milk and probed with anti-human HPK1 antibody or β-actin antibody from CST (3700S), using following standard western blotting procedure. The degradation results are shown in the following Table 4

TABLE 4
Results of HPK1 protein degradation in mouse panT cell

| Example | DC₅₀ (nM) | Dmax (%) |
|---|---|---|
| 16 | 325 | 80 |
| 24 | 500 | 91 |
| 25 | 100 | 96 |
| 26 | 200 | 91 |
| 27 | 31 | 95 |
| 29 | 100 | 80 |
| 30 | 2000 | 80 |
| 31 | 109 | 95 |
| 32 | 848 | 90 |
| 33 | 8000 | 60 |
| 35 | 7 | 95 |
| 36 | 1 | 95 |
| 38 | 202 | 90 |
| 39 | 877 | 80 |
| 40 | 76 | 95 |
| 41 | 43 | 95 |
| 42 | 493 | 95 |
| 43 | 101 | 95 |
| 44 | 7000 | 50 |
| 45 | 100 | 90 |
| 46 | 1 | 90 |
| 47 | 62 | 95 |
| 48 | 9.5 | 95 |
| 50 | 1400 | 80 |
| 51 | 0.1 | 95 |
| 52 | 150 | 95 |
| 53 | 209 | 95 |
| 54 | 18 | 95 |
| 55 | 49 | 95 |
| 56 | 4.3 | 95 |
| 57 | >10000 | 0 |
| 58 | 120 | 90 |
| 59 | 1300 | 60 |
| 61 | 42000 | 50 |
| 62 | 119 | 95 |
| 73 | 450 | 95 |
| 74 | 800 | 91 |
| 75 | 39 | 55 |
| 76 | 10 | 95 |
| 77 | 150 | 95 |
| 78 | 630 | 88 |
| 79 | 120 | 97 |
| 99 | 201 | 90 |
| 100 | 1000 | 90 |
| 101 | 126 | 95 |

HPK1 protein degradation in human PBMC: Frozen human PBMC were purchased from Shanghai OribioTech and recovered with culture medium (RMPI1640) prior to use. The cells were then incubated with a variety of concentrations of compound. After incubation for 18 h, the cells were collected and lysed. The protein concentration was determined by BCA protein assay kit from Thermo (23227). The HPK1 protein level was determined by western blots, using anti-human HPK1 polyclonal antibody from CST (4472S). Proteins were loaded into each well of the pre-casting gels and subjected to electrophoretic separation by SDS-PAGE. The protein resolved by SDS-PAGE were transferred to PVDF, blocked by 5% skim milk, and probed with anti-human HPK1 antibody or β-actin antibody from CST (3700S), using standard western blotting procedure. The degradation results are shown in the following Table 5.

TABLE 5
Results of HPK1 protein degradation in
human PBMC %

| Example | DC₅₀ (nM) | Dmax (%) |
|---|---|---|
| 16 | 100 | 96 |
| 21 | >500 | 60 |
| 25 | 97 | 96 |
| 26 | 882 | 86 |
| 28 | 97 | 80 |
| 30 | 980 | 61 |
| 31 | 3 | 98 |
| 32 | 100 | 78 |
| 35 | 11 | 90 |
| 36 | 9.1 | 94 |
| 38 | 10 | 30 |
| 39 | 256 | 85 |
| 40 | 220 | 88 |
| 41 | 124 | 90 |
| 42 | 780 | 40 |
| 43 | 110 | 94 |
| 44 | 191 | 30 |
| 45 | 37 | 78 |
| 46 | 164 | 51 |
| 47 | 15 | 85 |
| 48 | 1000 | 57 |
| 49 | >2000 | <50 |
| 51 | 190 | 96 |
| 52 | 875 | 31 |
| 53 | 100 | 86 |
| 54 | 13 | 83 |
| 55 | 440 | 55 |
| 56 | 8.1 | 98 |
| 58 | 2.7 | 98 |
| 63 | 11 | 90 |
| 64 | 15 | 70 |
| 69 | 1200 | 74 |
| 72 | 100 | 30 |
| 73 | 4.8 | 87 |
| 74 | 27 | 86 |
| 76 | 72 | 74 |
| 78 | 30 | 90 |
| 99 | 39 | 99 |
| 100 | 58 | 93 |
| 101 | 93 | 95 |

### 4. Cytokine Measurement

Anti-CD3 Ab was used to provide signal 1 to stimulate mouse CD3+ T cells. Effect of compound stimulation on T cells was measure by IL2 ELISA. Briefly, anti-CD3 Ab was coated onto plates overnight. Isolated T cells from mouse splenocytes were incubated with plate bound CD3 in the presence and absence of compound for 40 hrs. After incubation, the supernatant was collected and IL2 level in the supernatant was determined by mouse IL2 ELISA kit. The results of IL-2 production are shown in following table 6.

TABLE 6
Results of IL-2 production

| Example | Fold change (1 uM) | Fold change (3 uM) |
|---|---|---|
| 36 | 1.8 | 3.8 |
| 58 | 2.5 | 2.0 |
| 63 | 2.1 | 2.0 |
| 73 | 2.0 | <0.5 |
| 79 | 2.6 | <0.5 |
| 88 | 2.3 | 2.4 |
| 95 | 1.8 | 2.7 |
| 101 | 1.7 | 3.7 |
| 102 | 2.9 | 2.6 |
| 103 | 2.1 | 2.8 |

### 5. Results

Examples 36, 58, 63, 73, 79, 88, 95, 101, 102 and 103 were shown to stimulate IL-2 secretion in primary mouse CD3+ T cells.

Examples 1, 16, 21, 31, 35, 51, and 79 were shown to degrade HPK1 protein in primary mouse CD3+ T cells. Examples 31, 58, 63, 73 and 78 were shown to degrade HPK1 protein in human PBMCs. Examples 16, 21, 31, 35, 51, 58, 63, 73, 78, 79 at 1 μM and 10 μM could reduce HPK1 protein expression level compared to DMSO treated counterpart.

### OTHER EMBODIMENTS

The present disclosure provides merely exemplary embodiments. One skilled in the art will readily recognize from the present disclosure and claims, that various changes, modifications and variations can be made therein without departing from the spirit and scope of the present disclosure as defined in the following claims.

1. A compound of Formula (I): 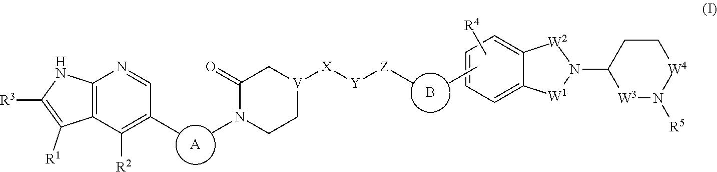
a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) each R², R³ and R⁴ is independently chosen from hydrogen, halogen groups, OR^{x}, SR^{x}, NHR^{x}, N(R^{x})₂, CHR^{x}, and C(R^{x})₂;
(iii) R⁵ is chosen from hydrogen, R^{x}, —CH₂OC(O)R^{x}—, and —CH₂OC(O)C(R^{x}R^{y})NH₂;
(iv) each W¹, W², W³, and W⁴ is independently chosen from C(R^{x})₂ and C(O);
(v) V is chosen from N and CR^{y};
(vi) when V is N, X is absent or is chosen from —C(O)—, —C(O)R^{x}—, —C(S)—, —C(S) R^{x}—, —S(O)₂—, and —S(O)₂R^{x}—; or when V is CR^{x}, X is absent or is chosen from —O—, —S—, —NR^{x}—, —C(O)—, —C(S)—, and —C(R^{x}R^{y})—,
(vii) Y is absent or is chosen from linear, branched, and cyclic alkylene groups and PEG groups;
(viii) Z is absent or is chosen from —O—, —NR^{z}, —NR^{y}C(O)—, C(O)—, —C(S)—, and —C(O)O—;
(ix) each R^{w}, R^{x}, R^{y}, and R^{z} is independently chosen from hydrogen, linear, branched, and cyclic alkyl groups, carbocyclic groups, heterocyclic groups, aryl groups, and heteroaryl groups;
(x) ring A is chosen from aryl groups and heteroaryl groups, and
(xi) ring B is absent or is chosen from aryl groups, heteroaryl groups, cycloalkyl groups, and heterocycloalkyl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

2. A compound of Formula (I′): 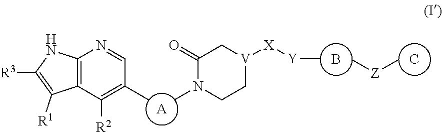
a tautomer thereof, a deuterated derivative of the compound or the tautomer, or a pharmaceutically acceptable salt the foregoing, wherein:
(i) R¹ is chosen from linear, branched, and cyclic alkyl groups, carbocyclic groups, heterocyclic groups, linear, branched, and cyclic alkenyl groups, linear and branched heteroalkenyl groups, linear, branched, and cyclic alkynyl groups, CO₂R^{x}, C(O)NR^{x}R^{y}, C(O)R^{x}OR^{y}, C(O)R^{w}N(R^{x}R^{y})₂, OC(O)R^{w}NR^{x}R^{y}, S(O)R^{y}, and SO₂R^{y};
(ii) each R² and R³ is independently chosen from hydrogen, halogen groups, OR^{x}, SR^{x}, NHR^{x}, N(R^{x})₂, CHR^{x}, and C(R^{x})₂;
(iii) V is chosen from N and CR^{x};
(iv) when V is N, X is absent or is chosen from —C(O)—, —C(O)R^{x}—, —C(S)—, —C(S) R^{x}, —S(O)₂—, and —S(O)₂R^{x}—; or when V is CR^{x}, X is absent or is chosen from —O—, —S—, —NR^{x}—, —C(O)—, —C(S)—, and —C(R^{x}R^{y}),
(v) Y is absent or is chosen from linear, branched, and cyclic alkylene groups and PEG groups;
(vi) Z is absent or is chosen from —O—, —NR^{z}—, —NR^{y}C(O)—, —C(O)—, —C(S)—, and —C(O)O—;
(vii) each R^{w}, R^{x}, R^{y}, and R^{z} is independently chosen from hydrogen, linear, branched, and cyclic alkyl groups, carbocyclic groups, heterocyclic groups, aryl groups, and heteroaryl groups;
(viii) ring A is chosen from aryl groups and heteroaryl groups, and
(ix) ring B′ is absent or is chosen from aryl groups, heteroaryl groups, cycloalkyl groups, and heterocycloalkyl groups;
(x) ring C is chosen from 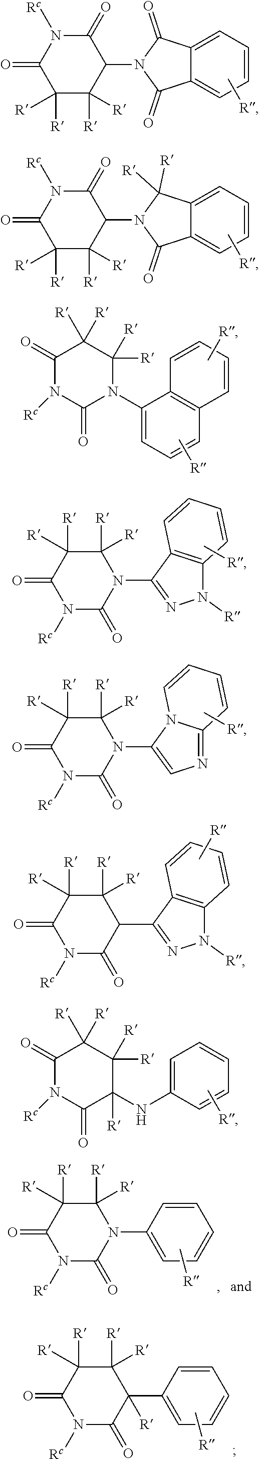
wherein R^{c} is chosen from hydrogen, linear, branched, and cyclic alkyl groups;
each R′ and R″ is chosen from hydrogen, halogen groups, OR^{x}, linear, branched, and cyclic alkyl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

3. The compound, tautomer, deuterated derivative, or pharmaceutically acceptable salt of any of claims 1-2, wherein Y is chosen from PEG groups.

4. The compound, tautomer, deuterated derivative, or pharmaceutically acceptable salt of claim 36, wherein ring B is chosen from

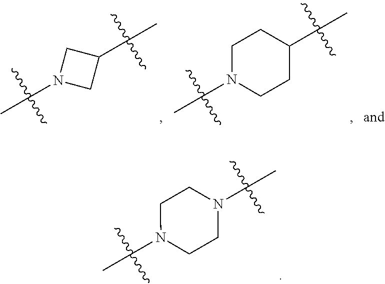 [SMILES: *C1CN(*)C1 ; *C1CCN(*)CC1 ; *N1CCN(*)CC1]

5. A compound chosen from 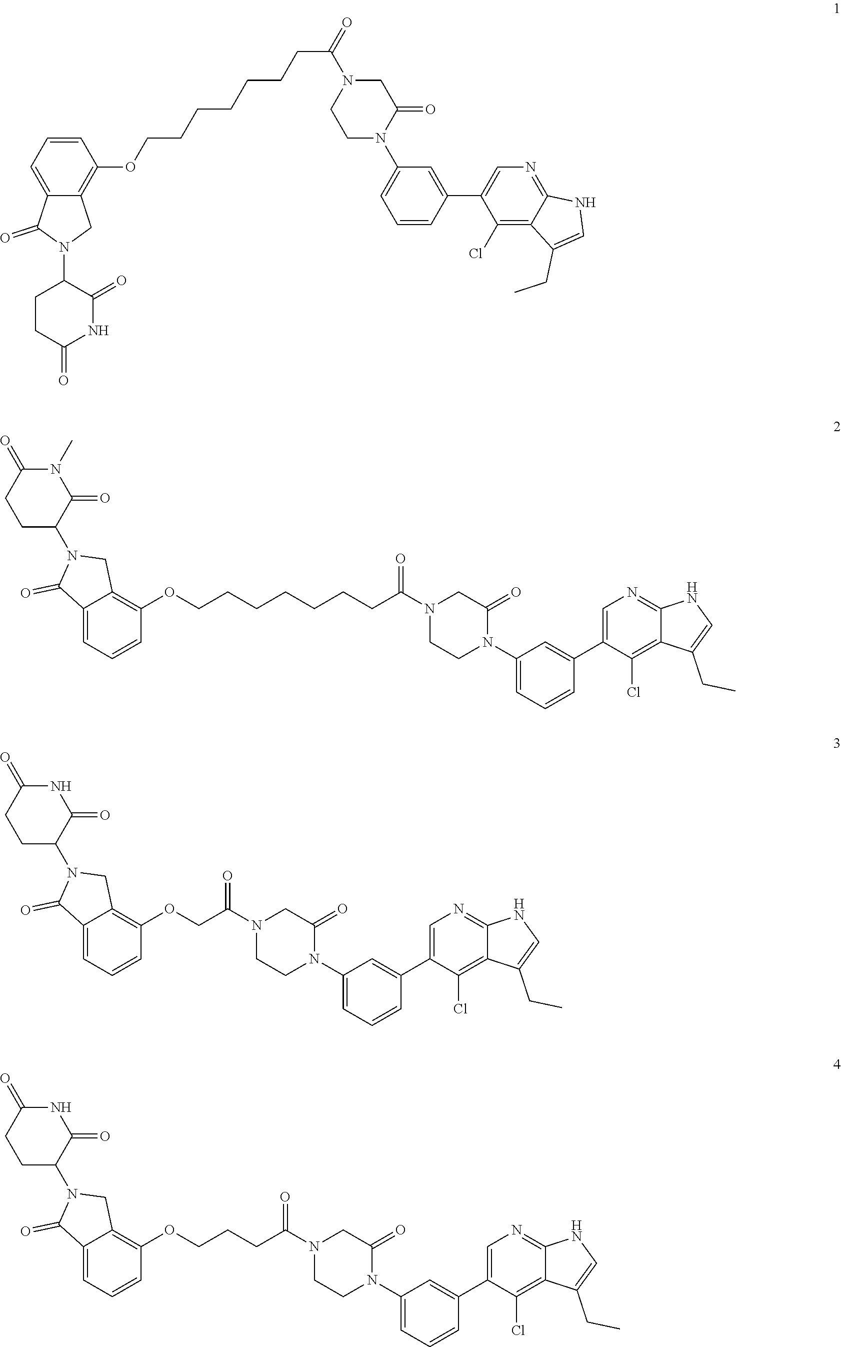 [SMILES: CCc1c[nH]c2ncc(-c3cccc(N4CCN(C(=O)CCCCCCCOc5cccc6c5CN(C5CCC(=O)NC5=O)C6=O)CC4=O)c3)c(Cl)c12 ; CCc1c[nH]c2ncc(-c3cccc(N4CCN(C(=O)CCCCCCCOc5cccc6c5CN(C5CCC(=O)N(C)C5=O)C6=O)CC4=O)c3)c(Cl)c12 ; CCc1c[nH]c2ncc(-c3cccc(N4CCN(C(=O)COc5cccc6c5CN(C5CCC(=O)NC5=O)C6=O)CC4=O)c3)c(Cl)c12 ; CCc1c[nH]c2ncc(-c3cccc(N4CCN(C(=O)CCCOc5cccc6c5CN(C5CCC(=O)NC5=O)C6=O)CC4=O)c3)c(Cl)c12] 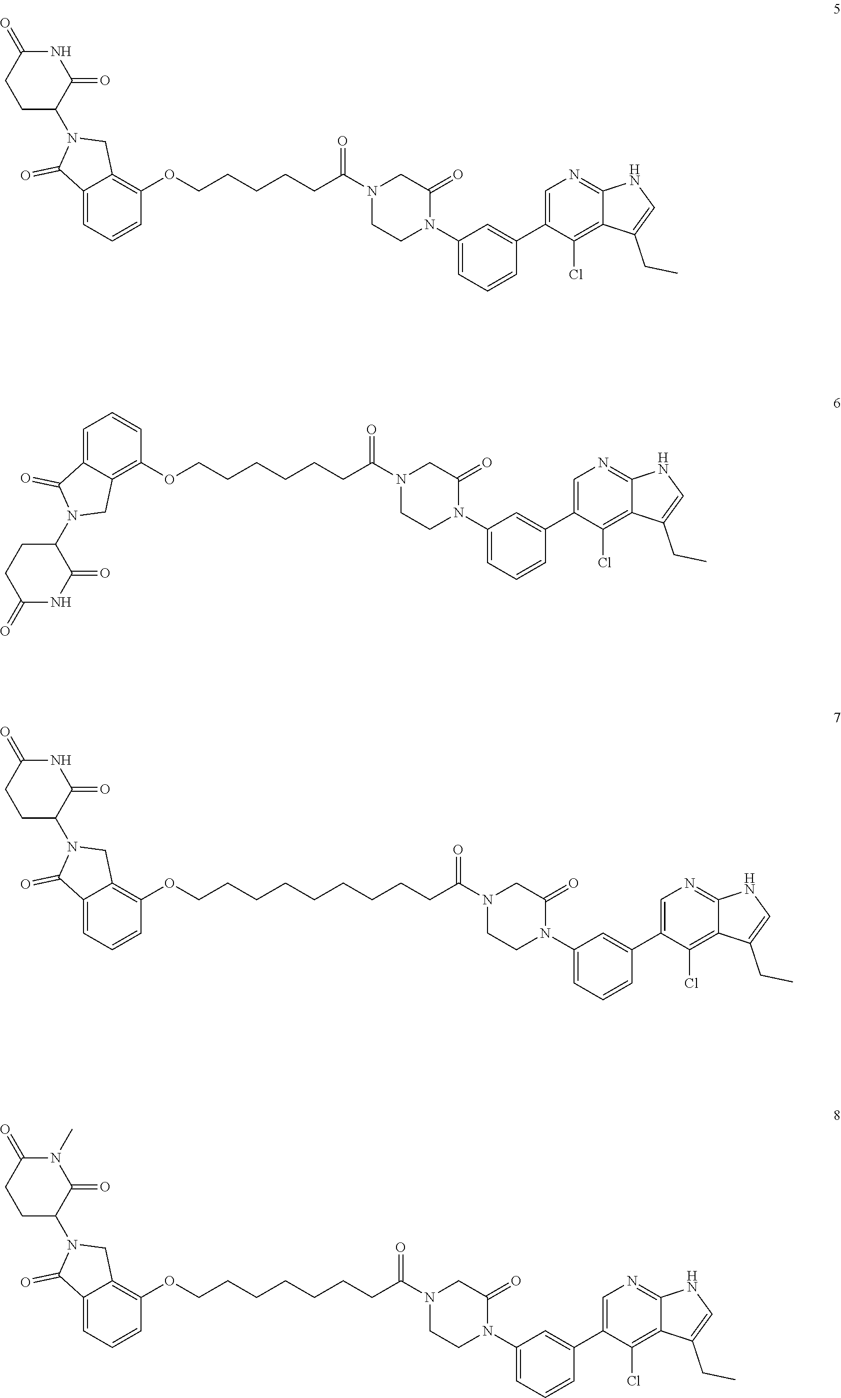 [SMILES: CCc1c[nH]c2ncc(-c3cccc(N4CCN(C(=O)CCCCCOc5cccc6c5CN(C5CCC(=O)NC5=O)C6=O)CC4=O)c3)c(Cl)c12 ; CCc1c[nH]c2ncc(-c3cccc(N4CCN(C(=O)CCCCCCOc5cccc6c5CN(C5CCC(=O)NC5=O)C6=O)CC4=O)c3)c(Cl)c12 ; CCc1c[nH]c2ncc(-c3cccc(N4CCN(C(=O)CCCCCCCCCOc5cccc6c5CN(C5CCC(=O)NC5=O)C6=O)CC4=O)c3)c(Cl)c12 ; CCc1c[nH]c2ncc(-c3cccc(N4CCN(C(=O)CCCCCCCOc5cccc6c5CN(C5CCC(=O)N(C)C5=O)C6=O)CC4=O)c3)c(Cl)c12] 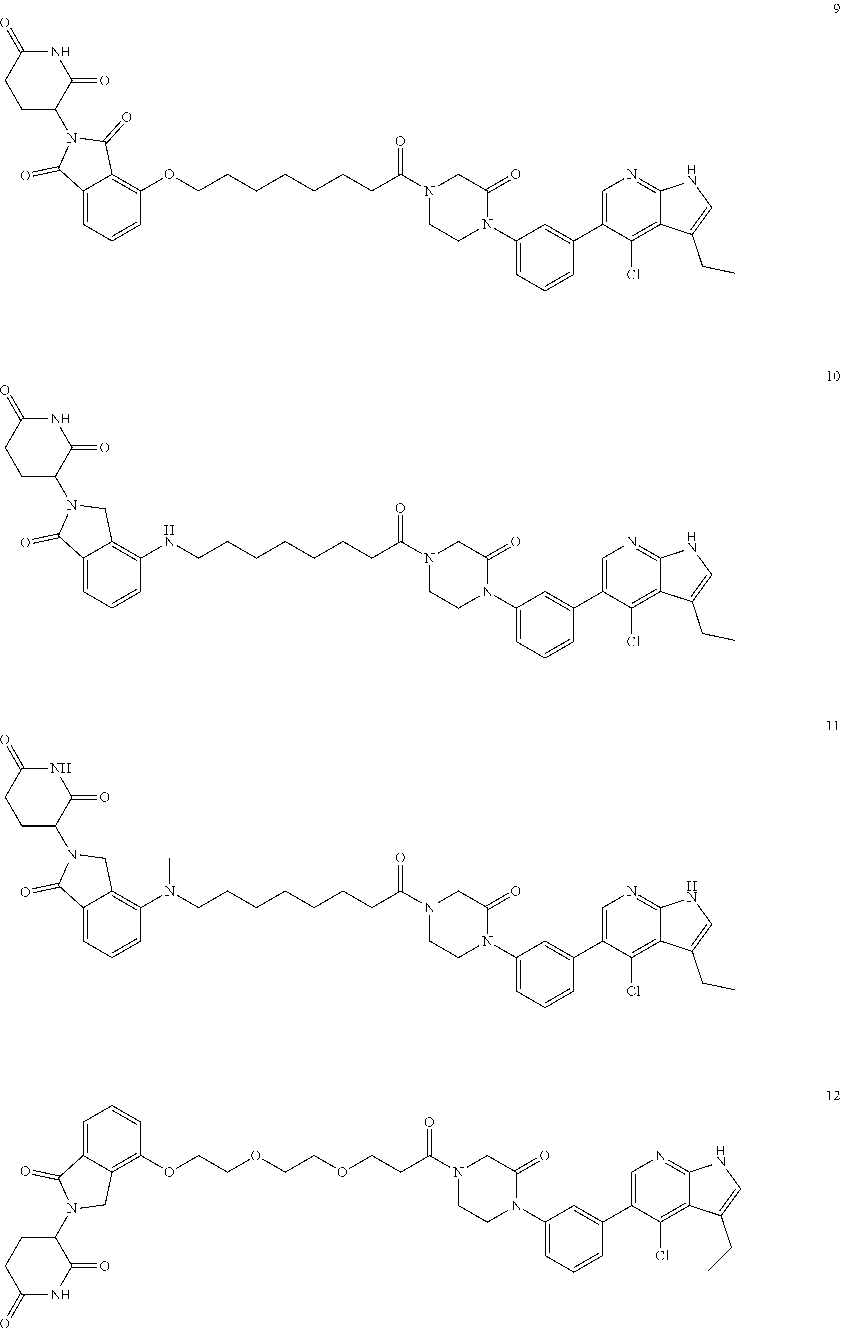 [SMILES: CCc1c[nH]c2ncc(-c3cccc(N4CCN(C(=O)CCCCCCCOc5cccc6c5C(=O)N(C5CCC(=O)NC5=O)C6=O)CC4=O)c3)c(Cl)c12 ; CCc1c[nH]c2ncc(-c3cccc(N4CCN(C(=O)CCCCCCCNc5cccc6c5CN(C5CCC(=O)NC5=O)C6=O)CC4=O)c3)c(Cl)c12 ; CCc1c[nH]c2ncc(-c3cccc(N4CCN(C(=O)CCCCCCCN(C)c5cccc6c5CN(C5CCC(=O)NC5=O)C6=O)CC4=O)c3)c(Cl)c12 ; CCc1c[nH]c2ncc(-c3cccc(N4CCN(C(=O)CCOCCOCCOc5cccc6c5CN(C5CCC(=O)NC5=O)C6=O)CC4=O)c3)c(Cl)c12] 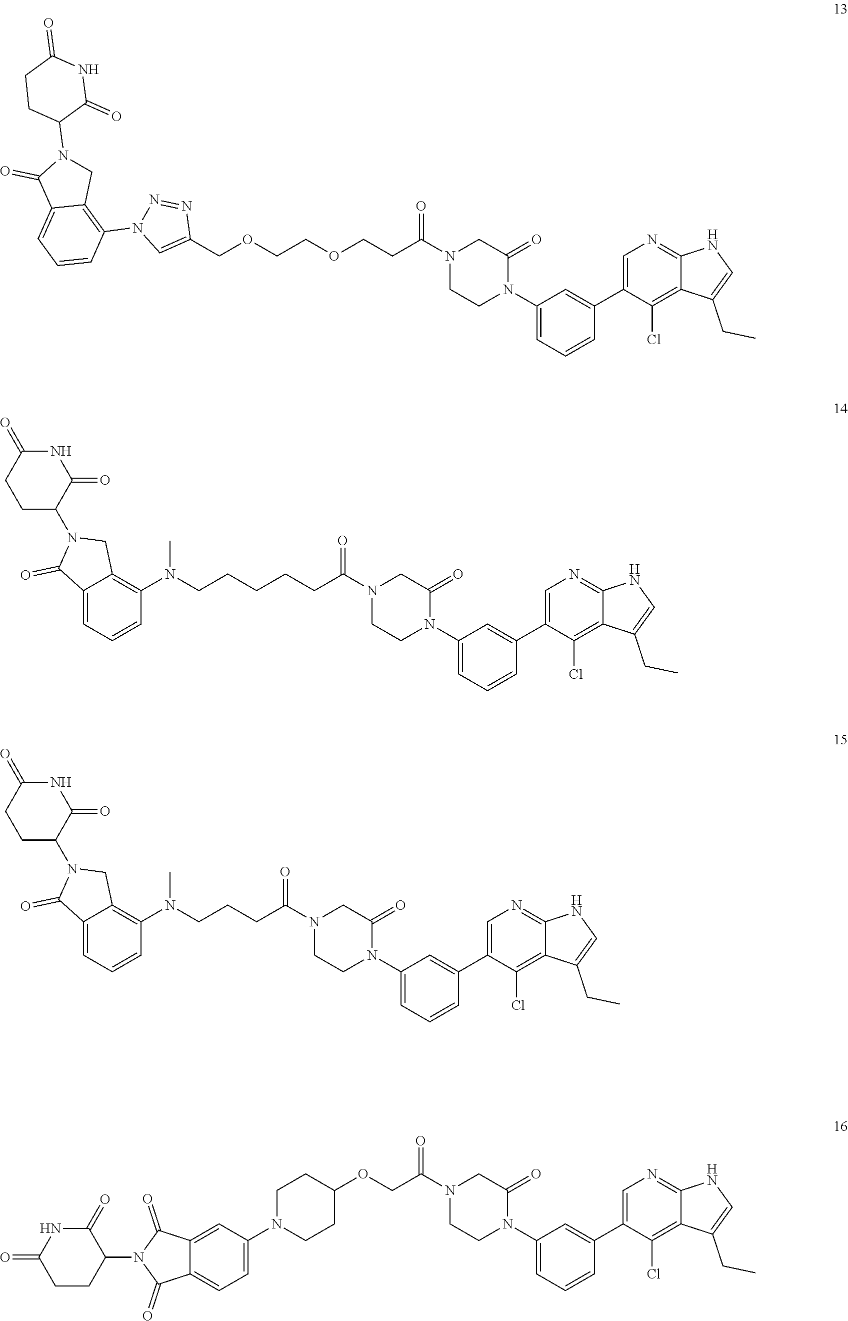 [SMILES: CCc1c[nH]c2ncc(-c3cccc(N4CCN(C(=O)CCOCCOCc5cn(-c6cccc7c6CN(C6CCC(=O)NC6=O)C7=O)nn5)CC4=O)c3)c(Cl)c12 ; CCc1c[nH]c2ncc(-c3cccc(N4CCN(C(=O)CCCCCN(C)c5cccc6c5CN(C5CCC(=O)NC5=O)C6=O)CC4=O)c3)c(Cl)c12 ; CCc1c[nH]c2ncc(-c3cccc(N4CCN(C(=O)CCCN(C)c5cccc6c5CN(C5CCC(=O)NC5=O)C6=O)CC4=O)c3)c(Cl)c12 ; CCc1c[nH]c2ncc(-c3cccc(N4CCN(C(=O)COC5CCN(c6ccc7c(c6)C(=O)N(C6CCC(=O)NC6=O)C7=O)CC5)CC4=O)c3)c(Cl)c12] 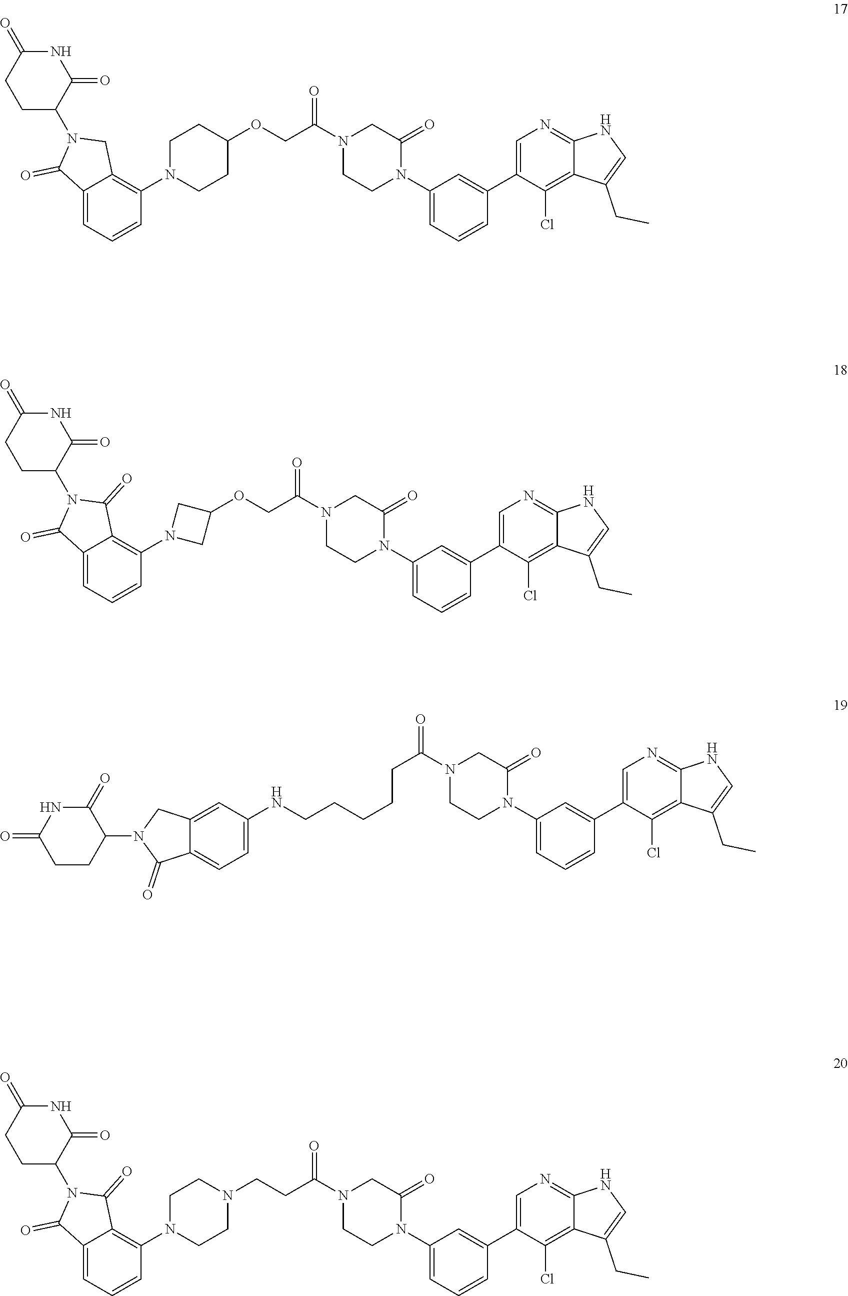 [SMILES: CCc1c[nH]c2ncc(-c3cccc(N4CCN(C(=O)COC5CCN(c6cccc7c6CN(C6CCC(=O)NC6=O)C7=O)CC5)CC4=O)c3)c(Cl)c12 ; CCc1c[nH]c2ncc(-c3cccc(N4CCN(C(=O)COC5CN(c6cccc7c6C(=O)N(C6CCC(=O)NC6=O)C7=O)C5)CC4=O)c3)c(Cl)c12 ; CCc1c[nH]c2ncc(-c3cccc(N4CCN(C(=O)CCCCCNc5ccc6c(c5)CN(C5CCC(=O)NC5=O)C6=O)CC4=O)c3)c(Cl)c12 ; CCc1c[nH]c2ncc(-c3cccc(N4CCN(C(=O)CCN5CCN(c6cccc7c6C(=O)N(C6CCC(=O)NC6=O)C7=O)CC5)CC4=O)c3)c(Cl)c12] 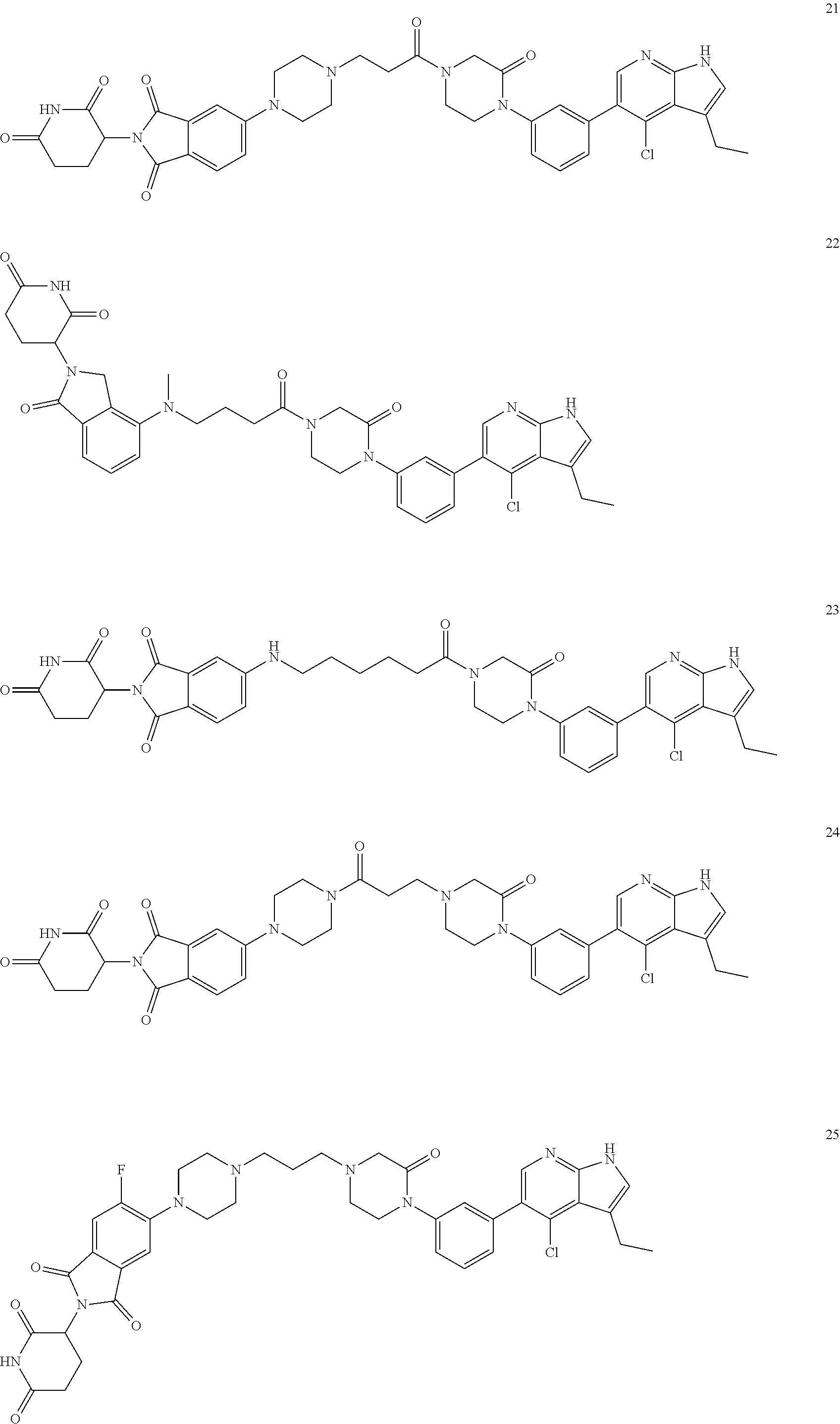 [SMILES: CCc1c[nH]c2ncc(-c3cccc(N4CCN(C(=O)CCN5CCN(c6ccc7c(c6)C(=O)N(C6CCC(=O)NC6=O)C7=O)CC5)CC4=O)c3)c(Cl)c12 ; CCc1c[nH]c2ncc(-c3cccc(N4CCN(C(=O)CCCN(C)c5cccc6c5CN(C5CCC(=O)NC5=O)C6=O)CC4=O)c3)c(Cl)c12 ; CCc1c[nH]c2ncc(-c3cccc(N4CCN(C(=O)CCCCCNc5ccc6c(c5)C(=O)N(C5CCC(=O)NC5=O)C6=O)CC4=O)c3)c(Cl)c12 ; CCc1c[nH]c2ncc(-c3cccc(N4CCN(CCC(=O)N5CCN(c6ccc7c(c6)C(=O)N(C6CCC(=O)NC6=O)C7=O)CC5)CC4=O)c3)c(Cl)c12 ; CCc1c[nH]c2ncc(-c3cccc(N4CCN(CCCN5CCN(c6cc7c(cc6F)C(=O)N(C6CCC(=O)NC6=O)C7=O)CC5)CC4=O)c3)c(Cl)c12] 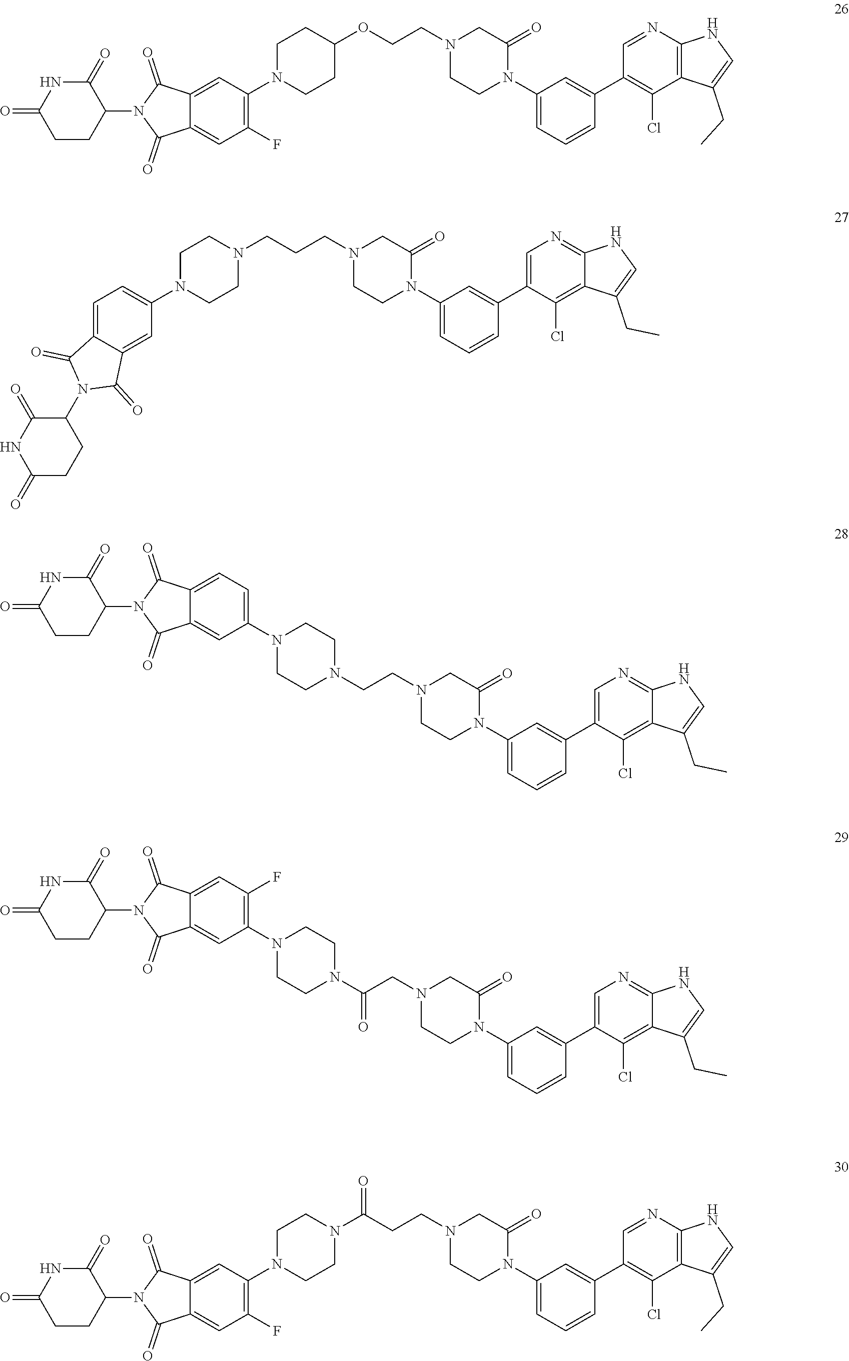 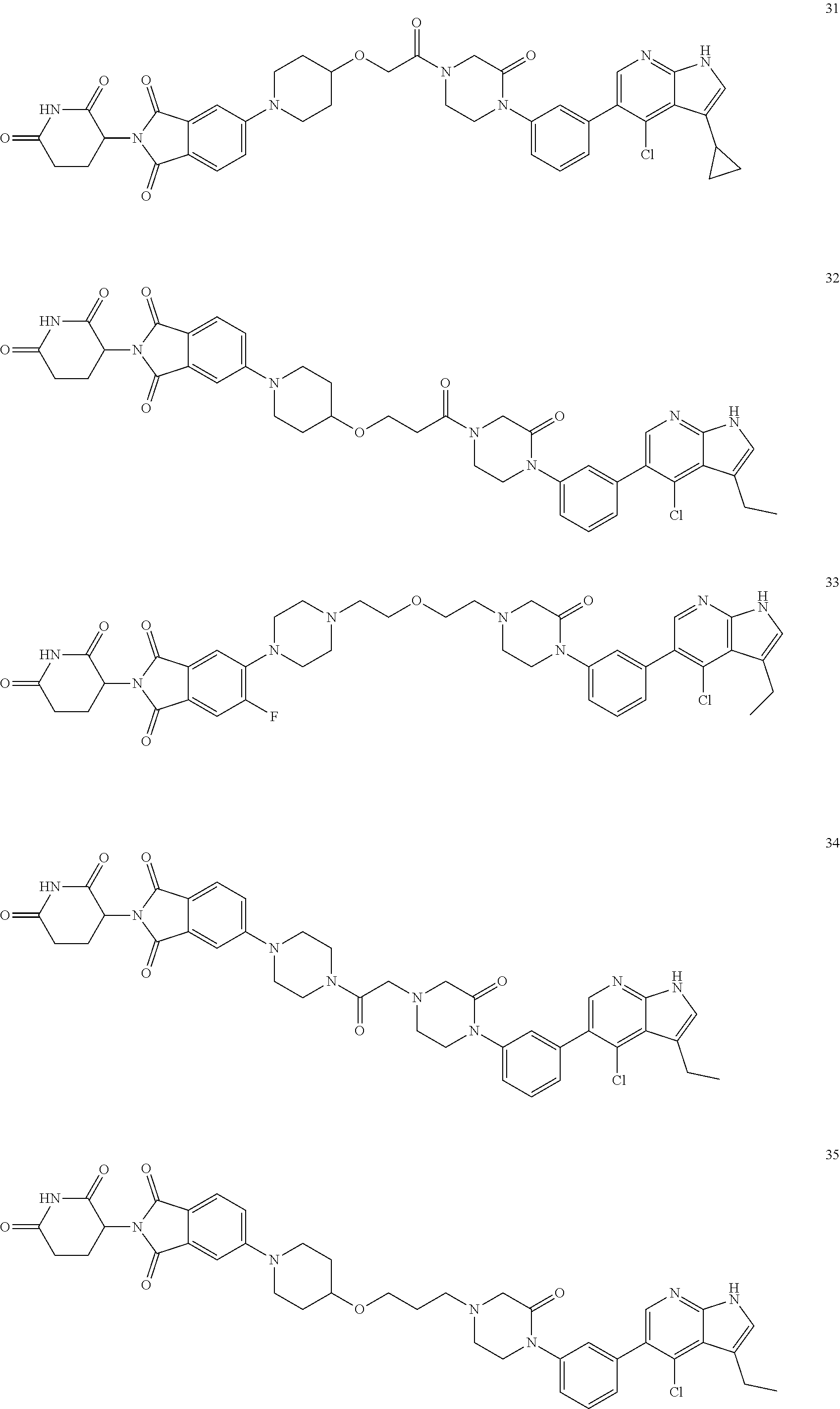 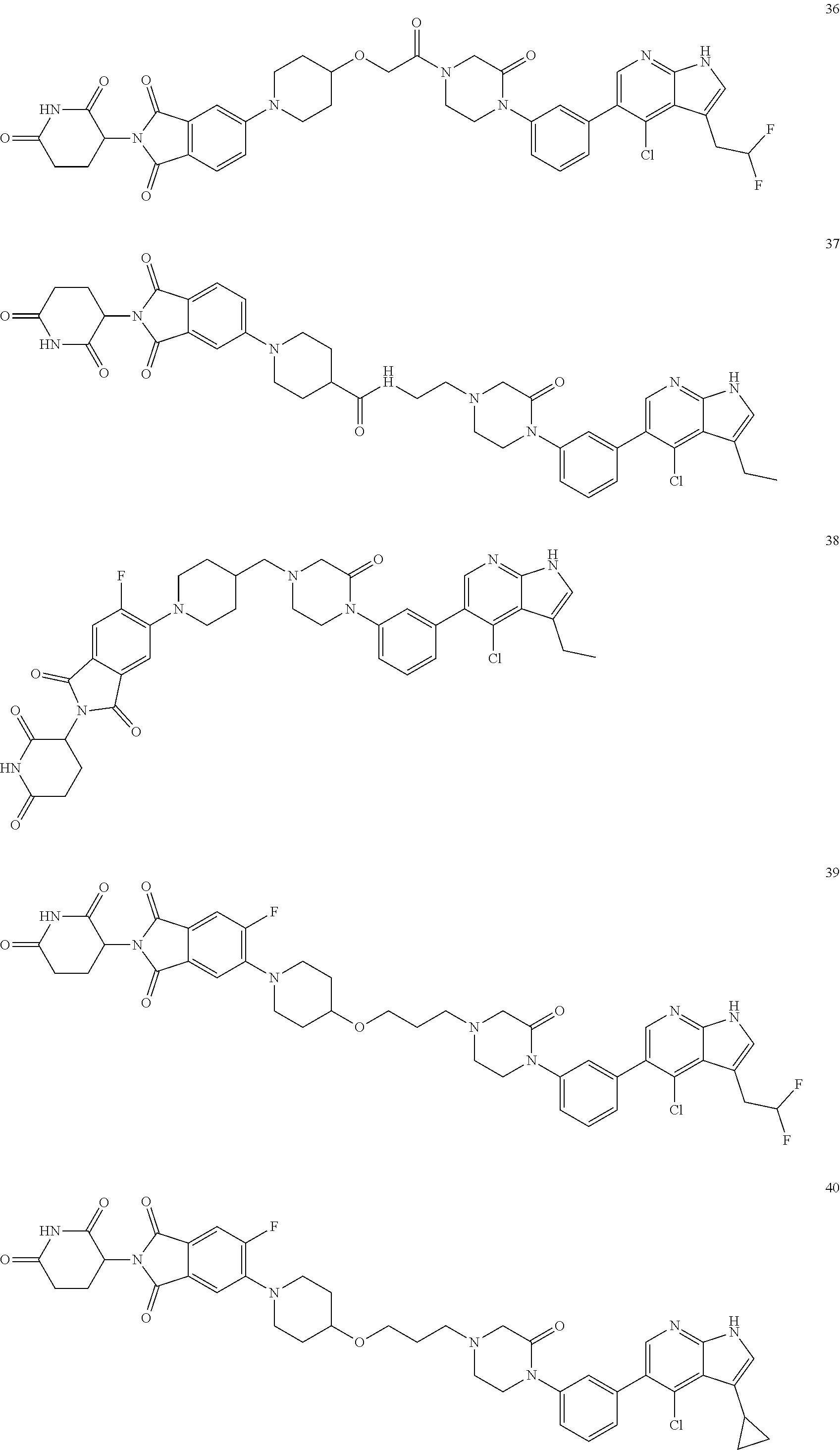 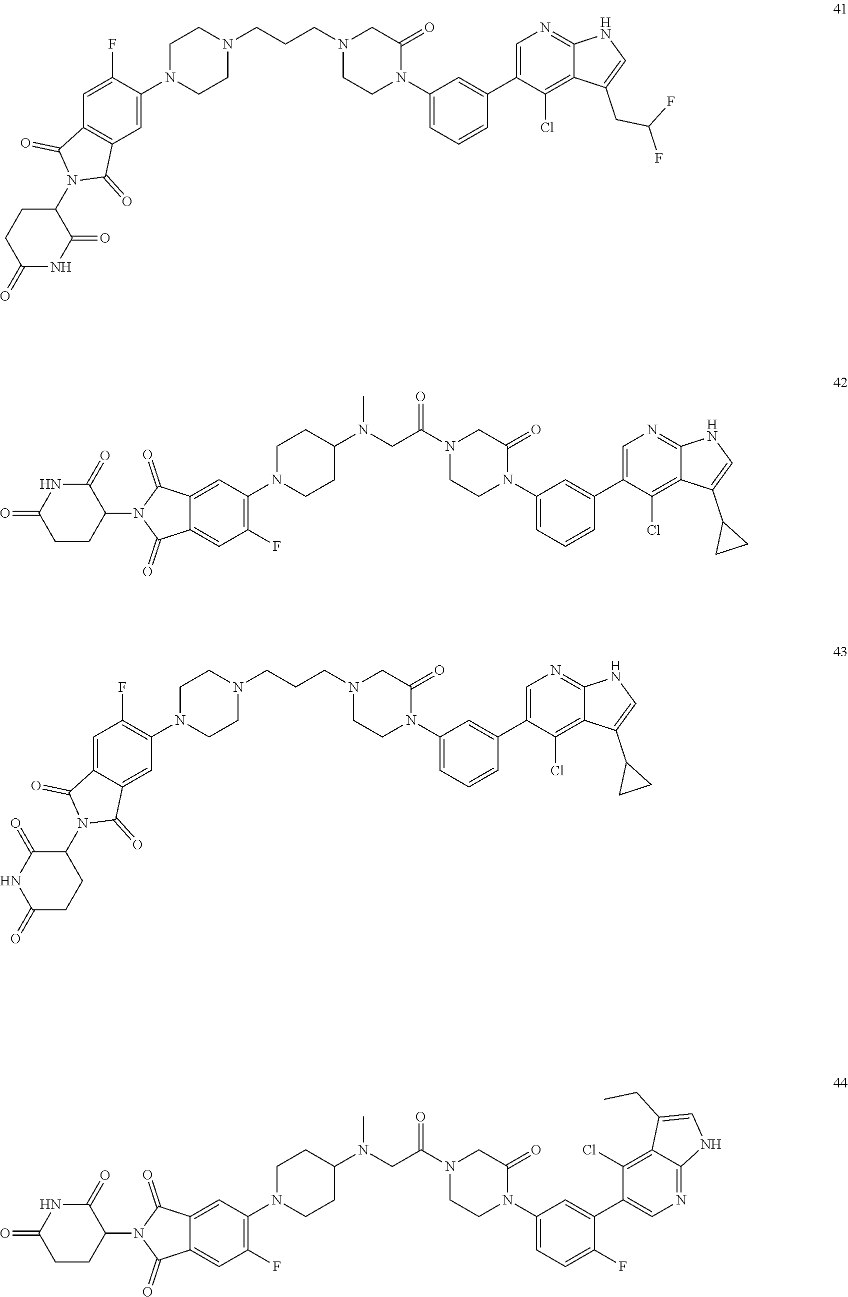 [SMILES: O=C1CCC(N2C(=O)c3cc(F)c(N4CCN(CCCN5CCN(c6cccc(-c7cnc8[nH]cc(CC(F)F)c8c7Cl)c6)C(=O)C5)CC4)cc3C2=O)C(=O)N1 ; CN(CC(=O)N1CCN(c2cccc(-c3cnc4[nH]cc(C5CC5)c4c3Cl)c2)C(=O)C1)C1CCN(c2cc3c(cc2F)C(=O)N(C2CCC(=O)NC2=O)C3=O)CC1 ; O=C1CCC(N2C(=O)c3cc(F)c(N4CCN(CCCN5CCN(c6cccc(-c7cnc8[nH]cc(C9CC9)c8c7Cl)c6)C(=O)C5)CC4)cc3C2=O)C(=O)N1 ; CCc1c[nH]c2ncc(-c3cc(N4CCN(C(=O)CN(C)C5CCN(c6cc7c(cc6F)C(=O)N(C6CCC(=O)NC6=O)C7=O)CC5)CC4=O)ccc3F)c(Cl)c12] 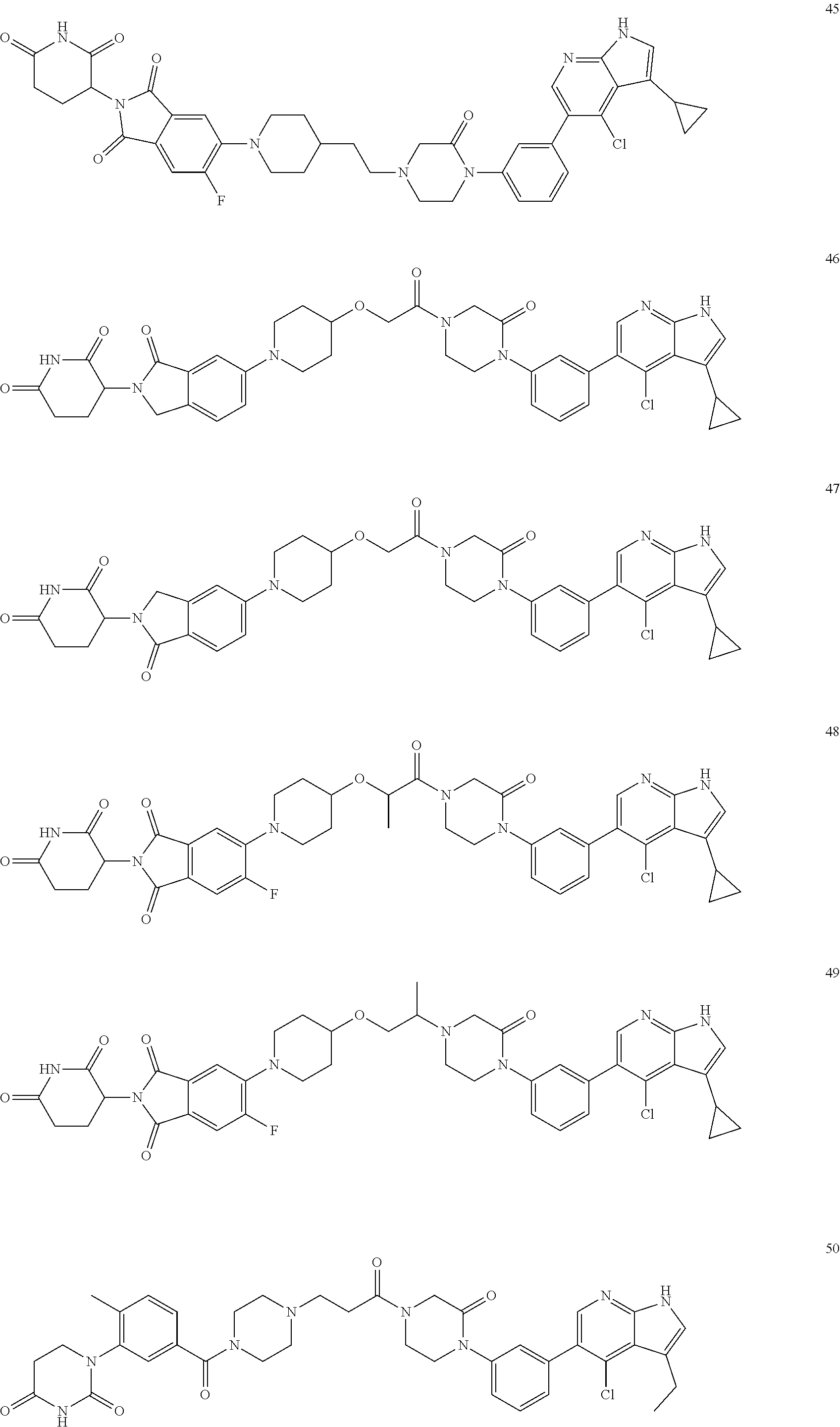 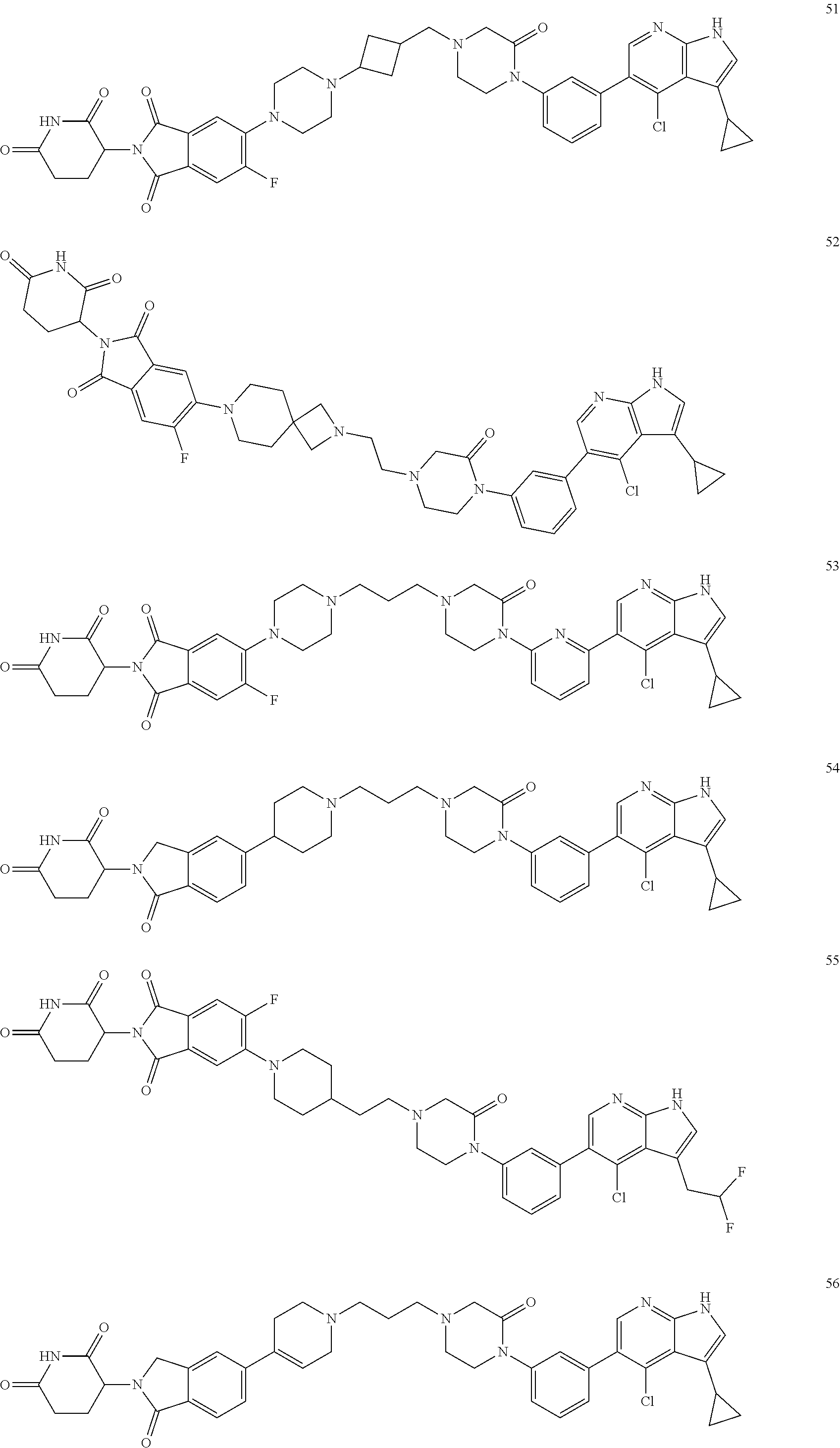 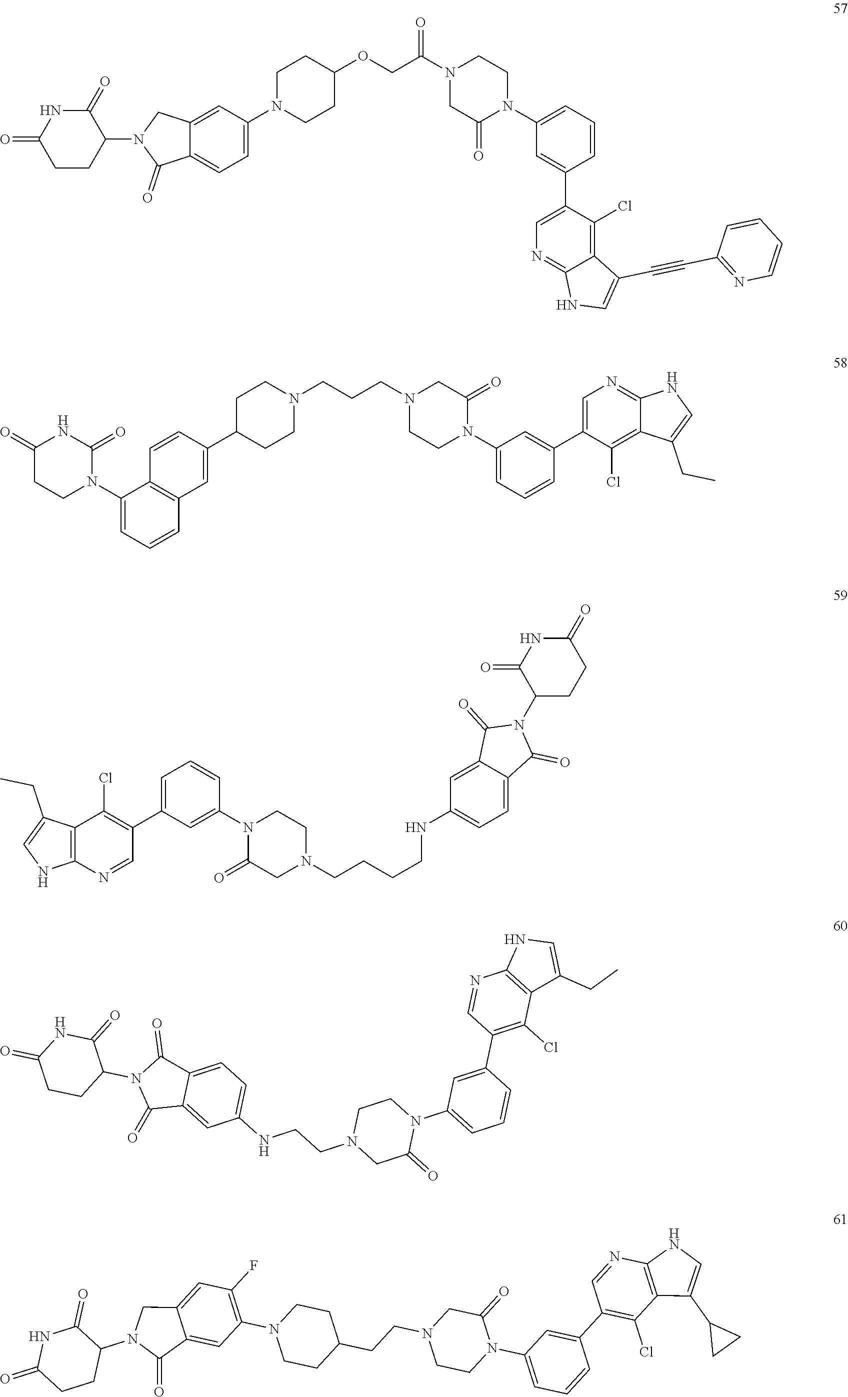 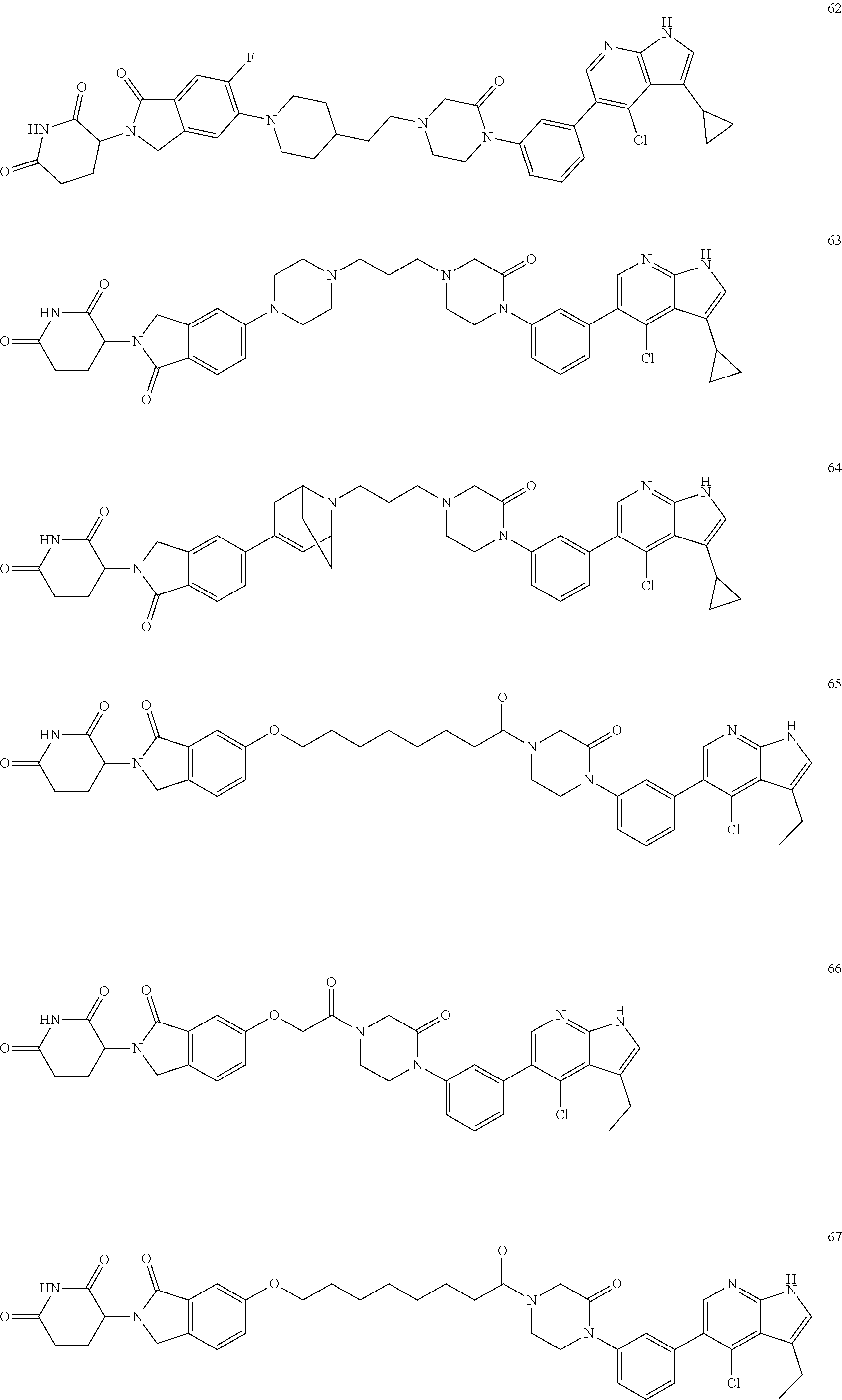 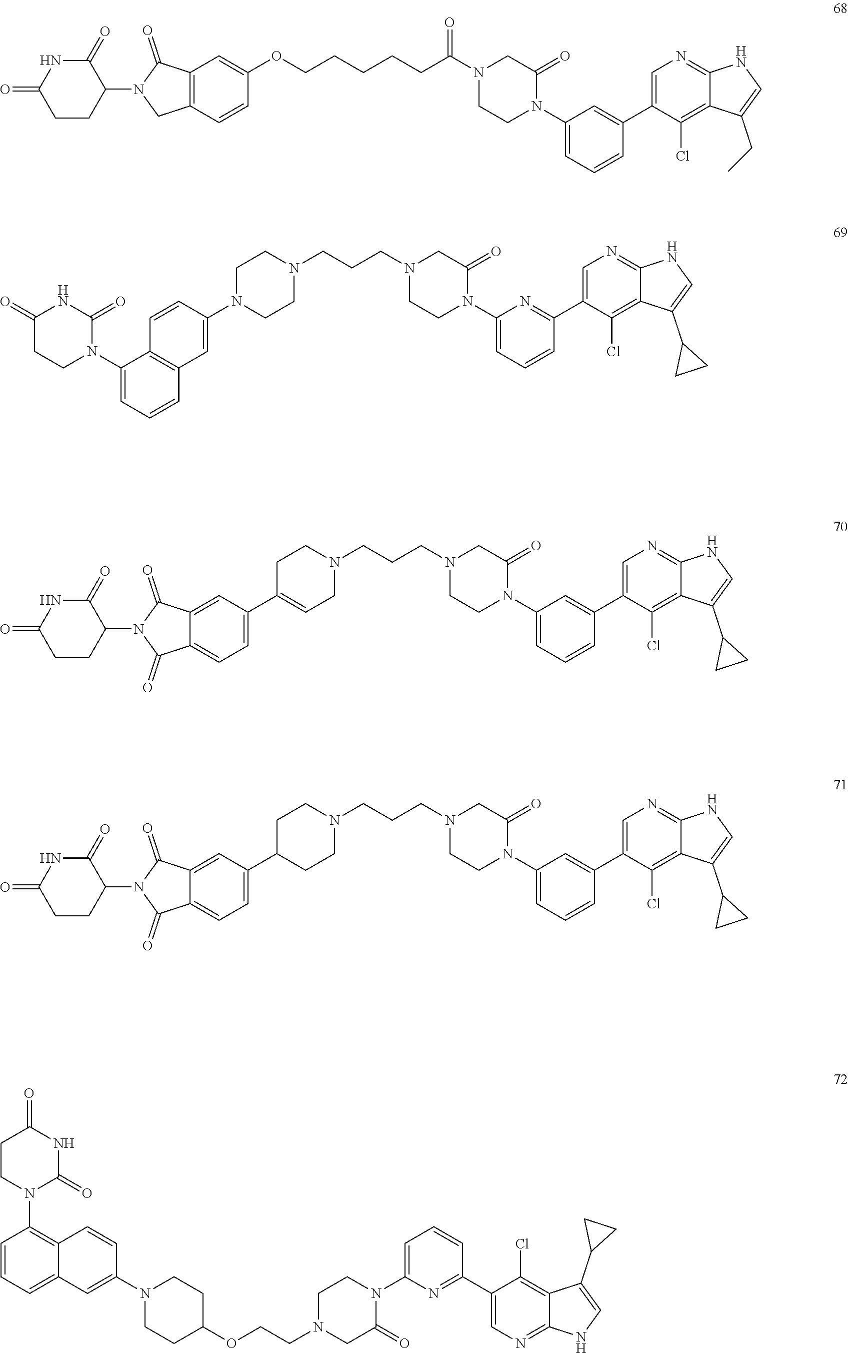 [SMILES: CCc1c[nH]c2ncc(-c3cccc(N4CCN(C(=O)CCCCCOc5ccc6c(c5)C(=O)N(C5CCC(=O)NC5=O)C6)CC4=O)c3)c(Cl)c12 ; O=C1CCN(c2cccc3cc(N4CCN(CCCN5CCN(c6cccc(-c7cnc8[nH]cc(C9CC9)c8c7Cl)n6)C(=O)C5)CC4)ccc23)C(=O)N1 ; O=C1CCC(N2C(=O)c3ccc(C4=CCN(CCCN5CCN(c6cccc(-c7cnc8[nH]cc(C9CC9)c8c7Cl)c6)C(=O)C5)CC4)cc3C2=O)C(=O)N1 ; O=C1CCC(N2C(=O)c3ccc(C4CCN(CCCN5CCN(c6cccc(-c7cnc8[nH]cc(C9CC9)c8c7Cl)c6)C(=O)C5)CC4)cc3C2=O)C(=O)N1 ; O=C1CCN(c2cccc3cc(N4CCC(OCCN5CCN(c6cccc(-c7cnc8[nH]cc(C9CC9)c8c7Cl)n6)C(=O)C5)CC4)ccc23)C(=O)N1] 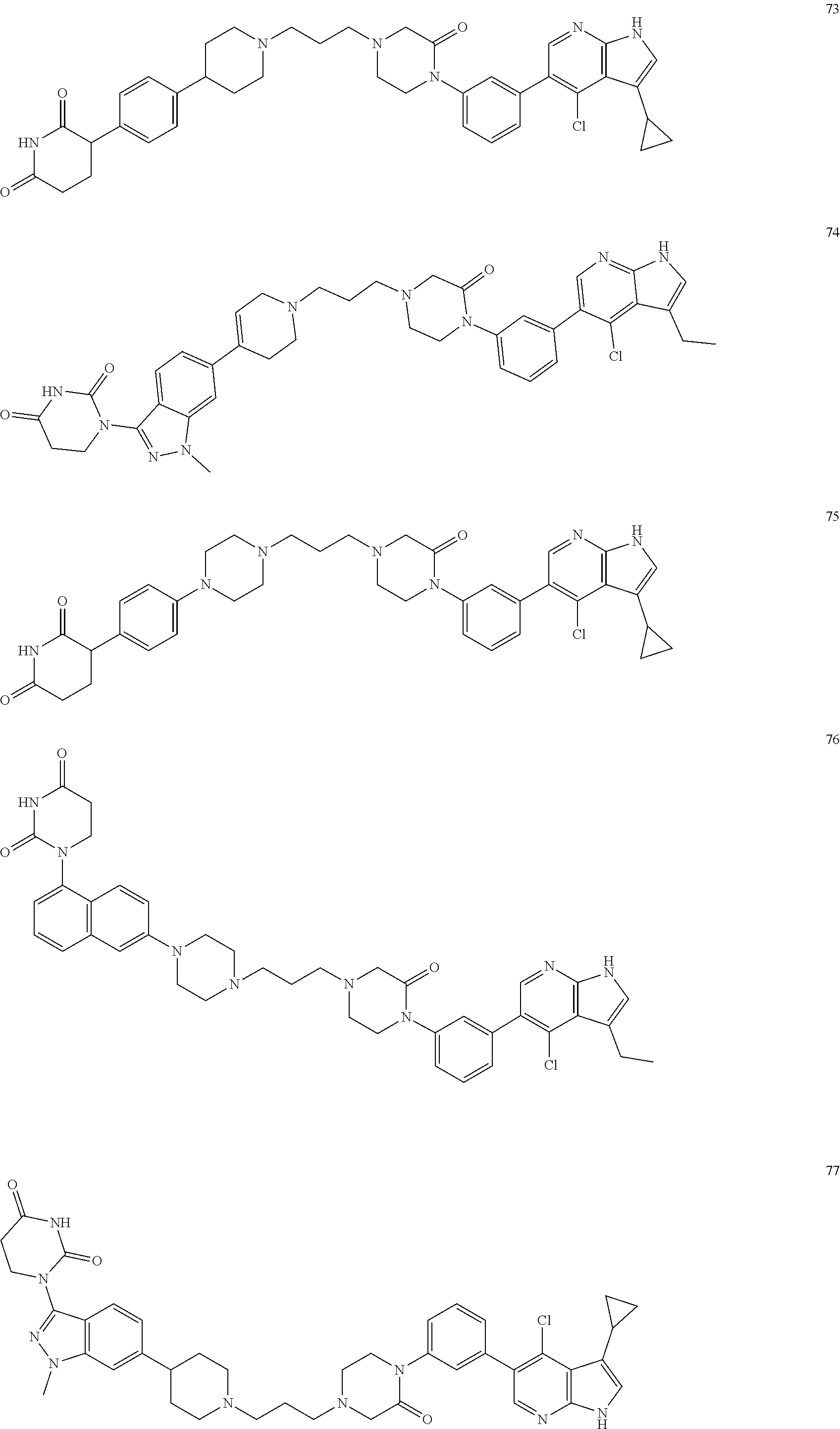 [SMILES: O=C1CCC(c2ccc(C3CCN(CCCN4CCN(c5cccc(-c6cnc7[nH]cc(C8CC8)c7c6Cl)c5)C(=O)C4)CC3)cc2)C(=O)N1 ; CCc1c[nH]c2ncc(-c3cccc(N4CCN(CCCN5CC=C(c6ccc7c(N8CCC(=O)NC8=O)nn(C)c7c6)CC5)CC4=O)c3)c(Cl)c12 ; O=C1CCC(c2ccc(N3CCN(CCCN4CCN(c5cccc(-c6cnc7[nH]cc(C8CC8)c7c6Cl)c5)C(=O)C4)CC3)cc2)C(=O)N1 ; CCc1c[nH]c2ncc(-c3cccc(N4CCN(CCCN5CCN(c6ccc7c(N8CCC(=O)NC8=O)cccc7c6)CC5)CC4=O)c3)c(Cl)c12 ; Cn1nc(N2CCC(=O)NC2=O)c2ccc(C3CCN(CCCN4CCN(c5cccc(-c6cnc7[nH]cc(C8CC8)c7c6Cl)c5)C(=O)C4)CC3)cc21] 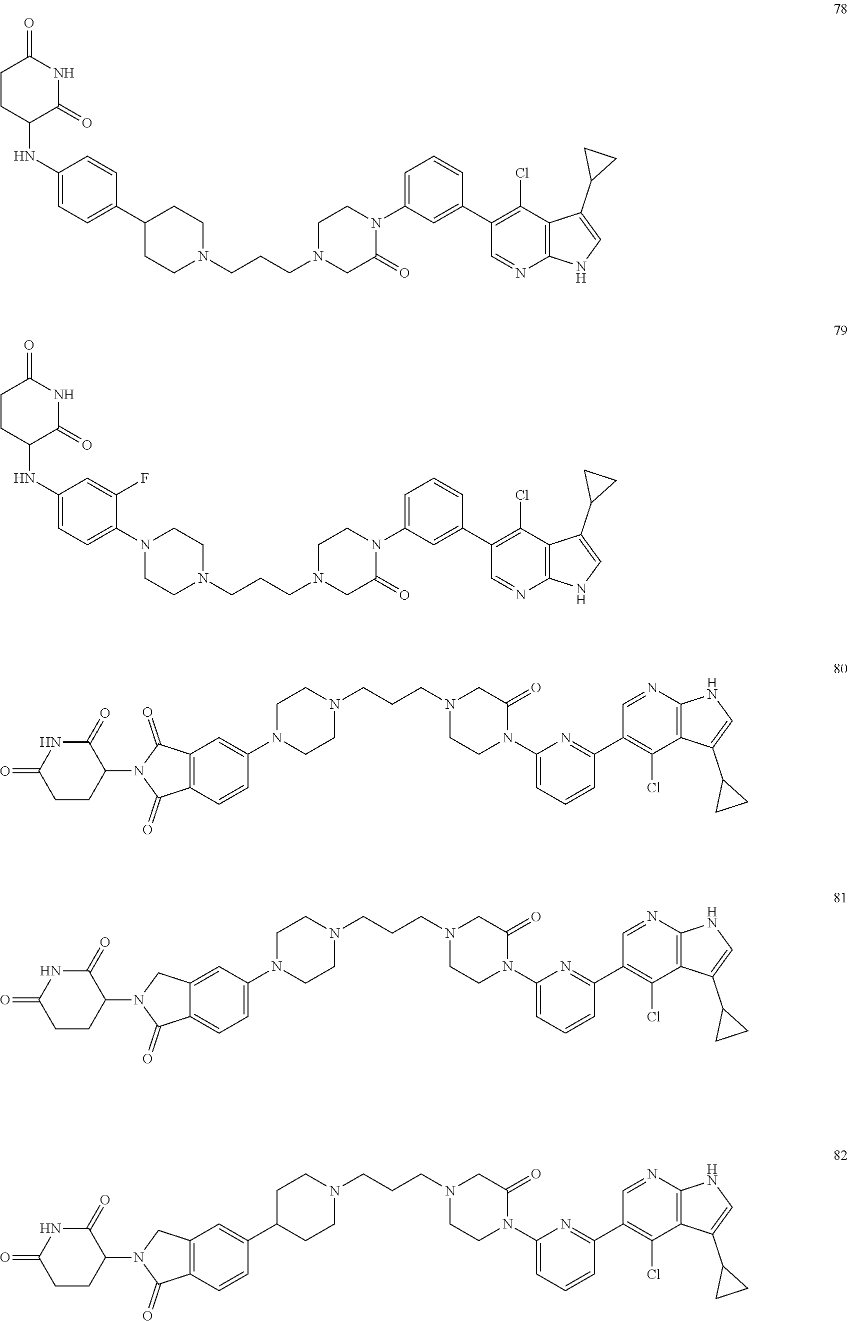 [SMILES: O=C1CCC(Nc2ccc(C3CCN(CCCN4CCN(c5cccc(-c6cnc7[nH]cc(C8CC8)c7c6Cl)c5)C(=O)C4)CC3)cc2)C(=O)N1 ; O=C1CCC(Nc2ccc(N3CCN(CCCN4CCN(c5cccc(-c6cnc7[nH]cc(C8CC8)c7c6Cl)c5)C(=O)C4)CC3)c(F)c2)C(=O)N1 ; O=C1CCC(N2C(=O)c3ccc(N4CCN(CCCN5CCN(c6cccc(-c7cnc8[nH]cc(C9CC9)c8c7Cl)n6)C(=O)C5)CC4)cc3C2=O)C(=O)N1 ; O=C1CCC(N2Cc3cc(N4CCN(CCCN5CCN(c6cccc(-c7cnc8[nH]cc(C9CC9)c8c7Cl)n6)C(=O)C5)CC4)ccc3C2=O)C(=O)N1 ; O=C1CCC(N2Cc3cc(C4CCN(CCCN5CCN(c6cccc(-c7cnc8[nH]cc(C9CC9)c8c7Cl)n6)C(=O)C5)CC4)ccc3C2=O)C(=O)N1] 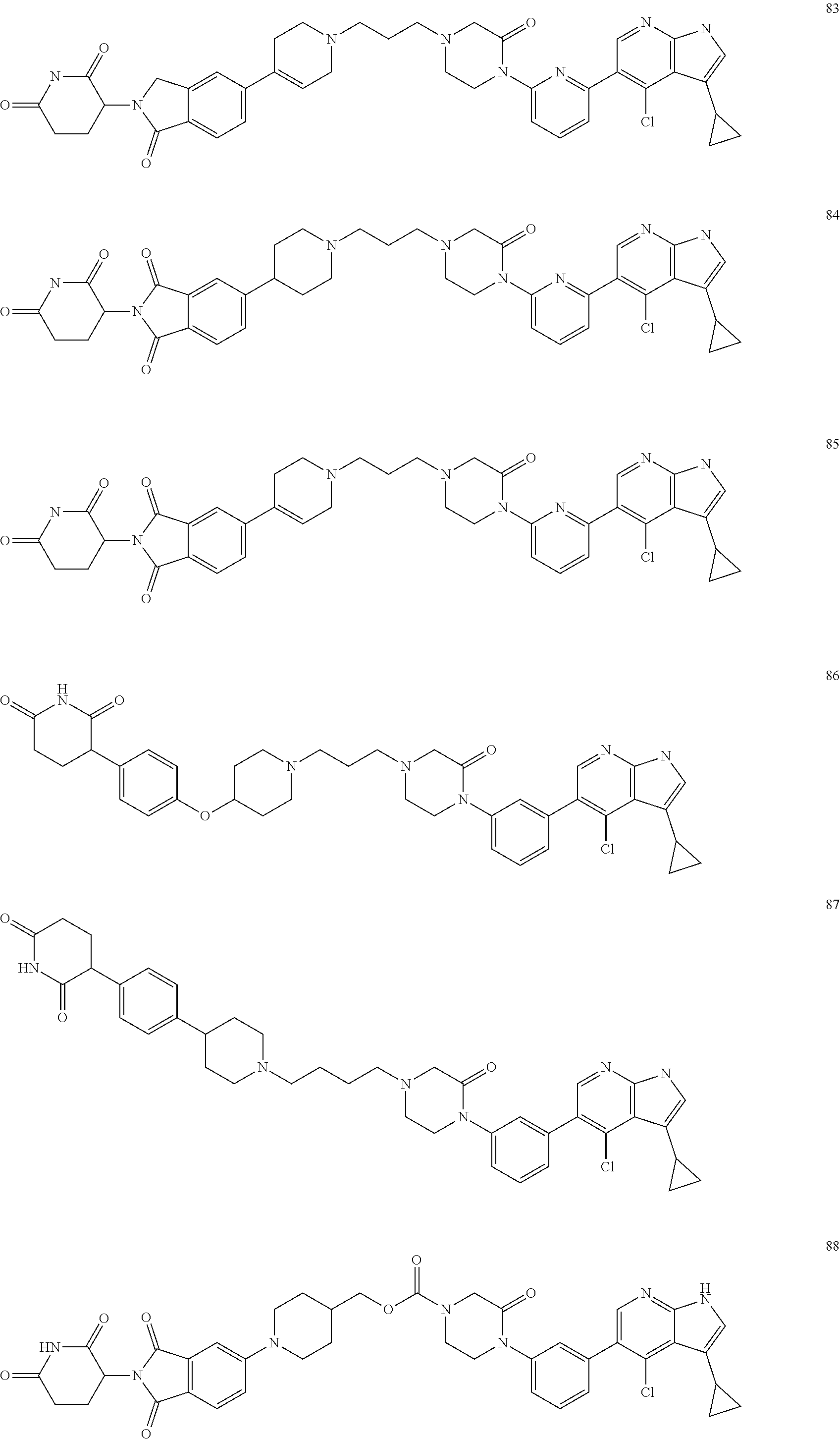 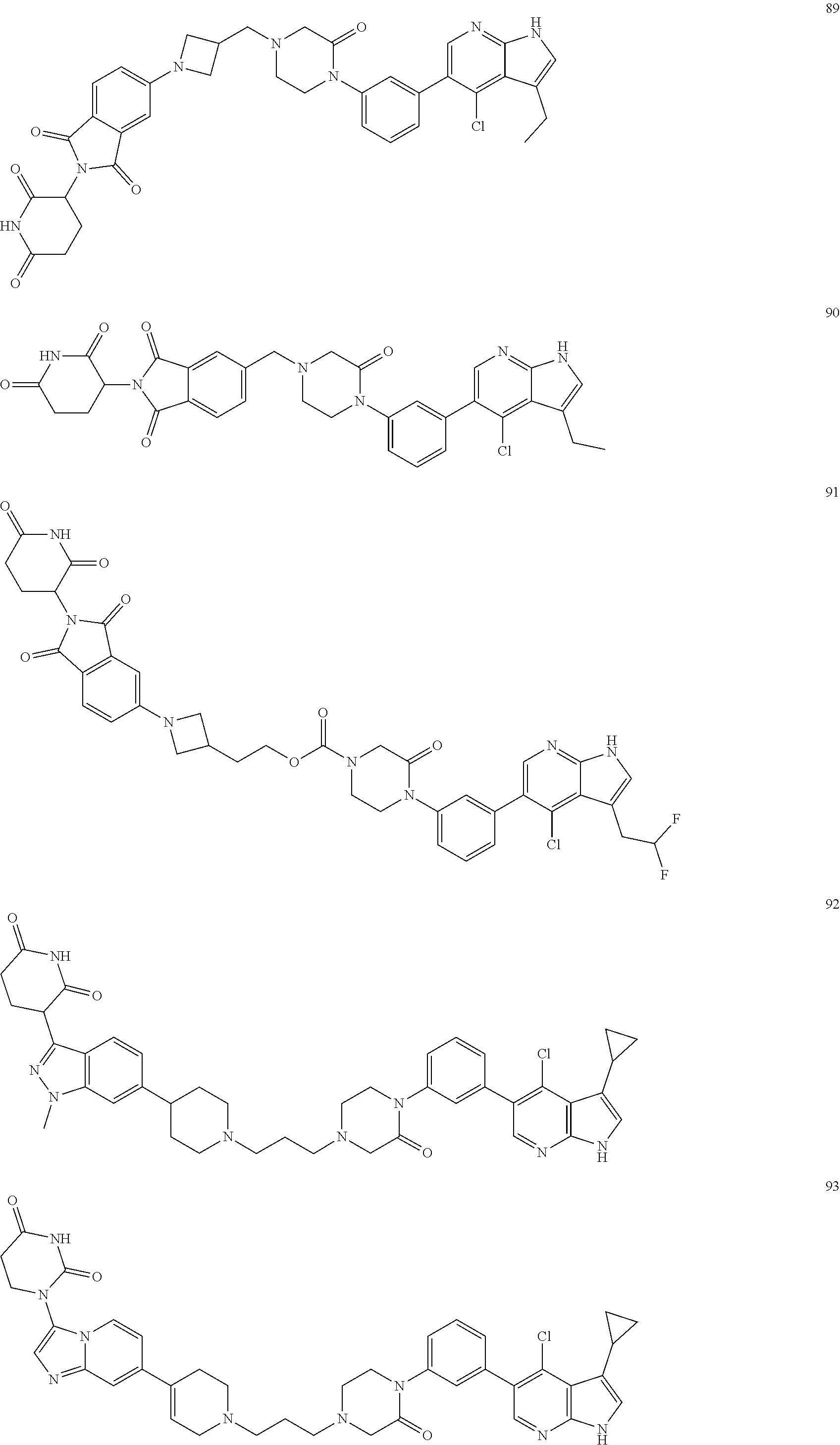 [SMILES: CCc1c[nH]c2ncc(-c3cccc(N4CCN(CC5CN(c6ccc7c(c6)C(=O)N(C6CCC(=O)NC6=O)C7=O)C5)CC4=O)c3)c(Cl)c12 ; CCc1c[nH]c2ncc(-c3cccc(N4CCN(Cc5ccc6c(c5)C(=O)N(C5CCC(=O)NC5=O)C6=O)CC4=O)c3)c(Cl)c12 ; O=C1CCC(N2C(=O)c3ccc(N4CC(CCOC(=O)N5CCN(c6cccc(-c7cnc8[nH]cc(CC(F)F)c8c7Cl)c6)C(=O)C5)C4)cc3C2=O)C(=O)N1 ; Cn1nc(C2CCC(=O)NC2=O)c2ccc(C3CCN(CCCN4CCN(c5cccc(-c6cnc7[nH]cc(C8CC8)c7c6Cl)c5)C(=O)C4)CC3)cc21 ; O=C1CCN(c2cnc3cc(C4=CCN(CCCN5CCN(c6cccc(-c7cnc8[nH]cc(C9CC9)c8c7Cl)c6)C(=O)C5)CC4)ccn23)C(=O)N1] 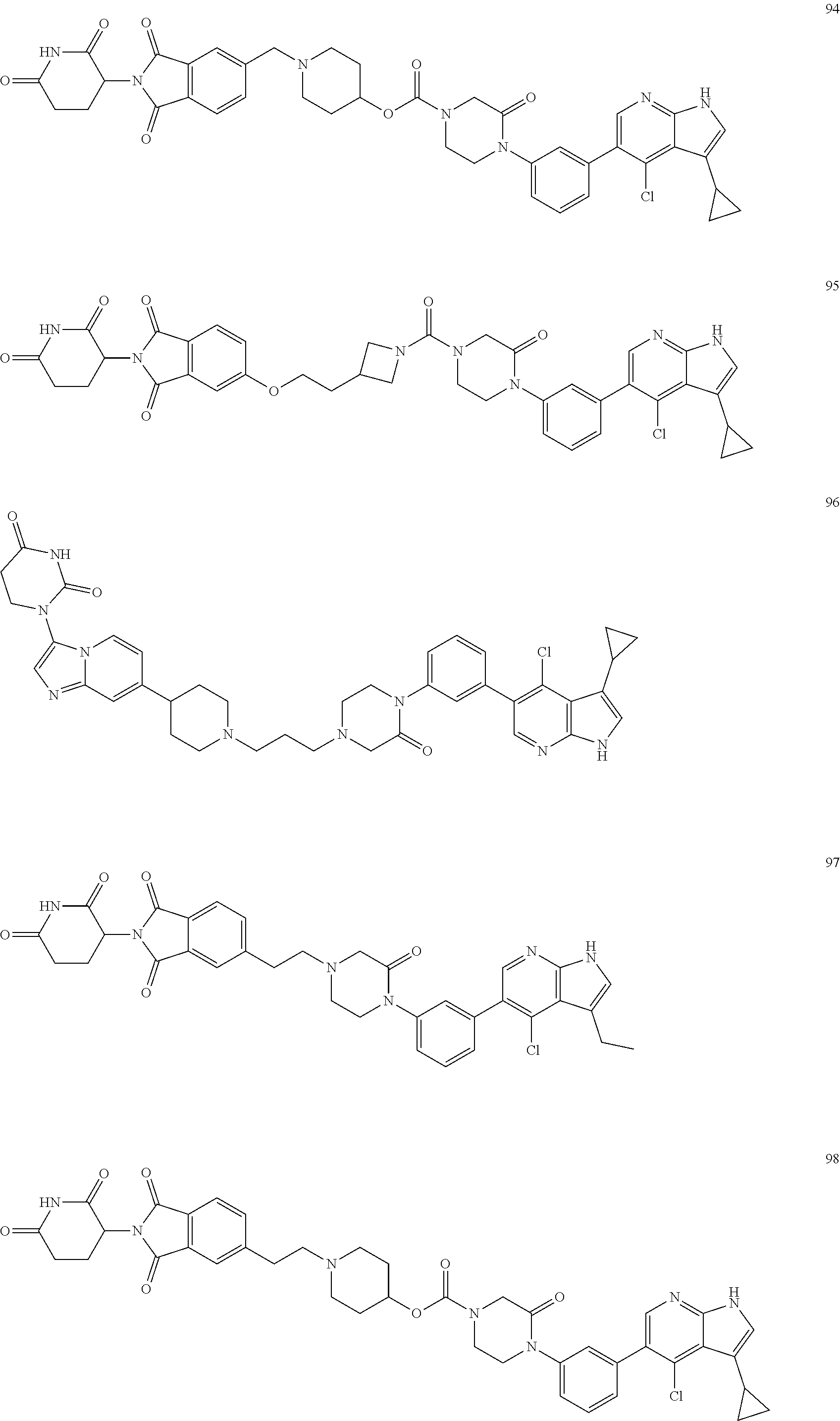 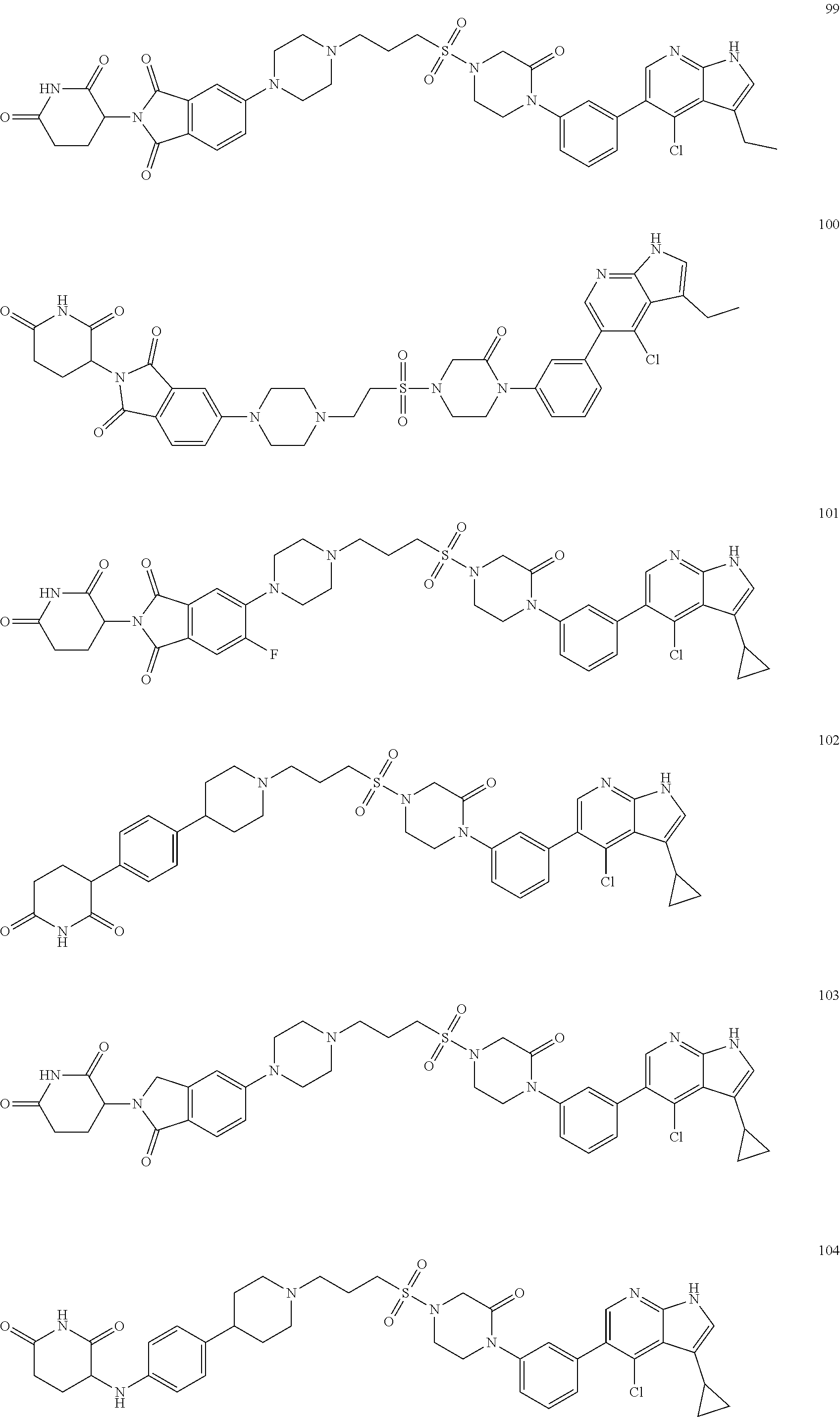 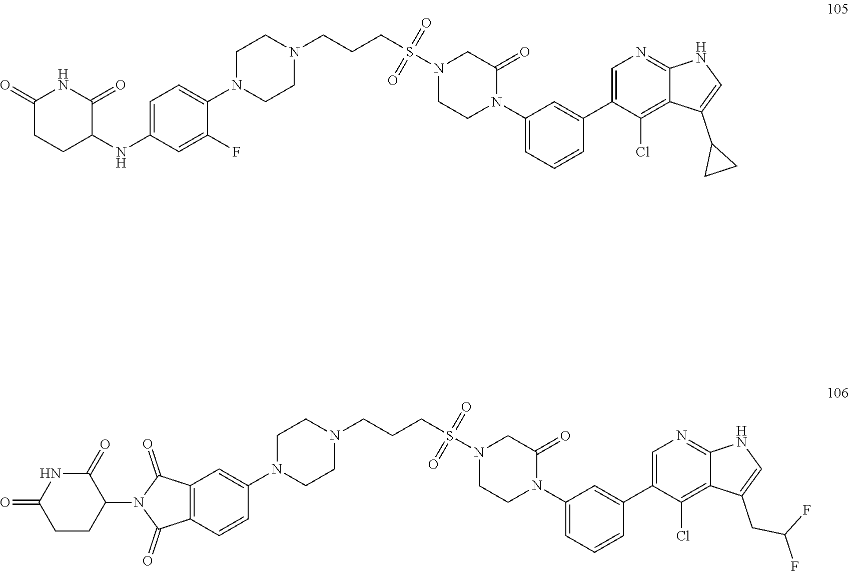 [SMILES: O=C1CCC(Nc2ccc(N3CCN(CCCS(=O)(=O)N4CCN(c5cccc(-c6cnc7[nH]cc(C8CC8)c7c6Cl)c5)C(=O)C4)CC3)c(F)c2)C(=O)N1 ; O=C1CCC(N2C(=O)c3ccc(N4CCN(CCCS(=O)(=O)N5CCN(c6cccc(-c7cnc8[nH]cc(CC(F)F)c8c7Cl)c6)C(=O)C5)CC4)cc3C2=O)C(=O)N1]
a tautomer thereof, a deuterated derivative of the compound or the tautomer, or a pharmaceutically acceptable salt of the foregoing.

6. A pharmaceutical composition comprising a compound, tautomer, deuterated derivative, or pharmaceutically acceptable salt according to any one of claims 1-5 and at least one pharmaceutically acceptable carrier.

7. A method for treating or alleviating a disease, a disorder or a condition mediated by the degradation of hematopoietic progenitor kinase 1 (HPK1), comprising administering to a subject in need thereof a therapeutically effective amount of a compound, tautomer, deuterated derivative, or pharmaceutically acceptable salt according to any one of the claims 1-5 or the pharmaceutical composition according to claim 6.

8. A method for decreasing HPK1 activity in a disease, a disorder or a condition, comprising administering to a subject in need thereof a therapeutically effective amount of a compound, tautomer, deuterated derivative, or pharmaceutically acceptable salt according to any one of the claims 1-5 or the pharmaceutical composition according to claim 6.

9. The method of claim 8, wherein the disease, the disorder, or the condition is chosen from an HPK1-related disease.

10. The method of claim 9, wherein the HPK1-related disease is chosen from cancer, a dysregulated immune response, or a disease involved in aberrant HPK1 expression, activity, and/or signaling.

11. The method of claim 10, wherein the cancer is chosen from brain cancer, breast cancer, respiratory tract and/or lung cancer, a reproductive organ cancer, bone cancer, digestive tract cancer, urinary tract cancer, eye cancer, liver cancer, kidney cancer, skin cancer, head and neck cancer, anal cancer, nervous system cancer, thyroid cancer, parathyroid cancer, a lymphoma, a sarcoma, and a leukemia.
